(19) United States
(12) Patent Application Publication
(10) Pub. No.: US 2025/0282785 A1
(43) Pub. Date: Sep. 11, 2025

(54) PYRROLIDINONE DERIVATIVES AS INHIBITORS OF NF KAPPA B INDUCING KINASE

(71) Applicant: Janssen Pharmaceutica NV, Beerse (BE)

(72) Inventors: Marc Adler, Orinda, CA (US); Wenying Chai, San Diego, CA (US); Wendy Eccles, San Diego, CA (US); Kevin Greenman, Thousand Oaks, CA (US); Michael D. Hack, San Diego, CA (US); William M. Jones, Jenkintown, PA (US); Paul J. Krawczuk, Newtown, PA (US); Alec D. Lebsack, Ladera Ranch, CA (US); Ronald L. Wolin, San Diego, CA (US); Stephen K. Murphy, San Diego, CA (US); Alexander R. Rovira, San Diego, CA (US)

(73) Assignee: Janssen Pharmaceutica NV, Beerse (BE)

(21) Appl. No.: 18/863,744

(22) PCT Filed: May 10, 2023
(86) PCT No.: PCT/EP2023/062499

§ 371 Date: Nov. 7, 2024

Related U.S. Application Data
(60) Provisional application No. 63/364,552, filed on May 11, 2022.

(51) Int. Cl. C07D 487/04; A61K 31/4155; A61K 31/4184; A61K 31/427; A61K 31/437; A61K 31/443; A61K 31/4439; A61K 31/444; A61K 31/4725; A61K 31/497; A61K 31/498; A61K 31/4985; A61K 31/5025; A61K 31/506; A61K 31/517; A61K 31/519; A61K 31/53; C07B 59/00; C07D 401/14; C07D 403/14; C07D 413/04; C07D 413/14; C07D 417/14; C07D 471/04; C07D 491/048; C07D 491/08; C07D 495/04; C07D 513/04

(52) U.S. Cl. CPC C07D 487/04; A61K 31/4155; A61K 31/4184; A61K 31/427; A61K 31/437; A61K 31/443; A61K 31/4439; A61K 31/444; A61K 31/4725; A61K 31/497; A61K 31/498; A61K 31/4985; A61K 31/5025; A61K 31/506; A61K 31/517; A61K 31/519; A61K 31/53; C07B 59/002; C07D 401/14; C07D 403/14; C07D 413/04; C07D 413/14; C07D 417/14; C07D 471/04; C07D 491/048; C07D 491/08; C07D 495/04; C07D 513/04

(57) ABSTRACT

The present disclosure relates to compounds of Formula (I) that inhibit NIK and pharmaceutical compositions comprising such compounds and methods of using the same. These compounds and pharmaceutical compositions are useful for preventing or treating diseases such as inflammatory disorders and autoimmune disorders.

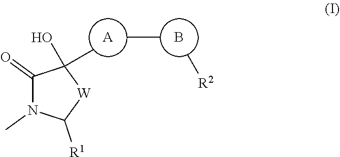

### CROSS-REFERENCE TO RELATED APPLICATIONS

This application claims the benefit of U.S. Provisional Application No. 63/364,552, filed on May 11, 2022, which is hereby incorporated herein by reference in its entirety.

### FIELD OF THE INVENTION

The present disclosure relates to the field of chemistry and medicine. More particularly, the present disclosure relates to NF-κB-inducing kinase inhibitors and their use in medical treatment.

### BACKGROUND

NF-κB inducing kinase (NIK) is a serine/threonine kinase transcription factor regulating the expression of various genes involved in immune response disorders. Because of this immune system regulatory role, inhibition of NIK blocks several downstream pathways that produce inflammatory molecules. Clinical validation with biologics has confirmed a key role for several NIK-dependent pathways in autoimmune diseases. See, e.g., S. V. Navarra, et al., The Lancet, 2011; 377(9767):721-31. One way to mitigate or eliminate the adverse effects associated with NIK activity is to increase NIK inhibition.

Thus, there is a need to develop effective NIK inhibitors that can be used to treat various diseases.

### SUMMARY

The present application discloses a compound of Formula I:

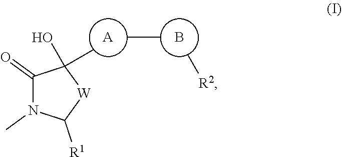

or a pharmaceutically acceptable salt thereof, wherein A, B, W, R¹, and R² are as defined herein.

The present application also discloses a pharmaceutical composition comprising a compound of Formula I, or a pharmaceutically acceptable salt thereof, and a pharmaceutically acceptable carrier.

The present application also discloses a method for treating a disease, disorder, or medical condition mediated by NIK activity, comprising administering to a subject in need of such treatment an effective amount of (i) a compound of Formula I, or a pharmaceutically acceptable carrier thereof, or (ii) a pharmaceutical composition comprising a compound of Formula I, or a pharmaceutically acceptable salt thereof, and a pharmaceutically acceptable carrier. In some embodiments, the disease, disorder, or medical condition mediated by NIK activity is selected from the group consisting of inflammatory disorders, autoimmune disorders, cancers, metabolic disorders, and osteoporosis. In some embodiments, the disease, disorder, or medical condition mediated by NIK activity is selected from the group consisting of systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.

Additional embodiments, features, and advantages of the present disclosure will be apparent from the following detailed description and through practice of the present disclosure.

### DETAILED DESCRIPTION

Discussion of documents, acts, materials, devices, articles or the like which has been included in the present specification is for the purpose of providing context for the present disclosure. Such discussion is not an admission that any or all of these matters form part of the prior art with respect to any inventions disclosed or claimed.

NF-κB-inducing kinase (referred to as NIK, also known as MAP3K14) is a regulator and driver of the non-canonical NIK cascade, and thus represents an attractive target for therapeutic intervention. Embodiments described herein relate to compounds that inhibit NIK and pharmaceutical compositions comprising such compounds. Compounds described herein and pharmaceutical compositions thereof are useful for preventing or treating diseases such as inflammatory disorders and autoimmune disorders.

NIK-dependent transcriptional activation is a tightly controlled signaling pathway, through sequential events including phosphorylation and protein degradation. In a NIK activation pathway, known as a non-canonical pathway, activation is accomplished by phosphorylating the catalytic complex subunit IKKα, leading to the partial proteolysis of the gene product p100, liberating DNA-binding protein p52 which then heterodimerizes with another DNA-binding protein RelB, translocates to the nucleus and mediates gene expression. The non-canonical pathway is activated by ligands such as CD40 ligands, B-cell activating factor (BAFF), lymphotoxin β receptor ligands, TNF-related weak inducer of apoptosis (TWEAK) cytokine, and receptor activator of nuclear factor kappa-B ligand (RANKL), also known as tumor necrosis factor ligand superfamily member 11 (TNFSF11). NIK has been shown to be required for activation of the pathway by these ligands (S.-C. Sun, Nat Rev Immunol. 2017, 17(9), 545-558). Because of its role, NIK expression is tightly regulated. Under normal non-stimulated conditions NIK protein levels are very low. This is due to its interaction with baculoviral-IAP-repeat-containing-3 (BIRC3, also known as CIAP2) and a range of TNF receptor associated factors (TRAF2 and TRAF3), which are ubiquitin ligases and result in degradation of NIK. It is believed that when the non-canonical pathway is stimulated by ligands under pathological/abnormal conditions, the activated receptors now compete for TRAFs, dissociating the TRAF-BIRC3-NIK complexes and thereby increasing the levels of NIK (For a more detailed analysis of this background, see e.g., S.-C. Sun (cited above) and Thu and Richmond, Cytokine Growth F. R. 2010, 21, 213-226). NIK plays a role propitiating immune response disorders, so a NIK level increase is undesirable, and one way to mitigate or eliminate the adverse effect associated with such increase is NIK inhibition.

BAFF/BAFF-R is a clinically validated therapeutic target whose inhibition is deemed beneficial for systemic lupus erythematosus (SLE) treatment. Belimumab (anti-BAFF antibody) has been approved to treat serum positive SLE patients (S. V. Navarra, et al., The Lancet, 2011; 377(9767):721-31). The CD40L/CD40 pathway plays a key role in T-dependent B cell activation, dendritic cell maturation and tissue inflammation/immunity (R. Elgueta, et al., Immunol. Rev. 2009; 229(1):152-72). An anti-CD40L antibody has demonstrated promising efficacy in phase 2 clinical studies in SLE patients (P. I. Sidiropoulos and D. T. Boumpas, Lupus 2004 May; 13(5):391-7). Mice lacking NIK (R. Shinkura, et al., Nature Genetics 1999; 22(1):74-7; H. D. Brightbill, et al., J Immunol. 2015; 195(3):953-64) or conditional knockout of NIK (H. D. Brightbill, et al., J Immunol. 2015; 195(3):953-64) or human patients carrying NIK gene mutations (K. L. Willmann, et al., Nature Comm. 2014; 5:5360) showed deficiency in NIK non-canonical activation pathways such as BAFF and CD40L pathway, reduced B lymphocytes in peripheral blood, and lymphoid organs and lower T cell dependent antibody responses supporting NIK as a therapeutic target for SLE.

NIK has been characterized as being “important in the immune and bone-destructive components of inflammatory arthritis and represents a possible therapeutic target for these diseases.” K. Aya, et al. (J. Clin. Invest. 2005, 115, 1848-1854). Mice lacking functional NIK have no peripheral lymph nodes, defective B and T cells, and impaired receptor activator of NIK ligand-stimulated osteoclastogenesis. K. Aya, et al. (J. Clin. Invest. 2005, 115, 1848-1854) investigated the role of NIK in murine models of inflammatory arthritis using NIK−/− mice. The serum transfer arthritis model was initiated by preformed antibodies and required only intact neutrophil and complement systems in recipients. While NIK−/− mice had inflammation equivalent to that of NIK+/+ controls, Ada, et al., (cited above) showed significantly less periarticular osteoclastogenesis and less bone erosion. In contrast, NIK−/− mice were completely resistant to antigen-induced arthritis (AIA), which requires intact antigen presentation and lymphocyte function but not lymph nodes. Additionally, transfer of NIK+/+ splenocytes or T cells to Rag2−/− mice conferred susceptibility to AIA, while transfer of NIK−/− cells did not. NIK−/− mice were also resistant to a genetic, spontaneous form of arthritis, generated in mice expressing both the KRN T cell receptor and H-2g7. Transgenic mice were used with OC-lineage expression of NIK lacking its TRAF3 binding domain (NT3), to demonstrate that constitutive activation of NIK drives enhanced osteoclastogenesis and bone resorption, both in basal conditions and in response to inflammatory stimuli. See Aya, et al., cited above. Furthermore, constitutive activation of NIK drives enhanced osteoclastogenesis and bone resorption, both in basal conditions and in response to inflammatory stimuli. (C. Yang, et al., PLoS ONE 2010, 5(11): e15383, doi:10.1371/journal.pone.0015383).

NIK is also a therapeutic target for other BAFF, CD40L or lymphotoxin β receptor ligands driven autoimmune disorders such as Sjogren's syndrome (J. Groom, et al., J. Clin. Invest. 2002; 109(1):59-68) and proliferative lupus glomerulonephritis (D. T. Boumpas, et al., Arthritis & Rheumatism 2003; 48(3):719-27).

### Definitions

Unless defined otherwise, all technical and scientific terms used herein have the same meaning as commonly understood to one of ordinary skill in the art to which the present disclosure pertains. Otherwise, certain terms used herein have the meanings as set forth in the specification.

In an attempt to help the reader of the application, the description has been separated in various paragraphs or sections or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

It must be noted that as used herein and in the appended claims, the singular forms “an,” and “the” include plural reference unless the context clearly dictates otherwise.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The term “administering” with respect to the methods of the present disclosure, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a compound of the disclosure, or pharmaceutically acceptable salt thereof, composition thereof, or medicament thereof, at different times during the course of a therapy or concurrently or sequentially as a combination therapy.

The term “subject” refers to a patient, which may be an animal, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

The term “therapeutically effective amount” or “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has a disorder or disease as described herein, a symptom thereof; or the potential to develop such disorder or disease, where the purpose of the application or administration is to cure, heal, alleviate, relieve, alter, remedy, ameliorate, improve or affect the disorder or disease, its symptoms, or the potential to develop said disorder or disease. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “C_{(a-b)}” (where a and b are integers referring to a designated number of carbon atoms) refers, for example, to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “alkyl” is a straight or branched saturated hydrocarbon. For example, an alkyl group can have 1 to 12 carbon atoms (i.e., (C₁-C₁₂)alkyl), 1 to 6 carbon atoms (i.e., (C₁-C₆)alkyl), 1 to 4 carbon atoms (i.e., (C₁-C₄)alkyl), or 1 to 3 carbon atoms (i.e., (C₁-C₃)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₍₁₋₆₎alkyl. In another embodiment, alkyl refers to C₍₁₋₄₎alkyl. In another embodiment, alkyl refers to C₍₁₋₃₎alkyl.

The term “halo” or “halogen” refers to bromo (—Br), chloro (—Cl), fluoro (—F), or iodo (-d). In an embodiment, halo refers to fluoro.

The term “haloalkyl” refers to a straight- or branched-chain alkyl group having from 1 to 12 carbon atoms, 1 to 6 carbon atoms, 1 to 4 carbon atoms, or 1 to 3 carbon atoms in the chain optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples. In an embodiment, haloalkyl refers to C₍₁₋₆₎haloalkyl. In another embodiment, haloalkyl refers to C₍₁₋₄₎haloalkyl. In another embodiment, alkyl refers to C₍₁₋₃₎haloalkyl.

The term “cycloalkyl” refers to a saturated or partially unsaturated all carbon ring system having, for example, 3 to 10 carbon atoms (i.e., C₍₃₋₁₀₎cycloalkyl), 3 to 8 carbon atoms (i.e., C₍₃₋₈₎cycloalkyl), or 3 to 6 carbon atoms (i.e., C₍₃₋₆₎cycloalkyl), wherein the cycloalkyl ring system has a single ring or multiple rings in a fused, spirocyclic, or bridged configuration. Exemplary cycloalkyls include, but are not limited to, cyclopropyl, cyclobutyl, cyclopentyl, cyclohexyl, cycloheptyl, and cyclooctyl. Some cycloalkyl groups may exist as spirocycloalkyls, wherein two cycloalkyl rings are fused through a single carbon atom; for example and without limitation, an example of a spiropentyl group is

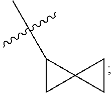 [SMILES: *C1CC12CC2]

for example and without limitation, examples of spirohexyl groups include

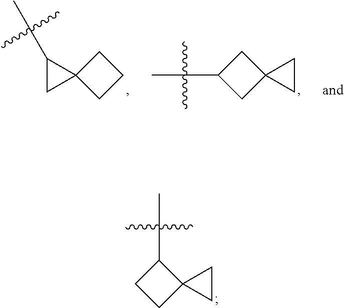 [SMILES: *C1CC12CCC2 ; *C1CC2(CC2)C1 ; *C1CCC12CC2]

for example and without limitation examples of cycloheptyl groups include

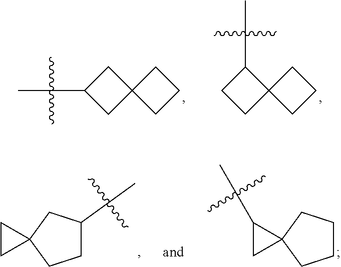

for example and without limitation examples of cyclooctyl groups include

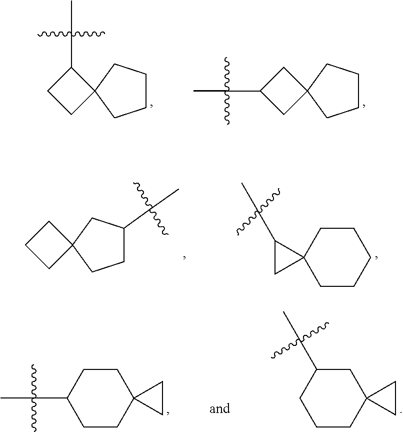 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

In an embodiment, cycloalkyl refers to C₍₃₋₁₀₎cycloalkyl. In another embodiment, cycloalkyl refers to C₍₃₋₈₎cycloalkyl. In another embodiment, cycloalkyl refers to C₍₃₋₆₎cycloalkyl.

The term “aryl,” unless otherwise stated, refers to a polyunsaturated, typically aromatic, hydrocarbon group which can be a single ring or multiple rings (up to three rings) which are fused together or linked covalently. The term aromatic is well known to a person skilled in the art and designates cyclically conjugated systems of 4n+2 electrons, that is with 6, 10, 14 etc. n-electrons (rule of Hückel). Examples of aryl groups include phenyl, naphthyl, anthracenyl. In an embodiment, aryl refers to C₍₆₋₁₀₎aryl. In another embodiment, aryl refers to phenyl.

The term “heterocyclyl” or “heterocycloalkyl” refers to a single saturated or partially unsaturated ring having 3 to 12 ring members, 3 to 10 ring members, 3 to 8 ring members, or 3 to 6 ring members and which contains carbon atoms and at least one atom other than carbon in the ring, wherein the atom is selected from the group consisting of N, O, and S. The terms “heterocyclyl” and “heterocycloalkyl” include cyclic esters (e.g., lactones) and cyclic amides (e.g., lactams). Exemplary heterocycles include, but are not limited to oxetanyl, aziridinyl, azetidinyl, pyrrolidinyl, pyrrolidonyl, piperidinyl, piperazinyl, morpholinyl, tetrahydropyranyl, tetrahydrofuranyl, and thiomorpholinyl. Unless otherwise noted, the heterocyclyl group is attached to its pendant group at any heteroatom or carbon atom that results in a stable structure. In an embodiment, heterocyclyl refers to 3- to 10-membered heterocyclyl. In another embodiment, heterocyclyl refers to 3- to 8-membered heterocyclyl. In another embodiment, heterocyclyl refers to 3- to 6-membered heterocyclyl.

As used herein, the term “5- to 12-membered bi- or tricyclic ring system containing one or more heteroatoms” refers to a saturated or partially saturated bridged polycyclic, fused polycyclic, or spiro polycyclic ring system having 5 to 12 ring members (or 7 to 12 ring members or 7 to 10 ring members) and which contains carbon atoms and from 1 to 7 heteroatoms, 1 to 5 heteroatoms, 1 to 4 heteroatoms, or 1 to 3 heteroatoms, wherein the heteroatoms are independently selected from the group consisting of N, O, and S. The ring system may include a fully unsaturated aromatic ring; however, at least one other ring in the polycyclic ring system must be saturated or partially saturated. In some embodiments, the term refers to a fused bicyclic ring system. In some embodiments, the term refers to a fused bicyclic ring system wherein one of the rings is an aromatic ring. The term includes cyclic esters (e.g., lactones) and cyclic amides (e.g., lactams). Nonlimiting examples of 5- to 12-membered bi- or tricyclic ring system containing one or more heteroatoms include 3-oxabicyclo[3.1.0]hexyl, indolinyl, 6,7-dihydro-5H-cyclopenta[b]pyridinyl, 4,5,6,7-tetrahydro-1H-indazolyl, 6,7-dihydro-5H-cyclopenta[c]pyridazinyl, 4,5,6,7-tetrahydropyrazolo[1,5-a]pyridinyl, 4,5,6,7-tetrahydropyrazolo[1,5-a]pyrimidinyl, 3,4-dihydro-2H-pyrano[3,2-b]pyridinyl, 3-methyl-6,7-dihydro-5H-pyrazolo[5,1-b][1,3]oxazinyl, and 6,7-dihydropyrazolo[1,5-a]pyrazin-4(5H)-onyl. Unless otherwise noted, the bi- or tricyclic ring system is attached to its pendant group at any heteroatom or carbon atom that results in a stable structure. In an embodiment, a 5- to 12-membered bi- or tricyclic ring system containing one or more heteroatoms refers to a 5- to 12-membered bicyclic ring system containing one or more heteroatoms. In another embodiment, a 5- to 12-membered bi- or tricyclic ring system containing one or more heteroatoms refers to a 7- to 12-membered bicyclic ring system containing one or more heteroatoms. In another embodiment, a 5- to 12-membered bi- or tricyclic ring system containing one or more heteroatoms refers to a 7- to 10-membered bicyclic ring system containing one or more heteroatoms.

The term “heteroaryl” refers to a monocyclic or bicyclic aryl ring system having 5 to 12 ring members, 5 to 10 ring members, or 5 to 6 ring members, and which contains carbon atoms and from 1 to 5 heteroatoms, 1 to 3 heteroatoms, or 1 to 2 heteroatoms, wherein the heteroatoms are independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In some embodiments, in the case of 5-membered rings, the heteroaryl ring contains one member of nitrogen, oxygen or sulfur and, in addition, up to 3 additional nitrogens. In some embodiments, in the case of 6-membered rings, the heteroaryl ring contains from 1 to 3 nitrogen atoms. For the case wherein the 6-membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl, quinazolinyl, pyrazolopyridinyl, and pyrazolopyrimidinyl. Those skilled in the art will recognize that the species of heteroaryl groups listed are not exhaustive, and that additional species within the scope of these defined terms may also be selected. Unless otherwise noted, the heteroaryl is attached to its pendant group at any heteroatom or carbon atom that results in a stable structure. In an embodiment, heteroaryl refers to 5- to 10-membered heteroaryl. In another embodiment, heteroaryl refers to 5- to 8-membered heteroaryl. In another embodiment, heteroaryl refers to 5- to 6-membered heteroaryl. In another embodiment, heteroaryl refers to 5-membered heteroaryl.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Where the compounds disclosed herein have at least one stereocenter, they may accordingly exist as enantiomers or diastereomers. It is to be understood that all such isomers and mixtures thereof are encompassed within the scope of the present disclosure.

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

Where the processes for the preparation of the compounds according to the disclosure give rise to mixture of stereoisomers, these isomers may be separated by conventional techniques such as preparative chromatography. The compounds may be prepared in racemic form, a scalemic mixture, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral column vial HPLC or SFC. In some instances rotamers of compounds may exist which are observable by H NMR leading to complex multiplets and peak integration in the ¹H NMR spectrum.

The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain pairs of enantiomers and diastereomers are presented together in the Examples. These enantiomers/diastereomers may be designated in the following synthetic method and characterized as enantiomer 1 or enantiomer 2 (or, alternately, diastereomer 1 or diastereomer 2). The presentation of stereoisomers in this manner conveys the separate preparation or isolation of the compounds as pure single enantiomers or diastereomers at the identified stereocenter(s). However, unless otherwise specified, when a pure single enantiomer (or diastereomer) is presented together with the corresponding pure single enantiomer (or diastereomer) in the Examples of the present disclosure, the order in which the chemical structures/IUPAC names are presented do not necessarily correspond to the order in which the Example numbers are listed. By way of example, where R and S enantiomers of a compound are presented side-by-side under the header “Example X and Example Y,” then Example X may be either the R enantiomer or the S enantiomer, and Example Y is the opposite enantiomer, regardless of the order in which the IUPAC names or chemical structures of the compounds are presented, unless otherwise specified in the method and characterization that follows.

During any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Furthermore, it is intended that within the scope of the present disclosure, any element, in particular when mentioned in relation to a compound of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen or “H” includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —C¹H₃, but also C¹HD₂, C¹H₂D, CD₃ and other isotopic forms; not only C¹H₂C¹H₃, but also C¹HDC¹HD₂, C¹HDC¹H₂D, C¹H₂C¹H₃, CD₂CD₃, etc. Likewise, where nonexplicit hydrogen atoms are present in a chemical structure, those hydrogen atoms may be ¹H, ²H (i.e., deuterium or D), or ³H (i.e., tritium or T). By way of illustration, the group

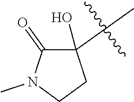 [SMILES: *C1(O)CCN(C)C1=O]

also encompasses

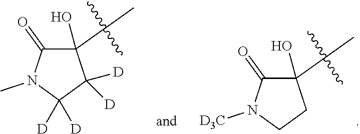 [SMILES: *C1(O)C(=O)N(C)C([2H])([2H])C1([2H])[2H] ; *C1(O)CCN(C([2H])([2H])[2H])C1=O]

Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C ¹⁸F, ³⁵S, ¹²²I, ¹²³I, ¹²⁵I, ¹³¹I, ⁷⁵Br, ⁷⁶Br, ⁷⁷Br and ⁸²Br. In some embodiments, the radioactive isotope is selected from the group of ³H, ¹¹C and ¹⁸F.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

The term “pharmaceutically acceptable” means approved or approvable by a regulatory agency of Federal or a state government or the corresponding agency in countries other than the United States, or that is listed in the U. S. Pharmcopoeia or other generally recognized pharmacopoeia for use in animals, and more particularly, in humans.

A “pharmaceutically acceptable salt” is intended to mean a salt of a free acid or base of a compound disclosed herein that is non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of the disclosure may possess a sufficiently acidic group, a sufficiently basic group, or both types of functional groups, and accordingly react with a number of inorganic or organic bases, and inorganic and organic acids, to form a pharmaceutically acceptable salt.

### Compounds of the Disclosure

The present application discloses a compound of Formula I:

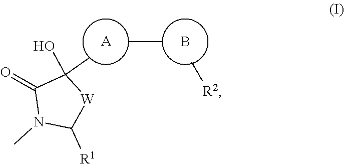

or a pharmaceutically acceptable salt thereof, wherein:
A is a 5-membered heteroaryl that is optionally substituted with one to three groups selected from —C₍₁₋₄₎alkyl, and —C₍₁₋₄₎haloalkyl;
B is furanyl, piperidinyl, or a group having the following structure:

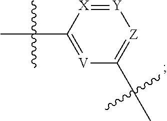

W is CH₂, CHF, or CF₂;
V is N, C—H, or C—R^{V};
X is N, C—H, or C—R^{X};
Y is N, C—H, or C—R^{Y};
Z is N, C—H, or C—R^{Z};
R^{V}, R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein the —N(H)(5- to 10-membered heteroaryl), the —C₍₆₋₁₀₎aryl, the 5- to 12-membered bi- or tricyclic ring system containing one or more heteroatoms, and the 5- to 10-membered heteroaryl are each optionally substituted with one to five R³ groups;
each R³ is independently for each occurrence halo, —OH, —N(R^{N1})(R^{N2}), —CN, —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2}, R^{N3}, and R^{N4} are each independently for each occurrence hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R³ groups.

In some embodiments, disclosed herein is a compound of Formula I, or a pharmaceutically acceptable salt thereof, wherein:
A is a 5-membered heteroaryl that is optionally substituted with one to three groups selected from —C₍₁₋₄₎alkyl, and —C₍₁₋₄₎haloalkyl;
B is furanyl, piperidinyl, or a group having the following structure:

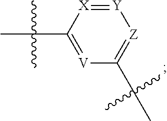

W is CH₂ or CF₂;
V is N, C—H, or C—R^{V};
X is N, C—H, or C—R^{X};
Y is N, C—H, or C—R^{Y};
Z is N, C—H, or C—R^{Z};
R^{V}, R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein the —N(H)(5- to 10-membered heteroaryl), the —C₍₆₋₁₀₎aryl, the 5- to 12-membered bi- or tricyclic ring system containing one or more heteroatoms, and the 5- to 10-membered heteroaryl are each optionally substituted with one to five R³ groups;
each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl-N(R^{N1})(R^{N3}), —C(O)OH, —C(O)N(R^{N1})(R^{N4}), C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R³ groups.

In some embodiments, disclosed herein is a compound of Formula I, or a pharmaceutically acceptable salt thereof, wherein:
A is a 5-membered heteroaryl that is optionally substituted with one to three —C₍₁₋₄₎alkyl groups;
B is furanyl, piperidinyl, or a group having the following structure:

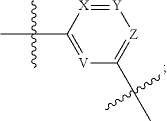

W is CH₂ or CF₂;
V is N, C—H, or C—R^{V};
X is N, C—H, or C—R^{X};
Y is N, C—H, or C—R^{Y};
Z is N, C—H, or C—R^{Z};
R^{V} is halo;
R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein the —N(H)(5- to 10-membered heteroaryl), the —C₍₆₋₁₀₎aryl, the 5- to 12-membered bi- or tricyclic ring system containing one or more heteroatoms, and the 5- to 10-membered heteroaryl are each optionally substituted with one to five R³ groups;
each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R³ groups.

In some embodiments, disclosed herein is a compound of Formula I, or a pharmaceutically acceptable salt thereof, wherein:
A is a 5-membered heteroaryl that is optionally substituted with one —C₍₁₋₄₎alkyl groups;
B is

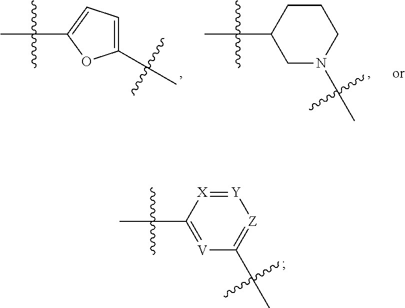

W is CH₂ or CF₂;
V is N, C—H, or C—F;
X is N, C—H, or C—R^{X};
Y is N, C—H, or C—R^{Y};
Z is N, C—H, or C—R^{Z};
R^{X}, R^{Y}, and R^{Z} are each, independently, fluorine, —CH₃, or —OCH₃;
R¹ is hydrogen or —CF₃;
R² is a 5- to 12-membered bi- or tricyclic ring system containing one or more heteroatoms or 5- to 10-membered heteroaryl, wherein the 5- to 12-membered bi- or tricyclic ring system containing one or more heteroatoms and the 5- to 10-membered heteroaryl are each optionally substituted with one to five R³ groups;
each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R³ groups.

In some embodiments, disclosed herein is a compound of Formula I, or a pharmaceutically acceptable salt thereof, wherein:
A is a 5-membered heteroaryl that is optionally substituted with one to three groups selected from —C₍₁₋₄₎alkyl and —C₍₁₋₄₎haloalkyl;
B is furanyl, piperidinyl, or a group having the following structure:

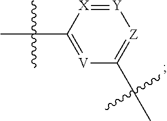

W is CH₂, CHF, or CF₂;
V is N, C—H, or C—R^{V};
X is N, C—H, or C—R^{X};
Y is N, C—H, or C—R^{Y};
Z is N, C—H, or C—R^{Z};
R^{V}, R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one to five R^{3a} groups, the —C₍₆₋₁₀₎aryl is optionally substituted with one to five R^{3b} groups, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups;
R^{3a}, R^{3b}, R^{3c}, and R^{3d} are each independently for each occurrence halo, —OH, —N(R^{N1})(R^{N2}), —CN, —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀)cycycloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2}, R^{N3}, and R^{N4} are each independently for each occurrence hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

In some embodiments, disclosed herein is a compound of Formula I, or a pharmaceutically acceptable salt thereof, wherein:
A is a 5-membered heteroaryl that is optionally substituted with one to three groups selected from —C₍₁₋₄₎alkyl and —C₍₁₋₄₎haloalkyl;
B is furanyl, piperidinyl, or a group having the following structure:

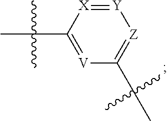

W is CH₂ or CF₂;
V is N, C—H, or C—R^{V};
X is N, C—H, or C—R^{X};
Y is N, C—H, or C—R^{Y};
Z is N, C—H, or C—R^{Z};
R^{V}, R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one to five R^{3a} groups, the —C₍₆₋₁₀₎aryl is optionally substituted with one to five R^{3b} groups, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups; R^{3a} and R^{3b} are each independently for each occurrence halo, —N(R^{N1})(R^{N2}), C₍₁₋₆₎alkyl, —C₍₁₋₆₎haloalkyl, —OC₍₁₋₆₎alkyl, —OC₍₁₋₆₎haloalkyl, —C₍₁₋₃₎alkyl-N(R^{N1})(R^{N3}), or —C(O)N(R^{N1})(R^{N4}),
R^{3c} and R^{3d} are each independently for each occurrence halo, —OH, —N(R^{N1})(R^{N2}), —CN, —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎ alkyl, —OC₍₁₋₆₎haloalkyl, —OC₍₃₋₁₀₎cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2}, R^{N3}, and R^{N4} are each independently for each occurrence hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

In some embodiments, disclosed herein is a compound of Formula I, or a pharmaceutically acceptable salt thereof, wherein:
A is a 5-membered heteroaryl that is optionally substituted with one to three groups selected from —C₍₁₋₄₎alkyl and —C₍₁₋₄₎haloalkyl;
B is furanyl, piperidinyl, or a group having the following structure:

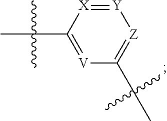

W is CH₂ or CF₂;
V is N, C—H, or C—R^{V};
X is N, C—H, or C—R^{X};
Y is N, C—H, or C—R^{Y};
Z is N, C—H, or C—R^{Z};
R^{V} is halo;
R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one to five R^{3a} groups, the —C₍₆₋₁₀₎aryl is optionally substituted with one to five R^{3b} groups, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups;
R^{3a} and R^{3b} are each independently for each occurrence —N(R^{N1})(R^{N2}) or —C(O)N(R^{N1})(R^{N4}),
each R^{3c} is independently for each occurrence halo, —N(R^{N1})(R^{N2}), —C₍₁₋₆₎alkyl, —C₍₁₋₆₎haloalkyl, —OC₍₁₋₆₎alkyl, —OC₍₁₋₆₎haloalkyl, —C₍₁₋₃₎alkyl-N(R^{N1})(R^{N3}), —C(O)OH, —C(O)N(R^{N1})(R^{N4}), or —C₍₁₋₃₎alkyl(phenyl);
each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎ alkyl-N(R^{N1})(R^{N3}), —C(O)N(R^{N1})(R^{N4}), C₍₁₋₃₎alkyl(3- to 8-membered heterocyclyl), 3- to 8-membered heterocyclyl, or 5- to 6-membered heteroaryl, wherein the —C₍₁₋₆₎alkyl, —C₍₁₋₆₎ haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl(3- to 8-membered heterocyclyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

In some embodiments, disclosed herein is a compound of Formula I, or a pharmaceutically acceptable salt thereof, wherein:
A is a 5-membered heteroaryl that is optionally substituted with one to three —C₍₁₋₄₎alkyl groups;
B is furanyl, piperidinyl, or a group having the following structure:

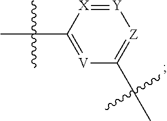

W is CH₂ or CF₂;
V is N, C—H, or C—R^{V};
X is N, C—H, or C—R^{X};
Y is N, C—H, or C—R^{Y};
Z is N, C—H, or C—R^{Z};
R^{V} is halo;
R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one —N(R^{N1})(R^{N2}) group, the —C₍₆₋₁₀₎aryl is optionally substituted with one —C(O)N(R^{N1})(R^{N4}) group, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups; each R^{3c} is independently for each occurrence —N(R^{N1})(R^{N2}), —C₍₁₋₆₎alkyl, —C₍₁₋₆₎ haloalkyl, —OC₍₁₋₆₎alkyl, —C(O)OH, —C(O)N(R^{N1})(R^{N4}), or —C₍₁₋₃₎alkyl(phenyl); each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)N(R^{N1})(R^{N4}), —C₍₁₋₃₎alkyl(3- to 8-membered heterocyclyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

In some embodiments, disclosed herein is a compound of Formula I, or a pharmaceutically acceptable salt thereof, wherein:
A is a 5-membered heteroaryl that is optionally substituted with one —C₍₁₋₄₎alkyl groups;
B is:

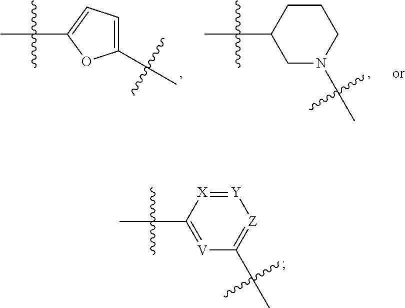

W is CH₂ or CF₂;
V is N, C—H, or C—F;
X is N, C—H, or C—R^{X};
Y is N, C—H, or C—R^{Y};
Z is N, C—H, or C—R^{Z};
R^{X}, R^{Y}, and R^{Z} are each, independently, fluorine, —CH₃, or —OCH₃;
R¹ is hydrogen or —CF₃;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one —NH₂ group, the —C₍₆₋₁₀₎aryl is optionally substituted with one —C(O)NH₂ group, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups;
each R^{3c} is independently for each occurrence —NH₂, —C₍₁₋₆₎alkyl, —C₍₁₋₆₎haloalkyl, —OC₍₁₋₆₎alkyl, —C(O)OH, —C(O)NH₂, or —C₍₁₋₃₎alkyl(phenyl);
each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)N(R^{N1})(R^{N4}), —C₍₁₋₃₎alkyl(3- to 8-membered heterocyclyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

In some embodiments, disclosed herein is a compound of Formula I, or a pharmaceutically acceptable salt thereof, which is a compound of Formula Ia-1:

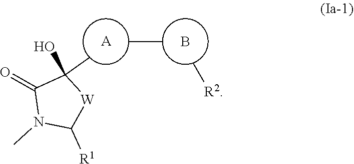

In some embodiments, disclosed herein is a compound of Formula I, or a pharmaceutically acceptable salt thereof, which is a compound of Formula Ia-2:

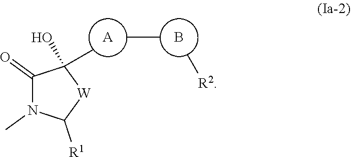

In some embodiments, disclosed herein is a compound of Formula I, or a pharmaceutically acceptable salt thereof, wherein A is a 5-membered heteroaryl, which is optionally substituted with one —C₍₁₋₄₎alkyl group. In some embodiments, disclosed herein is a compound of Formula I, or a pharmaceutically acceptable salt thereof, wherein A is a 5-membered heteroaryl, which is optionally substituted with one methyl group. In some embodiments, disclosed herein is a compound of Formula I, or a pharmaceutically acceptable salt thereof, wherein A is a 5-membered heteroaryl.

In some embodiments, disclosed herein is a compound of Formula I, or a pharmaceutically acceptable salt thereof, wherein A is oxazolyl, isoxazolyl, thiazolyl, isothiazolyl, pyrazolyl, imidazolyl, or triazolyl, each of which is optionally substituted with one —C₍₁₋₄₎alkyl group. In some embodiments, disclosed herein is a compound of Formula I, or a pharmaceutically acceptable salt thereof, wherein A is oxazolyl, isoxazolyl, thiazolyl, isothiazolyl, pyrazolyl, imidazolyl, or triazolyl, each of which is optionally substituted with one methyl group. In some embodiments, disclosed herein is a compound of Formula I, or a pharmaceutically acceptable salt thereof, wherein A is oxazolyl, isoxazolyl, thiazolyl, isothiazolyl, pyrazolyl, imidazolyl, or triazolyl. In some embodiments, disclosed herein is a compound of Formula I, or a pharmaceutically acceptable salt thereof, wherein A is isoxazolyl or pyrazolyl. In some embodiments, disclosed herein is a compound of Formula I, or a pharmaceutically acceptable salt thereof, wherein A is isoxazolyl. In some embodiments, disclosed herein is a compound of Formula I, or a pharmaceutically acceptable salt thereof, wherein A is pyrazolyl.

In some embodiments, disclosed herein is a compound of Formula I, or a pharmaceutically acceptable salt thereof, wherein A is

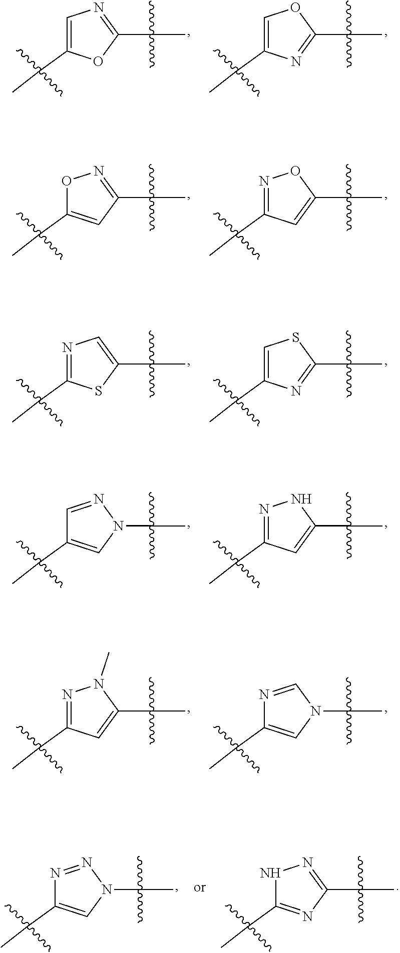

In some embodiments, disclosed herein is a compound of Formula I, or a pharmaceutically acceptable salt thereof, wherein A is

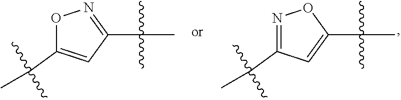 [SMILES: *c1cc(*)on1 ; *c1cc(*)on1]

In some embodiments, disclosed herein is a compound of Formula I, or a pharmaceutically acceptable salt thereof, wherein A is

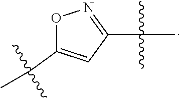 [SMILES: *c1cc(*)on1]

In some embodiments, disclosed herein is a compound of Formula I, or a pharmaceutically acceptable salt thereof, wherein A is

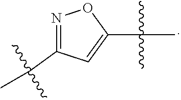 [SMILES: *c1cc(*)on1]

In some embodiments, disclosed herein is a compound of Formula I, or a pharmaceutically acceptable salt thereof, which is a compound of Formula Ib-1:

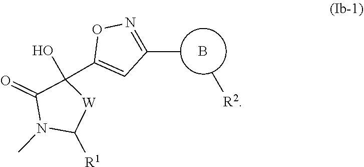

In some embodiments, disclosed herein is a compound of Formula I, or a pharmaceutically acceptable salt thereof, which is a compound of Formula Ib-2:

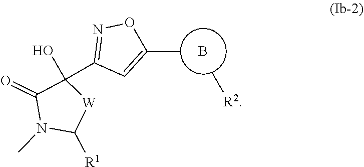

In some embodiments, disclosed herein is a compound of Formula I, or a pharmaceutically acceptable salt thereof, wherein W is CH₂, CHD, CD₂, or CF₂. In some embodiments, disclosed herein is a compound of Formula I, or a pharmaceutically acceptable salt thereof, wherein W is CH₂ or CF₂. In some embodiments, disclosed herein is a compound of Formula I, or a pharmaceutically acceptable salt thereof, wherein W is CH₂. In some embodiments, disclosed herein is a compound of Formula I, or a pharmaceutically acceptable salt thereof, wherein W is CF₂.

In some embodiments, disclosed herein is a compound of Formula I, or a pharmaceutically acceptable salt thereof, wherein B is

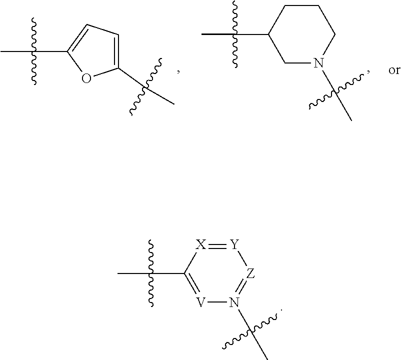

In some embodiments, disclosed herein is a compound of Formula I, or a pharmaceutically acceptable salt thereof, wherein B is

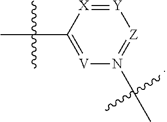

In some embodiments, disclosed herein is a compound of Formula I, or a pharmaceutically acceptable salt thereof, wherein V is N, C—H, or C—F. In some embodiments, disclosed herein is a compound of Formula I, or a pharmaceutically acceptable salt thereof, wherein V is N. In some embodiments, disclosed herein is a compound of Formula I, or a pharmaceutically acceptable salt thereof, wherein V is C—H. In some embodiments, disclosed herein is a compound of Formula I, or a pharmaceutically acceptable salt thereof, wherein V is C—R^{V}. In some embodiments, disclosed herein is a compound of Formula I, or a pharmaceutically acceptable salt thereof, wherein V is C—F.

In some embodiments, disclosed herein is a compound of Formula I, or a pharmaceutically acceptable salt thereof, wherein X is N, C—H, C—F, or C—CH₃. In some embodiments, disclosed herein is a compound of Formula I, or a pharmaceutically acceptable salt thereof, wherein X is C—H, C—F, or C—CH₃. In some embodiments, disclosed herein is a compound of Formula I, or a pharmaceutically acceptable salt thereof, wherein X is C—H or C—R^{X}. In some embodiments, disclosed herein is a compound of Formula I, or a pharmaceutically acceptable salt thereof, wherein X is C—H. In some embodiments, disclosed herein is a compound of Formula I, or a pharmaceutically acceptable salt thereof, wherein X is C—R^{X}. In some embodiments, disclosed herein is a compound of Formula I, or a pharmaceutically acceptable salt thereof, wherein X is C—F. In some embodiments, disclosed herein is a compound of Formula I, or a pharmaceutically acceptable salt thereof, wherein X is C—CH₃.

In some embodiments, disclosed herein is a compound of Formula I, or a pharmaceutically acceptable salt thereof, wherein Y is N, C—H, C—F, C—CH₃, or C—OCH₃.

In some embodiments, disclosed herein is a compound of Formula I, or a pharmaceutically acceptable salt thereof, wherein Y is C—H, C—F, C—CH₃, or C—OCH₃. In some embodiments, disclosed herein is a compound of Formula I, or a pharmaceutically acceptable salt thereof, wherein Y is C—H or C—R^{Y}. In some embodiments, disclosed herein is a compound of Formula I, or a pharmaceutically acceptable salt thereof, wherein Y is C—H. In some embodiments, disclosed herein is a compound of Formula I, or a pharmaceutically acceptable salt thereof, wherein Y is C—R^{Y}. In some embodiments, disclosed herein is a compound of Formula I, or a pharmaceutically acceptable salt thereof, wherein Y is C—F. In some embodiments, disclosed herein is a compound of Formula I, or a pharmaceutically acceptable salt thereof, wherein Y is C—CH₃. In some embodiments, disclosed herein is a compound of Formula I, or a pharmaceutically acceptable salt thereof, wherein Y is C—OCH₃.

In some embodiments, disclosed herein is a compound of Formula I, or a pharmaceutically acceptable salt thereof, wherein Z is N, C—H, C—F, or C—CH₃. In some embodiments, disclosed herein is a compound of Formula I, or a pharmaceutically acceptable salt thereof, wherein Z is N or C—H. In some embodiments, disclosed herein is a compound of Formula I, or a pharmaceutically acceptable salt thereof, wherein Z is N. In some embodiments, disclosed herein is a compound of Formula I, or a pharmaceutically acceptable salt thereof, wherein Z is C—H. In some embodiments, disclosed herein is a compound of Formula I, or a pharmaceutically acceptable salt thereof, wherein Z is C—R^{Z}. In some embodiments, disclosed herein is a compound of Formula I, or a pharmaceutically acceptable salt thereof, wherein Z is C—F. In some embodiments, disclosed herein is a compound of Formula I, or a pharmaceutically acceptable salt thereof, wherein Z is C—CH₃.

In some embodiments, disclosed herein is a compound of Formula I, or a pharmaceutically acceptable salt thereof, wherein V is C—R^{V} and R^{V} is halo, and wherein X, Y and Z are C—H. In some embodiments, disclosed herein is a compound of Formula I, or a pharmaceutically acceptable salt thereof, wherein V is C—H; X, Y and Z are N or C—H, and at least one of X, Y, and Z is N. In some embodiments, disclosed herein is a compound of Formula I, or a pharmaceutically acceptable salt thereof, wherein V is C—H, and X, Y, Z are independently selected from N, C—H, C-(halogen), C—(C₁-4 alkyl), or C—(OC₍₁₋₄₎alkyl).

In some embodiments, disclosed herein is a compound of Formula I, or a pharmaceutically acceptable salt thereof, wherein V is N or C—H; X is N or C—H; Y is N or C—H; and Z is N, C—H, or C—R^{Z}. In some embodiments, disclosed herein is a compound of Formula I, or a pharmaceutically acceptable salt thereof, wherein V is N or C—H; X is N or C—H; Y is N, C—H, or C—R^{Y}; and Z is N or C—H. In some embodiments, disclosed herein is a compound of Formula I, or a pharmaceutically acceptable salt thereof, wherein V is N or C—H; X is N or C—H; Y is N or C—H; and Z is N or C—H; wherein that no more than one of V, X, Y, and Z are N.

In some embodiments, disclosed herein is a compound of Formula I, or a pharmaceutically acceptable salt thereof, wherein R^{V}, R^{X}, R^{Y}, and R^{Z} are each, independently, halo, —C₍₁₋₄₎alkyl, or —OC₍₁₋₄₎alkyl. In some embodiments, disclosed herein is a compound of Formula I, or a pharmaceutically acceptable salt thereof, wherein R^{V}, R^{X}, R^{Y}, and R^{Z} are each, independently, halo or —C₍₁₋₄₎alkyl. In some embodiments, disclosed herein is a compound of Formula I, or a pharmaceutically acceptable salt thereof, wherein R^{V}, R^{X}, R^{Y}, and R^{Z} are each, independently, fluorine, —CH₃, or —OCH₃. In some embodiments, disclosed herein is a compound of Formula I, or a pharmaceutically acceptable salt thereof, wherein R^{V}, R^{X}, R^{Y}, and R^{Z} are fluorine.

In some embodiments, disclosed herein is a compound of Formula I, or a pharmaceutically acceptable salt thereof, wherein R^{V} is halo. In some embodiments, disclosed herein is a compound of Formula I, or a pharmaceutically acceptable salt thereof, wherein R^{V} is fluorine.

In some embodiments, disclosed herein is a compound of Formula I, or a pharmaceutically acceptable salt thereof, wherein R^{X} is halo or —C₍₁₋₄₎alkyl. In some embodiments, disclosed herein is a compound of Formula I, or a pharmaceutically acceptable salt thereof, wherein R^{X} is fluorine or —CH₃.

In some embodiments, disclosed herein is a compound of Formula I, or a pharmaceutically acceptable salt thereof, wherein R^{Y} is halo, —C₍₁₋₄₎alkyl, or —OC₍₁₋₄₎alkyl.

In some embodiments, disclosed herein is a compound of Formula I, or a pharmaceutically acceptable salt thereof, wherein R^{Y} is fluorine, —CH₃, or —OCH₃.

In some embodiments, disclosed herein is a compound of Formula I, or a pharmaceutically acceptable salt thereof, wherein R^{Z} is halo or —C₍₁₋₄₎alkyl. In some embodiments, disclosed herein is a compound of Formula I, or a pharmaceutically acceptable salt thereof, wherein R^{Z} is fluorine or —CH₃.

In some embodiments, disclosed herein is a compound of Formula I, or a pharmaceutically acceptable salt thereof, wherein: V is N, C—H, or C—F; X is N, C—H, C—F, or C—CH₃; Y is N, C—H, C—F, C—CH₃, or C—OCH₃; and Z is N, C—H, C—F, or C—CH₃. In some embodiments, disclosed herein is a compound of Formula I, or a pharmaceutically acceptable salt thereof, wherein: V is N, C—H, or C—F; X is C—H, C—F, or C—CH₃; Y is C—H, C—F, C—CH₃, or C—OCH₃; and Z is N, C—H, C—F, or C—CH₃.

In some embodiments, disclosed herein is a compound of Formula I, or a pharmaceutically acceptable salt thereof, wherein B is

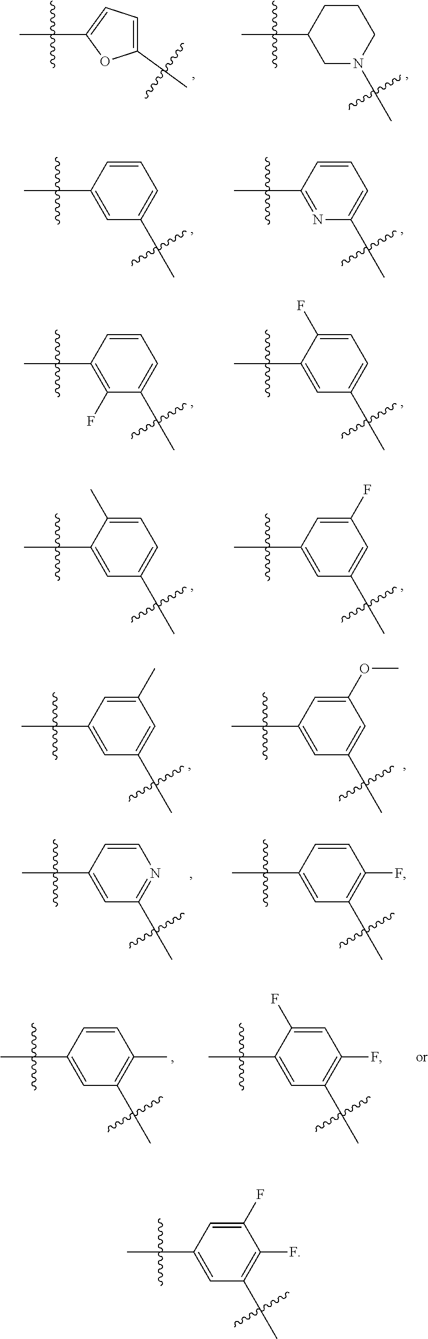

In some embodiments, disclosed herein is a compound of Formula I, or a pharmaceutically acceptable salt thereof, wherein B is

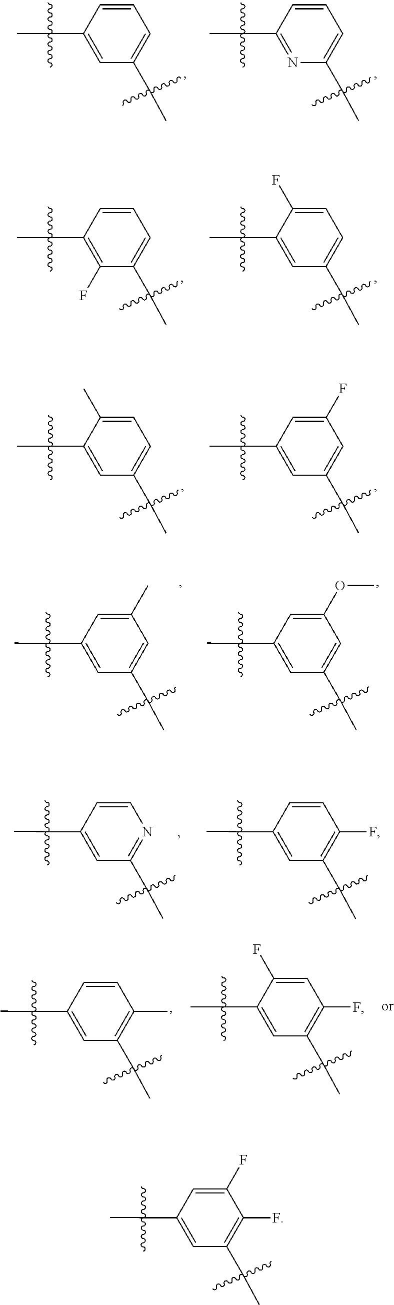

In some embodiments, disclosed herein is a compound of Formula I, or a pharmaceutically acceptable salt thereof, wherein B is

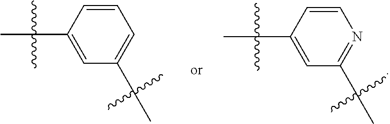 [SMILES: *c1cccc(*)c1 ; *c1ccnc(*)c1]

In some embodiments, disclosed herein is a compound of Formula I, or a pharmaceutically acceptable salt thereof, wherein B is

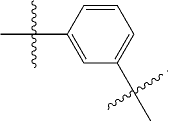 [SMILES: *c1cccc(*)c1]

In some embodiments, disclosed herein is a compound of Formula I, or a pharmaceutically acceptable salt thereof, wherein B is

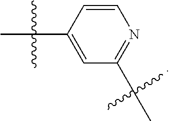 [SMILES: *c1ccnc(*)c1]

In some embodiments, disclosed herein is a compound of Formula I, or a pharmaceutically acceptable salt thereof, which is a compound of Formula Ic-1:

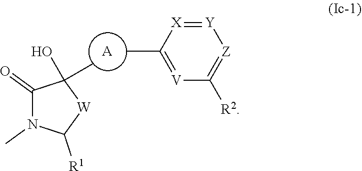

In some embodiments, disclosed herein is a compound of Formula I, or a pharmaceutically acceptable salt thereof, which is a compound of Formula Ic-2:

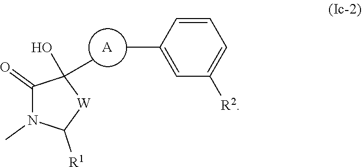

In some embodiments, disclosed herein is a compound of Formula I, or a pharmaceutically acceptable salt thereof, which is a compound of Formula Ic-3:

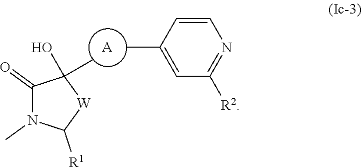

In some embodiments, disclosed herein is a compound of Formula I, or a pharmaceutically acceptable salt thereof, wherein R¹ is hydrogen, —C₍₁₋₄₎alkyl, or —C₍₁₋₄₎haloalkyl. In some embodiments, disclosed herein is a compound of Formula I, or a pharmaceutically acceptable salt thereof, wherein R¹ is hydrogen or —C₍₁₋₄₎haloalkyl. In some embodiments, disclosed herein is a compound of Formula I, or a pharmaceutically acceptable salt thereof, wherein R¹ is hydrogen or —CF₃. In some embodiments, disclosed herein is a compound of Formula I, or a pharmaceutically acceptable salt thereof, wherein R¹ is hydrogen. In some embodiments, disclosed herein is a compound of Formula I, or a pharmaceutically acceptable salt thereof, wherein R¹ is —CF₃.

In some embodiments, disclosed herein is a compound of Formula I, or a pharmaceutically acceptable salt thereof, which is a compound of any one of Formulas Id-1 to Id-5:

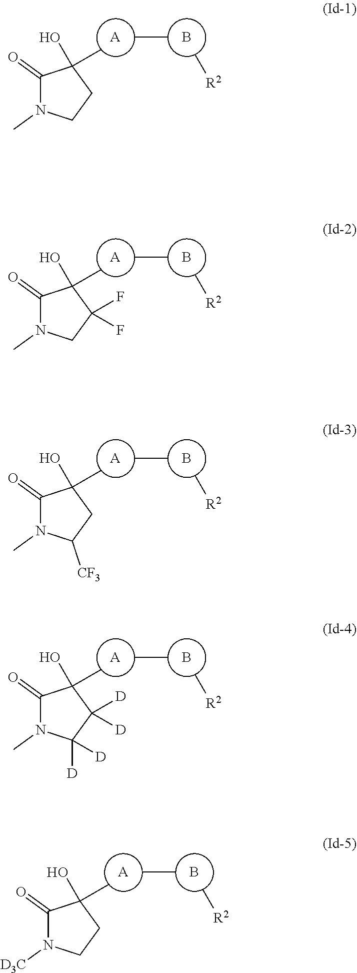

In some embodiments, disclosed herein is a compound of Formula I, or a pharmaceutically acceptable salt thereof, which is a compound of any one of Formulas Ie-1 to Ie-6:

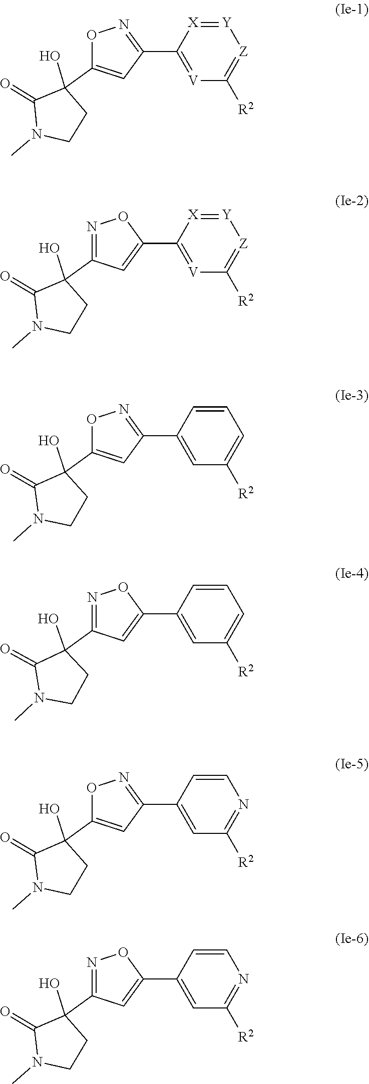

In some embodiments, disclosed herein is a compound of Formula I, or a pharmaceutically acceptable salt thereof, wherein R² is a 5- to 12-membered bi- or tricyclic ring system containing one or more heteroatoms or 5- to 10-membered heteroaryl, each of which is optionally substituted with one to five R³ groups.

In some embodiments, disclosed herein is a compound of Formula I, or a pharmaceutically acceptable salt thereof, wherein each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the —C₍₁₋₆₎alkyl, —C₍₁₋₆₎ haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎alkyl(phenyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R³ is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —C(O)N(R^{N1})(R^{N4}), or 5- to 6-membered heteroaryl, wherein the 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R³ is independently for each occurrence —N(R^{N1})(R^{N2}), —C₍₁₋₆₎alkyl, —C₍₁₋₆₎haloalkyl, —C₍₃₋₁₀₎cycloalkyl, —C(O)N(R^{N1})(R^{N4}), or 5- to 6-membered heteroaryl, wherein the 5- to 6-membered heteroaryl are optionally further substituted with one to three groups selected from —C₍₁₋₄₎alkyl, —C₍₁₋₄₎haloalkyl, —C₍₃₋₆₎cycloalkyl, and —OC₍₁₋₄₎alkyl.

In some embodiments, disclosed herein is a compound of Formula I, or a pharmaceutically acceptable salt thereof, wherein each R³ is independently for each occurrence —N(R^{N1})(R^{N2}), —C₍₁₋₆₎alkyl, —C₍₁₋₆₎haloalkyl, —C₍₃₋₁₀₎cycloalkyl, —C(O)N(R^{N1})(R^{N4}), or 5- to 6-membered heteroaryl, wherein the 5- to 6-membered heteroaryl is optionally further substituted with one to three —C₍₁₋₄₎alkyl groups.

In some embodiments, disclosed herein is a compound of Formula I, or a pharmaceutically acceptable salt thereof, wherein R² is a 5- to 12-membered bi- or tricyclic ring system containing one or more heteroatoms or a 5- to 10-membered heteroaryl, wherein the 5- to 12-membered bi- or tricyclic ring system containing one or more heteroatoms is optionally substituted with one to five R^{3c} groups and the 5- to 10-membered heteroaryl is optionally substituted with one to five R^{3d} groups.

In some embodiments, disclosed herein is a compound of Formula I, or a pharmaceutically acceptable salt thereof, wherein R² is —N(H)(5- to 10-membered heteroaryl), which is optionally substituted with one to five R^{3a} groups. In some embodiments, disclosed herein is a compound of Formula I, or a pharmaceutically acceptable salt thereof, wherein R² is —N(H)(9- to 10-membered heteroaryl), which is optionally substituted with one to five R^{3a} groups. In some embodiments, disclosed herein is a compound of Formula I, or a pharmaceutically acceptable salt thereof, wherein R² is —N(H)(thiazolopyrimidinyl), which is optionally substituted with one to five R^{3a} groups.

In some embodiments, disclosed herein is a compound of Formula I, or a pharmaceutically acceptable salt thereof, wherein each R^{3a} is independently for each occurrence halo, —N(R^{N1})(R^{N2}), —C₍₁₋₆₎alkyl, —C₍₁₋₆₎haloalkyl, —OC₍₁₋₆₎alkyl, —OC₍₁₋₆₎haloalkyl, —C₍₁₋₃₎alkyl-N(R^{N1})(R^{N3}), or —C(O)N(R^{N1})(R^{N4}). In some embodiments, disclosed herein is a compound of Formula I, or a pharmaceutically acceptable salt thereof, wherein each R^{3a} is independently for each occurrence —N(R^{N1})(R^{N2}) or —C(O)N(R^{N1})(R^{N4}). In some embodiments, disclosed herein is a compound of Formula I, or a pharmaceutically acceptable salt thereof, wherein R^{3a} is —N(R^{N1})(R^{N2}). In some embodiments, disclosed herein is a compound of Formula I, or a pharmaceutically acceptable salt thereof, wherein R^{3a} is —NH₂.

In some embodiments, disclosed herein is a compound of Formula I, or a pharmaceutically acceptable salt thereof, wherein R² is

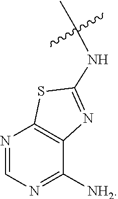 [SMILES: *Nc1nc2c(N)ncnc2s1]

In some embodiments, disclosed herein is a compound of Formula I, or a pharmaceutically acceptable salt thereof, wherein R² is —C₍₆₋₁₀₎aryl, which is optionally substituted with one to five R^{3b} groups. In some embodiments, disclosed herein is a compound of Formula I, or a pharmaceutically acceptable salt thereof, wherein R² is phenyl, which is optionally substituted with one to three R^{3b} groups.

In some embodiments, disclosed herein is a compound of Formula I, or a pharmaceutically acceptable salt thereof, wherein each R^{3b} is independently for each occurrence halo, —N(R^{N1})(R^{N2}), —C₍₁₋₆₎alkyl, —C₍₁₋₆₎haloalkyl, —OC₍₁₋₆₎alkyl, —OC₍₁₋₆₎haloalkyl, —C₍₁₋₃₎alkyl-N(R^{N1})(R^{N3}), or —C(O)N(R^{N1})(R^{N4}). In some embodiments, disclosed herein is a compound of Formula I, or a pharmaceutically acceptable salt thereof, wherein each R^{3b} is independently for each occurrence —N(R^{N1})(R^{N2}) or —C(O)N(R^{N1})(R^{N4}) In some embodiments, disclosed herein is a compound of Formula I, or a pharmaceutically acceptable salt thereof, wherein R^{3b} is —C(O)N(R^{N1})(R^{N4}). In some embodiments, disclosed herein is a compound of Formula I, or a pharmaceutically acceptable salt thereof, wherein R^{3b} is —C(O)NH₂.

In some embodiments, disclosed herein is a compound of Formula I, or a pharmaceutically acceptable salt thereof, wherein R² is

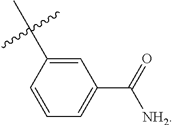 [SMILES: *c1cccc(C(N)=O)c1]

In some embodiments, disclosed herein is a compound of Formula I, or a pharmaceutically acceptable salt thereof, wherein R² is a 5- to 12-membered bi- or tricyclic ring system containing one or more heteroatoms, which is optionally substituted with one to five R^{3c} groups. In some embodiments, disclosed herein is a compound of Formula I, or a pharmaceutically acceptable salt thereof, wherein R² is a 7- to 12-membered bicyclic ring system containing one or more heteroatoms, which is optionally substituted with one to three R^{3c} groups. In some embodiments, disclosed herein is a compound of Formula I, or a pharmaceutically acceptable salt thereof, wherein R² is a 7- to 12-membered tricyclic ring system containing one or more heteroatoms, which is optionally substituted with one to three R^{3c} groups.

In some embodiments, disclosed herein is a compound of Formula I, or a pharmaceutically acceptable salt thereof, wherein R² is selected from the group consisting of:

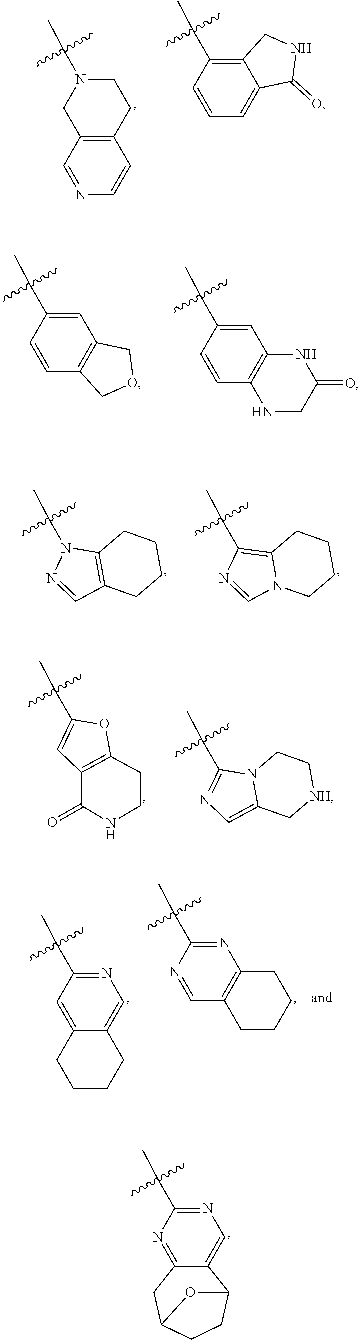

each of which is optionally substituted with one to three R^{3c} groups.

In some embodiments, disclosed herein is a compound of Formula I, or a pharmaceutically acceptable salt thereof, wherein R² is selected from the group consisting of:

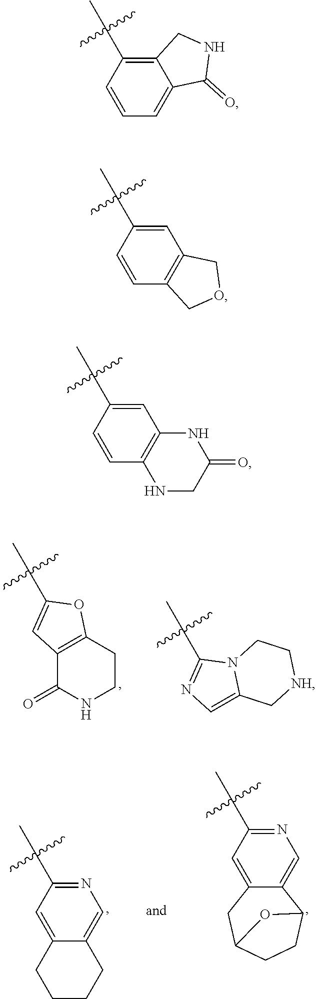

each of which is optionally substituted with one to three R^{3c} groups.

In some embodiments, disclosed herein is a compound of Formula I, or a pharmaceutically acceptable salt thereof, wherein R² is:

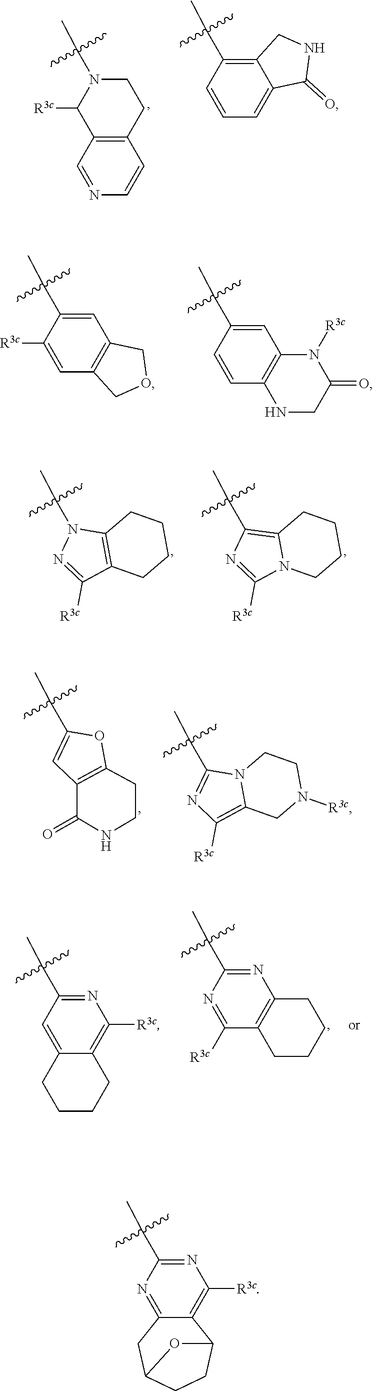

In some embodiments, disclosed herein is a compound of Formula I, or a pharmaceutically acceptable salt thereof, wherein R² is:

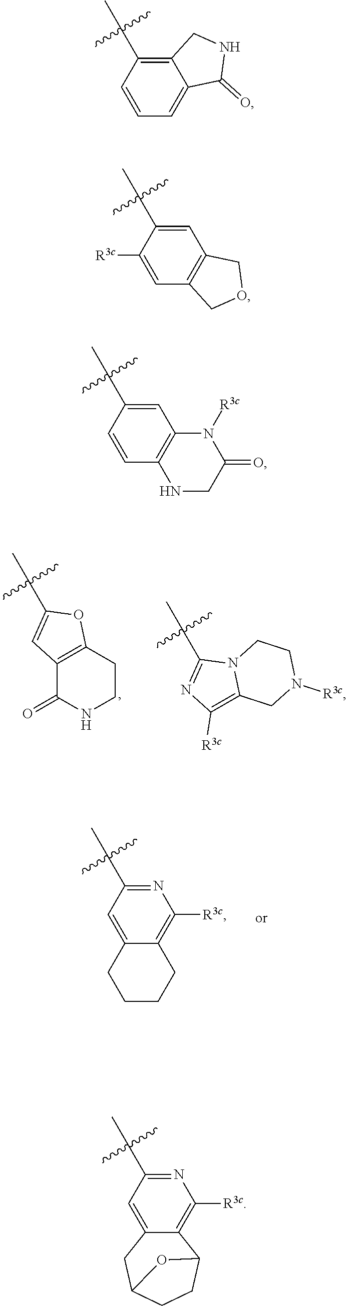

In some embodiments, disclosed herein is a compound of Formula I, or a pharmaceutically acceptable salt thereof, wherein R^{3c} is halo, —N(R^{N1})(R^{N2}), —C₍₁₋₆₎alkyl, —C₍₁₋₆₎haloalkyl, —OC₍₁₋₆₎alkyl, —OC₍₁₋₆₎haloalkyl, —C₍₁₋₃₎alkyl-N(R^{N1})(R^{N3}), —C(O)OH, —C(O)N(R^{N1})(R^{N4}), or —C₍₁₋₃₎alkyl(phenyl). In some embodiments, disclosed herein is a compound of Formula I, or a pharmaceutically acceptable salt thereof, wherein R^{3c} is —N(R^{N1})(R^{N2}), —C₍₁₋₆₎alkyl, —C₍₁₋₆₎haloalkyl, —OC₍₁₋₆₎alkyl, —C(O)OH, —C(O)N(R^{N1})(R^{N4}), or —C₍₁₋₃₎alkyl(phenyl). In some embodiments, disclosed herein is a compound of Formula I, or a pharmaceutically acceptable salt thereof, wherein R^{3c} is —NH₂, —C₍₁₋₆₎alkyl, —C₍₁₋₆₎ haloalkyl, —OC₍₁₋₆₎alkyl, —C(O)OH, —C(O)NH₂, or —C₍₁₋₃₎alkyl(phenyl).

In some embodiments, disclosed herein is a compound of Formula I, or a pharmaceutically acceptable salt thereof, wherein R² is:

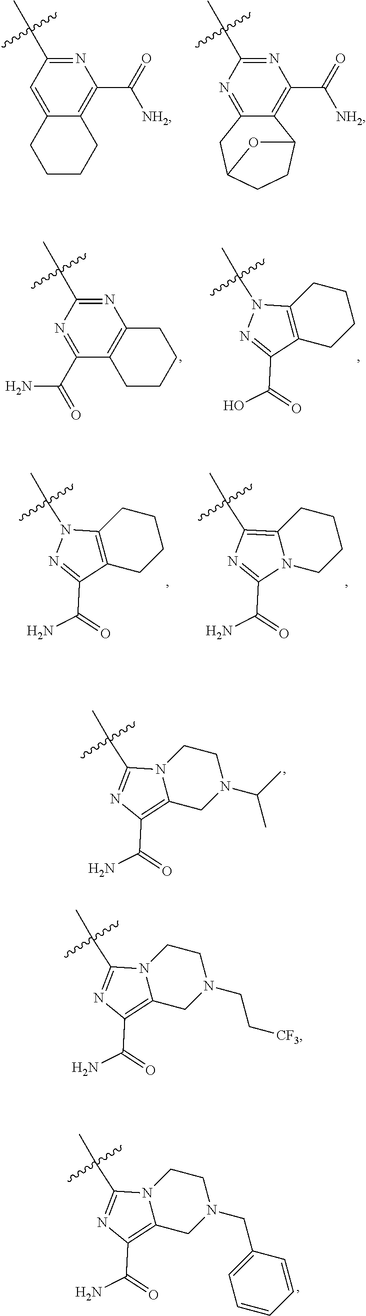

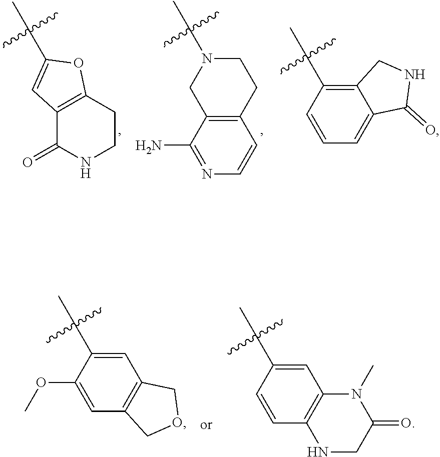

In some embodiments, disclosed herein is a compound of Formula I, or a pharmaceutically acceptable salt thereof, wherein R² is:

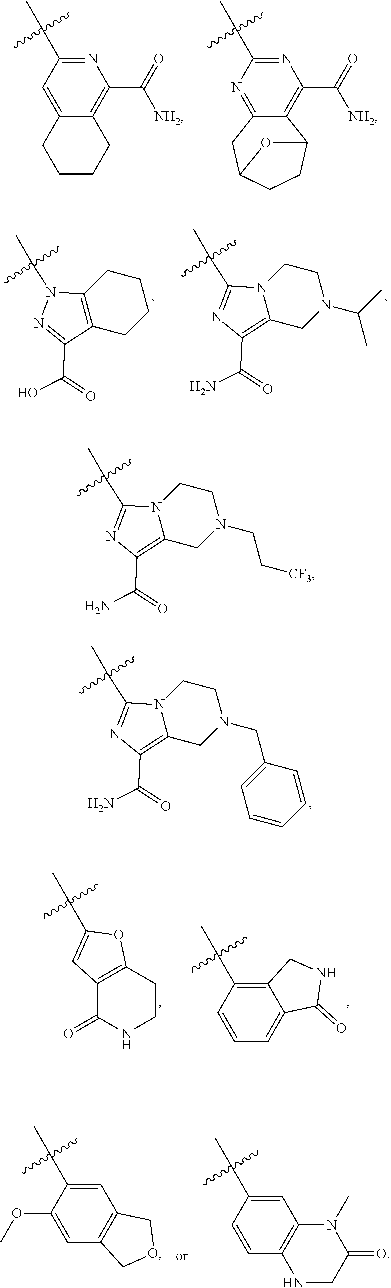

In some embodiments, disclosed herein is a compound of Formula I, or a pharmaceutically acceptable salt thereof, wherein R² is a 5- to 10-membered heteroaryl, which is optionally substituted with one to five R^{3d} groups. In some embodiments, disclosed herein is a compound of Formula I, or a pharmaceutically acceptable salt thereof, wherein R² is a 5- to 6-membered monocyclic heteroaryl or a 9- to 10-membered bicyclic heteroaryl, each of which is optionally substituted with one to five R^{3d} groups.

In some embodiments, disclosed herein is a compound of Formula I, or a pharmaceutically acceptable salt thereof, wherein R² is a 5-membered heteroaryl, which is optionally substituted with one to three R^{3d} groups. In some embodiments, disclosed herein is a compound of Formula I, or a pharmaceutically acceptable salt thereof, wherein R² is a 6-membered heteroaryl, which is optionally substituted with one to three R^{3d} groups. In some embodiments, disclosed herein is a compound of Formula I, or a pharmaceutically acceptable salt thereof, wherein R² is a 9-membered heteroaryl, which is optionally substituted with one to three R^{3d} groups. In some embodiments, disclosed herein is a compound of Formula I, or a pharmaceutically acceptable salt thereof, wherein R² is a 10-membered heteroaryl, which is optionally substituted with one to three R^{3d} groups.

In some embodiments, disclosed herein is a compound of Formula I, or a pharmaceutically acceptable salt thereof, wherein R² is pyrazolyl, imidazolyl, furanyl, thiazolyl, pyridinyl, pyrimidinyl, pyrazinyl, indolyl, indazolyl, benzimidazolyl, pyrrolopyridinyl, pyrrolopyrimidinyl, pyrrolopyrazinyl, pyrrolopyridazinyl, pyrrolotriazinyl, pyrazolopyridinyl, pyrazolopyrimidinyl, imidazopyridinyl, imidazopyrazinyl, imidazopyridazinyl, triazolopyridinyl, quinazolinyl, isoquinolinonyl, quinazolinonyl, pyridopyrimidinyl, pyridopyrimidinonyl, pyrimidopyrimidinyl, benzoxazolyl, thiazolopyrimidinyl, furopyridinyl, or thienopyrimidinyl, each of which is optionally substituted with one to three R^{3d} groups. In some embodiments, disclosed herein is a compound of Formula I, or a pharmaceutically acceptable salt thereof, wherein R² is thiazolyl, pyridinyl, pyrimidinyl, indazolyl, pyrazolopyridinyl, or imidazopyridinyl, each of which is optionally substituted with one to three R^{3d} groups.

In some embodiments, disclosed herein is a compound of Formula I, or a pharmaceutically acceptable salt thereof, wherein R² is selected from the group consisting of:

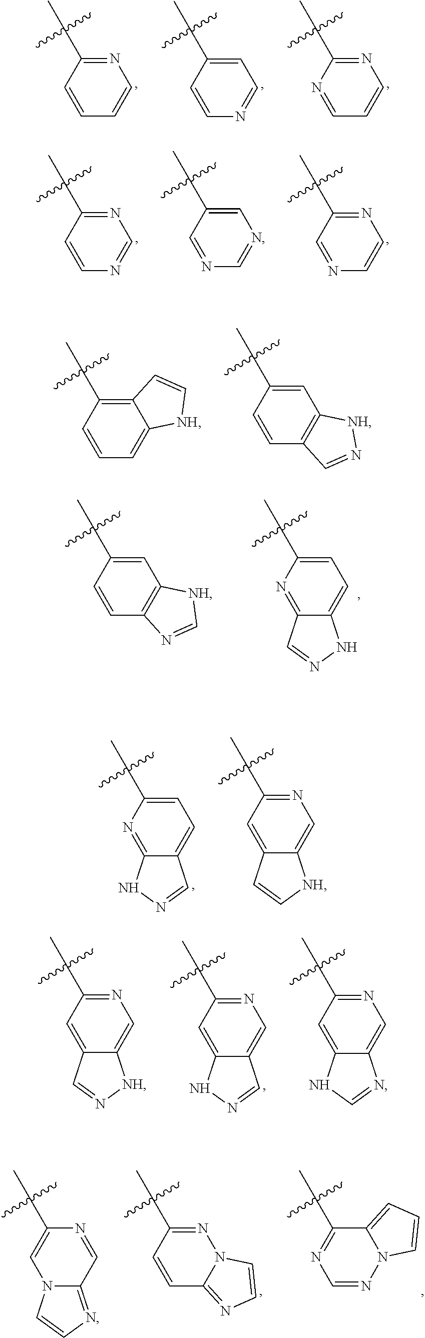

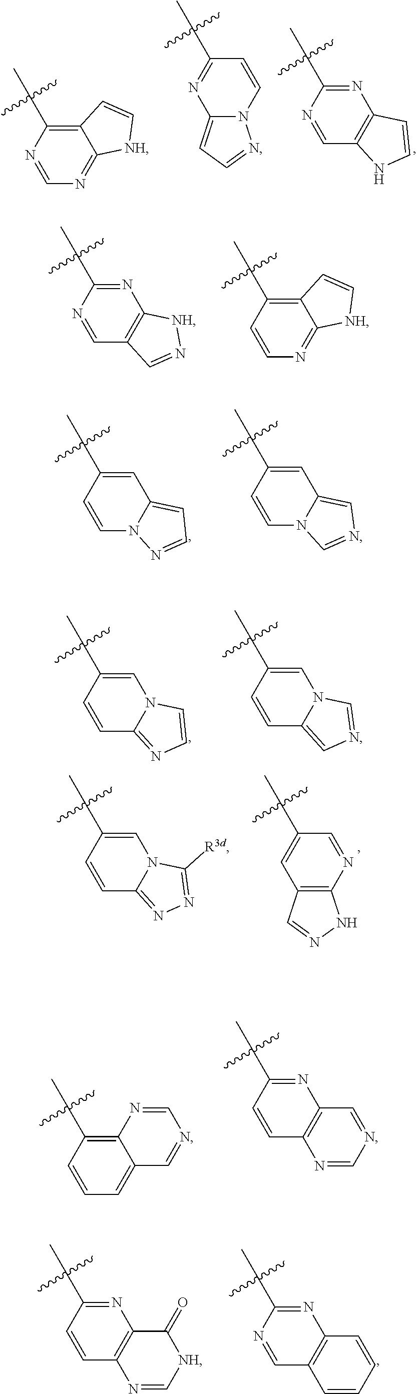

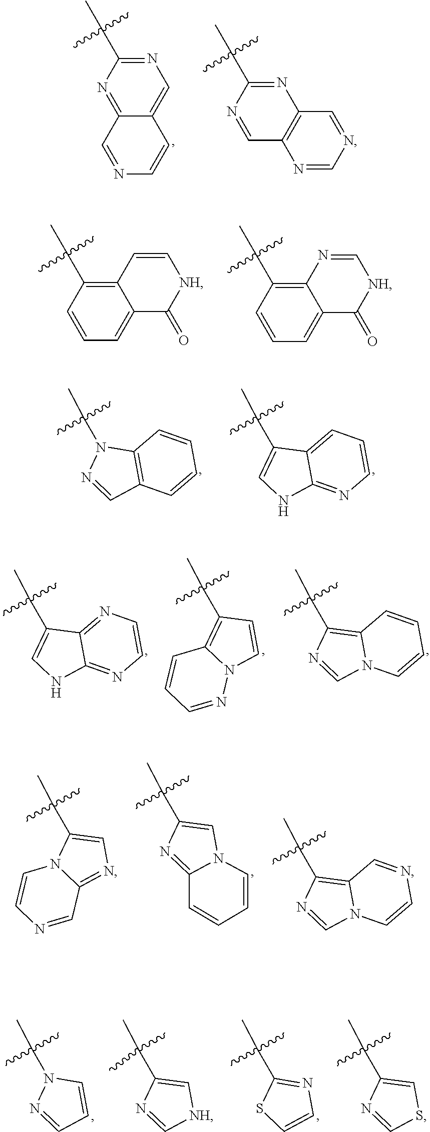

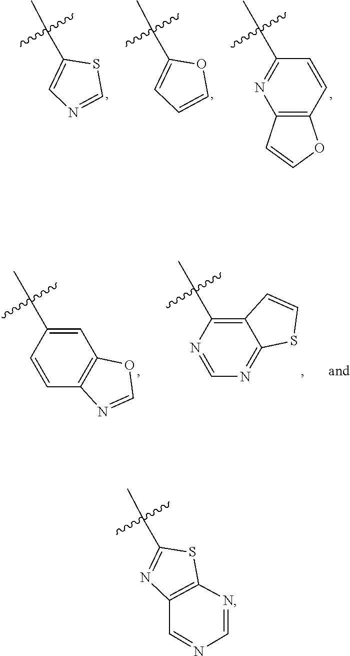 [SMILES: *c1cncs1 ; *c1ccco1 ; *c1ccc2occc2n1 ; *c1ccc2ncoc2c1 ; *c1ncnc2sccc12 ; *c1nc2cncnc2s1]

each of which is optionally substituted with one to three R^{3d} groups.

In some embodiments, disclosed herein is a compound of Formula I, or a pharmaceutically acceptable salt thereof, wherein R² is selected from the group consisting of:

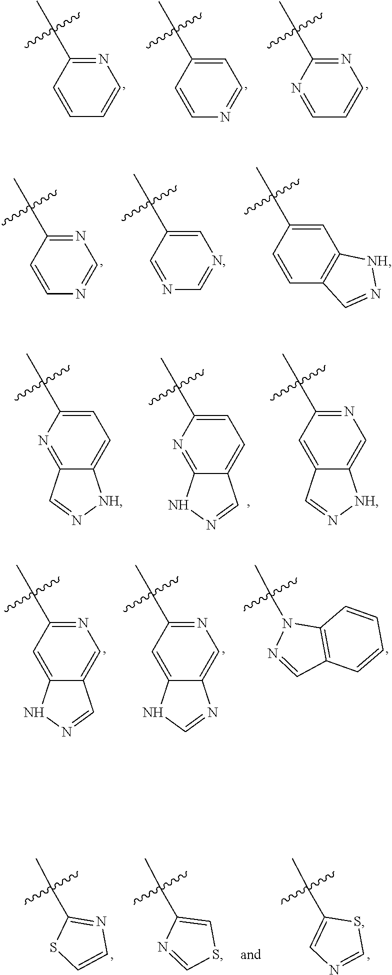

each of which is optionally substituted with one to three R^{3d} groups.

In some embodiments, disclosed herein is a compound of Formula I, or a pharmaceutically acceptable salt thereof, wherein R² is selected from the group consisting of:

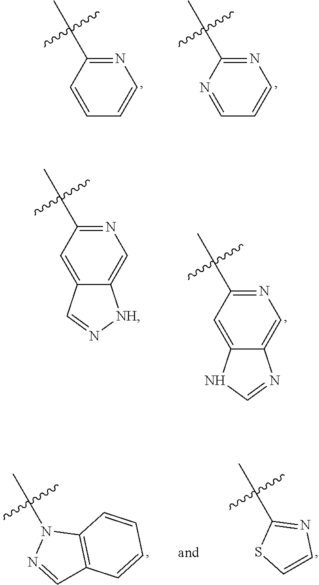 [SMILES: *c1ccccn1 ; *c1ncccn1 ; *c1cc2cn[nH]c2cn1 ; *c1cc2[nH]cnc2cn1 ; *n1ncc2ccccc21 ; *c1nccs1]

each of which is optionally substituted with one to three R^{3d} groups.

In some embodiments, disclosed herein is a compound of Formula I, or a pharmaceutically acceptable salt thereof, wherein R² is:

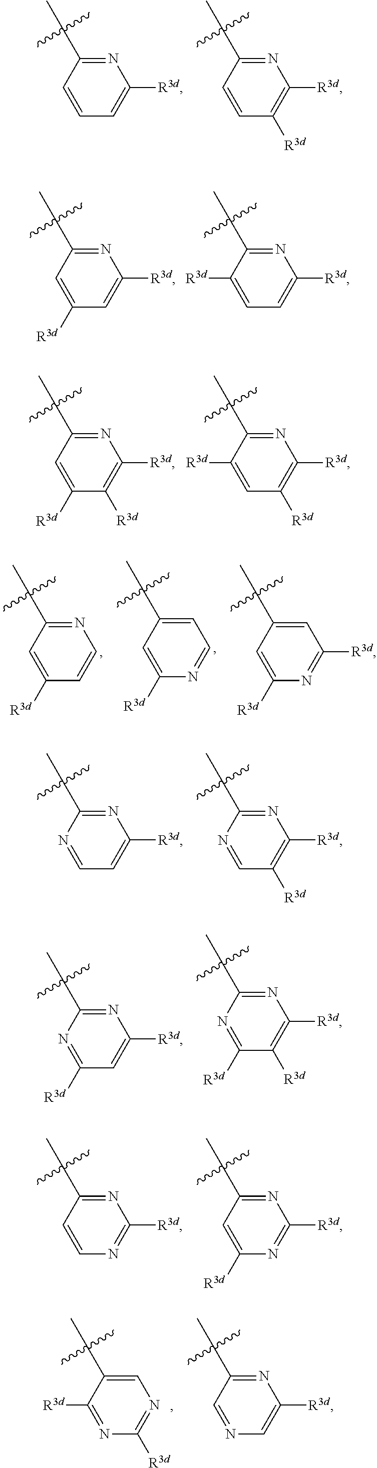

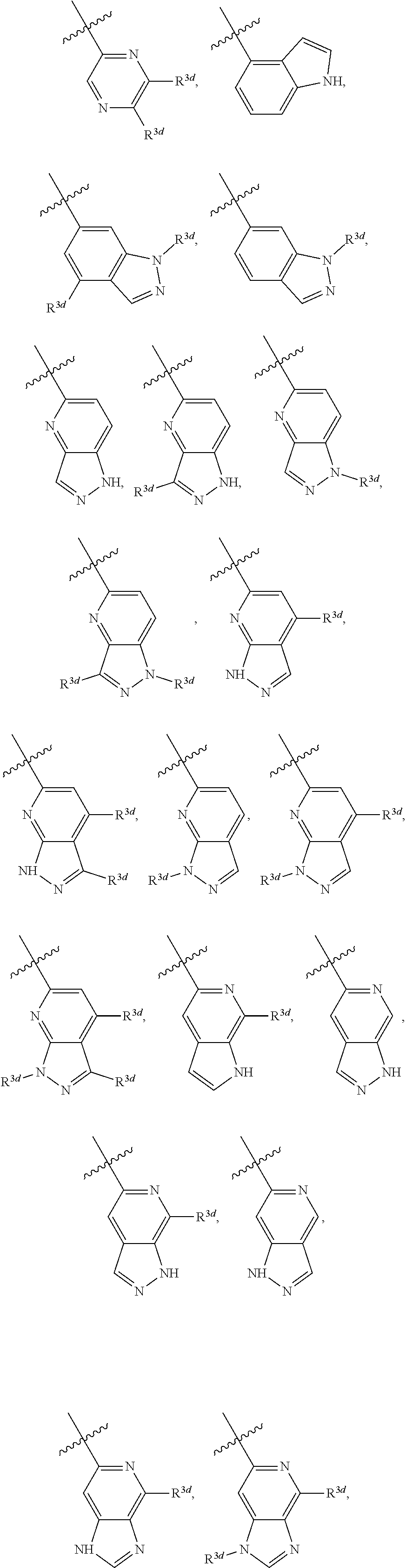

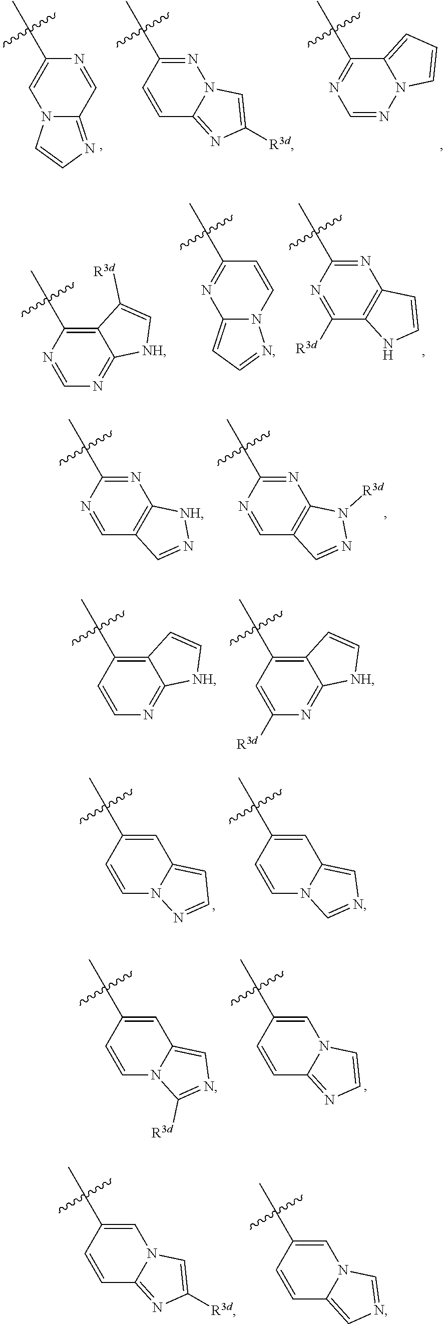

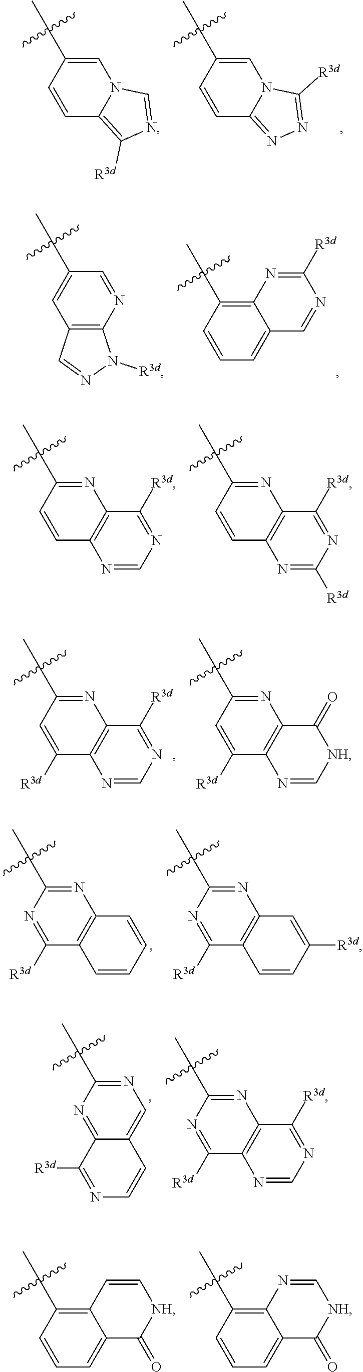

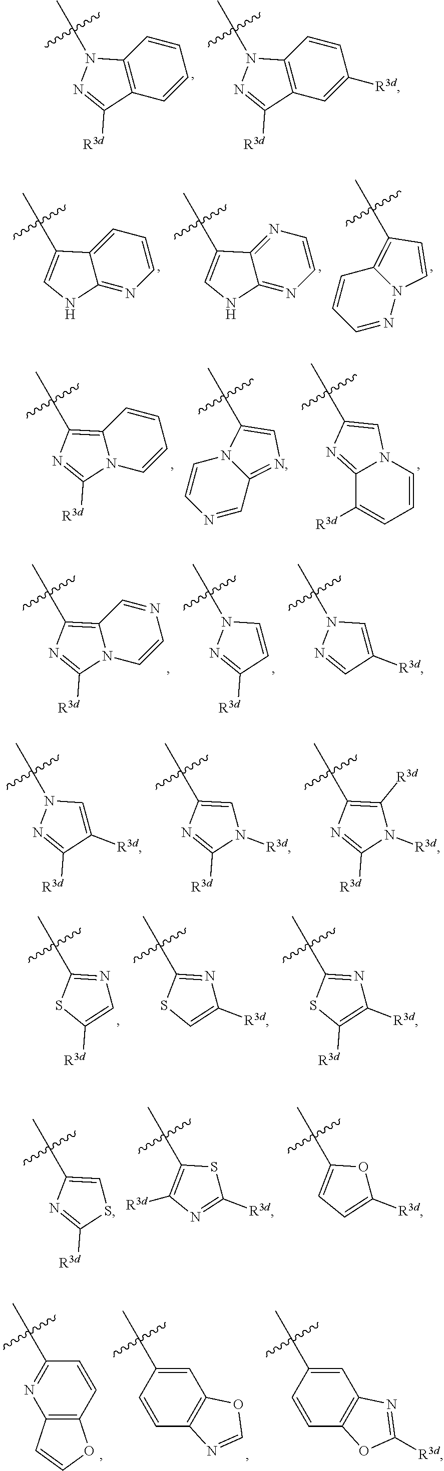

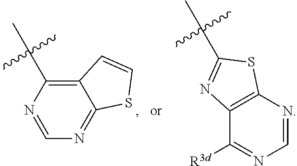

In some embodiments, disclosed herein is a compound of Formula I, or a pharmaceutically acceptable salt thereof, wherein R² is:

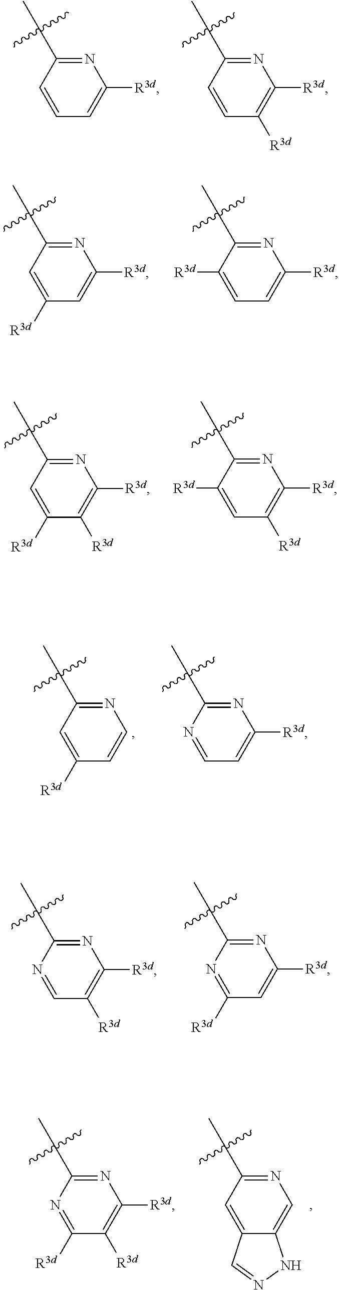

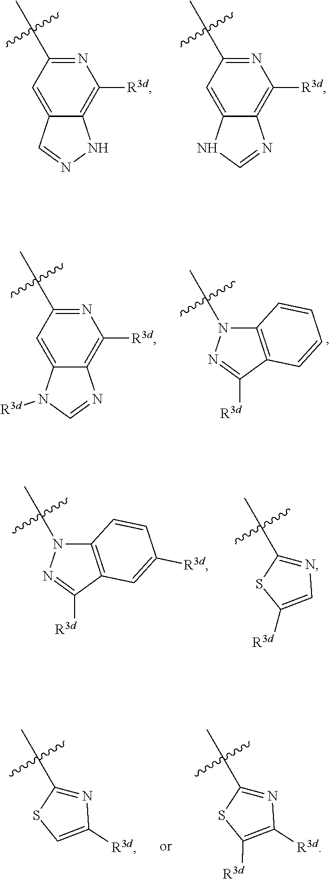

In some embodiments, disclosed herein is a compound of Formula I, or a pharmaceutically acceptable salt thereof, wherein each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycycloalkyl, —C₍₁₋₃₎alkyl-N(R^{N1})(R^{N3}), —C(O)N(R^{N1})(R^{N4}), —C₍₁₋₃₎ alkyl(3- to 8-membered heterocyclyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎ alkyl, —OC₍₁₋₆₎haloalkyl, —OC₍₃₋₁₀₎cycloalkyl, —C₍₁₋₃₎alkyl(3- to 8-membered heterocyclyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)N(R^{N1})(R^{N4}), —C₍₁₋₃₎alkyl(3- to 8-membered heterocyclyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R^{3d} is independently for each occurrence halo, —N(R^{N1})(R^{N2}), —C₍₁₋₆₎alkyl, —C₍₁₋₆₎haloalkyl, —C₍₃₋₁₀₎cycloalkyl, —OC₍₁₋₆₎alkyl, —C(O)N(R^{N1})(R^{N4}), or 5- to 6-membered heteroaryl, wherein the 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl.

In some embodiments, disclosed herein is a compound of Formula I, or a pharmaceutically acceptable salt thereof, wherein each R^{3d} is independently for each occurrence —N(R^{N1})(R^{N2}), —C₍₁₋₆₎alkyl, —C₍₁₋₆₎haloalkyl, —C₍₃₋₁₀₎cycloalkyl, —C(O)N(R^{N1})(R^{N4}), or 5- to 6-membered heteroaryl, wherein the 5- to 6-membered heteroaryl are optionally further substituted with one to three groups selected from —C₍₁₋₄₎alkyl, —C₍₁₋₄₎haloalkyl, —C₍₃₋₆₎cycloalkyl, and —OC₍₁₋₄₎alkyl.

In some embodiments, disclosed herein is a compound of Formula I, or a pharmaceutically acceptable salt thereof, wherein each R^{3d} is independently for each occurrence —N(R^{N1})(R^{N2}), —C₍₁₋₆₎alkyl, —C₍₁₋₆₎haloalkyl, —C₍₃₋₁₀₎cycloalkyl, —C(O)N(R^{N1})(R^{N4}), or 5- to 6-membered heteroaryl, wherein the 5- to 6-membered heteroaryl is optionally further substituted with one to three —C₍₁₋₄₎alkyl groups.

In some embodiments, disclosed herein is a compound of Formula I, or a pharmaceutically acceptable salt thereof, wherein R² is:

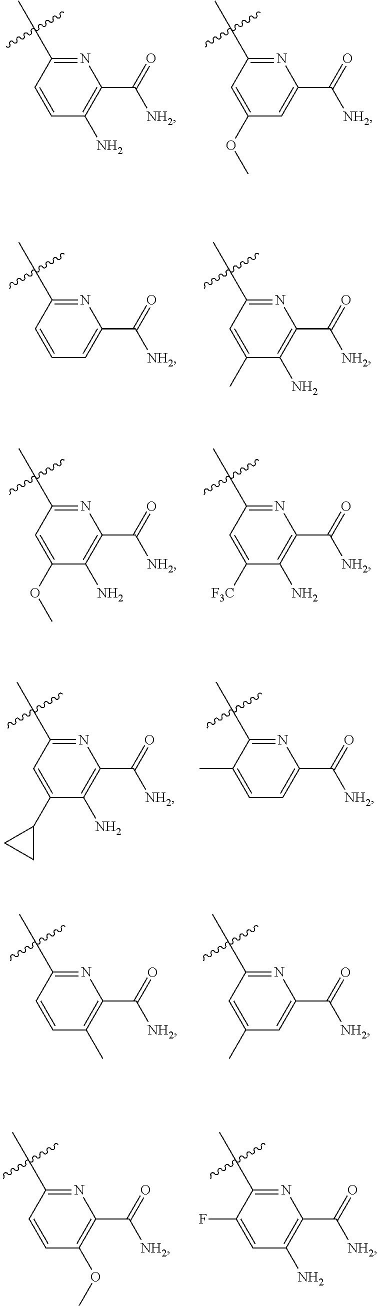

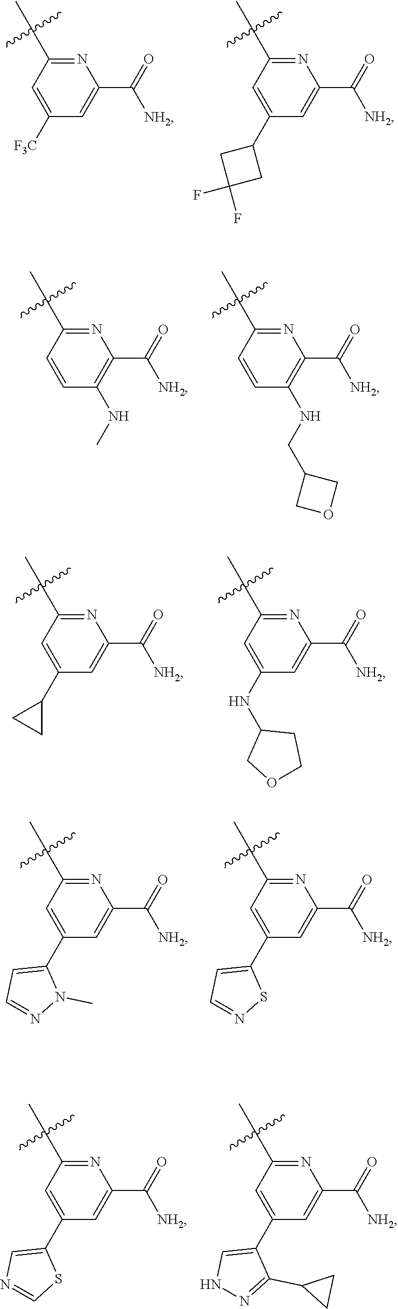

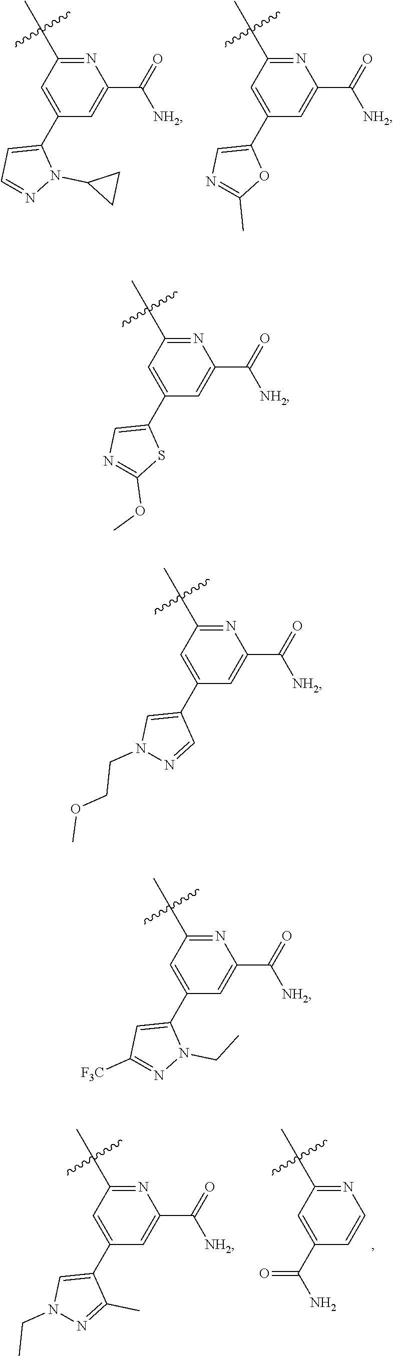

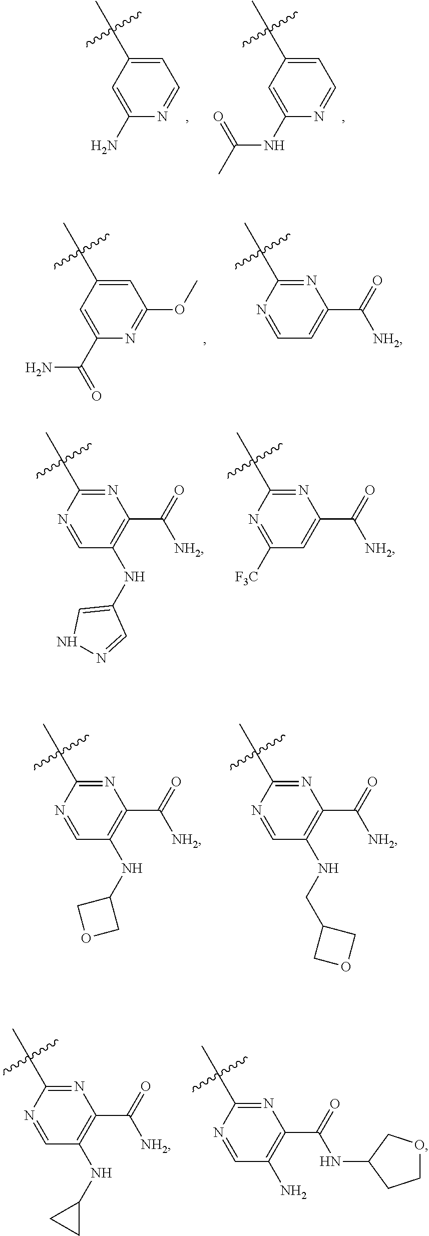

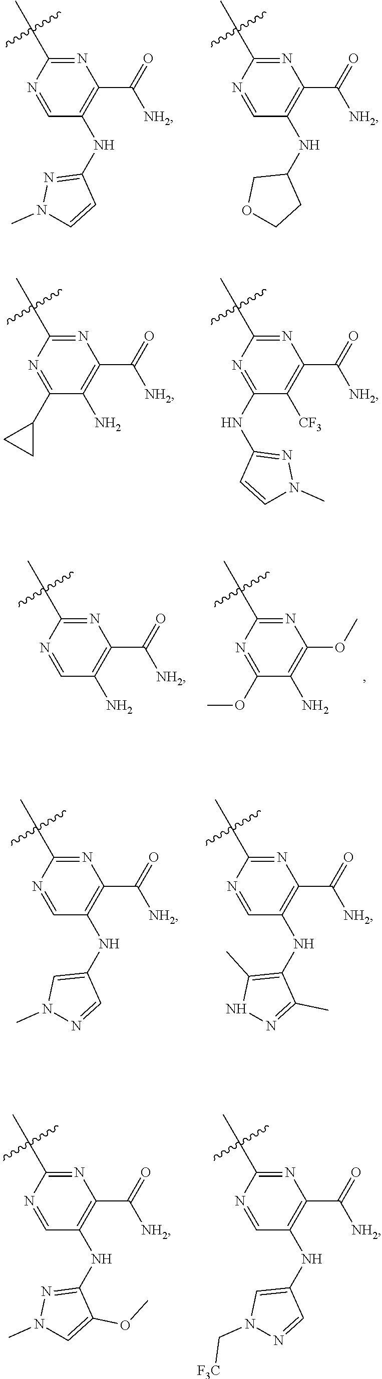

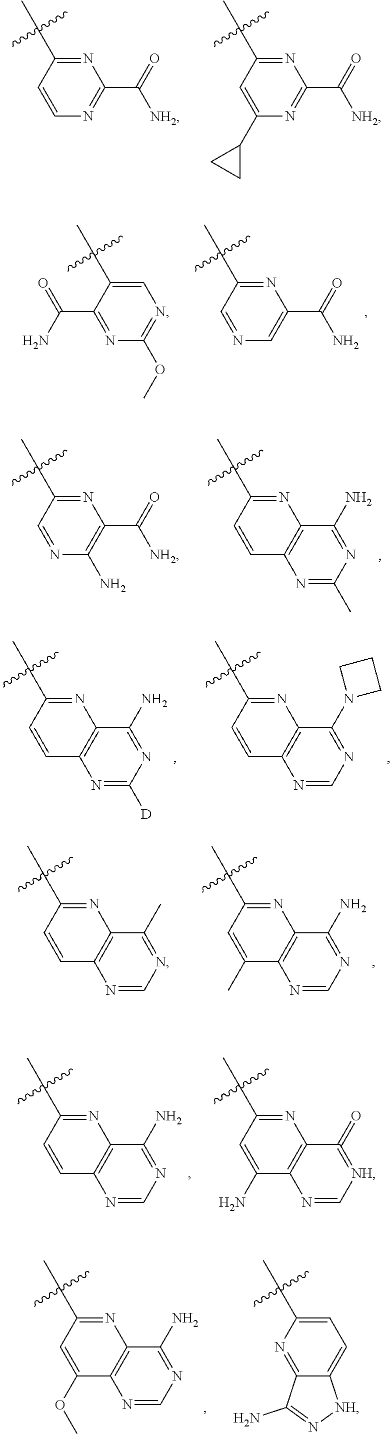

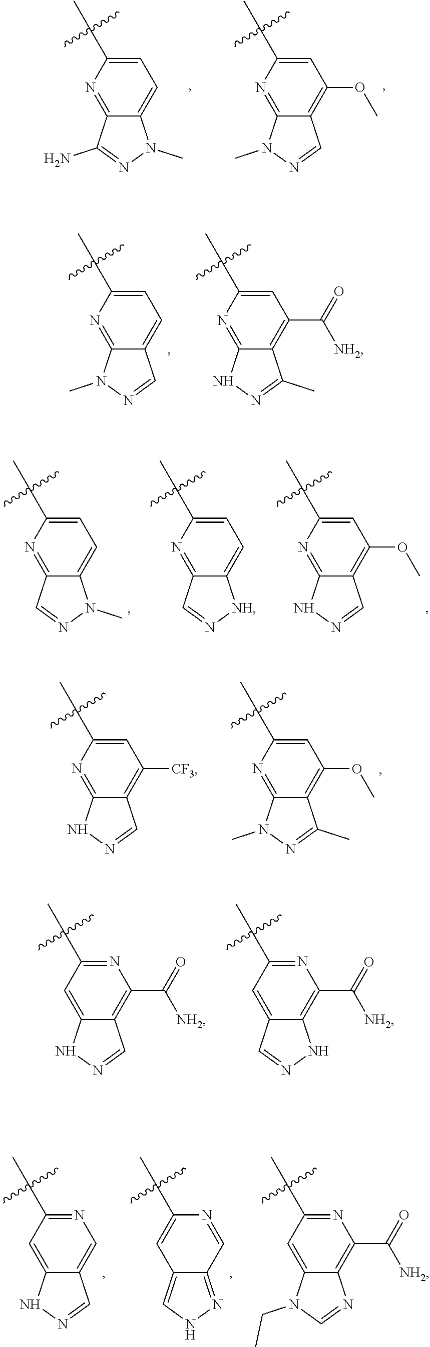

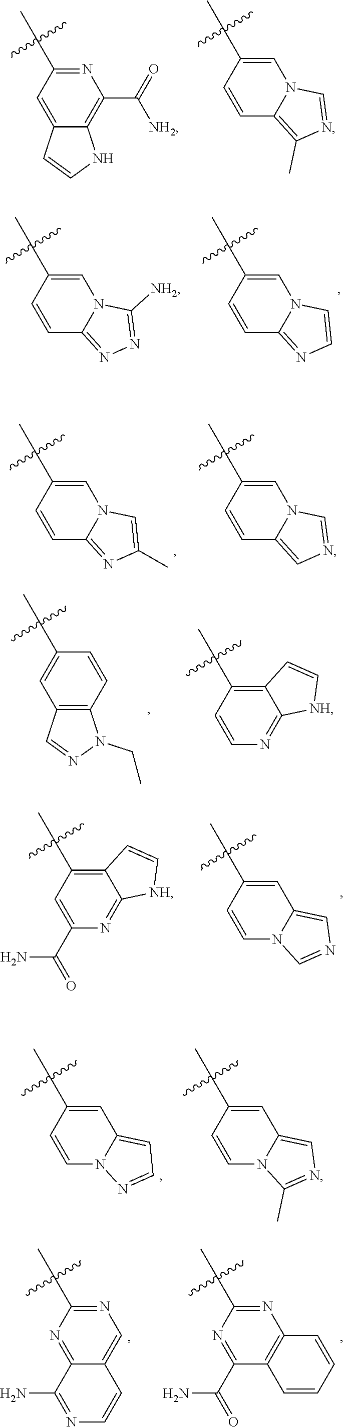

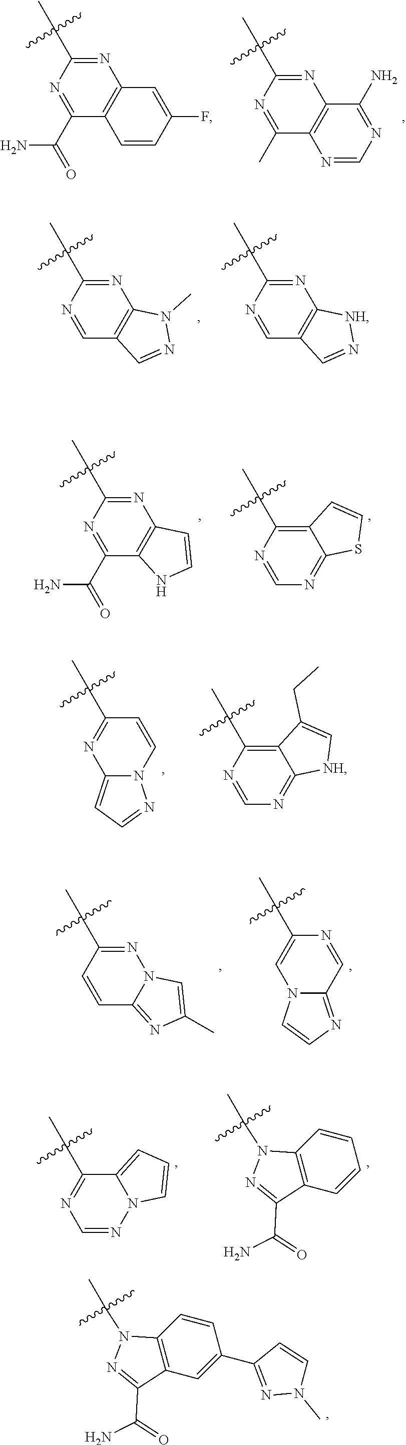

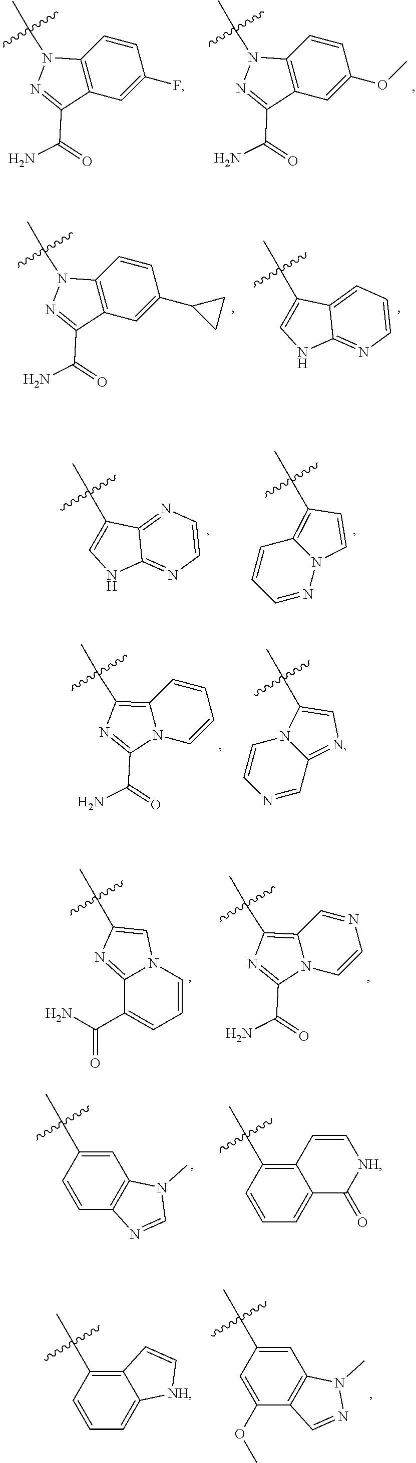

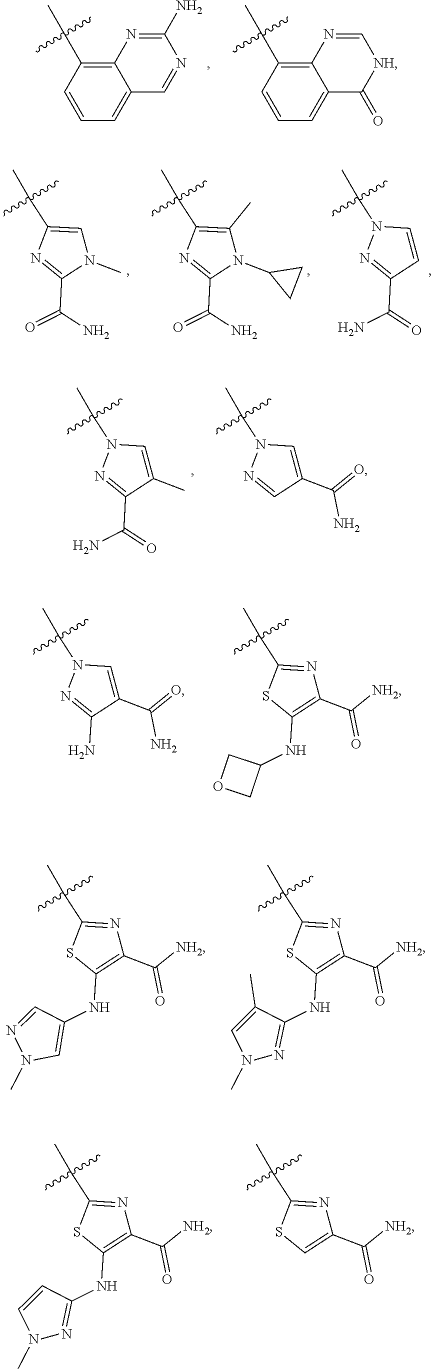

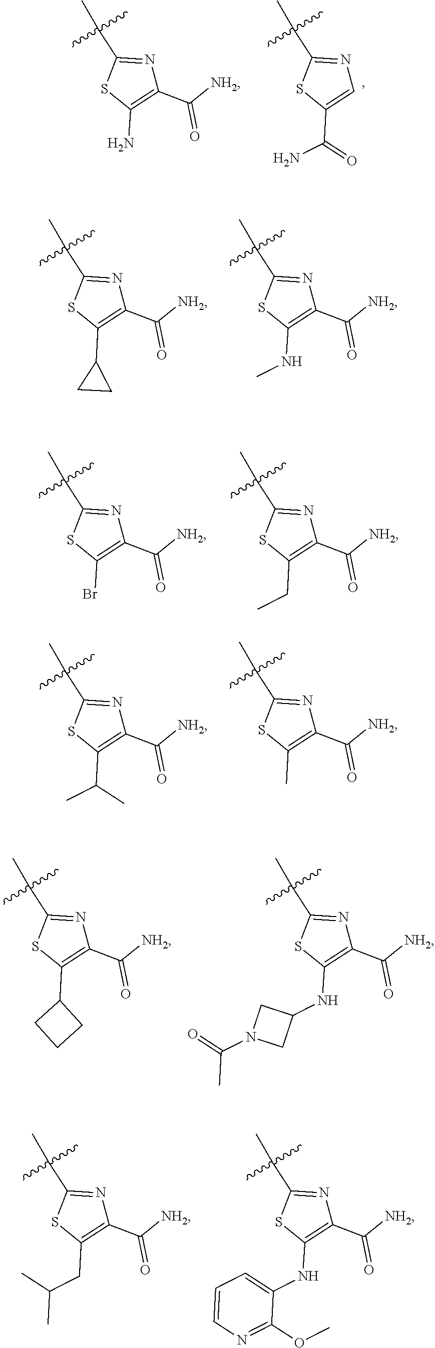

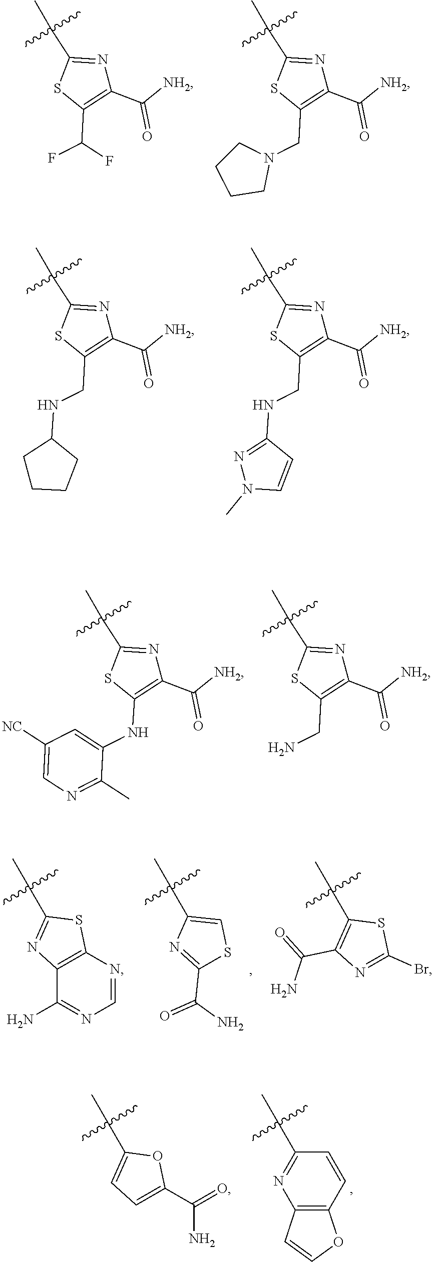

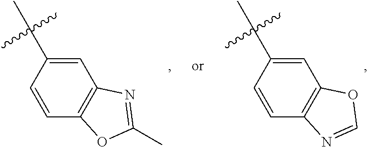 [SMILES: *c1ccc2oc(C)nc2c1 ; *c1ccc2ncoc2c1]

In some embodiments, disclosed herein is a compound of Formula I, or a pharmaceutically acceptable salt thereof, wherein R² is:

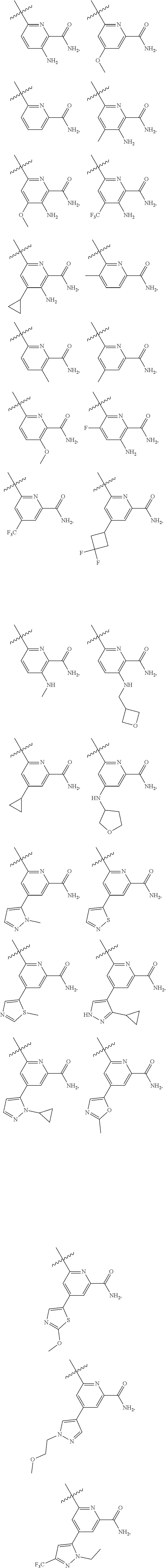

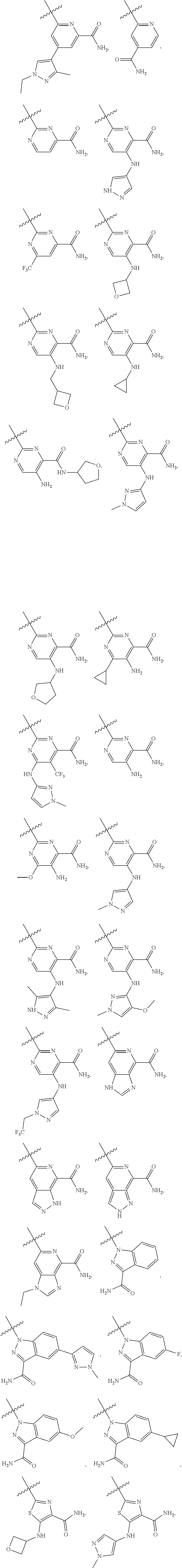

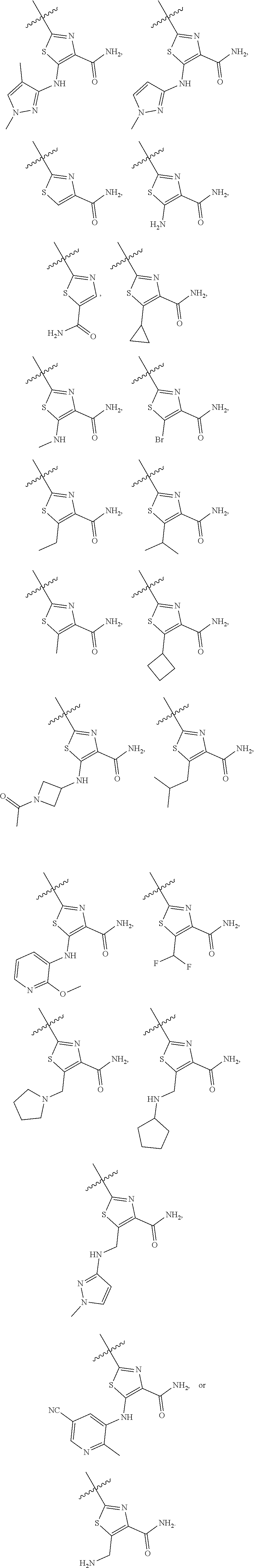

In some embodiments, disclosed herein is a compound of Formula I, or a pharmaceutically acceptable salt thereof, wherein R² is:

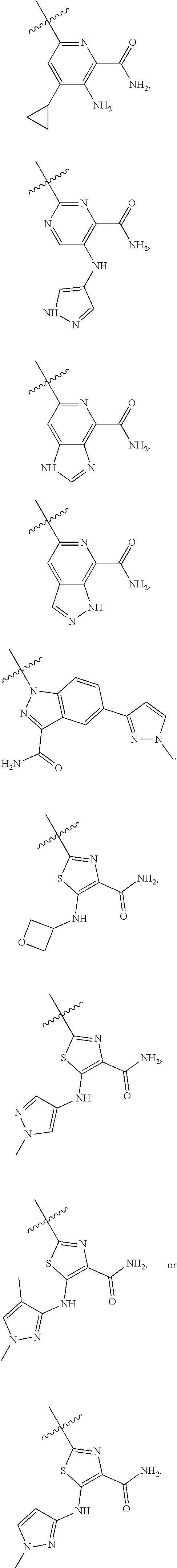

In some embodiments, disclosed herein is a compound of Formula I, or a pharmaceutically acceptable salt thereof, wherein R² is:

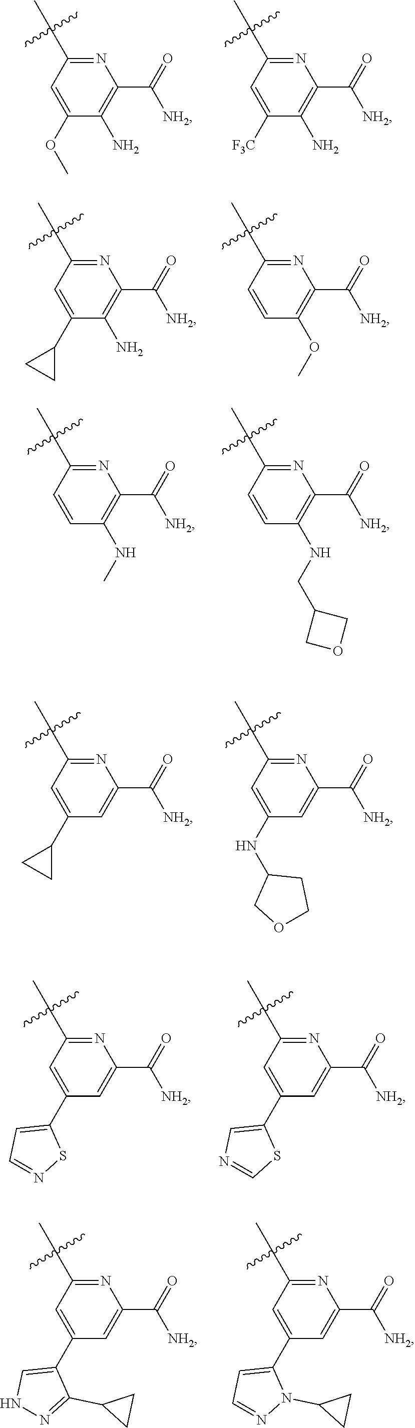

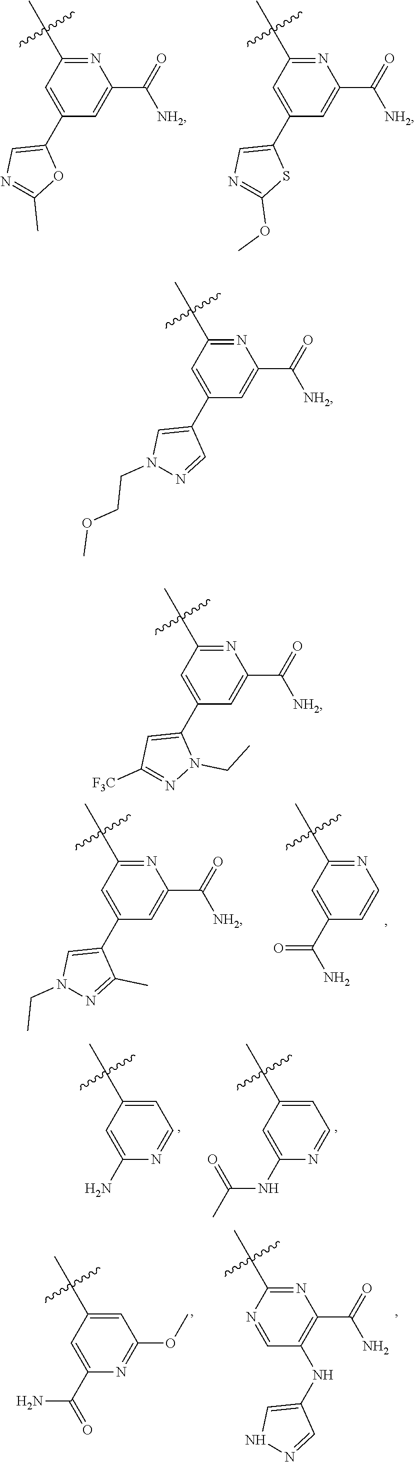

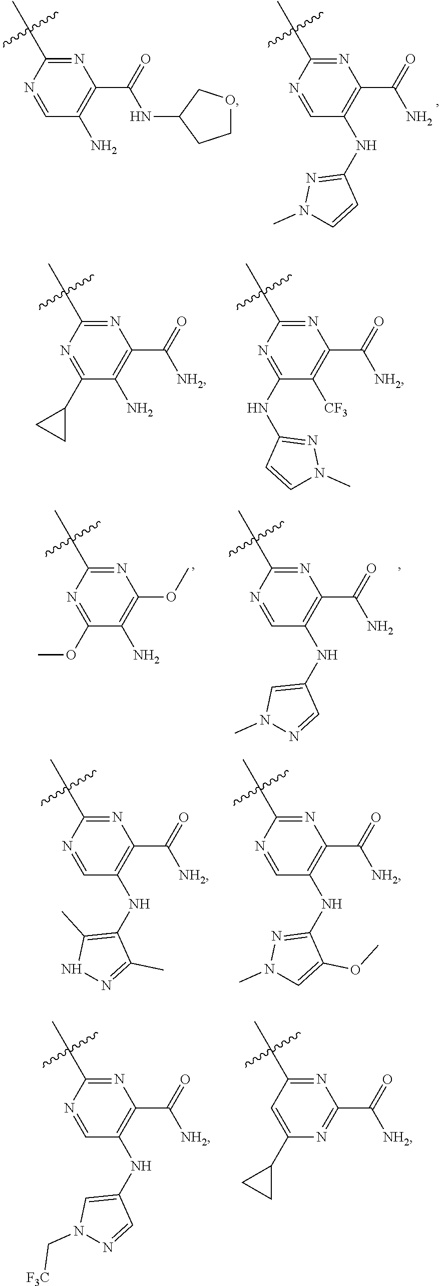

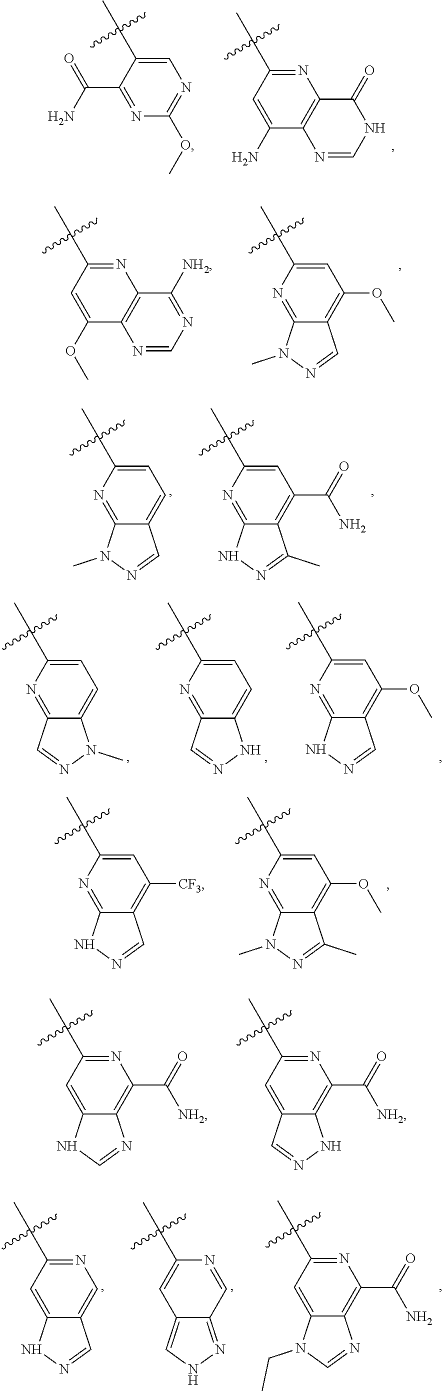

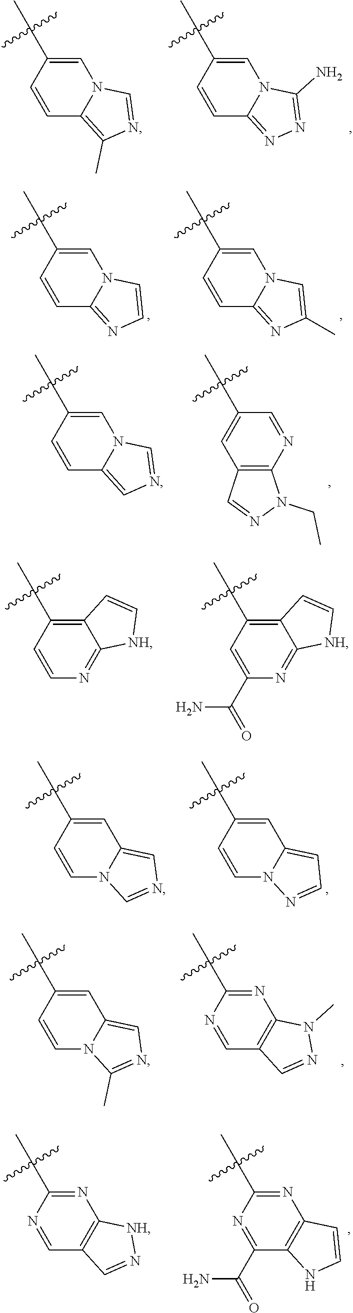

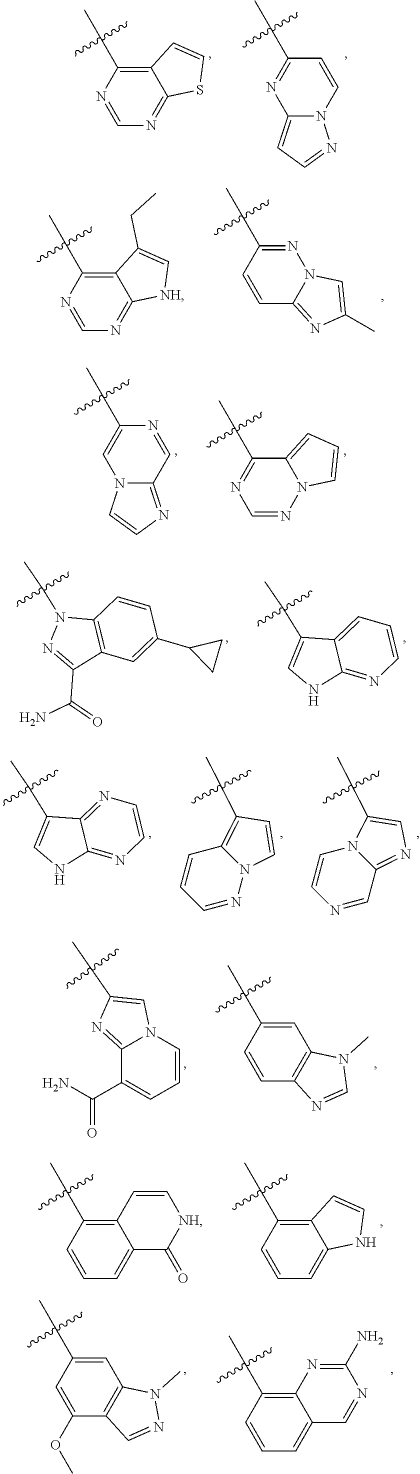

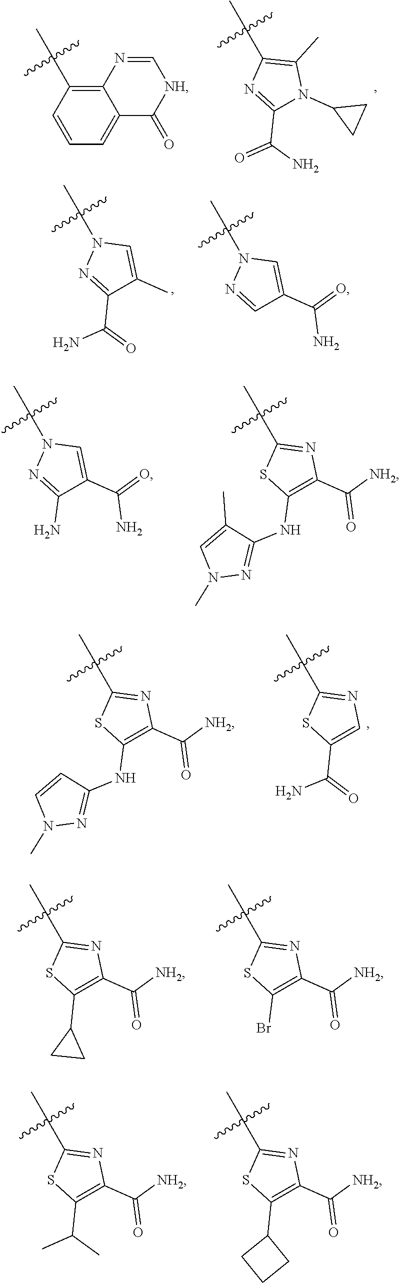

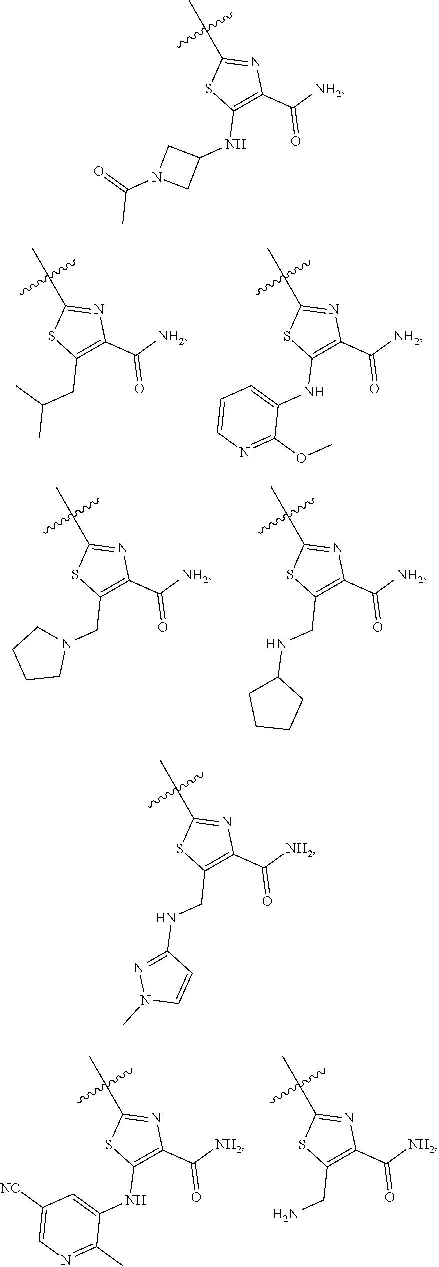

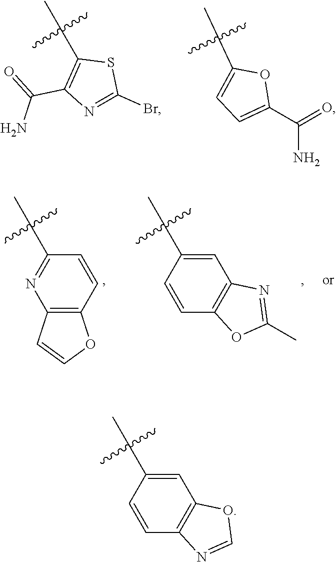 [SMILES: *c1sc(Br)nc1C(N)=O ; *c1ccc(C(N)=O)o1 ; *c1ccc2occc2n1 ; *c1ccc2oc(C)nc2c1 ; *c1ccc2ncoc2c1]

In some embodiments, disclosed herein is a compound of Formula I, or a pharmaceutically acceptable salt thereof, wherein R² is:

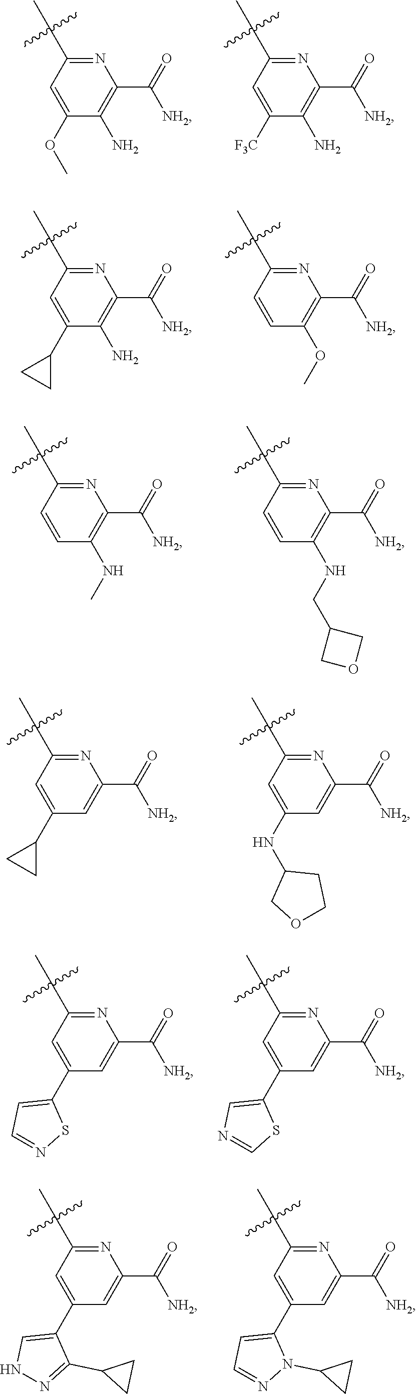

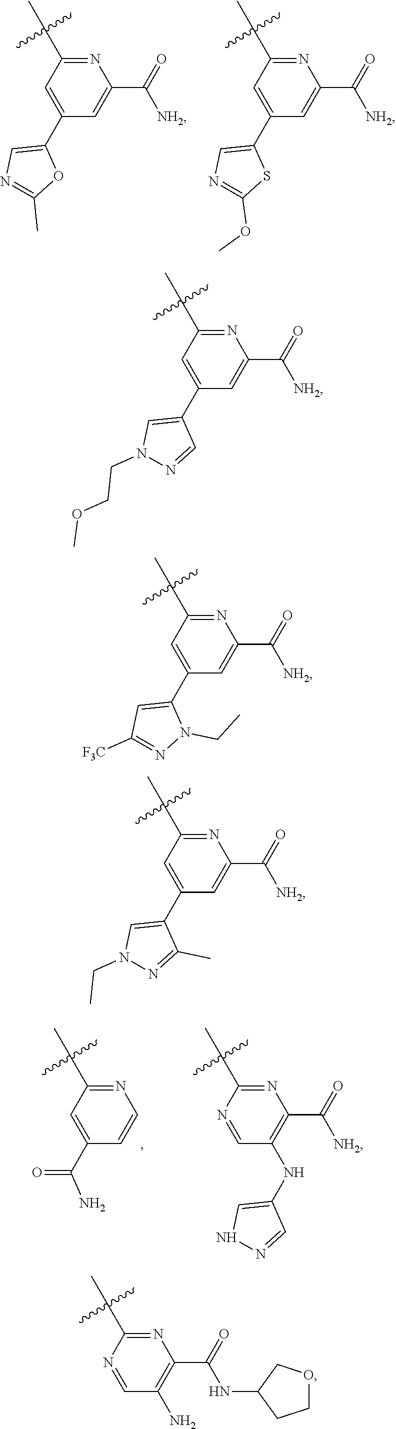

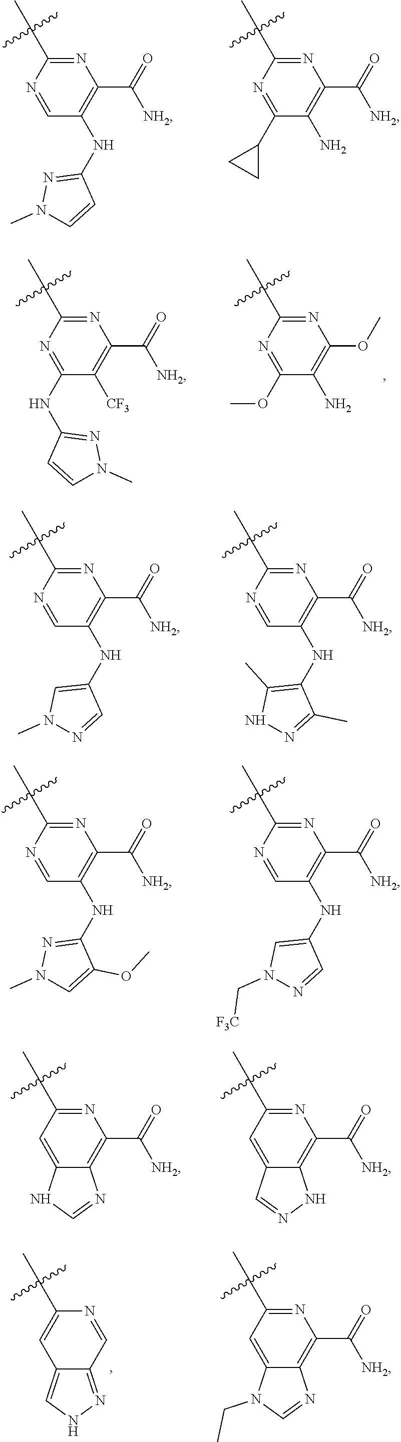

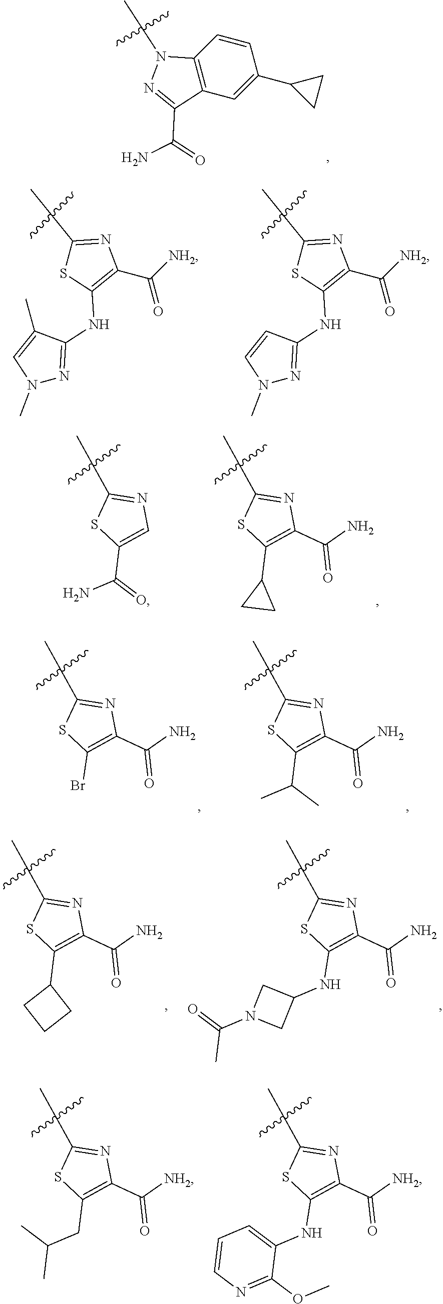

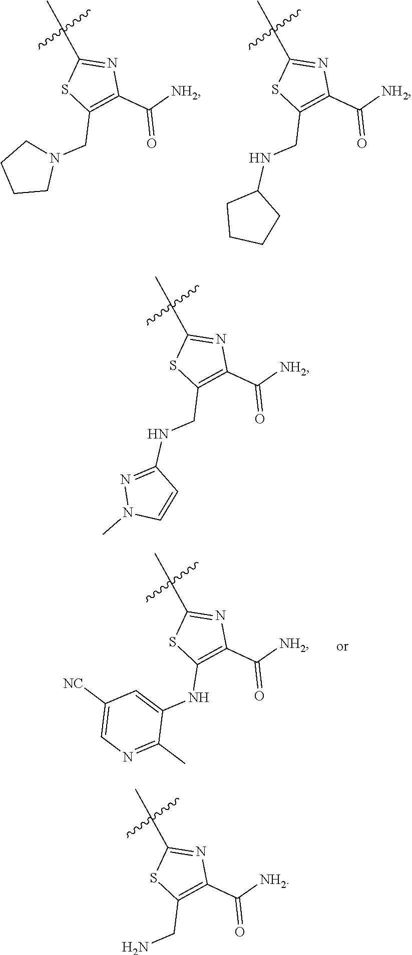 [SMILES: *c1nc(C(N)=O)c(CN2CCCC2)s1 ; *c1nc(C(N)=O)c(CNC2CCCC2)s1 ; *c1nc(C(N)=O)c(CNc2ccn(C)n2)s1 ; *c1nc(C(N)=O)c(Nc2cc(C#N)cnc2C)s1 ; *c1nc(C(N)=O)c(CN)s1]

In some embodiments, disclosed herein is a compound of Formula I, or a pharmaceutically acceptable salt thereof, wherein R² is:

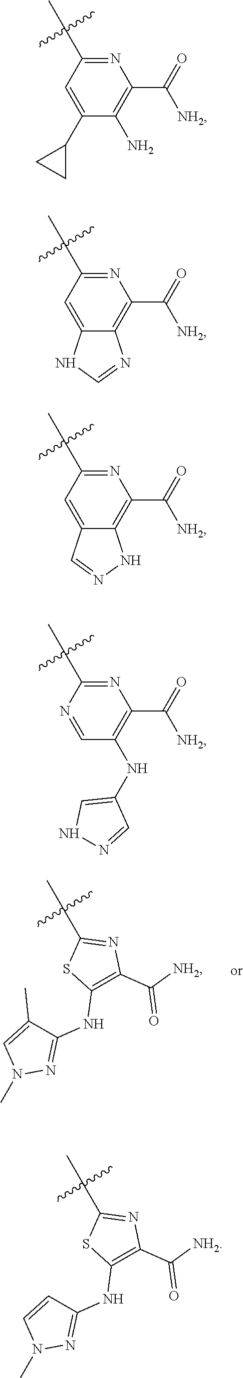 [SMILES: *c1cc(C2CC2)c(N)c(C(N)=O)n1 ; *c1cc2[nH]cnc2c(C(N)=O)n1 ; *c1cc2cn[nH]c2c(C(N)=O)n1 ; *c1ncc(Nc2cn[nH]c2)c(C(N)=O)n1 ; *c1nc(C(N)=O)c(Nc2nn(C)cc2C)s1 ; *c1nc(C(N)=O)c(Nc2ccn(C)n2)s1]

In some embodiments, disclosed herein is a compound of Formula I, or a pharmaceutically acceptable salt thereof, wherein R^{N1} is hydrogen.

In some embodiments, disclosed herein is a compound of Formula I, or a pharmaceutically acceptable salt thereof, wherein R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl. In some embodiments, disclosed herein is a compound of Formula I, or a pharmaceutically acceptable salt thereof, wherein R^{N2} is hydrogen, —C₍₁₋₄₎alkyl, —C₍₃₋₆₎cycloalkyl, 4- to 6-membered heterocyclyl, 5- to 6-membered heteroaryl, wherein the 4- to 6-membered heterocyclyl and 5- to 6-membered heteroaryl are optionally further substituted with one to three groups selected from —C₍₁₋₄₎alkyl and —C₍₁₋₄₎haloalkyl. In some embodiments, disclosed herein is a compound of Formula I, or a pharmaceutically acceptable salt thereof, wherein R^{N2} is hydrogen, 4- to 6-membered heterocyclyl, or 5- to 6-membered heteroaryl, wherein the 5- to 6-membered heteroaryl is optionally further substituted with one to three groups —C₍₁₋₄₎alkyl groups. In some embodiments, disclosed herein is a compound of Formula I, or a pharmaceutically acceptable salt thereof, wherein R^{N2} is hydrogen. In some embodiments, disclosed herein is a compound of Formula I, or a pharmaceutically acceptable salt thereof, wherein R^{N2} is 4- to 6-membered heterocyclyl. In some embodiments, disclosed herein is a compound of Formula I, or a pharmaceutically acceptable salt thereof, wherein R^{N2} is 5- to 6-membered heteroaryl, which is optionally substituted with one to three groups —C₍₁₋₄₎alkyl groups.

In some embodiments, disclosed herein is a compound of Formula I, or a pharmaceutically acceptable salt thereof, wherein R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups.

In some embodiments, disclosed herein is a compound of Formula I, or a pharmaceutically acceptable salt thereof, wherein R^{N4} is hydrogen, —C₍₁₋₄₎alkyl, or 4- to 6-membered heterocyclyl. In some embodiments, disclosed herein is a compound of Formula I, or a pharmaceutically acceptable salt thereof, wherein R^{N4} is hydrogen.

In some embodiments, disclosed herein is a compound of Formula I, or a pharmaceutically acceptable salt thereof, wherein no more than one of V, X, Y, and Z is N. In some embodiments, disclosed herein is a compound of Formula I, or a pharmaceutically acceptable salt thereof, wherein at least three of V, X, Y, and Z are C—H.

In some embodiments, disclosed herein is a compound of Formula I, or a pharmaceutically acceptable salt thereof, wherein if R² is pyrimidinyl, then the pyrimidinyl is substituted with one —C(O)N(R^{N1})(R^{N4}) group and is optionally further substituted with one to two R^{3d} groups.

In some embodiments, disclosed herein is a compound of Formula I, or a pharmaceutically acceptable salt thereof, wherein if R² is pyrimidinyl, then R² has a structure selected from the group consisting of:

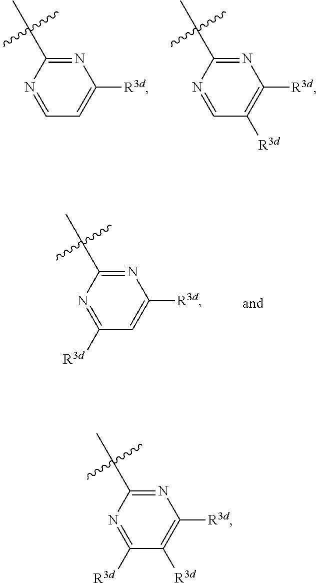

wherein at least one R^{3d} group is —C(O)N(R^{N1})(R^{N4}).

In some embodiments, disclosed herein is a compound of Formula I, or a pharmaceutically acceptable salt thereof, wherein if R² is pyrimidinyl, then R² has a structure selected from the group consisting of:

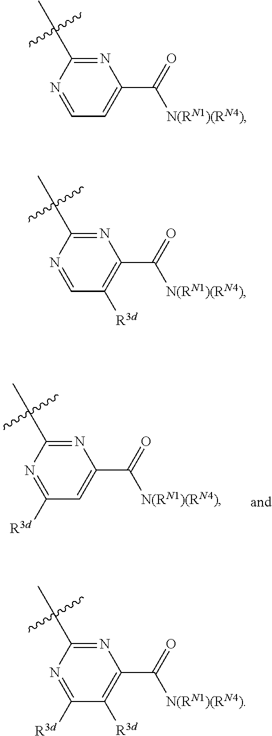

In some embodiments, disclosed herein is a compound of Formula I, or a pharmaceutically acceptable salt thereof, which is a compound of Formula If:

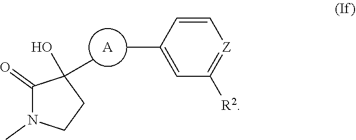

In some embodiments, disclosed herein is a compound of Formula I, or a pharmaceutically acceptable salt thereof, which is a compound of Formula If:

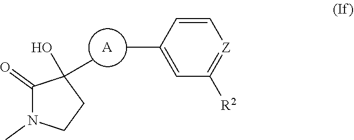

wherein:
A is a isoxazolyl;
Z is N or C—H;
R² is 5- to 10-membered heteroaryl, which is optionally substituted with one to three R^{3d} groups;
each R^{3d} is independently for each occurrence —N(R^{N1})(R^{N2})_C₍₁₋₆₎alkyl, —C₍₁₋₆₎ haloalkyl, —C₍₃₋₁₀₎cycloalkyl, —C(O)N(R^{N1})(R^{N4}), or 5- to 6-membered heteroaryl, wherein the 5- to 6-membered heteroaryl is optionally further substituted with one to three —C₍₁₋₄₎alkyl groups;
R^{N1} is hydrogen;
R^{N2} is hydrogen, 4- to 6-membered heterocyclyl, or 5- to 6-membered heteroaryl, wherein the 5- to 6-membered heteroaryl is optionally further substituted with one to three —C₍₁₋₄₎alkyl groups; and
R^{N4} is hydrogen;
provided that if R² is pyrimidinyl, then the pyrimidinyl is substituted with one —C(O)N(R^{N1})(R^{N4}) group and is optionally further substituted with one to two R^{3d} groups.

In some embodiments, disclosed herein is a compound of Formula I, or a pharmaceutically acceptable salt thereof, having a structure as shown in any one of Tables 1A to 1J.

TABLE 1A

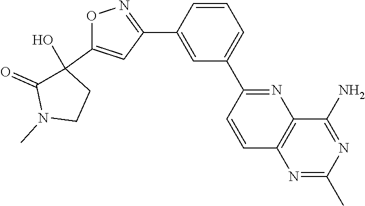 [SMILES: Cc1nc(N)c2nc(-c3cccc(-c4cc(C5(O)CCN(C)C5=O)on4)c3)ccc2n1]

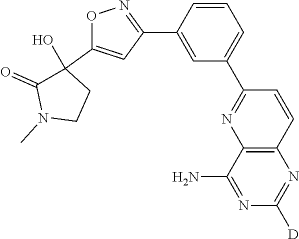 [SMILES: [2H]c1nc(N)c2nc(-c3cccc(-c4cc(C5(O)CCN(C)C5=O)on4)c3)ccc2n1]

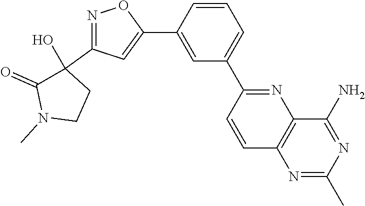 [SMILES: Cc1nc(N)c2nc(-c3cccc(-c4cc(C5(O)CCN(C)C5=O)no4)c3)ccc2n1]

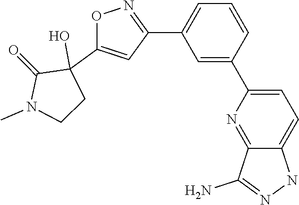 [SMILES: CN1CCC(O)(c2cc(-c3cccc(-c4ccc5[nH]nc(N)c5n4)c3)no2)C1=O]

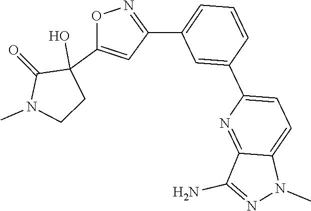

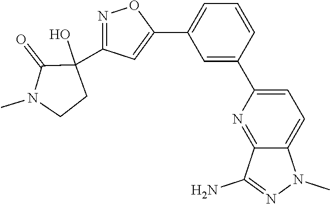

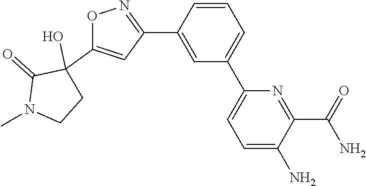 [SMILES: CN1CCC(O)(c2cc(-c3cccc(-c4ccc(N)c(C(N)=O)n4)c3)no2)C1=O]

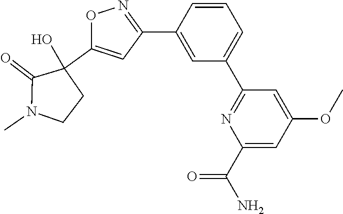

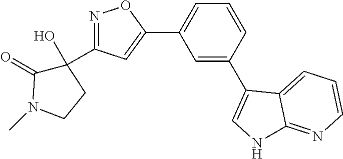 [SMILES: CN1CCC(O)(c2cc(-c3cccc(-c4c[nH]c5ncccc45)c3)on2)C1=O]

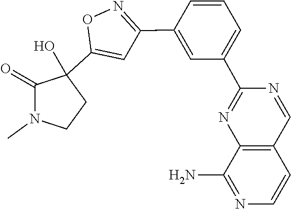 [SMILES: CN1CCC(O)(c2cc(-c3cccc(-c4ncc5ccnc(N)c5n4)c3)no2)C1=O]

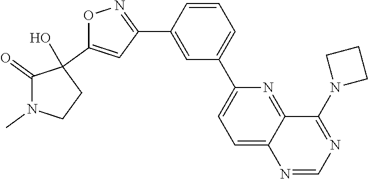 [SMILES: CN1CCC(O)(c2cc(-c3cccc(-c4ccc5ncnc(N6CCC6)c5n4)c3)no2)C1=O]

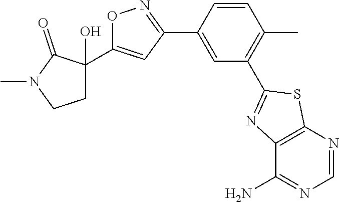 [SMILES: Cc1ccc(-c2cc(C3(O)CCN(C)C3=O)on2)cc1-c1nc2c(N)ncnc2s1]

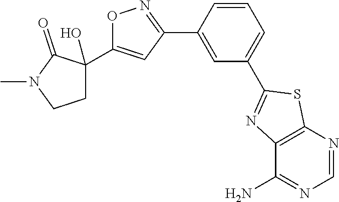 [SMILES: CN1CCC(O)(c2cc(-c3cccc(-c4nc5c(N)ncnc5s4)c3)no2)C1=O]

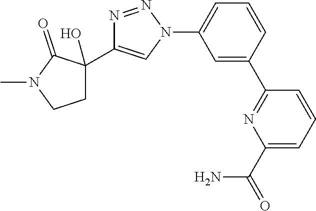 [SMILES: CN1CCC(O)(c2cn(-c3cccc(-c4cccc(C(N)=O)n4)c3)nn2)C1=O]

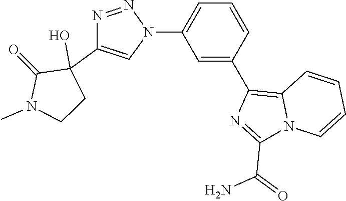 [SMILES: CN1CCC(O)(c2cn(-c3cccc(-c4nc(C(N)=O)n5ccccc45)c3)nn2)C1=O]

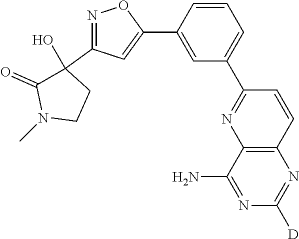 [SMILES: [2H]c1nc(N)c2nc(-c3cccc(-c4cc(C5(O)CCN(C)C5=O)no4)c3)ccc2n1]

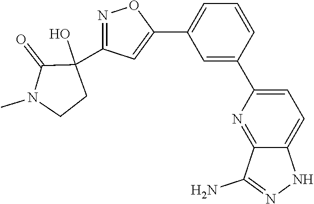 [SMILES: CN1CCC(O)(c2cc(-c3cccc(-c4ccc5[nH]nc(N)c5n4)c3)on2)C1=O]

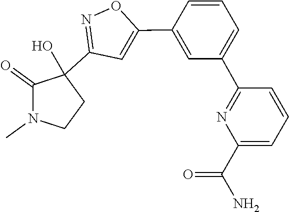 [SMILES: CN1CCC(O)(c2cc(-c3cccc(-c4cccc(C(N)=O)n4)c3)on2)C1=O]

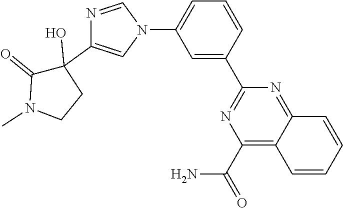 [SMILES: CN1CCC(O)(c2cn(-c3cccc(-c4nc(C(N)=O)c5ccccc5n4)c3)cn2)C1=O]

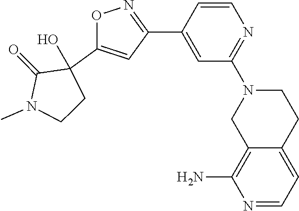 [SMILES: CN1CCC(O)(c2cc(-c3ccnc(N4CCc5ccnc(N)c5C4)c3)no2)C1=O]

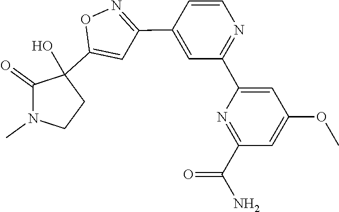 [SMILES: COc1cc(C(N)=O)nc(-c2cc(-c3cc(C4(O)CCN(C)C4=O)on3)ccn2)c1]

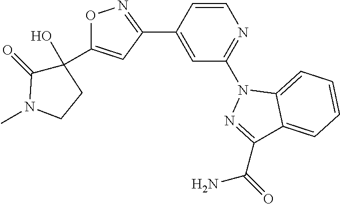 [SMILES: CN1CCC(O)(c2cc(-c3ccnc(-n4nc(C(N)=O)c5ccccc54)c3)no2)C1=O]

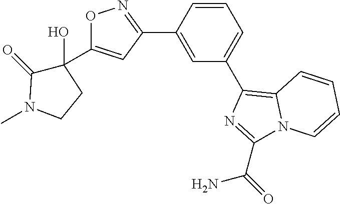 [SMILES: CN1CCC(O)(c2cc(-c3cccc(-c4nc(C(N)=O)n5ccccc45)c3)no2)C1=O]

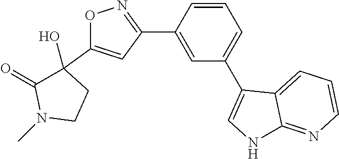 [SMILES: CN1CCC(O)(c2cc(-c3cccc(-c4c[nH]c5ncccc45)c3)no2)C1=O]

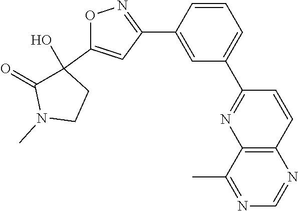 [SMILES: Cc1ncnc2ccc(-c3cccc(-c4cc(C5(O)CCN(C)C5=O)on4)c3)nc12]

TABLE 1B

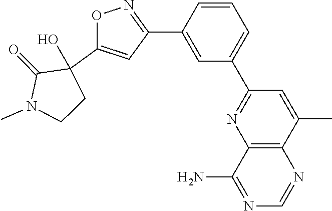

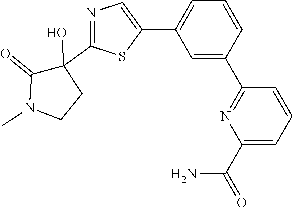 [SMILES: CN1CCC(O)(c2ncc(-c3cccc(-c4cccc(C(N)=O)n4)c3)s2)C1=O]

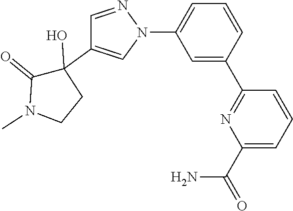 [SMILES: CN1CCC(O)(c2cnn(-c3cccc(-c4cccc(C(N)=O)n4)c3)c2)C1=O]

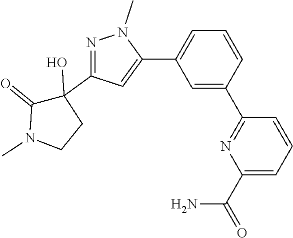 [SMILES: CN1CCC(O)(c2cc(-c3cccc(-c4cccc(C(N)=O)n4)c3)n(C)n2)C1=O]

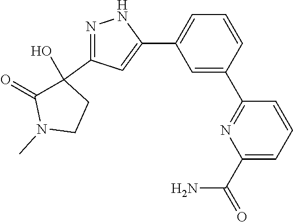 [SMILES: CN1CCC(O)(c2cc(-c3cccc(-c4cccc(C(N)=O)n4)c3)[nH]n2)C1=O]

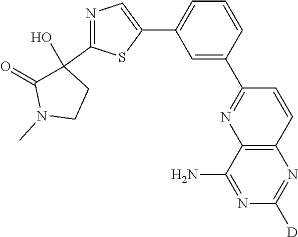 [SMILES: [2H]c1nc(N)c2nc(-c3cccc(-c4cnc(C5(O)CCN(C)C5=O)s4)c3)ccc2n1]

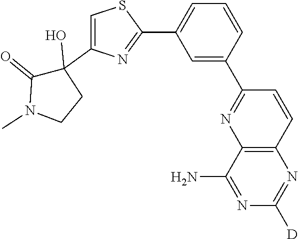 [SMILES: [2H]c1nc(N)c2nc(-c3cccc(-c4nc(C5(O)CCN(C)C5=O)cs4)c3)ccc2n1]

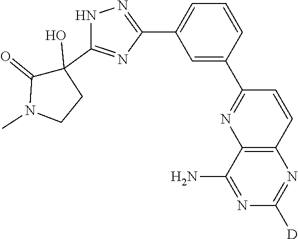 [SMILES: [2H]c1nc(N)c2nc(-c3cccc(-c4n[nH]c(C5(O)CCN(C)C5=O)n4)c3)ccc2n1]

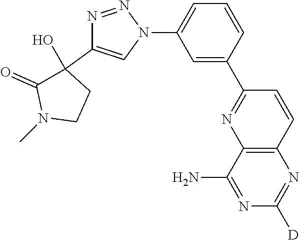 [SMILES: [2H]c1nc(N)c2nc(-c3cccc(-n4cc(C5(O)CCN(C)C5=O)nn4)c3)ccc2n1]

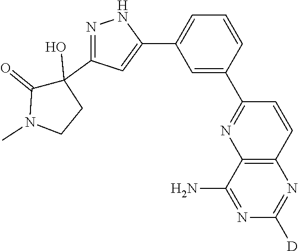 [SMILES: [2H]c1nc(N)c2nc(-c3cccc(-c4cc(C5(O)CCN(C)C5=O)n[nH]4)c3)ccc2n1]

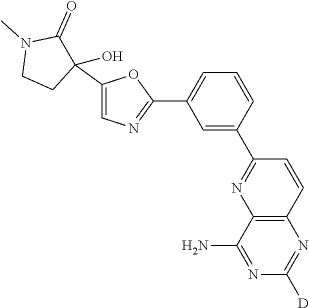 [SMILES: [2H]c1nc(N)c2nc(-c3cccc(-c4ncc(C5(O)CCN(C)C5=O)o4)c3)ccc2n1]

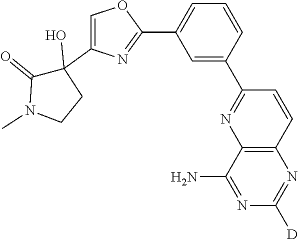 [SMILES: [2H]c1nc(N)c2nc(-c3cccc(-c4nc(C5(O)CCN(C)C5=O)co4)c3)ccc2n1]

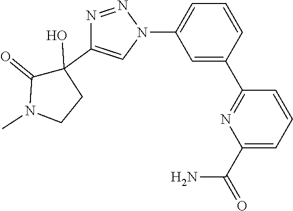 [SMILES: CN1CCC(O)(c2cn(-c3cccc(-c4cccc(C(N)=O)n4)c3)nn2)C1=O]

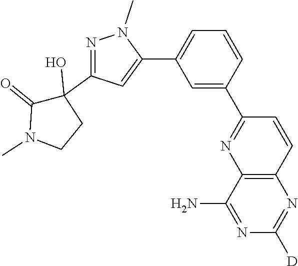 [SMILES: [2H]c1nc(N)c2nc(-c3cccc(-c4cc(C5(O)CCN(C)C5=O)nn4C)c3)ccc2n1]

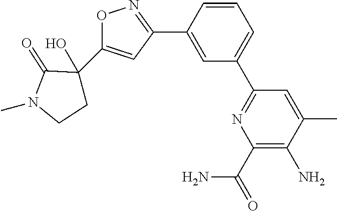

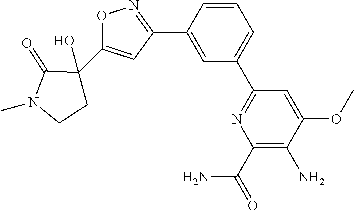

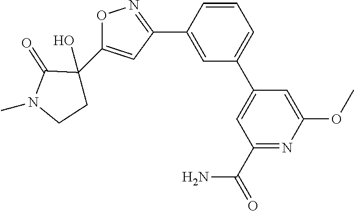

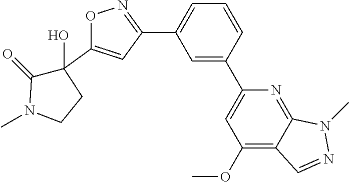

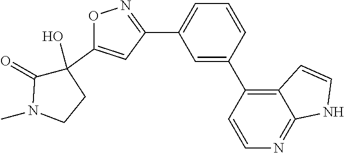 [SMILES: CN1CCC(O)(c2cc(-c3cccc(-c4ccnc5[nH]ccc45)c3)no2)C1=O]

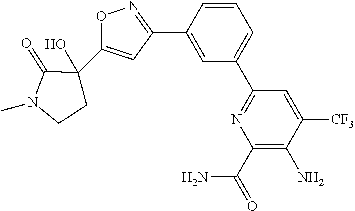 [SMILES: CN1CCC(O)(c2cc(-c3cccc(-c4cc(C(F)(F)F)c(N)c(C(N)=O)n4)c3)no2)C1=O]

TABLE 1C

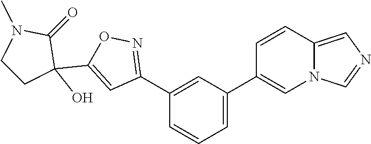 [SMILES: CN1CCC(O)(c2cc(-c3cccc(-c4ccc5cncn5c4)c3)no2)C1=O]

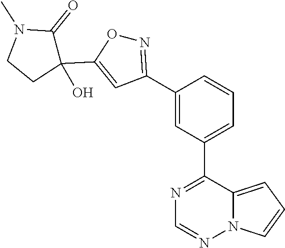 [SMILES: CN1CCC(O)(c2cc(-c3cccc(-c4ncnn5cccc45)c3)no2)C1=O]

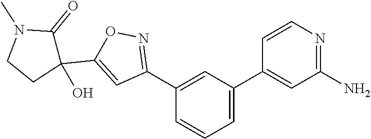 [SMILES: CN1CCC(O)(c2cc(-c3cccc(-c4ccnc(N)c4)c3)no2)C1=O]

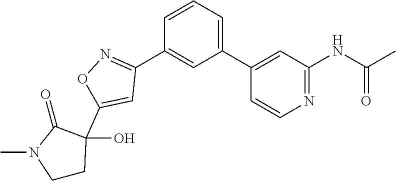 [SMILES: CC(=O)Nc1cc(-c2cccc(-c3cc(C4(O)CCN(C)C4=O)on3)c2)ccn1]

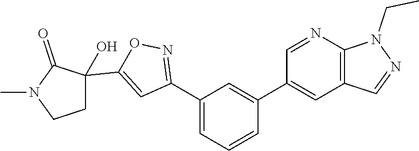 [SMILES: CCn1ncc2cc(-c3cccc(-c4cc(C5(O)CCN(C)C5=O)on4)c3)cnc21]

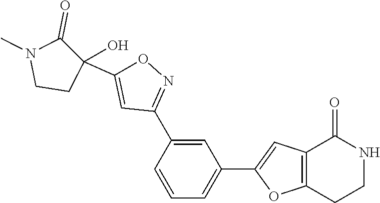 [SMILES: CN1CCC(O)(c2cc(-c3cccc(-c4cc5c(o4)CCNC5=O)c3)no2)C1=O]

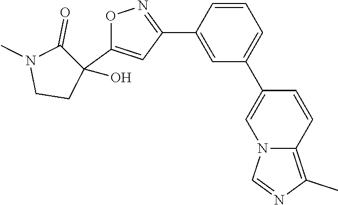

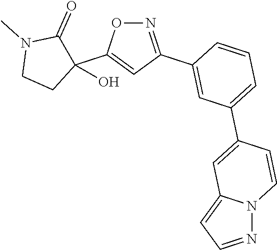 [SMILES: CN1CCC(O)(c2cc(-c3cccc(-c4ccn5nccc5c4)c3)no2)C1=O]

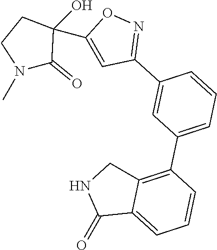 [SMILES: CN1CCC(O)(c2cc(-c3cccc(-c4cccc5c4CNC5=O)c3)no2)C1=O]

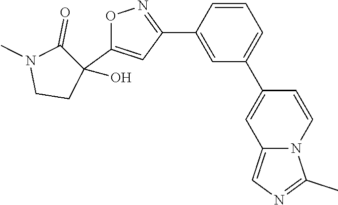

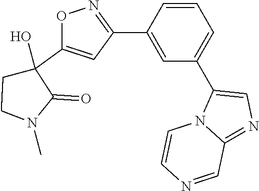 [SMILES: CN1CCC(O)(c2cc(-c3cccc(-c4cnc5cnccn45)c3)no2)C1=O]

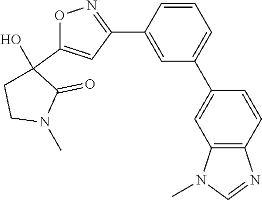 [SMILES: CN1CCC(O)(c2cc(-c3cccc(-c4ccc5ncn(C)c5c4)c3)no2)C1=O]

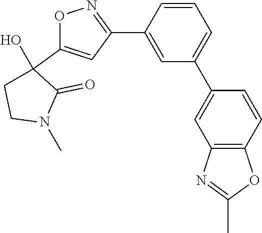 [SMILES: Cc1nc2cc(-c3cccc(-c4cc(C5(O)CCN(C)C5=O)on4)c3)ccc2o1]

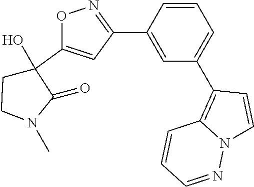 [SMILES: CN1CCC(O)(c2cc(-c3cccc(-c4ccn5ncccc45)c3)no2)C1=O]

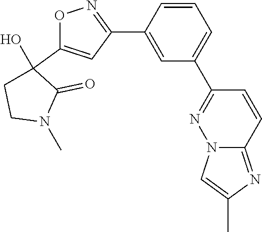 [SMILES: Cc1cn2nc(-c3cccc(-c4cc(C5(O)CCN(C)C5=O)on4)c3)ccc2n1]

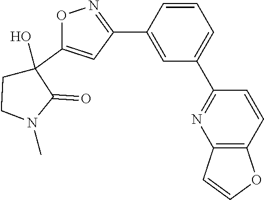 [SMILES: CN1CCC(O)(c2cc(-c3cccc(-c4ccc5occc5n4)c3)no2)C1=O]

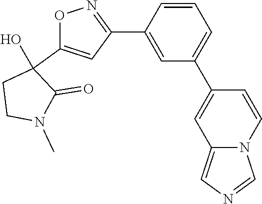 [SMILES: CN1CCC(O)(c2cc(-c3cccc(-c4ccn5cncc5c4)c3)no2)C1=O]

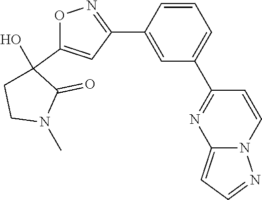 [SMILES: CN1CCC(O)(c2cc(-c3cccc(-c4ccn5nccc5n4)c3)no2)C1=O]

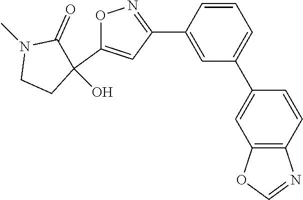 [SMILES: CN1CCC(O)(c2cc(-c3cccc(-c4ccc5ncoc5c4)c3)no2)C1=O]

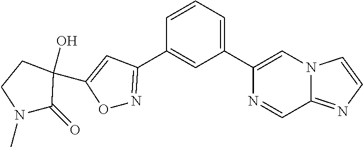 [SMILES: CN1CCC(O)(c2cc(-c3cccc(-c4cn5ccnc5cn4)c3)no2)C1=O]

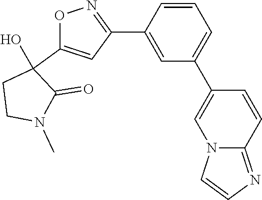 [SMILES: CN1CCC(O)(c2cc(-c3cccc(-c4ccc5nccn5c4)c3)no2)C1=O]

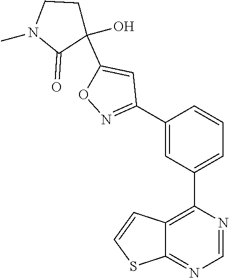 [SMILES: CN1CCC(O)(c2cc(-c3cccc(-c4ncnc5sccc45)c3)no2)C1=O]

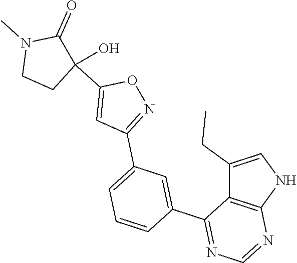 [SMILES: CCc1c[nH]c2ncnc(-c3cccc(-c4cc(C5(O)CCN(C)C5=O)on4)c3)c12]

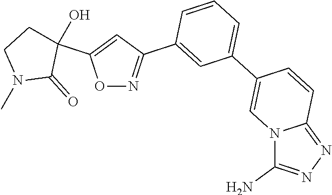 [SMILES: CN1CCC(O)(c2cc(-c3cccc(-c4ccc5nnc(N)n5c4)c3)no2)C1=O]

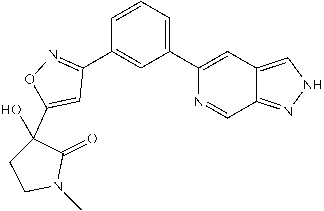 [SMILES: CN1CCC(O)(c2cc(-c3cccc(-c4cc5c[nH]nc5cn4)c3)no2)C1=O]

TABLE 1D

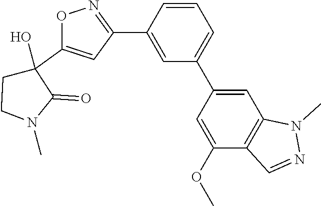 [SMILES: COc1cc(-c2cccc(-c3cc(C4(O)CCN(C)C4=O)on3)c2)cc2c1cnn2C]

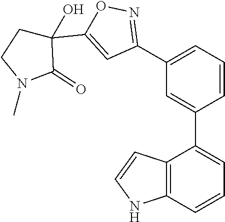 [SMILES: CN1CCC(O)(c2cc(-c3cccc(-c4cccc5[nH]ccc45)c3)no2)C1=O]

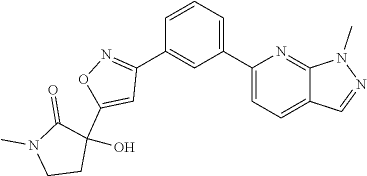 [SMILES: CN1CCC(O)(c2cc(-c3cccc(-c4ccc5cnn(C)c5n4)c3)no2)C1=O]

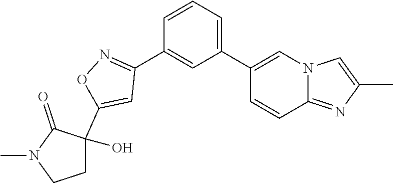

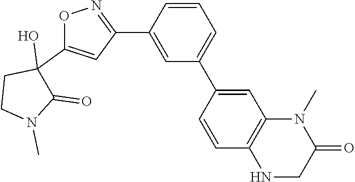 [SMILES: CN1CCC(O)(c2cc(-c3cccc(-c4ccc5c(c4)N(C)C(=O)CN5)c3)no2)C1=O]

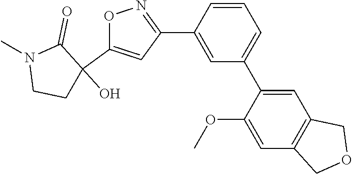 [SMILES: COc1cc2c(cc1-c1cccc(-c3cc(C4(O)CCN(C)C4=O)on3)c1)COC2]

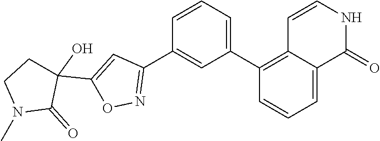 [SMILES: CN1CCC(O)(c2cc(-c3cccc(-c4cccc5c(=O)[nH]ccc45)c3)no2)C1=O]

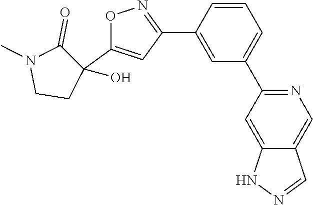 [SMILES: CN1CCC(O)(c2cc(-c3cccc(-c4cc5[nH]ncc5cn4)c3)no2)C1=O]

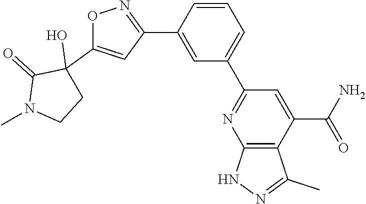 [SMILES: Cc1n[nH]c2nc(-c3cccc(-c4cc(C5(O)CCN(C)C5=O)on4)c3)cc(C(N)=O)c12]

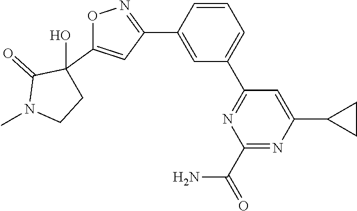 [SMILES: CN1CCC(O)(c2cc(-c3cccc(-c4cc(C5CC5)nc(C(N)=O)n4)c3)no2)C1=O]

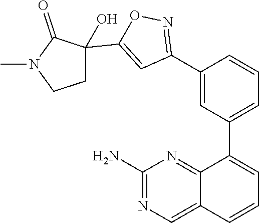 [SMILES: CN1CCC(O)(c2cc(-c3cccc(-c4cccc5cnc(N)nc45)c3)no2)C1=O]

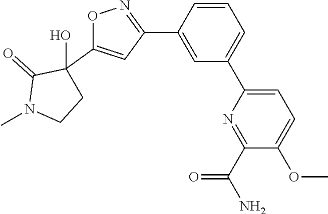

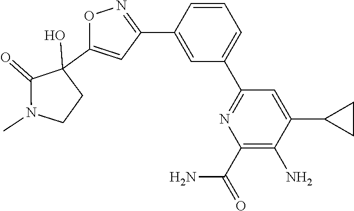 [SMILES: CN1CCC(O)(c2cc(-c3cccc(-c4cc(C5CC5)c(N)c(C(N)=O)n4)c3)no2)C1=O]

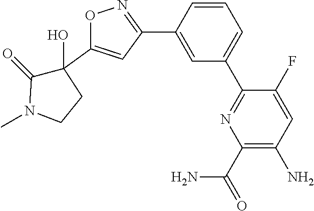 [SMILES: CN1CCC(O)(c2cc(-c3cccc(-c4nc(C(N)=O)c(N)cc4F)c3)no2)C1=O]

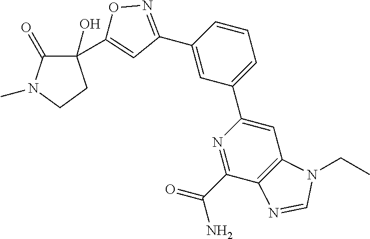 [SMILES: CCn1cnc2c(C(N)=O)nc(-c3cccc(-c4cc(C5(O)CCN(C)C5=O)on4)c3)cc21]

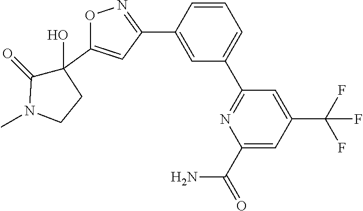 [SMILES: CN1CCC(O)(c2cc(-c3cccc(-c4cc(C(F)(F)F)cc(C(N)=O)n4)c3)no2)C1=O]

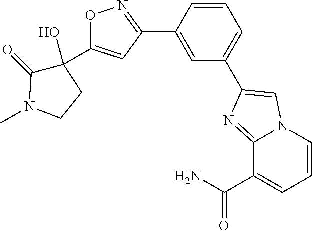 [SMILES: CN1CCC(O)(c2cc(-c3cccc(-c4cn5cccc(C(N)=O)c5n4)c3)no2)C1=O]

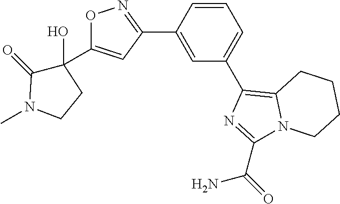 [SMILES: CN1CCC(O)(c2cc(-c3cccc(-c4nc(C(N)=O)n5c4CCCC5)c3)no2)C1=O]

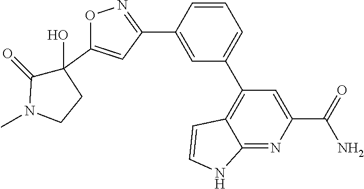 [SMILES: CN1CCC(O)(c2cc(-c3cccc(-c4cc(C(N)=O)nc5[nH]ccc45)c3)no2)C1=O]

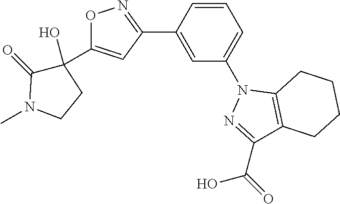 [SMILES: CN1CCC(O)(c2cc(-c3cccc(-n4nc(C(=O)O)c5c4CCCC5)c3)no2)C1=O]

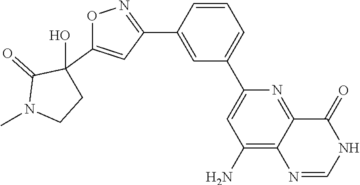 [SMILES: CN1CCC(O)(c2cc(-c3cccc(-c4cc(N)c5nc[nH]c(=O)c5n4)c3)no2)C1=O]

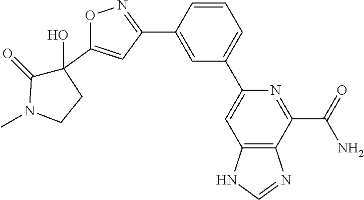 [SMILES: CN1CCC(O)(c2cc(-c3cccc(-c4cc5[nH]cnc5c(C(N)=O)n4)c3)no2)C1=O]

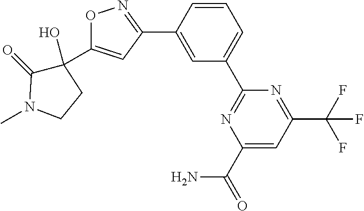 [SMILES: CN1CCC(O)(c2cc(-c3cccc(-c4nc(C(N)=O)cc(C(F)(F)F)n4)c3)no2)C1=O]

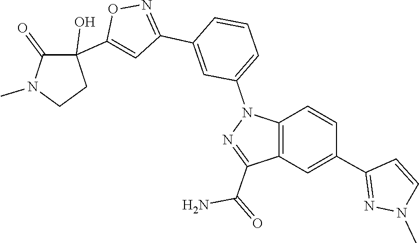 [SMILES: CN1CCC(O)(c2cc(-c3cccc(-n4nc(C(N)=O)c5cc(-c6ccn(C)n6)ccc54)c3)no2)C1=O]

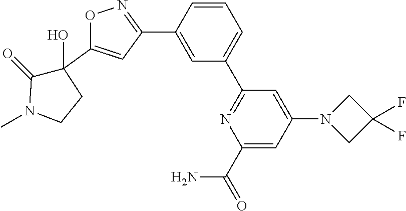 [SMILES: CN1CCC(O)(c2cc(-c3cccc(-c4cc(N5CC(F)(F)C5)cc(C(N)=O)n4)c3)no2)C1=O]

TABLE 1E

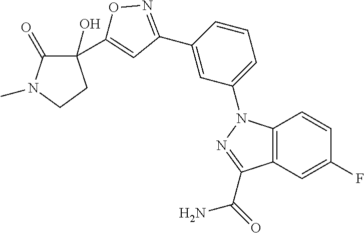 [SMILES: CN1CCC(O)(c2cc(-c3cccc(-n4nc(C(N)=O)c5cc(F)ccc54)c3)no2)C1=O]

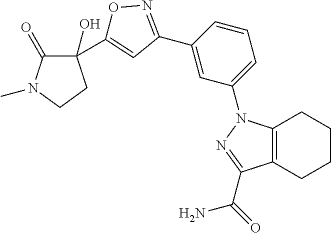 [SMILES: CN1CCC(O)(c2cc(-c3cccc(-n4nc(C(N)=O)c5c4CCCC5)c3)no2)C1=O]

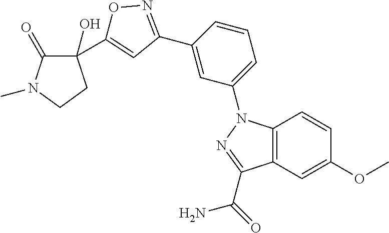

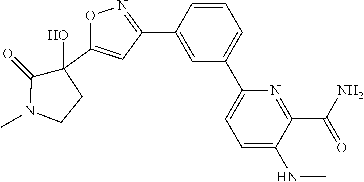 [SMILES: CNc1ccc(-c2cccc(-c3cc(C4(O)CCN(C)C4=O)on3)c2)nc1C(N)=O]

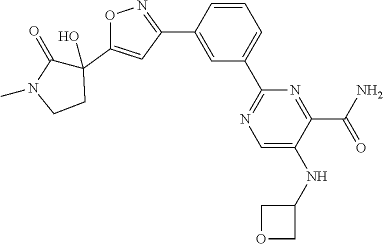 [SMILES: CN1CCC(O)(c2cc(-c3cccc(-c4ncc(NC5COC5)c(C(N)=O)n4)c3)no2)C1=O]

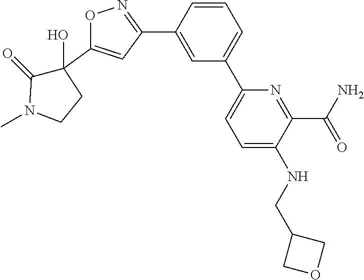 [SMILES: CN1CCC(O)(c2cc(-c3cccc(-c4ccc(NCC5COC5)c(C(N)=O)n4)c3)no2)C1=O]

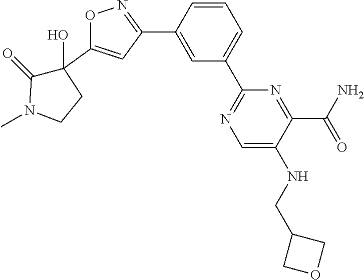 [SMILES: CN1CCC(O)(c2cc(-c3cccc(-c4ncc(NCC5COC5)c(C(N)=O)n4)c3)no2)C1=O]

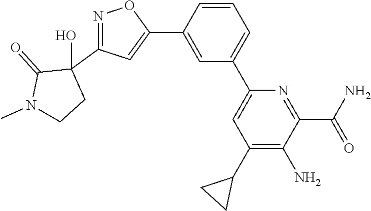 [SMILES: CN1CCC(O)(c2cc(-c3cccc(-c4cc(C5CC5)c(N)c(C(N)=O)n4)c3)on2)C1=O]

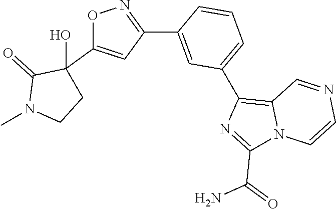 [SMILES: CN1CCC(O)(c2cc(-c3cccc(-c4nc(C(N)=O)n5ccncc45)c3)no2)C1=O]

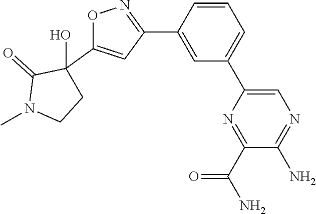 [SMILES: CN1CCC(O)(c2cc(-c3cccc(-c4cnc(N)c(C(N)=O)n4)c3)no2)C1=O]

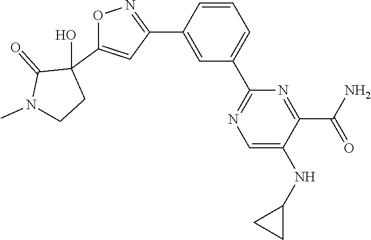 [SMILES: CN1CCC(O)(c2cc(-c3cccc(-c4ncc(NC5CC5)c(C(N)=O)n4)c3)no2)C1=O]

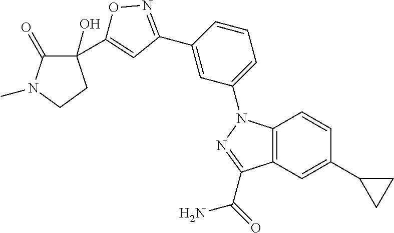 [SMILES: CN1CCC(O)(c2cc(-c3cccc(-n4nc(C(N)=O)c5cc(C6CC6)ccc54)c3)no2)C1=O]

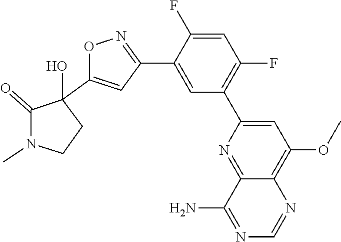

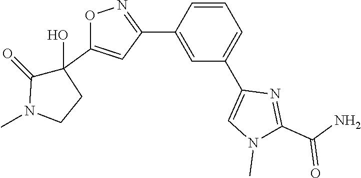 [SMILES: CN1CCC(O)(c2cc(-c3cccc(-c4cn(C)c(C(N)=O)n4)c3)no2)C1=O]

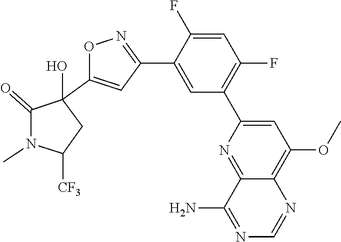

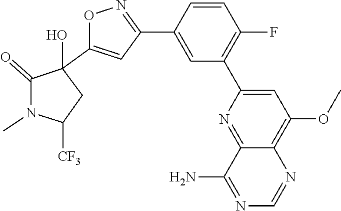

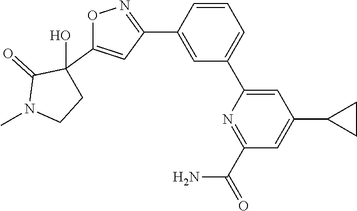 [SMILES: CN1CCC(O)(c2cc(-c3cccc(-c4cc(C5CC5)cc(C(N)=O)n4)c3)no2)C1=O]

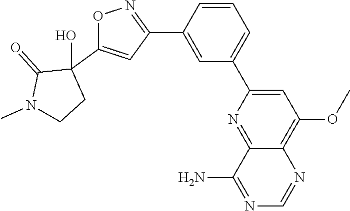

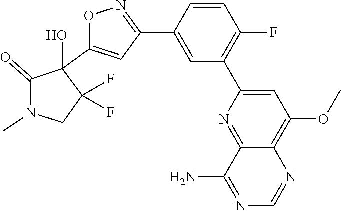

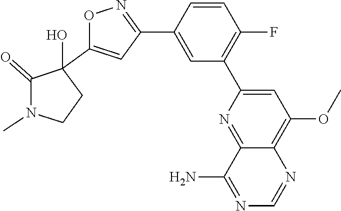

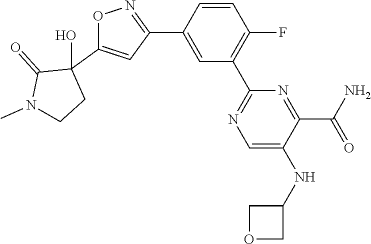 [SMILES: CN1CCC(O)(c2cc(-c3ccc(F)c(-c4ncc(NC5COC5)c(C(N)=O)n4)c3)no2)C1=O]

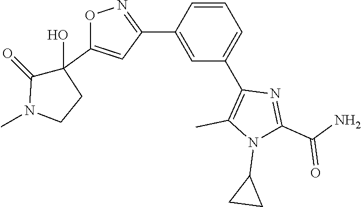 [SMILES: Cc1c(-c2cccc(-c3cc(C4(O)CCN(C)C4=O)on3)c2)nc(C(N)=O)n1C1CC1]

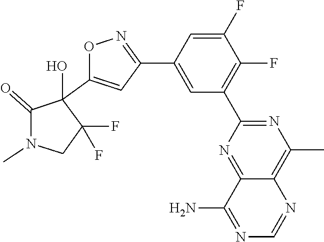

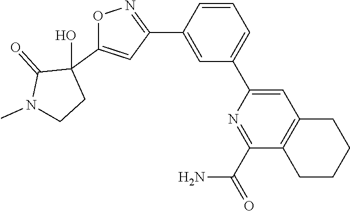 [SMILES: CN1CCC(O)(c2cc(-c3cccc(-c4cc5c(c(C(N)=O)n4)CCCC5)c3)no2)C1=O]

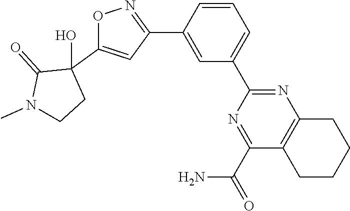 [SMILES: CN1CCC(O)(c2cc(-c3cccc(-c4nc5c(c(C(N)=O)n4)CCCC5)c3)no2)C1=O]

TABLE 1F

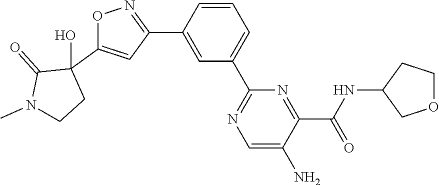 [SMILES: CN1CCC(O)(c2cc(-c3cccc(-c4ncc(N)c(C(=O)NC5CCOC5)n4)c3)no2)C1=O]

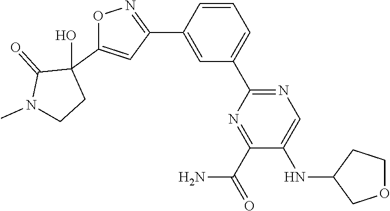 [SMILES: CN1CCC(O)(c2cc(-c3cccc(-c4ncc(NC5CCOC5)c(C(N)=O)n4)c3)no2)C1=O]

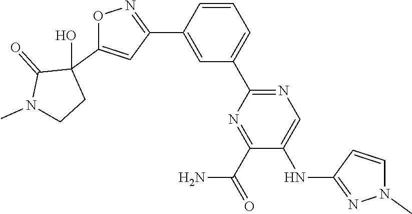

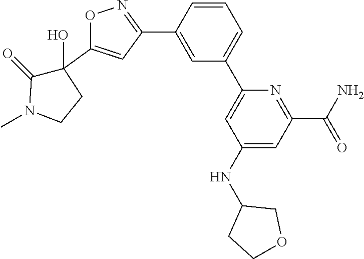 [SMILES: CN1CCC(O)(c2cc(-c3cccc(-c4cc(NC5CCOC5)cc(C(N)=O)n4)c3)no2)C1=O]

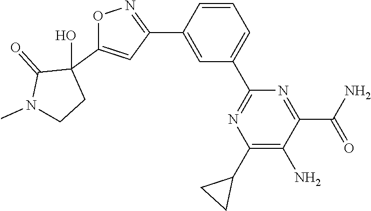 [SMILES: CN1CCC(O)(c2cc(-c3cccc(-c4nc(C(N)=O)c(N)c(C5CC5)n4)c3)no2)C1=O]

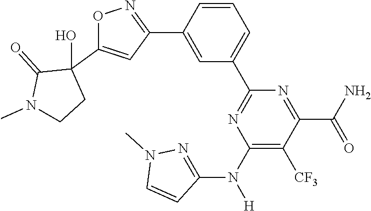 [SMILES: CN1CCC(O)(c2cc(-c3cccc(-c4nc(Nc5ccn(C)n5)c(C(F)(F)F)c(C(N)=O)n4)c3)no2)C1=O]

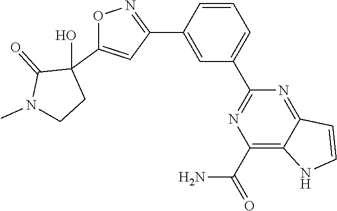 [SMILES: CN1CCC(O)(c2cc(-c3cccc(-c4nc(C(N)=O)c5[nH]ccc5n4)c3)no2)C1=O]

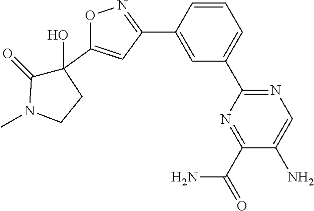 [SMILES: CN1CCC(O)(c2cc(-c3cccc(-c4ncc(N)c(C(N)=O)n4)c3)no2)C1=O]

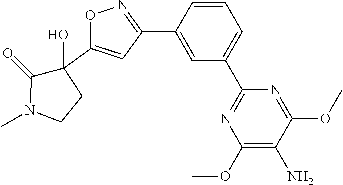

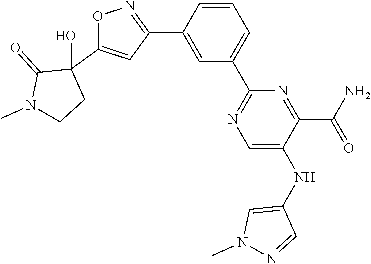 [SMILES: CN1CCC(O)(c2cc(-c3cccc(-c4ncc(Nc5cnn(C)c5)c(C(N)=O)n4)c3)no2)C1=O]

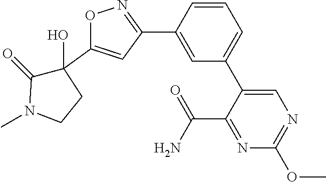

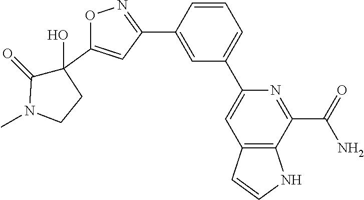 [SMILES: CN1CCC(O)(c2cc(-c3cccc(-c4cc5cc[nH]c5c(C(N)=O)n4)c3)no2)C1=O]

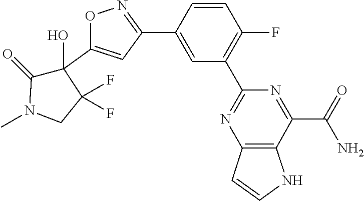 [SMILES: CN1CC(F)(F)C(O)(c2cc(-c3ccc(F)c(-c4nc(C(N)=O)c5[nH]ccc5n4)c3)no2)C1=O]

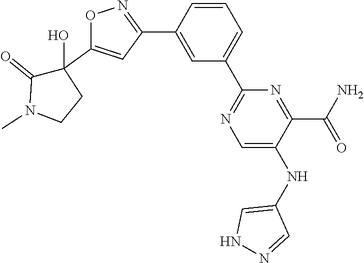 [SMILES: CN1CCC(O)(c2cc(-c3cccc(-c4ncc(Nc5cn[nH]c5)c(C(N)=O)n4)c3)no2)C1=O]

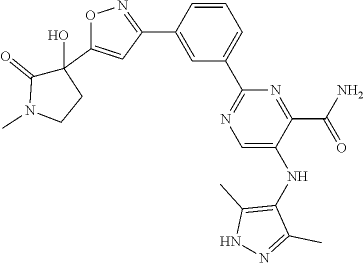 [SMILES: Cc1n[nH]c(C)c1Nc1cnc(-c2cccc(-c3cc(C4(O)CCN(C)C4=O)on3)c2)nc1C(N)=O]

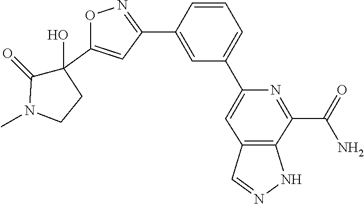 [SMILES: CN1CCC(O)(c2cc(-c3cccc(-c4cc5cn[nH]c5c(C(N)=O)n4)c3)no2)C1=O]

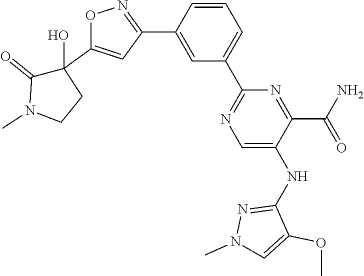 [SMILES: COc1cn(C)nc1Nc1cnc(-c2cccc(-c3cc(C4(O)CCN(C)C4=O)on3)c2)nc1C(N)=O]

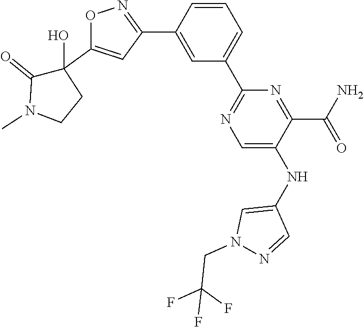 [SMILES: CN1CCC(O)(c2cc(-c3cccc(-c4ncc(Nc5cnn(CC(F)(F)F)c5)c(C(N)=O)n4)c3)no2)C1=O]

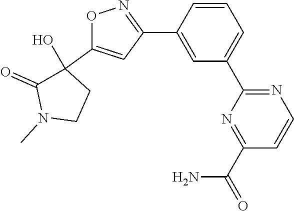 [SMILES: CN1CCC(O)(c2cc(-c3cccc(-c4nccc(C(N)=O)n4)c3)no2)C1=O]

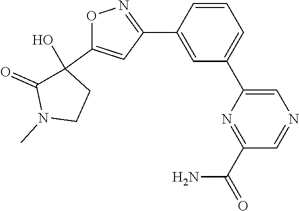 [SMILES: CN1CCC(O)(c2cc(-c3cccc(-c4cncc(C(N)=O)n4)c3)no2)C1=O]

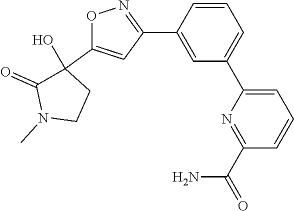 [SMILES: CN1CCC(O)(c2cc(-c3cccc(-c4cccc(C(N)=O)n4)c3)no2)C1=O]

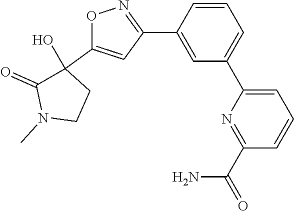 [SMILES: CN1CCC(O)(c2cc(-c3cccc(-c4cccc(C(N)=O)n4)c3)no2)C1=O]

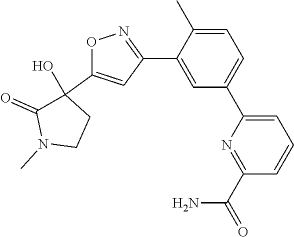 [SMILES: Cc1ccc(-c2cccc(C(N)=O)n2)cc1-c1cc(C2(O)CCN(C)C2=O)on1]

TABLE 1G

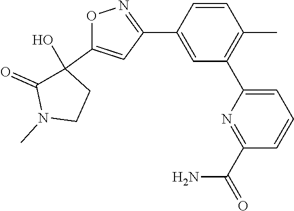 [SMILES: Cc1ccc(-c2cc(C3(O)CCN(C)C3=O)on2)cc1-c1cccc(C(N)=O)n1]

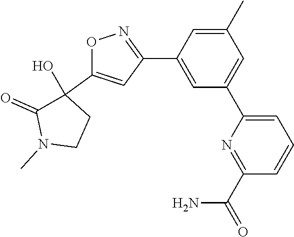 [SMILES: Cc1cc(-c2cc(C3(O)CCN(C)C3=O)on2)cc(-c2cccc(C(N)=O)n2)c1]

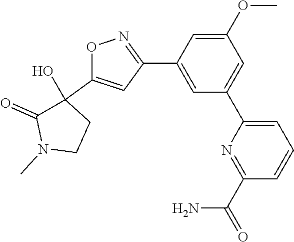 [SMILES: COc1cc(-c2cc(C3(O)CCN(C)C3=O)on2)cc(-c2cccc(C(N)=O)n2)c1]

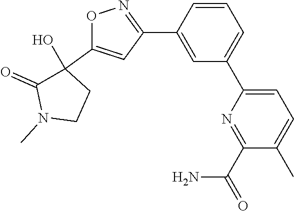 [SMILES: Cc1ccc(-c2cccc(-c3cc(C4(O)CCN(C)C4=O)on3)c2)nc1C(N)=O]

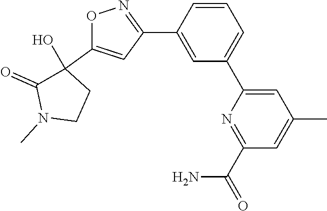 [SMILES: Cc1cc(C(N)=O)nc(-c2cccc(-c3cc(C4(O)CCN(C)C4=O)on3)c2)c1]

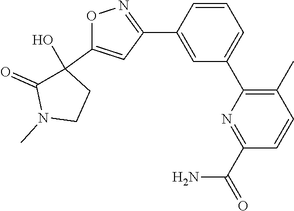 [SMILES: Cc1ccc(C(N)=O)nc1-c1cccc(-c2cc(C3(O)CCN(C)C3=O)on2)c1]

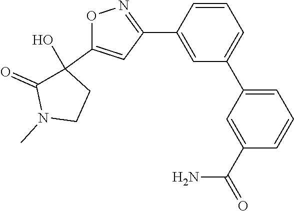 [SMILES: CN1CCC(O)(c2cc(-c3cccc(-c4cccc(C(N)=O)c4)c3)no2)C1=O]

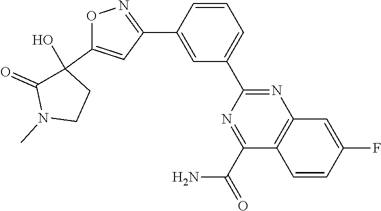 [SMILES: CN1CCC(O)(c2cc(-c3cccc(-c4nc(C(N)=O)c5ccc(F)cc5n4)c3)no2)C1=O]

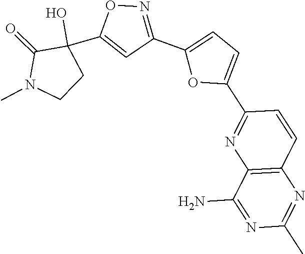

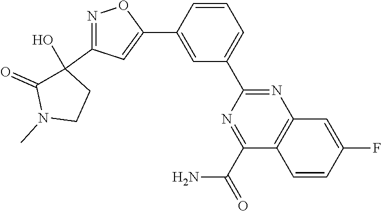 [SMILES: CN1CCC(O)(c2cc(-c3cccc(-c4nc(C(N)=O)c5ccc(F)cc5n4)c3)on2)C1=O]

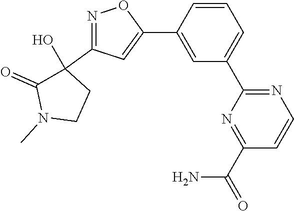 [SMILES: CN1CCC(O)(c2cc(-c3cccc(-c4nccc(C(N)=O)n4)c3)on2)C1=O]

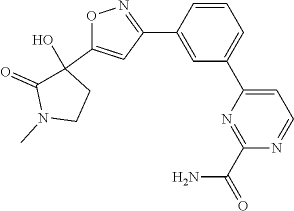 [SMILES: CN1CCC(O)(c2cc(-c3cccc(-c4ccnc(C(N)=O)n4)c3)no2)C1=O]

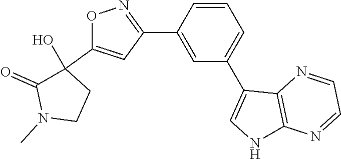 [SMILES: CN1CCC(O)(c2cc(-c3cccc(-c4c[nH]c5nccnc45)c3)no2)C1=O]

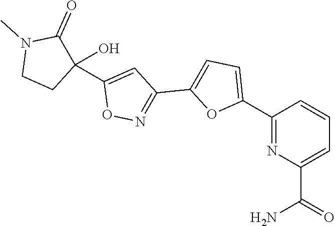 [SMILES: CN1CCC(O)(c2cc(-c3ccc(-c4cccc(C(N)=O)n4)o3)no2)C1=O]

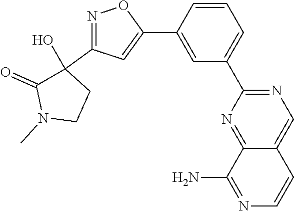 [SMILES: CN1CCC(O)(c2cc(-c3cccc(-c4ncc5ccnc(N)c5n4)c3)on2)C1=O]

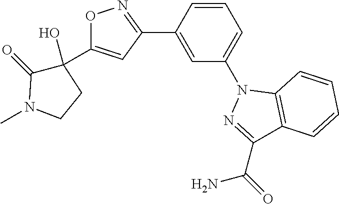 [SMILES: CN1CCC(O)(c2cc(-c3cccc(-n4nc(C(N)=O)c5ccccc54)c3)no2)C1=O]

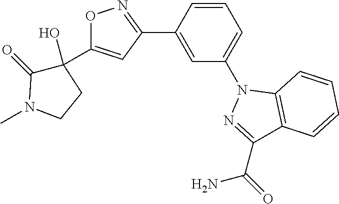 [SMILES: CN1CCC(O)(c2cc(-c3cccc(-n4nc(C(N)=O)c5ccccc54)c3)no2)C1=O]

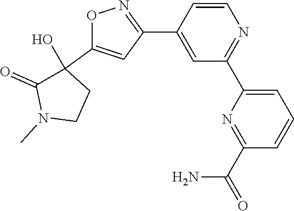 [SMILES: CN1CCC(O)(c2cc(-c3ccnc(-c4cccc(C(N)=O)n4)c3)no2)C1=O]

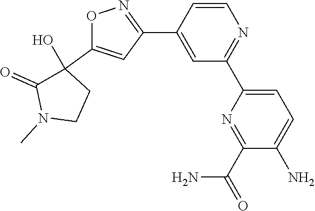 [SMILES: CN1CCC(O)(c2cc(-c3ccnc(-c4ccc(N)c(C(N)=O)n4)c3)no2)C1=O]

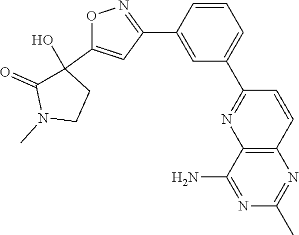 [SMILES: Cc1nc(N)c2nc(-c3cccc(-c4cc(C5(O)CCN(C)C5=O)on4)c3)ccc2n1]

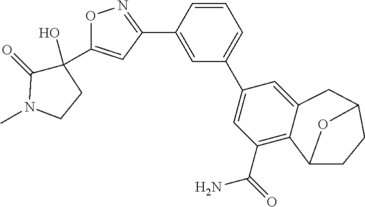 [SMILES: CN1CCC(O)(c2cc(-c3cccc(-c4cc5c(c(C(N)=O)c4)C4CCC(C5)O4)c3)no2)C1=O]

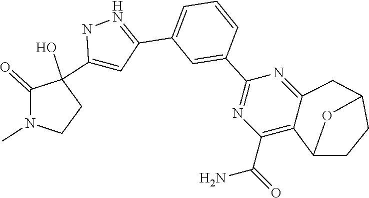

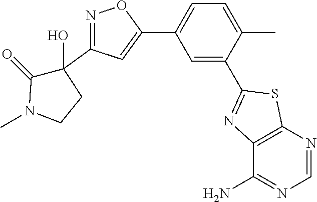 [SMILES: Cc1ccc(-c2cc(C3(O)CCN(C)C3=O)no2)cc1-c1nc2c(N)ncnc2s1]

TABLE 1H

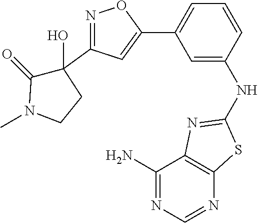 [SMILES: CN1CCC(O)(c2cc(-c3cccc(Nc4nc5c(N)ncnc5s4)c3)on2)C1=O]

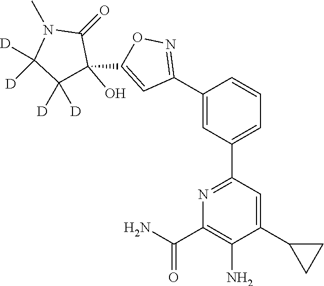 [SMILES: [2H]C1([2H])N(C)C(=O)[C@](O)(c2cc(-c3cccc(-c4cc(C5CC5)c(N)c(C(N)=O)n4)c3)no2)C1([2H])[2H]]

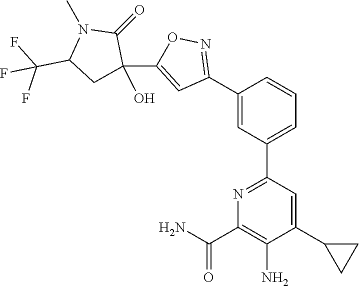 [SMILES: CN1C(=O)C(O)(c2cc(-c3cccc(-c4cc(C5CC5)c(N)c(C(N)=O)n4)c3)no2)CC1C(F)(F)F]

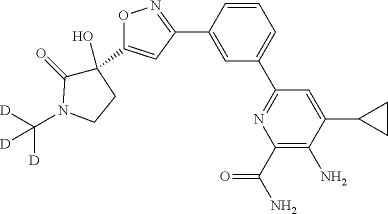 [SMILES: [2H]C([2H])([2H])N1CC[C@@](O)(c2cc(-c3cccc(-c4cc(C5CC5)c(N)c(C(N)=O)n4)c3)no2)C1=O]

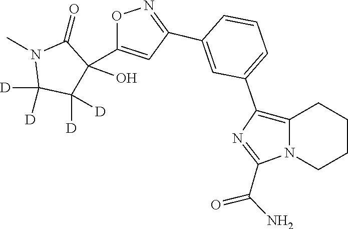 [SMILES: [2H]C1([2H])N(C)C(=O)C(O)(c2cc(-c3cccc(-c4nc(C(N)=O)n5c4CCCC5)c3)no2)C1([2H])[2H]]

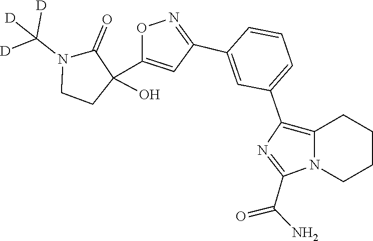 [SMILES: [2H]C([2H])([2H])N1CCC(O)(c2cc(-c3cccc(-c4nc(C(N)=O)n5c4CCCC5)c3)no2)C1=O]

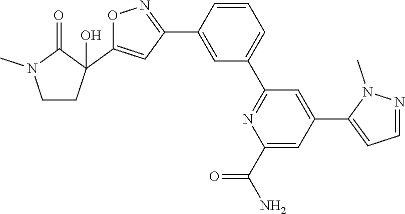 [SMILES: CN1CCC(O)(c2cc(-c3cccc(-c4cc(-c5ccnn5C)cc(C(N)=O)n4)c3)no2)C1=O]

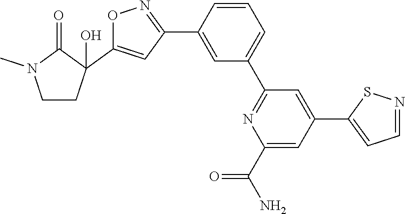 [SMILES: CN1CCC(O)(c2cc(-c3cccc(-c4cc(-c5ccns5)cc(C(N)=O)n4)c3)no2)C1=O]

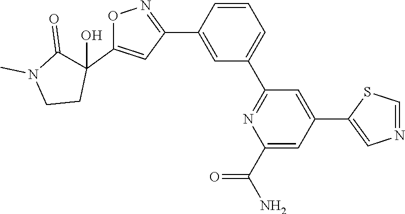 [SMILES: CN1CCC(O)(c2cc(-c3cccc(-c4cc(-c5cncs5)cc(C(N)=O)n4)c3)no2)C1=O]

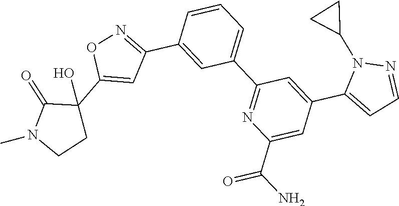 [SMILES: CN1CCC(O)(c2cc(-c3cccc(-c4cc(-c5ccnn5C5CC5)cc(C(N)=O)n4)c3)no2)C1=O]

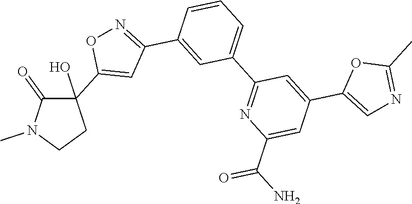

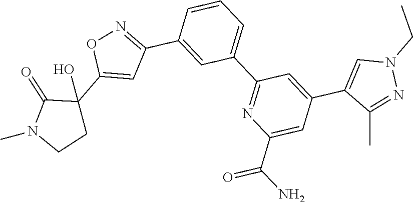 [SMILES: CCn1cc(-c2cc(C(N)=O)nc(-c3cccc(-c4cc(C5(O)CCN(C)C5=O)on4)c3)c2)c(C)n1]

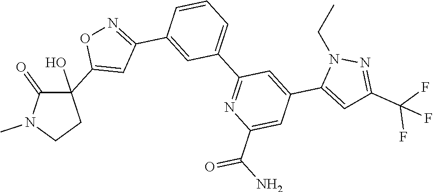 [SMILES: CCn1nc(C(F)(F)F)cc1-c1cc(C(N)=O)nc(-c2cccc(-c3cc(C4(O)CCN(C)C4=O)on3)c2)c1]

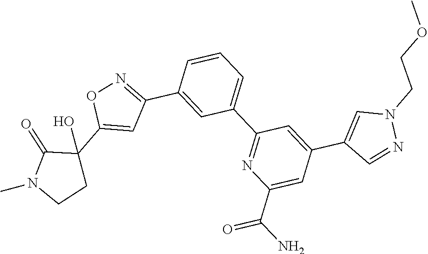 [SMILES: COCCn1cc(-c2cc(C(N)=O)nc(-c3cccc(-c4cc(C5(O)CCN(C)C5=O)on4)c3)c2)cn1]

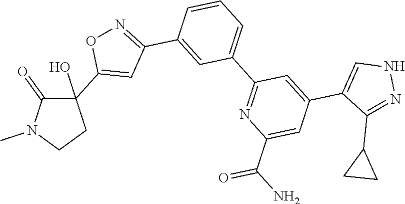 [SMILES: CN1CCC(O)(c2cc(-c3cccc(-c4cc(-c5c[nH]nc5C5CC5)cc(C(N)=O)n4)c3)no2)C1=O]

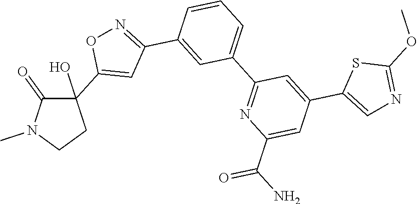 [SMILES: COc1ncc(-c2cc(C(N)=O)nc(-c3cccc(-c4cc(C5(O)CCN(C)C5=O)on4)c3)c2)s1]

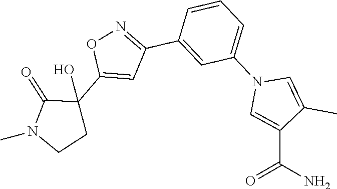

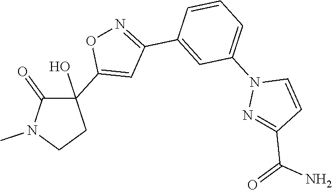 [SMILES: CN1CCC(O)(c2cc(-c3cccc(-n4ccc(C(N)=O)n4)c3)no2)C1=O]

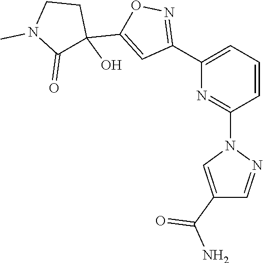 [SMILES: CN1CCC(O)(c2cc(-c3cccc(-n4cc(C(N)=O)cn4)n3)no2)C1=O]

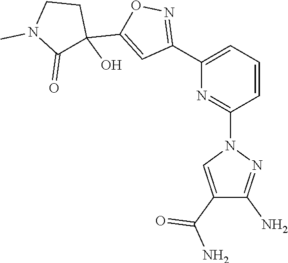 [SMILES: CN1CCC(O)(c2cc(-c3cccc(-n4cc(C(N)=O)c(N)n4)n3)no2)C1=O]

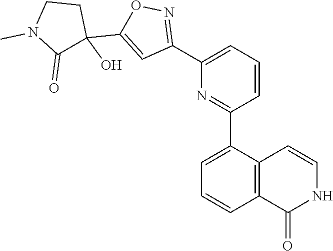 [SMILES: CN1CCC(O)(c2cc(-c3cccc(-c4cccc5c(=O)[nH]ccc45)n3)no2)C1=O]

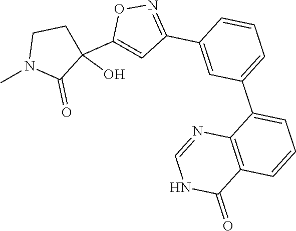 [SMILES: CN1CCC(O)(c2cc(-c3cccc(-c4cccc5c(=O)[nH]cnc45)c3)no2)C1=O]

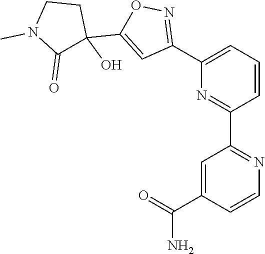 [SMILES: CN1CCC(O)(c2cc(-c3cccc(-c4cc(C(N)=O)ccn4)n3)no2)C1=O]

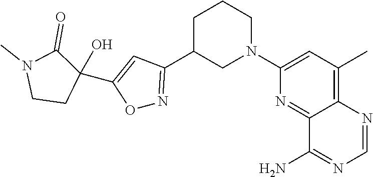

TABLE 1l

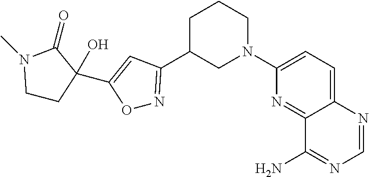 [SMILES: CN1CCC(O)(c2cc(C3CCCN(c4ccc5ncnc(N)c5n4)C3)no2)C1=O]

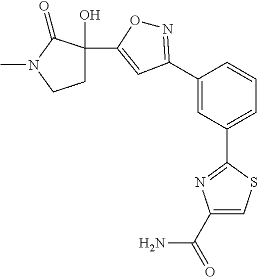 [SMILES: CN1CCC(O)(c2cc(-c3cccc(-c4nc(C(N)=O)cs4)c3)no2)C1=O]

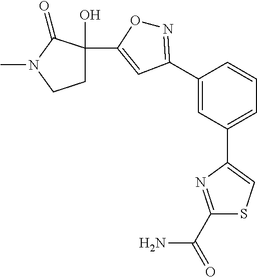 [SMILES: CN1CCC(O)(c2cc(-c3cccc(-c4csc(C(N)=O)n4)c3)no2)C1=O]

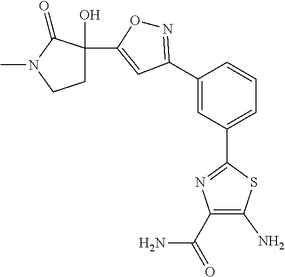 [SMILES: CN1CCC(O)(c2cc(-c3cccc(-c4nc(C(N)=O)c(N)s4)c3)no2)C1=O]

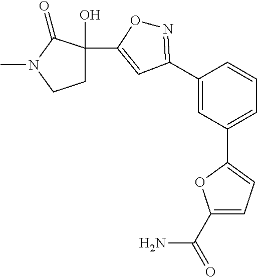 [SMILES: CN1CCC(O)(c2cc(-c3cccc(-c4ccc(C(N)=O)o4)c3)no2)C1=O]

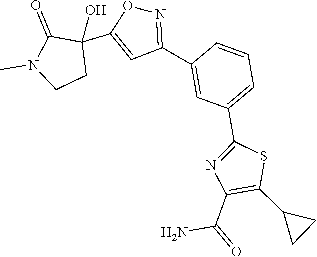 [SMILES: CN1CCC(O)(c2cc(-c3cccc(-c4nc(C(N)=O)c(C5CC5)s4)c3)no2)C1=O]

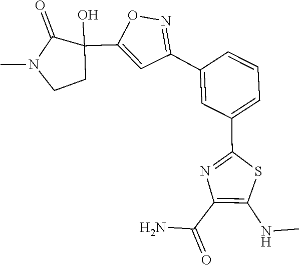

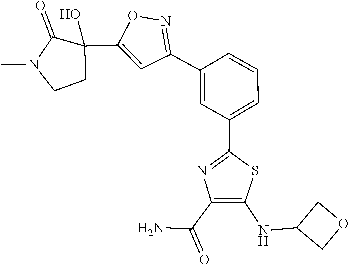 [SMILES: CN1CCC(O)(c2cc(-c3cccc(-c4nc(C(N)=O)c(NC5COC5)s4)c3)no2)C1=O]

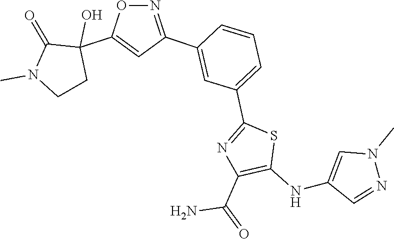

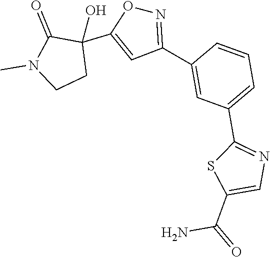 [SMILES: CN1CCC(O)(c2cc(-c3cccc(-c4ncc(C(N)=O)s4)c3)no2)C1=O]

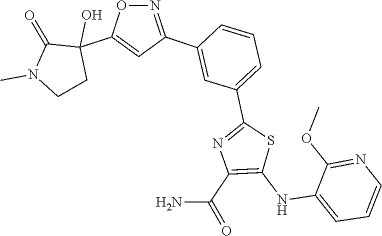 [SMILES: COc1ncccc1Nc1sc(-c2cccc(-c3cc(C4(O)CCN(C)C4=O)on3)c2)nc1C(N)=O]

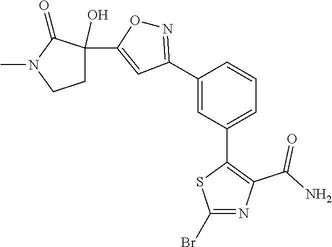 [SMILES: CN1CCC(O)(c2cc(-c3cccc(-c4sc(Br)nc4C(N)=O)c3)no2)C1=O]

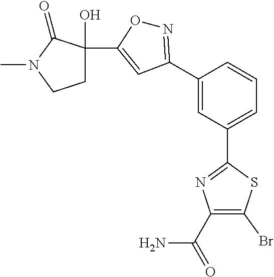 [SMILES: CN1CCC(O)(c2cc(-c3cccc(-c4nc(C(N)=O)c(Br)s4)c3)no2)C1=O]

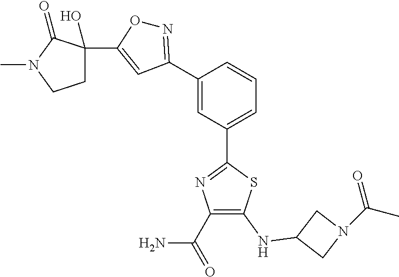 [SMILES: CC(=O)N1CC(Nc2sc(-c3cccc(-c4cc(C5(O)CCN(C)C5=O)on4)c3)nc2C(N)=O)C1]

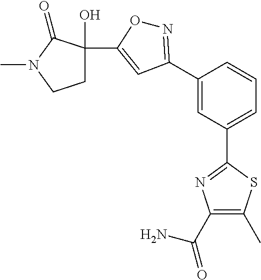

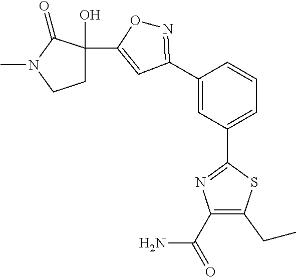 [SMILES: CCc1sc(-c2cccc(-c3cc(C4(O)CCN(C)C4=O)on3)c2)nc1C(N)=O]

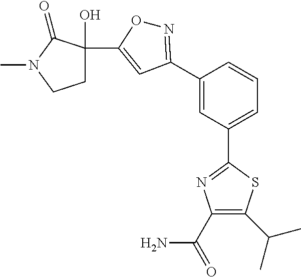 [SMILES: CC(C)c1sc(-c2cccc(-c3cc(C4(O)CCN(C)C4=O)on3)c2)nc1C(N)=O]

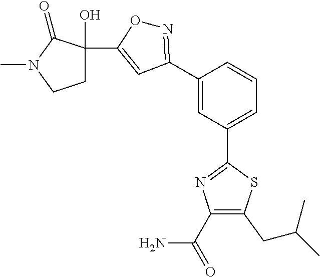 [SMILES: CC(C)Cc1sc(-c2cccc(-c3cc(C4(O)CCN(C)C4=O)on3)c2)nc1C(N)=O]

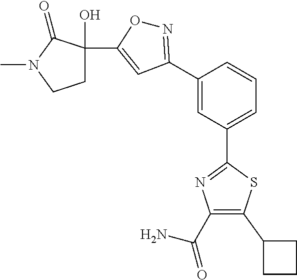 [SMILES: CN1CCC(O)(c2cc(-c3cccc(-c4nc(C(N)=O)c(C5CCC5)s4)c3)no2)C1=O]

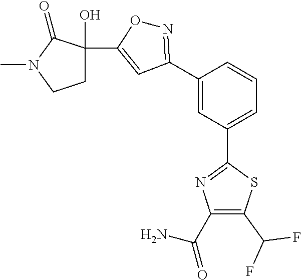 [SMILES: CN1CCC(O)(c2cc(-c3cccc(-c4nc(C(N)=O)c(C(F)F)s4)c3)no2)C1=O]

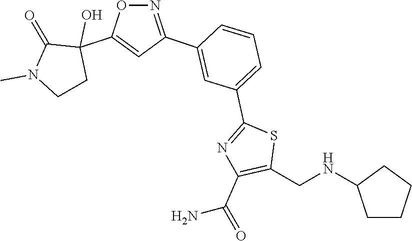 [SMILES: CN1CCC(O)(c2cc(-c3cccc(-c4nc(C(N)=O)c(CNC5CCCC5)s4)c3)no2)C1=O]

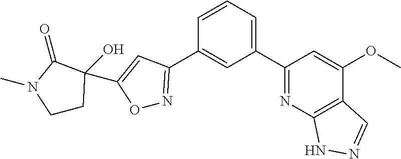

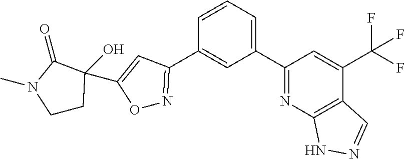 [SMILES: CN1CCC(O)(c2cc(-c3cccc(-c4cc(C(F)(F)F)c5cn[nH]c5n4)c3)no2)C1=O]

TABLE 1J

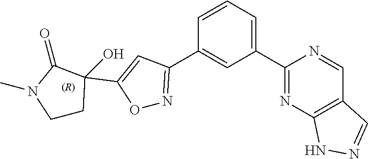

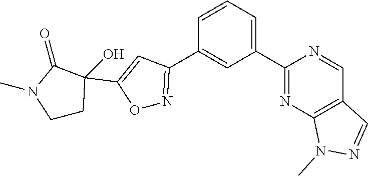 [SMILES: CN1CCC(O)(c2cc(-c3cccc(-c4ncc5cnn(C)c5n4)c3)no2)C1=O]

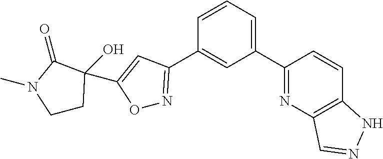 [SMILES: CN1CCC(O)(c2cc(-c3cccc(-c4ccc5[nH]ncc5n4)c3)no2)C1=O]

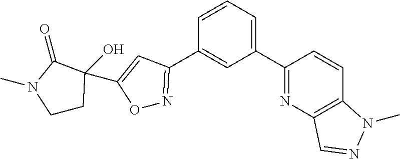

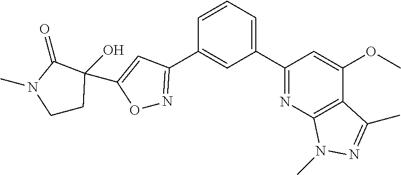

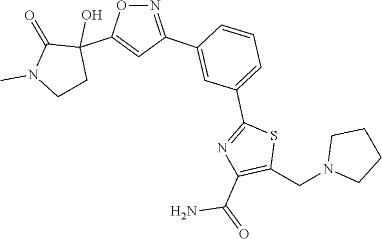 [SMILES: CN1CCC(O)(c2cc(-c3cccc(-c4nc(C(N)=O)c(CN5CCCC5)s4)c3)no2)C1=O]

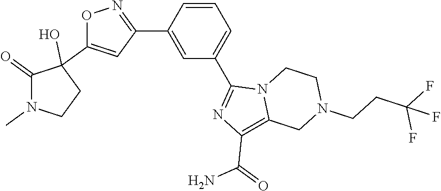 [SMILES: CN1CCC(O)(c2cc(-c3cccc(-c4nc(C(N)=O)c5n4CCN(CCC(F)(F)F)C5)c3)no2)C1=O]

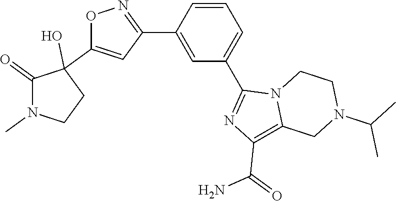 [SMILES: CC(C)N1CCn2c(-c3cccc(-c4cc(C5(O)CCN(C)C5=O)on4)c3)nc(C(N)=O)c2C1]

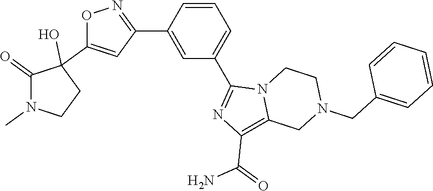 [SMILES: CN1CCC(O)(c2cc(-c3cccc(-c4nc(C(N)=O)c5n4CCN(Cc4ccccc4)C5)c3)no2)C1=O]

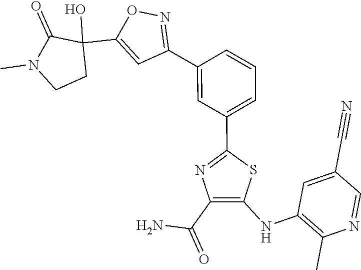 [SMILES: Cc1ncc(C#N)cc1Nc1sc(-c2cccc(-c3cc(C4(O)CCN(C)C4=O)on3)c2)nc1C(N)=O]

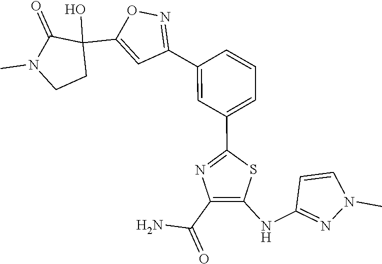

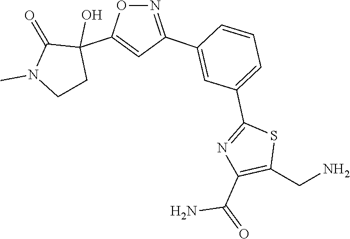 [SMILES: CN1CCC(O)(c2cc(-c3cccc(-c4nc(C(N)=O)c(CN)s4)c3)no2)C1=O]

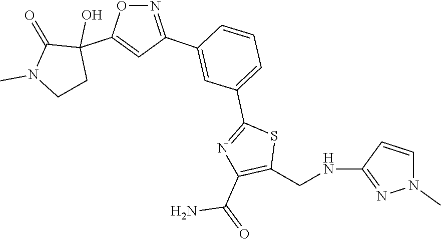

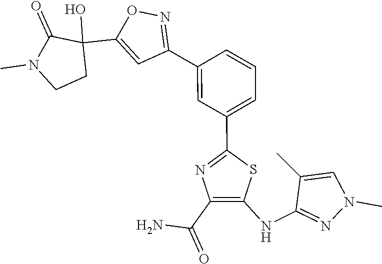

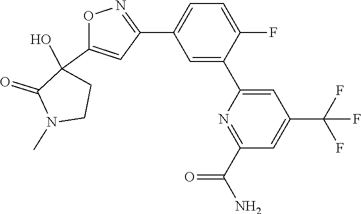 [SMILES: CN1CCC(O)(c2cc(-c3ccc(F)c(-c4cc(C(F)(F)F)cc(C(N)=O)n4)c3)no2)C1=O]

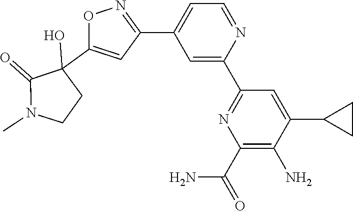 [SMILES: CN1CCC(O)(c2cc(-c3ccnc(-c4cc(C5CC5)c(N)c(C(N)=O)n4)c3)no2)C1=O]

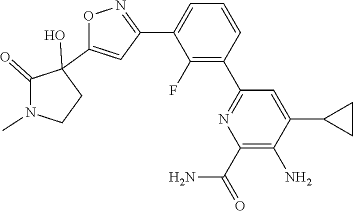 [SMILES: CN1CCC(O)(c2cc(-c3cccc(-c4cc(C5CC5)c(N)c(C(N)=O)n4)c3F)no2)C1=O]

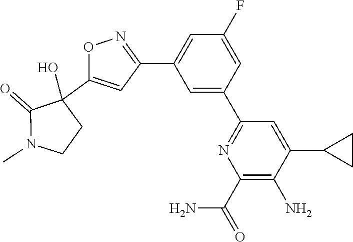 [SMILES: CN1CCC(O)(c2cc(-c3cc(F)cc(-c4cc(C5CC5)c(N)c(C(N)=O)n4)c3)no2)C1=O]

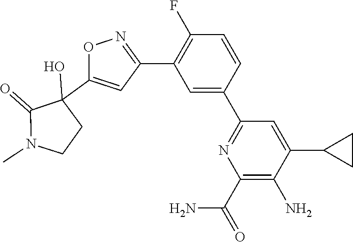 [SMILES: CN1CCC(O)(c2cc(-c3cc(-c4cc(C5CC5)c(N)c(C(N)=O)n4)ccc3F)no2)C1=O]

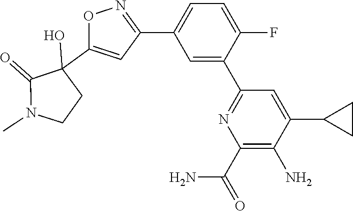 [SMILES: CN1CCC(O)(c2cc(-c3ccc(F)c(-c4cc(C5CC5)c(N)c(C(N)=O)n4)c3)no2)C1=O]

In some embodiments, disclosed herein is a compound of Formula I, or a pharmaceutically acceptable salt thereof, having a structure as shown in any one of Tables 2A to 2J.

TABLE 2A

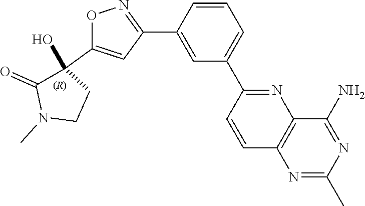

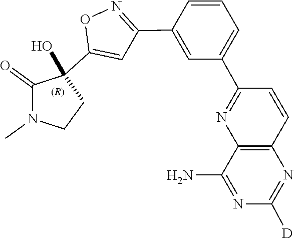

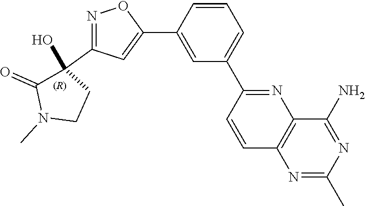

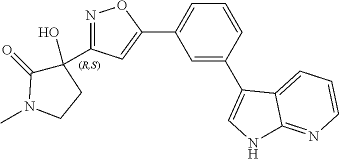 [SMILES: CN1CCC(O)(c2cc(-c3cccc(-c4c[nH]c5ncccc45)c3)on2)C1=O]

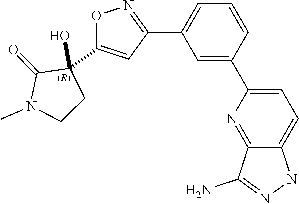

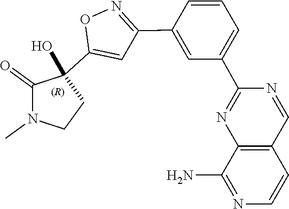

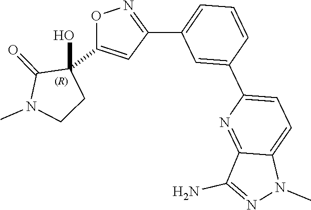

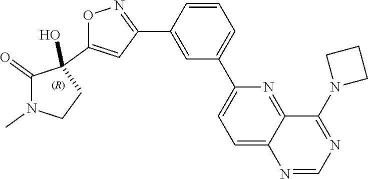

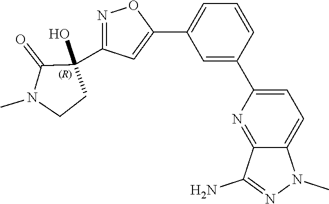

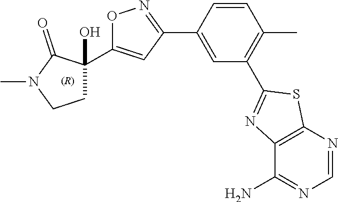

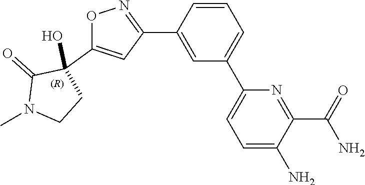

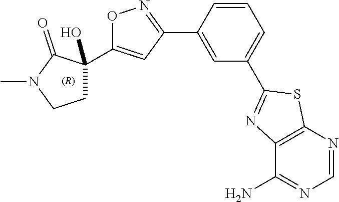

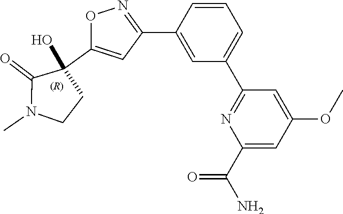

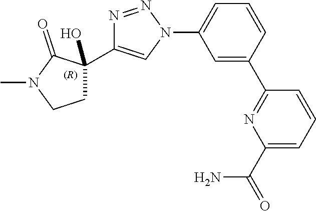

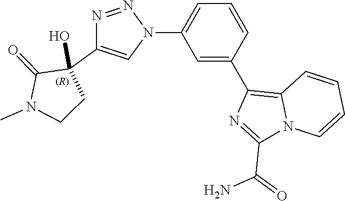

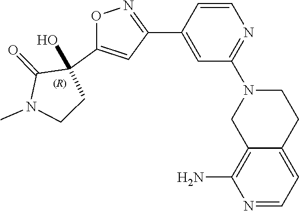

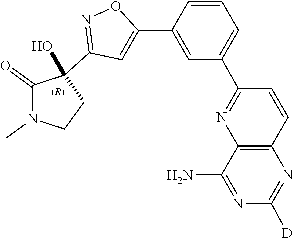

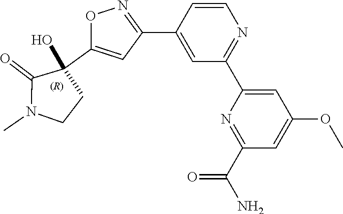

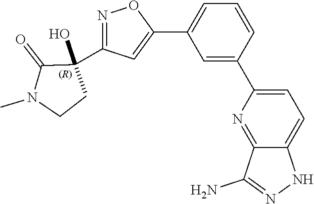

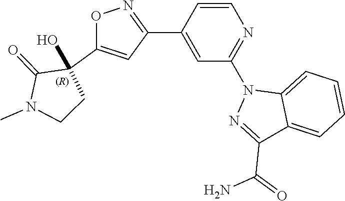

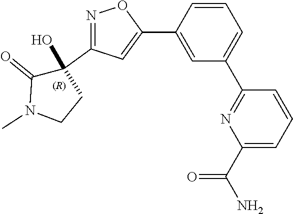

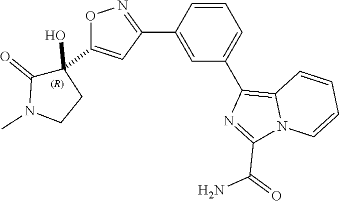

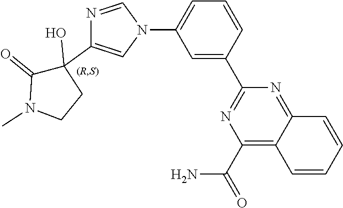

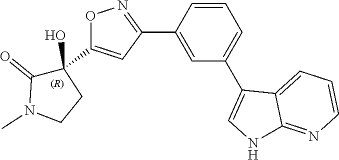

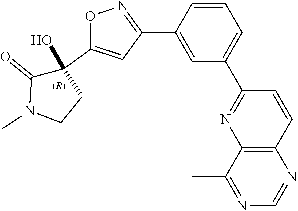

TABLE 2B

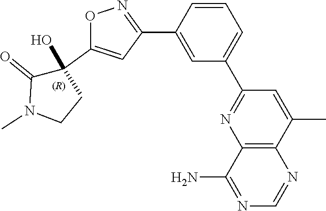

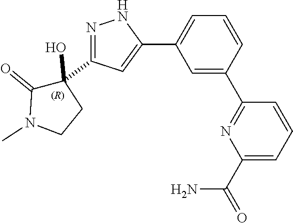

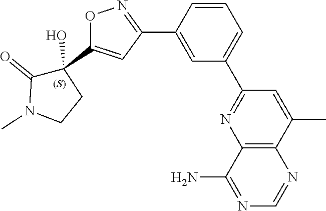

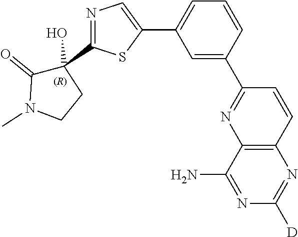

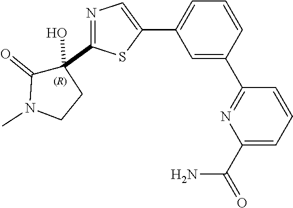

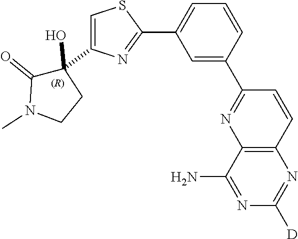

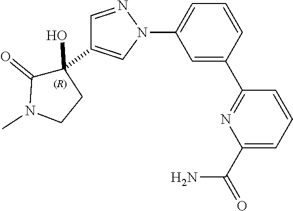

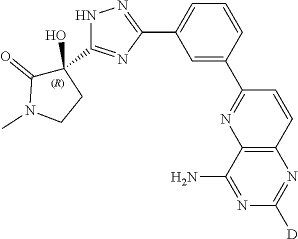

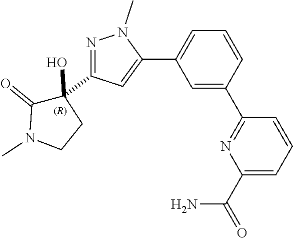

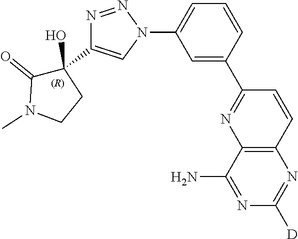

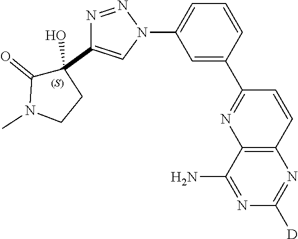

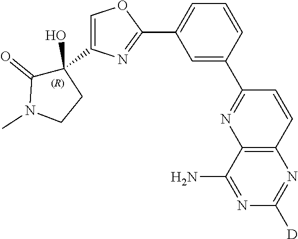

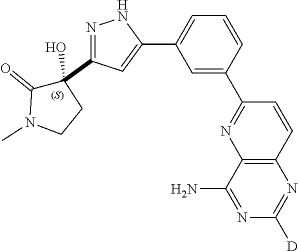

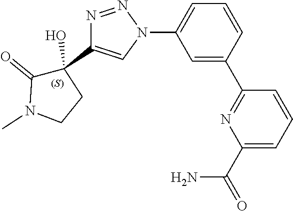

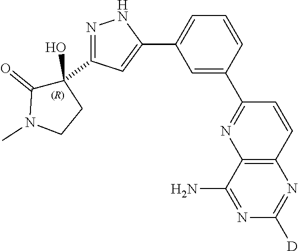

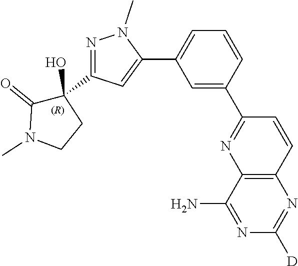

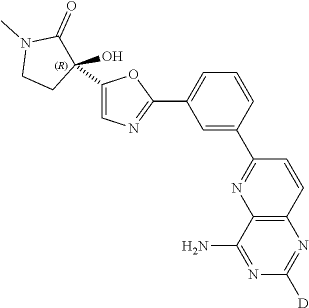

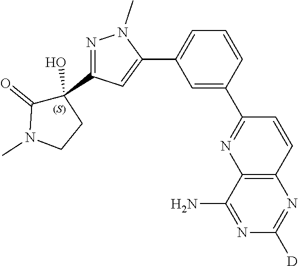

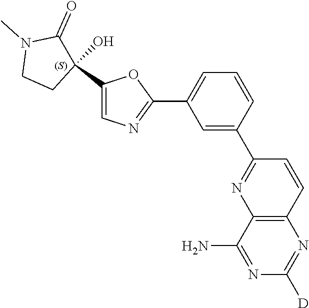

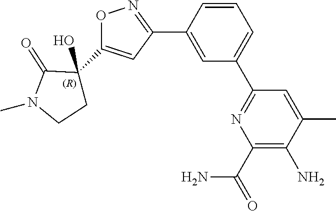

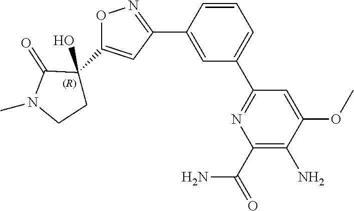

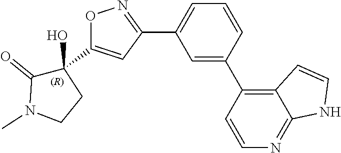

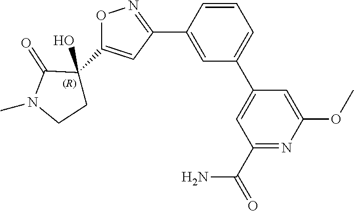

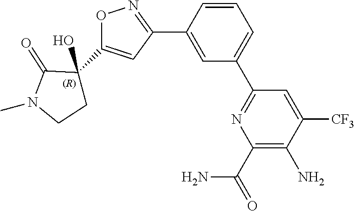

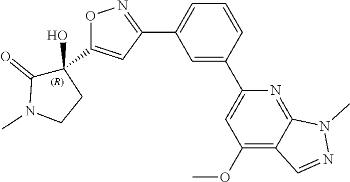

TABLE 2C

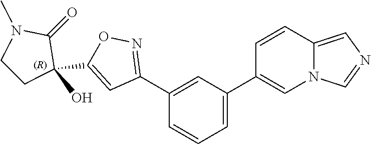

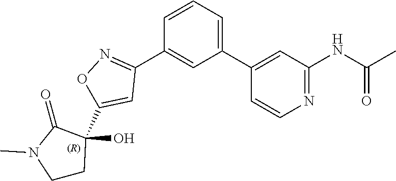

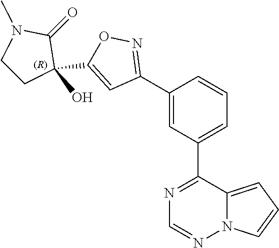

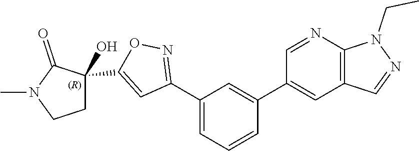

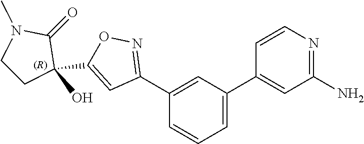

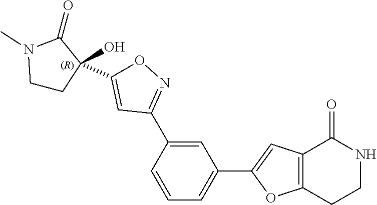

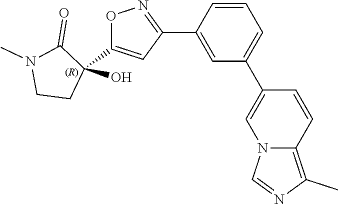

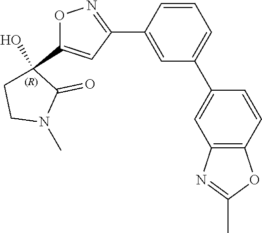

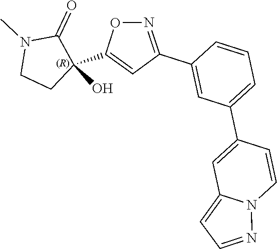

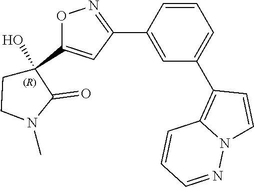

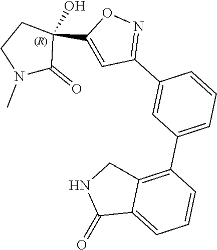

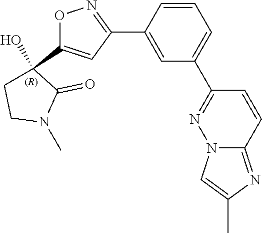

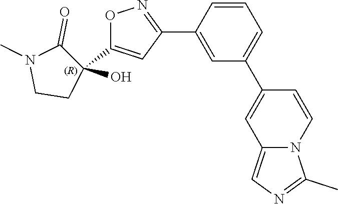

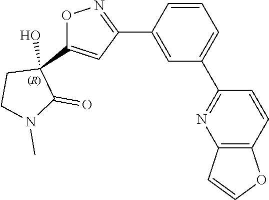

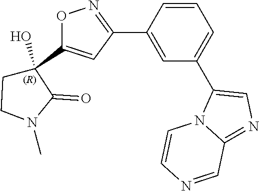

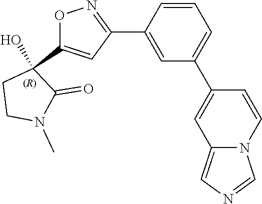

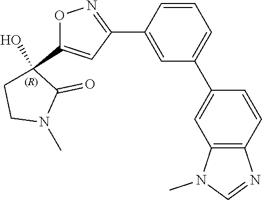

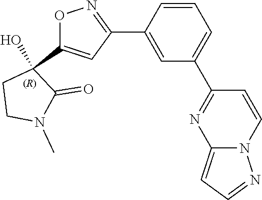

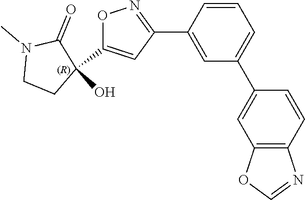

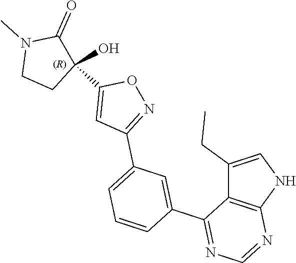

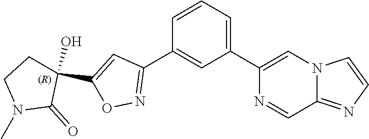

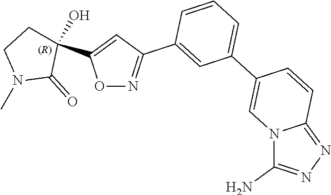

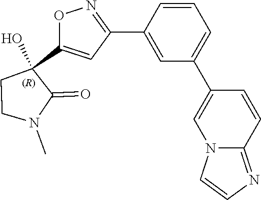

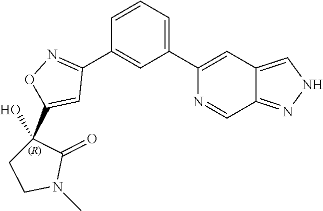

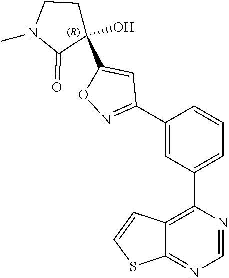

TABLE 2D

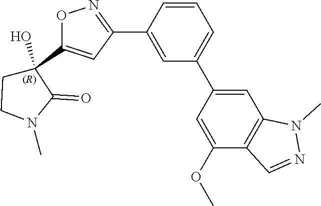

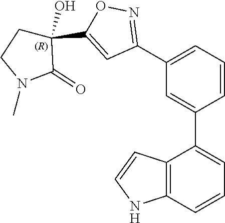

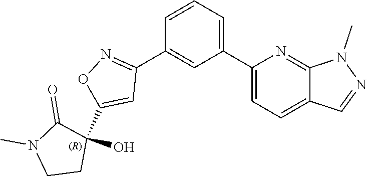

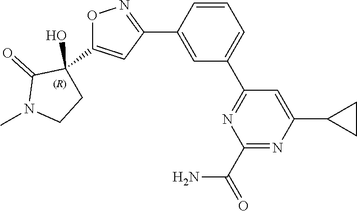

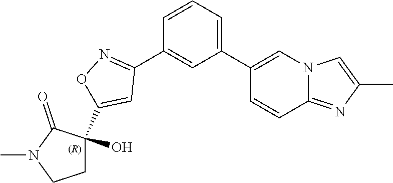

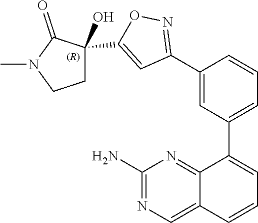

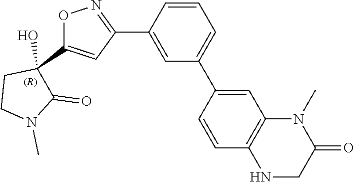

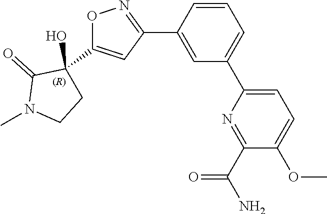

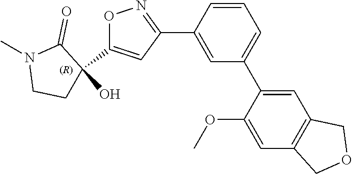

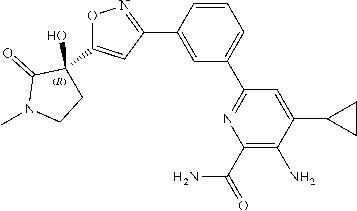

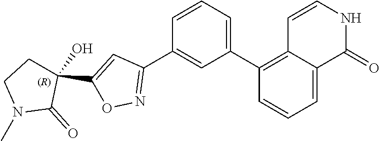

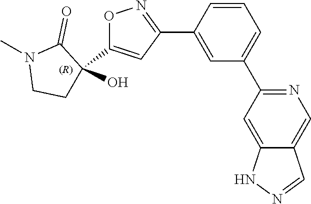

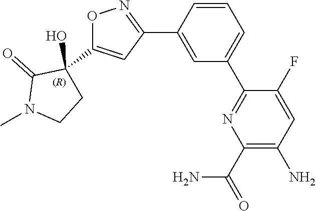

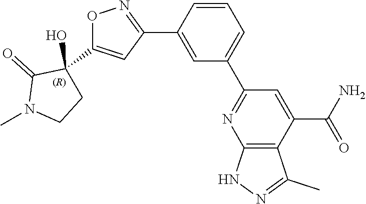

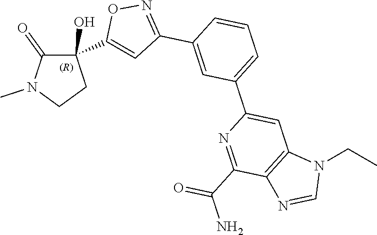

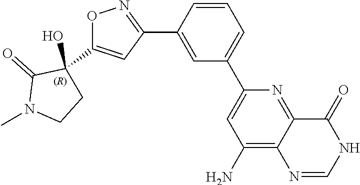

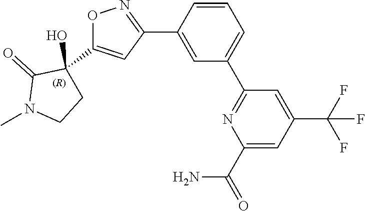

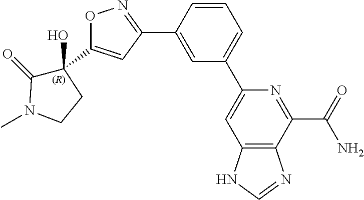

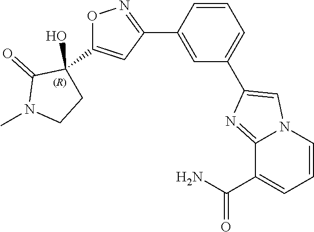

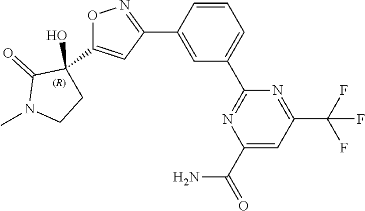

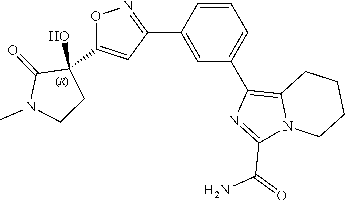

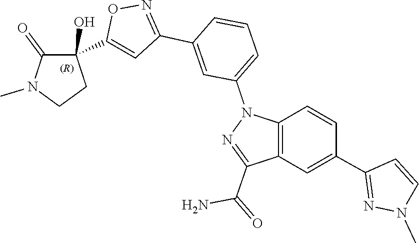

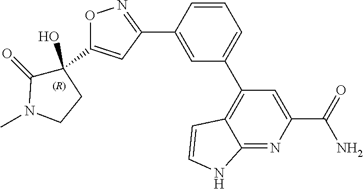

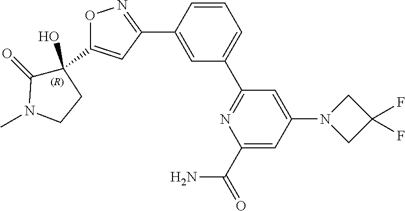

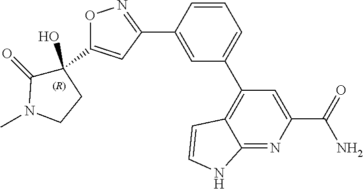

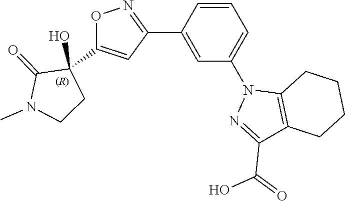

TABLE 2E

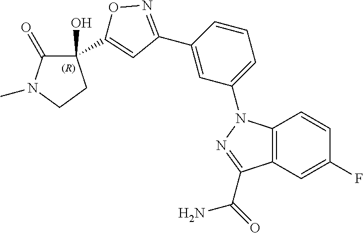

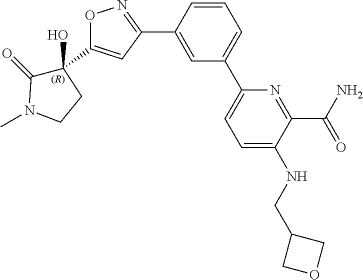

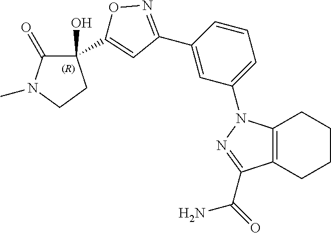

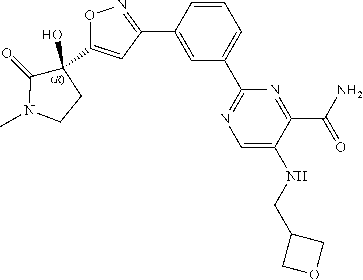

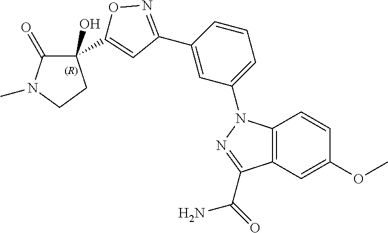

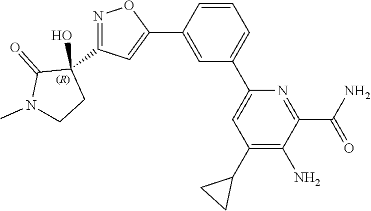

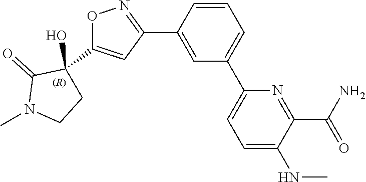

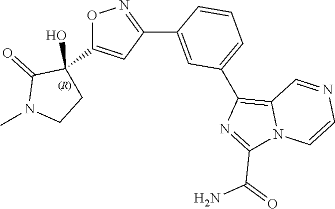

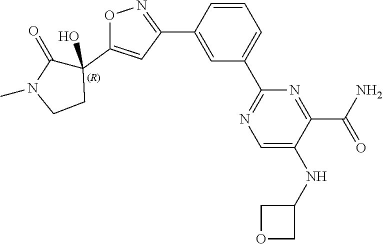

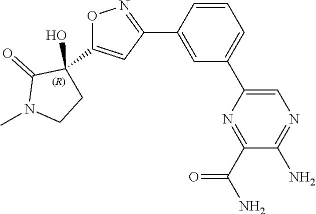

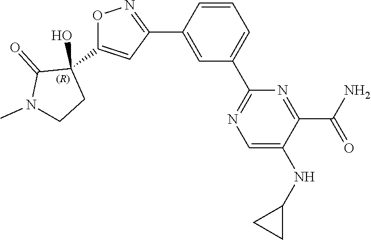

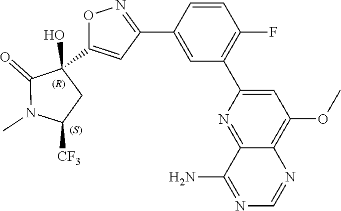

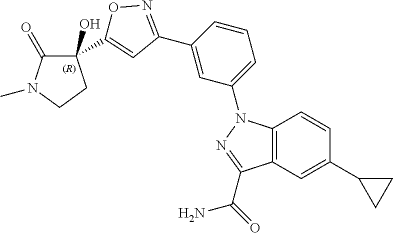

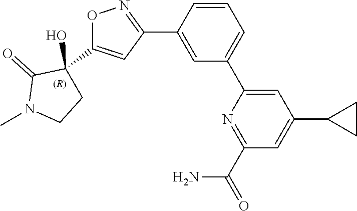

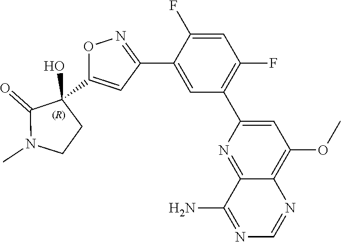

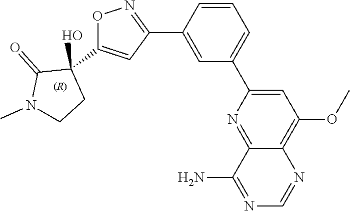

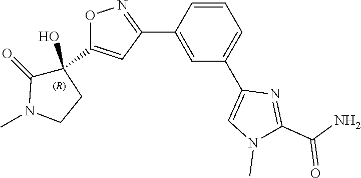

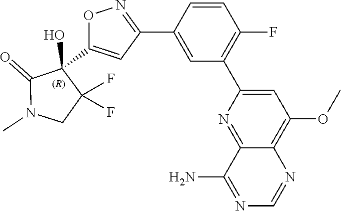

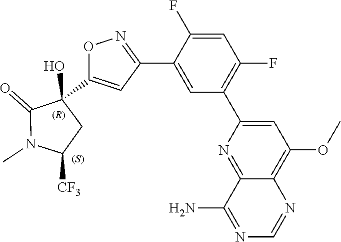

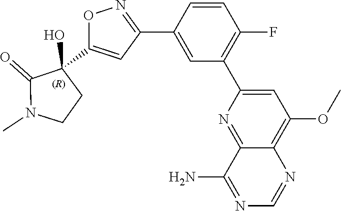

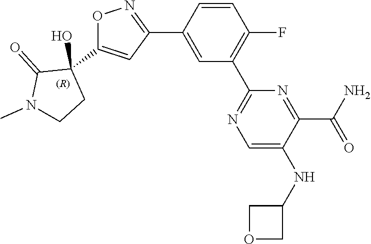

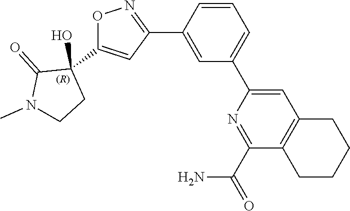

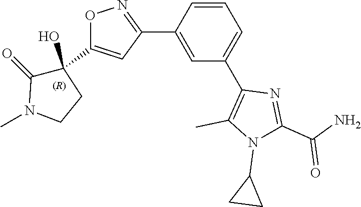

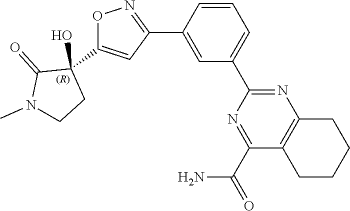

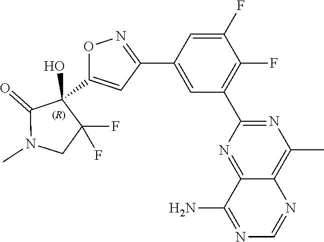

TABLE 2F

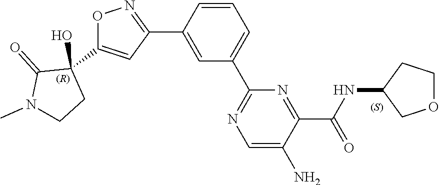

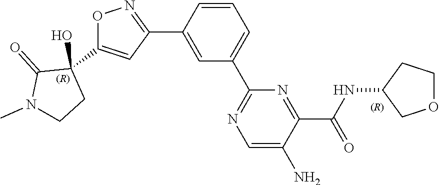

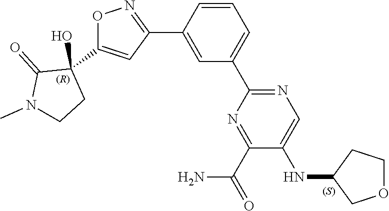

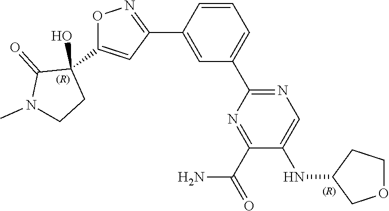

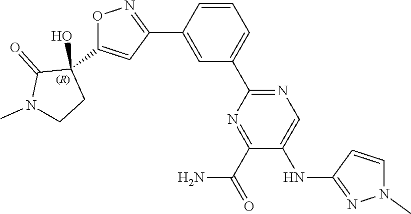

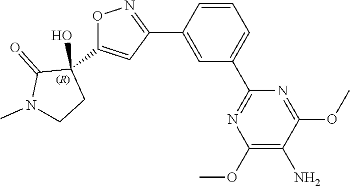

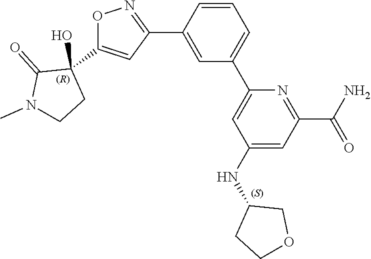

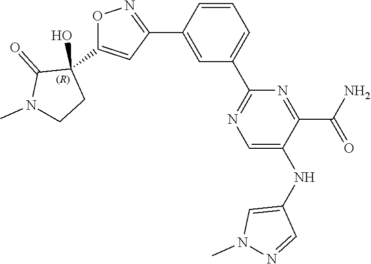

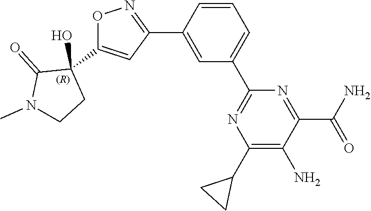

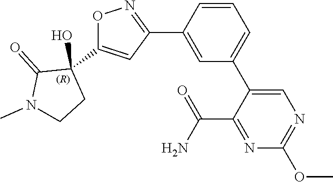

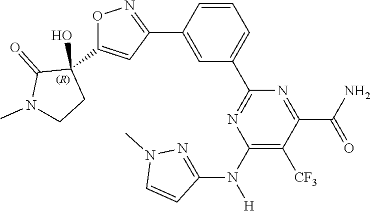

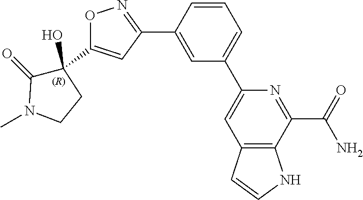

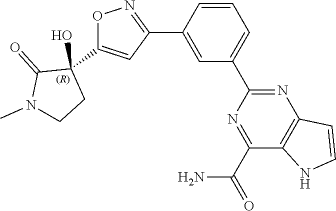

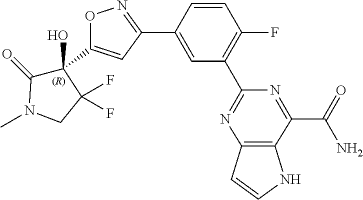

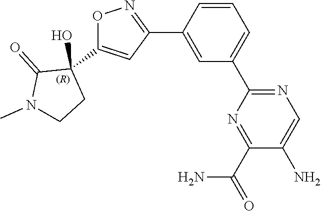

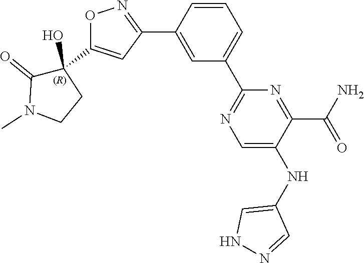

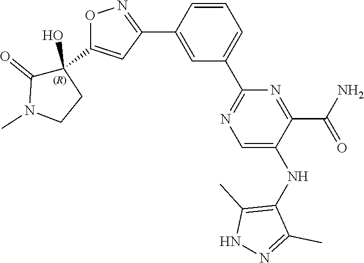

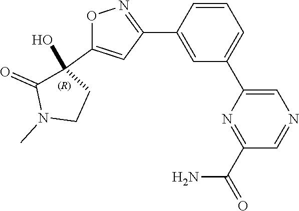

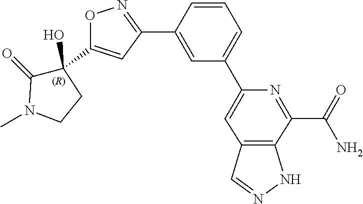

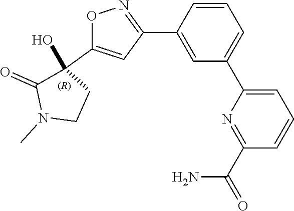

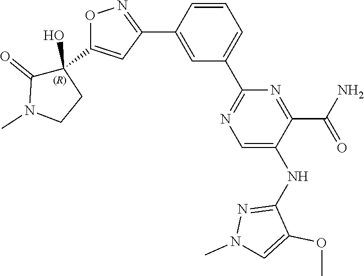

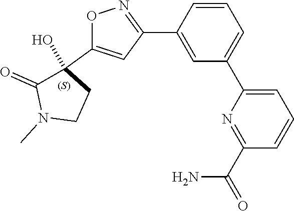

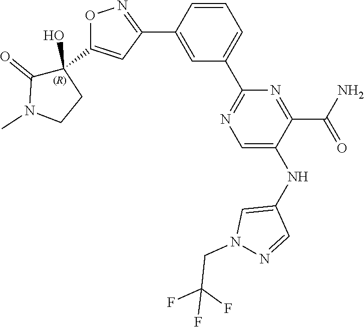

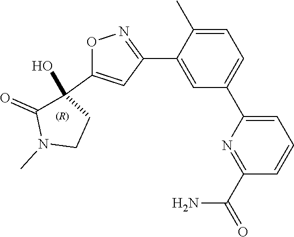

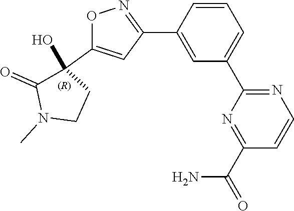

TABLE 2G

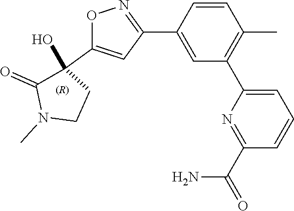

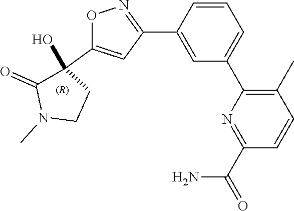

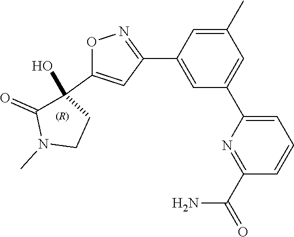

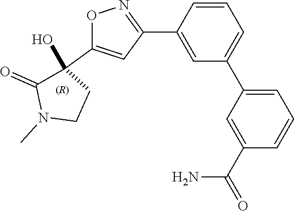

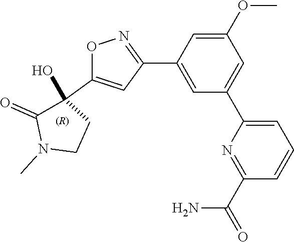

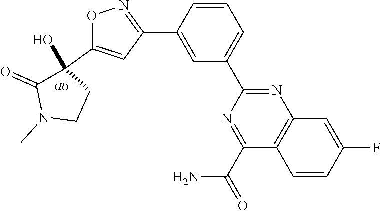

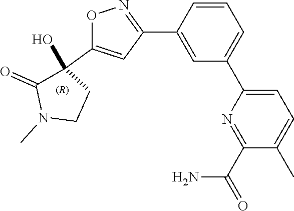

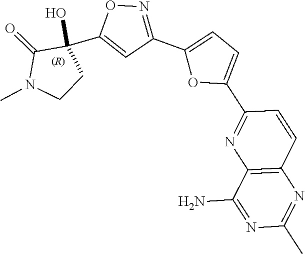

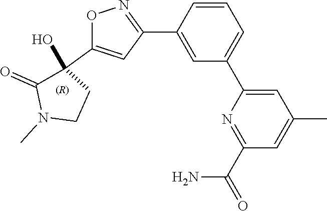

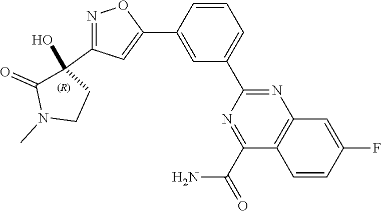

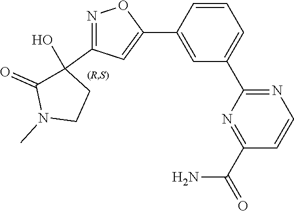

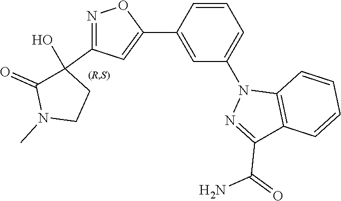

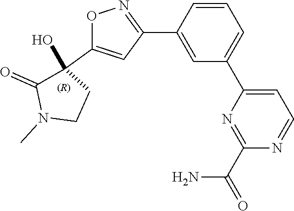

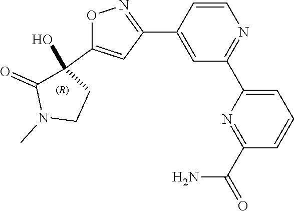

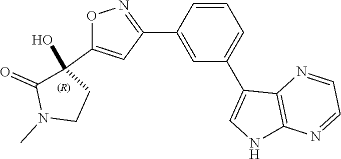

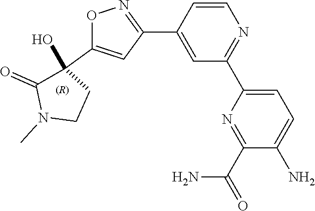

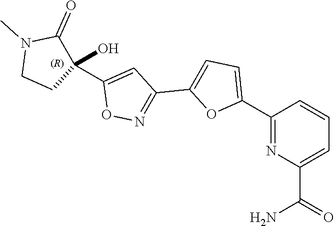

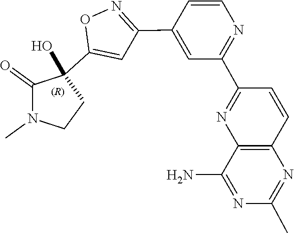

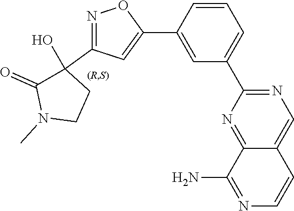

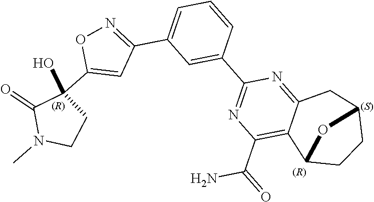

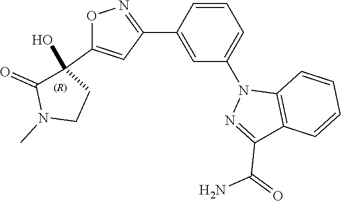

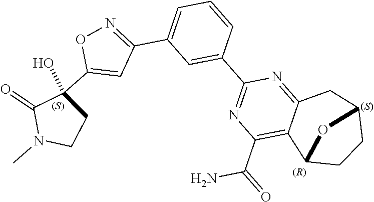

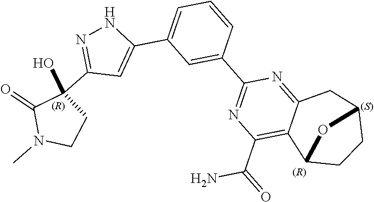

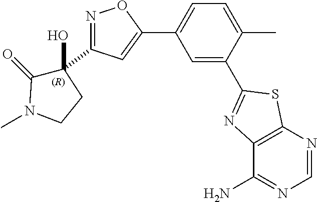

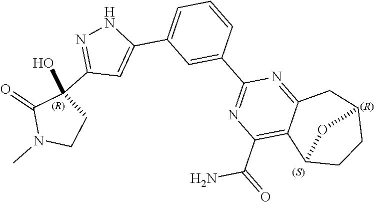

TABLE 2H

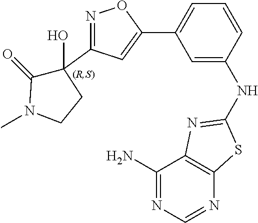

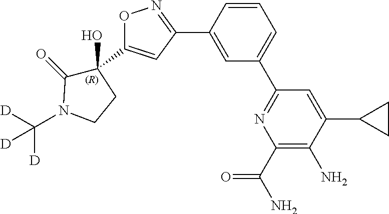

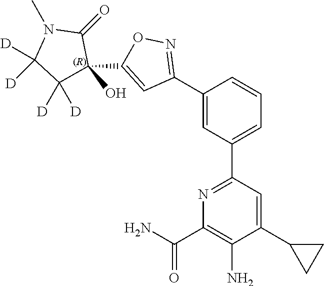

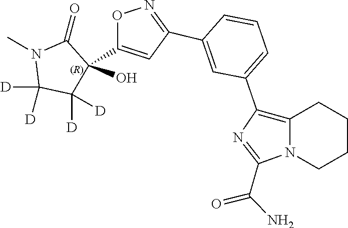

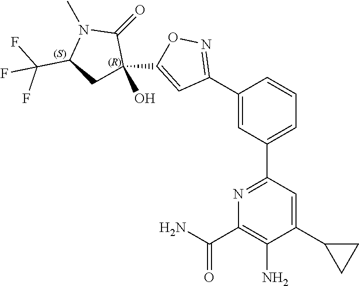

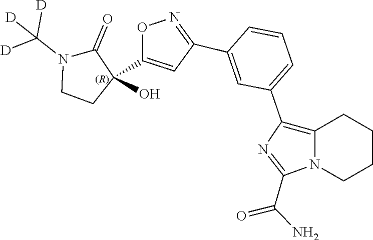

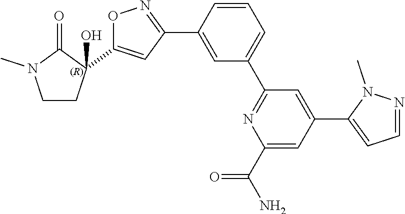

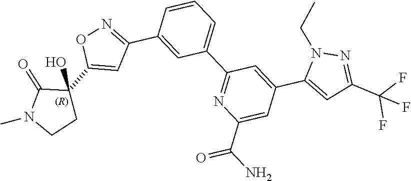

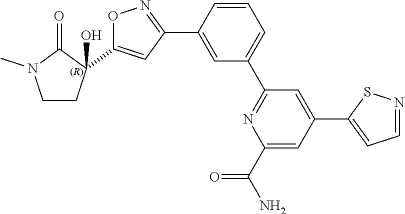

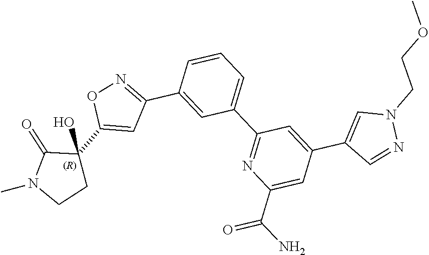

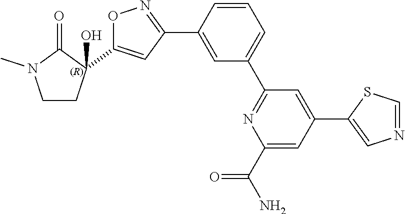

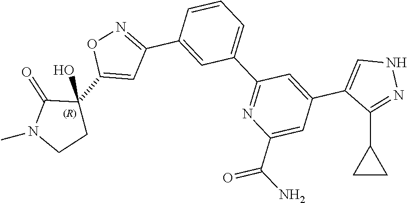

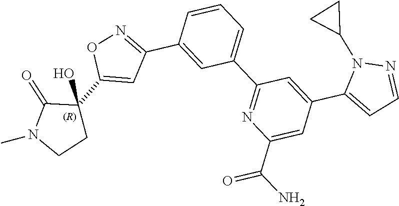

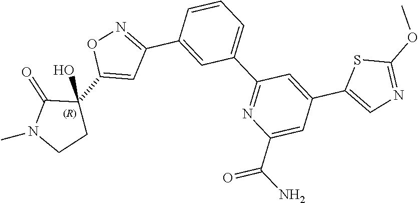

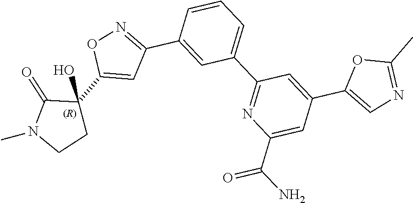

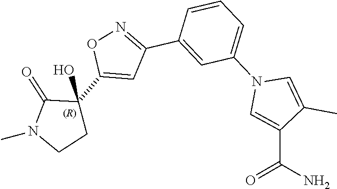

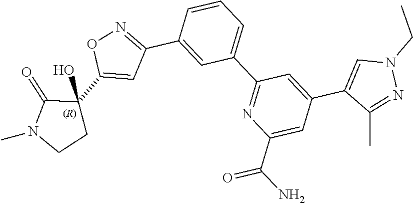

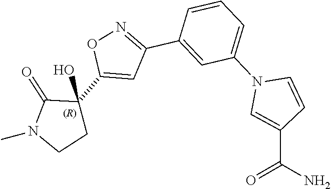

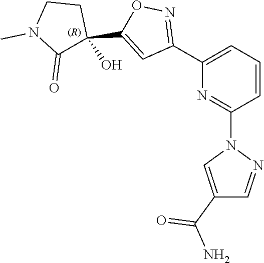

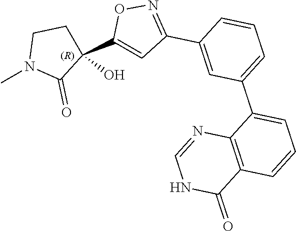

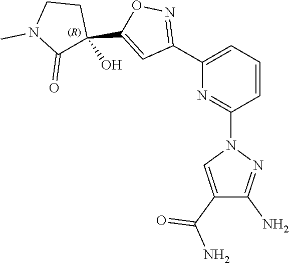

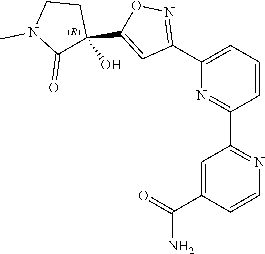

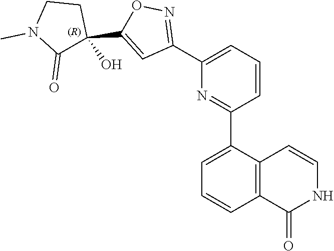

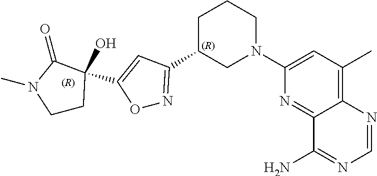

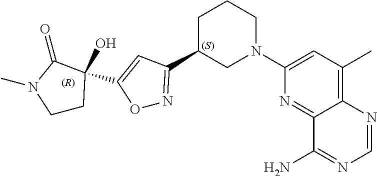

TABLE 2I

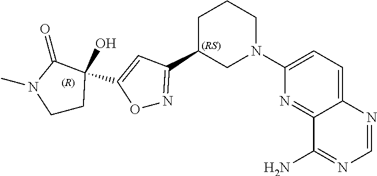

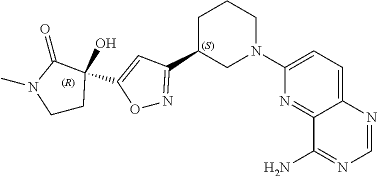

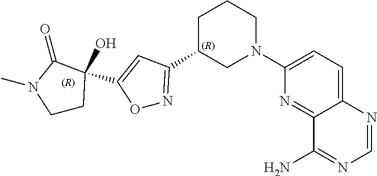

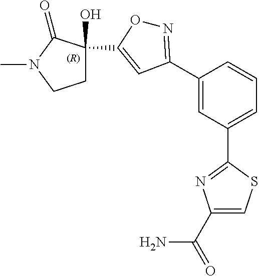

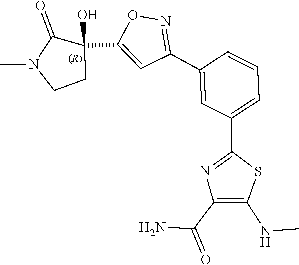

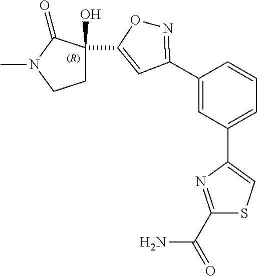

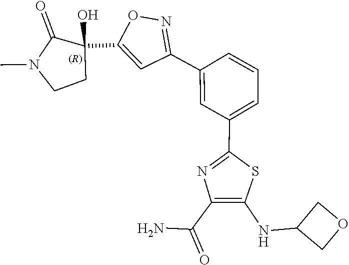

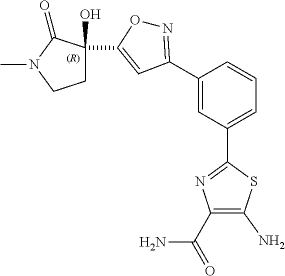

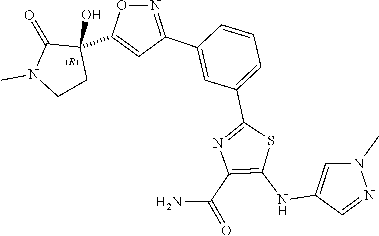

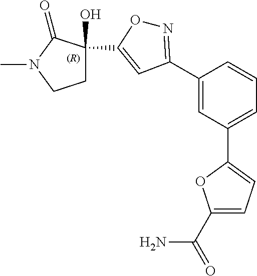

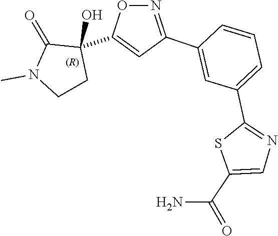

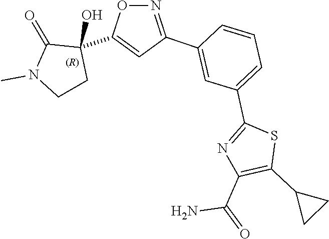

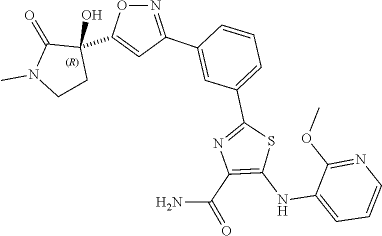

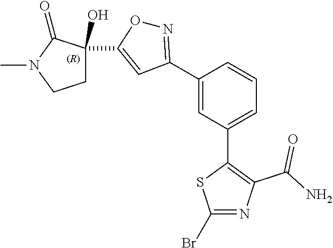

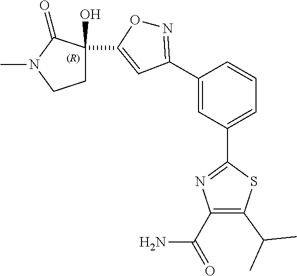

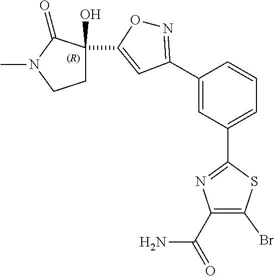

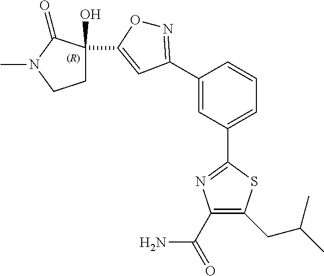

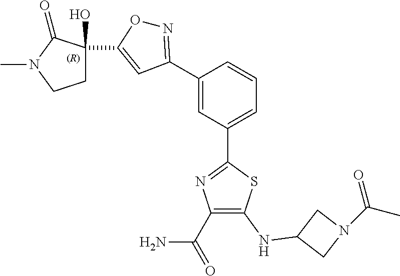

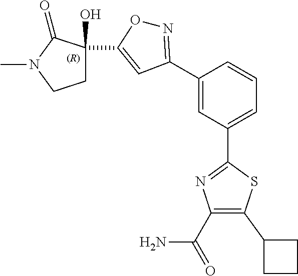

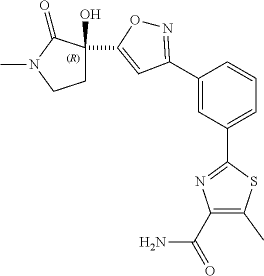

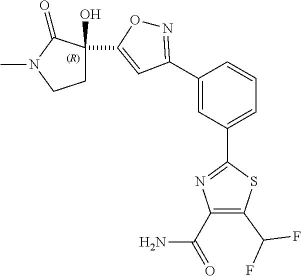

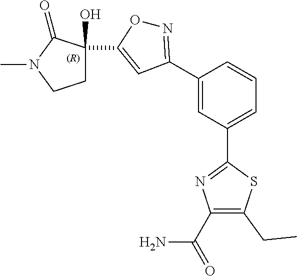

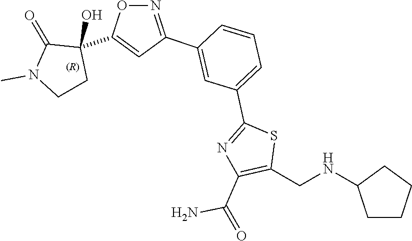

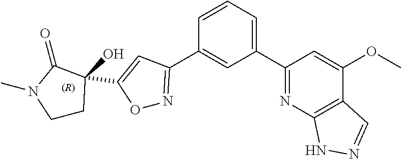

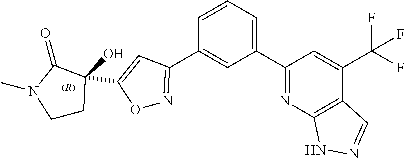

TABLE 2J

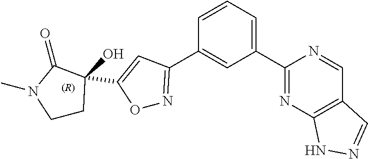

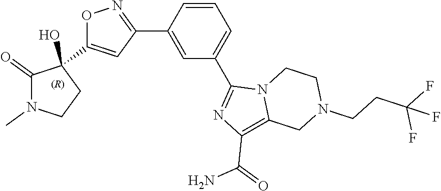

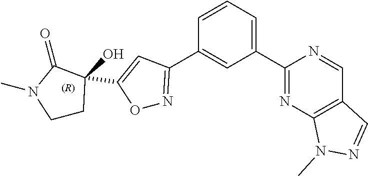

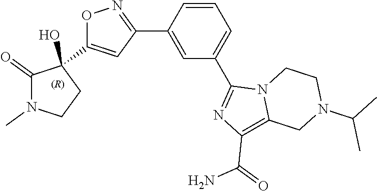

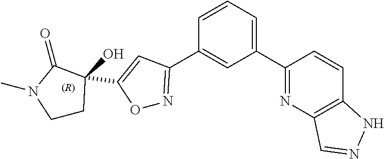

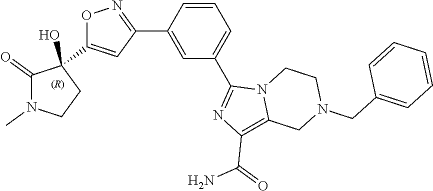

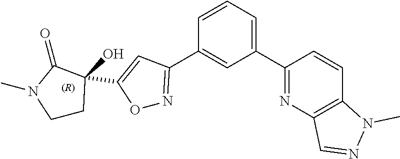

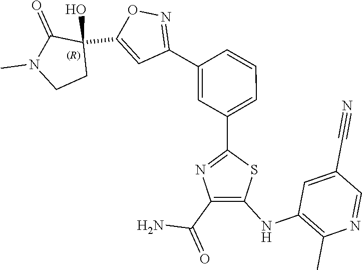

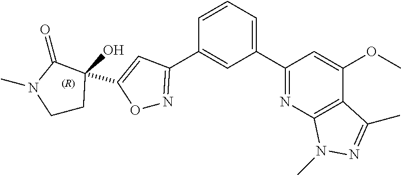

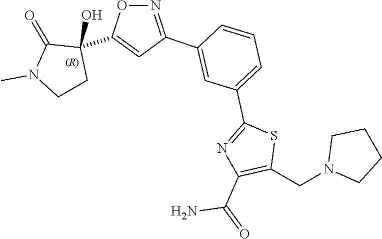

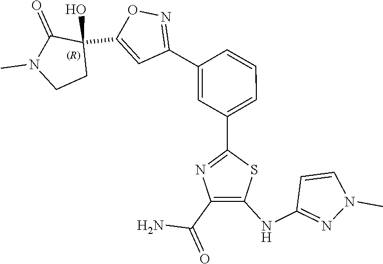

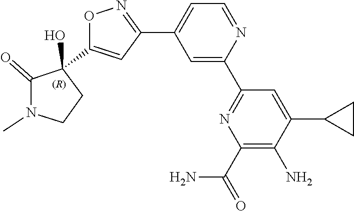

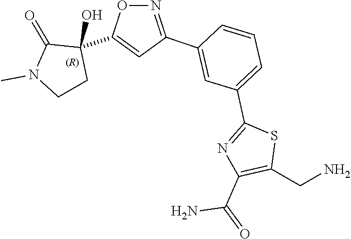

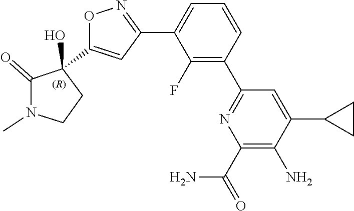

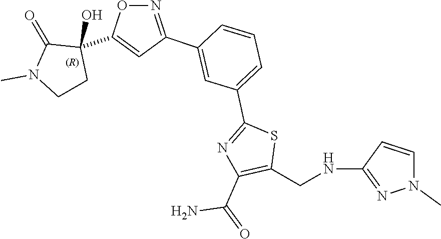

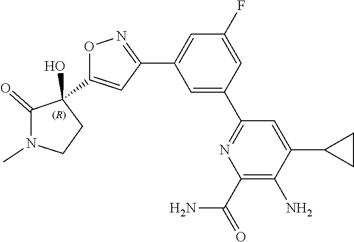

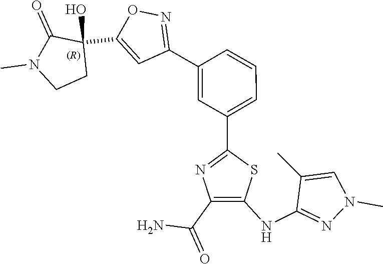

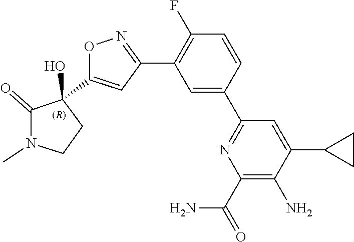

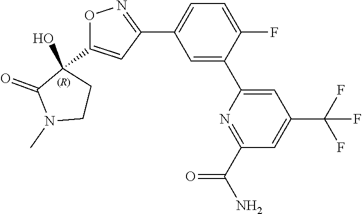

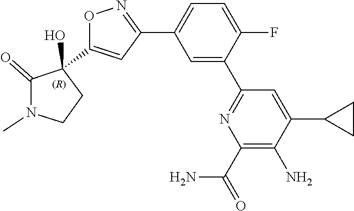

In some embodiments, disclosed herein is a compound of Formula I, or a pharmaceutically acceptable salt thereof, selected from the group consisting of:

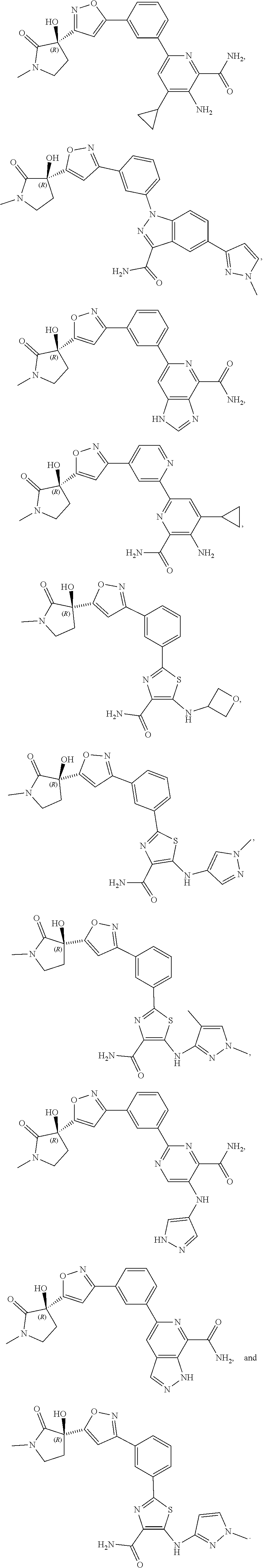

In some embodiments, disclosed herein is a compound of Formula I having the following structure:

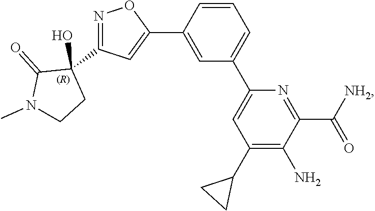

or a pharmaceutically acceptable salt thereof.

In some embodiments, disclosed herein is a compound of Formula I having the following structure:

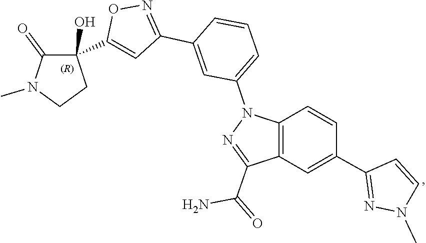

or a pharmaceutically acceptable salt thereof.

In some embodiments, disclosed herein is a compound of Formula I having the following structure:

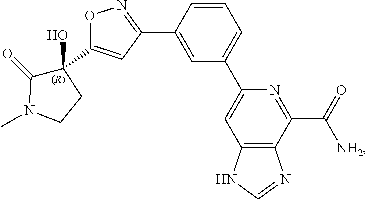

or a pharmaceutically acceptable salt thereof.

In some embodiments, disclosed herein is a compound of Formula I having the following structure:

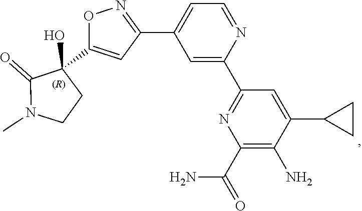

or a pharmaceutically acceptable salt thereof.

In some embodiments, disclosed herein is a compound of Formula I having the following structure:

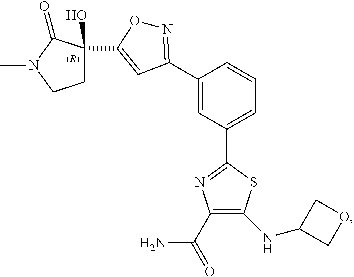

or a pharmaceutically acceptable salt thereof.

In some embodiments, disclosed herein is a compound of Formula I having the following structure:

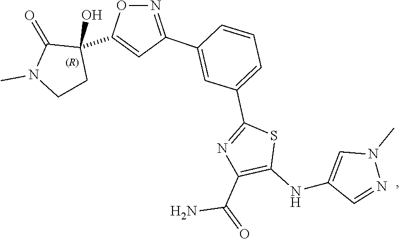

or a pharmaceutically acceptable salt thereof.

In some embodiments, disclosed herein is a compound of Formula I having the following structure:

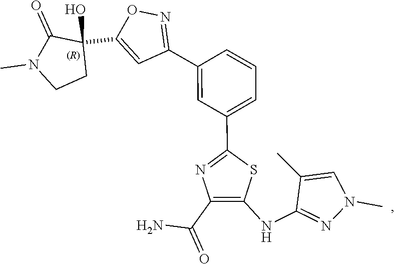

or a pharmaceutically acceptable salt thereof.

In some embodiments, disclosed herein is a compound of Formula I having the following structure:

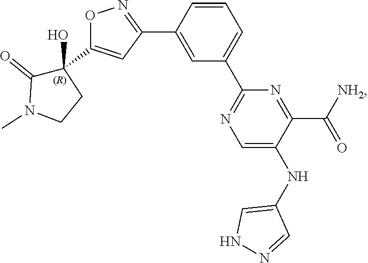

or a pharmaceutically acceptable salt thereof.

In some embodiments, disclosed herein is a compound of Formula I having the following structure:

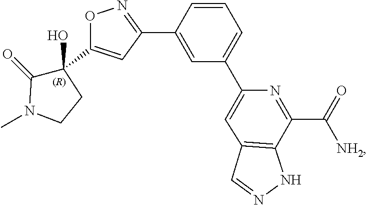

or a pharmaceutically acceptable salt thereof.

In some embodiments, disclosed herein is a compound of Formula I having the following structure:

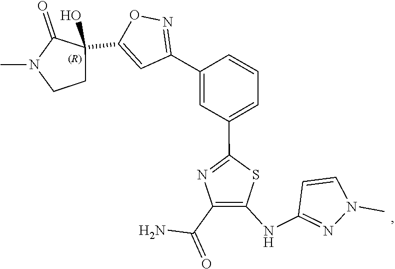

or a pharmaceutically acceptable salt thereof.

In some embodiments, disclosed herein is a pharmaceutical composition comprising a compound of Formula I, or a pharmaceutically acceptable salt thereof, and a pharmaceutically acceptable carrier.

### Therapeutic Use

The present disclosure is also directed toward a method for treating a disease, disorder, or medical condition mediated by NIK activity, comprising administering to a subject in need of such treatment an effective amount of a compound of the disclosure, or a pharmaceutically acceptable salt thereof, or a pharmaceutical composition of the disclosure.

The present disclosure is also directed toward a method for preventing a disease, disorder, or medical condition mediated by NIK activity, comprising administering to a subject in need of such treatment an effective amount of a compound of the disclosure, or a pharmaceutically acceptable salt thereof, or a pharmaceutical composition of the disclosure.

The present disclosure is also directed toward a method for improving or ameliorating a symptom of a disease, disorder, or medical condition mediated by NIK activity, comprising administering to a subject in need of such treatment an effective amount of a compound of the disclosure, or a pharmaceutically acceptable salt thereof, or a pharmaceutically acceptable salt thereof. In some embodiments, the compound described herein can be administered in combination with one or more additional therapeutic agent(s).

In some embodiments of the methods of treatment disclosed herein, the disease, disorder, or medical condition is selected from the group consisting of inflammatory disorders and autoimmune disorders.

In some embodiments, the disease, disorder, or medical condition is selected from the group consisting of systemic lupus erythematosus, rheumatoid arthritis, Sjogren's syndrome, and lupus nephritis.

In some embodiments of the methods of treatment disclosed herein, the disease, disorder, or medical condition is selected from the group consisting of inflammatory disorders, autoimmune disorders, cancers, metabolic disorders, and osteoporosis.

In some embodiments of the methods of treatment disclosed herein, the disease, disorder, or medical condition is an autoantibody associated disease. In some embodiments, the autoantibody associated diseases is selected from the group consisting of anti-neutrophil cytoplasmic antibody (“ANCA”) associated vasculitis, scleroderma, Sjogren's disease, myositis, IgG4 associated diseases, bullous pemphigoid, and neuromyelitis optica spectrum disorders (“NMOSD”).

In some embodiments of the methods of treatment disclosed herein, the disease, disorder, or medical condition is an immune mediated dermatitis indication. In some embodiments, the immune mediated dermatitis indication is selected from the group consisting of atopic dermatitis and hidradenitis supperativa.

In some embodiments of the methods of treatment disclosed herein, the disease, disorder, or medical condition is a liver inflammation or a liver injury. In some embodiments, the liver inflammation or a liver injury is selected from the group consisting of steatosis, non-alcoholic steatohepatitis (“NASH”) and primary biliary cirrhosis.

In some embodiments of the methods of treatment disclosed herein, the disease, disorder, or medical condition is cancer. In some embodiments, the cancer is selected from the group consisting of leukemias, lymphomas, pancreatic cancer, breast cancer, and melanoma.

In some embodiments of the methods of treatment disclosed herein, the disease, disorder, or medical condition is a metabolic disorder. In some embodiments, the metabolic disorder is selected from the group consisting of obesity and diabetes. In some embodiments, the diabetes is a type 2 diabetes.

In some embodiments of the methods of treatment disclosed herein, the disease, disorder, or medical condition is a kidney disease. In some embodiments, the kidney disease is selected from the group consisting of acute kidney injury, Berger's disease (IgA nephropathy (IgAN)), autosomal dominant polycystic kidney disease (“ADCKD”), and membranous nephropathy.

In some embodiments of the methods of treatment disclosed herein, the disease, disorder, or medical condition is osteoporosis.

In some embodiments of the methods of treatment disclosed herein, the disease, disorder, or medical condition is selected from the group consisting of RA, IBD, SLE, IgAN, metabolic syndrome, multiple sclerosis, Immune thrombocytopenic purpura, primary biliary cirrhosis, transplantation, myasthenia gravis, osteoporosis, and bone resorption (periodontitis).

In some embodiments of the methods of treatment disclosed herein, the disease, disorder, or medical condition is selected from the group consisting of systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.

In some embodiments, disclosed herein is a method for preventing or controlling an excessive inflammatory response, comprising administering to a subject in need of such treatment an effective amount of a compound of the disclosure, or a pharmaceutically acceptable salt thereof, or a pharmaceutical composition of the disclosure.

In some embodiments of the methods disclosed herein, the subject is a human subject.

The present disclosure also provides a method for modulating NIK activity, comprising exposing NIK to an effective amount of a compound of the disclosure, or a pharmaceutically acceptable salt thereof.

In some embodiments, the disclosure provides a method for inhibiting NIK activity, comprising exposing NIK to an effective amount of a compound of the disclosure, or a pharmaceutically acceptable salt thereof.

### Dosing and Administration

In the methods disclosed herein, an effective amount of at least one compound according to the disclosure is administered to a subject suffering from or diagnosed as having such a disease, disorder, or medical condition. An “effective amount” means an amount or dose sufficient to generally bring about the desired therapeutic or prophylactic benefit in patients in need of such treatment for the designated disease, disorder, or medical condition. For a 70-kg human, an illustrative range for a dosage amount is from about 1 to 1000 mg/day in single or multiple dosage units.

In some embodiments, the dosage amount is about 1 mg to 500 mg of a compound of the disclosure, or a pharmaceutically acceptable salt thereof. In some embodiments, the dosage amount is about 1, 5, 10, 15, 20, 25, 30, 35, 40, 45, 50, 55, 60, 65, 70, 75, 80, 85, 90, 95, or 100 mg of a compound of the disclosure, or pharmaceutically acceptable salt thereof. In some embodiments, the dosage amount is about 100, 105, 110, 115, 120, 125, 130, 135, 140, 145, 150, 155, 160, 165, 170, 175, 180, 185, 190, 195, or 200 mg of a compound of the disclosure, or pharmaceutically acceptable salt thereof. In some embodiments, the dosage amount is about 200, 205, 210, 215, 220, 225, 230, 235, 240, 245, 250, 255, 260, 265, 270, 275, 280, 285, 290, 295, or 300 mg of a compound of the disclosure, or pharmaceutically acceptable salt thereof. In some embodiments, the dosage amount is about 300, 315, 320, 325, 330, 335, 340, 345, 350, 355, 360, 365, 370, 375, 380, 385, 390, 395, or 400 mg of a compound of the disclosure, or pharmaceutically acceptable salt thereof. In some embodiments, the dosage amount is about 400, 405, 410, 415, 420, 425, 430, 435, 440, 445, 450, 455, 460, 465, 470, 475, 480, 485, 490, 495, or 500 mg of a compound of the disclosure, or pharmaceutically acceptable salt thereof

The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present disclosure or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of the present disclosure.

Once improvement of the patient's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

The compounds of the present disclosure, or pharmaceutically acceptable salts thereof, may be formulated into pharmaceutical compositions comprising any known pharmaceutically acceptable carriers. Pharmaceutically acceptable carriers commonly used in pharmaceutical compositions are substances that are non-toxic, biologically tolerable, and otherwise biologically suitable for administration to a subject, such as an inert substance, added to a pharmacological composition or otherwise used as a vehicle or diluent to facilitate administration of an agent and that is compatible therewith. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

Delivery forms of the pharmaceutical compositions containing one or more compounds of the disclosure may be prepared using pharmaceutically acceptable excipients and compounding techniques known or that become available to those of ordinary skill in the art. The compositions may be administered in the presently disclosed methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, subcutaneous injection, topical administration, or oral administration.

For oral administration, the compounds of the present disclosure can be provided in the form of tablets, capsules, or beads, or as a solution, emulsion, or suspension. To prepare the oral compositions, the active agents may be formulated to yield a dosage of, e.g., for a 70-kg human, from about 1 to 1000 mg/day in single or multiple dosage units as an illustrative range.

Oral tablets may include a compound of the disclosure mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Illustrative examples of liquid oral excipients include ethanol, glycerol, water, and the like. Starch, polyvinyl-pyrrolidone (PVP), sodium starch glycolate, microcrystalline cellulose, and alginic acid are examples of disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating. Additional coatings that may be used include coatings that are designed to release the compound or active agent as a function of time, pH or bacterial content.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Soft gelatin capsules may be prepared by mixing the active ingredient with an oil such as peanut oil or olive oil, liquid paraffin, a mixture of mono and di-glycerides of short chain fatty acids, polyethylene glycol 400, or propylene glycol. Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of the present disclosure may also be administered by non-oral routes. For example, compositions may be formulated for rectal administration as a suppository, enema or foam. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the compounds of the disclosure may be mixed with a pharmaceutical carrier. Another mode of administering the compounds of the disclosure may utilize a patch formulation to effect transdermal delivery.

Compounds of the disclosure may alternatively be administered in methods of the present disclosure by inhalation, via the nasal or oral routes, e.g., in a spray formulation also containing a suitable carrier.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the invention as set forth in the claims. Furthermore, the following examples are illustrative, but not limiting, of the compounds, compositions and methods described herein. Other suitable modifications and adaptations known to those skilled in the art are within the scope of the following embodiments. Any and all journal articles, patent applications, issued patents, or other cited references are incorporated by reference in their entirety

### EXAMPLES

The following specific examples are provided to further illustrate embodiments within the scope of the present disclosure.

### Abbreviations

| | |
|---|---|
| Ac | acyl or acetyl |
| ACN or MeCN | acetonitrile |
| AcOH | acetic acid |
| BINAP | 1,1′-binaphthalene]-2,2′-diylbis[diphenylphosphine |
| (BPin)₂ | bis(pinacolato)diboron |
| br | broad |
| BrettPhos | 2-(dicyclohexylphosphino)3,6-dimethoxy-2′,4′,6′- |
| | triisopropyl-1,1′-biphenyl |
| BrettPhos Pd G3 | [(2-di-cyclohexylphosphino-3,6-dimethoxy-2′,4′,6′- |
| | triisopropyl-1,1′-biphenyl)-2-(2′-amino-1,1′- |
| | biphenyl)]palladium(II) methanesulfonate |
| Bu | butyl |
| n-BuLi | n-butyllithium |
| t-BuOH | t-butanol |
| t-BuOK | potassium t-butoxide |
| t-BuONa | sodium t-butoxide |
| Cu(OAc)₂ | copper(II) acetate |
| d | doublet |
| DAC | dynamic axial compression |
| DBU | 1,8-diazabicyclo(5.4.0)undec-7-ene |
| DCM | dichloromethane |
| DEA | diethylamine |
| DIAD | diisopropyl azodicarboxylate |
| DIEA or DIPEA | diisoproylethylamine |
| DMA | dimethylacetamide |
| DMAP | 4-(dimethylamino)pyridine |
| DMF | N,N-dimethylformamide |
| DMSO | dimethyl sulfoxide |
| ESI | electrospray ionization |
| Et | ethyl |
| Et₂NH | diethylamine |
| Et₂O | diethyl ether |
| EtOAc or EA | ethyl acetate |
| EtOH | ethanol |
| FA | Formic acid |
| FCC | flash column chromatography |
| h or hr | hour(s) |
| HATU | 1-[Bis(dimethylamino)methylene]-1H-1,2,3-triazolo[4,5- |
| | b]pyridinium 3-oxid hexafluorophosphate |
| HPLC | high pressure liquid chromatography |
| Hz | Hertz |
| ICl | (2R,3R)-rel-3-isopropylamino-1-(7-methylindan-4- |
| | yloxy)-butan-2-ol hydrochloride |
| IPA or i-PrOH | isopropanol |
| KOAc | potassium acetate |
| Lawesson's reagent | 2,4-Bis(4-methoxyphenyl)-1,3,2,4- |
| | dithiadiphosphetane-2,4-disulfide |
| LCMS | liquid chromatography mass spectrometry |
| LDA | lithium diisopropylamide |
| LiHMDS | lithium bis(trimethylsilyl)amide |
| LiOtBu | lithium t-butoxide |
| mCPBA or m-CPBA | meta-chloroperoxybenzoic acid |
| mmol | millimoles |
| m/z | mass-to-charge ratio |
| M+ | parent molecular ion |
| Me | methyl |
| MeNH₂ | methylamine |
| MeOH | methanol |
| min | minute(s) |
| MS | mass spectrometry |
| MTBE or TBME | tert-butyl methyl ether |
| NaHMDS | sodium bis(trimethylsilyl)amide |
| NaOMe | sodium methoxide |
| NaSO₂Me | sodium methyl sulfate |
| NCS | N-chlorosuccinimide |
| NBS | N-bromosuccinimide |
| NIS | N-iodosuccinimide |
| NMP | N-methyl-2-pyrrolidone |
| NMR | nuclear magnetic resonance |
| nt | not tested |
| Pd₂(dba)₃ | tris(dibenzylideneacetone)dipalladium(0) |
| Pd(t-Bu₃P)₂ | bis(tri-tert-butylphosphine)palladium(0) |
| PdCl₂(dppf) or Pd(dppf)Cl₂ | [1,1- |
| | bis(diphenylphosphino)ferrocene]dichloropalladium(II) |
| Pd(dtbpf)Cl₂ | [1,1-bis(di-tert- |
| | butylphosphino)ferrocene]dichloropalladium(II) |
| Pd(PPh₃)₄ | tetrakis(triphenylphosphine)palladium(0) |
| PdCl₂(PPh₃)₂ or | bis(triphenylphosphine)palladium(II) dichloride |
| Pd(PPh₃)₂Cl₂ | |
| Pd(OAc)₂ | palladium(II) acetate |
| PE or pet ether | petroleum ether |
| PivOH | 2,2-dimethylpropanoic acid |
| RP | reversed-phase |
| rt | room temperature |
| RuPhos Pd G3 | [2′-(Amino-κN)[1,1′-biphenyl]-2-yl-κC][[2′,6′-bis(1- |
| | methylethoxy)[1,1′-biphenyl]-2- |
| | yl]dicyclohexylphosphine-κP](methanesulfonato- |
| | κO)palladium |
| RuPhos Pd G4 | (SP-4-3)-[[2′,6′-Bis(1-methylethoxy)[1,1′-biphenyl]-2- |
| | yl]dicyclohexylphosphine-κP](methanesulfonato-κO)[2′- |
| | (methylamino-κN)[1,1′-biphenyl]-2-yl-κC]palladium |
| sat. | saturated |
| SFC | supercritical fluid chromatography |
| Select-F | selectfluor |
| SEM-Cl | 2-(trimethylsilyl)ethoxymethyl chloride |
| (SnBu₃)₂ | bis(tributyltin) |
| TBAF | tetrabutylammonium fluoride |
| TBSCl | tert-butyldimethylsilyl chloride |
| t-BuOK | potassium tert-butoxide |
| TEA or Et₃N | triethylamine |
| TFA | trifluoroacetic acid |
| THF | tetrahydrofuran |
| TLC | thin layer chromatography |
| TosCl or TsCl | p-toluenesulfonyl chloride |
| TsOH | p-toluenesulfonic acid |
| UV | ultraviolet |
| v/v | volume-to-volume ratio |
| XPhos | 2-dicyclohexylphosphino-2′,4′,6′-triisopropylbiphenyl |
| XPhos Pd G2 | chloro(2-dicyclohexylphosphino-2′,4′,6′-triisopropyl- |
| | 1,1′-biphenyl)[2-(2′-amino-1,1′-biphenyl)]palladium(II) |

In some embodiments, provided herein are processes and intermediates disclosed herein that are useful for preparing a compound of the disclosure or pharmaceutically acceptable salts thereof.

By way of illustration, but not as limitation, compounds of the present disclosure are prepared according to the following general preparation procedures given by Schemes 1-7. One of ordinary skill in the art will recognize that, to obtain the various compounds herein, starting materials may be suitably selected so that the ultimately desired substituents will be carried through the reaction scheme with or without protection as appropriate to yield the desired product. Alternately, in the place of the ultimately desired substituent, a suitable group may be carried through the reaction scheme and replaced, as appropriate, with the desired substituent. Unless otherwise specified, the variables in Schemes 1-7 are as defined above in reference to Formula I.

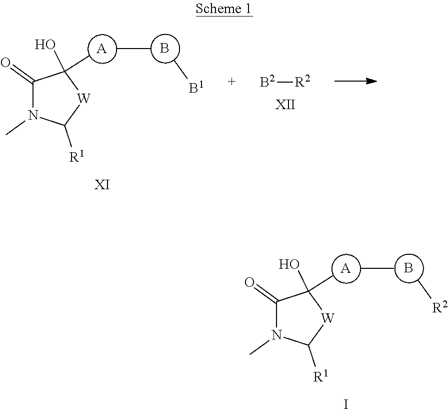

The compounds of Formula I of the present disclosure can be prepared, for example, as shown in Scheme 1. When substituent B¹ in compound XI is a boron-based coupling agent such as a boronic acid or a boronic ester and when substituent B² in compound XII is a halide such as Cl, Br, or I, this coupling is achieved by reaction under Suzuki conditions. One having skill in the art would recognize that coupling under Suzuki conditions also provides a compound of Formula I when B¹ in compound XI is a halide and B² in compound XII is a boron-based coupling agent. Typical Suzuki coupling conditions involve the use of a palladium catalyst, a base, a suitable solvent, and other optional reagents including ligands (e.g., tri-tert-butylphosphine or butyldi(1-adamantanyl)phosphine). Examples of suitable palladium catalysts include, but are not limited to, Pd(t-Bu₃P)₂, Pd(PPh₃)₄, Pd(dtbpf)Cl₂, Pd₂(dba)₃, RuPhos Pd G4, and Pd(dppf)Cl₂. Suitable bases include, but are not limited to, K₃PO₄, KF, Cs₂CO₃, NaHCO₃, Na₂CO₃, and mixtures thereof. Suitable solvents include, but are not limited to, 1,4-dioxane, 1,2-dioxane, DMF, THF, water, and mixtures thereof. The reaction may be heated to a temperature of about 65° C. to about 170° C. for a time period of about 1 to 20 hours, employing microwave or conventional heating to provide the compound of Formula I.

Alternately, Stille coupling conditions can be used to couple compound XI to compound XII when B¹ is a halide such as Cl, Br, or I, and when B² is a stannyl group (e.g., Sn(Me)₃ or Sn(n-Bu)₃). One having skill in the art would recognize that coupling under Stille conditions also provides a compound of Formula I when B¹ is a stannyl group and when B² is a halide. Typical Stille coupling conditions involve the use of a catalyst (usually palladium, but sometimes nickel), a suitable solvent, and other optional reagents including ligands, TEA, or CuI. Examples of suitable catalysts include, but are not limited to Pd(PPh₃)₄ and PdCl₂(PPh₃)₂. Suitable solvents include, but are not limited to, DMF, toluene, and mixtures thereof. The reaction may be heated to a temperature from about 120° C. to about 160° C. for a time period of about 1 to 16 hours, employing microwave or conventional heating to provide the compound of Formula I.

Where a protecting or masking group is present on a compound of Formula XI or XII, a final deprotection step is added, employing conditions known to one skilled in the art, to provide the compound of Formula I. For example, if a methyl or ethyl ester is used to mask a primary amide, it may be unmasked using a reagent such as ammonia in a solvent such as MeOH or EtOH. Alternately, if a nitrile group is used to mask a primary amide, it may be converted to the amide using a reagent such as NaOMe in a solvent such as MeOH. Alternately still, if a carboxylic acid is used to mask a primary amide, it may be converted to the amide using reagents such as NH₄Cl, HATU, and TEA in a solvent such as DMF.

By way of further example, if a tosyl or phthalimide group is used to protect an amino group, it can be removed using a reagent such as ammonia in a solvent such as MeOH or EtOH. Alternately, if a tert-butyloxycarbonyl (Boc) or tetrayhydropyran (THP) group is used to protect an amino group, it can be removed using a reagent such as HCl in a solvent such as dioxane and/or MeOH. Alternately still, if a 2-(trimethylsilyl)ethoxymethyl (SEM) group is used to protect an amino group, it can be removed using a reagent such as TBAF in a solvent such as MeOH. Alternately still, if a para-methoxybenzyl (PMB) group is used to protect an amino group, it can be removed using a reagent such as TFA.

By way of further example, if a nitro group is used to mask a primary amine, it may be converted to the amine using reagents such as porous nickel-aluminum alloy (e.g., Raney Nickel) and hydrazine hydrate in a solvent such as ethanol and/or 1,2-dichloroethane. Alternately, the nitro group may be converted to the anime using reagents such as zinc dust and NH₄Cl in a solvent such as MeOH and/or water.

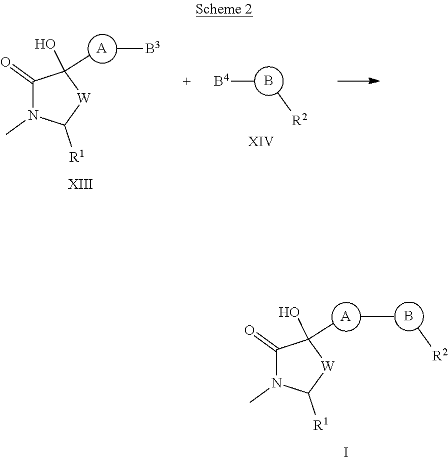

The compounds of Formula I of the present disclosure can be prepared, for example, as shown in Scheme 2. When substituent B³ in compound XIII is a boron-based coupling agent such as a boronic acid or a boronic ester and when substituent B⁴ in compound XIV is a halide such as Cl, Br, or I, this coupling is achieved by reaction under Suzuki conditions. One having skill in the art would recognize that coupling under Suzuki conditions also provides a compound of Formula I when B³ in compound XIII is a halide and B⁴ in compound XIV is a boron-based coupling agent. Typical Suzuki coupling conditions involve the use of a palladium catalyst, a base, a suitable solvent, and other optional reagents including ligands (e.g., tri-tert-butylphosphine or butyldi(1-adamantanyl)phosphine). Examples of suitable palladium catalysts include, but are not limited to, Pd(t-Bu₃P)₂, Pd(PPh₃)₄, Pd(dtbpf)Cl₂, Pd₂(dba)₃, RuPhos Pd G4, and Pd(dppf)Cl₂. Suitable bases include, but are not limited to, K₃PO₄, KF, Cs₂CO₃, NaHCO₃, Na₂CO₃, and mixtures thereof. Suitable solvents include, but are not limited to, 1,4-dioxane, 1,2-dioxane, DMF, THF, water, and mixtures thereof. The reaction may be heated to a temperature of about 65° C. to about 170° C. for a time period of about 1 to 20 hours, employing microwave or conventional heating to provide the compound of Formula I.

Alternately, Stille coupling conditions can be used to couple compound XIII to compound XIV when B³ is a halide such as Cl, Br, or I, and when B⁴ is a stannyl group (e.g., Sn(Me)₃ or Sn(n-Bu)₃). One having skill in the art would recognize that coupling under Stille conditions also provides a compound of Formula I when B³ is a stannyl group and when B⁴ is a halide. Typical Stille coupling conditions involve the use of a catalyst (usually palladium, but sometimes nickel), a suitable solvent, and other optional reagents including ligands, TEA, or CuI. Examples of suitable catalysts include, but are not limited to Pd(PPh₃)₄ and PdCl₂(PPh₃)₂. Suitable solvents include, but are not limited to, DMF, toluene, and mixtures thereof. The reaction may be heated to a temperature from about 120° C. to about 160° C. for a time period of about 1 to 16 hours, employing microwave or conventional heating to provide the compound of Formula I.

Where a protecting or masking group is present on a compound of Formula XIII or XIV, a final deprotection step is added, employing conditions known to one skilled in the art, to provide the compound of Formula I. For example, if a 2-(trimethylsilyl)ethoxymethyl (SEM) group is used to protect an amino group, it can be removed using a reagent such as TBAF in a solvent such as MeOH.

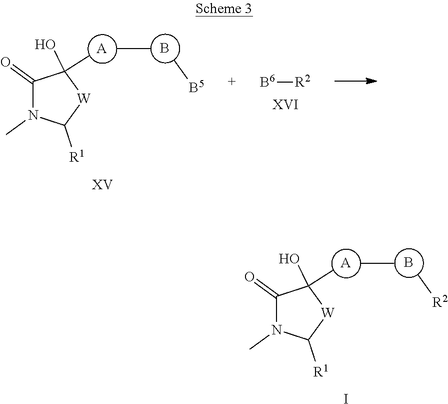

The compounds of Formula I of the present disclosure can be prepared, for example, as shown in Scheme 3 when the bond between B and R² is between a carbon atom and a nitrogen atom. In some embodiments, substituent B⁵ is a halide, and substituent B⁶ is a hydrogen atom covalently bonded to a nitrogen atom of R². Alternately, substituent B⁵ is a hydrogen atom covalently bonded to a nitrogen atom of B, and B⁶ is a halide. Compounds XV and XVI may be combined with a suitable catalyst (e.g., a palladium or copper catalyst), a suitable base, a suitable solvent, and other optional reagents including ligands. Suitable catalysts include, but are not limited to, Pd₂(dba)₃ and CuI. Suitable bases include, but are not limited to, t-BuONa and K₃PO₄. Suitable solvents include, but are not limited to, DMF and toluene. Suitable ligands include, but are not limited to BINAP and trans-N,N′-dimethylcyclohexane-1,2-diamine. The reaction may be heated to a temperature from about 85° C. to about 100° C. for a time period of about 16 hours to provide the compound of Formula I.

Alternately, compounds XV and XVI may be combined with a suitable base and a suitable solvent. Suitable bases include, but are note limited to, Cs₂CO₃ and TEA. Suitable solvents include, but are not limited to DMF and DMA. The reaction may be heated to a temperature from about 100° C. to about 140° C. for a time period of about 3 to 24 hours, employing microwave or conventional heating to provide the compound of Formula I.

Where a protecting or masking group is present on a compound of Formula XV or XVI, a final deprotection step is added, employing conditions known to one skilled in the art, to provide the compound of Formula I. For example, if a methyl or ethyl ester is used to mask a primary amide, it may be unmasked using a reagent such as ammonia in a solvent such as MeOH or EtOH.

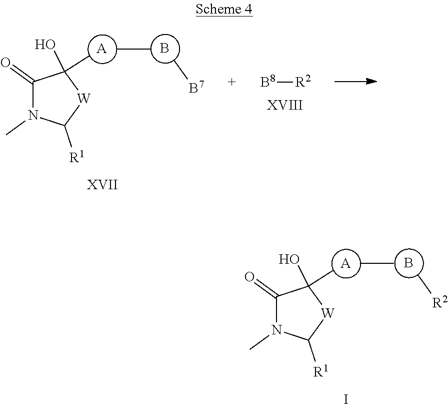

The compounds of Formula I of the present disclosure can be prepared, for example, as shown in Scheme 4. In some embodiments, substituent B⁷ is a boron-based coupling agent such as a boronic acid or a boronic ester, and substituent B⁸ is a hydrogen atom covalently bonded to a nitrogen atom of R². Compounds XVII and XVIII may be combined with a suitable catalyst (e.g., Cu(OAc)₂), a suitable base (e.g., pyridine), a suitable solvent, and other optional reagents (e.g., molecular seives). Suitable solvents include, but are not limited to, DMF and chloroform. The reaction may be heated to a temperature from about 40° C. to about 120° C. for a time period of about 3 to 72 hours to provide the compound of Formula I.

Where a protecting or masking group is present on a compound of Formula XVII or XVIII, a final deprotection step is added, employing conditions known to one skilled in the art, to provide the compound of Formula I. For example, if a methyl or ethyl ester is used to mask a primary amide, it may be unmasked using a reagent such as ammonia in a solvent such as MeOH or EtOH.

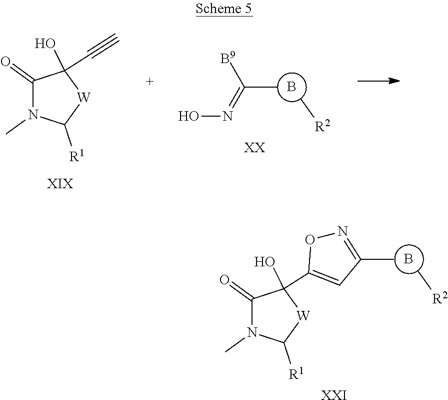

The compounds of Formula XXI of the present disclosure can be prepared, for example, as shown in Scheme 5. Substituent B⁹ in compound XX is, for example, a halide such as Cl. Compounds XIX and XX may be combined with a suitable base (e.g., TEA) and a suitable solvent (e.g., DCM). The reaction may be heated to a temperature from about 25° C. to about 40° C. for a time period of about 3 to 16 hours to provide the compound of Formula XXI.

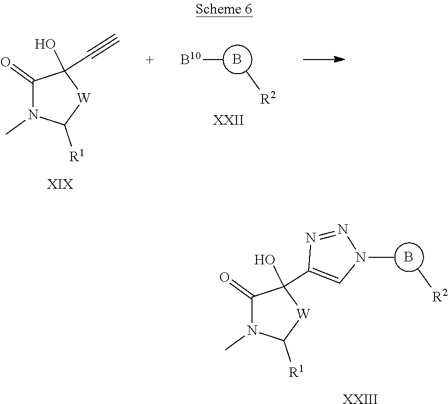

The compounds of Formula XXIII of the present disclosure can be prepared, for example, as shown in Scheme 6. Substituent B¹⁰ in compound XXII is, for example, a halide such as Cl. Compounds XIX and XXII may be combined with a copper catalyst (e.g., CuI), a ligand (e.g., trans-N,N′-dimethylcyclohexane-1,2-diamine), and NaN₃ in a solvent (e.g., DMSO and/or water). An additive, such as sodium ascorbate may also be included. The reaction may be allowed to progress at room temperature for about 16 hours to provide the compound of Formula XXIII.

Where a protecting or masking group is present on a compound of Formula XXII, a final deprotection step is added, employing conditions known to one skilled in the art, to provide the compound of Formula XXIII. For example, if a methyl or ethyl ester is used to mask a primary amide, it may be unmasked using a reagent such as ammonia in a solvent such as MeOH or EtOH.

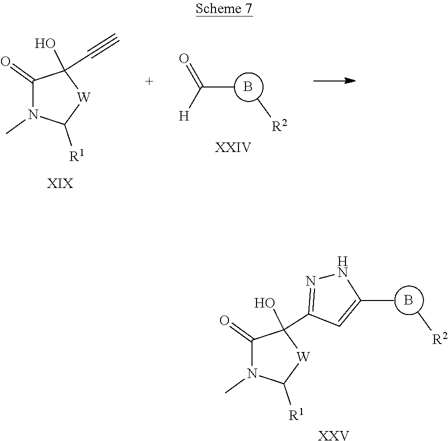

The compounds of Formula XXV of the present disclosure can be prepared, for example, as shown in Scheme 7. Compound XXIV may be combined with p-toluenesulfonyl hydrazide and a suitable solvent (e.g., ACN), and the reaction mixture may be allowed to progress at room temperature for about 3 hours. A suitable base (e.g., NaOH) and the compound of Formula XIX may then be added to the reaction mixture. The reaction may subsequently be heated to a temperature of about 50° C. for about 16 hours to provide the compound of Formula XXV.

In obtaining the compounds described in the examples below and the corresponding analytical data, the following experimental and analytical protocols were followed unless otherwise indicated.

Unless otherwise specified, reaction solutions were stirred at room temperature under a N_{2(g)} or Ar_{(g)} atmosphere. When solutions were “concentrated to dryness”, they were concentrated using a rotary evaporator under reduced pressure. When solutions were dried, they are typically dried over a drying agent such as MgSO₄ or Na₂SO₄. Normal phase flash column chromatography (FCC) was performed on silica gel with prepackaged silica gel columns, such as RediSep®, using ethyl acetate (EtOAc)/hexanes, CH₂Cl₂/MeOH, or CH₂Cl₂/10% 2N NH₃ in MeOH, as eluent, unless otherwise indicated.

Thin-layer chromatography was performed using silica gel plates, such as Merck silica gel 60 F₂₅₄ 2.5 cm×7.5 cm 250 mm or 5.0 cm×10.0 cm 250 μm pre-coated silica gel plates. Preparative thin-layer chromatography was performed using silica gel plates such as EM Science silica gel 60 F₂₅₄ 20 cm×20 cm 0.5 mm pre-coated plates with a 20 cm×4 cm concentrating zone. Microwave reactions were carried out in a microwave reactor, such as a CEM Discover^{â}, a Biotage Initiator™ or Optimizer™ microwave, at specified temperatures. Mass spectra were obtained on a mass spectrometer, such as Agilent series 1100 MSD using electrospray ionization (ESI) in positive mode unless otherwise indicated. Calculated mass corresponds to the exact mass. NMR spectra were obtained on an NMR spectrometer, such as a Bruker model DPX400 (400 MHz), DPX500 (500 MHz), DRX600 (600 MHz) spectrometer. The format of the ¹H NMR data below is as follows: Chemical shift in ppm down field of the tetramethylsilane reference (multiplicity, coupling constant J in Hz, integration).

Whenever a yield is given as a percentage, such yield refers to a mass of the entity for which the yield is given with respect to the maximum amount of the same entity that could be obtained under the particular stoichiometric conditions. Reagent concentrations that are given as percentages refer to mass ratios, unless indicated differently. Whether expressly indicated or not, yields given in the following examples are computed with respect to the dried form of the compound for which any such yield is given.

Chemical names were generated using ChemDraw Ultra 17.1 (CambridgeSoft Corp., Cambridge, MA) or OEMetaChem V1.4.0.4 (Open Eye).

### Intermediate 1: (R)-3-Ethynyl-3-hydroxy-1-methylpyrrolidin-2-one

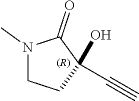

Step A: tert-Butyl 3-(methylamino)propanoate. A 2 L round-bottomed flask equipped with an overhead stirrer was charged with methylamine (500 mL, 3.48 mol, 30 wt % in EtOH) and EtOH (500 mL) followed by dropwise addition of tert-butyl acrylate (100 g, 0.78 mol) over 3 h at 20-25° C. The resultant mixture was stirred at rt for 3 h and then concentrated to dryness to give tert-butyl 3-(methylamino)propanoate (124 g) as a colorless oil. MS (ESI): Mass calcd. for C₈H₁₇NO₂, 159.1; m/z found, 160.2 [M+H]⁺. ¹H NMR (400 MHz, CDCl₃) δ 2.79 (t, J=6.5 Hz, 2H), 2.43 (s, 3H), 2.41 (t, J=6.5 Hz, 2H), 1.44 (s, 9H).

Step B: tert-Butyl 4-hydroxy-1-methyl-5-oxo-2,5-dihydro-1H-pyrrole-3-carboxylate. A 50 L glass-lined reactor equipped with an overhead stirrer was charged with tert-butyl 3-(methylamino)propanoate (900 g, 5.65 mol), diethyl oxalate (827 g, 5.65 mol) and THF (18 L). The resultant mixture was warmed to 50-55° C. followed by addition of t-BuOK (633 g, 5.65 mol) batch-wise. After stirring for 1 h, the mixture was cooled to 20° C., concentrated to dryness, and water (5.00 L) was added which resulted in the formation of a suspension. The pH was adjusted to 1-2 with aqueous HCl and the resultant mixture was stirred at 20-25° C. for 1 h followed by filtration and drying to give tert-butyl 4-hydroxy-1-methyl-5-oxo-2,5-dihydro-1H-pyrrole-3-carboxylate (940 g, 78%) as an off-white solid. ¹H NMR (300 MHz, CDCl₃) δ 8.99 (s, 1H), 3.94 (s, 2H), 3.10 (s, 3H), 1.56 (s, 9H).

Step C: 4-Hydroxy-1-methyl-5-oxo-2,5-dihydro-1H-pyrrole-3-carboxylic acid. A 5 L round-bottomed flask equipped with an overhead stirrer was charged with tert-butyl 4-hydroxy-1-methyl-5-oxo-2,5-dihydro-1H-pyrrole-3-carboxylate (500 g, 2.34 mol) and TFA (2.00 L). The resultant mixture was stirred at 20-25° C. for 3 h and then concentrated to dryness. To the residue was added acetonitrile (1.50 L) with stirring at 20-25° C. for 1 h. The product was isolated by filtration and dried to give 4-hydroxy-1-methyl-5-oxo-2,5-dihydro-1H-pyrrole-3-carboxylic acid (357 g, 97%) as an off-white solid. ¹H NMR (300 MHz, CD₃OD) δ 4.04-3.98 (m, 2H), 3.08 (s, 3H).

Step D: 1-Methylpyrrolidine-2,3-dione. A 20 L round-bottomed flask equipped with an overhead stirrer was charged with 4-hydroxy-1-methyl-5-oxo-2,5-dihydro-1H-pyrrole-3-carboxylic acid (1000 g, 6.4 mol) and THF (15 L). The resultant mixture was heated to 65° C. After 4 h, the mixture was concentrated to dryness to give 1-methylpyrrolidine-2,3-dione (712 g, 99%) as a yellow solid. ¹H NMR (400 MHz, CDCl₃) δ 3.70 (t, J=5.7 Hz, 2H), 3.13 (s, 3H), 2.72 (t, J=5.7 Hz, 2H).

Step E: (R,S)-3-Ethynyl-3-hydroxy-1-methylpyrrolidin-2-one. A 10 L round-bottomed flask equipped with an overhead stirrer was charged with ethynylmagnesiumbromide (3.50 L, 0.5 M in THF). The flask was purged with nitrogen and cooled to −10° C. before adding 1-methylpyrrolidine-2,3-dione (120 g, 1.06 mol) over the course of 20 min. The resultant mixture was warmed to 20-25° C. and stirred for 16 h. The resulting mixture was quenched with aqueous NH₄Cl solution (120 g in 360 mL H₂O) followed by dilution with DCM (3.50 L). After being slurried for 1 h, the suspension was filtered, and the filtrate was dried over anhydrous Na₂SO₄ (500 g) and treated with activated charcoal (24 g). The activated charcoal was removed by filtration and the filtrate was concentrated under vacuum to dryness. The residue was slurried in MTBE (360 mL) at 20-25° C. for 1 h. The product was isolated by filtration followed by drying to give (R,S)-3-ethynyl-3-hydroxy-1-methylpyrrolidin-2-one (81 g, 55%) as a yellow solid. MS (ESI): Mass calcd. for C₇H₉NO₂, 139.1; m/z found, 140.1 [M+H]⁺. ¹H NMR (300 MHz, CD₃OD) δ 3.40 (dd, J=7.7, 5.3 Hz, 2H), 3.03 (s, 1H), 2.88 (s, 3H), 2.52-2.41 (m, 1H), 2.21 (dt, J=12.7, 7.7 Hz, 1H).

Step F: (R)-3-Ethynyl-3-hydroxy-1-methylpyrrolidin-2-one and (S)-3-Ethynyl-3-hydroxy-1-methylpyrrolidin-2-one. The enantiomers of (R,S)-3-ethynyl-3-hydroxy-1-methylpyrrolidin-2-one were separated by chiral preparative SFC using an SFC column, such as a CHIRALPAK® AS-H 5 μm, 5×25 cm column, mobile phase (80% CO₂, 20% IPA (0.1% DEA). Detection, UV at 25° C.=220-254 nM) to yield (R)-3-ethynyl-3-hydroxy-1-methylpyrrolidin-2-one (40%) and (S)-3-ethynyl-3-hydroxy-1-methylpyrrolidin-2-one (Intermediate 2, 40%). Data for (R)-3-ethynyl-3-hydroxy-1-methylpyrrolidin-2-one: MS (ESI): Mass calcd. for C₇H₉NO₂, 139.1; m/z found, 140.1 [M+H]⁺. ¹H NMR (300 MHz, CD₃OD) δ 3.40 (dd, J=7.7, 5.3 Hz, 2H), 3.03 (s, 1H), 2.88 (s, 3H), 2.52-2.41 (m, 1H), 2.21 (dt, J=12.7, 7.7 Hz, 1H). [α]²⁰_{D}=−100.1 (c=1.01 in MeOH).

### Intermediate 2: (S)-3-Ethynyl-3-hydroxy-1-methylpyrrolidin-2-one

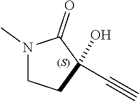

The chiral separation described in Intermediate 1, Step F also provided (S)-3-ethynyl-3-hydroxy-1-methylpyrrolidin-2-one (40%). MS (ESI): Mass calcd. for C₇H₉NO₂, 139.1; m/z found, 140.1 [M+H]⁺. ¹H NMR (300 MHz, CD₃OD) δ 3.40 (dd, J=7.7, 5.3 Hz, 2H), 3.03 (s, 1H), 2.88 (s, 3H), 2.52-2.41 (m, 1H), 2.21 (dt, J=12.7, 7.7 Hz, 1H). [α]²⁰_{D}=+90.5 (c=1.19 in MeOH).

### Intermediate 3: (R)-3-(3-(3-Bromophenyl)isoxazol-5-yl)-3-hydroxy-1-methylpyrrolidin-2-one

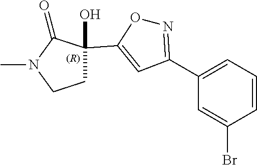

Step A: 3-Bromobenzaldehyde oxime. To a solution of 3-bromobenzaldehyde (3.0 g, 16 mmol) in EtOH (40 mL) was added NH₂OH (50% in water, 1.1 mL, 19 mmol). The reaction was stirred at 25° C. for 4 h. The reaction mixture was then concentrated under reduced pressure to afford (3.2 g, 98%) of 3-bromobenzaldehyde oxime as a white solid. MS (ESI): Mass calcd. for C₇H₆BrNO, 199.0; m/z found, 200.1 [M+H]⁺.

Step B: 3-Bromo-N-hydroxybenzimidoyl chloride. To a solution of 3-bromobenzaldehyde oxime (3.20 g, 16.0 mmol) in anhydrous DCM (20 mL) was added N-chlorosuccinimide (2.56 g, 19.2 mmol), portion wise. The reaction mixture was stirred at 25° C. for 2 h and filtered. The filtrate was concentrated to afford 3-bromo-N-hydroxybenzimidoyl chloride (3.0 g, 95%) as a yellow solid. MS (ESI): Mass calcd. For C₇H₅BrClNO, 234.5; m/z found, 235.2 [M+H]⁺.

Step C: (R)-3-(3-(3-Bromophenyl)isoxazol-5-yl)-3-hydroxy-1-methylpyrrolidin-2-one. To a solution of 3-bromo-N-hydroxybenzimidoyl chloride (1.50 g, 3.20 mmol) in DCM (20 mL) was added (R)-3-ethynyl-3-hydroxy-1-methylpyrrolidin-2-one (Intermediate 1, 4.45 g, 3.20 mmol) followed by TEA (1.24 mL, 9.60 mmol). The reaction mixture was stirred at 25° C. for 16 h, diluted with DCM (30 mL), washed with brine (20 mL), dried over anhydrous Na₂SO₄, filtered and concentrated under reduced pressure. The residue was purified by preparative TLC (petroleum ether/ethyl acetate=1/1) to afford (R)-3-(3-(3-bromophenyl)isoxazol-5-yl)-3-hydroxy-1-methylpyrrolidin-2-one (0.6 g, 55%) as a yellow oil. MS (ESI): Mass calcd. for C₁₄H₁₃BrN₂O₃, 336.0; m/z found, 337.1 [M+H]⁺.

### Intermediate 4: (R)-3-Hydroxy-1-methyl-3-(3-(3-(4,4,5,5-tetramethyl-1,3,2-dioxaborolan-2-yl)phenyl)isoxazol-5-yl)pyrrolidin-2-one

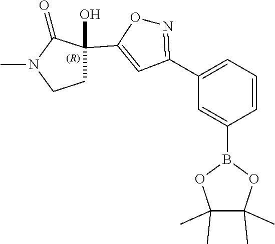

A flask containing (R)-3-(3-(3-bromophenyl)isoxazol-5-yl)-3-hydroxy-1-methylpyrrolidin-2-one (Intermediate 3, 0.60 g, 1.8 mmol) in 1,4-dioxane (10 mL) was charged with bis(pinacolato)diboron (0.90 g, 3.56 mmol), Pd(dppf)Cl₂CH₂Cl₂ (130 mg, 0.180 mmol) and KOAc (540 mg, 5.34 mmol). The reaction mixture was heated at 85° C. for 16 h under an Ar atmosphere. The mixture was concentrated and the residue was purified by preparative TLC (DCM/MeOH=20/1) to afford (R)-3-hydroxy-1-methyl-3-(3-(3-(4,4,5,5-tetramethyl-1,3,2-dioxaborolan-2-yl)phenyl)isoxazol-5-yl)pyrrolidin-2-one (0.62 g, 77%) as a brown solid. MS (ESI): Mass calcd. for C₂₀H₂₅BN₂O₅, 384.2; m/z found, 385.2 [M+H]⁺.

### Intermediate 5: (R)-3-Hydroxy-1-methyl-3-(5-(3-(4,4,5,5-tetramethyl-1,3,2-dioxaborolan-2-yl)phenyl)isoxazol-3-yl)pyrrolidin-2-one

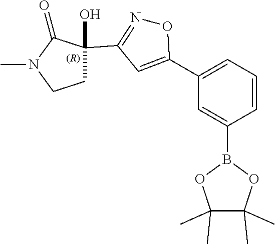

Step A: (R)-3-(3-(3-Bromophenyl)-3-oxoprop-1-ynyl)-3-hydroxy-1-methylpyrrolidin-2-one. A solution of (R)-3-ethynyl-3-hydroxy-1-methylpyrrolidin-2-one (Intermediate 1, 3.00 g, 21.6 mmol) in THF (150 mL) was cooled to −20° C. and PdCl₂(PPh₃)₂ (0.61 g, 0.86 mmol), CuI (0.21 g, 1.08 mmol) and TEA (4.47 mL, 34.1 mmol) were added. A solution of 3-bromobenzoyl chloride (4.73 g, 21.6 mmol) in THF (10 mL) was added dropwise. The reaction mixture was slowly warmed to 0° C., stirred for 2 h, diluted with water (80 mL) and extracted with EtOAc (3×60 mL). The combined organic solvent extracts were washed with brine (60 mL), dried over Na₂SO₄, and filtered. The filtrate was concentrated and purified by FCC (DCM/MeOH=100/1) to afford (R)-3-(3-(3-bromophenyl)-3-oxoprop-1-ynyl)-3-hydroxy-1-methylpyrrolidin-2-one (1.6 g, 23%) as a yellow solid. MS (ESI): Mass calcd. for C₁₄H₁₂BrNO₃, 321.0; m/z found, 322.0 [M+H]⁺.

Step B: (R)-3-(5-(3-Bromophenyl)isoxazol-3-yl)-3-hydroxy-1-methylpyrrolidin-2-one. To a solution of (R)-3-(3-(3-bromophenyl)-3-oxoprop-1-ynyl)-3-hydroxy-1-methylpyrrolidin-2-one (1.60 g, 4.37 mmol) in DCM (100 mL) was added NaN₃ (1.42 g, 21.8 mmol). The mixture was cooled to 0° C. and AcOH (1.31 g, 21.85 mmol) was added dropwise, followed by TEA (1.21 mL, 0.87 mmol). The reaction mixture was stirred at rt for 2 days. Saturated aqueous NaHCO₃ solution (100 mL) was added and the mixture was stirred for 10 min. The organic solvent portion was separated, and the aqueous layer was extracted with DCM (2×50 mL). The combined organic solvent extracts were washed with brine (2×100 mL), dried over Na₂SO₄ and filtered. The filtrate was concentrated by blowing a continuous stream of N₂ over the filtrate and the residue was purified by FCC (DCM/MeOH=50/1) to afford (R)-3-(5-(3-bromophenyl)isoxazol-3-yl)-3-hydroxy-1-methylpyrrolidin-2-one (0.6 g, 41%) as a grey oil. MS (ESI): Mass calcd. for C₁₄H₁₃BrN₂O₃, 336.0; m/z found, 337.0 [M+H]⁺.

Step C: (R)-3-Hydroxy-1-methyl-3-(5-(3-(4,4,5,5-tetramethyl-1,3,2-dioxaborolan-2-yl)phenyl)isoxazol-3-yl)pyrrolidin-2-one. A flask containing (R)-3-(5-(3-bromophenyl)isoxazol-3-yl)-3-hydroxy-1-methylpyrrolidin-2-one (85 mg, 0.252 mmol) in 1,4-dioxane (5 mL) was charged with bis(pinacolato)diboron (128.0 mg, 0.50 mmol), Pd(dppf)Cl₂·CH₂Cl₂ (18.4 mg, 0.025 mmol) and KOAc (74.2 mg, 0.76 mmol). The mixture was heated at 65° C. for 4 h under Ar. The reaction mixture was filtered and concentrated to afford (R)-3-hydroxy-1-methyl-3-(5-(3-(4,4,5,5-tetramethyl-1,3,2-dioxaborolan-2-yl)phenyl)isoxazol-3-yl)pyrrolidin-2-one (0.10 g, 87%) as a brown oil which was used in the next step without further purification. MS (ESI): Mass calcd. for C₂₀H₂₅BN₂O₅, 384.2; m/z found, 385.2 [M+H]⁺.

### Intermediate 6: (R,S)-3-(5-(3-Bromophenyl)isoxazol-3-yl)-3-hydroxy-1-methylpyrrolidin-2-one

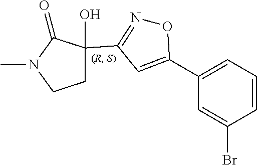 [SMILES: CN1CCC(O)(c2cc(-c3cccc(Br)c3)on2)C1=O]

(R,S)-3-(5-(3-Bromophenyl)isoxazol-3-yl)-3-hydroxy-1-methylpyrrolidin-2-one (1.4 g) was prepared using analogous conditions to those described in the preparation of Intermediate 5 ((R)-3-hydroxy-1-methyl-3-(5-(3-(4,4,5,5-tetramethyl-1,3,2-dioxaborolan-2-yl)phenyl)isoxazol-3-yl)pyrrolidin-2-one) using (R,S)-3-ethynyl-3-hydroxy-1-methylpyrrolidin-2-one (prepared in Intermediate 1, step E) instead of (R)-3-ethynyl-3-hydroxy-1-methylpyrrolidin-2-one. MS (ESI): Mass calcd. for C₁₄H₁₃BrN₂O₃, 336.0; m/z found, 337.0 [M+H]⁺.

### Intermediate 7: (R)-3-(3-(2-Bromopyridin-4-yl)isoxazol-5-yl)-3-hydroxy-1-methylpyrrolidin-2-one

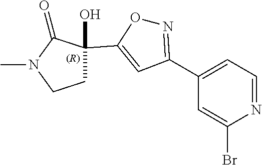

Step A: 2-Bromoisonicotinaldehyde oxime. To a solution of 2-bromoisonicotinaldehyde (250 mg, 1.3 mmol) in EtOH (10 mL) was added hydroxylamine (0.095 mL, 50% in water, 1.6 mmol). The reaction mixture was stirred at rt for 16 h and concentrated to dryness to afford 2-bromoisonicotinaldehyde oxime (240 mg, 89%) as a colorless solid. MS (ESI): Mass calcd. for C₆H₅BrN₂O, 200.0; m/z found, 201.1 [M+H]⁺.

Step B: 2-Bromo-N-hydroxyisonicotinimidoyl chloride. To a solution of 2-bromoisonicotinaldehyde oxime (140 mg, 0.60 mmol) in DCM (5 mL) was added N-chlorosuccinimide (120 mg, 0.90 mmol) portion-wise at rt. The reaction mixture was stirred for 2 h at rt, then DMF (1 mL) was added, and the reaction mixture was stirred for an additional 2 h. The mixture was diluted with EtOAc (20 mL) and washed with brine (3×20 mL). The organic extracts were dried over Na₂SO₄, filtered and concentrated to dryness to afford 2-bromo-N-hydroxyisonicotinimidoyl chloride (140 mg, 80%) as a white solid. MS (ESI): Mass calcd. for C₆H₄BrClN₂O, 235.5; m/z found, 199.0 [M−HCl+1]⁺.

Step C: (R)-3-(3-(2-Bromopyridin-4-yl)isoxazol-5-yl)-3-hydroxy-1-methylpyrrolidin-2-one. To a solution of bromo-N-hydroxyisonicotinimidoyl chloride (2.80 g, 11.9 mmol), and (R)-3-ethynyl-3-hydroxy-1-methylpyrrolidin-2-one (Intermediate 1, 1.64 g, 11.9 mmol) in t-BuOH:H₂O (10 mL 1:1) was added a solution of sodium ascorbate (0.24 g, 1.2 mmol) in H₂O (1 mL). A solution of CuSO₄·5H₂O (0.89 g, 3.6 mmol) in H₂O (2 mL) was then added followed by the addition of KHCO₃ (3.57 g, 1.19 mmol). The reaction mixture was stirred for 16 h at rt, diluted with water (20 mL) and extracted with EtOAc (3×20 mL). The combined organic solvent extracts were dried, filtered, concentrated to dryness and purified by preparative TLC (DCM/MeOH=20/1) to afford (R)-3-(3-(2-bromopyridin-4-yl)isoxazol-5-yl)-3-hydroxy-1-methylpyrrolidin-2-one (1.8 g, 45%) as a yellow solid. MS (ESI): Mass calcd. for C₁₃H₁₂BrN₃O₃, 337.0, m/z found, 338.3 [M+H]⁺.

### Intermediate 8: (R)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one

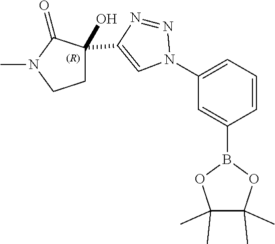

Step A: (R)-3-(1-(3-Bromophenyl)-1H-1,2,3-triazol-4-yl)-3-hydroxy-1-methylpyrrolidin-2-one. To a suspension of 1-bromo-3-iodobenzene (281 mg, 0.99 mmol) in DMSO (2 mL) was added (R)-3-ethynyl-3-hydroxy-1-methylpyrrolidin-2-one (Intermediate 1, 138 mg, 0.993 mmol), sodium azide (71 mg, 1.1 mmol), (1S,2S)-N1,N2-dimethylcyclohexane-1,2-diamine (28 mg, 0.20 mmol) and copper(I) iodide (38 mg, 0.20 mmol), successively. A solution of sodium ascorbate (39 mg, 0.20 mmol) in water (0.5 mL) was slowly added and the reaction mixture was stirred at rt for 16 h under an Ar atmosphere. The mixture was diluted with EtOAc (20 mL), the organic phase was washed with brine (3×15 mL), dried over Na₂SO₄, filtered and concentrated under reduced pressure. The residue was purified by FCC (DCM\MeOH=20/1) to afford (R)-3-(1-(3-bromophenyl)-1H-1,2,3-triazol-4-yl)-3-hydroxy-1-methylpyrrolidin-2-one (200 mg, 76%) as a yellow solid. MS (ESI): Mass calcd. for C₁₃H₁₃BrN₄O₂, 336.0; m/z found, 337.0 [M+H].

Step B: (R)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one. To a solution of (R)-3-(1-(3-bromophenyl)-1H-1,2,3-triazol-4-yl)-3-hydroxy-1-methylpyrrolidin-2-one (200 mg, 0.593 mmol) in dioxane (10 mL) was added bis(pinacolato)diboron (301 mg, 1.19 mmol), followed by [1,1′-bis(diphenylphosphino)ferrocene]dichloropalladium(II) (45 mg, 0.059 mmol) and KOAc (175 mg, 1.78 mmol). The reaction mixture was heated at 65° C. for 2 h under an Ar atmosphere. The reaction mixture was then filtered and concentrated under reduced pressure. The residue was purified by FCC (DCM/MeOH=20/1) to afford (R)-3-hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (260 mg, 68%) as a yellow solid. MS (ESI): Mass calcd. for C₁₉H₂₅BN₄O₄, 384.2; m/z found, 385.2 [M+H]⁺.

### Intermediate 9: (R,S)-3-Hydroxy-1-methyl-3-(1-(3-(4,4,5,5-tetramethyl-1,3,2-dioxaborolan-2-yl)phenyl)-1H-imidazol-4-yl)pyrrolidin-2-one

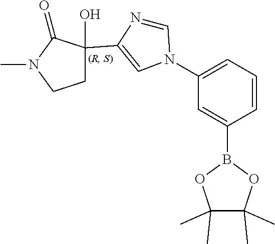

Step A: 1-(3-Bromophenyl)-4-iodo-1H-imidazole. A mixture of 4-iodoimidazole (3.68 g, 19.0 mmol) in THF (100 mL), 3-bromophenylboronic acid (7.6 g, 38 mmol), di-p-hydroxo-bis[(N,N,N′,N′-tetramethylethylenediamine)copper(II)] chloride (9.7 g, 20.869 mmol) and molecular sieves (250 mg) was stirred at 30° C. under an 02 atmosphere for 16 h. The reaction mixture was then filtered through diatomaceous earth, washed with EtOAc (200 mL) and concentrated under reduced pressure. The residue was purified by FCC (petroleum ether/EA=2/1) to afford 1-(3-bromophenyl)-4-iodo-1H-imidazole (3.2 g, 48%) as a brown solid. MS (ESI): Mass calcd. for C₉H₆BrIN₂, 347.9; m/z found, 349.0 [M+H]⁺.

Step B: 3-(1-(3-Bromophenyl)-1H-imidazol-4-yl)-3-hydroxy-1-methylpyrrolidin-2-one. A solution of isopropylmagnesium chloride-lithium chloride (3.6 mL, 1.3 M in THF) was added dropwise to a solution of 1-(3-bromophenyl)-4-iodo-1H-imidazole (1.35 g, 3.87 mmol) in THF (30 mL) that had been cooled to 0° C. After one hour, a solution of 1-methylpyrrolidine-2,3-dione (0.87 g, 7.74 mmol) in THF (10 mL) was added and the mixture was stirred at 0° C. for 2 h. The reaction mixture was allowed to warm to rt and stirred overnight. The reaction mixture was then cooled to 0° C., quenched with ice water (6 mL) and MeOH (10 mL) and concentrated under reduced pressure. The residue was purified using preparative TLC (DCM/MeOH=15/1) to afford 3-(1-(3-bromophenyl)-1H-imidazol-4-yl)-3-hydroxy-1-methylpyrrolidin-2-one (0.29 mg, 22%) as a brown solid. MS (ESI): Mass calcd. for C₁₄H₁₄BrN₃O₂, 336.2; m/z found, 336.0 [M+H]⁺.

Step C: 3-Hydroxy-1-methyl-3-(1-(3-(4,4,5,5-tetramethyl-1,3,2-dioxaborolan-2-yl)phenyl)-1H-imidazol-4-yl)pyrrolidin-2-one. A mixture of bis(pinacolato)diboron (242 mg, 0.95 mmol), 3-(1-(3-bromophenyl)-1H-imidazol-4-yl)-3-hydroxy-1-methylpyrrolidin-2-one (160 mg, 0.48 mmol), Pd(dppf)Cl₂CH₂Cl₂ (36.3 mg, 0.048 mmol) and KOAc (140.1 mg, 1.428 mmol) in dioxane (10 mL) was heated at 80° C. for 16 h under an Ar atmosphere. The reaction mixture was used directly in subsequent reactions. MS (ESI): Mass calcd. for C₂₀H₂₆BN₃O₄, 383.3; m/z found, 384.0 [M+H]⁺.

### Intermediate 10: (R)-(3-(5-(3-Hydroxy-1-methyl-2-oxopyrrolidin-3-yl)isoxazol-3-yl)phenylboronic acid

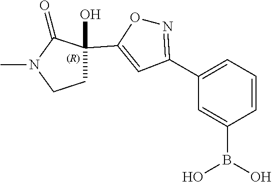

To a suspension of (R)-3-(3-(3-bromophenyl)isoxazol-5-yl)-3-hydroxy-1-methylpyrrolidin-2-one (Intermediate 3, 500 mg, 1.5 mmol) in MeOH (15 mL) was added tetrakis(dimethylaminio)diboron (881 mg, 4.45 mmol) followed by chloro(2-dicyclohexylphosphino-2′,4′,6′-triisopropyl-1,1′-biphenyl)[2-(2′-amino-1,1′-biphenyl)]palladium(II) (43.4 mg, 59.3 mmol) and the reaction was heated at 60° C. for 16 h under an argon atmosphere. The mixture was concentrated to dryness and purified by FCC (8% MeOH/DCM) to afford (R)-(3-(5-(3-hydroxy-1-methyl-2-oxopyrrolidin-3-yl)isoxazol-3-yl)phenyl)boronic acid (160 mg, 36%) as a yellow solid. MS (ESI): Mass calcd. for C₁₄H₁₅BN₂O₅, 302.11; m/z found, 303.2 [M+H]⁺.

### Intermediate 11: 5-Chloro-3-nitro-1H-pyrazolo[4,3-b]pyridine

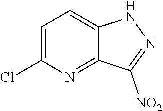 [SMILES: O=[N+]([O-])c1n[nH]c2ccc(Cl)nc12]

To a flask containing 5-chloro-1H-pyrazolo[4,3-b]pyridine (5.0 g, 32.56 mmol) was added H₂SO₄ (10 mL), followed by KNO₃ (8.2 g, 81.397 mmol) at 0° C. The mixture was stirred at rt for 3 h and then heated at 115° C. for 8 h. The reaction mixture was then poured into ice water and the pH was adjusted to pH 9 using aqueous NH₄OH. A precipitate formed and was collected by filtration. The filter cake was dried to afford 5-chloro-3-nitro-1H-pyrazolo[4,3-b]pyridine (5.2 g, 80%) as a yellow solid. MS (ESI): calcd. for C₆H₃ClN₄O₂, 198.6; m/z found, 199.0 [M+H]⁺.

### Intermediate 12: 5-Chloro-1-methyl-3-nitro-1H-pyrazolo[4,3-b]pyridine

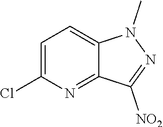

To a solution of 5-chloro-3-nitro-1H-pyrazolo[4,3-b]pyridine (Intermediate 11, 500 mg, 2.5 mmol) and iodomethane (357 mg, 2.52 mmol) in DMF (4 mL) was added potassium carbonate (348 mg, 2.52 mmol). The reaction was heated at 50° C. for 2 h, then diluted with EtOAc (30 mL), and washed with brine (3×10 mL). The organic layers were combined and concentrated to dryness and the resulting residue was purified by preparative TLC (100% EtOAc) to afford 5-chloro-1-methyl-3-nitro-1H-pyrazolo[4,3-b]pyridine (200 mg, 37%) as a yellow solid. MS (ESI): Mass calcd. for C₇H₅ClN₄O₂, 212.0; m/z found, 213.0 [M+H]⁺.

### Intermediate 13: 8-Chloro-2-(methylthio)pyrido[3,4-d]pyrimidine

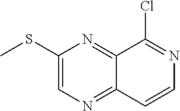

Step A: Methyl (E)-5-(2-ethoxyvinyl)-2-(methylthio)pyrimidine-4-carboxylate. A mixture of methyl-5-bromo-2-(methylthio)pyrimidine-4-carboxylate (3.00 g, 11.4 mmol), (E)-2-(2-ethoxyvinyl)-4,4,5,5-tetramethyl-1,3,2-dioxaborolane (2.26 g, 11.40 mmol), Pd(dppf)Cl₂ (0.43 g, 0.57 mmol), water (10 mL) and Na₂CO₃ (3.63 g, 34.2 mmol) in THF (70 mL) was heated at 65° C. for 16 h under an Ar atmosphere. The reaction mixture was concentrated to dryness and the residue was purified by FCC eluting with petroleum ether/ethyl acetate (20/1 to 8/1, gradient elution) to afford methyl (E)-5-(2-ethoxyvinyl)-2-(methylthio)pyrimidine-4-carboxylate (1.2 g, 41%) as a yellow solid. MS (ESI): Mass calcd. for C₁₁H₁₄N₂O₃S, 254.1; m/z found, 255.1 [M+H]⁺.

Step B: (E)-5-(2-Ethoxyvinyl)-2-(methylthio)pyrimidine-4-carboxamide. A mixture of methyl (E)-5-(2-ethoxyvinyl)-2-(methylthio)pyrimidine-4-carboxylate (1.2 g, 4.7 mmol) in a solution of NH₃ in MeOH (7 M, 12 mL, 84 mmol) was heated at 80° C. for 16 h in a sealed tube. The reaction mixture was cooled to rt and then concentrated under reduced pressure to afford (E)-5-(2-ethoxyvinyl)-2-(methylthio)pyrimidine-4-carboxamide (950 mg, 84%) as a yellow solid, which was used in the next step without further purification. MS (ESI): Mass calcd. for C₁₀H₁₃N₃O₂S, 239.1; m/z found, 240.1 [M+H]⁺.

Step C: 2-(Methylthio)pyrido[3,4-d]pyrimidin-8-ol. To a solution of (E)-5-(2-ethoxyvinyl)-2-(methylthio)pyrimidine-4-carboxamide (950 mg, 3.97 mmol) in toluene (25 mL), was added p-toluenesulfonic acid monohydrate (113 mg, 0.60 mmol). The resulting mixture was heated at 90° C. for 2 h, cooled to rt and then concentrated under reduced pressure to afford 2-(methylthio)pyrido[3,4-d]pyrimidin-8-ol (700 mg, 91%) as a yellow solid, which was used in the next step without further purification. MS (ESI): Mass calcd. for C₈H₇N₃OS, 193.0; m/z found, 194.1 [M+H]⁺.

Step D: 8-Chloro-2-(methylthio)pyrido[3,4-d]pyrimidine. A mixture of 2-(methylthio)pyrido[3,4-d]pyrimidin-8-ol (700 mg, 3.62 mmol) in POCl₃ (25 mL, 269 mmol) was heated at 70° C. for 16 h. The mixture was concentrated under reduced pressure and the residue was partitioned between EtOAc (20 mL) and saturated aqueous NaHCO₃ solution (20 mL). The aqueous layer was extracted with EtOAc (2×10 mL) and the combined organic solvent extracts were washed with brine (20 mL), dried over Na₂SO₄, filtered and concentrated. The residue was purified by preparative TLC (petroleum ether/ethyl acetate=3/1, R_{f}=0.4) to afford 8-chloro-2-(methylthio)pyrido[3,4-d]pyrimidine (500 mg, 65%) as white solid. MS (ESI): Mass calcd. for C₈H₆ClN₃S, 211.0; m/z found, 212.1 [M+H]⁺.

### Intermediate 14: Methyl 6-(3-iodophenyl)picolinate

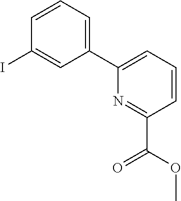 [SMILES: COC(=O)c1cccc(-c2cccc(I)c2)n1]

A mixture of methyl 6-bromopicolinate (1.0 g, 4.63 mmol), (3-iodophenyl) boronic acid (1.0 g, 4.21 mmol), KF (733 mg, 12.6 mmol), PdCl₂(dppf) (128 mg, 0.17 mmol), 1,4-dioxane (15 mL) and water (2 mL) was heated at 80° C. under a N₂ atmosphere for 15 h. The mixture was concentrated, diluted with water (35 mL) and extracted with EtOAc (4×35 mL). The combined organic solvent extracts were washed with brine (20 mL), dried over Na₂SO₄, filtered and concentrated. The residue was purified by preparative TLC (petroleum ether/ethyl acetate=5/1) to afford methyl 6-(3-iodophenyl)picolinate (89 mg, 6.2%) as a yellow oil. MS (ESI): Mass calcd. for C₁₃H₁₀INO₂, 339.1; m/z found, 340.0 [M+H]⁺.

### Intermediate 15: 6-Chloro-4-methoxypicolinamide

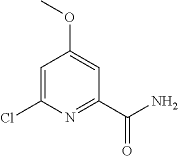

To a solution of 6-chloro-4-methoxypicolinic acid (200 mg, 1.07 mmol) in DCM (3 mL) and DMF (3 mL) was added ammonium chloride (160 mg, 2.99 mmol), followed by DIEA (689 mg, 5.33 mmol) and HATU (567 mg, 1.49 mmol). The reaction mixture was stirred at rt overnight and then diluted with water (20 mL). The aqueous phase was extracted with EtOAc (3×15 mL), concentrated to dryness and purified by FCC (petroleum ether/ethyl acetate=1:1) to afford 6-chloro-4-methoxypicolinamide (130 mg, 65%) as a yellow solid. MS (ESI): Mass calcd. for C₇H₇ClN₂O₂, 186.0; m/z found, 187.0 [M+H]⁺.

### Intermediate 16: 6-Chloro-2-methylpyrido[3,2-d]pyrimidin-4-amine

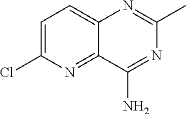

A vial containing 3-amino-6-chloropicolinonitrile (100 mg, 0.65 mmol) was charged with ethanimidamide hydrochloride salt (57.0 mg, 0.98 mmol), potassium phosphate tribasic (553 mg, 2.6 mmol), and THF (3 mL). The vial was sealed and heated at 80° C. for 16 h. The resulting mixture was cooled to rt and concentrated to dryness. To this residue was added water (3 mL) and the resulting mixture was heated at 70° C. After stirring for 30 min, the resulting mixture was cooled to rt and stirred for an additional 30 min. The resulting solid was isolated by filtration and washed sequentially with water (3 mL) and Et₂O (10 mL) to afford 6-chloro-2-methylpyrido[3,2-d]pyrimidin-4-amine (70 mg, 55%) as a pale yellow solid. MS (ESI): Mass calcd. for C₈H₇ClN₄, 194.0; m/z found, 195.0 [M+H]⁺. ¹H NMR (400 MHz, CDCl₃) δ 8.00 (d, J=8.7 Hz, 1H), 7.62 (d, J=8.7 Hz, 1H), 6.76 (s, 2H), 2.63 (s, 3H).

### Intermediate 17: 6-Chloropyrido[3,2-d]pyrimidin-2-d-4-amine

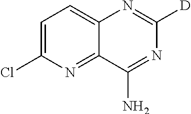 [SMILES: [2H]c1nc(N)c2nc(Cl)ccc2n1]

A 1 L round-bottomed flask was charged with 3-amino-6-chloropicolinonitrile (22.0 g, 0.14 mol), formamide-d₃ (20.6 g, 0.43 mol), K₃PO₄ (122 g, 0.57 mol), and cyclopentylmethyl ether (440 mL). The resultant mixture was heated at 65° C. for 16 h before cooling to rt. Then the reaction mixture was filtered and the filter cake was slurried in water (100 mL) at 20° C. for 3 h. The solid was isolated by filtration and dried to give 6-chloropyrido[3,2-d]pyrimidin-2-d-4-amine (23.9 g, 94%) as a yellow solid. MS (ESI): Mass calcd. for C₇H₄DClN₄, 181.0; m/z found, 182.0 [M+H]⁺. ¹H NMR (400 MHz, DMSO-d₆) δ 8.15 (d, J=8.8 Hz, 1H), 8.05 (br s, 1H), 7.95 (br s, 1H), 7.88 (d, J=8.8 Hz, 1H).

### Intermediate 18: 6-(3-Iodophenyl)pyrido[3,2-d]pyrimidin-2-d-4-amine

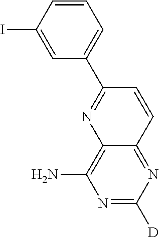 [SMILES: [2H]c1nc(N)c2nc(-c3cccc(I)c3)ccc2n1]

Step A: 6-(3-(Trimethylsilyl)phenyl)pyrido[3,2-d]pyrimidin-2-d-4-amine. A 1 L flask equipped with an overhead stirrer containing 6-chloropyrido[3,2-d]pyrimidin-2-d-4-amine (Intermediate 17, 31 g, 0.14 mol) was charged with 4-(trimethylsilyl) phenylboronic acid (32 g, 0.17 mol), Pd(dppf)Cl₂ (10.2 g, 13.8 mmol), CH₃CN (500 mL) and aqueous Cs₂CO₃ solution (1 M, 125 mL) under at atmosphere of N₂, successively. The resulting mixture was heated at 75° C. for 3 h. Water (375 mL) was added and the reaction mixture was further heated at 65° C. for 0.5 h. The resultant mixture was then allowed to cool to rt gradually and stirred for 1 h. The product was isolated by filtration and dried to afford 6-(3-(trimethylsilyl)phenyl)pyrido[3,2-d]pyrimidin-2-d-4-amine (39 g, 94%) as a brown solid. MS (ESI): Mass calcd. for C₁₆H₁₇DN₄Si, 295.1; m/z found, 296.1 [M+H]⁺. ¹H-NMR (300 MHz, DMSO-d₆) δ 8.46 (t, J=8.9 Hz, 2H), 8.39 (s, 1H), 8.15 (d, J=8.8 Hz, 1H), 8.04 (d, J=10.3 Hz, 2H), 7.65 (d, J=7.1 Hz, 1H), 7.54 (t, J=7.6 Hz, 1H), 0.35 (s, 9H).

Step B: 6-(3-Iodophenyl)pyrido[3,2-d]pyrimidin-2-d-4-amine. A 10-L round-bottom flask equipped with overhead stirrer was charged with 6-(3-(trimethylsilyl) phenyl) pyrido[3,2-d]pyrimidin-2-d-4-amine (49.0 g, 0.17 mol) and DCM (980 mL). A solution of ICI (134 g, 0.830 mol) in CH₂Cl₂ (735 mL) was then added dropwise at −5° C., the reaction mixture was warmed to 15° C. and stirred for 1 h. A precipitate formed and was isolated by filtration and dried under vacuum at 50° C. The resultant solid was combined with several additional batches of this material and was dissolved in DMSO (800 mL). Then an aqueous solution of K₂HPO₄ (10 wt %) was added dropwise to the above solution and stirring was continued at 20° C. for 2 h. The precipitate was isolated by filtration followed by slurrying in water (1600 mL) at 20° C. for 4 h. The solid was isolated and dried to afford 6-(3-iodophenyl)pyrido[3,2-d]pyrimidin-2-d-4-amine as light-brown solid (135 g). MS (ESI): Mass calcd. for C₁₃H₈DIN₄, 349.0; m/z found, 350.0 [M+H]⁺. ¹H-NMR (400 MHz, DMSO-d₆) δ 8.84 (m, 1H), 8.47 (m, 1H), 8.40 (m, 2H), 8.12 (m, 2H), 7.86 (m, 1H), 7.34 (m, 1H).

### Intermediate 19: 3-Amino-6-(3-iodophenyl)picolinamide

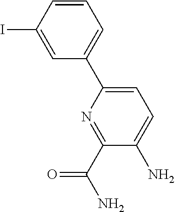 [SMILES: NC(=O)c1nc(-c2cccc(I)c2)ccc1N]

Step A: 3-Amino-6-(3-(trimethylsilyl)phenyl)picolinamide. To an N₂ purged solution of 3-amino-6-chloropicolinamide (1.00 g, 5.83 mmol) and (3-(trimethylsilyl)phenyl)boronic acid (1.11 g, 5.83 mmol) in dioxane (58 mL) and water (15 mL), was added (2-dicyclohexylphosphino-2′,6′-diisopropoxy-1,1′-biphenyl)[2-(2′-amino-1′-biphenyl)]palladium(II) methanesulfonate (0.48 g, 0.58 mmol) followed by Cs₂CO₃ (5.70 g, 17.5 mmol). The reaction mixture was heated at 80° C. for 1 h, partitioned between EtOAc (40 mL) and water (60 mL), and the organic layers were separated, concentrated to dryness and purified by FCC (0-40% EtOAc:hexanes) to afford 3-amino-6-(3-(trimethylsilyl)phenyl)picolinamide (1.2 g, 73%) as an orange solid. MS (ESI): Mass calcd. for C₁₅H₁₉N₃OSi, 285.1; m/z found, 286.1 [M+H]⁺. ¹H-NMR (400 MHz, DMSO-d₆) δ 8.12-8.03 (m, 2H), 8.02 (s, 1H), 7.87 (d, J=8.7 Hz, 1H), 7.49-7.44 (m, 1H), 7.43-7.37 (m, 2H), 7.25 (d, J=8.7 Hz, 1H), 6.95 (s, 2H), 0.30 (s, 9H).

Step B: 3-Amino-6-(3-iodophenyl)picolinamide. To a solution of 3-amino-6-(3-(trimethylsilyl)phenyl)picolinamide (0.20 g, 0.70 mmol) in DCM (7 mL) at 0° C. was added a solution of ICI (3.5 mL, 2 M in DCM, 3.50 mmol) dropwise. The reaction mixture was allowed to warm to rt and stirred for 1 h. A solution of saturated aqueous Na₂S₂O₃ (25 mL) was added and the solid was isolated by filtration. The solid was purified by FCC (0-10% MeOH:DCM) to afford 3-amino-6-(3-iodophenyl)picolinamide (133 mg, 56%) as a beige solid. MS (ESI): Mass calcd. for Cl₂H₁₀IN₃O, 339.0; m/z found, 340.0 [M+H]⁺. ¹H-NMR (400 MHz, DMSO-d₆) δ 8.45-8.39 (m, 1H), 8.16 (s, 1H), 8.12-8.01 (m, 1H), 7.87 (d, J=8.7 Hz, 1H), 7.70-7.62 (m, 1H), 7.40 (s, 1H), 7.25-7.17 (m, 2H), 7.03 (s, 2H).

### Intermediate 20: 2-(5-Iodo-2-methylphenyl)thiazolo[5,4-d]pyrimidin-7-amine

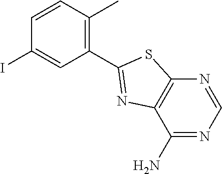 [SMILES: Cc1ccc(I)cc1-c1nc2c(N)ncnc2s1]

Step A: N-(4-Amino-6-oxo-1,6-dihydropyrimidin-5-yl)-5-iodo-2-methylbenzamide.

A 2 L round-bottomed flask equipped with an overhead stirrer was charged with 5,6-diaminopyrimidin-4(3H)-one (47.3 g, 375 mmol), 5-iodo-2-methylbenzoic acid (108 g, 412 mmol), DMF (710 mL), and DIEA (153 g, 1.18 mol), successively. The flask was purged with nitrogen and cooled to 0-10° C. before adding 1-[bis(dimethylamino)methylene]-1H-1,2,3-triazolo[4,5-b]pyridinium 3-oxide hexafluorophosphate (148 g, 390 mmol). The resultant mixture was stirred for 1 h at 0-10° C. before warming to rt with stirring for 18 h. The mixture was diluted with acetonitrile (709 mL) with continued stirring for 30 min. The resulting solid was filtered and washed with CH₃CN (190 mL×3). The filter cake was collected and dried under vacuum at 50-55° C. to afford N-(4-amino-6-oxo-1,6-dihydropyrimidin-5-yl)-5-iodo-2-methylbenzamide (108 g, 78.0%) as a light brown solid. ¹H NMR (400 MHz, DMSO-d₆) δ 11.68 (s, 1H), 8.89 (s, 1H), 8.01 (s, 1H), 7.78 (s, 1H), 7.66 (d, J=8.0 Hz, 1H), 7.05 (d, J=8.0 Hz, 1H), 6.39 (s, 2H), 2.35 (s, 3H).

Step B: 2-(5-Iodo-2-methylphenyl)thiazolo[5,4-d]pyrimidin-7-amine. A 2 L round-bottomed flask equipped with an overhead stirrer was charged with N-(4-amino-6-oxo-1,6-dihydropyrimidin-5-yl)-5-iodo-2-methylbenzamide (110 g, 297 mmol), pyridine (1.10 L), and P₂S₅ (165 g, 742 mmol). The resultant mixture was heated at 100° C. for 1 h before cooling to rt. The mixture was concentrated to dryness, diluted with acetonitrile (550 mL), and neutralized with 1 N aqueous HCl (1.20 L). The resulting mixture was stirred for 1 h, the suspension was filtered, washed with MeOH (110 mL×3) and dried under vacuum at 50-55° C. The resulting solid was further purified by adding MeOH (1150 mL) at 60° C. and stirring for 1 h. The solid was collected by filtration and dried under vacuum at 50-55° C. to afford 2-(5-iodo-2-methylphenyl)thiazolo[5,4-d]pyrimidin-7-amine (88.4 g, 80.8%) as a light yellow solid. ¹H NMR (300 MHz, DMSO-d₆) δ 8.33 (s, 1H), 8.15 (d, J=1.9 Hz, 1H), 7.81 (d, J=2.0 Hz, 2H), 7.78 (d, J=1.9 Hz, 1H), 7.23 (d, J=8.1 Hz, 1H), 2.57 (s, 3H).

### Intermediate 21: 2-(3-Iodophenyl)thiazolo[5,4-d]pyrimidin-7-amine

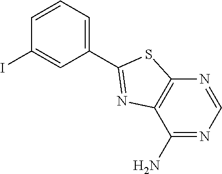 [SMILES: Nc1ncnc2sc(-c3cccc(I)c3)nc12]

Step A: N-(4-Amino-6-oxo-1,6-dihydropyrimidin-5-yl)-3-iodobenzamide. To a solution of 5,6-diaminopyrimidin-4-ol and NaOH (2 M, 50 mL, 100 mmol) at 0° C., was added 3-iodobenzoyl chloride (4.23 g, 15.9 mmol). The reaction mixture was stirred for 4 h at 0° C., allowed to warm to rt and then stirred overnight. The solid was removed by filtration and the pH of the filtrate was adjusted to pH 4-5 with acetic acid. The resulting precipitate was filtered, washed with water (50 mL), and the resulting solid was suspended in EtOH (100 mL) and stirred for 10 min and filtered. The filter cake was washed with EtOH (30 mL) and then dried to afford N-(4-amino-6-oxo-1,6-dihydropyrimidin-5-yl)-3-iodobenzamide (0.64 g, 23%) as a brown solid. MS (ESI): Mass calcd. for C₁₁H₉IN₄O₂, 356.0; m/z found, 357 [M+H]⁺.

Step B: 2-(3-Iodophenyl)thiazolo[5,4-d]pyrimidin-7-amine. A solution of N-(4-amino-6-oxo-1,6-dihydropyrimidin-5-yl)-3-iodobenzamide (640 mg, 1.80 mmol), 2,4-bis(4-methoxyphenyl)-2,4-dithioxo-1,3,2,4-dithiadiphosphetane, (727 mg, 1.80 mmol in toluene (15 mL) was heated to reflux for 16 h, cooled to rt and the solid was isolated by filtration. The filter cake was washed with toluene (5 mL), the solid was suspended in MeOH (3 mL), stirred for 2 min and isolated by filtration to afford 2-(3-iodophenyl)thiazolo[5,4-d]pyrimidin-7-amine (420 mg, 66%) as a white solid. MS (ESI): Mass calcd for C₁₁H₇IN₄S, 353.9; m/z found, 355.0 [M+H]⁺.

### Intermediate 22: 4-(Azetidin-1-yl)-6-chloropyrido[3,2-d]pyrimidine

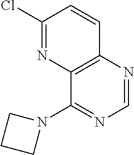 [SMILES: Clc1ccc2ncnc(N3CCC3)c2n1]

To a mixture of 4,6-dichloropyrido[3,2-d]pyrimidine (250 mg, 1.25 mmol), DIPEA (0.87 mL, 5.00 mmol), and DMF (2.5 mL), was added azetidine (71.3 mg, 1.25 mmol) and the resulting mixture was stirred at rt. After 1.5 h, the mixture was filtered and the filter cake was dried under reduced pressure to afford 4-(azetidin-1-yl)-6-chloropyrido[3,2-d]pyrimidine (200 mg, 73%). As a white solid. MS (ESI): Mass calcd. for C₁₀H₉ClN₄, 220.1; m/z found, 221.1 [M+H]⁺. ¹H NMR (400 MHz, CD₃OD) δ 8.34 (s, 1H), 7.97 (d, J=8.8 Hz, 1H), 7.69 (d, J=8.8 Hz, 1H), 4.88 (t, J=7.7 Hz, 2H), 4.36 (t, J=7.7 Hz, 2H), 2.82-2.21 (m, 2H).

### Intermediate 23: (R)-3-Hydroxy-3-(3-(3-iodophenyl)isoxazol-5-yl)-1-methylpyrrolidin-2-one

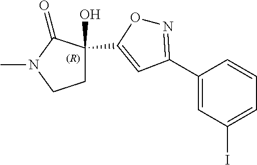

Step A: 3-Iodobenzaldehyde oxime. To a solution of 3-iodobenzaldehyde (1.5 g, 6.5 mmol) in EtOH (20 mL) was added NH₂OH (50% in water) (0.46 mL, 7.8 mmol). The mixture was stirred at 25° C. for 16 h, then concentrated to dryness to give 3-iodobenzaldehyde oxime (1500 mg, 6.072 mmol) as a pale yellow solid, which was used in the next step without further purification. MS (ESI): Mass calcd. for C₇H₆INO, 247.0; m/z found, 248.1 [M+H]⁺.

Step B: N-Hydroxy-3-iodobenzimidoyl chloride. N-chlorosuccinimide (973 mg, 7.29 mmol) was added portion-wise to a solution of 3-iodobenzaldehyde oxime (1.5 g, 6.1 mmol) in dry DCM (30 mL). The reaction mixture was stirred at 25° C. for 2 h. The mixture was then concentrated to give N-hydroxy-3-iodobenzimidoyl chloride (1450 mg, 5.151 mmol) as a pale yellow oil which was used in the next step without further purification. MS (ESI): Mass calcd. for C₇H₅ClINO, 280.9; m/z found, 282.3 [M+H]⁺.

Step C: (R)-3-Hydroxy-3-(3-(3-iodophenyl)isoxazol-5-yl)-1-methylpyrrolidin-2-one. To a solution of N-hydroxy-3-iodobenzimidoyl chloride (650 mg, 2.31 mmol) in DCM (15 mL) were added (R)-3-ethynyl-3-hydroxy-1-methylpyrrolidin-2-one (Intermediate 1, 385 mg, 2.77 mmol) and TEA (934 mg, 9.24 mmol). The mixture was heated at 50° C. for 4 h. The mixture was then cooled to rt, washed with brine (20 mL), dried over Na₂SO₄, filtered and concentrated. The residue was purified by preparative TLC (DCM/MeOH=20/1, R_{f}=0.4) to give (R)-3-hydroxy-3-(3-(3-iodophenyl)isoxazol-5-yl)-1-methylpyrrolidin-2-one (110 mg, 0.266 mmol) as a yellow solid. MS (ESI): Mass calcd. for C₁₄H₁₃IN₂O₃, 384.0; m/z found, 385.0 [M+H]⁺.

### Intermediate 24: tert-Butyl 3-(4,4,5,5-tetramethyl-1,3,2-dioxaborolan-2-yl)-1H-pyrrolo[2,3-b]pyridine-1-carboxylate

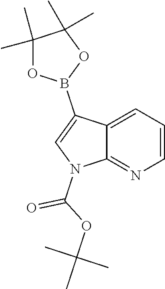 [SMILES: CC(C)(C)OC(=O)n1cc(B2OC(C)(C)C(C)(C)O2)c2cccnc21]

Step A: 3-Iodo-1H-pyrrolo[2,3-b]pyridine. To a solution of 1H-pyrrolo[2,3-b]pyridine (5 g, 42.324 mmol) in MeCN (60 mL) was added N-iodosuccinimide (11.427 g, 50.789 mmol), portion-wise. The mixture was stirred at 25° C. for 2 h, then the solid from the reaction mixture was collected by vacuum filtration. This solid was washed with MeCN (30 mL) and the filtrate was concentrated to give 3-iodo-1H-pyrrolo[2,3-b]pyridine (8.0 g, 31 mmol) as a yellow solid. MS (ESI): Mass calcd. for C₇H₅IN₂, 244.0; m/z found, 245.1 [M+H]⁺.

Step B: tert-Butyl-3-iodo-1H-pyrrolo[2,3-b]pyridine-1-carboxylate. To a solution of 3-iodo-1H-pyrrolo[2,3-b]pyridine (8.00 g, 32.8 mmol), DMAP (0.40 g, 3.28 mmol) and TEA (13.2 g, 131 mmol) in DCM (130 mL) was added di-tert-butyl dicarbonate (8.58 g, 39.3 mmol) dropwise. The mixture was stirred at 25° C. for 2 h. The reaction mixture was then diluted with DCM (130 mL) and washed with saturated aqueous Na₂SO₃ solution (100 mL) and then with brine (120 mL), dried over anhydrous Na₂SO₄, filtered and concentrated. The residue was purified by silica gel column chromatography eluting with petroleum ether/EtOAc (8/1) to give tert-butyl 3-iodo-1H-pyrrolo[2,3-b]pyridine-1-carboxylate (11.8 g) as a yellow oil. MS (ESI): Mass calcd. for Cl₂H₁₃₁N₂O₂, 344.0; m/z found, 345.1 [M+H]⁺.

Step C: tert-Butyl 3-(4,4,5,5-tetramethyl-1,3,2-dioxaborolan-2-yl)-1H-pyrrolo[2,3-b]pyridine-1-carboxylate. To a solution of tert-butyl 3-iodo-1H-pyrrolo[2,3-b]pyridine-1-carboxylate (11.8 g, 34.3 mmol) in 1,4-dioxane (50 mL) was added bis(pinacolato)diboron (17.4 g, 68.6 mmol), PdCl₂(dppf) (0.78 g, 1.03 mmol) and KOAc (10.1 g, 103 mmol). The mixture was heated at 85° C. for 16 h under an argon atmosphere. The mixture was cooled to room temperature, diluted with EtOAc (50 mL) and washed with brine (50 mL), dried over Na₂SO₄, filtered and concentrated to dryness. The residue was purified by silica gel column chromatography eluting with petroleum ether/EtOAc (gradient from 20/1 to 8/1) to give tert-butyl 3-(4,4,5,5-tetramethyl-1,3,2-dioxaborolan-2-yl)-1H-pyrrolo[2,3-b]pyridine-1-carboxylate (8 g, 9 mmol) as a pale yellow solid. MS (ESI): Mass calcd. for C₁₈H₂₅BN₂O₄, 344.2; m/z found, 345.3 [M+H]⁺.

### Intermediate 25: 6-(3-Chlorophenyl)pyrido[3,2-d]pyrimidin-2-d-4-amine

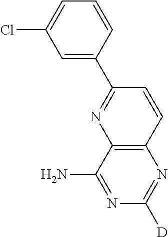 [SMILES: [2H]c1nc(N)c2nc(-c3cccc(Cl)c3)ccc2n1]

1,1′-Bis(di-tert butylphosphino)ferrocene palladium dichloride (144 mg, 0.22 mmol) was added to a mixture of 6-chloropyrido[3,2-d]pyrimidin-2-d-4-amine (Intermediate 17, 400 mg, 2.20 mmol), (3-chlorophenyl)boronic acid (413 mg, 2.64 mmol), K₃PO₄ (1.4 g, 6.6 mmol), 1,4-dioxane (6.4 mL), and H₂O (1.6 mL). The resultant mixture was sparged with Ar for 5 minutes and then subjected to microwave irradiation at 90° C. for 1.5 hours. The mixture was then cooled to room temperature and concentrated to dryness under reduced pressure to give the product, which was purified by FCC (eluent: petroleum ether:ethyl acetate=1:0 to 1:1) to afford 6-(3-chlorophenyl)pyrido[3,2-d]pyrimidin-2-d-4-amine (300 mg, 51%) as a yellow solid. MS (ESI): Mass calcd. for C₁₃H₈ClDN₄ 257.7 m/z found, 258.1 [M+1]⁺.

### Intermediate 26: (S)-3-(3-(3-Bromophenyl)isoxazol-5-yl)-3-hydroxy-1-methylpyrrolidin-2-one

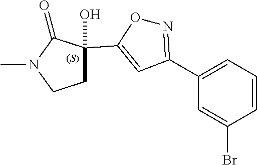

The title compound was prepared using analogous conditions described in Intermediate 3 using Intermediate 2 instead of Intermediate 1 in Step C. MS (ESI): Mass calcd. for C₁₄H₁₃BrN₂O₃, 336.0; m/z found, 337.1 [M+H]⁺. ¹H NMR (400 MHz, CD₃OD) δ 8.01 (t, J=1.8 Hz, 1H), 7.81 (td, J=7.8, 1.2 Hz, 1H), 7.67-7.61 (m, 1H), 7.41 (t, J=7.9 Hz, 1H), 6.92-6.89 (m, 1H), 3.62-3.49 (m, 2H), 2.95 (s, 3H), 2.72 (ddd, J=13.5, 6.8, 5.1 Hz, 1H), 2.45-2.33 (m, 1H).

### Intermediate 27: (S)-3-Hydroxy-1-methyl-3-(3-(3-(4,4,5,5-tetramethyl-1,3,2-dioxaborolan-2-yl)phenyl)isoxazol-5-yl)pyrrolidin-2-one

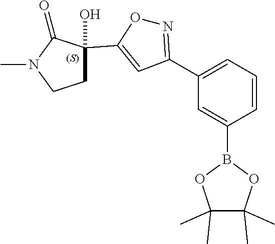

The title compound was prepared in a manner analogous to (R)-3-(3-(3-bromophenyl)isoxazol-5-yl)-3-hydroxy-1-methylpyrrolidin-2-one (Steps A-C) using (S)-3-ethynyl-3-hydroxy-1-methylpyrrolidin-2-one in Step C and the borylation reaction was analogous to (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₀H₂₅BN₂O₅, 384.2; m/z found, 385.2 [M+H]⁺.

### Intermediate 28: (R)-3-Hydroxy-1-methyl-3-(3-(3-methyl-5-(4,4,5,5-tetramethyl-1,3,2-dioxaborolan-2-yl)phenyl)isoxazol-5-yl)pyrrolidin-2-one

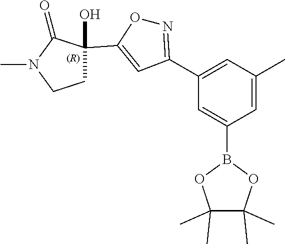

Step A: 3-Bromo-5-methylbenzaldehyde oxime. To a solution of 3-bromo-5-methylbenzaldehyde (2 g, 10.05 mmol) in EtOH (20 mL) was added NH₂OH (50% in water) (0.71 mL, 12.06 mmol). The mixture was stirred at 25° C. for 16 h and concentrated under reduced pressure to afford 3-bromo-5-methylbenzaldehyde oxime (2.1 g, 97%) as a white solid. MS (ESI): Mass calcd. for C₈H₈BrNO, 214.1; m/z found, 214.1 [M+H]⁺.

Step B: 3-Bromo-N-hydroxy-5-methylbenzimidoyl chloride. To a solution of 3-bromo-5-methylbenzaldehyde oxime (600 mg, 2.80 mmol) in anhydrous DCM (20 mL) was added N-chlorosuccinimide (450 mg, 3.36 mmol) portion wise. The reaction mixture was stirred at 25° C. for 2 h, filtered, and the filtrate was concentrated under reduced pressure to afford 3-bromo-N-hydroxy-5-methylbenzimidoyl chloride (0.6 g, 86%) as a yellow solid. MS (ESI): Mass calcd. for C₈H₇BrClNO, 248.5; m/z found, 249.1 [M+H]⁺.

Step C: (R)-3-(3-(3-Bromo-5-methylphenyl)isoxazol-5-yl)-3-hydroxy-1-methylpyrrolidin-2-one. A solution of (R)-3-ethynyl-3-hydroxy-1-methylpyrrolidin-2-one (Intermediate 1, 336 mg, 2.41 mmol), 3-bromo-N-hydroxy-5-methylbenzimidoyl chloride (600 mg, 2.41 mmol), and TEA (0.99 mL, 7.24 mmol) in DCM (20 mL) was stirred at 25° C. for 16 h. The reaction mixture was diluted with DCM (80 mL), washed with brine (60 mL), dried over anhydrous Na₂SO₄, filtered and concentrated. The residue was purified by prep-TLC (DCM/MeOH=20/1) to afford (R)-3-(3-(3-bromo-5-methylphenyl)isoxazol-5-yl)-3-hydroxy-1-methylpyrrolidin-2-one (0.3 g, 35%) as a yellow oil. MS (ESI): Mass calcd. for C₁₅H₁₅BrN₂O₃, 351.2; m/z found, 351.0 [M+H]⁺.

Step D: (R)-3-Hydroxy-1-methyl-3-(3-(3-methyl-5-(4,4,5,5-tetramethyl-1,3,2-dioxaborolan-2-yl)phenyl)isoxazol-5-yl)pyrrolidin-2-one. A mixture of bis(pinacolato)diboron (398 mg, 1.57 mmol), (R)-3-(3-(3-bromo-5-methylphenyl)isoxazol-5-yl)-3-hydroxy-1-methylpyrrolidin-2-one (275 mg, 0.78 mmol) Pd(dppf)Cl₂—CH₂Cl₂ (59.6 mg, 0.078 mmol), KOAc (230.5 mg, 2.35 mmol) and 1,4-dioxane (8 mL) was heated at 80° C. for 2 h. The reaction mixture was filtered and concentrated under reduced pressure. The residue was purified by FCC (DCM/MeOH=60/1) to afford (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one (0.3 g, 96%) as a yellow solid. MS (ESI): Mass calcd. for C₂₁H₂₇BN₂P₅, 398.3; m/z found, 399.3 [M+H]⁺.

### Intermediate 29: (R)-3-hydroxy-1-methyl-3-(3-(2-methyl-5-(4,4,5,5-tetramethyl-1,3,2-dioxaborolan-2-yl)phenyl)isoxazol-5-yl)pyrrolidin-2-one

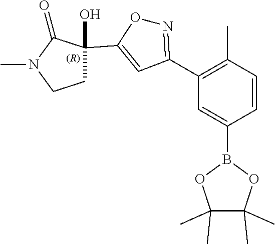

The title compound was prepared in a manner analogous to (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one Steps A-D using 5-bromo-2-methylbenzaldehyde in place of 3-bromo-5-methylbenzaldehyde in Step A. MS (ESI): Mass calcd. for C₂₁H₂₇BN₂O₅, 398.3; m/z found, 399 [M+H]⁺.

### Intermediate 30: (R)-3-Hydroxy-1-methyl-3-(3-(4-methyl-3-(4,4,5,5-tetramethyl-1,3,2-dioxaborolan-2-yl)phenyl)isoxazol-5-yl)pyrrolidin-2-one

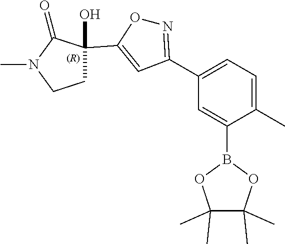

The title compound was prepared in a manner analogous to (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one Steps A-D using 3-bromo-4-methylbenzaldehyde in place of 3-bromo-5-methylbenzaldehyde in Step A. MS (ESI): Mass calcd. for C₂₁H₂₇BN₂O₅, 398.3; m/z found, 399 [M+H]⁺.

### Intermediate 31: (R)-3-Hydroxy-3-(3-(3-methoxy-5-(4,4,5,5-tetramethyl-1,3,2-dioxaborolan-2-yl)phenyl)isoxazol-5-yl)-1-methylpyrrolidin-2-one

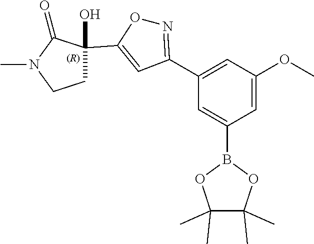

The title compound was prepared in a manner analogous to (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one Steps A-D using 3-bromo-5-methoxybenzaldehyde in place of 3-bromo-5-methylbenzaldehyde in Step A. MS (ESI): Mass calcd. for C₂₁H₂₇BN₂O₆, 414.2; m/z found, 415.2 [M+H]⁺.

### Intermediate 32: (R)-3-Hydroxy-1-methyl-3-(3-(5-(4,4,5,5-tetramethyl-1,3,2-dioxaborolan-2-yl)furan-2-yl)isoxazol-5-yl)pyrrolidin-2-one

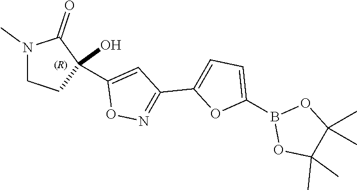

The title compound was prepared in a manner analogous to (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one Steps A-D using 5-bromofuran-2-carbaldehyde in place of 3-bromo-5-methylbenzaldehyde in Step A. MS (ESI): Mass calcd. for C₁₈H₂₃BN₂O₆, 374.2; m/z found, 293 [M −C₆H₁₂]⁺.

### Intermediate 33: (R)-3-(3-(5-(6-Chloropyridin-2-yl)furan-2-yl)isoxazol-5-yl)-3-hydroxy-1-methylpyrrolidin-2-one

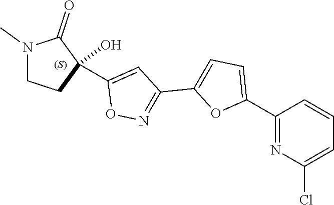

Step A: 5-(6-Chloropyridin-2-yl)furan-2-carbaldehyde. To a solution of 2-chloro-6-(furan-2-yl)pyridine (1.8 g, 10 mmol) in THF (40 mL) was added LDA (2 N, 6 mL, 12 mmol) and the reaction mixture was stirred at −78° C. for 1 h. A solution of DMF (1.5 g, 20 mmol) in THF (5 mL) was added dropwise and the reaction was stirred at −78° C. for 2 h and then diluted with water (50 mL). The mixture was allowed to warm to rt and extracted with EtOAc (2×50 mL). The combined organic layers were washed with brine (2×100 mL), dried over Na₂SO₄ and filtered. The filtrate was concentrated to dryness and then stirred with petroleum ether (50 mL) and EtOAc (5 mL) for 10 min.

The solid was isolated by filtration to afford 5-(6-chloropyridin-2-yl)furan-2-carbaldehyde (1.5 g, 72.0%) as a yellow solid. MS (ESI): Mass calcd. for C₁₀H₆CNO₂, 207.6; m/z found, 208 [M+H]⁺.

Step B: 5-(6-Chloropyridin-2-yl)furan-2-carbaldehyde oxime. To a solution of 5-(6-chloropyridin-2-yl)furan-2-carbaldehyde (1.3 g, 6.26 mmol) in EtOH (100 mL) was added hydroxylamine (50% in water, 827 mg, 12.52 mmol). The mixture was stirred at 25° C. for 6 h and then concentrated to afford 5-(6-chloropyridin-2-yl)furan-2-carbaldehyde oxime (1.3 g, 93%) as a yellow solid. MS (ESI): Mass calcd. for C₁₀H₇ClN₂O₂, 222.6; m/z found, 223.1 [M+H]⁺.

Step C: 5-(6-Chloropyridin-2-yl)-N-hydroxyfuran-2-carbimidoyl chloride. To a solution of 5-(6-chloropyridin-2-yl)furan-2-carbaldehyde oxime (500 mg, 2 mmol)) in dry DMF (8 mL) was added N-chlorosuccinimide (360 mg, 2.7 mmol) portion wise and the reaction was stirred at 30° C. for 4 hr. The reaction mixture was concentrated to dryness to afford 5-(6-chloropyridin-2-yl)-N-hydroxyfuran-2-carbimidoyl chloride (550 mg, 95%) as a yellow solid which was used in the next step without further purification. MS (ESI): Mass calcd. for C₁₀H₆Cl₂N₂O₂, 257.1; m/z found, 257 [M+H]⁺.

Step D: (R)-3-(3-(5-(6-Chloropyridin-2-yl)furan-2-yl)isoxazol-5-yl)-3-hydroxy-1-methylpyrrolidin-2-one. To a solution of 5-(6-chloropyridin-2-yl)-N-hydroxyfuran-2-carbimidoyl chloride (550 mg, 2.14 mmol) in DCM (30 mL) were added (R)-3-ethynyl-3-hydroxy-1-methylpyrrolidin-2-one (Intermediate 1, 298 mg, 2.14 mmol) and TEA (648 mg, 6.42 mmol). The mixture was stirred at 25° C. for 16 h. The mixture was washed with brine (2×50 mL), dried with Na₂SO₄ and filtered. The filtrate was concentrated and the residue was purified by Prep-TLC (UV-254, silica, DCM:MeOH/20:1) to afford (R)-3-(3-(5-(6-chloropyridin-2-yl)furan-2-yl)isoxazol-5-yl)-3-hydroxy-1-methylpyrrolidin-2-one (110 mg, 14%) as a brown oil. MS (ESI): Mass calcd. for C₁₇H₁₄ClN₃O₄, 359.8; m/z found, 360 [M+H]⁺.

### Intermediate 34: (R,S)-3-(3-(2-Bromopyridin-4-yl)isoxazol-5-yl)-3-hydroxy-1-methylpyrrolidin-2-one

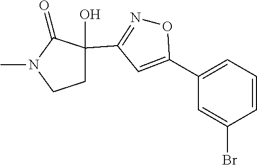 [SMILES: CN1CCC(O)(c2cc(-c3cccc(Br)c3)on2)C1=O]

The title compound (Step A: 700 mg, 30%; Step B: 1.4 g, 69%) was prepared using analogous conditions described in (R)-3-hydroxy-1-methyl-3-(5-(3-(4,4,5,5-tetramethyl-1,3,2-dioxaborolan-2-yl)phenyl)isoxazol-3-yl)pyrrolidin-2-one Steps A-B using (R,S)-3-ethynyl-3-hydroxy-1-methylpyrrolidin-2-one in place of (R)-3-ethynyl-3-hydroxy-1-methylpyrrolidin-2-one in Step A. MS (ESI): Mass calcd. for C₁₄H₁₃BrN₂O₃, 337.2; m/z found, 337 [M+H]⁺.

### Intermediate 35: (R,S)-3-(5-(3-Aminophenyl)isoxazol-3-yl)-3-hydroxy-1-methylpyrrolidin-2-one

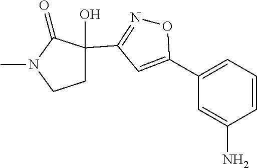 [SMILES: CN1CCC(O)(c2cc(-c3cccc(N)c3)on2)C1=O]

Step A: (R,S)-3-(5-(3-((Diphenylmethylene)amino)phenyl)isoxazol-3-yl)-3-hydroxy-1-methylpyrrolidin-2-one. A solution of (R,S)-3-(3-(2-bromopyridin-4-yl)isoxazol-5-yl)-3-hydroxy-1-methylpyrrolidin-2-one (Intermediate 34, 260 mg, 0.77 mmol), diphenylmethanimine (210 mg, 0.16 mmol), cesium carbonate (502 mg, 1.54 mmol), BINAP (72 mg, 0.12 mmol) and palladium(II) acetate (17 mg, 0.12 mmol) in dioxane (8 mL) was heated to 100° C. for 16 h. The reaction mixture was cooled to rt, concentrated to dryness and purified by FCC (DCM:MeOH, 20:1) to afford (R,S)-3-(5-(3-((diphenylmethylene)amino)phenyl)isoxazol-3-yl)-3-hydroxy-1-methylpyrrolidin-2-one (200 mg, 59% yield) as a yellow oil. MS (ESI): Mass calcd. for C₂₇H₂₃N₃O₃, 437.5; m/z found, 438 [M+H]⁺.

Step B: (R,S)-3-(5-(3-Aminophenyl)isoxazol-3-yl)-3-hydroxy-1-methylpyrrolidin-2-one. A solution of (R,S)-3-(5-(3-((diphenylmethylene)amino)phenyl)isoxazol-3-yl)-3-hydroxy-1-methylpyrrolidin-2-one (210 mg, 0.48 mmol) in EtOH (10 mL) was treated with aq. HCl (2.7 mL, 1 M, 2.7 mmol) and the reaction was stirred at rt for 1 h. The reaction mixture was diluted with sat. aq. NaHCO₃ (30 mL) and extracted with EtOAc (3×30 mL). The combined organic layers were dried (Na₂SO₄), filtered, concentrated and purified by prep TLC (DCM:MeOH, 15:1) to afford (R,S)-3-(5-(3-aminophenyl)isoxazol-3-yl)-3-hydroxy-1-methylpyrrolidin-2-one (95 mg, 72% yield) as a colorless solid. MS (ESI): Mass calcd. for C₁₄H₁₅N₃O₃, 273.1; m/z found, 274.2 [M+H]⁺.

### Intermediate 36: (R,S)-(3-(3-(3-Hydroxy-1-methyl-2-oxopyrrolidin-3-yl)isoxazol-5-yl)phenyl)boronic acid

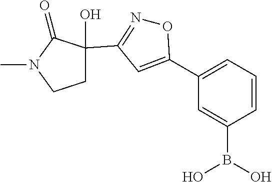 [SMILES: CN1CCC(O)(c2cc(-c3cccc(B(O)O)c3)on2)C1=O]

Step A: (R,S)-3-Hydroxy-1-methyl-3-(5-(3-(4,4,5,5-tetramethyl-1,3,2-dioxaborolan-2-yl)phenyl)isoxazol-3-yl)pyrrolidin-2-one. The title compound was prepared in a manner analogous to Step C of (R)-3-hydroxy-1-methyl-3-(5-(3-(4,4,5,5-tetramethyl-1,3,2-dioxaborolan-2-yl)phenyl)isoxazol-3-yl)pyrrolidin-2-one using (R,S)-3-(3-(2-bromopyridin-4-yl)isoxazol-5-yl)-3-hydroxy-1-methylpyrrolidin-2-one (Intermediate 34) in place of (R)-3-(5-(3-bromophenyl)isoxazol-3-yl)-3-hydroxy-1-methylpyrrolidin-2-one to afford (R,S)-3-hydroxy-1-methyl-3-(5-(3-(4,4,5,5-tetramethyl-1,3,2-dioxaborolan-2-yl)phenyl)isoxazol-3-yl)pyrrolidin-2-one (200 mg, 88% yield) as a yellow solid. MS (ESI): Mass calcd. for C₂₀H₂₅BN₂O₅, 384.2; m/z found, 385.2 [M+H]⁺.

Step B: (R,S)-(3-(3-(3-Hydroxy-1-methyl-2-oxopyrrolidin-3-yl)isoxazol-5yl)phenyl)boronic acid. The title compound was synthesized analogous to (R)-3-(5-(3-hydroxy-1-methyl-2-oxopyrrolidin-3-yl)isoxazol-3-yl)phenylboronic acid using (R,S)-3-hydroxy-1-methyl-3-(5-(3-(4,4,5,5-tetramethyl-1,3,2-dioxaborolan-2-yl)phenyl)isoxazol-3-yl)pyrrolidin-2-one to afford (R,S)-(3-(3-(3-hydroxy-1-methyl-2-oxopyrrolidin-3-yl)isoxazol-5yl)phenyl)boronic acid (40 mg, 61% yield) as a pale yellow solid. MS (ESI): Mass calcd. for C₁₄H₁₅BN₂O₅, 302.1; m/z found, 303.1 [M+H]⁺.

### Intermediate 37: (5R,8S)-2-(3-Formylphenyl)-6,7,8,9-tetrahydro-5H-5,8-epoxycyclohepta[d]pyrimidine-4-carboxamide

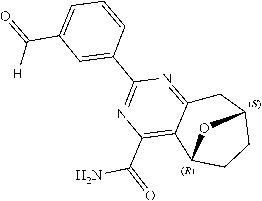

Step A: (5R,8S)-2-(3-Vinylphenyl)-6,7,8,9-tetrahydro-5H-5,8-epoxycyclohepta[d]pyrimidine-4-carboxamide. A mixture of (5R,8S)-2-(3-iodophenyl)-6,7,8,9-tetrahydro-5H-5,8-epoxycyclohepta[d]pyrimidine-4-carboxamide (350 mg, 0.86 mmol), potassium trifluoro(vinyl) borate (173 mg, 1.23 mmol), tetrakis(triphenylphosphine) palladium(0) (99 mg, 0.086 mmol), Na₂CO₃ (274 mg, 2.58 mmol), 1,4-dioxane (10 mL) and water (1 mL) was stirred under nitrogen at 85° C. for 16 h. The reaction mixture was diluted with water (15 mL) and extracted with EtOAc (3×20 mL). The organic layers were combined, dried over Na₂SO₄, filtered and concentrated. The product was purified by prep-TLC (DCM/MeOH=40/1, silica, UV=254 nm) to afford (5R,8S)-2-(3-vinylphenyl)-6,7,8,9-tetrahydro-5H-5,8-epoxycyclohepta[d]pyrimidine-4-carboxamide (250 mg, 82.5%) as a yellow solid. MS (ESI): Mass calcd. for C₁₈H₁₇N₃O₂, 307.3; m/z found, 308 [M+H]⁺.

Step B: (5R,8S)-2-(3-formylphenyl)-6,7,8,9-tetrahydro-5H-5,8-epoxycyclohepta[d]pyrimidine-4-carboxamide. To a solution of (5R,8S)-2-(3-vinylphenyl)-6,7,8,9-tetrahydro-5H-5,8-epoxycyclohepta[d]pyrimidine-4-carboxamide (250 mg, 0.81 mmol) in t-BuOH (4.5 mL), THF (3.0 mL) and H₂O (1.5 mL) was added N-methylmorpholine N-oxide (114.4 mg, 0.98 mmol) followed by OsO₄ (10 mg, 0.041 mmol). The reaction mixture was stirred at rt for 7 h and subsequently NaHCO₃ (820 mg, 9.76 mmol), NaIO₄ (557 mg, 2.60 mmol) and H₂O (3 mL) were added. The mixture was stirred for 45 min and then poured into aq. sat. Na₂SO₃. The mixture was extracted with diethyl ether. The organic layer was dried with MgSO₄, filtered and concentrated. The residue was purified by prep-TLC (DCM/MeOH=40/1, silica, UV=254 nm) to afford (5R,8S)-2-(3-formylphenyl)-6,7,8,9-tetrahydro-5H-5,8-epoxycyclohepta[d]pyrimidine-4-carboxamide (200 mg, 80%). MS (ESI): Mass calcd. for C₁₇H₁₅N₃O₃, 309; m/z found, 309.8 [M+H]⁺.

### Intermediate 38: 2-Chloro-7-fluoroquinazoline-4-carboxamide

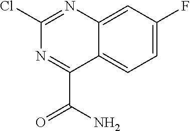 [SMILES: NC(=O)c1nc(Cl)nc2cc(F)ccc12]

Step A: 7-Fluoroquinazoline-2,4(1H,3H)-dione. An aqueous solution of potassium nitrate (10 mL, 4.8 M, 48.3 mmol) was added dropwise to a mixture of 2-amino-4-fluorobenzoic acid (5.0 g, 32.2 mmol) in AcOH (3.5 g, 58.0 mmol) and water (20 mL). The reaction mixture was heated to 50° C. for 16 h and then NaOH (5.1 g, 129 mmol) was added portionwise. The stirring was continued at 50° C. for 4 h, then the pH was slowly adjusted to pH<1 with conc. HCl_{(aq)}. The precipitate was isolated by filtration and washed with water to afford 7-fluoroquinazoline-2,4(1H,3H)-dione (2.8 g, 48% yield) as a yellow solid. MS (ESI): Mass calcd. for C₈H₅FN₂O₂, 180.0; m/z found, 212.1 [M+H]⁺.

Step B: 2,4-Dichloro-7-fluoroquinazoline. A mixture of 7-fluoroquinazoline-2,4(1H,3H)-dione (2.8 g, 15.5 mmol), POCl₃ (10 mL, 107.6 mmol), and DIEA (5.36 mL, 31.1 mmol) was heated to reflux for 4 h. The reaction mixture was concentrated in vacuo and treated with ice water. The precipitate was isolated by filtration, washed with water, and dried under reduced pressure to afford 2,4-dichloro-7-fluoroquinazoline (3.0 g, 89% yield) as a brown solid. MS (ESI): Mass calcd. for C₈H₃Cl₂FN₂, 217; m/z found, 217 [M+H].

Step C: 2-Chloro-4-(1-ethoxyvinyl)-7-fluoroquinazoline. To a solution of 2,4-dichloro-7-fluoroquinazoline (3.0 g, 13.8 mmol) in DMF (26 mL) was added tributyl(1-ethoxyvinyl)stannane (5.0 g, 13.8 mmol), followed by dichlorobis(triphenylphosphine)palladium(II) (0.48 g, 0.69 mmol). The reaction mixture was heated to 60° C. for 16 h, cooled to rt and quenched with KF_{(aq)}. EtOAc was added, the mixture was filtered, and the filtrate was washed with water, followed by brine. The product was purified by FCC (ether:EtOAc, 10:1) to afford 2-chloro-4-(1-ethoxyvinyl)-7-fluoroquinazoline (2.2 g, 63% yield) as a yellow solid. MS (ESI): Mass calcd. for Cl₂H₁₀ClFN₂O, 252.1; m/z found, 253 [M+H]⁺.

Step D: 2-Chloro-7-fluoroquinazoline-4-carboxylic acid. A solution of 2-chloro-4-(1-ethoxyvinyl)-7-fluoroquinazoline (2.2 g, 8.7 mmol) in dioxane (20 mL) was treated with a solution of sodium periodate (3.7 g, 17.4 mmol) in water (2 mL), followed by KMnO₄ (0.69 g, 4.4 mmol) and the mixture was stirred at rt 16 h. The pH was adjusted to 7-8 with sat. aq. K₂CO₃, and the precipitate was removed by filtration and washed with DCM. The filtrate was washed with water and the organic layer was isolated, dried, and concentrated under reduced pressure. The product was purified by prep TLC (ether:EtOAc, 3:1) to afford ethyl 2-chloro-7-fluoroquinazoline-4-carboxylate (900 mg, 41%). The pH of the aqueous layer was adjusted to pH 1-3 with aq 2 M HCl and extracted with DCM (2×20 mL). The organic layer was concentrated to afford 2-chloro-7-fluoroquinazoline-4-carboxylic acid (0.26 g, 13% yield) as a yellow solid. MS (ESI): Mass calcd. for C₉H₄ClFN₂O₂, 226; m/z found, 226 [M+H]⁺.

Step E: 2-Chloro-7-fluoroquinazoline-4-carboxamide. A solution of 2-chloro-7-fluoroquinazoline-4-carboxylic acid (150 mg, 0.66 mmol) in DCM (2 mL) was treated with oxalyl chloride (0.17 mL, 1.98 mmol) and DMF (0.036 mL, 0.47 mmol) at 0° C. The solution was stirred for 0.5 h and then treated with 28% NH₃ in water (0.15 mL, 2.29 mmol) and continued stirring at 0° C. for 0.5 h. The mixture was diluted with water (10 mL) and extracted with DCM (3×5 mL). The combined organic extracts were concentrated to afford 2-chloro-7-fluoroquinazoline-4-carboxamide (60 mg, 40% yield) as a yellow solid. MS (ESI): Mass calcd. for C₉HClFN₃O, 225.0; m/z found, 226 [M+H]⁺.

### Intermediate 39: Ethyl 2-chloro-7-fluoroquinazoline-4-carboxylate

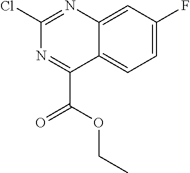 [SMILES: CCOC(=O)c1nc(Cl)nc2cc(F)ccc12]

The title compound was prepared in a manner analogous to Steps A-D of 2-chloro-7-fluoroquinazoline-4-carboxamide to afford ethyl 2-chloro-7-fluoroquinazoline-4-carboxylate (900 mg, 41%). MS (ESI): Mass calcd. for C₁₁H₈ClFN₂O₂, 254.7; m/z found, 255 [M+H]⁺.

### Intermediate 40: 6-(Tributylstannyl)picolinamide

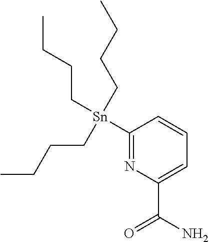 [SMILES: CCC[CH2][Sn]([CH2]CCC)([CH2]CCC)[c]1cccc(C(N)=O)n1]

To a solution of 6-bromopicolinamide (0.50 g, 2.49 mmol) in toluene (10 mL) was added tetrakis(triphenylphosphine)palladium(0) (0.14 g, 0.12 mmol) followed by bis(tributyltin) (2.89 g, 4.96 mmol) and the mixture was heated at 100° C. for 16 h. The reaction mixture was cooled to rt, concentrated to dryness and purified by prep-TLC (DCM:EtOAC 4:1) to afford 6-(tributylstannyl)picolinamide (0.80 g, 78% yield) as a colorless oil. MS (ESI): Mass calcd. for C₁₈H₃₂N₂OSn, 412.2; m/z found, 413 [M+H]⁺.

### Intermediate 41: 3-Amino-6-(tributylstannyl)picolinamide

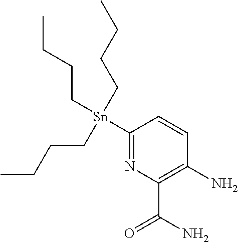 [SMILES: CCC[CH2][Sn]([CH2]CCC)([CH2]CCC)[c]1ccc(N)c(C(N)=O)n1]

The title compound was synthesized using analogous conditions to 6-(tributylstannyl)picolinamide using 3-amino-6-bromopicolinamide in place of 6-bromopicolinamide to afford 3-amino-6-(tributylstannyl)picolinamide (0.11 g, 46% yield) as a colorless oil. MS (ESI): Mass calcd. for C₁₈H₃₃N₃OSn, 427.2; m/z found, 427.0 [M+H]⁺.

### Intermediate 42: (R)-3-Hydroxy-1-methyl-3-(3-(2-(tributylstannyl)pyridin-4-yl)isoxazol-5-yl)pyrrolidin-2-one

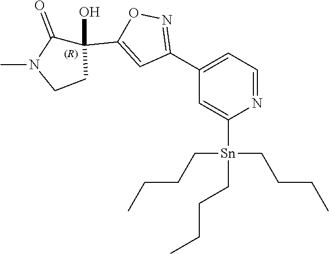

The title compound was synthesized using analogous conditions to 6-(tributylstannyl)picolinamide using (R)-3-(3-(2-bromopyridin-4-yl)isoxazol-5-yl)-3-hydroxy-1-methylpyrrolidin-2-one (Intermediate 7) in place of 6-bromopicolinamide to afford (R)-3-hydroxy-1-methyl-3-(3-(2-(tributylstannyl)pyridin-4-yl)isoxazol-5-yl)pyrrolidin-2-one (0.25 g, 22% yield) as a brown oil. MS (ESI): Mass calcd. for C₂₅H₃₉N₃O₃Sn, 549.2; m/z found, 550.0 [M+H]⁺.

### Intermediate 43: (R)-3-(3-(6-bromopyridin-2-yl)isoxazol-5-yl)-3-hydroxy-1-methylpyrrolidin-2-one

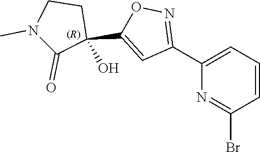

Step A. (E)-6-Bromopicolinaldehyde oxime. Into a 1 L 3-necked round-bottom flask was added 6-bromopyridine-2-carbaldehyde (40 g, 215 mmol), EtOH (600 mL) and NH₂OH·HCl (17.93 g, 258.05 mmol) at room temperature. To the above mixture was added sodium acetate (35.28 g, 430.09 mmol) in portions at 0° C. The resulting mixture was stirred overnight at room temperature and then concentrated under reduced pressure. The mixture was acidified to pH=7 with saturated aqueous NaHCO₃ (100 mL). The resulting mixture was extracted with EtOAc (2×100 mL). The combined organic extracts were washed with brine (1×150 mL) and dried over anhydrous Na₂SO₄. After filtration, the filtrate was concentrated under reduced pressure to a residue. The residue was purified by trituration with hexane (80 mL). The precipitated solids were collected by filtration and washed with hexanes to afford the title compound as a white solid (39 g, 90%).

Step B. (Z)-6-Bromo-N-hydroxypicolinimidoyl chloride. Into a 2 L 4-necked round-bottom flask was added (E)-N-[(6-bromopyridin-2-yl)methylidene]hydroxylamine (39 g, 194 mmol) and DMF (1 L) at room temperature. To the above mixture was added NCS (31.09 g, 232.8 mmol) at room temperature. The resulting mixture was stirred overnight at room temperature. Then the pH of the mixture was adjusted to pH=7 with TFA (5 L, 1 M). The resulting mixture was extracted with MTBE (2×1 L). The combined organic extracts were washed with brine (1×1 L) and dried over anhydrous Na₂SO₄. After filtration, the filtrate was concentrated under reduced pressure to provide a residue. The residue was purified by trituration with hexanes (80 mL). The precipitated solids were collected by filtration and washed with hexane to afford the title compound as a white solid (41.2607 g, 90.19%). LC-MS (ESI): Mass calcd. for C₆H₄BrClN₂O 233.9 m/z found 235 [M+H]⁺. ¹H NMR (300 MHz, DMSO-d₆) δ 12.89 (s, 1H), 7.91 (d, J=7.7, 1.2 Hz, 1H), 7.85 (t, J=7.6 Hz, 1H), 7.76 (d, J=7.6, 1.2 Hz, 1H).

Step C. (R)-3-(3-(6-Bromopyridin-2-yl)isoxazol-5-yl)-3-hydroxy-1-methylpyrrolidin-2-one. To a solution of (R)-3-ethynyl-3-hydroxy-1-methylpyrrolidin-2-one (90.0 g, 646.78 mmol, Intermediate 1) and (Z)-6-bromo-N-hydroxypicolinimidoyl chloride (609 g, 2.59 mol) in EtOAc (900 mL) and H₂O (450 mL) was added NaHCO₃ (326 g, 3.88 mol, in 150 mL H₂O). The mixture was stirred at 25° C. for 24 h and then filtered and concentrated under reduced pressure to give a residue. The residue was purified by column chromatography (petroleum ether/ethyl acetate=1/0 to 0/1). The product was triturated with EtOH (4000 mL) at 25° C. for 12 hr. The above procedure was repeated 4 times. This resulting product was triturated with petroleum ether:ethyl acetate=1:1 (2000 mL) at 25° C. for 12 h and repeated twice to afford the title compound as a white solid (490 g, 50.4%). LC-MS (ESI): Mass calcd. for C₁₃H₁₂BrN₃O₃, 337.01; m/z found, 340.0 [M+H]⁺. ¹H NMR (400 MHz, DMSO-d₆) δ 8.04 (d, J=7.5 Hz, 1H), 7.92 (t, J=7.8 Hz, 1H), 7.79 (d, J=7.9 Hz, 1H), 6.94 (s, 1H), 6.77 (s, 1H), 3.52-3.38 (m, 2H), 2.83 (s, 3H), 2.59 (ddd, J=4.9, 7.8, 13.2 Hz, 1H), 2.27 (ddd, J=5.8, 7.9, 13.5 Hz, 1H).

### Intermediate 44: (R)-3-Ethynyl-4,4-difluoro-3-hydroxy-1-methylpyrrolidin-2-one

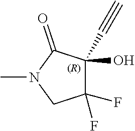

Step A. (3E)-1-Methyl-3-[(1-phenylethyl)imino]pyrrolidin-2-one. Into a 20 L 4-necked round-bottom flask was added 1-methylpyrrolidine-2,3-dione (346 g, 3060 mmol), MgSO₄ (368 g, 3060 mmol) and DCM (7 L). To this was added (+/−)-α-methylbenzylamine (389.21 g, 3211.74 mmol) and TFA (0.2 mL). The resulting mixture was stirred overnight at 40° C. under a nitrogen atmosphere. The mixture was allowed to cool to room temperature. The solids were filtered out and washed with DCM (1×1 L). The filtrate was collected and concentrated under reduced pressure. The product was slurried with ether (1.5 L). The solids were collected by filtration to afford (3E)-1-methyl-3-[(1-phenylethyl)imino]pyrrolidin-2-one (540 g) as a purple solid. ¹H NMR (300 MHz, DMSO-d₆) δ 7.39-7.08 (m, 5H), 5.41 (d, J=7.0 Hz, 1H), 4.88 (t, J=2.4 Hz, 1H), 4.30-4.13 (m, 1H), 3.76-3.52 (m, 2H), 2.89 (s, 3H), 1.40 (d, J=6.8 Hz, 3H).

Step B. 4,4-Difluoro-3,3-dihydroxy-1-methylpyrrolidin-2-one. Into a 10 L 4-necked round-bottom flask were added (3E)-1-methyl-3-[(1-phenylethyl)imino]pyrrolidin-2-one (540 g, 2496.71 mmol), Na₂SO₄ (511 g, 3600 mmol) and acetonitrile (5.4 L). To this was added Select-F (1946 g, 5493 mmol) in portions at 0° C. The reaction mixture was stirred overnight at room temperature and then acetonitrile (5.4 L) was added. To the mixture was added HCl in 1,4-dioxane (4 N, 936 mL) dropwise at 0° C. The resulting mixture was stirred for an additional 1 h at room temperature. The resulting precipitate was filtered and washed with acetonitrile (1×2 L). The filtrate was concentrated under vacuum. This filtration and concentration were repeated 2 times again. The residue was purified by DAC using a C18 column (CH₃CN/H₂O (0.1% NH₄HCO₃)=1% to 15% in 17 min) to afford 4,4-difluoro-3,3-dihydroxy-1-methylpyrrolidin-2-one (122 g, 23.9% Steps A and B) as an off-white solid. ¹H NMR (300 MHz, DMSO-d₆) δ 7.29 (s, 2H), 3.65 (t, J=12.0 Hz, 2H), 2.80 (s, 3H).

Step C. (3R)-3-Ethynyl-4,4-difluoro-3-hydroxy-1-methylpyrrolidin-2-one. Into a 5 L 4-necked round-bottom flask were added 4,4-difluoro-3,3-dihydroxy-1-methylpyrrolidin-2-one (122 g, 730.04 mmol), DMF (2440 mL), K₂CO₃ (10.09 g, 73.00 mmol), trimethylsilylacetylene (143 g, 1460 mmol) and Cu(OAc)₂ (26.52 g, 146.01 mmol) at room temperature under N₂ atmosphere. The resulting mixture was stirred for overnight at 50° C. The mixture was allowed to cool down to room temperature, quenched by the addition of MeOH (500 mL) at room temperature and concentrated under reduced pressure. The residue was purified by silica gel column chromatography, eluted with PE/EA (10:1 to 1:1) to afford (R,S)-3-ethynyl-4,4-difluoro-3-hydroxy-1-methylpyrrolidin-2-one (97 g, 75%) as a yellow solid. The (R) and (S) enantiomers of 3-ethynyl-4,4-difluoro-3-hydroxy-1-methylpyrrolidin-2-one (97 g and 6.2 g from a separate synthesis) were separated by Chiral-Prep-SFC (CHIRALPAK® IC-3 (50×4.6 mm), 80% hexanes with 0.1% DEA:20% EtOH) which provided as the first eluting enantiomer, (3R)-3-ethynyl-4,4-difluoro-3-hydroxy-1-methylpyrrolidin-2-one (45.6 g, 88%), as an off-white solid. LC-MS (ESI): Mass calcd. for C₇H₇F₂NO₂ 175.0 m/z found 176 [M+H]⁺. ¹H NMR (300 MHz, DMSO-d₆) δ 7.43 (s, 1H), 3.84-3.80 (m, 3H), 2.82 (s, 3H).

### Intermediate 45: (3R,5S)-3-ethynyl-3-hydroxy-1-methyl-5-(trifluoromethyl)pyrrolidin-2-one

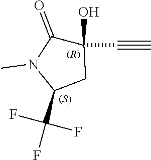

Step A. Ethyl 4,4,4-trifluoro-3-(methylamino)butanoate. To a solution of ethyl (E)-4,4,4-trifluorobut-2-enoate (4400 g, 26.17 mol, 3.89 L) in THF (18.0 L), was added MeNH₂ (3330 g, 32.17 mol, 30% purity). The light yellow solution was stirred at 25° C. for 3 hrs. The reaction mixture was concentrated under reduced pressure to provide the product as a yellow liquid (4.64 kg, 89%) which was used in the next step without further purification. ¹H NMR (400 MHz, chloroform-d) δ 4.08-4.26 (m, 2H), 3.40-3.56 (m, 1H), 2.61-2.68 (m, 1H), 2.51-2.55 (m, 3H), 2.41-2.50 (m, 1H), 1.22-1.29 (m, 3H).

Step B. Ethyl 4-hydroxy-1-methyl-5-oxo-2-(trifluoromethyl)-2,5-dihydro-1H-pyrrole-3-carboxylate. To a solution of ethyl 4,4,4-trifluoro-3-(methylamino)butanoate (2400 g, 12.05 mol) in 2-MeTHF (24000 mL) was added t-BuOK (1.35 kg, 12.05 mol) and diethyl oxalate (1.76 kg, 12.05 mol, 1.65 L) at 25° C. under N₂ atmosphere. The reaction mixture was heated at 60° C. for 3 h under a N₂ atmosphere. The reaction mixture was quenched by the addition of NH₄Cl (saturated aqueous, 10.0 L) at 25° C. Then, the pH of the mixture was adjusted to pH=2-3 with 1 M aqueous HCl (9.00 L), and the resulting mixture was extracted with EtOAc (5.00 L) twice. The combined organic extracts were washed with brine (15.0 L), dried over Na₂SO₄, filtered and concentrated under reduced pressure to give a residue. The residue was purified by column chromatography (DCM/MeOH 0% to 3%) to afford the title compound as a brown oil (4.20 kg, 16.59 mol, 68.8%). ¹H NMR (400 MHz, chloroform-d) δ 4.63 (q, J=5.0 Hz, 1H), 4.24-4.42 (m, 4H), 3.10-3.14 (m, 3H), 1.19-1.26 (m, 2H).

Step C. 1-Methyl-5-(trifluoromethyl)pyrrolidine-2,3-dione. A mixture of ethyl 4-hydroxy-1-methyl-5-oxo-2-(trifluoromethyl)-2,5-dihydro-1H-pyrrole-3-carboxylate (2000 g, 7.90 mol) in HCl (10.96 kg, 108.23 mol, 10.75 L, 36% solution) was heated at 110° C. for 16 hrs. The reaction mixture was cooled to 20° C. and extracted with isopropylacetate (5000 mL×8). The combined organic extracts were washed with brine (10000 mL), dried over Na₂SO₄, filtered and concentrated under reduced pressure to give a residue. The residue was triturated with MTBE (5000 mL) at 25° C. for 3 hrs. The solid was filtered, washed with MTBE (1000 mL) and dried under high vacuum to afford the title compound as a yellow solid (1.15 kg, 6.35 mol, 40.2%). ¹H NMR (400 MHz, chloroform-d) δ 4.28-4.42 (m, 1H), 3.28 (s, 3H), 2.87-3.01 (m, 1H), 2.71-2.83 (m, 1H).

Step D. 3-Hydroxy-1-methyl-5-(trifluoromethyl)-3-((trimethylsilyl)ethynyl)pyrrolidin-2-one. To a solution of trimethylsilylacetylene (1.21 kg, 12.3 mol, 1.71 L) in THF (5400 mL) was added dropwise, n-BuLi (2.5 M, 4.95 L) at −70 to −60° C. over 30 mins and the mixture was stirred at −70 to −60° C. for 1 hr. 1-Methyl-5-(trifluoromethyl)pyrrolidine-2,3-dione (1120 g, 6.18 mol) in THF (9000 mL) was added dropwise to the mixture at −70 to −60° C. over 30 mins. The black solution was stirred at −70 to −60° C. for 2 hrs. The reaction mixture was added into NH₄Cl (aqueous saturated, 10000 mL) drop wise at 0° C., then the pH of the mixture was adjusted to pH=5 with 2 M aqueous HCl (5000 mL) and the aqueous solution was extracted with EtOAc (5000 mL×3). The combined organic extracts were washed with 5000 mL brine, dried over Na₂SO₄, filtered and evaporated to dryness. The title compound was obtained as a red solid (1.50 kg, 86%) and was used without further purification. ¹H NMR (400 MHz, chloroform-d) δ 4.08-4.16 (m, 1H), 2.96-3.06 (m, 3H), 2.72-2.82 (m, 1H), 2.22-2.35 (m, 1H), 0.08-0.21 (m, 8H).

Step E. (3R,5S)-3-Ethynyl-3-hydroxy-1-methyl-5-(trifluoromethyl)pyrrolidin-2-one. 3-Hydroxy-1-methyl-5-(trifluoromethyl)-3-((trimethylsilyl)ethynyl)pyrrolidin-2-one (1500 g, 5.37 mol) was dissolved in MeOH (10.0 L), then K₂CO₃ (742 g, 5.37 mol) was added. The black mixture was stirred at 25° C. for 1 hr. The reaction mixture was filtered and the filter cake was washed with CH₂Cl₂ (4000 mL) and poured into water (3000 mL). The organic phase was separated, the aqueous phase was extracted with CH₂Cl₂ (3000 mL×2), the combined organic extracts were washed with water (2000 mL) and brine (2000 mL), dried over Na₂SO₄, and concentrated to dryness under reduced pressure to afford a mixture of (3R,5S)-3-ethynyl-3-hydroxy-1-methyl-5-(trifluoromethyl)pyrrolidin-2-one and (3S,5R)-3-ethynyl-3-hydroxy-1-methyl-5-(trifluoromethyl)pyrrolidin-2-one This mixture was purified by column chromatography (PE:EA=10:1 to 3:1) to afford a mixture of diastereomers (635 g, 55%, 97.8% purity). (3R,5S)-3-Ethynyl-3-hydroxy-1-methyl-5-(trifluoromethyl)pyrrolidin-2-one and (3S,5R)-3-ethynyl-3-hydroxy-1-methyl-5-(trifluoromethyl)pyrrolidin-2-one were then separated by SFC (column: DAICEL CHIRALPAK® AD (250 mm×50 mm, 10 m); mobile phase: [0.1% NH₃·H₂O EtOH]; B %: 20%-20%, min) to afford the title compound, (3R,5S)-3-ethynyl-3-hydroxy-1-methyl-5-(trifluoromethyl)pyrrolidin-2-one, the first eluting peak, as a yellow solid (95.0 g, 45.7%). ¹H NMR (400 MHz, chloroform-d) δ 3.97-4.08 (m, 1H), 3.03 (d, J=1.0 Hz, 3H), 2.80 (dd, J=13.6, 7.5 Hz, 1H), 2.62 (s, 1H), 2.33 (dd, J=13.7, 7.2 Hz, 1H), 1.23-1.28 (m, 1H).

### Intermediate 46: (R)-3-Ethynyl-3-hydroxy-1-methylpyrrolidin-2-one-4,4,5,5-d₄

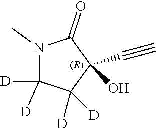

Step A: 1-Methylpyrrolidin-2-one-3,3,4,4,5,5-d₆. Into a 1000-mL, 3-necked round-bottom flask, purged and maintained with an atmosphere of nitrogen, was placed NaH (60% in mineral oil, 52 g, 1302 mmol). THF (400 mL) was added and the mixture was then cooled to 0° C. To this was added pyrrolidin-2-one-3,3,4,4,5,5-d₆ (39.5 g, 434 mmol) at 0° C. The resulting solution was stirred at 0° C. for 20 min, then iodomethane (184 g, 1302 mmol) was added dropwise at 0° C. The resulting solution was slowly warmed to room temperature and stirred for 3.5 hours. The resulting solution was poured into ice-water (800 mL), extracted with DCM (15×400 mL), the organic extracts combined, dried over anhydrous magnesium sulfate, filtered and concentrated under reduced pressure to afford 1-methylpyrrolidin-2-one-3,3,4,4,5,5-d₆ as a yellow oil (67.6 g) of LC-MS (ESI): Mass calcd. for C₅H₃D₆NO 105.1 m/z found 106 [M+H]⁺.

Step B: 3-Benzoyl-1-methylpyrrolidin-2-one-4,4,5,5-d₄. Into a 2000-mL, 3-necked round-bottom flask, purged and maintained with an atmosphere of nitrogen, was added NaH (60% in mineral oil, 26 g, 651 mmol), toluene (900 mL) and methanol (1.82 mL). To this was added a mixture solution of 1-methylpyrrolidin-2-one-3,3,4,4,5,5-d₆ (67.6 g, 434 mmol) and methyl benzoate (65 g, 477 mmol) dropwise at room temperature. The resulting solution was heated at 110° C. overnight. After this time, the reaction mixture was cooled to 0° C. and HOAc (39 g, 651 mmol) and water (24 g, 1302 mmol), were added. After 10 min water was added and the reaction mixture was extracted with 3×600 mL of DCM. The organic extracts were combined, dried over anhydrous sodium sulfate and concentrated under reduced pressure. The product was purified by silica gel chromatography (eluent: PE/EtOAc from 10/1 to 3/1). This resulted in 70.5 g (steps A and B, 78%) of 3-benzoyl-1-methylpyrrolidin-2-one-4,4,5,5-d₄ as a yellow oil. LC-MS (ESI): Mass calcd. for Cl₂H₉D₄NO₂ 207.1 m/z found 208 [M+H]⁺.

Step C: 3-Benzoyl-3-ethynyl-1-methylpyrrolidin-2-one-4,4,5,5-d₄. Into a 2000 mL, 4-necked round-bottom flask with a mechanical agitator, purged and maintained with an atmosphere of nitrogen, was placed 3-benzoyl-1-methylpyrrolidin-2-one-4,4,5,5-d₄ (70.5 g, 340 mmol) and THF (dry, 1200 mL). The resulting mixture was cooled to −78° C., and 1-((trimethylsilyl)ethynyl)-1λ3-benzo[d][1,2]iodaoxol-3(1H)-one (175 g, 510 mmol) was added in one portion and the reaction mixture was stirred at −78° C. for 10 min. TBAF (1 M in THF, 510 mL, 510 mmol) was added dropwise at −78° C. The resulting solution was stirred at −70° C. for 2 hours, and then warmed to room temperature for another 2 hours. The resulting mixture was quenched with saturated aqueous NH₄Cl solution, extracted with 3×600 mL of EtOAc and the organic extracts combined, dried over anhydrous sodium sulfate, filtered and concentrated under reduced pressure. The product was purified by silica gel chromatography (PE/EtOAc from 10/1 to 5/1) to afford 53.8 g (62%) of 3-benzoyl-3-ethynyl-1-methylpyrrolidin-2-one-4,4,5,5-d₄ as a yellow oil. LC-MS (ESI): Mass calcd. for C₁₄H₉D₄NO₂ 231.1 m/z found 232 [M+H]⁺.

Step D: 3-Ethynyl-1-methyl-2-oxopyrrolidin-3-yl-4,4,5,5-d₄ benzoate. Into a 5000-mL 4-necked round-bottom flask with mechanical agitator, was placed 3-benzoyl-3-ethynyl-1-methylpyrrolidin-2-one-4,4,5,5-d₄ (53.8 g, 233 mmol), DCM (3000 mL), KHCO₃ (116 g, 1165 mmol), and m-CPBA (236 g, 1165 mmol). The resulting mixture was stirred at room temperature overnight. The reaction mixture was then quenched with saturated aqueous NaHCO₃ solution, stirred for 1 hour, extracted with 2×1000 mL of DCM and the organic extracts combined, dried over anhydrous sodium sulfate and concentrated under reduced pressure. The product was purified by silica gel chromatography (PE/EtOAc from 10/1 to 3/1) to provide 27 g (47%) of 3-ethynyl-1-methyl-2-oxopyrrolidin-3-yl-4,4,5,5-d₄ benzoate as a yellow solid. LC-MS (ESI): Mass calcd. for C₁₄H₉D₄NO₃ 247.1 m/z found 248 [M+H]⁺.

Step E: 3-Ethynyl-3-hydroxy-1-methylpyrrolidin-2-one-4,4,5,5-d₄. Into a 1000-mL, 4-necked round-bottom flask, was placed 3-ethynyl-1-methyl-2-oxopyrrolidin-3-yl-4,4,5,5-d₄ benzoate (27 g, 109 mmol) and THF (270 mL) and the mixture was cooled to 0° C. A solution of LiOH—H₂O (13.8 g, 327 mmol) in H₂O (270 mL) at 0° C.-10° C. was added and the reaction mixture was stirred at room temperature for 5 hours. The mixture was then cooled to 0° C., the pH was adjusted to pH=7 with 1 M aqueous HCl, and the reaction mixture was concentrated under reduced pressure. The product was purified by silica gel chromatography (DCM/MeOH from 300/1 to 100/1) to afford 15.1 g (87%) of 3-ethynyl-3-hydroxy-1-methylpyrrolidin-2-one-4,4,5,5-d₄ as a yellow solid. LC-MS (ESI): Mass calcd. for C₇H₅D₄NO₂ 143.1 m/z found 144 [M+H]⁺.

Step F: (R)-3-Ethynyl-3-hydroxy-1-methylpyrrolidin-2-one-4,4,5,5-d₄. The (R) and (S) enantiomers of 3-ethynyl-3-hydroxy-1-methylpyrrolidin-2-one-4,4,5,5-d₄ were separated with SFC (Column: CHIRALPAK® IH, 3×25 cm, 5 μm; Mobile Phase A:CO₂, Mobile Phase B:IPA (0.5% 2 M NH₃-MeOH); Flow rate:100 mL/min; Gradient:10% B; Column Temperature: 35° C.; Back Pressure: 100 bar; 220 nm; RT1:3.62; RT2:4.82). The first eluting peak was (S)-3-ethynyl-3-hydroxy-1-methylpyrrolidin-2-one-4,4,5,5-d₄ and the second eluting peak was (R)-3-ethynyl-3-hydroxy-1-methylpyrrolidin-2-one-4,4,5,5-d₄. (R)-3-Ethynyl-3-hydroxy-1-methylpyrrolidin-2-one-4,4,5,5-d₄ was then slurried with MTBE (60 mL) and the solids were filtered off and collected. The filter cake was washed with MTBE and the filtrate was concentrated to afford 6.46 g (79%) of (R)-3-ethynyl-3-hydroxy-1-methylpyrrolidin-2-one-4,4,5,5-d₄ as white solid. LC-MS (ESI): Mass calcd. for C₇H₅D₄NO₂ 143.1 m/z found 144 [M+H]⁺.

### Intermediate 47: (R)-3-hydroxy-1-methyl-3-(3-(3-(4,4,5,5-tetramethyl-1,3,2-dioxaborolan-2-yl)phenyl)isoxazol-5-yl)pyrrolidin-2-one-4,4,5,5-d₄

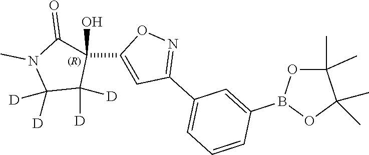

Step A. (R)-3-(3-(3-Bromophenyl)isoxazol-5-yl)-3-hydroxy-1-methylpyrrolidin-2-one-4,4,5,5-d₄. To a microwave vial containing (R)-3-ethynyl-3-hydroxy-1-methylpyrrolidin-2-one-4,4,5,5-d₄ (Intermediate 46, 250 mg, 1.75 mmol) and (Z)-3-bromo-N-hydroxybenzimidoyl chloride (409 mg, 1.75 mmol) was added DCM (11 mL) followed by Et₃N (0.73 mL). The vial was sealed, and the resulting homogeneous mixture was stirred at room temperature. After ˜10 min, a white suspension resulted and after 3.5 h, the reaction was judged to be complete by TLC. The contents were filtered through a pad of diatomaceous earth and rinsed with DCM. The colorless eluent was concentrated to give the product as an off-white solid. The material was redissolved in CHCl₃-MeOH and diatomaceous earth (3 g) was added and concentrated to dryness. The material was purified by FCC (using 100% DCM increasing to 5% MeOH-DCM) to give the title compound (405 mg, 67%) as a yellowish solid after drying. MS (ESI): Mass calcd. for C₁₄H₁₃BrN₂O₃ 341.2 m/z found 342.2 [M+H]⁺. ¹H NMR (400 MHz, Chloroform-d) δ 7.87 (t, J=1.8 Hz, 1H), 7.63 (dt, J=7.9, 1.3 Hz, 1H), 7.54 (ddd, J=8.0, 2.0, 1.0 Hz, 1H), 7.29 (d, J=7.9 Hz, 1H), 6.64 (s, 1H), 4.25 (s, 1H), 2.99 (s, 3H).

Step B. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one-4,4,5,5-d₄. To a microwave vial was added (R)-3-(3-(3-bromophenyl)isoxazol-5-yl)-3-hydroxy-1-methylpyrrolidin-2-one-4,4,5,5-d₄ (400 mg, 1.17 mmol), bis(pinacolato)diboron (416.8 mg, 1.64 mmol), chloro(2-dicyclohexylphosphino-2′,4′,6′-triisopropyl-1,1′-biphenyl)[2-(2′-amino-1,1′-biphenyl)]palladium(II) (92.2 mg, 0.12 mmol), KOAc (460 mg, 4.69 mmol) and 1,4-dioxane (21 mL, degassed with nitrogen for 20 min prior to use). The vial was sealed and evacuated/purged with nitrogen and then placed in an aluminum heating mantle at 110° C. After 2.5 h, the reaction mixture was filtered through a pad of diatomaceous earth (while still warm) and rinsed with EtOAc and THF and the effluent was concentrated to give an orange viscous oil. The material was dissolved in EtOAc and purified by FCC (using 100% hexanes increasing to 100% EtOAc) to give the titled compound as an off-white solid (223 mg, 49%). MS (ESI): Mass calcd. for C₂₀H₂₅BN₂O₅ 388.3 m/z found 339.3 [M+H]⁺. ¹H NMR (400 MHz, Chloroform-d) δ 8.15 (t, J=1.5 Hz, 1H), 7.89 (ddt, J=18.6, 7.4, 1.4 Hz, 2H), 7.44 (t, J=7.6 Hz, 1H), 6.74 (s, 1H), 3.72 (d, J=12.3 Hz, 1H), 2.99 (s, 3H), 1.36 (s, 12H).

### Intermediate 48: (3R,5S)-3-Hydroxy-1-methyl-3-(3-(3-(4,4,5,5-tetramethyl-1,3,2-dioxaborolan-2-yl)phenyl)isoxazol-5-yl)-5-(trifluoromethyl)pyrrolidin-2-one

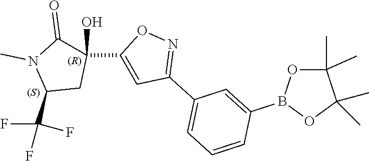

Step A. (3R,5S)-3-(3-(3-Bromophenyl)isoxazol-5-yl)-3-hydroxy-1-methyl-5-(trifluoromethyl)pyrrolidin-2-one. To a 50 mL flask containing (3R,5S)-3-ethynyl-3-hydroxy-1-methyl-5-(trifluoromethyl)pyrrolidin-2-one (Intermediate 45, 500 mg, 2.41 mmol) and (Z)-3-bromo-N-hydroxybenzimidoyl chloride (566 mg, 2.41 mmol) was added DCM (15 mL) followed by Et₃N (1 mL). The flask kept under nitrogen and the resulting homogeneous mixture was stirred at room temperature. After 24 h, the reaction was judged to be complete by TLC. The contents were filtered through a pad of diatomaceous earth and rinsed with DCM. The colorless eluent was concentrated to give the product as an off-white solid. The product was dissolved in CHCl₃-MeOH with diatomaceous earth (3 g), concentrated and purified by FCC (100% hexanes increasing to 100% EtOAc) to give the titled compound (815 mg, 83.0%) as a an off-white amorphous solid after drying. MS (ESI): Mass calcd. for C₁₅H₁₂BrF₃N₂O₃ 405.2 m/z found 406.0 [M+H]⁺. ¹H NMR (500 MHz, Chloroform-d) δ 7.88 (t, J=1.8 Hz, 1H), 7.65 (ddd, J=7.8, 1.6, 1.0 Hz, 1H), 7.57 (ddd, J=8.1, 2.0, 1.0 Hz, 1H), 7.30 (t, J=7.9 Hz, 1H), 6.70 (s, 1H), 4.35-4.20 (m, 1H), 4.10-3.98 (m, 1H), 3.09 (q, J=1.2 Hz, 3H), 3.06-2.93 (m, 2H), 2.48 (dd, J=14.2, 6.8 Hz, 1H).

Step B. (3R,5S)-3-Hydroxy-1-methyl-3-(3-(3-(4,4,5,5-tetramethyl-1,3,2-dioxaborolan-2-yl)phenyl)isoxazol-5-yl)-5-(trifluoromethyl)pyrrolidin-2-one. To a microwave vial was added (3R,5S)-3-(3-(3-bromophenyl)isoxazol-5-yl)-3-hydroxy-1-methyl-5-(trifluoromethyl)pyrrolidin-2-one (400 mg, 0.99 mmol), bis(pinacolato)diboron (351 mg, 1.38 mmol), chloro(2-dicyclohexylphosphino-2′,4′,6′-triisopropyl-1,1′-biphenyl)[2-(2′-amino-1,1′-biphenyl)]palladium(II) (77 mg, 0.099 mmol), KOAc (387.5 mg, 3.95 mmol) and 1,4-dioxane (18 mL, degassed with nitrogen for 20 min prior to use). The vial was sealed and evacuated/purged with nitrogen and then placed in an aluminum heating mantle at 110° C. After 4 h, The reaction mixture was filtered through a pad of diatomaceous earth (while still warm) and rinsed with EtOAc and THF and the mixture was concentrated to give a brownish viscous oil. The material was dissolved in EtOAc and purified by FCC (using 100% hexanes increasing to 100% EtOAc) to give the title compound (404 mg, 90.0%) initially as a viscous amber gum which solidifies to an amorphous solid under vacuum but was not completely pure by HPLC analysis and was used as is. MS (ESI): Mass calcd. for C₂₁H₂₄BF₃N₂O₅ 452.2 m/z found 453.3 [M+H]⁺.

### Intermediate 49: (R)-3-Ethynyl-3-hydroxy-1-(methyl-d₃)pyrrolidin-2-one

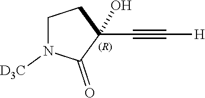

Step A: 4-((tert-Butoxycarbonyl)amino)-2-hydroxybutanoic acid. Into a 5 L 3-necked round-bottomed flask, purged and maintained with an inert atmosphere of nitrogen, was placed a solution of 4-amino-2-hydroxybutanoic acid (200 g, 1.67 mol) in water (1 L). This was followed by the addition of K₂CO₃ (695 g, 4.99 mol) in several batches at 0° C. To this mixture was added a solution of di-tert-butyl dicarbonate (436 g, 2 mol) in dioxane (1 L) dropwise with stirring at 0° C. The resulting solution was stirred for 24 h at 20-25° C. The resulting mixture was washed with petroleum ether (1 L×2). The combined aqueous phase was cooled to 0° C. with a water/ice bath and adjusted to pH=4-5 with aqueous HCl (6 N). The resulting solution was extracted with ethyl acetate (1 L×4). The combined organic layers were dried over anhydrous sodium sulfate, filtered, and concentrated to dryness to afford 4-((tert-butoxycarbonyl)amino)-2-hydroxybutanoic acid (260 g, 71%) as a yellow oil.

Step B: Methyl 4-((tert-butoxycarbonyl)amino)-2-hydroxybutanoate. Into a 5 L 3-necked round-bottomed flask, purged and maintained with an inert atmosphere of nitrogen, was placed a solution of 4-[[(tert-butoxy)carbonyl]amino]-2-hydroxybutanoic acid (260 g, 1.19 mol) in N,N-dimethylformamide (2.5 L) and Cs₂CO₃ (503 g, 1.54 mol). After 10 min, iodomethane (202 g, 1.42 mol) was added dropwise to the mixture with stirring at rt. After 4.5 h, the mixture was poured into water/ice (2 L) and extracted with ethyl acetate (2 L×2). The combined organic extracts were washed with brine (1 L×2), dried over anhydrous sodium sulfate, filtered, and concentrated to dryness. This afforded methyl 4-((tert-butoxycarbonyl)amino)-2-hydroxybutanoate (180 g, 65%) as a yellow oil.

Step C: Methyl 4-((tert-butoxycarbonyl)amino)-2-((tert-butyldimethylsilyl)oxy)butanoate. Into a 5 L 3-necked round-bottomed flask, purged and maintained with an inert atmosphere of nitrogen, was placed a solution of methyl 4-[[(tert-butoxy)carbonyl]amino]-2-hydroxybutanoate (180 g, 0.77 mol) in dichloromethane (1.8 L) and imidazole (108 g, 1.54 mol). This was followed by the addition of tert-butyl(chloro)dimethylsilane (231 g, 1.53 mol) in several batches at 0° C. The resulting solution was warmed to rt and stirred for 16 h. After which time, the mixture was poured into water/ice (1 L) and extracted with dichloromethane (1.5 L×3). The combined organic extracts were washed with brine (1 L), dried over anhydrous sodium sulfate, filtered, and concentrated to dryness. The resulting residue was purified by FCC (1:10, ethyl acetate/petroleum ether) to afford methyl 4-((tert-butoxycarbonyl)amino)-2-((tert-butyldimethylsilyl)oxy)butanoate (200 g, 75%) as a light yellow oil.

Step D: Methyl 4-((tert-butoxycarbonyl)(methyl-d₃)amino)-2-((tert-butyldimethylsilyl)oxy)butanoate. Into a 1 L 3-necked round-bottomed flask, purged and maintained with an inert atmosphere of nitrogen, was placed methyl 4-[[(tert-butoxy)carbonyl]amino]-2-[(tert-butyldimethylsilyl)oxy]butanoate (50.0 g, 144 mmol), N,N-dimethylformamide (500 mL), and CD₃I (62.6 g, 432 mmol). The resulting solution was cooled to 0° C. and sodium hydride (8.60 g, 358 mmol, 60% in mineral oil) was added in several batches at 0° C. After 2 h at 0° C., the mixture was poured into saturated aqueous NH₄Cl (250 mL). The resulting mixture was extracted with ethyl acetate (500 mL×2). The combined organic extracts were washed with brine (500 mL), dried over anhydrous sodium sulfate, filtered, and concentrated to dryness. The above procedure (Step D) was repeated 3× and afforded methyl 4-((tert-butoxycarbonyl)(methyl-d₃)amino)-2-((tert-butyldimethylsilyl)oxy)butanoate (200 g, 95%) as a light yellow oil.

Step E: Methyl 4-((tert-butoxycarbonyl)(methyl-d₃)amino)-2-hydroxybutanoate.

Into a 3 L 3-necked round-bottomed flask, purged and maintained with an inert atmosphere of nitrogen, was placed methyl 4-((tert-butoxycarbonyl)(methyl-ds)amino)-2-((tert-butyldimethylsilyl)oxy)butanoate (200 g, 549 mmol), methanol (2 L), and amine hydrofluoride (204 g, 5.51 mol). The resulting solution was heated at 50° C. After 12 h, the resulting solution was cooled to rt, concentrated to dryness, and diluted with water (1 L). The resulting mixture was extracted with ethyl acetate (1 L×3). The combined organic extracts were washed with brine (1 L), dried over anhydrous sodium sulfate, filtered, and concentrated to dryness. This afforded methyl 4-((tert-butoxycarbonyl)(methyl-d₃)amino)-2-hydroxybutanoate (137 g) as a light yellow oil which was used directly in the next step without further purification.

Step F: Methyl 4-((tert-butoxycarbonyl)(methyl-d₃)amino)-2-oxobutanoate. A 3 L 3-necked round-bottomed flask, purged and maintained with an inert atmosphere of nitrogen, was charged with methyl 4-((tert-butoxycarbonyl)(methyl-d₃)amino)-2-hydroxybutanoate (137 g, 547 mmol), dichloromethane (1.4 L), and 1,1,1-tris(acetyloxy)-1,1-dihydro-1,2-benziodoxol-3-(1H)-one, (Dess-Martin periodinane, 348 g, 821 mmol) at 5° C. The resulting mixture was stirred for 3 h at rt. After which time the mixture was poured into aqueous sodium bicarbonate (2 L). The resulting solids were filtered off and filtrate was extracted with dichloromethane (1.5 L×3). The combined organic extracts were washed with brine (1 L), dried over anhydrous sodium sulfate, filtered, and concentrated to dryness. The resulting residue was purified by FCC (1:3, ethyl acetate/petroleum ether) to afford methyl 4-((tert-butoxycarbonyl)(methyl-d₃)amino)-2-oxobutanoate (81 g, 60%) as a yellow oil. ¹H NMR (300 MHz, CDCl₃) δ 3.90 (s, 3H), 3.56 (t, J=6.6 Hz, 2H), 3.08 (t, J=6.6 Hz, 2H), 1.48 (s, 9H).

Step G: Methyl 2-(2-((tert-butoxycarbonyl)(methyl-ds)amino)ethyl)-2-hydroxybut-3-ynoate. Into a 1 L 3-necked round-bottomed flask, purged and maintained with an inert atmosphere of nitrogen, was placed a solution of methyl 4-((tert-butoxycarbonyl)(methyl-d₃)amino)-2-oxobutanoate (20 g, 81 mmol) in THF (0.2 L). The solution was cooled to −78° C., followed by dropwise addition of bromo(ethynyl)magnesium (274 mL, 138 mmol). The resulting solution was stirred at −40° C. After 2 h, saturated aqueous NH₄Cl (100 mL) was added dropwise at −70° C. The resulting mixture was warmed slowly to rt and extracted with ethyl acetate (800 mL×3). The combined organic extracts were washed with brine (800 mL), dried over anhydrous sodium sulfate, filtered, and concentrated to dryness. The above procedure (Step G) was repeated 3× and the combined residues afforded methyl 2-(2-((tert-butoxycarbonyl)(methyl-ds)amino)ethyl)-2-hydroxybut-3-ynoate (82 g) as a yellow oil.

Step H: Methyl 2-hydroxy-2-(2-((methyl-d₃)amino)ethyl)but-3-ynoate as a trifluoroacetate salt. Into a 1 L 3-necked round-bottomed flask purged and maintained with an inert atmosphere of nitrogen, was placed a solution of methyl 2-(2-((tert-butoxycarbonyl)(methyl-ds)amino)ethyl)-2-hydroxybut-3-ynoate (70.0 g, 255 mmol), dichloromethane (420 mL), and trifluoroacetic acid (140 mL). The resulting solution was stirred for 1 h at rt. The resulting mixture was concentrated to dryness and used directly in the next step without further purification.

Step I: (R)-3-Ethynyl-3-hydroxy-1-(methyl-d₃)pyrrolidin-2-one. Into a 1 L 3-necked round-bottomed flask purged and maintained with an inert atmosphere of nitrogen, was placed a solution of methyl 2-hydroxy-2-(2-((methyl-d₃)amino)ethyl)but-3-ynoate as a trifluoroacetate salt (70.0 g, 243 mmol), methanol (700 mL), and potassium carbonate (133 g, 964 mmol). The resulting solution was stirred for 3 h at rt. The resulting solids were filtered off and the filtrate was concentrated to dryness. The resulting residue was purified by FCC (1:5, ethyl acetate/petroleum ether) and then recrystallized from diethyl ether (100 mL) to afford racemic 3-ethynyl-3-hydroxy-1-(methyl-d₃)pyrrolidin-2-one (16 g, 46%) as a yellow solid. This material was further purified by preparative chiral SFC (CHIRALPAK® AS-H, 5×25 cm, 5 m; mobile phase, CO₂ (80%) and IPA (0.1% DEA) (20%); Detector, UV at 25° C.=220 nm) to afford (R)-3-ethynyl-3-hydroxy-1-(methyl-d₃)pyrrolidin-2-one (5.4 g, 34%, >97% ee) as a brown solid and (S)-3-ethynyl-3-hydroxy-1-(methyl-d₃)pyrrolidin-2-one (5.2 g, 33%, >97% ee) as a brown solid. Data for (R)-3-ethynyl-3-hydroxy-1-(methyl-ds)pyrrolidin-2-one: MS (ESI): Mass calcd. for C₇H₆D₃NO₂, 142.1; m/z found, 143.2 [M+H]⁺. ¹H NMR (400 MHz, CD₃OD) δ 3.41-3.38 (t, J=5.2 Hz, 2H), 3.03 (s, 1H), 2.48-2.43 (m, 1H), 2.24-2.17 (m, 1H). Data for (S)-3-ethynyl-3-hydroxy-1-(methyl-d₃)pyrrolidin-2-one: MS (ESI): Mass calcd. for C₇H₆D₃NO₂, 142.08; m/z found, 143.2 [M+H]⁺. ¹H NMR (400 MHz, CD₃OD) δ 3.41-3.38 (t, J=5.2 Hz, 2H), 3.03 (s, 1H), 2.48-2.43 (m, 1H), 2.24-2.17 (m, 1H).

### Intermediate 50: (R)-3-Hydroxy-1-(methyl-d₃)-3-(3-(3-(4,4,5,5-tetramethyl-1,3,2-dioxaborolan-2-yl)phenyl)isoxazol-5-yl)pyrrolidin-2-one

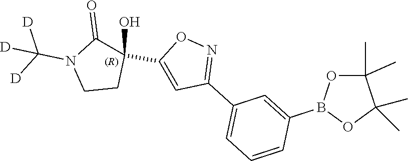

Step A. (R)-3-(3-(3-Bromophenyl)isoxazol-5-yl)-3-hydroxy-1-(methyl-d₃)pyrrolidin-2-one. To a vial containing (R)-3-ethynyl-3-hydroxy-1-(methyl-d₃)pyrrolidin-2-one (Intermediate 49, 500 mg, 3.52 mmol) and (Z)-3-bromo-N-hydroxybenzimidoyl chloride (800 mg, 3.41 mmol) was added DCM (22 mL) followed by Et₃N (1.5 mL). The flask was sealed, and the homogeneous mixture was stirred at room temp. After 24 h, the contents were filtered through a pad of diatomaceous earth and rinsed with DCM. The mixture was concentrated to give the product as a viscous orange gum. The product was dissolved in DCM (40 mL), water (20 mL) was added and the layers were separated. The aqueous portion was back-extracted 3 times with CHCl₃ (10 mL) and the combined organic extracts were dried over MgSO₄, filtered and concentrated to give an orange oil which was purified by FCC (100% DCM increasing to 5% MeOH-DCM) to give the title compound (810 mg, 67%) as a hygroscopic semi-solid MS (ESI): Mass calcd. for C₁₄H₁₃BrN₂O₃ 340.2 m/z found 341.7 [M+H]⁺. ¹H NMR (400 MHz, Chloroform-d) δ 7.87 (t, J=1.8 Hz, 1H), 7.69-7.47 (m, 2H), 7.27 (t, J=7.9 Hz, 2H), 6.67 (s, 1H), 4.66 (s, 1H), 3.76 (s, 1H), 3.60-3.67 (m, 1H), 3.57-3.27 (m, 2H), 2.72-2.77 (m, 1H), 2.59-2.38 (m, 2H), 2.25-2.33 (m, 1H).

Step B. (R)-3-Hydroxy-1-(methyl-d₃)-3-(3-(3-(4,4,5,5-tetramethyl-1,3,2-dioxaborolan-2-yl)phenyl)isoxazol-5-yl)pyrrolidin-2-one. To a 50 mL flask containing (R)-3-(3-(3-bromophenyl)isoxazol-5-yl)-3-hydroxy-1-(methyl-d₃)pyrrolidin-2-one (800 mg, 2.35 mmol) was added bis(pinacolato)diboron (836 mg, 3.29 mmol), chloro(2-dicyclohexylphosphino-2′,4′,6′-triisopropyl-1,1′-biphenyl)[2-(2′-amino-1,1′-biphenyl)]palladium(II) (185 mg, 0.24 mmol), KOAc (923 mg, 9.41 mmol) and 1,4-dioxane (42 mL, degassed with nitrogen for 20 min prior to use). The flask was fitted with a reflux condenser and evacuated/purged with nitrogen and then placed in an aluminum heating mantle at 105° C. After 4.5 h, TLC (20% EtOAc-DCM) indicated the reaction was complete. The mixture was filtered through a pad of diatomaceous earth (while still warm) and rinsed with EtOAc and THF and the filtrate was concentrated to a brownish viscous oil. The material was dissolved in EtOAc and purified by FCC (100% hexanes increasing to 100% EtOAc) to give the title product (211 mg, 23%) as an amber amorphous solid. MS (ESI): Mass calcd. for C₂₀H₂₅BN₂O₅ 387.3 m/z found 388.3 [M+H]⁺. ¹H NMR (400 MHz, Chloroform-d) δ 8.15 (d, J=1.6 Hz, 1H), 7.85-7.94 (m, 2H), 7.44 (t, J=7.6 Hz, 1H), 6.74 (s, 1H), 3.68-3.74 (m, 1H), 3.58-3.66 (m, 1H), 3.42-3.47 (m, 1H), 2.77-2.81 (m, 1H), 2.41-2.50 (m, 1H), 1.36 (s, 12H).

### Intermediate 51: 4-Bromo-6-chloropicolinamide

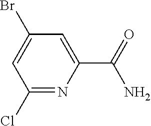 [SMILES: NC(=O)c1cc(Br)cc(Cl)n1]

To a 25 mL round-bottom flask containing 4-bromo-6-chloropicolinonitrile (360 mg, 1.7 mmol) was added acetone (8.3 mL), water (2.8 mL), and K₂CO₃ (114 mg, 0.8 mmol). To this stirring mixture was then added urea-hydrogen peroxide (1.6 g, 16.6 mmol) and the resulting mixture was stirred at room temperature. After 90 minutes, the resulting solution was diluted with water and extracted with ethyl acetate. The resulting organic solution was then dried over MgSO₄, filtered, and evaporated to dryness, to yield a white solid (320 mg, 82%) that was used without further purification. LC-MS (ESI): Mass calcd. for C₆H₄BrClN₂O 233.9 m/z found 234.9 [M+H]⁺.

### Intermediate 52: 6-Chloro-4-(1-methyl-1H-pyrazol-5-yl)picolinamide

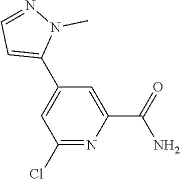 [SMILES: Cn1nccc1-c1cc(Cl)nc(C(N)=O)c1]

A mixture consisting of 4-bromo-6-chloropicolinamide (100 mg, 0.43 mmol), 1-methyl-5-(4,4,5,5-tetramethyl-1,3,2-dioxaborolan-2-yl)-1H-pyrazole (132 mg, 0.63 mmol), K₃PO₄ (276 mg, 1.27 mmol), 1,4-dioxane (1.1 mL), and water (0.2 mL) was subsequently evacuated and refilled with N₂ for 3 times, treated with Pd(dppf)Cl₂ (46 mg, 0.064 mmol), and heated at 100° C. for 3 h under N₂. The reaction vessel was removed from the heating mantle and allowed to gradually cool to rt. The resulting mixture was diluted with water and extracted with ethyl acetate. The resulting organic solution was then dried over MgSO₄, filtered, and evaporated to dryness. The residue was subjected to flash column chromatography, with a gradient of 0% to 10% MeOH in DCM over 15 minutes, to afford an orange oil (100 mg). MS (ESI): Mass calcd. for C₁₀H₉ClN₄O 236.1 m/z, found 237.0 [M+H]⁺.

### Intermediate 53: 6-chloro-4-(isothiazol-5-yl)picolinamide

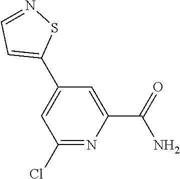 [SMILES: NC(=O)c1cc(-c2ccns2)cc(Cl)n1]

The title compound (60 mg, 59%) was prepared with analogous conditions to those described in Intermediate 52 using 5-(4,4,5,5-tetramethyl-1,3,2-dioxaborolan-2-yl)isothiazole in place of 1-methyl-5-(4,4,5,5-tetramethyl-1,3,2-dioxaborolan-2-yl)-1H-pyrazole. MS (ESI): Mass calcd. for C₉H₆ClN₃OS 239.0 m/z, found 240.0 [M+H]⁺.

### Intermediate 54: 6-chloro-4-(thiazol-5-yl)picolinamide

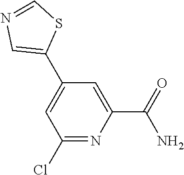 [SMILES: NC(=O)c1cc(-c2cncs2)cc(Cl)n1]

The title compound (60 mg, 59%) was prepared with analogous conditions to those described in Intermediate 52 using 5-(4,4,5,5-tetramethyl-1,3,2-dioxaborolan-2-yl)thiazole in place of 1-methyl-5-(4,4,5,5-tetramethyl-1,3,2-dioxaborolan-2-yl)-1H-pyrazole. MS (ESI): Mass calcd. for C₉H₆ClN₃OS 239.0 m/z, found 239.9 [M+H]⁺.

### Intermediate 55: (R)-4-chloro-6-(3-(5-(3-hydroxy-1-methyl-2-oxopyrrolidin-3-yl)isoxazol-3-yl)phenyl)picolinic acid

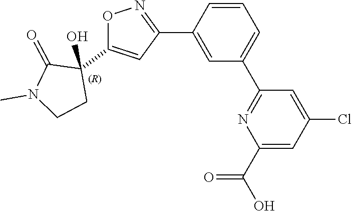

To a mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 10.0 g, 26.0 mmol) and methyl 4,6-dichloropicolinate (10.7 g, 52.1 mmol) in 1,4-dioxane (75.0 mL) and water (25.0 mL) were added K₂CO₃ (7.19 g, 52.1 mmol) and Pd(PPh₃)₄ (1.50 g, 1.30 mmol) and the resulting mixture was degassed and purged with N₂ for 3 times, and then the mixture was heated at 90° C. for 4 hr under N₂ atmosphere. The reaction mixture was diluted with water (40 mL), the pH of the mixture was adjusted to pH 3, and the mixture was extracted with ethyl acetate (50.0 mL×3). The combined organic layers were washed with brine (50.0 mL×1), dried over Na₂SO₄, filtered and concentrated under reduced pressure to give a residue. The product was triturated with ethyl acetate (15 mL) at 25° C. for 15 mins. The title compound was obtained as a white solid (8.50 g, 19.3 mmol, 74.2%). MS (ESI): Mass calcd. for C₂₀H₁₆ClN₃O₅ 413.1 m/z, found 414.1 [M+H]⁺.

### Intermediate 56: Methyl (R)-4-chloro-6-(3-(5-(3-hydroxy-1-methyl-2-oxopyrrolidin-3-yl)isoxazol-3-yl)phenyl)picolinate

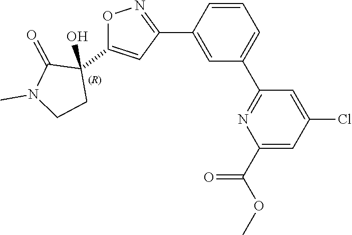

To a solution of (R)-4-chloro-6-(3-(5-(3-hydroxy-1-methyl-2-oxopyrrolidin-3-yl)isoxazol-3-yl)phenyl)picolinic acid (16.0 g, 38.7 mmol, Intermediate 55) in MeOH (35.0 mL) and THF (35.0 mL) was slowly added trimethylsilyl-diazomethane (2 M, 58.00 mL) at 0° C. under N₂. The mixture was stirred slowly warming from 0-25° C. over 2 hrs. The reaction mixture was filtered and concentrated under reduced pressure to give a residue. The residue was purified by flash column chromatography (petroleum ether: ethyl acetate=1:0 to 0:1) to yield product. The product was triturated with MTBE (60 mL at 25° C. for 48 hrs). Compound was obtained as a white solid (11.8 g, 26.3 mmol, 68.0%). MS (ESI): Mass calcd. for C₂₁H₁₈ClN₃O₅ 427.1 m/z, found 428.3 [M+H]⁺. ¹H NMR (400 MHz, DMSO-d₆) δ 8.53-8.66 (m, 2H), 8.31 (br d, J=7.88 Hz, 1H), 7.99-8.10 (m, 2H), 7.69 (t, J=7.75 Hz, 1H), 7.19 (s, 1H), 6.75 (s, 1H), 3.95 (s, 3H), 3.38-3.55 (m, 2H), 2.85 (s, 3H), 2.53-2.64 (m, 1H), 2.29 (ddd, J=13.38, 7.63, 6.00 Hz, 1H).

### Intermediate 57: (R)-4-Chloro-6-(3-(5-(3-hydroxy-1-methyl-2-oxopyrrolidin-3-yl)isoxazol-3-yl)phenyl)picolinamide

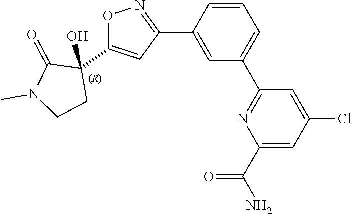

A solution of ammonia (11.7 mL, 2 M in MeOH) and methyl (R)-4-chloro-6-(3-(5-(3-hydroxy-1-methyl-2-oxopyrrolidin-3-yl)isoxazol-3-yl)phenyl)picolinate (Intermediate 56, 1 g, 2.3 mmol) was stirred in a microwave at 100° C. for 80 minutes. The resulting solution was evaporated to dryness to yield a white solid (1 g) which was used as-is. MS (ESI): Mass calcd. for C₂₀H₁₇ClN₄O₄ 412.1 m/z, found 413.1 [M+H]⁺.

### Intermediate 58: 6,8-Dichloropyrido[3,2-d]pyrimidin-4-amine

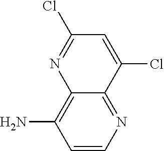 [SMILES: Nc1ccnc2c(Cl)cc(Cl)nc12]

A flask was charged with a solution of 3-amino-4,6-dichloropicolinonitrile (1.21 g, 6.34 mmol), K₃PO₄ (13.6 g, 64.0 mmol), and 1,4-dioxane (50 mL) followed by formimidamide acetate (3.87 g, 37.1 mmol). The resulting mixture was heated at 100° C. for 16 h. The resulting mixture was cooled to rt and concentrated to dryness. The residue was diluted with H₂O (50 mL) and stirred at rt for 16 h. The resulting mixture was filtered, the filter cake was washed with water (200 mL), and the solid was collected. The resulting solid was added to DCM (50 mL) and the mixture was stirred at rt for 40 min. The resulting solids were collected by filtration and dried to afford 6,8-dichloropyrido[3,2-d]pyrimidin-4-amine (1.35 g, 97.5%) as a white solid. MS (ESI): Mass calcd. for C₇H₄Cl₂N₄, 214.0; m/z found, 215.0 [M+H]⁺. ¹H NMR (500 MHz, CDCl₃) δ 8.72 (s, 1H), 7.81 (s, 1H), 5.95-5.48 (m, 2H).

### Intermediate 59: 6-Chloropyrido[3,2-d]pyrimidin-4-amine

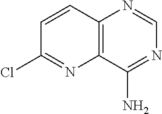 [SMILES: Nc1ncnc2ccc(Cl)nc12]

Formimidamide acetate (25.0 g, 240 mmol) was added into a solution of 3-amino-6-chloropicolinonitrile (8.80 g, 57.3 mmol) and K₃PO₄ (78.0 g, 367 mmol) in 1,4-dioxane (400 mL). The reaction was heated at 100° C. for 2 h. After the reaction mixture was cooled to room temperature, the reaction mixture was concentrated to dryness in vacuo to give the product, which was poured into H₂O (500 mL), and stirred at room-temperature for 16 hours. The suspension was filtered. The filter cake was washed with H₂O (100 mL) and the filtrate was poured into CHCl₃ (70 mL). The mixture was heated at 50° C. for 30 min. The suspension was isolated via filtration before cooling to room-temperature, and the filter cake was washed with CHCl₃ (30 mL). Then the filter cake was dried in vacuo to afford the product (7.8 g, 75%) as a yellow solid. ¹H NMR (400 MHz, DMSO-d₆) δ 8.41 (s, 1H), 8.10 (d, J=8.8 Hz, 1H), 8.04-7.90 (m, 2H), 7.84 (d, J=8.6 Hz, 1H).

### Intermediate 60: tert-Butyl 3-((hydroxyimino)methyl)piperidine-1-carboxylate

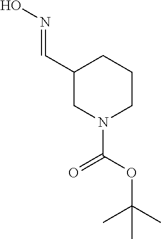 [SMILES: CC(C)(C)OC(=O)N1CCCC(/C=N/O)C1]

A mixture of tert-butyl 3-formylpiperidine-1-carboxylate (3.50 g, 16.4 mmol), hydroxylamine hydrochloride (1.71 g, 24.6 mmol) and Na₂CO₃ (2.61 g, 24.6 mmol) in EtOH (50 mL) and water (5 mL) was heated to 80° C. for 16 h. The reaction vessel was removed from the oil bath and allowed to gradually cool to room temperature. The mixture was concentrated in vacuo, diluted with water (40 mL), and extracted with EtOAc (100 mL×3). The combined organic layers were dried over anhydrous Na₂SO₄, filtered, and concentrated in vacuo to give tert-butyl 3-((hydroxyimino)methyl)piperidine-1-carboxylate as a solid (3.2 g), which was used directly in the next step without purification. ¹H NMR (400 MHz, CDCl₃) δ 7.36 (d, J=5.5 Hz, 1H), 4.12-3.70 (m, 3H), 2.90-2.79 (m, 2H), 2.44-2.33 (m, 1H), 1.98-1.76 (m, 2H), 1.46 (s, 9H). ¹H NMR (400 MHz, MeOD) δ 7.26 (d, J=5.7 Hz, 1H), 4.01-3.64 (m, 3H), 3.06-2.87 (m, 2H), 2.34-2.24 (m, 1H), 1.91-1.81 (m, 1H), 1.75-1.62 (m, 2H), 1.43 (s, 9H).

### Intermediate 61: tert-Butyl 3-(chloro(hydroxyimino)methyl)piperidine-1-carboxylate

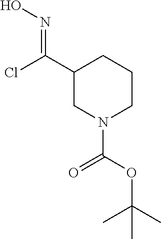 [SMILES: CC(C)(C)OC(=O)N1CCCC(/C(Cl)=N/O)C1]

NCS (1.87 g, 14.0 mmol) was added to a solution consisting of tert-butyl 3-((hydroxyimino)methyl)piperidine-1-carboxylate (Intermediate 60, 3.2 g, 14 mmol) and N,N-dimethylformamide (100 mL) and the resulting mixture was stirred for 16 h at room temperature under nitrogen. After this time, the mixture was diluted with ethyl acetate (200 mL) and washed with water (50 mL×3). The organic layer was dried over anhydrous Na₂SO₄, filtered, and concentrated to dryness in vacuo. The residue was subjected to silica gel chromatography (0-30% EtOAc/petroleum ether) to afford tert-butyl 3-(chloro(hydroxyimino)methyl)piperidine-1-carboxylate as a colorless oil (1.36 g, 29%). MS (ESI): Mass calcd. for C₁H₁₉ClN₂O₃ 262.1 m/z found 207.1 [M-55]⁺.

### Intermediate 62: tert-Butyl 3-(5-((R)-3-hydroxy-1-methyl-2-oxopyrrolidin-3-yl)isoxazol-3-yl)piperidine-1-carboxylate

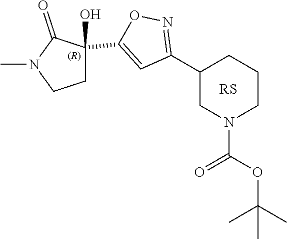

(R)-3-Ethynyl-3-hydroxy-1-methylpyrrolidin-2-one (Intermediate 1, 500 mg, 3.59 mmol), tert-butyl 3-(chloro(hydroxyimino)methyl)piperidine-1-carboxylate (Intermediate 61, 1.3 g, 4.9 mmol), and NaHCO₃ (604 mg, 7.19 mmol) in EtOAc (20 mL) was stirred at room temperature for 16 h. After this time, the mixture was concentrated to dryness in vacuo. The residue was subjected to silica gel chromatography (10-100% EtOAc/petroleum ether) to afford tert-butyl 3-(5-((R)-3-hydroxy-1-methyl-2-oxopyrrolidin-3-yl)isoxazol-3-yl)piperidine-1-carboxylate (1.32 g, 99.8%) as a white solid. MS (ESI): Mass calcd. for C₁₈H₂₇N₃O₅ 365.2 m/z found 266.2 [M-99]⁺.

### Intermediate 63: (3R)-3-Hydroxy-1-methyl-3-(3-(piperidin-3-yl)isoxazol-5-yl)pyrrolidin-2-one

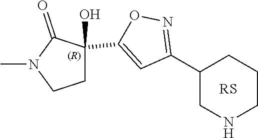

TFA (3.6 mL, 48 mmol) was added to a solution of tert-butyl 3-(5-((R)-3-hydroxy-1-methyl-2-oxopyrrolidin-3-yl)isoxazol-3-yl)piperidine-1-carboxylate (Intermediate 62, 1.2 g, 3.3 mmol) in DCM (36 mL) at room temperature. The resulting mixture was stirred for 2 h at room temperature. After this time, the mixture was treated with sat. aqueous NaHCO₃ (50 mL) to pH 8-9 and lyophilized to dryness. The residue was triturated with DCM/EtOAc/MeOH (200 mL, 10:10:1), filtered, and the filtrate concentrated to dryness in vacuo to afford (3R)-3-hydroxy-1-methyl-3-(3-(piperidin-3-yl)isoxazol-5-yl)pyrrolidin-2-one as a sticky solid (4 g). The product was used for next step directly without purification. MS (ESI): Mass calcd. for C₁₃H₁₉N₃O₃ 265.1 m/z found 266.0 [M+1]⁺.

### Intermediate 64: Ethyl 3-bromo-5,6,7,8-tetrahydroimidazo[1,5-a]pyrazine-1-carboxylate

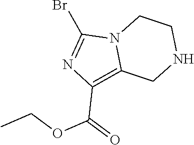

Ethyl 5,6,7,8-tetrahydroimidazo[1,5-a]pyrazine-1-carboxylate hydrochloride (1.0 g, 4.3 mmol) and MeCN (10 mL) were added to a 40 mL flask, and charged with NBS (768 mg, 4.32 mmol). The resulting mixture stirred for 2 h at room temperature, quenched with sat. aq. Na₂S₂O₃ (20 mL) and stirred for 15 min at room temperature. After this time, the mixture was basified with sat. aq. NaHCO₃ to pH=8 and concentrated to dryness in vacuo to give a yellow solid. The yellow solid was then subjected to silica gel chromatography (0-10% MeOH/DCM) to give ethyl 3-bromo-5,6,7,8-tetrahydroimidazo[1,5-a]pyrazine-1-carboxylate as a yellow solid (290 mg, 21%). LCMS (ESI): Mass calcd. for C₉H₁₂BrN₃O₂ 273.0 m/z, found 276.0 [M+H]⁺.

### Intermediate 65: (R)-3-(3-(2-chloropyridin-4-yl)isoxazol-5-yl)-3-hydroxy-1-methylpyrrolidin-2-one

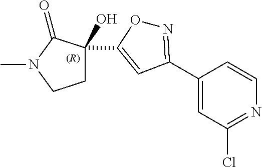

(R)-3-(3-(2-Chloropyridin-4-yl)isoxazol-5-yl)-3-hydroxy-1-methylpyrrolidin-2-one (512 mg) was prepared in a manner analogous to Intermediate 7: (R)-3-(3-(2-Bromopyridin-4-yl)isoxazol-5-yl)-3-hydroxy-1-methylpyrrolidin-2-one. using 2-chloroisonicotinaldehyde instead of 2-bromoisonicotinaldehyde. MS (ESI): Mass calcd. for C₁₃H₁₂ClN₃O₃, 293.1; m/z found, 293.9 [M+H]⁺.

### Example 1: (R)-3-(3-(3-(4-Amino-2-methylpyrido[3,2-d]pyrimidin-6-yl)phenyl)isoxazol-5-yl)-3-hydroxy-1-methylpyrrolidin-2-one

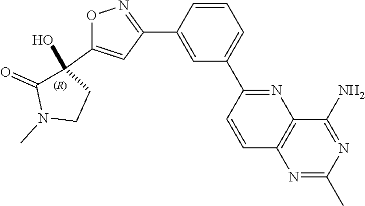

To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 150 mg, 0.39 mmol) in 1,4-dioxane (8 mL) and H₂O (1 mL) was added 6-chloro-2-methylpyrido[3,2-d]pyrimidin-4-amine (Intermediate 16, 152 mg, 0.780 mmol), followed by bis(tri-tert-butylphosphine)palladium(0) (20 mg, 0.04 mmol) and KF (68 mg, 1.2 mmol). The mixture was heated at 85° C. for 3 h, under a N₂ atmosphere, cooled to rt and then concentrated. The resulting residue was purified by reverse phase HPLC using an HPLC column, such as an Xtimate C18 10 μm, 21.2×250 mm column using a 5% to 38% gradient of CH₃CN/H₂O (10 mM NH₄HCO₃ and 0.025% NH₄OH) to afford (R)-3-(3-(3-(4-amino-2-methylpyrido[3,2-d]pyrimidin-6-yl)phenyl)isoxazol-5-yl)-3-hydroxy-1-methylpyrrolidin-2-one (5.9 mg, 4%) as a white solid. MS (ESI): Mass calcd. for C₂₂H₂ON₆O₃, 416.4; m/z found, 417.2 [M+H]⁺. ¹H NMR (400 MHz, DMSO-d₆) δ 8.75 (s, 1H), 8.52-8.56 (m, 2H), 8.30 (s, 1H), 8.13 (s, 1H), 8.08 (d, J=8.0 Hz, 1H), 8.01 (d, J=8.0 Hz, 1H), 7.93 (s, 1H), 7.67 (t, J=7.6 Hz, 1H), 7.32 (s, 1H), 3.41-3.53 (m, 2H), 2.85 (s, 3H), 2.55-2.61 (m, 1H), 2.47 (s, 3H), 2.26-2.33 (m, 1H).

### Example 2: (R)-3-(3-(3-(4-Aminopyrido[3,2-d]pyrimidin-6-yl-2-d)phenyl)isoxazol-5-yl)-3-hydroxy-1-methylpyrrolidin-2-one

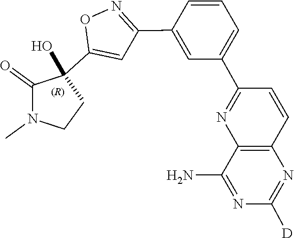

To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 15 mg, 0.39 mmol) in 1,2-dioxane (2 mL) and H₂O (0.2 mL) was added 6-chloropyrido[3,2-d]pyrimidin-2-d-4-amine (Intermediate 17, 71 mg, 0.39 mmol), followed by bis(tri-tert-butylphosphine)palladium(0) (20 mg, 0.04 mmol) and K₃PO₄ (249 mg, 1.17 mmol). The mixture was heated at 90° C. for 16 h under a N₂ atmosphere, cooled to rt, and concentrated under reduced pressure. The residue was purified by reverse phase HPLC using an HPLC column, such as an Xtimate 10 μm, 150 Å, 21.2×250 mm column using a 20 to 35% gradient of CH₃CN/H₂O (10 mM NH₄HCO₃ and 0.025% NH₄OH) to afford (R)-3-(3-(3-(4-aminopyrido[3,2-d]pyrimidin-6-yl-2-d)phenyl)isoxazol-5-yl)-3-hydroxy-1-methylpyrrolidin-2-one (28 mg, 18%) as a brown solid. MS (ESI): Mass calcd. for C₂₁H₁₇DN₆O₃, 403.1; m/z found, 404.0 [M+H]⁺. ¹H NMR (400 MHz, DMSO-d₆) δ 8.78 (s, 1H), 8.59 (d, J=7.2 Hz, 2H), 8.28 (s, 1H), 8.17 (d, J=8.8 Hz, 1H), 8.00-8.10 (m, 2H), 7.68 (t, J=8.0 Hz, 1H), 7.34 (s, 1H), 6.78 (s, 1H), 3.40-3.60 (m, 2H), 2.86 (s, 3H), 2.50-2.60 (m, 1H), 2.20-2.40 (m, 1H).

### Example 3: (R)-3-(5-(3-(4-Amino-2-methylpyrido[3,2-d]pyrimidin-6-yl)phenyl)isoxazol-3-yl)-3-hydroxy-1-methylpyrrolidin-2-one

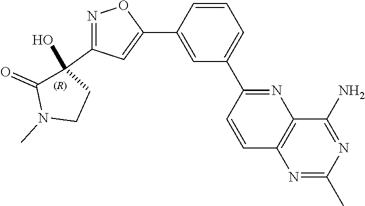

(R)-3-(5-(3-(4-Amino-2-methylpyrido[3,2-d]pyrimidin-6-yl)phenyl)isoxazol-3-yl)-3-hydroxy-1-methylpyrrolidin-2-one (57.8 mg, 53%) was prepared using analogous conditions to those described in Example 1 using (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₂H₂₀N₆O₃, 416.4; m/z found, 417.2 [M+H]⁺. ¹H NMR (400 MHz, DMSO-d₆) δ 8.83 (s, 1H), 8.53-8.56 (m, 2H), 8.17 (s, 1H), 8.08 (d, J=8.8 Hz, 1H), 7.94-7.97 (m, 2H), 7.69 (t, J=8.0 Hz, 1H), 7.39 (s, 1H), 6.52 (s, 1H), 3.37-3.49 (m, 2H), 2.85 (s, 3H), 2.63-2.71 (m, 1H), 2.47 (s, 3H), 2.22-2.29 (m, 1H).

### Example 4: (R)-3-(3-(3-(3-Amino-1H-pyrazolo[4,3-b]pyridin-5-yl)phenyl)isoxazol-5-yl)-3-hydroxy-1-methylpyrrolidin-2-one

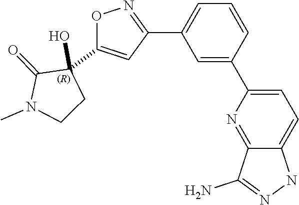

Step A: (R)-3-Hydroxy-1-methyl-3-(3-(3-(3-nitro-1H-pyrazolo[4,3-b]pyridin-5-yl)phenyl)isoxazol-5-yl)pyrrolidin-2-one. To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 50 mg, 0.1 mmol) in dioxane (2 mL) and DMF (1 mL) was added 5-chloro-3-nitro-1H-pyrazolo[4,3-b]pyridine (26 mg, 0.13 mmol), followed by Pd(t-Bu₃P)₂ (7.0 mg, 0.013 mmol) and K₃PO₄ (83 mg, 0.39 mmol). The mixture was heated at 95° C. for 16 h under a N₂ atmosphere, then cooled to rt and diluted with water (20 mL). The resulting aqueous mixture was extracted with DCM (4×15 mL). The combined organic solvent extracts were concentrated and purified using preparative TLC (DCM/MeOH=20/1) to afford (R)-3-hydroxy-1-methyl-3-(3-(3-(3-nitro-1H-pyrazolo[4,3-b]pyridin-5-yl)phenyl)isoxazol-5-yl)pyrrolidin-2-one (8 mg, 15%) as a brown solid. MS (ESI): Mass calcd. for C₂₀H₁₆N₆O₅, 420.4; m/z found, 421.0 [M+H]⁺.

Step B: (R)-3-(3-(3-(3-Amino-1H-pyrazolo[4,3-b]pyridin-5-yl)phenyl)isoxazol-5-yl)-3-hydroxy-1-methylpyrrolidin-2-one. To a solution of (R)-3-hydroxy-1-methyl-3-(3-(3-(3-nitro-1H-pyrazolo[4,3-b]pyridin-5-yl)phenyl)isoxazol-5-yl)pyrrolidin-2-one (15 mg, 0.036 mmol) in ethanol (2 mL) and 1,2-dichloroethane (1 mL) was added a porous nickel-aluminum alloy (nickel aluminide) such as Raney Ni (50 mg), followed by hydrazine hydrate (71 mg, 1.4 mmol). The mixture was stirred at 20° C. for 1 h and then filtered and concentrated. The resulting residue was purified by reverse phase HPLC using an HPLC column, such as an Xtimate C18 10 μm, 21.2×250 mm column using a 5 to 35% gradient of CH₃CN/H₂O (10 mM NH₄HCO₃ and 0.025% NH₄OH) to afford (R)-3-(3-(3-(3-amino-1H-pyrazolo[4,3-b]pyridin-5-yl)phenyl)isoxazol-5-yl)-3-hydroxy-1-methylpyrrolidin-2-one (5 mg, 36%) as an off-white solid. MS (ESI): Mass calcd. for C₂₀H₁₈N₆O₃, 390.4; m/z found, 391.0 [M+H]⁺. ¹H NMR (400 MHz, DMSO-d₆) δ 11.7 (s, 1H), 8.60 (s, 1H), 8.26 (d, J=8.0 Hz, 1H), 8.00 (d, J=8.8 Hz, 1H), 7.90 (d, J=8.0 Hz, 1H), 7.81 (d, J=8.8 Hz, 1H), 7.63 (t, J=8.0 Hz, 1H), 7.15 (s, 1H), 6.74 (s, 1H), 5.49 (s, 2H), 3.40-3.50 (m, 2H), 2.86 (s, 3H), 2.50-2.60 (m, 1H), 2.26-2.33 (m, 1H).

### Example 5: (R)-3-(3-(3-(3-Amino-1-methyl-1H-pyrazolo[4,3-b]pyridin-5-yl)phenyl)isoxazol-5-yl)-3-hydroxy-1-methylpyrrolidin-2-one

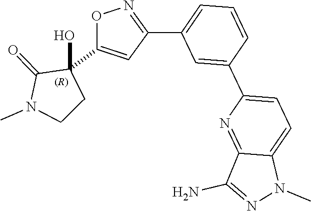

Step A: (R)-3-Hydroxy-1-methyl-3-(3-(3-(1-methyl-3-nitro-1H-pyrazolo[4,3-b]pyridin-5-yl)phenyl)isoxazol-5-yl)pyrrolidin-2-one. To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 271 mg, 0.710 mmol) in DMF (2 mL) and H₂O (0.2 mL) was added 5-chloro-1-methyl-3-nitro-1H-pyrazolo[4,3-b]pyridine (26 mg, 0.13 mmol), followed by Pd(t-Bu₃P)₂ (36 mg, 0.071 mmol) and K₃PO₄ (449 mg, 2.12 mmol). The mixture was heated at 90° C. for 16 h under a N₂ atmosphere. The mixture was cooled to rt, filtered and purified by reverse phase HPLC using an HPLC column, such as an Xtimate C18 10 μm, 21.2×250 mm column using an 18% to 35% gradient of CH₃CN/H₂O (10 mM NH₄HCO₃ and 0.025% NH₄OH) to yield (R)-3-hydroxy-1-methyl-3-(3-(3-(1-methyl-3-nitro-1H-pyrazolo[4,3-b]pyridin-5-yl)phenyl)isoxazol-5-yl)pyrrolidin-2-one (37 mg, 12%). MS (ESI): Mass calcd. for C₂₁H₁₈N₆O₅, 434.4; m/z found, 435.0 [M+H]⁺.

Step B: (R)-3-(3-(3-(3-Amino-1-methyl-1H-pyrazolo[4,3-b]pyridin-5-yl)phenyl)isoxazol-5-yl)-3-hydroxy-1-methylpyrrolidin-2-one. A solution of (R)-3-hydroxy-1-methyl-3-(3-(3-(1-methyl-3-nitro-1H-pyrazolo[4,3-b]pyridin-5-yl)phenyl)isoxazol-5-yl)pyrrolidin-2-one (30 mg, 0.07 mmol), saturated aqueous NH₄Cl solution (10 mL), and zinc dust (45 mg, 0.69 mmol) in MeOH (10 mL) was heated at 37° C. for 16 h. The reaction mixture was concentrated to dryness and purified by reverse phase HPLC using an HPLC column, such as an Xtimate C18 10 μm, 21.2×250 mm column using a 5% gradient of CH₃CN/H₂O (10 mM NH₄HCO₃ and 0.025% NH₄OH) for 3 min then a 27-35% gradient of CH₃CN/H₂O (10 mM NH₄HCO₃ and 0.025% NH₄OH) to afford (R)-3-(3-(3-(3-amino-1-methyl-1H-pyrazolo[4,3-b]pyridin-5-yl)phenyl)isoxazol-5-yl)-3-hydroxy-1-methylpyrrolidin-2-one (8.6 mg, 31%) as a yellow solid. MS (ESI): Mass calcd. for C₂₁H₂₀N₆O₃, 404.2; m/z found, 405.0 [M+H]⁺. ¹H NMR (400 MHz, DMSO-d₆) δ 9.06 (s, 1H), 8.85 (d, J=5.2 Hz, 1H), 8.68 (s, 1H), 8.13 (d, J=2.8 Hz, 1H), 7.98 (dd, J=1.2, 5.2 Hz, 1H), 7.84 (s, 1H), 7.65 (d, J=2.4 Hz, 1H), 7.48 (s, 1H), 6.83 (s, 1H), 4.00 (s, 3H), 3.43-3.52 (m, 2H), 2.86 (s, 3H), 2.56-2.62 (m, 1H), 2.29-2.34 (m, 1H).

### Example 6: (R)-3-(5-(3-(3-Amino-1-methyl-1H-pyrazolo[4,3-b]pyridin-5-yl)phenyl)isoxazol-3-yl)-3-hydroxy-1-methylpyrrolidin-2-one

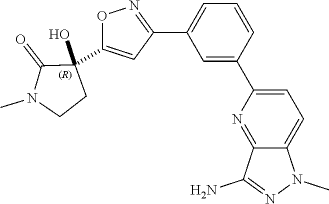

(R)-3-(5-(3-(3-Amino-1-methyl-1H-pyrazolo[4,3-b]pyridin-5-yl)phenyl)isoxazol-3-yl)-3-hydroxy-1-methylpyrrolidin-2-one was prepared using analogous conditions as described in Example 5 using (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₁H₂₀N₆O₃, 404.2; m/z found, 405.0 [M+H]⁺. ¹H NMR (400 MHz, DMSO-d₆) δ 12.30 (s, 1H), 8.70 (s, 1H), 8.61 (s, 1H), 8.55 (d, J=2.8 Hz, 1H), 8.42 (d, J=8.0 Hz, 1H), 8.34 (d, J=2.8 Hz, 1H), 7.73 (d, J=8.0 Hz, 1H), 7.57 (t, J=8.0 Hz, 1H), 7.07 (s, 1H), 6.75 (s, 1H), 3.52-3.41 (m, 2H), 2.85 (s, 3H), 2.62-2.55 (m, 1H), 2.32-2.25 (m, 1H).

### Example 7: (R)-3-Amino-6-(3-(5-(3-hydroxy-1-methyl-2-oxopyrrolidin-3-yl)isoxazol-3-yl)phenyl)picolinamide

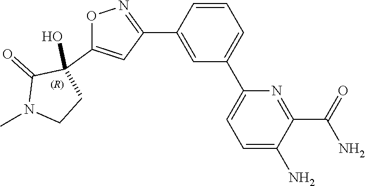

To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 105 mg, 0.270 mmol) in DMF (2 mL) and H₂O (0.2 mL) was added 3-amino-6-bromopicolinamide (70 mg, 0.4 mmol), followed by Pd(t-Bu₃P)₂ (14 mg, 0.026 mmol) and K₃PO₄ (174 mg, 0.820 mmol). The mixture was heated at 90° C. for 16 h under a N₂ atmosphere. The mixture was then cooled to rt and concentrated. The residue was purified by reverse phase HPLC using an HPLC column, such as an Xtimate 10 μm, 150 Å, 21.2×250 mm column using a 20 to 35% gradient of CH₃CN/H₂O (10 mM NH₄HCO₃ and 0.025% NH₄OH) to afford (R)-3-amino-6-(3-(5-(3-hydroxy-1-methyl-2-oxopyrrolidin-3-yl)isoxazol-3-yl)phenyl)picolinamide as a brown solid (5.2 mg, 5%). MS (ESI): Mass calcd. for C₂₀H₁₉N₅O₄, 393.1; m/z found, 394.0 [M+H]+, ¹H NMR (400 MHz, DMSO-d₆) δ 8.44 (s, 1H), 8.26 (d, J=8.0 Hz, 1H), 8.20 (s, 1H), 8.00 (d, J=8.8 Hz, 1H), 7.83 (d, J=8.0 Hz, 1H), 7.55 (t, J=8.0 Hz, 1H), 7.47 (s, 1H), 7.26 (d, J=8.8 Hz, 1H), 7.22 (s, 2H), 7.04 (s, 1H), 6.73 (s, 1H), 3.51-3.44 (m, 2H), 2.85 (s, 3H), 2.55-2.48 (m, 1H), 2.35-2.25 (m, 1H).

### Example 8: (R)-6-(3-(5-(3-Hydroxy-1-methyl-2-oxopyrrolidin-3-yl)isoxazol-3-yl)phenyl)-4-methoxypicolinamide

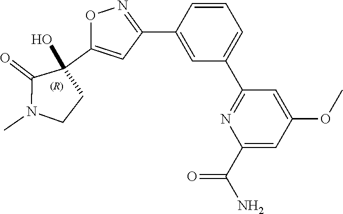

(R)-6-(3-(5-(3-Hydroxy-1-methyl-2-oxopyrrolidin-3-yl)isoxazol-3-yl)phenyl)-4-methoxypicolinamide (2 mg, 2%) was prepared using analogous conditions to those described in Example 7 using 6-chloro-4-methoxypicolinamide (Intermediate 15) in place of 3-amino-6-bromopicolinamide. MS (ESI): Mass calcd. for C₂₁H₂₀N₄O₅, 408.1; m/z found, 409.0 [M+H]⁺. ¹H NMR (400 MHz, DMSO-d₆) δ 8.63 (s, 1H), 8.46 (d, J=8.0 Hz, 1H), 8.41 (s, 1H), 7.99 (d, J=8.0 Hz, 1H), 7.83 (s, 1H), 7.78 (s, 1H), 7.64 (t, J=8.0 Hz, 1H), 7.56 (s, 1H), 7.30 (s, 1H), 6.77 (s, 1H), 4.00 (s, 3H), 3.60-3.40 (m, 2H), 2.85 (s, 3H), 2.62-2.52 (m, 1H), 2.36-2.23 (m, 1H).

### Example 9: (R,S)-3-(5-(3-(1H-Pyrrolo[2,3-b]pyridin-3-yl)phenyl)isoxazol-3-yl)-3-hydroxy-1-methylpyrrolidin-2-one

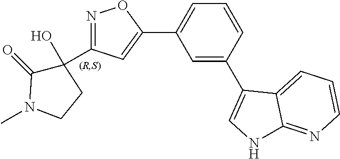 [SMILES: CN1CCC(O)(c2cc(-c3cccc(-c4c[nH]c5ncccc45)c3)on2)C1=O]

Step A: tert-Butyl 3-(3-(3-(3-hydroxy-1-methyl-2-oxopyrrolidin-3-yl)isoxazol-5-yl)phenyl)-1H-pyrrolo[2,3-b]pyridine-1-carboxylate. tert-Butyl 3-(3-(3-(3-hydroxy-1-methyl-2-oxopyrrolidin-3-yl)isoxazol-5-yl)phenyl)-1H-pyrrolo[2,3-b]pyridine-1-carboxylate (80 mg, 76%) was prepared using analogous conditions to those described in Example 7 using (R,S)-3-(5-(3-bromophenyl)isoxazol-3-yl)-3-hydroxy-1-methylpyrrolidin-2-one (Intermediate 6) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and tert-butyl 3-(4,4,5,5-tetramethyl-1,3,2-dioxaborolan-2-yl)-1H-pyrrolo[2,3-b]pyridine-1-carboxylate (Intermediate 24) in place of 3-amino-6-bromopicolinamide. MS (ESI): Mass calcd. for C₂₆H₂₆N₄O₅, 474.2; m/z found, 475.2 [M+H]⁺.

Step: B: (R,S)-3-(5-(3-(1H-Pyrrolo[2,3-b]pyridin-3-yl)phenyl)isoxazol-3-yl)-3-hydroxy-1-methylpyrrolidin-2-one. A solution of tert-butyl 3-(3-(3-(3-hydroxy-1-methyl-2-oxopyrrolidin-3-yl)isoxazol-5-yl)phenyl)-1H-pyrrolo[2,3-b]pyridine-1-carboxylate (80 mg, 0.17 mmol) in 4 M HCl in 1,4-dioxane (15 mL, 60 mmol) was stirred at rt for 16 h and then concentrated to dryness. The residue was diluted with saturated aqueous NaHCO₃ solution and extracted with EtOAc (2×15 mL). The organic solvent extracts were combined, concentrated to dryness and purified by reverse phase HPLC using an HPLC column, such as an Agela Durashell C18 10 μm, 21.2×250 mm column using a 18 to 35% gradient of CH₃CN/H₂O (10 mM NH₄HCO₃ and 0.025% NH₄OH) to afford (R,S)-3-(5-(3-(1H-pyrrolo[2,3-b]pyridin-3-yl)phenyl)isoxazol-3-yl)-3-hydroxy-1-methylpyrrolidin-2-one (42 mg, 67%) as a white solid. MS (ESI): Mass calcd. for C₂₁H₁₈N₄O₃, 374.1; m/z found, 375.2 [M+H]+, ¹H NMR (400 MHz, DMSO-d₆) δ 12.05 (s, 1H), 8.35 (d, J=8.0 Hz, 1H), 8.30 (d, J=4.0 Hz, 1H), 8.18 (s, 1H), 8.08 (s, 1H), 7.88 (t, J=8.0 Hz, 1H), 7.74 (d, J=8.0 Hz, 1H), 7.60 (t, J=8.0 Hz, 1H), 7.21 (s, 1H), 7.19 (t, J=4.0 Hz, 1H), 6.50 (s, 1H), 3.45-3.38 (m, 2H), 2.81 (s, 3H), 2.70-2.64 (m, 1H), 2.28-2.23 (m, 1H).

### Example 10: (R)-3-(3-(3-(8-Aminopyrido[3,4-d]pyrimidin-2-yl)phenyl)isoxazol-5-yl)-3-hydroxy-1-methylpyrrolidin-2-one

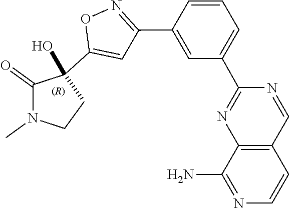

Step A: (R)-3-(3-(3-(8-Chloropyrido[3,4-d]pyrimidin-2-yl)phenyl)isoxazol-5-yl)-3-hydroxy-1-methylpyrrolidin-2-one. To a suspension of 8-chloro-2-(methylthio)pyrido[3,4-d]pyrimidine (Intermediate 13, 90.0 mg, 0.43 mmol), (R)-(3-(5-(3-hydroxy-1-methyl-2-oxopyrrolidin-3-yl)isoxazol-3-yl)phenyl)boronic acid (Intermediate 10, 154 mg, 0.51 mmol) and copper(I) thiophene-2-carboxylate (162 mg, 0.85 mmol) in THF (2 mL) was added Pd(PPh₃)₄ (49 mg, 0.04 mmol). The mixture was heated at 105° C. under a nitrogen atmosphere for 2 h. The mixture was cooled to rt and concentrated under reduced pressure. The residue was purified by preparative TLC (DCM/MeOH=20/1, R_{f}=0.4) to afford (R)-3-(3-(3-(8-chloropyrido[3,4-d]pyrimidin-2-yl)phenyl)isoxazol-5-yl)-3-hydroxy-1-methylpyrrolidin-2-one (40 mg, 22%) as a yellow solid. MS (ESI): Mass calcd. for C₂₁H₁₆ClN₅O₃, 421.1; m/z found, 422.1 [M+H]⁺.

Step B: (R)-3-(3-(3-(8-Aminopyrido[3,4-d]pyrimidin-2-yl)phenyl)isoxazol-5-yl)-3-hydroxy-1-methylpyrrolidin-2-one. To a solution of (R)-3-(3-(3-(8-chloropyrido[3,4-d]pyrimidin-2-yl)phenyl)isoxazol-5-yl)-3-hydroxy-1-methylpyrrolidin-2-one (50 mg, 0.1 mmol) in dioxane (1 mL) was added aqueous ammonium hydroxide (12 N, 5 mL, 60 mmol). The mixture was heated at 70° C. for 16 h in a sealed tube and then concentrated under reduced pressure. The residue was purified by reverse phase HPLC using an HPLC column, such as a Boston pHlex ODS 10 μm, 21.2×250 mm column using a 30 to 54% gradient of CH₃CN and H₂O (0.1% FA) to afford (R)-3-(3-(3-(8-aminopyrido[3,4-d]pyrimidin-2-yl)phenyl)isoxazol-5-yl)-3-hydroxy-1-methylpyrrolidin-2-one (1 mg, 2%) as a yellow solid. MS (ESI): Mass calcd. for C₂₁H₁₈N₆O₃, 402.1; m/z found, 403.1 [M+H]⁺. ¹H NMR (400 MHz, CD₃OD) δ 9.48 (s, 1H), 9.14-9.16 (m, 1H), 8.78 (d, J=8.0 Hz, 1H), 8.03 (d, J=8.0 Hz, 1H), 7.96 (d, J=5.6 Hz, 1H), 7.70 (d, J=8.0 Hz, 1H), 7.10 (s, 1H), 7.09 (d, J=5.6 Hz, 1H), 3.63-3.60 (m, 2H), 3.00 (s, 3H), 2.80-2.76 (m, 1H), 2.46-2.42 (m, 1H).

### Example 11: (R)-3-(3-(3-(4-(Azetidin-1-yl)pyrido[3,2-d]pyrimidin-6-yl)phenyl)isoxazol-5-yl)-3-hydroxy-1-methylpyrrolidin-2-one

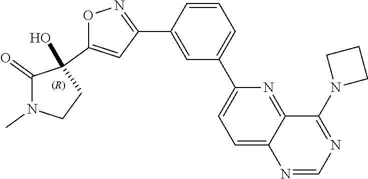

To a solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 140 mg, 0.36 mmol) in DMF (3 mL) and H₂O (0.3 mL) was added 4-(azetidin-1-yl)-6-chloropyrido[3,2-d]pyrimidine (80 mg, 0.4 mmol), followed by Pd(t-Bu₃P)₂ (18 mg, 0.04 mmol) and K₂CO₃ (151 mg, 1.09 mmol). The mixture was heated at 105° C. for 16 h under a N₂ atmosphere and then cooled to rt and concentrated. The resulting compound was purified using reverse phase HPLC using an HPLC column, such as an Xtimate C18 10 μm, 21.2×250 mm column using a 36 to 41% gradient of CH₃CN/H₂O (10 mM NH₄HCO₃ and 0.025% NH₄OH) to afford (R)-3-(3-(3-(4-(azetidin-1-yl)pyrido[3,2-d]pyrimidin-6-yl)phenyl)isoxazol-5-yl)-3-hydroxy-1-methylpyrrolidin-2-one (37 mg, 23%) as an off-white solid. MS (ESI): Mass calcd. for C₂₄H₂₂N₆O₃, 442.47; m/z found, 443.2 [M+H]⁺. ¹H NMR (400 MHz, DMSO-d₆) δ 8.68 (s, 1H), 8.56 (d, J=8.8 Hz, 1H), 8.45 (s, 1H), 8.34 (d, J=7.6, 1H), 8.17 (d, J=8.8 Hz, 1H), 8.01 (d, J=7.6, 1H), 7.17 (t, J=7.6, 1H), 7.19 (s, 1H), 6.77 (s, 1H), 5.04 (t, J=7.2, 2H), 4.34 (t, J=7.2, 2H), 3.50-3.44 (m, 2H), 2.86 (s, 3H), 2.62-2.57 (m, 2H), 2.33-2.27 (m, 2H).

### Example 12: (R)-3-(3-(3-(7-Aminothiazolo[5,4-d]pyrimidin-2-yl)-4-methylphenyl)isoxazol-5-yl)-3-hydroxy-1-methylpyrrolidin-2-one

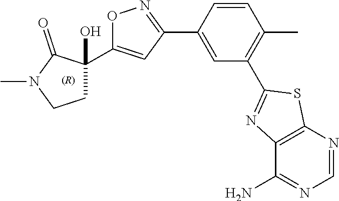

Step A: 3-(7-Aminothiazolo[5,4-d]pyrimidin-2-yl)-4-methylbenzaldehyde. A mixture of 2-(5-iodo-2-methylphenyl)thiazolo[5,4-d]pyrimidin-7-amine (Intermediate 20, 400 mg, 1 mmol), 1,1′-bis(diphenylphosphino)ferrocene-palladium(II)dichloride DCM complex (44 mg, 0.054 mmol), triethylsilane (379 mg, 3.26 mmol) and Na₂CO₃ (138 mg, 1.30 mmol) in DMF (8 mL) were heated at 80° C. for 24 h under a CO atmosphere. The reaction mixture was cooled, diluted with EtOAc (20 mL) and washed with brine (3×10 mL). The organic solvent portion was separated, dried over Na₂SO₄, filtered and concentrated. The residue was purified by preparative TLC (DCM/MeOH=20/1) to afford 3-(7-aminothiazolo[5,4-d]pyrimidin-2-yl)-4-methylbenzaldehyde (0.22 g, 75%) as a yellow solid. MS (ESI): Mass calcd. for C₁₃H₁₀N₄OS, 270.3; m/z found, 271.1 [M+H]⁺.

Step B: 3-(7-Aminothiazolo[5,4-d]pyrimidin-2-yl)-4-methylbenzaldehyde oxime. To a solution of 3-(7-aminothiazolo[5,4-d]pyrimidin-2-yl)-4-methylbenzaldehyde (220 mg, 0.81 mmol) in EtOH (10 mL) was added hydroxylamine (50% in water) (0.24 mL, 4.07 mmol). The mixture was stirred at 25° C. for 2 h and then concentrated to afford 3-(7-aminothiazolo[5,4-d]pyrimidin-2-yl)-4-methylbenzaldehyde oxime (200 mg, 86%) as a white solid. MS (ESI): Mass calcd. for C₁₃H₁₁N₅OS, 285.3; m/z found, 286.1 [M+H]⁺.

Step C: 3-(7-Aminothiazolo[5,4-d]pyrimidin-2-yl)-N-hydroxy-4-methylbenzimidoyl chloride. To a solution of 3-(7-aminothiazolo[5,4-d]pyrimidin-2-yl)-4-methylbenzaldehyde oxime (200 mg, 0.7 mmol) in DCM (10 mL) and DMF (15 mL) was added N-chlorosuccinimide (281 mg, 2.10 mmol) portion-wise. The reaction mixture was stirred at 25° C. for 5 h, then diluted with EtOAc (50 mL) and washed with brine (3×60 mL). The organic solvent portion was dried over Na₂SO₄, filtered and concentrated to afford 3-(7-aminothiazolo[5,4-d]pyrimidin-2-yl)-N-hydroxy-4-methylbenzimidoyl chloride (0.18 g, 80%) as a yellow solid. MS (ESI): Mass calcd. for C₁₃H₁₀ClN₅OS, 319.8; m/z found, 284.1 [M−HCl]⁺.

Step D: (R)-3-(3-(3-(7-Aminothiazolo[5,4-d]pyrimidin-2-yl)-4-methylphenyl)isoxazol-5-yl)-3-hydroxy-1-methylpyrrolidin-2-one. To a solution of 3-(7-aminothiazolo[5,4-d]pyrimidin-2-yl)-N-hydroxy-4-methylbenzimidoyl chloride (180 mg, 0.28 mmol) in DCM (15 mL) was added (R)-3-ethynyl-3-hydroxy-1-methylpyrrolidin-2-one (Intermediate 1, 39.2 mg, 0.28 mmol), followed by triethylamine (0.12 mL, 0.84 mmol). The reaction mixture was stirred at 25° C. for 16 h, diluted with DCM (30 mL) and washed with brine (20 mL). The organic solvent portion was dried over Na₂SO₄, filtered and concentrated. The residue was purified by reverse phase HPLC using an HPLC column, such as a Boston pHlex ODS 10 μm, 21.2×250 mm column using a 20% to 60% gradient of CH₃CN/H₂O (0.1% FA) to afford (R)-3-(3-(3-(7-aminothiazolo[5,4-d]pyrimidin-2-yl)-4-methylphenyl)isoxazol-5-yl)-3-hydroxy-1-methylpyrrolidin-2-one (1.9 mg, 1%) as a yellow oil. MS (ESI): Mass calcd. for C₂₀H₁₈N₆O₃S, 422.1; m/z found, 423.1 [M+H]⁺. ¹H NMR (400 MHz, DMSO-d₆) δ 8.43 (s, 4H), 8.29 (s, 1H), 8.20 (d, J=1.2 Hz, 1H), 7.87 (dd, J=8.0, 1.6 Hz, 1H), 8.51 (d, J=8.0 Hz, 1H), 6.96 (s, 1H), 3.41-3.47 (m, 2H), 2.84 (s, 3H), 2.66 (s, 3H), 2.54-2.61 (m, 1H), 2.26-2.32 (m, 1H).

### Example 13: (R)-3-(3-(3-(7-Aminothiazolo[5,4-d]pyrimidin-2-yl)phenyl)isoxazol-5-yl)-3-hydroxy-1-methylpyrrolidin-2-one

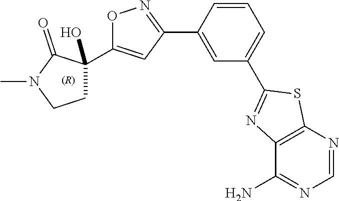

(R)-3-(3-(3-(7-Aminothiazolo[5,4-d]pyrimidin-2-yl)phenyl)isoxazol-5-yl)-3-hydroxy-1-methylpyrrolidin-2-one was prepared using analogous conditions to those described in Example 1 using 2-(3-iodophenyl)thiazolo[5,4-d]pyrimidin-7-amine (Intermediate 21) in place of 6-chloro-2-methylpyrido[3,2-d]pyrimidin-4-amine (Intermediate 16). MS (ESI): Mass calcd. for C₁₉H₁₆N₆O₃S, 408.1; m/z found, 409 [M+H]⁺. ¹H NMR (400 MHz, DMSO-d₆) δ 8.56 (s, 1H), 8.33 (s, 1H), 8.17 (d, J=8.0 Hz, 1H), 8.08 (d, J=8.0 Hz, 1H), 7.85 (s, 2H), 7.74 (t, J=8.0 Hz, 1H), 7.15 (s, 1H), 6.78 (s, 1H), 3.53-3.44 (m, 2H), 2.85 (s, 3H), 2.62-2.55 (m, 1H), 2.33-2.26 (m, 1H).

### Example 14: (R)-6-(3-(4-(3-Hydroxy-1-methyl-2-oxopyrrolidin-3-yl)-1H-1,2,3-triazol-1-yl)phenyl)picolinamide

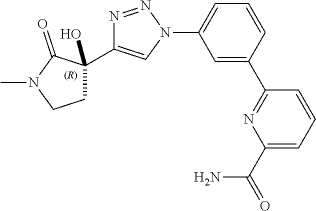

Step A: Methyl (R)-6-(3-(4-(3-hydroxy-1-methyl-2-oxopyrrolidin-3-yl)-1H-1,2,3-triazol-1-yl)phenyl)picolinate. To a mixture of methyl 6-(3-iodophenyl)picolinate (64 mg, 0.19 mmol), (R)-3-ethynyl-3-hydroxy-1-methylpyrrolidin-2-one (Intermediate 1, 32 mg, 0.23 mmol), NaN₃ (13.5 mg, 0.210 mmol), CuI (7 mg, 0.04 mmol) and trans-N,N′-dimethyl-cyclohexane-1,2-diamine (5.4 mg, 0.038 mmol) in DMSO (2 mL) was added water (0.5 mL) followed by sodium ascorbate (7.5 mg, 0.038 mmol). The mixture was stirred at rt under a N₂ atmosphere for 16 h, poured into water (20 mL) and extracted with EtOAc (2×20 mL). The combined organic extracts were washed with brine (2×20 mL), dried over Na₂SO₄, filtered, and concentrated to dryness. The residue was purified by preparative TLC (EA/petroleum ether=2/1, R_{f}=0.5) to afford methyl (R)-6-(3-(4-(3-hydroxy-1-methyl-2-oxopyrrolidin-3-yl)-1H-1,2,3-triazol-1-yl)phenyl)picolinate (70 mg, 94%) as a white solid. MS (ESI): Mass calcd. for C₂₀H₁₉N₅O₄, 393.4; m/z found, 394 [M+H]⁺.

Step B: (R)-6-(3-(4-(3-Hydroxy-1-methyl-2-oxopyrrolidin-3-yl)-1H-1,2,3-triazol-1-yl)phenyl)picolinamide. A mixture of methyl (R)-6-(3-(4-(3-hydroxy-1-methyl-2-oxopyrrolidin-3-yl)-1H-1,2,3-triazol-1-yl)phenyl)picolinate (60 mg, 0.15 mmol) and ammonia in MeOH (7 N, 5 mL) was heated at 75° C. for 16 h. The solution was concentrated and purified by reverse phase HPLC using an HPLC column, such as an Xtimate 10 μm 150 Å, 21.2×250 mm column using a 5% to 33% gradient of CH₃CN/H₂O (10 mM NH₄HCO₃ and 0.025% NH₄OH) to afford (R)-6-(3-(4-(3-hydroxy-1-methyl-2-oxopyrrolidin-3-yl)-1H-1,2,3-triazol-1-yl)phenyl)picolinamide (19 mg, 33%) as a white solid. MS (ESI): Mass calcd. for C₁₉H₁₈N₆O₃, 378.4; m/z found, 379.1 [M+H]⁺. ¹H NMR (400 MHz, DMSO-d₆) δ 8.96 (s, 1H), 8.73 (s, 1H), 8.52 (s, 1H), 8.43 (d, J=7.6 Hz, 1H), 8.37 (d, J=8.0, 1H), 8.12 (t, J=7.6 Hz, 1H), 8.05 (d, J=7.6 Hz, 2H), 7.80-7.70 (m, 2H), 6.28 (s, 1H), 3.55-3.45 (m, 2H), 2.83 (s, 3H), 2.80-2.65 (m, 1H), 2.35-2.25 (m, 1H).

### Example 15: (R)-1-(3-(4-(3-Hydroxy-1-methyl-2-oxopyrrolidin-3-yl)-1H-1,2,3-triazol-1-yl)phenyl)imidazo[1,5-a]pyridine-3-carboxamide

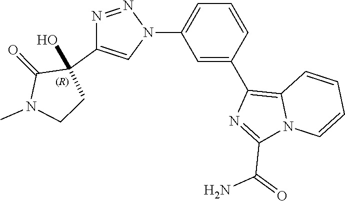

To a solution of (R)-3-hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (Intermediate 8, 100 mg, 0.26) in DMF (10 mL) and water (1 mL) was added 1-bromoimidazo[1,5-a]pyridine-3-carboxamide (62 mg, 0.26 mmol), followed by Pd(t-Bu₃P)₂ (13 mg, 0.026 mmol) and K₃PO₄ (166 mg, 0.78 mmol). The mixture was heated at 90° C. for 16 h under a nitrogen atmosphere, cooled to rt, and then concentrated under reduced pressure. The residue was purified by preparative HPLC to afford (R)-1-(3-(4-(3-hydroxy-1-methyl-2-oxopyrrolidin-3-yl)-1H-1,2,3-triazol-1-yl)phenyl)imidazo[1,5-a]pyridine-3-carboxamide (19.4 mg, 18%) as a yellow solid. MS (ESI): Mass calcd. for C₂₁H₁₉N₇O₃, 417.2; m/z found, 418.0 [M+H]⁺. ¹H NMR (400 MHz, DMSO-d₆) δ 9.51 (d, J=7.6 Hz, 1H), 8.79 (s, 1H), 8.45 (t, J=2 Hz, 1H), 8.24 (d, J=9.2 Hz, 1H), 8.10 (d, J=8.4 Hz, 2H), 7.88-7.85 (m, 1H), 7.74-7.65 (m, 2H), 7.29-7.26 (m, 1H), 7.09 (t, J=6.8 Hz, 1H), 6.28 (s, 1H), 3.51-3.47 (m, 2H), 2.83 (s, 3H), 2.77-2.66 (m, 1H), 2.34-2.26 (m, 1H).

### Example 16: (R)-3-(5-(3-(4-Aminopyrido[3,2-d]pyrimidin-6-yl-2-d)phenyl)isoxazol-3-yl)-3-hydroxy-1-methylpyrrolidin-2-one

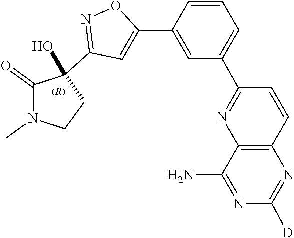

A mixture of 6-chloropyrido[3,2-d]pyrimidin-2-d-4-amine (Intermediate 17, 78 mg, 0.43 mmol), (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5, 110 mg, 0.29 mmol), bis(tri-tert-butylphosphine)palladium(0) (15 mg, 0.029 mmol), K₃PO₄ (182 mg, 0.86 mmol), 1,4-dioxane (10 mL) and water (1 mL) was heated at 65° C. for 16 h under an argon atmosphere. The reaction mixture was filtered and concentrated. The residue was purified by reverse phase HPLC using an HPLC column, such as an Xtimate C18 10 μm, 21.2×250 mm column using a 20% to 35% gradient of CH₃CN/H₂O (10 mM NH₄HCO₃ and 0.025% NH₄OH) to afford (R)-3-(5-(3-(4-aminopyrido[3,2-d]pyrimidin-6-yl-2-d)phenyl)isoxazol-3-yl)-3-hydroxy-1-methylpyrrolidin-2-one (51 mg, 44%) as a white solid. MS (ESI): Mass calcd. for C₂₁H₁₇DN₆O₃, 403.4; m/z found, 404.2 [M+H]⁺. ¹H NMR (400 MHz, DMSO-d₆) δ 8.86 (s, 1H), 8.61 (d, J=4.8 Hz, 1H), 8.57 (d, J=8 Hz, 1H), 8.31 (s, 1H), 8.17 (d, J=4.8 Hz, 1H), 8.05 (s, 1H), 7.98 (d, J=8 Hz, 1H), 7.71 (t, J=8 Hz, 1H), 7.40 (s, 1H), 6.53 (s, 1H), 3.49-3.38 (m, 2H), 2.82 (s, 3H), 2.72-2.65 (m, 1H), 2.31-2.23 (m, 1H).

### Example 17: (R)-3-(5-(3-(3-Amino-1H-pyrazolo[4,3-b]pyridin-5-yl)phenyl)isoxazol-3-yl)-3-hydroxy-1-methylpyrrolidin-2-one

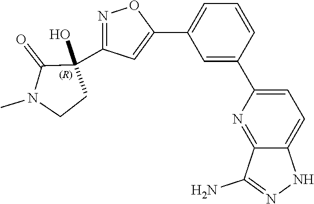

Step A: (R)-3-(5-(3-(3-Nitro-1H-pyrazolo[4,3-b]pyridin-5-yl)phenyl)isoxazol-3-yl)-3-hydroxy-1-methylpyrrolidin-2-one. To a solution of (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5, 100 mg, 0.26 mmol) in 1,4-dioxane (4 mL), H₂O (1 mL) and DMF (5 mL) were added 5-chloro-3-nitro-1H-pyrazolo[4,3-b]pyridine (103 mg, 0.52 mmol), Pd(t-Bu₃P)₂ (13 mg, 0.026 mmol) and K₃PO₄ (166 mg, 0.78 mmol). The mixture was heated at 80° C. for 16 h under an Ar atmosphere, cooled to rt, and then concentrated. The residue was purified by reverse phase HPLC using an HPLC column, such as an Xtimate C18 10 μm, 21.2×250 mm column using a 15% to 35% gradient of CH₃CN/H₂O (10 mM NH₄HCO₃ and 0.025% NH₄OH) to afford (R)-3-(5-(3-(3-nitro-1H-pyrazolo[4,3-b]pyridin-5-yl)phenyl)isoxazol-3-yl)-3-hydroxy-1-methylpyrrolidin-2-one (20 mg, 18%) as a white solid. MS (ESI): Mass calcd. for C₂₀H₁₆N₆O₅, 420.4; m/z found, 421.2 [M+H]⁺.

Step B: (R)-3-(5-(3-(3-Amino-1H-pyrazolo[4,3-b]pyridin-5-yl)phenyl)isoxazol-3-yl)-3-hydroxy-1-methylpyrrolidin-2-one. To a solution of (R)-3-(5-(3-(3-nitro-1H-pyrazolo[4,3-b]pyridin-5-yl)phenyl)isoxazol-3-yl)-3-hydroxy-1-methylpyrrolidin-2-one (20 mg, 0.05 mmol) in saturated aqueous NH₄Cl solution (2 mL) and MeOH (2 mL) was added zinc dust (200 mg, 3.06 mmol). The mixture was stirred at 30° C. for 1 h, filtered and the filtrate was concentrated. The residue was purified by reverse phase HPLC using an HPLC column, such as an Xtimate C18 10 μm, 21.2×250 mm column using a 5% to 40% gradient of CH₃CN/H₂O (10 mM NH₄HCO₃ and 0.025% NH₄OH) to afford (R)-3-(5-(3-(3-amino-1H-pyrazolo[4,3-b]pyridin-5-yl)phenyl)isoxazol-3-yl)-3-hydroxy-1-methylpyrrolidin-2-one (1 mg, 5%) as a pale yellow solid. MS (ESI): Mass calcd. for C₂₀H₁₈N₆O₃, 390.4; m/z found, 391.2 [M+H]⁺. ¹H NMR (400 MHz, CD₃OD) δ 8.54 (s, 1H), 8.15 (d, J=8.0 Hz, 1H), 7.95-7.88 (m, 3H), 7.66 (t, J=8.0 Hz, 1H), 7.07 (s, 1H), 3.58-3.55 (m, 2H), 2.95 (s, 3H), 2.88-2.80 (m, 1H), 2.41-2.37 (m, 1H).

### Example 18: (R)-6-(3-(3-(3-Hydroxy-1-methyl-2-oxopyrrolidin-3-yl)isoxazol-5-yl)phenyl)picolinamide

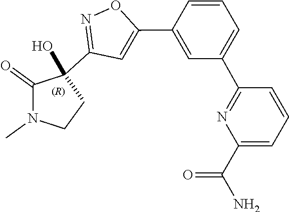

Step A: Methyl (R)-6-(3-(3-(3-hydroxy-1-methyl-2-oxopyrrolidin-3-yl)isoxazol-5-yl)phenyl)picolinate. A mixture of methyl 6-bromopicolinate (76 mg, 0.35 mmol), (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5, 90 mg, 0.23 mmol), bis(tri-tert-butylphosphine)palladium(0) (11.9 mg, 0.02 mmol) and K₃PO₄ (149 mg, 0.70 mmol) in 1,4-dioxane (8 mL) and water (1 mL) was heated at 75° C. for 16 h under an Ar atmosphere. The reaction mixture was filtered, concentrated and the residue was purified by preparative TLC (DCM:MeOH=20:1) to afford methyl (R)-6-(3-(3-(3-hydroxy-1-methyl-2-oxopyrrolidin-3-yl)isoxazol-5-yl)phenyl)picolinate (87 mg, 94%) as a white solid. MS (ESI): Mass calcd. for C₂₁H₁₉N₃O₅, 393.1; m/z found, 394.1 [M+H]⁺.

Step B: (R)-6-(3-(3-(3-Hydroxy-1-methyl-2-oxopyrrolidin-3-yl)isoxazol-5-yl)phenyl)picolinamide. A solution of methyl (R)-6-(3-(3-(3-hydroxy-1-methyl-2-oxopyrrolidin-3-yl)isoxazol-5-yl)phenyl)picolinate (75.9 mg, 0.35 mmol) in NH₃ (7 N, in EtOH, 5 mL) was heated at 75° C. for 16 h. The reaction mixture was concentrated and the residue was purified by reverse phase HPLC using an HPLC column, such as an Xtimate 10 μm, 21.2×250 mm column using a 15 to 45% gradient of CH₃CN/H₂O (10 mM NH₄HCO₃ and 0.025% NH₄OH) to afford (R)-6-(3-(3-(3-hydroxy-1-methyl-2-oxopyrrolidin-3-yl)isoxazol-5-yl)phenyl)picolinamide (18 mg, 23%) as a white solid. MS (ESI): Mass calcd. for C₂₀H₁₈N₄O₄, 378.1; m/z found, 379.1 [M+H]⁺. ¹H NMR (400 MHz, DMSO-d₆) δ 8.73 (s, 1H), 8.47 (s, 1H), 8.45 (s, 1H), 8.35 (d, J=7.6 Hz, 1H), 8.11 (t, J=7.6 Hz, 1H), 8.03 (d, J=8.0 Hz, 1H), 7.95 (d, J=7.6 Hz, 1H), 7.76 (s, 1H), 7.68 (t, J=8.0 Hz, 1H), 7.36 (s, 1H), 6.52 (s, 1H), 3.49-3.37 (m, 2H), 2.82 (s, 3H), 2.71-2.65 (m, 1H), 2.29-2.22 (m, 1H).

### Example 19: (R,S)-2-(3-(4-(3-Hydroxy-1-methyl-2-oxopyrrolidin-3-yl)-1H-imidazol-1-yl)phenyl)quinazoline-4-carboxamide

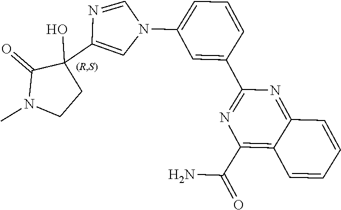

Step A: (R,S)-Ethyl 2-(3-(4-(3-hydroxy-1-methyl-2-oxopyrrolidin-3-yl)-1H-imidazol-1-yl)phenyl)quinazoline-4-carboxylate. To a solution of (R,S)-3-hydroxy-1-methyl-3-(1-(3-(4,4,5,5-tetramethyl-1,3,2-dioxaborolan-2-yl)phenyl)-1H-imidazol-4-yl)pyrrolidin-2-one (Intermediate 9, 180 mg, 0.47 mmol) in 1,4-dioxane (2 mL) and H₂O (0.2 mL) was added ethyl 2-chloroquinazoline-4-carboxylate (222 mg, 0.94 mmol) followed by Pd(t-Bu₃P)₂ (24.0 mg, 0.05 mmol) and KF (82 mg, 1.41 mmol). The mixture was heated at 85° C. overnight under a N₂ atmosphere. The mixture was concentrated and purified using preparative TLC (DCM/MeOH=15/1) to afford ethyl (R,S)-2-(3-(4-(3-hydroxy-1-methyl-2-oxopyrrolidin-3-yl)-1H-imidazol-1-yl)phenyl)quinazoline-4-carboxylate (150 mg, 69.8%) as a yellow solid. MS (ESI): Mass calcd. for C₂₅H₂₃N₅O₄, 457.5; m/z found, 458.0 [M+H]⁺.

Step B: (R,S)-2-(3-(4-(3-Hydroxy-1-methyl-2-oxopyrrolidin-3-yl)-1H-imidazol-1-yl)phenyl)quinazoline-4-carboxamide. A solution of (R,S)-ethyl 2-(3-(4-(3-hydroxy-1-methyl-2-oxopyrrolidin-3-yl)-1H-imidazol-1-yl)phenyl) quinazoline-4-carboxylate (140 mg, 0.31 mmol) in NH₃ in MeOH (7 N, 5 mL) was heated at 75° C. for 16 h. The reaction mixture was concentrated and the residue was purified by HPLC using an HPLC column, such as an Xtimate 10 μm 150 Å, 21.2×250 mm column using a 15 to 45% gradient of CH₃CN/H₂O (10 mM NH₄HCO₃ and 0.025% NH₄OH) to afford (R,S)-2-(3-(4-(3-hydroxy-1-methyl-2-oxopyrrolidin-3-yl)-1H-imidazol-1-yl)phenyl)quinazoline-4-carboxamide (40 mg, 31%) as a white solid. MS (ESI): Mass calcd. for C₂₃H₂₀N₆O₃, 428.4; m/z found, 429.0 [M+H]⁺. ¹H NMR (400 MHz, DMSO-d₆) δ 8.91 (d, J=8.4 Hz, 1H), 8.79 (s, 2H), 8.64 (d, J=8.4 Hz, 1H), 8.31 (s, 1H), 8.10-8.30 (m, 3H), 8.00-7.70 (m, 4H), 5.89 (s, 1H), 3.50-3.30 (m, 2H), 2.80 (s, 3H), 2.70-2.50 (m, 1H), 2.20-2.10 (m, 1H).

### Example 20: (R)-3-(3-(2-(8-Amino-3,4-dihydro-2,7-naphthyridin-2(1H)-yl)pyridin-4-yl)isoxazol-5-yl)-3-hydroxy-1-methylpyrrolidin-2-one

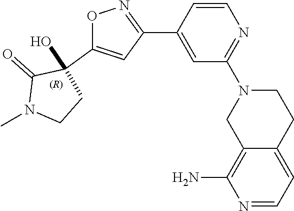

To a solution of (R)-3-(3-(2-bromopyridin-4-yl)isoxazol-5-yl)-3-hydroxy-1-methylpyrrolidin-2-one (Intermediate 7, 560 mg, 1.66 mmol) and 5,6,7,8-tetrahydro-2,7-naphthyridin-1-amine (247 mg, 1.66 mmol) in DMF (9 mL) was added Pd₂(dba)₃ (75.8 mg, 0.08 mmol), followed by BINAP (155 mg, 0.25 mmol) and t-BuONa (477 mg, 4.97 mmol). The reaction mixture was heated at 85° C. for 16 h under an Ar atmosphere, then diluted with water (20 mL) and extracted with EtOAc (3×20 mL). The combined organic solvent extracts were concentrated to dryness and purified subsequently by preparative TLC (DCM:MeOH 10:1) and reverse phase HPLC to afford (R)-3-(5-(3-(8-amino-3,4-dihydro-2,7-naphthyridin-2(1H)-yl)phenyl)isoxazol-3-yl)-3-hydroxy-1-methylpyrrolidin-2-on (2.5 mg, 0.4%) as a pale yellow solid. MS (ESI): Mass calcd. for C₂₁H₂₂N₆O₃, 406.2; m/z found, 407.2 [M+H]⁺. ¹H NMR (400 MHz, DMSO-d₆) δ 8.27 (d, J=5.2 Hz, 1H), 7.75 (d, J=5.2 Hz, 1H), 7.35 (s, 1H), 7.18 (s, 1H), 7.11 (d, J=5.2 Hz, 1H), 6.78 (s, 1H), 6.41 (d, J=5.2 Hz, 1H), 5.92 (s, 2H), 4.31 (s, 2H), 3.94 (t, J=5.2 Hz, 2H), 3.50-3.40 (m, 2H), 2.85 (s, 3H), 2.75 (t, J=5.2 Hz, 2H), 2.60-2.54 (m, 1H), 2.33-2.25 (m, 1H).

### Example 21: (R)-4′-(5-(3-Hydroxy-1-methyl-2-oxopyrrolidin-3-yl)isoxazol-3-yl)-4-methoxy-[2,2′-bipyridine]-6-carboxamide

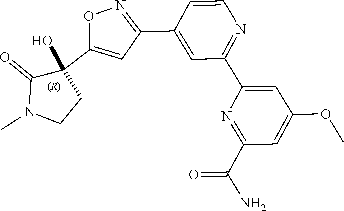

Step A: 4-Methoxy-6-(tributylstannyl)picolinamide. To a solution of 6-bromo-4-methoxypicolinamide (400.0 mg, 1.730 mmol) in toluene (10 mL), was added bis(tributyltin) (2 g, 3.5 mmol), followed by Pd(Ph₃P)₄ (100 mg, 0.09 mmol) and the reaction mixture was heated at 90° C. for 16 h under a N₂ atmosphere. A solution of saturated aqueous KF (20 mL) was added and stirring was continued at rt for 2 h. The mixture was extracted with EtOAc (3×20 mL), then the combined organic solvent extracts were washed with brine (3×20 mL) and concentrated to dryness. The residue was purified by FCC (DCM:MeOH, 20:1) to afford 4-methoxy-6-(tributylstannyl)picolinamide (185 mg, 18%) as a yellow solid. MS (ESI): Mass calcd. for C₁₉H₃₄N₂O₂Sn, 442.2; m/z found, 443.1 [M+H]⁺.

Step B: (R)-4′-(3-(3-Hydroxy-1-methyl-2-oxopyrrolidin-3-yl)isoxazol-5-yl)-4-methoxy-[2,2′-bipyridine]-6-carboxamide. A solution of 4-methoxy-6-(tributylstannyl)picolinamide (185 mg, 0.42 mmol), (R)-3-(3-(2-bromopyridin-4-yl)isoxazol-5-yl)-3-hydroxy-1-methylpyrrolidin-2-one (Intermediate 7, 212 mg, 0.06 mmol), and Pd(Ph₃P)₄ (48.5 mg, 0.04 mmol) in DMF (3 mL) was heated at 125° C. for 16 h under N₂. The reaction mixture was cooled to rt, diluted with EtOAc (20 mL) and washed with brine (3×15 mL). The organic layer was concentrated to dryness and purified by reverse phase HPLC using an HPLC column, such an Xtimate 10 μm 150 Å, 21.2×250 mm column using a 5% to 36% gradient of CH₃CN/H₂O (10 mM NH₄HCO₃ and 0.025% NH₄OH) to afford (R)-4′-(5-(3-hydroxy-1-methyl-2-oxopyrrolidin-3-yl)isoxazol-3-yl)-4-methoxy-[2,2′-bipyridine]-6-carboxamide (12 mg, 7%) as a white solid. MS (ESI): Mass calcd. for C₂₀H₁₉N₅O₅, 409.2; m/z found, 410.2 [M+H]⁺. ¹H NMR (400 MHz, DMSO-d₆) δ 8.63 (s, 1H), 8.46 (d, J=8.0 Hz, 1H), 8.41 (s, 1H), 7.99 (d, J=8.0 Hz, 1H), 7.83 (s, 1H), 7.78 (s, 1H), 7.64 (t, J=8.0 Hz, 1H), 7.56 (s, 1H), 7.30 (s, 1H), 4.00 (s, 3H), 3.60-3.40 (m, 2H), 2.85 (s, 3H), 2.62-2.52 (m, 1H), 2.36-2.23 (m, 1H).

### Example 22: (R)-1-(4-(5-(3-Hydroxy-1-methyl-2-oxopyrrolidin-3-yl)isoxazol-3-yl)pyridin-2-yl)-1H-indazole-3-carboxamide

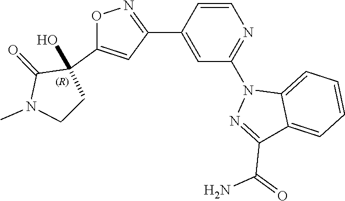

Step A: Methyl (R)-1-(4-(5-(3-hydroxy-1-methyl-2-oxopyrrolidin-3-yl)isoxazol-3-yl)pyridin-2-yl)-1H-indazole-3-carboxylate. To a solution of methyl 1H-indazole-3-carboxylate (65 mg, 0.67 mmol) in toluene (5 mL), was added (R)-3-(3-(2-bromopyridin-4-yl)isoxazol-5-yl)-3-hydroxy-1-methylpyrrolidin-2-one (Intermediate 7, 124 mg, 0.37 mmol), followed by (1 S,2S)—N¹,N²-dimethylcyclohexane-1,2-diamine (10.5 mg, 0.074 mmol), CuI (3.5 mg, 0.02 mmol), and K₃PO₄ (165 mg, 0.78 mmol). The reaction mixture was heated at 100° C. for 16 h under a N₂ atmosphere, then concentrated to dryness and purified by FCC (DCM:MeOH, 20:1) to afford methyl (R)-1-(4-(5-(3-hydroxy-1-methyl-2-oxopyrrolidin-3-yl)isoxazol-3-yl)pyridin-2-yl)-1H-indazole-3-carboxylate (60 mg, 77%) as a yellow solid. MS (ESI): Mass calcd. for C₂₂H₁₉N₅O₅, 433.1; m/z found, 434.1 [M+H]⁺.

Step B: (R)-1-(4-(3-(3-Hydroxy-1-methyl-2-oxopyrrolidin-3-yl)isoxazol-5-yl)pyridin-2-yl)-1H-indazole-3-carboxamide. A solution of methyl (R)-1-(4-(5-(3-hydroxy-1-methyl-2-oxopyrrolidin-3-yl)isoxazol-3-yl)pyridin-2-yl)-1H-indazole-3-carboxylate (60 mg, 0.14 mmol) in NH₃ (7 N, in MeOH, 5 mL, 35 mmol) was heated at 75° C. for 16 h. The mixture was cooled to rt, concentrated to dryness and purified by reverse phase HPLC using an HPLC column, such as Xtimate 10 μm 150 Å, 21.2×250 mm column using a 40 to 45% gradient of CH₃CN/(10 mM NH₄HCO₃ and 0.025% NH₄OH in H₂O) to afford (R)-1-(4-(5-(3-ydroxy-1-methyl-2-oxopyrrolidin-3-yl)isoxazol-3-yl)pyridin-2-yl)-1H-indazole-3-carboxamide (33 mg, 55%) as a white solid. MS (ESI): Mass calcd. for C₂₁H₁₈N₆O₄, 418.1; m/z found, 419.0 [M+H]⁺. ¹H NMR (400 MHz, DMSO-d₆) δ 8.84 (d, J=8.8 Hz, 1H), 8.76 (d, J=5.2 Hz, 1H), 8.69 (s, 1H), 8.35-8.32 (m, 2H), 7.88-7.86 (m, 1H), 7.74 (s, 1H), 7.65 (t, J=7.2 Hz, 1H), 7.46 (t, J=7.2 Hz, 1H), 7.30 (s, 1H), 6.85 (s, 1H), 3.52-3.45 (m, 2H), 2.86 (s, 3H), 2.62-2.57 (m, 1H), 2.35-2.30 (m, 1H).

### Example 23: (R)-1-(3-(5-(3-Hydroxy-1-methyl-2-oxopyrrolidin-3-yl)isoxazol-3-yl)phenyl)imidazo[1,5-a]pyridine-3-carboxamide

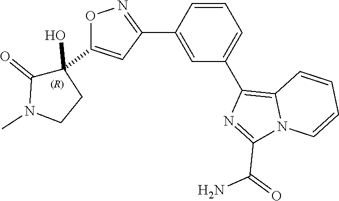

To a large vial was added (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 200 mg, 0.52 mmol), 1-bromoimidazo[1,5-a]pyridine-3-carboxamide (190 mg, 0.79 mmol) and Pd(PPh₃)₄ (61 mg, 0.05 mmol). Then, a mixture of 1,4-dioxane (20 mL) and 2 M K₂CO₃ (1.2 mL, 2.4 mmol) were degassed together with N₂ for 20 min and added to the reaction mixture. The vial was sealed and evacuated/purged with nitrogen 3 times, and then placed in an aluminum heating mantle at 100° C. After 2 h, the mixture was heated at 80° C. overnight. After 18 h, the reaction mixture was filtered through a diatomaceous earth pad (while still warm) and rinsed further with EtOAc and THF and the eluent was concentrated to give a tan viscous oil. The material was re-dissolved in CHCl₃ and purified by FCC with a gradient of 0-90% EtOAc in DCM to give (R)-1-(3-(5-(3-hydroxy-1-methyl-2-oxopyrrolidin-3-yl)isoxazol-3-yl)phenyl)imidazo[1,5-a]pyridine-3-carboxamide (151 mg, 70%) as an off-white fluffy solid. MS (ESI): Mass calcd. for C₁₉H₁₆N₆O₂, 417.4; m/z found, 418.1 [M+H]⁺. ¹H NMR (500 MHz, CD₃OD) δ 9.48 (dt, J=7.2, 1.1 Hz, 1H), 8.46-8.32 (m, 1H), 8.11-7.99 (m, 2H), 7.81-7.77 (m, 1H), 7.67-7.51 (m, 1H), 7.21-7.18 (m, 1H), 7.01-6.91 (m, 2H), 3.65-3.47 (m, 2H), 2.96 (s, 3H), 2.78-2.72 (m, 1H), 2.43-2.37 (m, 1H).

### Example 24: (R)-3-(3-(3-(1H-Pyrrolo[2,3-b]pyridin-3-yl)phenyl)isoxazol-5-yl)-3-hydroxy-1-methylpyrrolidin-2-one

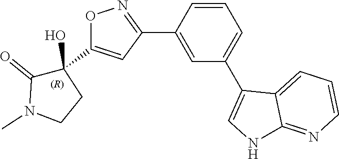

Step A: tert-Butyl (R)-3-(3-(5-(3-hydroxy-1-methyl-2-oxopyrrolidin-3-yl)isoxazol-3-yl)phenyl)-1H-pyrrolo[2,3-b]pyridine-1-carboxylate. To a solution of (R)-3-hydroxy-3-(3-(3-iodophenyl)isoxazol-5-yl)-1-methylpyrrolidin-2-one (Intermediate 23, 90 mg, 0.234 mmol) in 1,4-dioxane (10 mL) and water (1.5 mL) were added tert-butyl 3-(4,4,5,5-tetramethyl-1,3,2-dioxaborolan-2-yl)-1H-pyrrolo[2,3-b]pyridine-1-carboxylate (Intermediate 24, 604 mg, 0.70 mmol), bis(tri-tert-butylphosphine)palladium(0) (12 mg, 0.02 mmol) and K₃PO₄ (149 mg, 0.70 mmol). The mixture was heated at 75° C. for 16 h, then diluted with H₂O (20 mL) and extracted with EtOAc (3×15 mL). The combined organic solvent extracts were washed with brine (20 mL), dried over Na₂SO₄, filtered and concentrated to dryness. The residue was purified by preparative TLC (DCM/MeOH=20/1) to give tert-butyl (R)-3-(3-(5-(3-hydroxy-1-methyl-2-oxopyrrolidin-3-yl)isoxazol-3-yl)phenyl)-1H-pyrrolo[2,3-b]pyridine-1-carboxylate (50 mg, 0.086 mmol) as a yellow solid. MS (ESI): Mass calcd. for C₂₆H₂₆N₄O₅, 474.2; m/z found, 475.2 [M+H]⁺.

Step B: (R)-3-(3-(3-(1H-Pyrrolo[2,3-b]pyridin-3-yl)phenyl)isoxazol-5-yl)-3-hydroxy-1-methylpyrrolidin-2-one. A solution of tert-butyl (R)-3-(3-(5-(3-hydroxy-1-methyl-2-oxopyrrolidin-3-yl)isoxazol-3-yl)phenyl)-1H-pyrrolo[2,3-b]pyridine-1-carboxylate (50.0 mg, 0.11 mmol) in HCl solution (8 mL, 7 M in 1,4-dioxane) was stirred at 25° C. for 2 h, then concentrated to dryness. The residue was dissolved in H₂O (10 mL) and the pH of the solution was adjusted to pH>7 with saturated aqueous NaHCO₃. The solution was then extracted with DCM/i-PrOH (3×20 mL). The combined organic extracts were dried over Na₂SO₄, filtered, and concentrated. The residue was purified by preparative TLC (DCM/MeOH=20/1, R_{f}=0.35) to give (R)-3-(3-(3-(1H-pyrrolo[2,3-b]pyridin-3-yl)phenyl)isoxazol-5-yl)-3-hydroxy-1-methylpyrrolidin-2-one (9 mg, 22%) as a white solid. MS (ESI): Mass calcd. for C₂₁H₁₈N₄O₃, 374.1; m/z found, 375.1 [M+H]⁺. ¹H NMR (400 MHz, DMSO-d₆) δ 12.01 (s, 1H), 8.29-8.33 (m, 2H), 8.16 (s, 1H), 8.04 (s, 1H), 7.86 (d, J=7.6 Hz, 1H), 7.75 (d, J=7.6 Hz, 1H), 7.57 (t, J=8.0 Hz, 1H), 7.20-7.17 (m, 1H), 7.13 (s, 1H), 6.71 (s, 1H), 3.49-3.40 (m, 2H), 2.84 (s, 3H), 2.60-2.54 (m, 1H), 2.32-2.25 (m, 1H).

### Example 25: (R)-3-Hydroxy-1-methyl-3-(3-(3-(4-methylpyrido[3,2-d]pyrimidin-6-yl)phenyl)isoxazol-5-yl)pyrrolidin-2-one

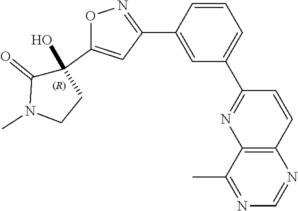

Step A: 6-Chloro-4-methylpyrido[3,2-d]pyrimidine. Trimethylaluminium (1.25 mL, 2 M in THF, 3.00 mmol) was added to a mixture of 4,6-dichloropyrido[3,2-d]pyrimidine (500 mg, 2.50 mmol) and THF (20 mL) that had been cooled to 0° C. The mixture was purged with N₂ for 5 minutes, treated with Pd(PPh₃)₄ (144 mg, 0.13 mmol), purged with N₂ for another 5 minutes, and then heated at 80° C. under a N₂ atmosphere for 8 h. The mixture was then cooled to room temperature, diluted with saturated aqueous NH₄Cl solution (20 mL), and extracted with ethyl acetate (100 mL). The organic phase was washed with brine (50 mL), dried over anhydrous Na₂SO₄, filtered, and concentrated under reduced pressure. The resulting residue was purified by FCC over silica gel (eluent: petroleum ether:ethyl acetate=1:0 to 3:1) to afford 6-chloro-4-methylpyrido[3,2-d]pyrimidine (270 mg, 60%) as a pale yellow solid. ¹H NMR (400 MHz, CDCl₃) δ 9.24 (s, 1H), 8.27 (d, J=8.8 Hz, 1H), 7.78 (d, J=8.8 Hz, 1H), 3.06 (s, 3H).

Step B: (R)-3-Hydroxy-1-methyl-3-(3-(3-(4-methylpyrido[3,2-d]pyrimidin-6-yl)phenyl)isoxazol-5-yl)pyrrolidin-2-one. A mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 360 mg, 0.94 mmol), 6-chloro-4-methylpyrido[3,2-d]pyrimidine (168 mg, 0.935 mmol), K₃PO₄ (597 mg, 2.81 mmol), 1,4-dioxane (4 mL), and H₂O (1 mL) was purged with Ar for 5 minutes, then treated with 1,1′-bis(di-tert butylphosphino)ferrocene palladium dichloride (61 mg, 0.09 mmol), and purged with Ar for another 5 minutes. The resultant mixture was subjected to microwave irradiation for 1 h at 90° C. The mixture was then cooled to room temperature and concentrated under reduced pressure. The product was purified by preparative HPLC using an HPLC column, such a Phenomenex Gemini-NX, 150 mm×30 mm×5 μm column (eluent: 27% to 51%, water (0.04% NH₄OH+10 mM NH₄HCO₃)—CH₃CN). The product was suspended in water (10 mL), the mixture was frozen by insertion into a −78° C. bath, and then lyophilized to dryness to afford (R)-3-hydroxy-1-methyl-3-(3-(3-(4-methylpyrido[3,2-d]pyrimidin-6-yl)phenyl)isoxazol-5-yl)pyrrolidin-2-one (99 mg, 26%) as a red solid. MS (ESI): Mass calcd. for C₂₂H₁₉N₅O₃, 401.2; m/z found, 402.0 [M+H]⁺. ¹H NMR (400 MHz, DMSO-d₆) δ 9.21 (s, 1H), 8.83-8.69 (m, 2H), 8.54-8.44 (m, 2H), 8.11-8.03 (m, 1H), 7.79-7.69 (m, 1H), 7.22 (s, 1H), 6.78 (s, 1H), 3.52-3.44 (m, 2H), 3.09 (s, 3H), 2.86 (s, 3H), 2.64-2.57 (m, 1H), 2.37-2.26 (m, 1H).

### Example 26: (R)-3-(3-(3-(4-Amino-8-methylpyrido[3,2-d]pyrimidin-6-yl)phenyl)isoxazol-5-yl)-3-hydroxy-1-methylpyrrolidin-2-one

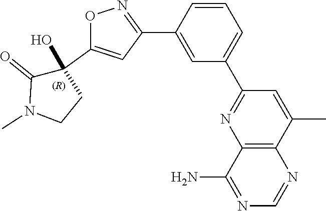

Step A: 3-Amino-4-bromo-6-chloropicolinonitrile. N-Bromosuccinimide (3.4 g, 19 mmol) was added to a solution of 3-amino-6-chloropicolinonitrile (2.7 g, 18 mmol) and DMF (50 mL). The resultant mixture was heated at 90° C. for 2 h. The mixture was then cooled to room temperature, treated with saturated aqueous Na₂SO₃ solution (100 mL) and stirred for 1 h. The resultant mixture was treated with saturated aqueous NaHCO₃ solution (100 mL) and extracted with ethyl acetate (40 mL×3). The combined organic extracts were washed with brine (10 mL), dried over anhydrous Na₂SO₄, filtered, and concentrated to dryness under reduced pressure. The resulting residue was purified by FCC (eluent: petroleum ether:ethyl acetate=1:0 to 1:1) to afford 3-amino-4-bromo-6-chloropicolinonitrile (1.3 g, 30%) as a yellow solid. MS (ESI): Mass calcd. for C₆H₃BrClN₃ 230.9; m/z found 233.7 [M+H]⁺.

Step B: 3-Amino-6-chloro-4-methylpicolinonitrile. 3-Amino-4-bromo-6-chloropicolinonitrile (1.2 g, 5.2 mmol), 2,4,6-trimethyl-1,3,5,2,4,6-trioxatriborinane (0.8 mL, 5.7 mmol), K₂CO₃ (13 mL, 2.0 M in water, 26 mmol), and 1,4-dioxane (30 mL) were added to a 100 mL round bottom flask. The mixture was sparged with Ar for 5 minutes and then treated with PdCl₂(dppf)·CH₂Cl₂ (0.5 g, 0.5 mmol). The mixture was sparged with Ar for another 5 minutes and the resultant mixture was heated at 80° C. for 2 h. The mixture was then cooled to room temperature, diluted with H₂O (100 mL), and extracted with ethyl acetate (60 mL×3). The combined organic extracts were washed with brine (10 mL), dried with anhydrous Na₂SO₄, filtered, and concentrated to dryness under reduced pressure. The resulting residue was purified by FCC (eluent: petroleum ether:ethyl acetate=5:1 to 1:1) to afford 3-amino-6-chloro-4-methylpicolinonitrile (600 mg, 69%) as a yellow solid.

Step C: 6-Chloro-8-methylpyrido[3,2-d]pyrimidin-4-amine. 3-Amino-6-chloro-4-methylpicolinonitrile (1.05 g, 6.27 mmol), formimidamide acetate (5.22 g, 50.1 mmol), K₃PO₄ (13.3 g, 62.7 mmol), and 1,4-dioxane (30 mL) were added to 100 mL round-bottomed flask. The reaction mixture was heated at 90° C. for 2 h. The mixture was then cooled to room temperature, diluted with H₂O (100 mL), and extracted with ethyl acetate (60 mL×3). The combined organic extracts were washed with brine (10 mL), dried over anhydrous Na₂SO₄, filtered, and concentrated to dryness under reduced pressure to give the product which was purified by FCC (eluent: petroleum ether:ethyl acetate=1:0 to 1:1) to afford 6-chloro-8-methylpyrido[3,2-d]pyrimidin-4-amine (1.0 g, 82%) as a yellow solid. MS (ESI): Mass calcd. for C₈H₇ClN₄, 194.0; m/z found, 195.1 [M+H]⁺.

Step D: (R)-3-(3-(3-(4-Amino-8-methylpyrido[3,2-d]pyrimidin-6-yl)phenyl)isoxazol-5-yl)-3-hydroxy-1-methylpyrrolidin-2-one. A mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 250 mg, 0.651 mmol), 6-chloro-8-methylpyrido[3,2-d]pyrimidin-4-amine (152 mg, 0.781 mmol), K₃PO₄ (414 mg, 1.95 mmol), 1,4-dioxane (4 mL), and H₂O (1 mL) was sparged with Ar for 5 minutes, treated with 1,1′-bis(di-tert butylphosphino)ferrocene palladium dichloride (42 mg, 0.064 mmol), and sparged with Ar for another 5 minutes. The resultant mixture was subjected to microwave irradiation at 90° C. for 1 h. The resulting mixture was then cooled to room temperature and concentrated under reduced pressure. The resulting residue was purified by preparative HPLC using an HPLC column, such an Welch Xtimate C18, 150 mm×25 mm×5 μm column (eluent: 20% to 50%, water (0.04% NH₄OH)—CH₃CN) to afford the product. The product was suspended in water (10 mL), the mixture was frozen by insertion into a −78° C. bath, and then lyophilized to dryness to afford (R)-3-(3-(3-(4-amino-8-methylpyrido[3,2-d]pyrimidin-6-yl)phenyl)isoxazol-5-yl)-3-hydroxy-1-methylpyrrolidin-2-one (100 mg, 35%) as a yellow solid. MS (ESI): Mass calcd. for C₂₂H₂₀N₆O₃, 416.2; m/z found, 417.1 [M+H]⁺. ¹H NMR (400 MHz, DMSO-d₆) δ 8.73 (s, 1H), 8.57 (d, J=7.9 Hz, 1H), 8.50-8.39 (m, 2H), 8.17 (br s, 1H), 8.06-7.88 (m, 2H), 7.72-7.61 (m, 1H), 7.31 (s, 1H), 6.99-6.50 (m, 1H), 3.52-3.40 (m, 2H), 2.85 (s, 3H), 2.68 (s, 3H), 2.62-2.55 (m, 1H), 2.36-2.22 (m, 1H).

### Example 27: (S)-3-(3-(3-(4-Amino-8-methylpyrido[3,2-d]pyrimidin-6-yl)phenyl)isoxazol-5-yl)-3-hydroxy-1-methylpyrrolidin-2-one

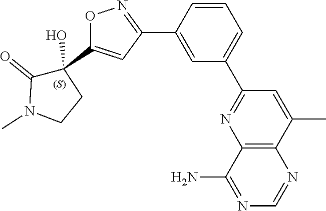

Step A: (S)-3-Hydroxy-1-methyl-3-(3-(3-(4,4,5,5-tetramethyl-1,3,2-dioxaborolan-2-yl)phenyl)isoxazol-5-yl)pyrrolidin-2-one. 4,4,4′,4′,5,5,5′,5′-Octamethyl-2,2′-bi(1,3,2-dioxaborolane) (300 mg, 1.18 mmol) was added to (S)-3-(3-(3-bromophenyl)isoxazol-5-yl)-3-hydroxy-1-methylpyrrolidin-2-one (Intermediate 26, 200 mg, 0.6 mmol) and KOAc (380 mg, 1.79 mmol) in 1,4-dioxane (5 mL). The resultant mixture was sparged with Ar for 5 minutes and then treated with PdCl₂(dppf) (43.0 mg, 0.06 mmol) to provide a reaction mixture. The reaction mixture was sparged with Ar for another 5 minutes and then subjected to microwave irradiation at 100° C. for 1 h. After this time, the reaction mixture was cooled to room temperature, and filtered through a pad of diatomaceous earth. The filtrate was concentrated to dryness under reduced pressure to afford the product, which was purified by FCC (eluent: petroleum ether:ethyl acetate=1:0 to 0:1) to afford (S)-3-hydroxy-1-methyl-3-(3-(3-(4,4,5,5-tetramethyl-1,3,2-dioxaborolan-2-yl)phenyl)isoxazol-5-yl)pyrrolidin-2-one (156 mg, 62%) as a yellow oil. MS (ESI): Mass calcd. for C₂₀H₂₅BN₂O₅, 384.2; m/z found, 385.2 [M+H]⁺.

Step B: (S)-3-(3-(3-(4-Amino-8-methylpyrido[3,2-d]pyrimidin-6-yl)phenyl)isoxazol-5-yl)-3-hydroxy-1-methylpyrrolidin-2-one. 6-Chloro-8-methylpyrido[3,2-d]pyrimidin-4-amine (Example 26 Step C, 51 mg, 0.26 mmol), (S)-3-hydroxy-1-methyl-3-(3-(3-(4,4,5,5-tetramethyl-1,3,2-dioxaborolan-2-yl)phenyl)isoxazol-5-yl)pyrrolidin-2-one (100 mg, 0.26 mmol), K₃PO₄ (166 mg, 0.78 mmol), 1,4-dioxane (2 mL), and H₂O (0.5 mL) were added to a 5 mL microwave tube. The resultant mixture was sparged with Ar for 5 minutes, treated with Pd(dtbpf)Cl₂ (17 mg, 0.03 mmol), and sparged with Ar for another 5 minutes to provide a reaction mixture. This reaction mixture was subjected to microwave irradiation at 90° C. for 1 h. After this time, the reaction mixture was cooled to room temperature and filtered through a pad of diatomaceous earth. The filtrate was concentrated to dryness under reduced pressure to afford the product, which was purified by preparative HPLC using a HPLC column such as a Welch Xtimate C18 150×25 mm×5 μm column (eluent: 23% to 43% CH₃CN and H₂O with (0.04% NH₄OH+10 mM NH₄HCO₃)), and further purified by SFC using an SFC column, such as a DAICEL CHIRALPAK® AS 250 mm×30 mm, 10 μm column (isocratic elution: EtOH (containing 0.1% of 25% NH₃): supercritical CO₂, 45%: 55%). The product containing fractions were collected and concentrated under reduced pressure. The product was suspended in water (10 mL), the mixture was frozen by insertion into a −78° C. bath, and then lyophilized to dryness to afford (S)-3-(3-(3-(4-amino-8-methylpyrido[3,2-d]pyrimidin-6-yl)phenyl)isoxazol-5-yl)-3-hydroxy-1-methylpyrrolidin-2-one (36.1 mg, 33%) as a colorless solid. MS (ESI): Mass calcd. for C₂₂H₂₀N₆O₃, 416.2; m/z found, 417.1 [M+H]⁺. ¹H NMR (400 MHz, DMSO-d₆) δ 8.70 (s, 1H), 8.53 (d, J=7.8 Hz, 1H), 8.47-8.37 (m, 2H), 8.13 (br s, 1H), 8.02-7.85 (m, 2H), 7.68-7.57 (m, 1H), 7.26 (s, 1H), 6.72 (br s, 1H), 3.47-3.37 (m, 2H), 2.82 (s, 3H), 2.64 (s, 3H), 2.57-2.52 (m, 1H), 2.30-2.22 (m, 1H).

### Example 28: (R)-6-(3-(2-(3-Hydroxy-1-methyl-2-oxopyrrolidin-3-yl)thiazol-5-yl)phenyl)picolinamide

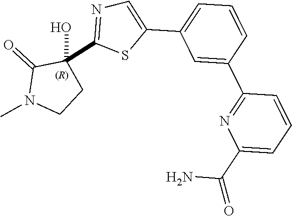

Step A: 5-(3-Bromophenyl)thiazole. Tricyclohexylphosphine tetrafluoroborate (4.3 g, 12 mmol) was added to a mixture of thiazole (5.0 g, 59 mmol), 1,3-dibromobenzene (10.7 mL, 88.1 mmol), PivOH (1.80 g, 17.6 mmol), K₂CO₃ (12.2 g, 88.1 mmol), and DMA (100 mL). The mixture was purged with Ar three times and then treated with Pd(OAc)₂ (1.32 mg, 5.87 mmol). The resultant mixture was heated at 100° C. for 2 h. After this time, the mixture was diluted with water (200 mL) and extracted with ethyl acetate (200 mL×3). The combined organic extracts were washed with brine, dried over Na₂SO₄, filtered, and concentrated to afford the product, which was purified by FCC (eluent: petroleum ether:ethyl acetate=1:0 to 10:1) to provide 5-(3-bromophenyl)thiazole (1.9 g, 13%) as an orange oil. MS (ESI): Mass calcd. for C₉H₆BrNS 240.1; m/z found, 242.0 [M+H]⁺.

Step B: (R,S)-3-(5-(3-Bromophenyl)thiazol-2-yl)-3-hydroxy-1-methylpyrrolidin-2-one. Lithium diisopropylamide (5.94 mL, 11.9 mmol, 2.0 M in hexanes) was added to a solution of 5-(3-bromophenyl)thiazole (1.9 g, 7.9 mmol) in THF (10 mL) that had been cooled to −72° C. The resultant mixture was stirred at −72° C. for 30 minutes, treated with a solution of 1-methylpyrrolidine-2,3-dione (1.07 g, 9.50 mmol) in THF (10 mL), and stirred at −72° C. for 1 hour. After this time, the reaction mixture was quenched with H₂O (50 mL) and extracted with DCM/MeOH (10:1) (50 mL×3). The combined organic extracts were washed with brine, dried over anhydrous Na₂SO₄, filtered, and concentrated to dryness under reduced pressure. The resulting residue was purified by FCC (eluent: methylene chloride:methanol=1:0 to 10:1) to afford (R,S)-3-(5-(3-bromophenyl)thiazol-2-yl)-3-hydroxy-1-methylpyrrolidin-2-one (741 mg, 25%) as a brown oil. MS (ESI): Mass calcd. for C₁₄H₁₃BrN₂O₂S, 350.0; m/z found, 353.1 [M+H]⁺.

Step C: (R,S)-3-Hydroxy-1-methyl-3-(5-(3-(4,4,5,5-tetramethyl-1,3,2-dioxaborolan-2-yl)phenyl)thiazol-2-yl)pyrrolidin-2-one. (R,S)-3-(5-(3-Bromophenyl)thiazol-2-yl)-3-hydroxy-1-methylpyrrolidin-2-one (641 mg, 1.82 mmol), 4,4,4′,4′,5,5,5′,5′-octamethyl-2,2′-bi(1,3,2-dioxaborolane) (691 mg, 2.72 mmol), and KOAc (534 mg, 5.44 mmol) and 1,4-dioxane (5 mL) were added to a microwave tube. The resultant mixture was purged with Ar for 5 minutes and then treated with Pd(dppf)Cl₂ (133 mg, 0.181 mmol). The mixture was purged with Ar for another 5 minutes and then subjected to microwave irradiation at 90° C. for 1 h. After this time, the mixture was poured into H₂O (15 mL), and extracted with EtOAc (15 mL×2). The combined organic extracts were washed with brine (10 mL), dried over anhydrous Na₂SO₄, filtered, and concentrated to dryness under reduced pressure. The resulting residue was purified by FCC (eluent: petroleum ether:ethyl acetate=1:0 to 1:2) to afford (R,S)-3-hydroxy-1-methyl-3-(5-(3-(4,4,5,5-tetramethyl-1,3,2-dioxaborolan-2-yl)phenyl)thiazol-2-yl)pyrrolidin-2-one (425 mg, 48%) as a brown solid. MS (ESI): Mass calcd. for C₂₀H₂₅BN₂O₄S 400.3; m/z found, 401.3 [M+H]⁺.

Step D: (R,S)-6-(3-(2-(3-Hydroxy-1-methyl-2-oxopyrrolidin-3-yl)thiazol-5-yl)phenyl)picolinamide. 6-Chloropicolinamide (191 mg, 1.22 mmol), (R,S)-3-hydroxy-1-methyl-3-(5-(3-(4,4,5,5-tetramethyl-1,3,2-dioxaborolan-2-yl)phenyl)thiazol-2-yl)pyrrolidin-2-one (325 mg, 0.812 mmol), and K₃PO₄ (517 mg, 2.44 mmol) were added to a microwave tube and the resultant mixture dissolved in 1,4-dioxane (1.6 mL) and H₂O (0.4 mL). The resultant mixture was purged with Ar for 5 minutes and then treated with Pd(dtbpf)Cl₂ (53 mg, 0.081 mmol). The resultant mixture was purged with Ar for another 5 minutes and then subjected to microwave irradiation at 100° C. for 1 hour.

After this time, the mixture was poured into H₂O (10 mL) and extracted with EtOAc (10 mL×2). The combined organic extracts were washed with brine (10 mL), dried over anhydrous Na₂SO₄, filtered, and concentrated to dryness under reduced pressure to give the product which was purified by FCC (eluent: methylene chloride:methanol=1:0 to 10:1) to afford (R,S)-6-(3-(2-(3-hydroxy-1-methyl-2-oxopyrrolidin-3-yl)thiazol-5-yl)phenyl)picolinamide (200 mg, 52%) as a brown solid. MS (ESI): Mass calcd. for C₂₀H₁₈N₄O₃S 394.1; m/z found 395.1 [M+H]⁺.

Step E: (R)-6-(3-(2-(3-Hydroxy-1-methyl-2-oxopyrrolidin-3-yl)thiazol-5-yl)phenyl)picolinamide. The (R) and (S) enantiomers of (R,S)-6-(3-(2-(3-Hydroxy-1-methyl-2-oxopyrrolidin-3-yl)thiazol-5-yl)phenyl)picolinamide (300 mg, 0.76 mmol) were separated by SFC using an SFC column, such as a DAICEL CHIRALCEL® OD 250 mm×30 mm, 10 μm column (isocratic elution: EtOH (containing 0.1% of 25% aqueous NH₃ solution): supercritical CO₂, 45%: 55%). The second eluting product was (R)-6-(3-(2-(3-hydroxy-1-methyl-2-oxopyrrolidin-3-yl)thiazol-5-yl)phenyl)picolinamide. The fractions containing this enantiomer were collected and concentrated under reduced pressure. The product was suspended in water (10 mL), the mixture was frozen by insertion into a −78° C. bath, and then lyophilized to dryness to afford (R)-6-(3-(2-(3-hydroxy-1-methyl-2-oxopyrrolidin-3-yl)thiazol-5-yl)phenyl)picolinamide, which was further purified with preparative HPLC using an HPLC column, such a Phenomenex Gemini NX—C18 75×30 mm×3 μm column (eluent: 25% to 55%, CH₃CN and H₂O (with 0.05% NH₄OH and 10 mM NH₄HCO₃)). The product was suspended in water (10 mL), the mixture was frozen by insertion into a −72° C. bath, and then lyophilized to dryness to afford (R)-6-(3-(2-(3-hydroxy-1-methyl-2-oxopyrrolidin-3-yl)thiazol-5-yl)phenyl)picolinamide (48 mg, 16%) as a colorless solid. MS (ESI): Mass calcd. for C₂₀H₁₈N₄O₃S, 394.1; m/z found, 395.0 [M+H]⁺. ¹H NMR (400 MHz, DMSO-d₆) δ 8.54-8.41 (m, 2H), 8.33-8.27 (m, 3H), 8.15-8.02 (m, 2H), 7.80-7.71 (m, 2H), 7.64-7.56 (m, 1H), 6.99 (s, 1H), 3.57-3.52 (m, 2H), 2.84 (s, 3H), 2.79-2.71 (m, 1H), 2.34-2.24 (m, 1H).

### Example 29: (R)-6-(3-(4-(3-Hydroxy-1-methyl-2-oxopyrrolidin-3-yl)-1H-pyrazol-1-yl)phenyl)picolinamide

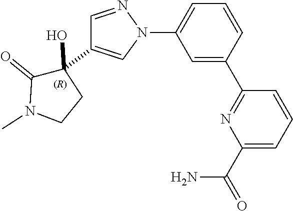

Step A: 4-Bromo-1-((2-(trimethylsilyl)ethoxy)methyl)-1H-pyrazole. Sodium hydride (5.99 g, 60% in mineral oil, 150 mmol) was added to a solution of 4-bromo-1H-pyrazole (20.0 g, 136 mmol) and THF (100 mL) that had been cooled to 0° C. The resultant mixture was stirred at 0° C. for 1 h, treated with 2-(trimethylsilyl)ethoxymethyl chloride (27.2 g, 163 mmol) dropwise, and stirred for 16 h with gradual warming to room temperature. The resulting mixture was poured into a saturated ammonium chloride solution (200 mL) and extracted with ethyl acetate (200 mL×3). The combined organic extracts were washed with brine (300 mL), dried over anhydrous Na₂SO₄, filtered, and concentrated to afford the product, which was purified by FCC (eluent: petroleum ether:methylene chloride=3:1 to 1:1) to afford 4-bromo-1-((2-(trimethylsilyl)ethoxy)methyl)-1H-pyrazole (32 g, 82%) as a colorless oil. ¹H NMR (400 MHz, CDCl₃) δ 7.61 (s, 1H), 7.51 (s, 1H), 5.40 (s, 2H), 3.61-3.52 (m, 2H), 0.95-0.90 (m, 2H), 0.00 (s, 9H).

Step B: (R,S)-3-Hydroxy-1-methyl-3-(1-((2-(trimethylsilyl)ethoxy)methyl)-1H-pyrazol-4-yl)pyrrolidin-2-one. n-Butyllithium (8.7 mL, 2.5 M in hexanes, 1.4 mmol) was added dropwise to a solution of 4-bromo-1-((2-(trimethylsilyl)ethoxy)methyl)-1H-pyrazole (5.0 g, 18 mmol) in anhydrous THF (50.0 mL) that had been cooled to −72° C. The resultant mixture was stirred at −72° C. for 30 minutes before it was treated with 1-methylpyrrolidine-2,3-dione (4.1 g, 36 mmol). The resultant mixture was stirred at −72° C. for 5 minutes, and then stirred for 15 minutes with gradual warming to room temperature. After this time, the mixture was poured into a saturated ammonium chloride solution (50 mL) and extracted with ethyl acetate (50 mL×3). The combined organic extracts were washed with brine (100 mL), dried over anhydrous Na₂SO₄, filtered, and concentrated. The product was purified by preparative HPLC using an HPLC column, such an Xtimate C18 150×40 mm, 10 μm column (eluent: 25% to 55%, water (0.04% NH₄OH+10 mM NH₄HCO₃)—CH₃CN). The fractions containing the product were collected and concentrated under vacuum. The residue was suspended in water (10 mL), the mixture was frozen by insertion into a −78° C. bath, and then lyophilized to dryness to afford (R,S)-3-hydroxy-1-methyl-3-(1-((2-(trimethylsilyl)ethoxy)methyl)-1H-pyrazol-4-yl)pyrrolidin-2-one (720 mg, 13%) as a yellow oil. MS (ESI): Mass calcd. for C₁₄H₂₅N₃O₃Si, 311.2; m/z found, 312.2 [M+H]⁺.

Step C: (R,S)-3-Hydroxy-1-methyl-3-(1H-pyrazol-4-yl)pyrrolidin-2-one. Tetrabutylammonium fluoride (11.6 mL, 1.0 M in THF, 11.6 mmol) was added to a mixture of (R,S)-3-hydroxy-1-methyl-3-(1-((2-(trimethylsilyl)ethoxy)methyl)-1H-pyrazol-4-yl)pyrrolidin-2-one (0.7 g, 2.3 mmol) and THF (10.0 mL) at room temperature. The resultant reaction mixture was heated at 90° C. for 16 h. The mixture was then concentrated under reduced pressure to afford the product, which was purified by FCC (eluent: methylene chloride:methanol=1:0 to 9:1) to afford (R,S)-3-hydroxy-1-methyl-3-(1H-pyrazol-4-yl)pyrrolidin-2-one (418 mg, 88%) as a yellow oil. MS (ESI): Mass calcd. for C₈H₁₁N₃O₂ 181.2; m/z found, 182.2 [M+H]⁺.

Step D: (R,S)-3-(1-(3-Bromophenyl)-1H-pyrazol-4-yl)-3-hydroxy-1-methylpyrrolidin-2-one. Pyridine (365 mg, 4.61 mmol) was added to a mixture of (R,S)-3-hydroxy-1-methyl-3-(1H-pyrazol-4-yl)pyrrolidin-2-one (418 mg, 2.31 mmol), (3-bromophenyl)boronic acid (927 mg, 4.61 mmol), copper diacetate (419 mg, 2.31 mmol), and DMF (10 mL). The resultant mixture was heated at 90° C. for 5 hours under an air atmosphere. The mixture was then poured into water (30 mL), and extracted with ethyl acetate (30 mL×3). The combined organic extracts were washed with brine (60 mL), dried over anhydrous Na₂SO₄, filtered, and concentrated to dryness. The product was purified by FCC (eluent: petroleum ether:ethyl acetate=1:0 to 0:1) to yield (R,S)-3-(1-(3-bromophenyl)-1H-pyrazol-4-yl)-3-hydroxy-1-methylpyrrolidin-2-one (120 mg, 15%) as a colorless oil. MS (ESI): Mass calcd. for C₁₄H₁₄BrN₃O₂ 336.2 m/z found, 337.1 [M+H]⁺.

Step E: (R,S)-3-Hydroxy-1-methyl-3-(1-(3-(4,4,5,5-tetramethyl-1,3,2-dioxaborolan-2-yl)phenyl)-1H-pyrazol-4-yl)pyrrolidin-2-one. [1,1′-bis(Diphenylphosphino)ferrocene]dichloropalladium(II) (65 mg, 0.089 mmol) was added to a mixture of (R,S)-3-(1-(3-bromophenyl)-1H-pyrazol-4-yl)-3-hydroxy-1-methylpyrrolidin-2-one (300 mg, 0.9 mmol), bis(pinacolato)diboron (272 mg, 1.07 mmol), KOAc (175 mg, 1.79 mmol), and 1,4-dioxane (5 mL) under a N₂ atmosphere. The resultant mixture was subjected to microwave irradiation at 90° C. for 2 h. The mixture was then poured into water (30 mL) and extracted with ethyl acetate (30 mL×3). The combined organic extracts were washed with brine (60 mL), dried over anhydrous Na₂SO₄, filtered, and concentrated to dryness under reduced pressure. The product was purified by FCC (eluent: petroleum ether:ethyl acetate=1:0 to 0:1) to afford (R,S)-3-hydroxy-1-methyl-3-(1-(3-(4,4,5,5-tetramethyl-1,3,2-dioxaborolan-2-yl)phenyl)-1H-pyrazol-4-yl)pyrrolidin-2-one (200 mg) as a colorless oil. MS (ESI): Mass calcd. for C₂₀H₂₆BN₃O₄, 383.2; m/z found, 384.3 [M+H]⁺.

Step F: (R,S)-6-(3-(4-(3-Hydroxy-1-methyl-2-oxopyrrolidin-3-yl)-1H-pyrazol-1-yl)phenyl)picolinamide. (R,S)-3-Hydroxy-1-methyl-3-(1-(3-(4,4,5,5-tetramethyl-1,3,2-dioxaborolan-2-yl)phenyl)-1H-pyrazol-4-yl)pyrrolidin-2-one (180 mg, 0.47 mmol), 6-chloropicolinamide (110 mg, 0.705 mmol) and 1,4-dioxane/H₂O (10 mL, 4/1) were added to a microwave tube. The mixture was sparged with N₂ for 5 minutes. [1,1′-bis(Diphenylphosphino)ferrocene]dichloropalladium(II) (31 mg, 0.047 mmol) and K₃PO₄ (299 mg, 1.41 mmol) were added and the mixture was sparged with N₂ for another 5 minutes. The resultant mixture was subjected to microwave irradiation at 100° C. for 1 hour. The mixture was then poured into water (30 mL) and extracted with ethyl acetate (30 mL×3). The combined organic extracts were washed with brine (60 mL), dried over anhydrous Na₂SO₄, filtered, and concentrated to dryness. The product was initially purified by FCC (eluent: petroleum ether:ethyl acetate=1:0 to 0:1), and then further purified by preparative HPLC using an HPLC column, such as a Phenomenex Gemini NX—C18 75×30 mm, 3 μm column (eluent: 15% to 45%, water (0.04% NH₄OH+10 mM NH₄HCO₃)—CH₃CN). The fractions containing the product were collected and concentrated under vacuum. The residue was suspended in water (10 mL), the mixture was frozen by insertion into a −78° C. bath, and then lyophilized to dryness to afford (R,S)-6-(3-(4-(3-hydroxy-1-methyl-2-oxopyrrolidin-3-yl)-1H-pyrazol-1-yl)phenyl)picolinamide (70 mg, 39%) as a colorless solid. MS (ESI): Mass calcd. for C₂₀H₁₉N₅O₃, 377.4; m/z found, 378.3 [M+H]⁺.

Step G: (R)-6-(3-(4-(3-Hydroxy-1-methyl-2-oxopyrrolidin-3-yl)-1H-pyrazol-1-yl)phenyl)picolinamide. The (R) and (S) enantiomers of (R,S)-6-(3-(4-(3-Hydroxy-1-methyl-2-oxopyrrolidin-3-yl)-1H-pyrazol-1-yl)phenyl)picolinamide (70.0 mg, 0.185 mmol) were separated by SFC using an SFC column, such as a DAICEL CHIRALPAK® AS 250 mm×30 mm, 10 μm column (isocratic elution: EtOH (containing 0.1% of 25% aqueous NH₃ solution): supercritical CO₂, 50%: 50%). The second eluting compound was (R)-6-(3-(4-(3-hydroxy-1-methyl-2-oxopyrrolidin-3-yl)-1H-pyrazol-1-yl)phenyl)picolinamide. The fractions containing the product were collected and concentrated under reduced pressure. The product was suspended in water (10 mL), the mixture was frozen by insertion into a −78° C. bath, and then lyophilized to dryness to afford (R)-6-(3-(4-(3-hydroxy-1-methyl-2-oxopyrrolidin-3-yl)-1H-pyrazol-1-yl)phenyl)picolinamide (22.3 mg, 32%) as a colorless solid. MS (ESI): Mass calcd. for C₂₀H₁₉N₅O₃ 377.4 m/z found, 378.0 [M+H]⁺. ¹H NMR (400 MHz, DMSO-d₆) δ 8.66 (s, 1H), 8.59-8.56 (m, 1H), 8.42 (s, 1H), 8.33-8.29 (m, 1H), 8.27-8.22 (m, 1H), 8.15-8.08 (m, 1H), 8.06-8.00 (m, 1H), 7.98-7.91 (m, 1H), 7.81-7.73 (m, 2H), 7.66-7.58 (m, 1H), 6.34-5.33 (m, 1H), 3.46-3.40 (m, 2H), 2.80 (s, 3H), 2.57-2.53 (m, 1H), 2.29-2.22 (m, 1H).

### Example 30: (R)-6-(3-(3-(3-Hydroxy-1-methyl-2-oxopyrrolidin-3-yl)-1-methyl-1H-pyrazol-5-yl)phenyl)picolinamide

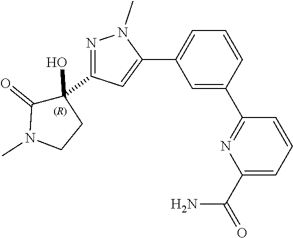

Step A: 3,5-Dibromo-1-methyl-1H-pyrazole. Sodium hydride (2.5 g, 60% purity, 63 mmol) was added to a solution of 3,5-dibromo-1H-pyrazole (7.0 g, 31 mmol) in THF (80 mL) that had been cooled to 0° C. under a N₂ atmosphere. The reaction mixture was stirred at 0° C. for 1 hour before it was treated with methyl iodide (11 g, 75 mmol) dropwise. The resultant mixture was stirred for 2 h and was gradually warmed to room temperature. The reaction mixture was then quenched with saturated aqueous NH₄Cl solution (100 mL) and extracted with ethyl acetate (200 mL×3). The combined organic extracts were washed with brine (30 mL), dried over anhydrous Na₂SO₄, filtered, and concentrated to dryness under reduced pressure. The product was purified by FCC (eluent: petroleum ether:ethyl acetate=1:0 to 1:1) to afford 3,5-dibromo-1-methyl-1H-pyrazole (7.0 g, 94%) as a yellow oil. MS (ESI): Mass calcd. for C₄H₄Br₂N₂, 237.9; m/z found 238.9 [M+H]⁺. ¹H NMR (400 MHz, CDCl₃) δ 6.35-6.29 (m, 1H), 3.87 (s, 3H), 6.35-6.29 (m, 1H), 3.87 (s, 3H).

Step B: 3-Bromo-5-(3-chlorophenyl)-1-methyl-1H-pyrazole. 3,5-Dibromo-1-methyl-1H-pyrazole (6.0 g, 25 mmol), (3-chlorophenyl)boronic acid (3.32 g, 21.2 mmol), and K₂CO₃ (10.4 g, 75.3 mmol) were added to a solution of 1,4-dioxane (80 mL) and H₂O (20 mL). The mixture was sparged with Ar for 5 minutes and then treated with Pd(dppf)Cl₂ (1.83 g, 2.50 mmol). The mixture was sparged with Ar for another 5 minutes and heated at 105° C. for 16 h. This reaction mixture combined with another reaction mixture batch of 3-bromo-5-(3-chlorophenyl)-1-methyl-1H-pyrazole (200 mg, 0.5 mmol). These combined batches were then poured into H₂O (30 mL) and extracted with ethyl acetate (30 mL×3). The organic solvent extracts were washed with brine (10 mL), dried over anhydrous Na₂SO₄, filtered, and concentrated to dryness. The product was initially purified by FCC (eluent: petroleum ether:ethyl acetate=1:0 to 3:1). The product was further purified by preparative HPLC using an HPLC column, such as a YMC-Triart Prep C18 150×40 mm×7 μm column (eluent: 47% to 77%, CH₃CN and H₂O with (0.04% NH₄OH+10 mM NH₄HCO₃)) to afford 3-bromo-5-(3-chlorophenyl)-1-methyl-1H-pyrazole (2.5 g) as a white solid. MS (ESI): Mass calcd. for C₁₀H₈BrClN₂, 270.0; m/z found 273.0 [M+H]⁺. ¹H NMR (400 MHz, DMSO-d₆) δ 7.67-7.64 (m, 1H), 7.58-7.52 (m, 3H), 6.66 (s, 1H), 3.83 (s, 3H).

Step C: (R,S)-3-(5-(3-Chlorophenyl)-1-methyl-1H-pyrazol-3-yl)-3-hydroxy-1-methylpyrrolidin-2-one. n-Butyllithium (3.2 mL, 8.0 mmol, 2.5 M in hexanes) was added dropwise to a solution of 3-bromo-5-(3-chlorophenyl)-1-methyl-1H-pyrazole (2.00 g, 7.36 mmol) and DCM (30 mL) that had been cooled to −72° C., under a N₂ atmosphere.

The resultant mixture was stirred at −72° C. for 0.5 hours before treating with a solution of 1-methylpyrrolidine-2,3-dione (1.00 g, 8.84 mmol) and DCM (10 mL) under a N₂ atmosphere. The mixture was stirred at −72° C. for 1 hour under a N₂ atmosphere. The reaction mixture was then quenched with saturated aqueous NH₄Cl solution (50 mL) and extracted with DCM (100 mL×3). The combined organic extracts were dried over anhydrous Na₂SO₄, filtered, and concentrated to dryness. The product was purified by FCC. The initial eluent was a gradient of 100% petroleum ether/0% EtOAc to 0% petroleum ether/100% EtOAc, and then the eluent was changed to 100% EtOAc/0% MeOH to 80% EtOAc/20% MeOH to afford 3-(5-(3-chlorophenyl)-1-methyl-1H-pyrazol-3-yl)-3-hydroxy-1-methylpyrrolidin-2-one (1.2 g, 45%) as a yellow solid. MS (ESI): Mass calcd. for C₁₅H₁₆ClN₃O₂, 305.1; m/z found 306.1 [M+H]⁺.

Step D: (R,S)-3-Hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one. Chloro(2-dicyclohexylphosphino-2′,4′,6′-triisopropyl-1,1′-biphenyl)[2-(2′-amino-1,1′-biphenyl)]palladium(II) (267 mg, 0.339 mmol) was added to a mixture of 3-(5-(3-chlorophenyl)-1-methyl-1H-pyrazol-3-yl)-3-hydroxy-1-methylpyrrolidin-2-one (1.00 g, 3.27 mmol), 4,4,4′,4′,5,5,5′,5′-octamethyl-2,2′-bi(1,3,2-dioxaborolane) (1.67 g, 6.58 mmol), KOAc (966 mg, 9.84 mmol), and 1,4-dioxane (60 mL) under a N₂ atmosphere. The mixture was heated at 110° C. for 6 h under a N₂ atmosphere. The reaction mixture was concentrated to dryness. The resulting residue was purified by FCC using 100% petroleum ether/0% EtOAc to 0% petroleum ether/100% EtOAc. The eluent was then changed to 0% MeOH/100% EtOAc to 20% MeOH/80% EtOAc to afford (R,S)-3-hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one (1.2 g) as a brown oil. MS (ESI): Mass calcd. for C₂₁H₂₈BN₃O₄, 397.2; m/z found 398.1 [M+H]⁺.

Step E: (R,S)-6-(3-(3-(3-Hydroxy-1-methyl-2-oxopyrrolidin-3-yl)-1-methyl-1H-pyrazol-5-yl)phenyl)picolinamide. [1,1′-Bis(di-tert-butylphosphino)ferrocene]dichloropalladium(II) (197 mg, 0.302 mmol) was added to a mixture of (R,S)-3-hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one (1.2 g, 3.02 mmol), 6-chloropicolinamide (525 mg, 3.02 mmol), K₃PO₄ (1.90 g, 8.95 mmol), 1,4-dioxane (50 mL), and H₂O (12.5 mL) under a N₂ atmosphere. The mixture was heated at 100° C. for 16 h under a N₂ atmosphere. The combined mixture was concentrated to dryness under reduced pressure to give the product, which was purified by FCC (eluent: petroleum ether:ethyl acetate=1:0 to 0:1, then ethyl acetate:methanol=1:0 to 5:1) to afford (R,S)-6-(3-(3-(3-hydroxy-1-methyl-2-oxopyrrolidin-3-yl)-1-methyl-1H-pyrazol-5-yl)phenyl)picolinamide (800 mg) as a brown solid. MS (ESI): Mass calcd. for C₂₁H₂₁N₅O₃, 391.2; m/z found 392.2 [M+H]⁺.

Step F: (R)-6-(3-(3-(3-Hydroxy-1-methyl-2-oxopyrrolidin-3-yl)-1-methyl-1H-pyrazol-5-yl)phenyl)picolinamide. The (R) and (S) enantiomers of (R,S)-6-(3-(3-(3-hydroxy-1-methyl-2-oxopyrrolidin-3-yl)-1-methyl-1H-pyrazol-5-yl)phenyl)picolinamide (300 mg, 0.77 mmol) was further purified by SFC using an SFC column, such as a DAICEL CHIRALPAK® IC column (250 mm×30 mm×5 μm (isocratic elution: EtOH (containing 0.1% of 25% aqueous NH₃ solution): supercritical CO₂, 55%: 45%). The second eluting product was (R)-6-(3-(3-(3-Hydroxy-1-methyl-2-oxopyrrolidin-3-yl)-1-methyl-1H-pyrazol-5-yl)phenyl)picolinamide, which was further purified by preparative HPLC using an HPLC column, such as a Phenomenex Gemini NX—C18 75 mm×30 mm×3 μm column (eluent: 15% to 45%,CH₃CN and H₂O (with 0.05% NH₄OH+10 mM NH₄HCO₃)) to afford the product. The product was suspended in water (10 mL), the mixture was frozen by insertion into a −78° C. bath, and then lyophilized to dryness to afford (R)-6-(3-(3-(3-Hydroxy-1-methyl-2-oxopyrrolidin-3-yl)-1-methyl-1H-pyrazol-5-yl)phenyl)picolinamide (86.5 mg, 29%) as a white solid. LCMS (ESI): Mass calcd. For C₂₁H₂₁N₅O₃ 391.2 m/z found 392.2 [M+H]⁺. ¹H NMR (400 MHz, DMSO-d₆) δ 8.45-8.39 (m, 2H), 8.35 (d, J=7.7 Hz, 1H), 8.27 (d, J=7.7 Hz, 1H), 8.11-8.06 (m, 1H), 8.04-8.00 (m, 1H), 7.71 (s, 1H), 7.67-7.62 (m, 1H), 7.61-7.56 (m, 1H), 6.57 (s, 1H), 5.89 (s, 1H), 3.86 (s, 3H), 3.42-3.38 (m, 2H), 2.78 (s, 3H), 2.73-2.67 (m, 1H), 2.20-2.09 (m, 1H).

### Example 31: (R)-6-(3-(3-(3-Hydroxy-1-methyl-2-oxopyrrolidin-3-yl)-1H-pyrazol-5-yl)phenyl)picolinamide

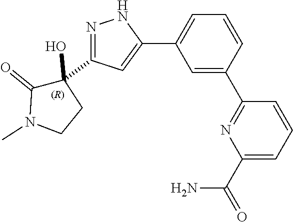

Step A: 3,5-Dibromo-1-((2-(trimethylsilyl)ethoxy)methyl)-1H-pyrazole. 2-(Trimethylsilyl)ethoxymethyl chloride (6.66 mL, 37.6 mmol) was added to a solution of 3,5-dibromo-1H-pyrazole (5.00 g, 22.1 mmol), Et₃N (4.62 mL, 33.2 mmol), and methylene chloride (60 mL). This mixture was stirred at room temperature for 30 minutes and then concentrated to dryness under reduced pressure. The product was purified by FCC (eluent: petroleum ether:ethyl acetate=1:0 to 3:1) to yield 3,5-dibromo-1-((2-(trimethylsilyl)ethoxy)methyl)-1H-pyrazole (4.7 g, 46%) as a colorless oil. ¹H NMR (400 MHz, CDCl₃) δ 6.40-6.32 (m, 1H), 5.45-5.41 (m, 2H), 3.65-3.59 (m, 2H), 0.93-0.87 (m, 2H), −0.02 (s, 9H).

Step B: (R,S)-3-(3-Bromo-1-((2-(trimethylsilyl)ethoxy)methyl)-1H-pyrazol-5-yl)-3-hydroxy-1-methylpyrrolidin-2-one. n-Butyllithium (4.15 mL, 10.4 mmol, 2.5 M in hexanes) was added to a solution of 3,5-dibromo-1-((2-(trimethylsilyl)ethoxy)methyl)-1H-pyrazole (4.20 g, 9.40 mmol) in THF (10 mL) that had been cooled to −72° C. The resultant mixture was stirred at −72° C. for 30 minutes, treated with a solution of 1-methylpyrrolidine-2,3-dione (1.28 g, 11.3 mmol) in THF (20 mL), and then stirred at −72° C. for 1 h. The reaction mixture was then quenched with H₂O (50 mL) and extracted with DCM/MeOH (10:1, 50 mL×3). The combined organic extracts were washed with brine, dried over anhydrous Na₂SO₄, filtered, and concentrated to dryness under reduced pressure. The resulting residue was purified by FCC (eluent: petroleum ether:ethyl acetate=1:0 to 1:1) to yield (R,S)-3-(3-bromo-1-((2-(trimethylsilyl)ethoxy)methyl)-1H-pyrazol-5-yl)-3-hydroxy-1-methylpyrrolidin-2-one (1.1 g, 27%) as a white solid. ¹H NMR (400 MHz, DMSO-d₆) δ 6.52-6.45 (m, 1H), 6.34-6.26 (m, 1H), 5.95-5.85 (m, 1H), 5.31-5.21 (m, 1H), 3.69-3.55 (m, 2H), 3.41-3.36 (m, 2H), 3.27-3.19 (m, 1H), 2.79 (s, 3H), 2.28-2.17 (m, 1H), 0.94-0.73 (m, 2H), 0.03 (s, 9H).

Step C: (R,S)-6-(3-(5-(3-Hydroxy-1-methyl-2-oxopyrrolidin-3-yl)-1-((2-(trimethylsilyl)ethoxy)methyl)-1H-pyrazol-3-yl)phenyl)picolinamide. (R,S)-3-(3-Bromo-1-((2-(trimethylsilyl)ethoxy)methyl)-1H-pyrazol-5-yl)-3-hydroxy-1-methylpyrrolidin-2-one (400 mg, 1.02 mmol), (3-(6-carbamoylpyridin-2-yl)phenyl)boronic acid (248 mg, 1.02 mmol), and K₃PO₄ (653 mg, 3.07 mmol) were added to a microwave tube and the resultant mixture dissolved in 1,4-dioxane (1.6 mL) and H₂O (0.4 mL). This mixture was purged with Ar for 5 minutes and then treated with Pd(dtbpf)Cl₂ (67 mg, 0.10 mmol). The resultant mixture was purged with Ar for another 5 minutes and then subjected to microwave irradiation at 80° C. for 1 hour. After this time, the mixture was poured into H₂O (10 mL), and extracted with EtOAc (10 mL×2). The combined organic extracts were washed with brine (10 mL), dried over anhydrous Na₂SO₄, filtered, and concentrated to dryness under reduced pressure. The product was purified by FCC (eluent: petroleum ether:ethyl acetate=1:0 to 0:1) to afford (R,S)-6-(3-(5-(3-hydroxy-1-methyl-2-oxopyrrolidin-3-yl)-1-((2-(trimethylsilyl)ethoxy)methyl)-1H-pyrazol-3-yl)phenyl)picolinamide (300 mg, 52%) as a yellow solid. MS (ESI): Mass calcd. for C₂₆H₃₃N₅O₄Si, 507.2; m/z found 508.3 [M+H]⁺.

Step D: (R,S)-6-(3-(5-(3-Hydroxy-1-methyl-2-oxopyrrolidin-3-yl)-1H-pyrazol-3-yl)phenyl)picolinamide. (R,S)-6-(3-(5-(3-Hydroxy-1-methyl-2-oxopyrrolidin-3-yl)-1-((2-(trimethylsilyl)ethoxy)methyl)-1H-pyrazol-3-yl)phenyl)picolinamide (270 mg, 0.532 mmol) was added to a solution of TBAF (1.60 mL, 1.0 M in THF, 1.60 mmol). The resultant mixture was heated at 90° C. for 16 h. The mixture was concentrated to dryness under reduced pressure and the product was purified by FCC (eluent: petroleum ether:ethyl acetate=1:0 to 0:1) to yield (R,S)-6-(3-(5-(3-hydroxy-1-methyl-2-oxopyrrolidin-3-yl)-1-((2-(trimethylsilyl)ethoxy)methyl)-1H-pyrazol-3-yl)phenyl)picolinamide (130 mg, 60%) as a yellow solid. MS (ESI): Mass calcd. for C₂₀H₁₉N₅O₃, 377.4; m/z found 378.2 [M+H]⁺.

Step E: (R)-6-(3-(3-(3-Hydroxy-1-methyl-2-oxopyrrolidin-3-yl)-1H-pyrazol-5-yl)phenyl)picolinamide. The (R) and (S) enantiomers of (R,S)-6-(3-(5-(3-Hydroxy-1-methyl-2-oxopyrrolidin-3-yl)-1H-pyrazol-3-yl)phenyl)picolinamide (130 mg, 0.344 mmol) were separated by SFC using an SFC column, such as a DAICEL CHIRALCEL® OD-H 250 mm×30 mm, 5 μm column (isocratic elution of EtOH:supercritical CO₂, 45%:55%). The second eluting product was (R)-6-(3-(3-(3-hydroxy-1-methyl-2-oxopyrrolidin-3-yl)-1H-pyrazol-5-yl)phenyl)picolinamide. The fractions containing (R)-6-(3-(3-(3-hydroxy-1-methyl-2-oxopyrrolidin-3-yl)-1H-pyrazol-5-yl)phenyl)picolinamide were collected and concentrated under reduced pressure. This product was then suspended in water (10 mL), the mixture was frozen by insertion into a −78° C. bath, and then lyophilized to dryness to afford (R)-6-(3-(3-(3-Hydroxy-1-methyl-2-oxopyrrolidin-3-yl)-1H-pyrazol-5-yl)phenyl)picolinamide (32.6 mg, 25.0%) as a colorless solid. MS (ESI): Mass calcd. for C₂₀H₁₉N₅O₃, 377.2; m/z found, 378.0 [M+H]⁺. ¹H NMR (400 MHz, DMSO-d₆) δ 13.41-12.72 (m, 1H), 8.66-8.50 (m, 1H), 8.47-8.33 (m, 1H), 8.30-8.21 (m, 2H), 8.13-8.07 (m, 1H), 8.05-8.00 (m, 1H), 7.90-7.75 (m, 2H), 7.62-7.51 (m, 1H), 6.87 (s, 1H), 6.40-5.79 (m, 1H), 3.41-3.41 (m, 2H), 2.79 (s, 3H), 2.53-2.53 (m, 1H), 2.30-2.08 (m, 1H).

### Example 32: (R)-3-(5-(3-(4-Aminopyrido[3,2-d]pyrimidin-6-yl-2-d)phenyl)thiazol-2-yl)-3-hydroxy-1-methylpyrrolidin-2-one

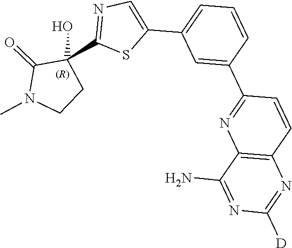

Step A: (R,S)-3-(5-(3-(4-Aminopyrido[3,2-d]pyrimidin-6-yl-2-d)phenyl)thiazol-2-yl)-3-hydroxy-1-methylpyrrolidin-2-one. (R,S)-3-Hydroxy-1-methyl-3-(5-(3-(4,4,5,5-tetramethyl-1,3,2-dioxaborolan-2-yl)phenyl)thiazol-2-yl)pyrrolidin-2-one (obtained from Example 28, Step C, 0.4 g, 0.9 mmol), 6-chloropyrido[3,2-d]pyrimidin-2-d-4-amine (Intermediate 17, 0.26 g, 1.5 mmol), and K₃PO₄ (0.62 g, 2.9 mmol) were added to a microwave tube and the resultant mixture was dissolved in 1,4-dioxane (2.4 mL) and H₂O (0.6 mL). This mixture was purged with N₂ for 5 minutes and then treated with Pd(dtbpf)Cl₂ (63 mg, 0.1 mmol). The resultant mixture was purged with N₂ for another 5 minutes and then subjected to microwave irradiation for 1 hour at 100° C. before cooling to room temperature. The mixture was then concentrated to dryness under reduced pressure. The product was purified by preparative HPLC using an HPLC column, such as a Phenomenex Gemini-NX, 150×30 mm×5 m column (eluent: 20% to 50%, CH₃CN-water (with 0.04% NH₄OH+10 mM NH₄HCO₃)). The product was then suspended in water (10 mL), the mixture was frozen by insertion into a −72° C. bath, and then lyophilized to dryness to afford (R,S)-3-(5-(3-(4-aminopyrido[3,2-d]pyrimidin-6-yl-2-d)phenyl)thiazol-2-yl)-3-hydroxy-1-methylpyrrolidin-2-one (100 mg, 24%) as a colorless solid. MS (ESI): Mass calcd. for C₂₁H₁₇DN₆O₂S, 419.1; m/z found, 420.1 [M+H]⁺.

Step B: (R)-3-(5-(3-(4-Aminopyrido[3,2-d]pyrimidin-6-yl-2-d)phenyl)thiazol-2-yl)-3-hydroxy-1-methylpyrrolidin-2-one. The (R) and (S) enantiomers of (R,S)-3-(5-(3-(4-aminopyrido[3,2-d]pyrimidin-6-yl-2-d)phenyl)thiazol-2-yl)-3-hydroxy-1-methylpyrrolidin-2-one (100 mg, 0.238 mmol) were separated by SFC using an SFC column, such as a DAICEL CHIRALCEL® OD-H 250×30 mm×5 m column (isocratic elution of EtOH:supercritical CO₂, 45%:55%). The second eluting product was (R)-3-(5-(3-(4-aminopyrido[3,2-d]pyrimidin-6-yl-2-d)phenyl)thiazol-2-yl)-3-hydroxy-1-methylpyrrolidin-2-one. The fractions of (R)-3-(5-(3-(4-aminopyrido[3,2-d]pyrimidin-6-yl-2-d)phenyl)thiazol-2-yl)-3-hydroxy-1-methylpyrrolidin-2-one were collected and concentrated under reduced pressure. This product was suspended in water (10 mL), the mixture was frozen by insertion into a −78° C. bath and then lyophilized to dryness to afford (R)-3-(5-(3-(4-aminopyrido[3,2-d]pyrimidin-6-yl-2-d)phenyl)thiazol-2-yl)-3-hydroxy-1-methylpyrrolidin-2-one (38.1 mg, 38%) as a colorless solid. MS (ESI): Mass calcd. for C₂₁H₁₇DN₆O₂S, 419.1; m/z found, 420.0 [M+H]⁺. ¹H NMR (400 MHz, DMSO-d₆) δ 8.66-8.63 (m, 1H), 8.56 (d, J=9.0 Hz, 1H), 8.41-8.37 (m, 1H), 8.34 (s, 1H), 8.25 (br s, 1H), 8.17 (d, J=8.8 Hz, 1H), 8.03 (br s, 1H), 7.76-7.71 (m, 1H), 7.64-7.58 (m, 1H), 6.96 (s, 1H), 3.59-3.43 (m, 2H), 2.83 (s, 3H), 2.78-2.71 (m, 1H), 2.32-2.24 (m, 1H).

### Example 33: (R)-3-(2-(3-(4-Amino-2-d-pyrido[3,2-d]pyrimidin-6-yl)phenyl)thiazol-4-yl)-3-hydroxy-1-methylpyrrolidin-2-one

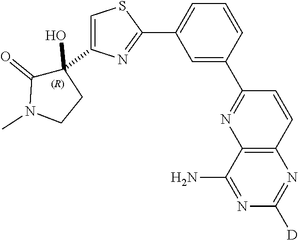

Step A: (R,S)-3-(1-Ethoxyvinyl)-3-hydroxy-1-methylpyrrolidin-2-one. tert-Butyllithium (10.2 mL, 13.3 mmol, 1.3 M in pentane) was added dropwise to a solution of ethoxyethene (3.00 mL, 31.0 mmol) and anhydrous THF (40 mL) that had been cooled to −72° C. The resultant mixture was warmed to 0° C., and stirred until the color of the solution turned pale yellow. The mixture was then cooled to −72° C., treated with a solution of 1-methylpyrrolidine-2,3-dione (500 mg) and anhydrous THF (10 mL), and stirred at −72° C. for 1 h. The mixture was then treated with saturated aqueous NaHCO₃ solution (5 mL) and water (15 mL), and extracted with ethyl acetate (30 mL×3). The combined organic extracts were dried over anhydrous Na₂SO₄, filtered, and concentrated to dryness under reduced pressure. The product was purified by preparative HPLC using an HPLC column, such as a C18 column (eluent: 5% to 50%, CH₃CN and H₂O) to afford (R,S)-3-(1-ethoxyvinyl)-3-hydroxy-1-methylpyrrolidin-2-one (160 mg, 17%) as a colorless oil. MS (ESI): Mass calcd. for C₉H₁₅NO₃, 185.1, m/z found, 186.1 [M+H]⁺.

Step B: (R,S)-3-(2-Bromoacetyl)-3-hydroxy-1-methylpyrrolidin-2-one. N-Bromosuccinimide (778 mg, 4.37 mmol) was added in portions to a solution of (R,S)-3-(1-ethoxyvinyl)-3-hydroxy-1-methylpyrrolidin-2-one (810 mg, 4.37 mmol) and acetone/water (9:1, 12 mL) that had been cooled to 0° C. The resultant mixture was stirred at room temperature for 1 hour. The mixture was then partitioned between ethyl acetate (30 mL) and water (30 mL). The organic layers were washed with brine (20 mL), dried over anhydrous Na₂SO₄, filtered, and concentrated to dryness under reduced pressure to give (R,S)-3-(2-bromoacetyl)-3-hydroxy-1-methylpyrrolidin-2-one (1.19 g) as a colorless oil which was used for the next reaction without further purification. MS (ESI): Mass calcd. for C₇H₁₀BrNO₃ 235.0 m/z found, 236.0 [M+H]⁺.

Step C: (R,S)-3-(2-(3-Chlorophenyl)thiazol-4-yl)-3-hydroxy-1-methylpyrrolidin-2-one. (R,S)-3-(2-Bromoacetyl)-3-hydroxy-1-methylpyrrolidin-2-one (550 mg, 2.33 mmol), 3-chlorobenzothioamide (400 mg, 2.33 mmol), and ethanol (10 mL) were added to a microwave tube. The resultant mixture was subjected to microwave irradiation at 100° C. for 1 h. After this time, the mixture was cooled to room temperature and concentrated to dryness under reduced pressure. The resulting residue was purified by FCC (eluent: petroleum ether:ethyl acetate=10:1 to 0:1) to give (R,S)-3-(2-(3-chlorophenyl)thiazol-4-yl)-3-hydroxy-1-methylpyrrolidin-2-one (350 mg, 48%) as a white solid. MS (ESI): Mass calcd. for C₁₄H₁₃ClN₂O₂S, 308.0; m/z found, 309.2 [M+H]⁺.

Step D: (R,S)-3-Hydroxy-1-methyl-3-(2-(3-(4,4,5,5-tetramethyl-1,3,2-dioxaborolan-2-yl)phenyl)thiazol-4-yl)pyrrolidin-2-one. (R,S)-3-(2-(3-Chlorophenyl)thiazol-4-yl)-3-hydroxy-1-methylpyrrolidin-2-one (350 mg, 1.13 mmol), 4,4,4′,4′,5,5,5′,5′-octamethyl-2,2′-bi(1,3,2-dioxaborolane) (576 mg, 2.27 mmol), and KOAc (334 mg, 3.40 mmol) were added to a round-bottomed flask and the resultant mixture dissolved in 1,4-dioxane (30 mL). The mixture was sparged with Ar for 5 minutes and then treated with chloro(2-dicyclohexylphosphino-2,4,6-triisopropyl-1,1-biphenyl)[2-(2-amino-1,1-biphenyl)]palladium(II) (89.2 mg, 0.113 mmol). The mixture was sparged with Ar for another 5 minutes and then heated at 110° C. for 16 h under Ar atmosphere. After this time, the mixture was cooled to room temperature, filtered through a pad of diatomaceous earth and the pad washed with ethyl acetate (20 mL×2). The filtrate was concentrated to dryness under reduced pressure to afford (R,S)-3-hydroxy-1-methyl-3-(2-(3-(4,4,5,5-tetramethyl-1,3,2-dioxaborolan-2-yl)phenyl)thiazol-4-yl)pyrrolidin-2-one (850 mg) as a black solid, which was used in the next step without further purification. MS (ESI): Mass calcd. for C₂₀H₂₅BN₂O₄S, 400.2; m/z found, 401.1 [M+1]⁺.

Step E: (R,S)-3-(2-(3-(4-Amino-2-d-pyrido[3,2-d]pyrimidin-6-yl)phenyl)thiazol-4-yl)-3-hydroxy-1-methylpyrrolidin-2-one. (R,S)-3-Hydroxy-1-methyl-3-(2-(3-(4,4,5,5-tetramethyl-1,3,2-dioxaborolan-2-yl)phenyl)thiazol-4-yl)pyrrolidin-2-one (0.32 mmol), 6-chloropyrido[3,2-d]pyrimidin-2-d-4-amine (Intermediate 17, 70 mg, 0.4 mmol), and K₃PO₄ (245 mg, 1.15 mmol) were added to a microwave tube, and the resultant mixture dissolved in 1,4-dioxane/H₂O (4:1, 2.5 mL). This mixture was sparged with Ar for 5 minutes and then treated with Pd(dtbpf)Cl₂ (25 mg, 0.039 mmol). The reaction mixture was sparged with Ar for another 5 minutes and then subjected to microwave irradiation at 100° C. for 1 h. The reaction was then cooled to room temperature, poured into H₂O (20 mL), and extracted with ethyl acetate (30 mL×4). The combined organic extracts were washed with brine (20 mL), dried over anhydrous Na₂SO₄, filtered, and concentrated to dryness under reduced pressure. The resulting residue was purified by FCC (eluent: petroleum ether:ethyl acetate=1:0 to 0:1, then ethyl acetate:methanol=10:1) to afford (R,S)-3-(2-(3-(4-amino-2-d-pyrido[3,2-d]pyrimidin-6-yl)phenyl)thiazol-4-yl)-3-hydroxy-1-methylpyrrolidin-2-one (100 mg, 56%) as a white solid. MS (ESI): Mass calcd. for C₂₁H₁₇DN₆O₂S, 419.1; m/z found, 420.1 [M+1]⁺.

Step F: (R)-3-(2-(3-(4-Amino-2-d-pyrido[3,2-d]pyrimidin-6-yl)phenyl)thiazol-4-yl)-3-hydroxy-1-methylpyrrolidin-2-one. The (R) and (S) enantiomers of (R,S)-3-(2-(3-(4-amino-2-d-pyrido[3,2-d]pyrimidin-6-yl)phenyl)thiazol-4-yl)-3-hydroxy-1-methylpyrrolidin-2-one (100 mg) were separated by SFC using an SFC column, such as a DAICEL CHIRALPAK® OJ-H 250 mm×30 mm×5 m column (isocratic elution EtOH:supercritical CO₂, 30%:70%). The second eluting product, (R)-3-(2-(3-(4-amino-2-d-pyrido[3,2-d]pyrimidin-6-yl)phenyl)thiazol-4-yl)-3-hydroxy-1-methylpyrrolidin-2-one, was collected and concentrated under reduced pressure. This product was suspended in water (10 mL), the mixture was frozen by insertion into a −78° C. bath, and then lyophilized to dryness to afford (R)-3-(2-(3-(4-amino-2-d-pyrido[3,2-d]pyrimidin-6-yl)phenyl)thiazol-4-yl)-3-hydroxy-1-methylpyrrolidin-2-one (18 mg, 18%) as a white solid. MS (ESI): Mass calcd. for C₂₁H₁₇DN₆O₂S, 419.1; m/z found, 420.1 [M+1]⁺. ¹H NMR (400 MHz, DMSO-d₆) δ 8.71-8.67 (m, 1H), 8.57-8.53 (m, 1H), 8.51 (d, J=8.8 Hz, 1H), 8.19 (d, J=9.0 Hz, 1H), 8.15 (br s, 1H), 8.09-8.02 (m, 2H), 7.71-7.63 (m, 2H), 6.28 (br s, 1H), 3.63-3.55 (m, 1H), 3.52-3.44 (m, 1H), 2.85 (s, 3H), 2.73-2.64 (m, 1H), 2.27-2.18 (m, 1H).

### Example 34: (R)-3-(3-(3-(4-Aminopyrido[3,2-d]pyrimidin-6-yl-2-d)phenyl)-1H-1,2,4-triazol-5-yl)-3-hydroxy-1-methylpyrrolidin-2-one

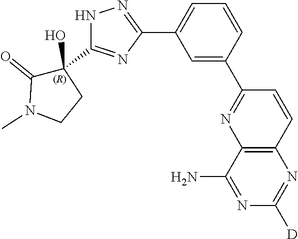

Step A: 3,5-Dibromo-1-((2-(trimethylsilyl)ethoxy)methyl)-1H-1,2,4-triazole. 2-(Trimethylsilyl)ethoxymethyl chloride (2.7 mL, 15 mmol) was added dropwise to a mixture of 3,5-dibromo-1H-1,2,4-triazole (3.5 g, 15 mmol), Et₃N (3.0 mL, 22 mmol), molecular sieves (3.5 g), and methylene chloride (45 mL). The resultant mixture was stirred at room temperature for 30 minutes. After this time, the mixture was concentrated to dryness under reduced pressure. The resulting residue was purified by FCC (eluent: petroleum ether:EtOAc=0:1 to 5:1) to afford 3,5-dibromo-1-((2-(trimethylsilyl)ethoxy)methyl)-1H-1,2,4-triazole (3.7 g, 59%) as a colorless solid.

Step B: (R,S)-3-(3-Bromo-1-((2-(trimethylsilyl)ethoxy)methyl)-1H-1,2,4-triazol-5-yl)-3-hydroxy-1-methylpyrrolidin-2-one. n-Butyllithium (3.2 mL, 2.5 M in hexanes, 8.0 mmol) was added to a solution of 3,5-dibromo-1-((2-(trimethylsilyl)ethoxy)methyl)-1H-1,2,4-triazole (2.38 g, 6.66 mmol) and dry THF (25 mL) that had been cooled to −72° C. The resultant mixture was stirred at −72° C. for 30 minutes before it was treated with 1-methylpyrrolidine-2,3-dione (905 mg, 8.00 mmol) by syringe. The resultant mixture was stirred at −72° C. for 2 h. H₂O (30 mL) was added and the aqueous layer was extracted with EtOAc/MeOH (10:1, 30 mL×5). The organic extracts were concentrated to dryness under reduced pressure. The resulting residue was purified by preparative HPLC using an HPLC column, such as an Xtimate C18, 150 mm×40 mm×10 m column (eluent: 30% to 60%, water (0.04% NH₄OH+10 mM NH₄HCO₃)—CH₃CN) to afford (R,S)-3-(3-bromo-1-((2-(trimethylsilyl)ethoxy)methyl)-1H-1,2,4-triazol-5-yl)-3-hydroxy-1-methylpyrrolidin-2-one. (1.65 g, 63%) as a yellow solid. MS (ESI): Mass calcd. for C₁₃H₂₃BrN₄O₃Si, 390.1; m/z found, 391.0 [M+1]⁺.

Step C: 6-(3-(4,4,5,5-Tetramethyl-1,3,2-dioxaborolan-2-yl)phenyl)pyrido[3,2-d]pyrimidin-2-d-4-amine. A mixture of 6-(3-chlorophenyl)pyrido[3,2-d]pyrimidin-2-d-4-amine (200 mg, 0.776 mmol), 4,4,4′,4′,5,5,5′,5′-octamethyl-2,2′-bi(1,3,2-dioxaborolane) (394 mg, 1.55 mmol), KOAc (229 mg, 2.33 mmol), and 1,4-dioxane (8 mL) was purged with Ar for 5 minutes. Chloro(2-dicyclohexylphosphino-2′,4′,6′-triisopropyl-1,1′-biphenyl)[2-(2′-amino-1,1′-biphenyl)]palladium(II) (61 mg, 0.078 mmol) was added. The resultant mixture was purged with Ar for another 5 minutes and then subjected to microwave irradiation at 100° C. for 1 h. After this time, the mixture was cooled to room temperature, and concentrated to dryness under reduced pressure to afford the product, which was purified by FCC (eluent: petroleum ether:EtOAc=1:0 to 0:1) to give 6-(3-(4,4,5,5-tetramethyl-1,3,2-dioxaborolan-2-yl)phenyl)pyrido[3,2-d]pyrimidin-2-d-4-amine (170 mg, 53%) as a yellow solid. MS (ESI): Mass calcd. for C₁₉H₂₀BDN₄O₂, 349.2; m/z found, 350.1 [M+H]⁺.

Step D: (R,S)-3-(3-(3-(4-Aminopyrido[3,2-d]pyrimidin-6-yl-2-d)phenyl)-1-((2-(trimethylsilyl)ethoxy)methyl)-1H-1,2,4-triazol-5-yl)-3-hydroxy-1-methylpyrrolidin-2-one. A mixture of 6-(3-(4,4,5,5-tetramethyl-1,3,2-dioxaborolan-2-yl)phenyl)pyrido[3,2-d]pyrimidin-2-d-4-amine (150 mg, 0.430 mmol), (R,S)-3-(3-bromo-1-((2-(trimethylsilyl)ethoxy)methyl)-1H-1,2,4-triazol-5-yl)-3-hydroxy-1-methylpyrrolidin-2-one (252 mg, 0.644 mmol), K₃PO₄ (274 mg, 1.29 mmol), 1,4-dioxane (4 mL), and H₂O (1 mL) was purged with Ar for 5 minutes. 1,1′-Bis(di-tert butylphosphino)ferrocene palladium dichloride (28 mg, 0.043 mmol) was added to the mixture. The resultant mixture was purged with Ar for another 5 minutes and the mixture was subjected to microwave irradiation at 80° C. for 1 h. After this time, the mixture was cooled to room temperature and concentrated to dryness under reduced pressure. The resulting residue was purified by FCC (eluent: petroleum ether:EtOAc=1:0 to 1:0) to afford (R,S)-3-(3-(3-(4-aminopyrido[3,2-d]pyrimidin-6-yl-2-d)phenyl)-1-((2-(trimethylsilyl)ethoxy)methyl)-1H-1,2,4-triazol-5-yl)-3-hydroxy-1-methylpyrrolidin-2-one (146 mg, 51%) as a brown solid. MS (ESI): Mass calcd. for C₂₆H₃₁DN₈O₃Si, 533.2; m/z found, 534.2 [M+H]⁺.

Step E: (R,S)-3-(3-(3-(4-Aminopyrido[3,2-d]pyrimidin-6-yl-2-d)phenyl)-1H-1,2,4-triazol-5-yl)-3-hydroxy-1-methylpyrrolidin-2-one. Tetrabutylammonium fluoride hydrate (0.65 mL, 1.0 M in THF, 0.65 mmol) was added to a flask containing (R,S)-3-(3-(3-(4-aminopyrido[3,2-d]pyrimidin-6-yl-2-d)phenyl)-1-((2-(trimethylsilyl)ethoxy)methyl)-1H-1,2,4-triazol-5-yl)-3-hydroxy-1-methylpyrrolidin-2-one (116 mg, 0.217 mmol). The resultant mixture was heated at 90° C. for 16 h. The mixture was concentrated to dryness under reduced pressure. The resulting residue was initially purified by FCC (eluent: petroleum ether:ethyl acetate=1:0 to 0:1), and then further purified by preparative HPLC using an HPLC column, such as a Boston Prime C18, 150×30 mm×5 μm column (eluent: 15% to 45%, water (0.05% NH₄OH+10 mM NH₄HCO₃)—CH₃CN) to afford (R,S)-3-(3-(3-(4-aminopyrido[3,2-d]pyrimidin-6-yl-2-d)phenyl)-1H-1,2,4-triazol-5-yl)-3-hydroxy-1-methylpyrrolidin-2-one (68.1 mg, 72%) as a white solid. MS (ESI): Mass calcd. for C₂₀H₁₇DN₈O₂, 403.2; m/z found, 404.2 [M+H]⁺.

Step F: (R)-3-(3-(3-(4-Amino-2-d-pyrido[3,2-d]pyrimidin-6-yl)phenyl)-1H-1,2,4-triazol-5-yl)-3-hydroxy-1-methylpyrrolidin-2-one. The (R) and (S) enantiomers of (R,S)-3-(3-(3-(4-aminopyrido[3,2-d]pyrimidin-6-yl-2-d)phenyl)-1H-1,2,4-triazol-5-yl)-3-hydroxy-1-methylpyrrolidin-2-one (68.1 mg) were separated by SFC using an SFC column, such as a DAICEL CHIRALPAK® AD, 250×30 mm×10 m column (isocratic elution IPA: supercritical CO₂, 55%:45%). The first eluting product, (R)-3-(3-(3-(4-amino-2-d-pyrido[3,2-d]pyrimidin-6-yl)phenyl)-1H-1,2,4-triazol-5-yl)-3-hydroxy-1-methylpyrrolidin-2-one, was collected and concentrated under reduced pressure. This product was suspended in water (10 mL), the mixture was frozen by insertion into a −78° C. bath, and then lyophilized to dryness to afford (R)-3-(3-(3-(4-amino-2-d-pyrido[3,2-d]pyrimidin-6-yl)phenyl)-1H-1,2,4-triazol-5-yl)-3-hydroxy-1-methylpyrrolidin-2-one (19 mg, 26%) as a white solid. MS (ESI): Mass calcd. for C₂₀H₁₇DN₈O₂, 403.2; m/z found, 404.1 [M+H]⁺. ¹H NMR (400 MHz, DMSO-d₆) δ 14.75-13.50 (m, 1H), 8.77 (s, 1H), 8.54-8.42 (m, 2H), 8.18 (d, J=8.8 Hz, 1H), 8.12-7.99 (m, 3H), 7.68-7.60 (m, 1H), 6.52 (br s, 1H), 3.60-3.52 (m, 1H), 3.52-3.45 (m, 1H), 2.87-2.71 (m, 4H), 2.31-2.15 (m, 1H).

### Example 35: (R)-3-(1-(3-(4-Aminopyrido[3,2-d]pyrimidin-6-yl-2-d)phenyl)-1H-1,2,3-triazol-4-yl)-3-hydroxy-1-methylpyrrolidin-2-one

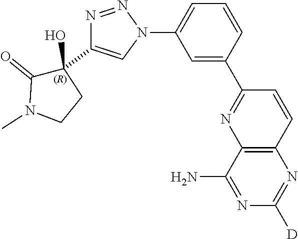

Step A: 1-Azido-3-bromobenzene. Azidotrimethylsilane (435 mg, 3.78 mmol) was added dropwise to a solution of 3-bromoaniline (500 mg, 2.91 mmol) and CH₃CN (10 mL) that had been cooled to 0° C. Then tert-butyl nitrite (360 mg, 3.49 mmol) was added. The mixture was stirred for 2 h with gradual warming to room temperature to yield 1-azido-3-bromobenzene. The reaction mixture was used in the next step without further purification.

Step B: (R)-3-(1-(3-Bromophenyl)-1H-1,2,3-triazol-4-yl)-3-hydroxy-1-methylpyrrolidin-2-one. (R)-3-Ethynyl-3-hydroxy-1-methylpyrrolidin-2-one (Intermediate 1, 184 mg, 1.32 mmol) was added to a solution of 1-azido-3-bromobenzene (574 mg, 2.90 mmol, in 10 mL CH₃CN), sodium 2-(1,2-dihydroxyethyl)-4-hydroxy-5-oxo-2,5-dihydrofuran-3-olate (52 mg, 0.26 mmol), copper(II) sulfate (42 mg, 0.26 mmol), DMF (10 mL), and H₂O (2 mL). The resultant mixture was heated at 100° C. for 16 h. The mixture was then cooled to room temperature, quenched with H₂O (50 mL), and extracted with ethyl acetate (50 mL×3). The combined organic extracts were washed with brine, dried over anhydrous Na₂SO₄, filtered, and concentrated to dryness under reduced pressure. The product was purified by FCC (eluent: petroleum ether:ethyl acetate=10:1 to 0:1) to afford (R)-3-(1-(3-bromophenyl)-1H-1,2,3-triazol-4-yl)-3-hydroxy-1-methylpyrrolidin-2-one (360 mg, 81%) as a yellow oil. MS (ESI): Mass calcd. for C₁₃H₁₃BrN₄O₂, 336.0; m/z found, 337.0 [M+1]⁺.

Step C: (R)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one. (R)-3-(1-(3-Bromophenyl)-1H-1,2,3-triazol-4-yl)-3-hydroxy-1-methylpyrrolidin-2-one (350 mg, 1.0 mmol), 4,4,4′,4′,5,5,5′,5′-octamethyl-2,2′-bi(1,3,2-dioxaborolane) (395 mg, 1.56 mmol), KOAc (306 mg, 3.12 mmol), and 1,4-dioxane (10 mL) were added to a microwave tube. The mixture was sparged with N₂ for 5 minutes and then treated with Pd(dppf)Cl₂ (76 mg, 0.10 mmol). The resultant mixture was subjected to microwave irradiation at 90° C. for 1 h. The mixture was then cooled to room temperature and concentrated to dryness under reduced pressure. The product was purified by FCC (eluent: petroleum ether:ethyl acetate=10:1 to 0:1) to give (R)-3-hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (280 mg, 61%) as a brown oil. MS (ESI): Mass calcd. for C₁₉H₂₅BN₄O₄, 384.2; m/z found, 385.2 [M+H]⁺.

Step D: (R)-3-(1-(3-(4-Aminopyrido[3,2-d]pyrimidin-6-yl-2-d)phenyl)-1H-1,2,3-triazol-4-yl)-3-hydroxy-1-methylpyrrolidin-2-one. 6-Chloropyrido[3,2-d]pyrimidin-2-d-4-amine (Intermediate 17, 90.0 mg, 0.50 mmol), (R)-3-hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (Intermediate 8, 194 mg, 0.51 mmol), K₃PO₄ (316 mg, 1.49 mmol), 1,4-dioxane (5 mL), and H₂O (1 mL) were added to a microwave tube. The mixture was sparged with Ar for 5 minutes and then treated with Pd(dtbpf)Cl₂ (32.0 mg, 0.05 mmol). The mixture was sparged with Ar for another 5 minutes and then the resultant mixture was subjected to microwave irradiation at 90° C. for 1 h. The mixture was cooled to room temperature and concentrated to dryness under reduced pressure. The product was initially purified by FCC (eluent: petroleum ether:ethyl acetate=10:1 to 0:1). The product was then further purified by preparative HPLC using an HPLC column, such as a Phenomenex Gemini-NX C18, 75×30 mm×3 m column (eluent: 20% to 50%, CH₃CN and H₂O (with 0.05% NH₄OH+10 mM NH₄HCO₃)). The product was suspended in water (10 mL), the mixture was frozen by insertion into a −78° C. bath, and then lyophilized to dryness to afford (R)-3-(1-(3-(4-aminopyrido[3,2-d]pyrimidin-6-yl-2-d)phenyl)-1H-1,2,3-triazol-4-yl)-3-hydroxy-1-methylpyrrolidin-2-one (74.1 mg, 36.0%) as a white solid. MS (ESI): Mass calcd. for C₂₀H₁₇DN₈O₂, 403.2; m/z found, 404.1 [M+H]⁺. ¹H NMR (400 MHz, DMSO-d₆) δ 8.99 (s, 1H), 8.84 (s, 1H), 8.62 (d, J=9.0 Hz, 1H), 8.53 (d, J=7.9 Hz, 1H), 8.36 (br s, 1H), 8.18 (d, J=8.8 Hz, 1H), 8.11-8.03 (m, 2H), 7.79-7.70 (m, 1H), 6.31 (s, 1H), 3.54-3.43 (m, 2H), 2.83 (s, 3H), 2.78-2.70 (m, 1H), 2.35-2.23 (m, 1H).

### Example 36: (S)-3-(1-(3-(4-Aminopyrido[3,2-d]pyrimidin-6-yl-2-d)phenyl)-1H-1,2,3-triazol-4-yl)-3-hydroxy-1-methylpyrrolidin-2-one

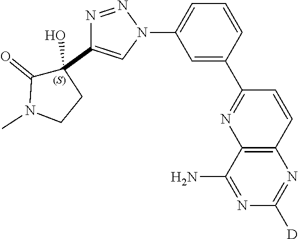

Step A: (S)-3-(1-(3-Bromophenyl)-1H-1,2,3-triazol-4-yl)-3-hydroxy-1-methylpyrrolidin-2-one. (S)-3-Ethynyl-3-hydroxy-1-methylpyrrolidin-2-one (Intermediate 2, 202 mg, 1.45 mmol) was added to a solution of 1-azido-3-bromobenzene (633 mg, 3.20 mmol in 10 mL CH₃CN), sodium 2-(1,2-dihydroxyethyl)-4-hydroxy-5-oxo-2,5-dihydrofuran-3-olate (57 mg, 0.29 mmol), copper(II) sulfate (46 mg, 0.29 mmol), DMF (15 mL), and H₂O (3 mL). The mixture was heated at 100° C. for 16 hours. The mixture was then cooled to room temperature, quenched with H₂O (80 mL), and extracted with ethyl acetate (60 mL×3). The combined organic extracts were washed with brine, dried over anhydrous Na₂SO₄, filtered, and concentrated to dryness. The resulting residue was purified by FCC (eluent: petroleum ether:ethyl acetate=10:1 to 0:1) to give (S)-3-(1-(3-bromophenyl)-1H-1,2,3-triazol-4-yl)-3-hydroxy-1-methylpyrrolidin-2-one (360 mg, 74%) as a yellow oil. MS (ESI): Mass calcd. or C₁₃H₁₃BrN₄O₂ 336.0 m/z found 337.0 [M+H]⁺.

Step B: (S)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one. (S)-3-(1-(3-Bromophenyl)-1H-1,2,3-triazol-4-yl)-3-hydroxy-1-methylpyrrolidin-2-one (360 mg, 1.1 mmol) was added to a mixture of 4,4,4′,4′,5,5,5′,5′-octamethyl-2,2′-bi(1,3,2-dioxaborolane (691 mg, 2.72 mmol), KOAc (315 mg, 3.21 mmol), and 1,4-dioxane (15 mL). The mixture was sparged with Ar for 5 minutes and then treated with Pd(dppf)Cl₂ (78 mg, 0.11 mmol). The mixture was sparged with Ar for another 5 minutes and then subjected to microwave irradiation at 85° C. for 1 hour. The mixture was then cooled to room temperature and concentrated to dryness. The product was purified by FCC (eluent: petroleum ether:ethyl acetate=1:0 to 0:1) to afford (S)-3-hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (350 mg, 53%) as a brown oil. MS (ESI): Mass calcd. for C₁₉H₂₅BN₄O₄, 384.2; m/z found, 385.2 [M+H]⁺.

Step C: (S)-3-(1-(3-(4-Aminopyrido[3,2-d]pyrimidin-6-yl-2-d)phenyl)-1H-1,2,3-triazol-4-yl)-3-hydroxy-1-methylpyrrolidin-2-one. (S)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (165 mg, 0.429 mmol), 6-chloropyrido[3,2-d]pyrimidin-2-d-4-amine (Intermediate 17, 95 mg, 0.47 mmol), K₃PO₄ (273 mg, 1.29 mmol), 1,4-dioxane (12 mL), and H₂O (3 mL) were added to a microwave tube. The mixture was sparged with Ar for 5 minutes and then treated with Pd(dtbpf)Cl₂ (28 mg, 0.043 mmol). The mixture was sparged with Ar for another 5 minutes and then subjected to microwave irradiation at 90° C. for 1 h before cooling to room temperature. The reaction mixture was concentrated to dryness under reduced pressure. The resulting residue was initially purified by FCC (eluent: petroleum ether:ethyl acetate=1:0 to 0:1, then ethyl acetate: methanol=1:0 to 10:1). This product was further purified by preparative HPLC using an HPLC column, such as a Phenomenex Gemini NX-C18 75 mm×30 mm×3 m column (eluent: 15% to 45%, CH₃CN and H₂O (with 10 mM NH₄HCO₃)). The product was then suspended in water (10 mL), the mixture was frozen by insertion into a −78° C. bath, and then lyophilized to dryness to afford (S)-3-(1-(3-(4-aminopyrido[3,2-d]pyrimidin-6-yl-2-d)phenyl)-1H-1,2,3-triazol-4-yl)-3-hydroxy-1-methylpyrrolidin-2-one (31.5 mg, 18%) as a white solid. MS (ESI): Mass calcd. for C₂₀H₁₇DN₈O₂, 403.2; m/z found, 404.1 [M+H]⁺. ¹H NMR (400 MHz, DMSO-d₆) δ 8.99 (s, 1H), 8.85 (m, 1H), 8.62 (d, J=9.0 Hz, 1H), 8.54 (d, J=7.8 Hz, 1H), 8.36 (s, 1H), 8.19 (d, J=8.8 Hz, 1H), 8.12-8.03 (m, 2H), 7.80-7.71 (m, 1H), 6.31 (s, 1H), 3.56-3.45 (m, 2H), 2.84 (s, 3H), 2.80-2.71 (m, 1H), 2.35-2.24 (m, 1H).

### Example 37: (S)-3-(5-(3-(4-Aminopyrido[3,2-d]pyrimidin-6-yl-2-d)phenyl)-1H-pyrazol-3-yl)-3-hydroxy-1-methylpyrrolidin-2-one

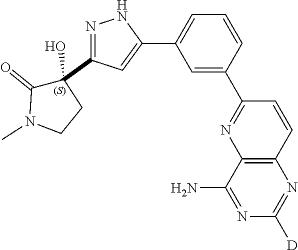

Step A: (R,S)-3-(3-(3-(4-aminopyrido[3,2-d]pyrimidin-6-yl-2-d)phenyl)-1-((2-(trimethylsilyl)ethoxy)methyl)-1H-pyrazol-5-yl)-3-hydroxy-1-methylpyrrolidin-2-one. 6-(3-(4,4,5,5-Tetramethyl-1,3,2-dioxaborolan-2-yl)phenyl)pyrido[3,2-d]pyrimidin-2-d-4-amine (Example 34, step C, 116 mg, 0.33 mmol), (R,S)-3-(3-bromo-1-((2-(trimethylsilyl)ethoxy)methyl)-1H-pyrazol-5-yl)-3-hydroxy-1-methylpyrrolidin-2-one (Example 31, step B, 194 mg, 0.49 mmol), and Cs₂CO₃ (325 mg, 0.99 mmol) were added to a microwave tube and the mixture dissolved in 1,4-dioxane (1.6 mL) and H₂O (0.4 mL). The resultant mixture was purged with Ar for 5 minutes and then treated with chloro(2-dicyclohexylphosphino-2,4,6-triisopropyl-1,1-biphenyl)[2-(2-amino-1,1-biphenyl)]palladium(II) (26 mg, 0.033 mmol). This mixture was purged with Ar for another 5 minutes and then subjected to microwave irradiation at 100° C. for 1 hour. The mixture was then concentrated to dryness under reduced pressure to give the product which was purified by FCC (eluent: petroleum ether:ethyl acetate=1:0 to 0:1) to afford (R,S)-3-(3-(3-(4-aminopyrido[3,2-d]pyrimidin-6-yl-2-d)phenyl)-1-((2-(trimethylsilyl)ethoxy)methyl)-1H-pyrazol-5-yl)-3-hydroxy-1-methylpyrrolidin-2-one (127 mg, 55%) as a yellow solid. MS (ESI): Mass calcd. for C₂₇H₃₂DN₇O₃Si, 532.3; m/z found, 533.5 [M+H]⁺.

Step B: (R,S)-3-(5-(3-(4-Aminopyrido[3,2-d]pyrimidin-6-yl-2-d)phenyl)-1H-pyrazol-3-yl)-3-hydroxy-1-methylpyrrolidin-2-one. (R,S)-3-(3-(3-(4-aminopyrido[3,2-d]pyrimidin-6-yl-2-d)phenyl)-1-((2-(trimethylsilyl)ethoxy)methyl)-1H-pyrazol-5-yl)-3-hydroxy-1-methylpyrrolidin-2-one (127 mg, 0.24 mmol) was added to a solution of TBAF (0.72 mL, 1.0 M in THF, 0.72 mmol). The resultant mixture was heated at 90° C. for 16 hours. The mixture was then concentrated to dryness under reduced pressure. The resulting residue was purified by FCC (eluent: ethyl acetate:methanol=1:0 to 10:1) to afford (R,S)-3-(5-(3-(4-aminopyrido[3,2-d]pyrimidin-6-yl-2-d)phenyl)-1H-pyrazol-3-yl)-3-hydroxy-1-methylpyrrolidin-2-one (71 mg, 65%) as a yellow solid. MS (ESI): Mass calcd. for C₂₁H₁₈DN₇O₂, 402.2; m/z found, 403.4 [M+H]⁺.

Step C: (S)-3-(5-(3-(4-Aminopyrido[3,2-d]pyrimidin-6-yl-2-d)phenyl)-1H-pyrazol-3-yl)-3-hydroxy-1-methylpyrrolidin-2-one. The (R) and (S) enantiomers of (R,S)-3-(3-(3-(4-amino-2-d-pyrido[3,2-d]pyrimidin-6-yl)phenyl)-1H-pyrazol-5-yl)-3-hydroxy-1-methylpyrrolidin-2-one (71 mg, 0.15 mmol) were separated by SFC using an SFC column, such as a DAICEL CHIRALCEL® OJ-H 250 mm×30 mm×5 m column (isocratic elution, EtOH:supercritical CO₂₌₅₀%:50%). The first eluting product was (S)-3-(5-(3-(4-aminopyrido[3,2-d]pyrimidin-6-yl-2-d)phenyl)-1H-pyrazol-3-yl)-3-hydroxy-1-methylpyrrolidin-2-one and the second eluting product was (R)-3-(5-(3-(4-aminopyrido[3,2-d]pyrimidin-6-yl-2-d)phenyl)-1H-pyrazol-3-yl)-3-hydroxy-1-methylpyrrolidin-2-one (Example 38). (S)-3-(5-(3-(4-Aminopyrido[3,2-d]pyrimidin-6-yl-2-d)phenyl)-1H-pyrazol-3-yl)-3-hydroxy-1-methylpyrrolidin-2-one was collected and concentrated under reduced pressure. This residue was then suspended in water (10 mL), the mixture was frozen by insertion into a −78° C. bath, and then lyophilized to dryness to afford (S)-3-(5-(3-(4-aminopyrido[3,2-d]pyrimidin-6-yl-2-d)phenyl)-1H-pyrazol-3-yl)-3-hydroxy-1-methylpyrrolidin-2-one (21 mg, 33%) as a colorless solid. MS (ESI): Mass calcd. for C₂₁H₁₈DN₇O₂, 402.4; m/z found, 403.1 [M+H]⁺. ¹H NMR (400 MHz, DMSO-d₆) δ 13.39-12.70 (m, 1H), 8.77-8.64 (m, 1H), 8.53 (d, J=8.8 Hz, 1H), 8.36 (d, J=7.9 Hz, 1H), 8.21-8.01 (m, 3H), 7.96-7.79 (m, 1H), 7.65-7.54 (m, 1H), 6.91 (s, 1H), 6.38-5.73 (m, 1H), 3.42-3.42 (m, 2H), 2.80 (s, 3H), 2.71-2.59 (m, 1H), 2.31-2.09 (m, 1H).

### Example 38: (R)-3-(5-(3-(4-Aminopyrido[3,2-d]pyrimidin-6-yl-2-d)phenyl)-1H-pyrazol-3-yl)-3-hydroxy-1-methylpyrrolidin-2-one

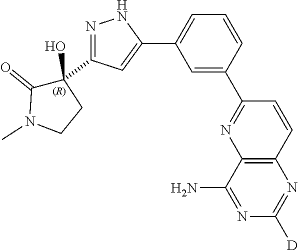

(R)-3-(5-(3-(4-Aminopyrido[3,2-d]pyrimidin-6-yl-2-d)phenyl)-1H-pyrazol-3-yl)-3-hydroxy-1-methylpyrrolidin-2-one, the second eluting product from Example 37, was further purified with preparative HPLC using an HPLC column, such as a Phenomenex Gemini NX-C18 75×30 mm×3 m column (eluent: 15% to 45%, CH₃CN and H₂O (with 0.05% NH₄OH and 10 mM NH₄HCO₃)). The product was suspended in water (10 mL), the mixture was frozen by insertion into a −72° C. bath, and then lyophilized to dryness to afford (R)-3-(5-(3-(4-aminopyrido[3,2-d]pyrimidin-6-yl-2-d)phenyl)-1H-pyrazol-3-yl)-3-hydroxy-1-methylpyrrolidin-2-one (12 mg, 17%) as a colorless solid. MS (ESI): Mass calcd. for C₂₁H₁₈DN₇O₂, 402.43; m/z found 403.1 [M+H]⁺. ¹H NMR (400 MHz, DMSO-d₆) δ 13.35-12.72 (m, 1H), 8.81-8.60 (m, 1H), 8.53 (d, J=8.8 Hz, 1H), 8.36 (d, J=7.9 Hz, 1H), 8.23-7.79 (m, 4H), 7.66-7.53 (m, 1H), 6.91 (s, 1H), 6.41-5.78 (m, 1H), 3.48-3.43 (m, 2H), 2.80 (s, 3H), 2.75-2.61 (m, 1H), 2.32-2.07 (m, 1H).

### Example 39: (R)-3-(2-(3-(4-Aminopyrido[3,2-d]pyrimidin-6-yl-2-d)phenyl)oxazol-5-yl)-3-hydroxy-1-methylpyrrolidin-2-one

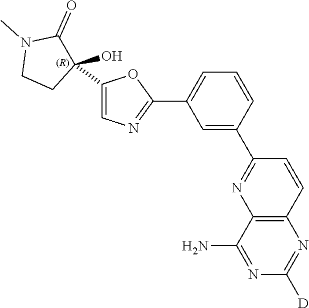

Step A: 2-(3-Chlorophenyl)oxazole. 1-Bromo-3-chlorobenzene (4.0 g, 21 mmol), oxazole (1.7 g, 25 mmol) and LiOtBu (3.3 g, 41 mmol) were added to 1,4-dioxane (40 mL). The mixture was sparged with N₂ for 2 minutes and then treated with Pd(PPh₃)₄ (2.4 g, 2.1 mmol). The mixture was sparged with N₂ for another 2 minutes and then heated at 100° C. for 12 hours under a N₂ atmosphere. After this time, the mixture was cooled to room temperature and filtered. The filtrate was diluted with water (80 mL) and extracted with ethyl acetate (80 mL×3). The combined organic extracts were washed with brine, dried over anhydrous Na₂SO₄, filtered, and concentrated under reduced pressure to give the product, which was purified by FCC (eluent: petroleum ether:ethyl acetate=1:0 to 7:3) to afford 2-(3-chlorophenyl)oxazole (3.5 g, 80%) as a colorless oil. MS (ESI): Mass calcd. for C₉H₆ClNO, 179.0; m/z found, 180.2 [M+H]⁺.

Step B: 5-Bromo-2-(3-chlorophenyl)oxazole. N-Bromosuccinimide (1.75 g, 9.83 mmol) was added to a mixture of 2-(3-chlorophenyl)oxazole (1.75 g, 9.74 mmol) and DMF (10 mL). The resultant mixture was subjected to microwave irradiation at 80° C. for 20 minutes. After this time, the mixture was diluted with water (80 mL) and extracted with ethyl acetate (80 mL×2). The combined organic extracts were washed with brine, dried over anhydrous Na₂SO₄, filtered, and concentrated under reduced pressure to give the product, which was purified by FCC (eluent: petroleum ether:ethyl acetate=1:0 to 20:1) to afford 5-bromo-2-(3-chlorophenyl)oxazole (3.0 g) as a white solid. 1H NMR (400 MHz, DMSO-d₆) δ 7.94-7.88 (m, 2H), 7.65-7.55 (m, 2H), 7.53 (s, 1H).

Step C: 4-Bromo-2-(3-chlorophenyl)oxazole. Lithium diisopropylamide (12 mL, 24 mmol, 2.0 M in THF) was added to a mixture of 5-bromo-2-(3-chlorophenyl)oxazole (3.0 g, 11.6 mmol) and THF (20 mL) that had been cooled to −72° C. The resultant mixture was stirred at −72° C. for 30 minutes. After this time, the mixture was warmed to room temperature, diluted with saturated aqueous NH₄Cl solution (60 mL), and extracted with ethyl acetate (60 mL×3). The combined organic extracts were washed with brine, dried over anhydrous Na₂SO₄, filtered, and concentrated under reduced pressure to give the product, which was purified by preparative HPLC using an HPLC column, such as a YMC-Triart Prep C18 150×40 mm×7 m column (eluent: 62% to 72%, H₂O (0.04% NH₄OH+10 mM NH₄HCO₃)—CH₃CN). This product was then suspended in water (10 mL), the mixture was frozen by insertion into a −78° C. bath, and then lyophilized to dryness to afford 4-bromo-2-(3-chlorophenyl)oxazole (700 mg, 23%) as a yellow solid. MS (ESI): Mass calcd. for C₉H₅BrClNO, 256.9 m/z; found 257.9 [M+H]⁺. ¹H NMR (400 MHz, DMSO-d₆) δ 8.54 (s, 1H), 7.96-7.91 (m, 2H), 7.67-7.56 (m, 2H).

Step D: (R,S)-3-(2-(3-Chlorophenyl)oxazol-5-yl)-3-hydroxy-1-methylpyrrolidin-2-one. n-Butyllithium (1.3 mL, 2.5 M in hexanes, 3.3 mmol) was added dropwise to a solution of 4-bromo-2-(3-chlorophenyl)oxazole (700 mg, 2.71 mmol) and THF (10 mL) that had been cooled to −72° C. The resultant mixture was stirred at −72° C. for 20 minutes before it was treated with a solution of 1-methylpyrrolidine-2,3-dione (371 mg, 3.28 mmol) in THF (5 mL). This mixture was stirred at −72° C. for 2 h. After this time, the mixture was diluted with saturated aqueous NH₄Cl solution (30 mL) and extracted with ethyl acetate (30 mL×3). The combined organic extracts were washed with brine, dried over anhydrous Na₂SO₄, filtered, and concentrated under reduced pressure to give the product, which was purified by FCC (eluent: petroleum ether:ethyl acetate=1:0 to 0:1) to afford (R,S)-3-(2-(3-chlorophenyl)oxazol-5-yl)-3-hydroxy-1-methylpyrrolidin-2-one (200 mg, 22%) as a colorless oil. MS (ESI): Mass calcd. for C₁₄H₁₃ClN₂O₃, 292.1; m/z found, 293.2 [M+H]⁺. ¹H NMR (400 MHz, DMSO-d₆) δ 7.97-7.88 (m, 2H), 7.66-7.53 (m, 2H), 7.28 (s, 1H), 6.53 (s, 1H), 3.50-3.39 (m, 2H), 2.82 (s, 3H), 2.65-2.55 (m, 1H), 2.29-2.18 (m, 1H).

Step E: (R,S)-3-Hydroxy-1-methyl-3-(2-(3-(4,4,5,5-tetramethyl-1,3,2-dioxaborolan-2-yl)phenyl)oxazol-5-yl)pyrrolidin-2-one. A mixture of (R,S)-3-(2-(3-chlorophenyl)oxazol-5-yl)-3-hydroxy-1-methylpyrrolidin-2-one (200 mg, 0.68 mmol), 4,4,4′,4′,5,5,5′,5′-octamethyl-2,2′-bi(1,3,2-dioxaborolane (696 mg, 2.74 mmol), KOAc (404 mg, 4.12 mmol), and 1,4-dioxane (10 mL) was sparged with N₂ for 2 minutes and then treated with chloro(2-dicyclohexylphosphino-2′,4′,6′-triisopropyl-1,1′-biphenyl)[2-(2′-amino-1,1′-biphenyl)]palladium(II) (160 mg, 0.20 mmol). The resultant mixture was sparged with N₂ for another 2 minutes and then subjected to microwave irradiation at 90° C. for 1 h. After this time, the mixture was cooled to room temperature and filtered. The filtrate was diluted with water (30 mL) and extracted with ethyl acetate (30 mL×3). The combined organic extracts were washed with brine, dried over anhydrous Na₂SO₄, filtered, and concentrated under reduced pressure to give the product, which was purified by FCC (eluent: petroleum ether:ethyl acetate=1:0 to 0:1) to afford (R,S)-3-hydroxy-1-methyl-3-(2-(3-(4,4,5,5-tetramethyl-1,3,2-dioxaborolan-2-yl)phenyl)oxazol-5-yl)pyrrolidin-2-one (270 mg, 76%) as a colorless oil. MS (ESI): Mass calcd. for C₂₀H₂₅BN₂O₅, 384.2; m/z found, 385.4 [M+H]⁺.

Step F: (R,S)-3-(2-(3-(4-Aminopyrido[3,2-d]pyrimidin-6-yl-2-d)phenyl)oxazol-5-yl)-3-hydroxy-1-methylpyrrolidin-2-one. A mixture of (R,S)-3-hydroxy-1-methyl-3-(2-(3-(4,4,5,5-tetramethyl-1,3,2-dioxaborolan-2-yl)phenyl)oxazol-5-yl)pyrrolidin-2-one (220 mg, 0.57 mmol), 6-chloropyrido[3,2-d]pyrimidin-2-d-4-amine (Intermediate 17, 136 mg, 0.75 mmol), K₃PO₄ (233 mg, 1.10 mmol), and 1,4-dioxane:H₂O (4:1, 10 mL) was sparged with N₂ for 2 minutes and then treated with Pd(dtbpf)Cl₂ (75.0 mg, 0.12 mmol).

The resultant mixture was sparged with N₂ for another 2 minutes and then subjected to microwave irradiation at 100° C. for 1 h. After this time, the mixture was cooled to room temperature and filtered. The filtrate was diluted with H₂O (20 mL) and extracted with ethyl acetate (20 mL×2). The combined organic extracts were washed with brine, dried over anhydrous Na₂SO₄, filtered, and concentrated under reduced pressure. The resulting residue was purified by preparative HPLC using an HPLC column, such as a Phenomenex Gemini-NX 80×40 mm×3 m column (eluent: 16% to 41%, H₂O (0.04% NH₄OH+10 mM NH₄HCO₃)—CH₃CN) to afford the product. This product was then suspended in water (10 mL), the mixture was frozen by insertion into a −78° C. bath, and then lyophilized to dryness to afford (R,S)-3-(2-(3-(4-aminopyrido[3,2-d]pyrimidin-6-yl-2-d)phenyl)oxazol-5-yl)-3-hydroxy-1-methylpyrrolidin-2-one (53 mg, 21%) as a yellow solid. MS (ESI): Mass calcd. for C₉H₅BrClNO, 403.4; m/z found 404.4 [M+H]⁺.

Step G: (R)-3-(2-(3-(4-Aminopyrido[3,2-d]pyrimidin-6-yl-2-d)phenyl)oxazol-5-yl)-3-hydroxy-1-methylpyrrolidin-2-one. The (R) and (S) enantiomers of (R,S)-3-(2-(3-(4-aminopyrido[3,2-d]pyrimidin-6-yl-2-d)phenyl)oxazol-5-yl)-3-hydroxy-1-methylpyrrolidin-2-one (53 mg, 0.13 mmol) were separated by SFC using an SFC column, such as a DAICEL CHIRALPAK® AD 250 mm×30 mm×10 m column (isocratic elution: EtOH (containing 0.1% of 25% aqueous NH₃ solution): supercritical CO₂, 55%:45%). The first eluting product was (R)-3-(2-(3-(4-aminopyrido[3,2-d]pyrimidin-6-yl-2-d)phenyl)oxazol-5-yl)-3-hydroxy-1-methylpyrrolidin-2-one and the second eluting product was (S)-3-(2-(3-(4-aminopyrido[3,2-d]pyrimidin-6-yl-2-d)phenyl)oxazol-5-yl)-3-hydroxy-1-methylpyrrolidin-2-one (Example 40). The fractions containing (R)-3-(2-(3-(4-aminopyrido[3,2-d]pyrimidin-6-yl-2-d)phenyl)oxazol-5-yl)-3-hydroxy-1-methylpyrrolidin-2-one were collected and concentrated under reduced pressure. This product was suspended in water (10 mL), the mixture was frozen by insertion into a −78° C. bath, and then lyophilized to dryness to afford (R)-3-(2-(3-(4-aminopyrido[3,2-d]pyrimidin-6-yl-2-d)phenyl)oxazol-5-yl)-3-hydroxy-1-methylpyrrolidin-2-one (22.5 mg, 42%) as a white solid. MS (ESI): Mass calcd. for C₂₁H₁₇DN₆O₃, 403.2; m/z found, 404.1 [M+H]⁺. ¹H NMR (400 MHz, DMSO-d₆) δ 8.77 (s, 1H), 8.62 (d, J=7.9 Hz, 1H), 8.50 (d, J=8.8 Hz, 1H), 8.19 (d, J=8.8 Hz, 1H), 8.14-8.01 (m, 3H), 7.77-7.67 (m, 1H), 7.29 (s, 1H), 6.73-6.31 (m, 1H), 3.51-3.41 (m, 2H), 2.84 (s, 3H), 2.71-2.61 (m, 1H), 2.33-2.22 (m, 1H).

### Example 40: (S)-3-(2-(3-(4-Aminopyrido[3,2-d]pyrimidin-6-yl-2-d)phenyl)oxazol-5-yl)-3-hydroxy-1-methylpyrrolidin-2-one

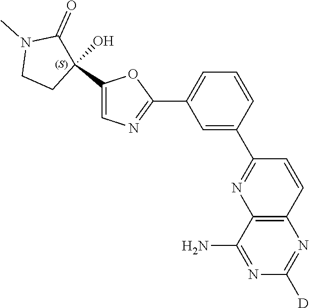

(S)-3-(2-(3-(4-Aminopyrido[3,2-d]pyrimidin-6-yl-2-d)phenyl)oxazol-5-yl)-3-hydroxy-1-methylpyrrolidin-2-one was the second eluting product from Example 39. The fractions containing this product were collected and concentrated under reduced pressure. The product was suspended in water (10 mL), the mixture was frozen by insertion into a −78° C. bath, and then lyophilized to dryness to afford (S)-3-(2-(3-(4-aminopyrido[3,2-d]pyrimidin-6-yl-2-d)phenyl)oxazol-5-yl)-3-hydroxy-1-methylpyrrolidin-2-one (20.6 mg, 38%) as a white solid. MS (ESI): Mass calcd. for C₂₁H₁₇DN₆O₃, 403.2; m/z found, 404.1 [M+H]⁺. ¹H NMR (400 MHz, DMSO-d₆) δ 8.77 (s, 1H), 8.62 (d, J=8.0 Hz, 1H), 8.50 (d, J=8.9 Hz, 1H), 8.19 (d, J=8.8 Hz, 1H), 8.15-8.00 (m, 3H), 7.76-7.68 (m, 1H), 7.29 (s, 1H), 6.68-6.42 (m, 1H), 3.51-3.41 (m, 2H), 2.84 (s, 3H), 2.71-2.62 (m, 1H), 2.32-2.22 (m, 1H).

### Example 41: (R)-3-(2-(3-(4-Aminopyrido[3,2-d]pyrimidin-6-yl-2-d)phenyl)oxazol-4-yl)-3-hydroxy-1-methylpyrrolidin-2-one

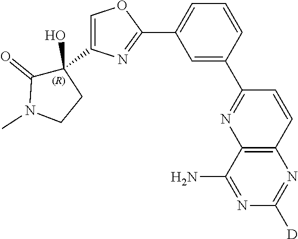

Step A: (R,S)-3-(2-(3-Chlorophenyl)oxazol-4-yl)-3-hydroxy-1-methylpyrrolidin-2-one. n-Butyllithium (1.9 mL, 0.48 mmol, 2.5 M in hexanes) was added dropwise to a solution of 4-bromo-2-(3-chlorophenyl)oxazole (1.0 g, 3.9 mmol) in methylene chloride (10 mL) that had been cooled to −72° C. The resultant mixture was stirred at −72° C. for 20 minutes, treated with a solution of 1-methylpyrrolidine-2,3-dione (530 mg, 4.7 mmol) in methylene chloride (5 mL), and stirred at −72° C. for 2 hours. After this time, the mixture was quenched with saturated aqueous NH₄Cl solution (30 mL), warmed to room temperature, and extracted with ethyl acetate (50 mL×2). The combined organic extracts were washed with brine, dried over anhydrous Na₂SO₄, filtered, and concentrated under reduced pressure. The resulting residue was purified by FCC (eluent: petroleum ether:ethyl acetate=1:0 to 1:2) to afford (R,S)-3-(2-(3-chlorophenyl)oxazol-4-yl)-3-hydroxy-1-methylpyrrolidin-2-one (470 mg, 38%) as a yellow solid. MS (ESI): Mass calcd. for C₁₄H₁₃ClN₂O₃, 292.1; m/z found 293.2 [M+H]⁺.

Step B: (R,S)-3-Hydroxy-1-methyl-3-(2-(3-(4,4,5,5-tetramethyl-1,3,2-dioxaborolan-2-yl)phenyl)oxazol-4-yl)pyrrolidin-2-one. (R,S)-3-(2-(3-Chlorophenyl)oxazol-4-yl)-3-hydroxy-1-methylpyrrolidin-2-one (470 mg, 0.161 mmol), 4,4,4′,4′,5,5,5′,5′-octamethyl-2,2′-bi(1,3,2-dioxaborolane) (815 mg, 3.21 mmol), KOAc (0.94 g, 9.6 mmol), and 1,4-dioxane (10 mL) were added to microwave tube. The resultant mixture was sparged with N₂ for 5 minutes, treated with chloro(2-dicyclohexylphosphino-2′,4′,6′-triisopropyl-1,1′-biphenyl)[2-(2′-amino-1,1′-biphenyl)]palladium(II) (376 mg, 0.478 mmol), sparged with N₂ for another 5 minutes, and then subjected to microwave irradiation at 90° C. for 1 h. After this time, the mixture was cooled to room temperature and filtered. The filtrate was concentrated to afford (R,S)-3-hydroxy-1-methyl-3-(2-(3-(4,4,5,5-tetramethyl-1,3,2-dioxaborolan-2-yl)phenyl)oxazol-4-yl)pyrrolidin-2-one (750 mg) as a black solid. MS (ESI): Mass calcd. for C₂₀H₂₅BN₂O₅, 384.2; m/z found 385.4 [M+H]⁺.

Step C: (R,S)-3-(2-(3-(4-Aminopyrido[3,2-d]pyrimidin-6-yl-2-d)phenyl)oxazol-4-yl)-3-hydroxy-1-methylpyrrolidin-2-one. (R,S)-3-Hydroxy-1-methyl-3-(2-(3-(4,4,5,5-tetramethyl-1,3,2-dioxaborolan-2-yl)phenyl)oxazol-4-yl)pyrrolidin-2-one (750 mg, 1.96 mmol), 6-chloropyrido[3,2-d]pyrimidin-2-d-4-amine (Intermediate 17, 100 mg, 0.60 mmol) and K₃PO₄ (225 mmol, 1.06 mmol) were added to a microwave tube and the resultant mixture dissolved in 1,4-dioxane/H₂O (4:1) (10 mL). This mixture was sparged with N₂ for 5 minutes and then treated with Pd(dtbpf)Cl₂ (36 mg, 0.055 mmol). The mixture was sparged with N₂ for another 5 minutes and then subjected to microwave irradiation at 100° C. for 1 h. After this time, the mixture was cooled to room temperature and filtered. The filtrate was diluted with H₂O (20 mL) and extracted with ethyl acetate (20 mL×2). The combined organic extracts were washed with brine, dried over anhydrous Na₂SO₄, filtered, and concentrated under reduced pressure to give the product, which was purified by preparative HPLC using an HPLC column, such as a Xtimate C18 150×40 mm×5 μm column (eluent: 19% to 39%, H₂O (0.04% NH₄OH+10 mM NH₄HCO₃):CH₃CN). The product was suspended in water (10 mL), the mixture was frozen by insertion into a −78° C. bath, and then lyophilized to dryness to afford (R,S)-3-(2-(3-(4-aminopyrido[3,2-d]pyrimidin-6-yl-2-d)phenyl)oxazol-4-yl)-3-hydroxy-1-methylpyrrolidin-2-one (50 mg, 18%) as a yellow solid. MS (ESI): Mass calcd. for C₂₁H₁₇DN₆O₃, 403.2; m/z found 404.2 [M+H]⁺.

Step D: (R)-3-(2-(3-(4-Aminopyrido[3,2-d]pyrimidin-6-yl-2-d)phenyl)oxazol-4-yl)-3-hydroxy-1-methylpyrrolidin-2-one. The (R) and (S) enantiomers of (R,S)-3-(2-(3-(4-aminopyrido[3,2-d]pyrimidin-6-yl-2-d)phenyl)oxazol-4-yl)-3-hydroxy-1-methylpyrrolidin-2-one (50 mg, 0.124 mmol) were separated by SFC using an SFC column, such as a DAICEL CHIRALCEL® OD-H, 250 mm×30 mm×5 μm column using an isocratic elution of EtOH:supercritical CO₂ (35%:65%). The second eluting enantiomer was (R)-3-(2-(3-(4-aminopyrido[3,2-d]pyrimidin-6-yl-2-d)phenyl)oxazol-4-yl)-3-hydroxy-1-methylpyrrolidin-2-one. The fractions containing this compound were collected and concentrated under reduced pressure. The product was suspended in water (10 mL), the mixture was frozen by insertion into a −78° C. bath, and then lyophilized to dryness to afford (R)-3-(2-(3-(4-aminopyrido[3,2-d]pyrimidin-6-yl-2-d)phenyl)oxazol-4-yl)-3-hydroxy-1-methylpyrrolidin-2-one (14.5 mg, 29%) as a colorless solid. MS (ESI): Mass calcd. for C₂₁H₁₇DN₆O₃, 403.15; m/z found, 404.2 [M+H]⁺. ¹H NMR (400 MHz, DMSO-d₆) δ 8.76 (s, 1H), 8.60 (d, J=7.7 Hz, 1H), 8.53-8.48 (m, 1H), 8.21-8.15 (m, 2H), 8.12-8.01 (m, 3H), 7.74-7.68 (m, 1H), 6.39-6.06 (m, 1H), 3.49-3.45 (m, 2H), 2.84 (s, 3H), 2.66-2.57 (m, 1H), 2.25-2.15 (m, 1H).

### Example 42: (S)-6-(3-(4-(3-Hydroxy-1-methyl-2-oxopyrrolidin-3-yl)-1H-1,2,3-triazol-1-yl)phenyl)picolinamide

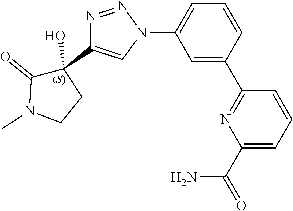

(S)-3-Hydroxy-1-methyl-3-(1-(3-(4,4,5,5-tetramethyl-1,3,2-dioxaborolan-2-yl)phenyl)-1H-1,2,3-triazol-4-yl)pyrrolidin-2-one (Example 36, step B, 165 mg, 0.429 mmol), 6-chloropicolinamide (82 mg, 0.47 mmol), K₃PO₄ (273 mg, 1.29 mmol), 1,4-dioxane (8 mL), and H₂O (2 mL) were added to a microwave tube. The mixture was sparged with Ar for 5 minutes and then treated with Pd(dtbpf)Cl₂ (28 mg, 0.043 mmol). The mixture was sparged with Ar for another 5 minutes and then subjected to microwave irradiation at 90° C. for 1 h. After this time, the mixture was cooled to room temperature and concentrated to dryness under reduced pressure. The resulting residue was initially purified by FCC (eluent: petroleum ether:ethyl acetate=1:0 to 0:1). The product was further purified by preparative HPLC using an HPLC column, such as a Phenomenex Gemini NX-C18, 75 mm×30 mm×3 μm column (eluent: 20% to 50%, CH₃CN and H₂O (with 10 mM NH₄HCO₃)) to afford the product. The product was suspended in water (10 mL), the mixture was frozen by insertion into a −78° C. bath, and then lyophilized to dryness to afford (S)-6-(3-(4-(3-hydroxy-1-methyl-2-oxopyrrolidin-3-yl)-1H-1,2,3-triazol-1-yl)phenyl)picolinamide (20 mg, 12%) as a white solid. MS (ESI): Mass calcd. for C₁₉H₁₈N₆O₃, 378.1; m/z found, 379.2 [M+H]⁺. ¹H NMR (400 MHz, DMSO-d₆) δ 8.96 (s, 1H), 8.75-8.70 (m, 1H), 8.53 (s, 1H), 8.43 (d, J=7.9 Hz, 1H), 8.37 (d, J=7.7 Hz, 1H), 8.16-8.09 (m, 1H), 8.08-8.02 (m, 2H), 7.77 (s, 1H), 7.76-7.71 (m, 1H), 6.30 (s, 1H), 3.54-3.44 (m, 2H), 2.83 (s, 3H), 2.79-2.71 (m, 1H), 2.32-2.22 (m, 1H).

### Example 43. (R)-3-(5-(3-(4-Aminopyrido[3,2-d]pyrimidin-6-yl-2-d)phenyl)-1-methyl-1H-pyrazol-3-yl)-3-hydroxy-1-methylpyrrolidin-2-one

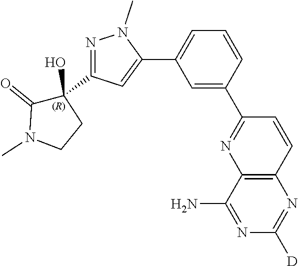

Step A: 3,5-Dibromo-1-methyl-1H-pyrazole. Sodium hydride (2.51 g, 60 wt. % in mineral oil, 62.8 mmol) was added to a solution of 3,5-dibromo-1H-pyrazole (7.00 g, 31.0 mmol) and THF (80 mL) that had been cooled to 0° C. The mixture was then stirred at 0° C. for 1 hour, treated with methyl iodidie (15.6 g, 110 mmol) dropwise, and then stirred for 2 h with gradual warming to room temperature. After this time, the mixture was quenched with saturated aqueous NH₄Cl solution (100 mL), and extracted with ethyl acetate (200 mL×3). The combined organic extracts were washed with brine (30 mL), dried over anhydrous Na₂SO₄, filtered, and concentrated to dryness under reduced pressure. The resulting residue was purified by FCC (eluent: petroleum ether:ethyl acetate=1:0 to 1:1) to afford 3,5-dibromo-1-methyl-1H-pyrazole (7.0 g, 84%) as a yellow oil. MS (ESI): Mass calcd. for C₄H₄Br₂N₂, 237.9; m/z found, 238.8 [M+H]⁺.

Step B: 3-Bromo-5-(3-chlorophenyl)-1-methyl-1H-pyrazole. 3,5-Dibromo-1-methyl-1H-pyrazole (7.00 g, 29.0 mmol) was added to a mixture of (3-chlorophenyl)boronic acid (3.88 g, 24.8 mmol), K₂CO₃ (12.1 g, 87.5 mmol), 1,4-dioxane (100 mL), and H₂O (20 mL). The mixture was sparged with Ar for 5 minutes and then treated with Pd(dppf)Cl₂ (2.14 g, 2.93 mmol). The mixture was sparged with Ar for another 5 minutes and then heated at 105° C. for 16 h. After this time, the mixture was cooled to room temperature, poured into H₂O (30 mL), and extracted with ethyl acetate (30 mL×3). The combined organic extracts were washed with brine (10 mL), dried over anhydrous Na₂SO₄, filtered, and concentrated to dryness under reduced pressure to give the product, which was initially purified by FCC (eluent: petroleum ether:ethyl acetate=1:0 to 3:1). The product was further purified by preparative HPLC using an HPLC column, such as an Xtimate C18, 150×40 mm×5 μm column (eluent: 45% to 75%, CH₃CN and H₂O (with 0.05% NH₄OH)) to afford product. The product was suspended in water (10 mL), the mixture was frozen by insertion into a −72° C. bath, and then lyophilized to dryness to afford 3-bromo-5-(3-chlorophenyl)-1-methyl-1H-pyrazole (2.5 g, 30%) as a white solid. MS (ESI): Mass calcd. for C₁₀H₈BrClN₂, 270.0; m/z found, 271.0 [M+H]⁺. ¹H NMR (400 MHz, DMSO-d₆) δ 7.65-7.63 (m, 1H), 7.56-7.51 (m, 3H), 6.64 (s, 1H), 3.83 (s, 3H).

Step C: 3-(5-(3-Chlorophenyl)-1-methyl-1H-pyrazol-3-yl)-3-hydroxy-1-methylpyrrolidin-2-one. n-BuLi (1.62 mL, 4.05 mmol, 2.5 M in hexanes) was added dropwise to a solution of 3-bromo-5-(3-chlorophenyl)-1-methyl-1H-pyrazole (1.0 g, 3.7 mmol) and methylene chloride (15 mL) that had been cooled to −70° C. under a N₂ atmosphere. The resultant mixture was stirred at −70° C. for 0.5 hour before it was treated with a solution of 1-methylpyrrolidine-2,3-dione (500 mg, 4.4 mmol) in methylene chloride (5 mL). The resultant mixture was stirred at −70° C. for another 1 h. After this time, the mixture was quenched with saturated aqueous NH₄Cl solution (50 mL), warmed to room temperature, and extracted with ethyl acetate (80 mL×3). The combined organic extracts were dried over anhydrous Na₂SO₄, filtered, and concentrated to dryness under reduced pressure. The resulting residue was purified by FCC (eluent: petroleum ether:ethyl acetate=0:1 to 0:1) to afford 3-(5-(3-chlorophenyl)-1-methyl-1H-pyrazol-3-yl)-3-hydroxy-1-methylpyrrolidin-2-one (500 mg, 44%) as a yellow solid. MS (ESI): Mass calcd. for C₁₅H₁₆ClN₃O₂, 305.1; m/z found, 306.1 [M+H]⁺. ¹H NMR (400 MHz, DMSO-d₆) δ 7.59-7.57 (m, 1H), 7.54-7.47 (m, 3H), 7.54-7.47 (m, 1H), 6.49 (s, 1H), 3.80 (s, 3H), 3.39-3.35 (m, 2H), 2.76 (s, 3H), 2.69-2.62 (m, 1H), 2.16-2.08 (m, 1H).

Step D: 3-Hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one. Chloro(2-dicyclohexylphosphino-2′,4′,6′-triisopropyl-1,1′-biphenyl)[2-(2′-amino-1,1′-biphenyl)]palladium(II) (129 mg, 0.16 mmol) was added to a mixture of 3-(5-(3-chlorophenyl)-1-methyl-1H-pyrazol-3-yl)-3-hydroxy-1-methylpyrrolidin-2-one (500 mg, 1.6 mmol), 4,4,4′,4′,5,5,5′,5′-octamethyl-2,2′-bi(1,3,2-dioxaborolane) (831 mg, 3.27 mmol), KOAc (481 mg, 4.90 mmol), and 1,4-dioxane (30 mL) under a N₂ atmosphere. The resultant mixture was heated at 110° C. for 6 h. After this time, the mixture was cooled to room temperature and concentrated to dryness under reduced pressure. The resulting residue was purified by FCC (eluent: petroleum ether:ethyl acetate=1:0 to 0:1) to afford 3-hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one (600 mg, 41%) as a brown oil. MS (ESI): Mass calcd. for C₂₁H₂₈BN₃O₄, 397.2; m/z found, 398.2 [M+H]⁺.

Step E: (R,S)-3-(5-(3-(4-Aminopyrido[3,2-d]pyrimidin-6-yl-2-d)phenyl)-1-methyl-1H-pyrazol-3-yl)-3-hydroxy-1-methylpyrrolidin-2-one. 6-Chloropyrido[3,2-d]pyrimidin-2-d-4-amine (Intermediate 17, 70 mg, 0.4 mmol), 3-hydroxy-1-methyl-3-(1-methyl-5-(3-(4,4,5,5-tetramethyl-1,3,2-dioxaborolan-2-yl)phenyl)-1H-pyrazol-3-yl)pyrrolidin-2-one (200 mg, 0.50 mmol), and K₃PO₄ (240 mg, 1.13 mmol) were added to a microwave tube and dissolved in 1,4-dioxane (10 mL) and H₂O (2.5 mL). The resultant mixture was sparged with Ar for 5 minutes and then treated with Pd(dtbpf)Cl₂ (25 mg, 0.04 mmol). The mixture was sparged with Ar for another 5 minutes and then subjected to microwave irradiation at 80° C. for 1 h. After this time, the mixture was cooled to room temperature. The reaction mixture was combined with another batch and concentrated to dryness under reduced pressure to afford the product, which was purified by preparative HPLC using an HPLC column, such as a Boston Prime C18, 150×30 mm×5 μm column (eluent: 20% to 50%, CH₃CN and H₂O with (0.04% NH₄OH+10 mM NH₄HCO₃)) to afford (R,S)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one. This racemic product was suspended in water (10 mL), the mixture was frozen by insertion into a −78° C. bath, and then concentrated to dryness to afford (R,S)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one (110 mg, 66%) as a white solid.

Step F. (R)-3-(5-(3-(4-Aminopyrido[3,2-d]pyrimidin-6-yl-2-d)phenyl)-1-methyl-1H-pyrazol-3-yl)-3-hydroxy-1-methylpyrrolidin-2-one. The enantiomers of (R,S)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one were separated by SFC using an SFC column, such as a DAICEL CHIRALPAK® AD 250 mm×30 mm, 10 m column (isocratic elution: i-PrOH (containing 0.1% of 25% aqueous NH₃ solution): supercritical CO₂, from 55%:45%) to provide the first eluting enantiomer ((R)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one) and the second eluting enantiomer ((S)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one) (Example 44). The fractions containing (R)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one were collected and concentrated under reduced pressure. Then, it was suspended in water (10 mL) and the mixture was frozen by insertion into a −78° C. bath and then lyophilized to dryness to afford (R)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one (23.8 mg, 21.6%) as a white solid. MS (ESI): Mass calcd. for C₂₂H₂₀DN₇O₂, 416.2; m/z found, 417.1 [M+H]⁺. ¹H NMR (400 MHz, CDCl₃) δ 8.21-8.16 (m, 1H), 8.12-8.02 (m, 3H), 7.60-7.54 (m, 1H), 7.51-7.42 (m, 1H), 7.13 (br s, 1H), 6.78 (br s, 1H), 6.51 (s, 1H), 5.01 (br s, 1H), 3.87 (s, 3H), 3.61-3.53 (m, 1H), 3.52-3.44 (m, 1H), 2.97 (s, 3H), 2.91-2.84 (m, 1H), 2.50-2.40 (m, 1H).

### Example 44: (S)-3-(5-(3-(4-Aminopyrido[3,2-d]pyrimidin-6-yl-2-d)phenyl)-1-methyl-1H-pyrazol-3-yl)-3-hydroxy-1-methylpyrrolidin-2-one

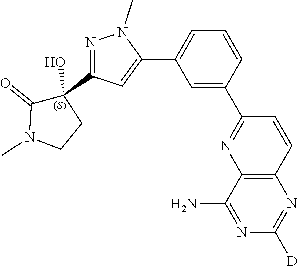

(S)-3-(5-(3-(4-Aminopyrido[3,2-d]pyrimidin-6-yl-2-d)phenyl)-1-methyl-1H-pyrazol-3-yl)-3-hydroxy-1-methylpyrrolidin-2-one was the second eluting product from Example 43. The fractions containing this compound were collected and concentrated under reduced pressure. The product was suspended in water (10 mL), the mixture was frozen by insertion into a −78° C. bath, and then lyophilized to dryness to afford (S)-3-(5-(3-(4-aminopyrido[3,2-d]pyrimidin-6-yl-2-d)phenyl)-1-methyl-1H-pyrazol-3-yl)-3-hydroxy-1-methylpyrrolidin-2-one (50.1 mg) as a white solid. MS (ESI): Mass calcd. for C₂₂H₂₀DN₇O₂, 416.2; m/z found, 417.1 [M+H]⁺. ¹H NMR (400 MHz, CDCl₃) δ 8.24-8.18 (m, 1H), 8.16-8.05 (m, 3H), 7.64-7.56 (m, 1H), 7.52-7.47 (m, 1H), 7.10 (br.s, 1H), 6.50 (s, 1H), 6.36 (br s, 1H), 4.42 (br s, 1H), 3.88 (s, 3H), 3.61-3.53 (m, 1H), 3.51-3.44 (m, 1H), 2.97 (s, 3H), 2.91-2.83 (m, 1H), 2.50-2.40 (m, 1H).

### Example 45: (R)-3-Amino-6-(3-(5-(3-hydroxy-1-methyl-2-oxopyrrolidin-3-yl)isoxazol-3-yl)phenyl)-4-methylpicolinamide

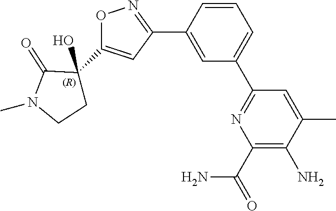

Step A: Methyl (R)-3-amino-6-(3-(5-(3-hydroxy-1-methyl-2-oxopyrrolidin-3-yl)isoxazol-3-yl)phenyl)-4-methylpicolinate. The mixture of methyl 3-amino-6-chloro-4-methylpicolinate (26.0 mg, 0.13 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 50.0 mg, 0.13 mmol), tri-tert-butylphosphine (10.0 mg, 0.05 mmol), tris(dibenzylideneacetone)dipalladium(0) (24.0 mg, 0.026 mmol), and potassium fluoride (7.6 mg, 0.13 mmol) in 1,4-dioxane/H₂O (1.0 mL/1.0 mL) was degassed for 10 min with nitrogen. Then the mixture was heated at 90° C. for 1.5 h. The mixture was partitioned with H₂O (1 mL) and extracted with EtOAc (3×2 mL). The organic extracts were combined and concentrated. The residue was purified by silica gel chromatography (5-10% MeOH/DCM) to provide methyl (R)-3-amino-6-(3-(5-(3-hydroxy-1-methyl-2-oxopyrrolidin-3-yl)isoxazol-3-yl)phenyl)-4-methylpicolinate (15 mg). MS (ESI): Mass calcd. for C₂₂H₂₂N₄O₅, 422.1; m/z found, 423.1 [M+H]⁺.

Step B: (R)-3-Amino-6-(3-(5-(3-hydroxy-1-methyl-2-oxopyrrolidin-3-yl)isoxazol-3-yl)phenyl)-4-methylpicolinamide. To methyl (R)-3-amino-6-(3-(5-(3-hydroxy-1-methyl-2-oxopyrrolidin-3-yl)isoxazol-3-yl)phenyl)-4-methylpicolinate (15 mg, 0.035 mmol) in a vial was added NH₃ in MeOH (2 M, 5 mL). The mixture was heated at 60° C. for 24 h. The mixture was concentrated and purified by silica gel chromatography (5-10% MeOH/DCM) to provide (R)-3-amino-6-(3-(5-(3-hydroxy-1-methyl-2-oxopyrrolidin-3-yl)isoxazol-3-yl)phenyl)-4-methylpicolinamide (4.0 mg, 27%) as a solid. MS (ESI): Mass calcd. for C₂₁H₂₁N₅O₄, 407.1; m/z found, 408.1 [M+H]⁺. ¹H NMR (500 MHz, CD₃OD) δ 8.51-8.45 (m, 1H), 8.15-8.02 (m, 1H), 7.87-7.74 (m, 2H), 7.61-7.47 (m, 1H), 7.00 (s, 1H), 3.62-3.54 (m, 2H), 2.97 (s, 3H), 2.81-2.70 (m, 1H), 2.48-2.36 (m, 1H), 2.31 (s, 3H).

### Example 46: (R)-3-Amino-6-(3-(5-(3-hydroxy-1-methyl-2-oxopyrrolidin-3-yl)isoxazol-3-yl)phenyl)-4-methoxypicolinamide

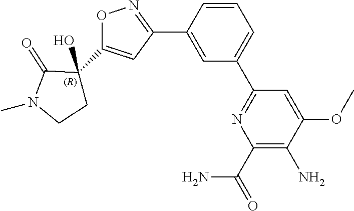

Step A: 3-Amino-4-chloro-6-methoxypicolinamide. To 3-amino-4,6-dichloropicolinonitrile (188 mg, 1.00 mmol) in DMA (0.5 mL), NaOMe (0.46 mmol, 2.00 mmol, 25% wt in MeOH) was added. The mixture was heated at 50° C. for 16 h. Saturated aqueous NH₄Cl solution (1 mL) was added and solids were formed. These solids were collected by filtration, washed with DCM (2×2 mL) and dried to afford 3-amino-4-chloro-6-methoxypicolinamide as a solid (110 mg, 55%). MS (ESI): Mass calcd. for C₂₁H₂₁N₅O₅, 201.1; m/z found, 202.1 [M+H]⁺. ¹H NMR (500 MHz, CD₃OD) δ 6.91 (s, 1H), 3.96 (s, 3H).

Step B: (R)-3-Amino-6-(3-(5-(3-hydroxy-1-methyl-2-oxopyrrolidin-3-yl)isoxazol-3-yl)phenyl)-4-methoxypicolinamide. The mixture of 3-amino-4-chloro-6-methoxypicolinamide (52.0 mg, 0.26 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 100 mg, 0.26 mmol), tri-tert-butylphosphine (52.0 mg, 0.26 mmol), tris(dibenzylideneacetone)dipalladium(0) (119 mg, 0.13 mmol), and potassium fluoride (181 mg, 3.0 mmol) in 1,4-dioxane/H₂O (2.0 mL/1.0 mL) was degassed for 10 min with nitrogen. Then the mixture was heated at 90° C. for 1.5 h. The mixture was cooled to room temperature, H₂O (1 mL) was added and the resulting solution was extracted with EtOAc (3×2 mL). The organic extracts were combined and concentrated. The residue was purified by silica gel chromatography (5-10% MeOH/DCM) to yield (R)-3-amino-6-(3-(5-(3-hydroxy-1-methyl-2-oxopyrrolidin-3-yl)isoxazol-3-yl)phenyl)-4-methoxypicolinamide (13 mg, 12%) as a solid. MS (ESI): Mass calcd. for C₂₁H₂₁N₅O₅, 423.1; m/z found, 424.1 [M+H]⁺. ¹H NMR (500 MHz, CD₃OD) δ 8.48 (t, J=1.8 Hz, 1H), 8.18-8.05 (m, 1H), 7.83-7.74 (m, 1H), 7.56 (t, J=7.8 Hz, 1H), 7.47 (s, 1H), 7.01 (s, 1H), 4.06 (s, 3H), 3.60-3.55 (m, 2H), 2.96 (s, 3H), 2.78-2.71 (m, 1H), 2.44-2.37 (m, 1H).

### Example 47: (R)-4-(3-(5-(3-Hydroxy-1-methyl-2-oxopyrrolidin-3-yl)isoxazol-3-yl)phenyl)-6-methoxypicolinamide

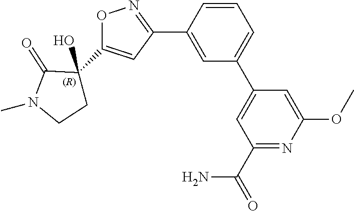

Step A: 4-Chloro-6-methoxypicolinamide. To methyl 4-chloro-6-methoxypicolinate (200 mg, 0.99 mmol) in a vial, was added NH₃ (5 mL, 2 M in MeOH). The mixture was heated at 60° C. for 24 h. The mixture was then concentrated to provide 4-chloro-6-methoxypicolinamide (185 mg, 100%). MS (ESI): Mass calcd. for C₇H₇ClN₂O₂, 186.0; m/z found, 187.0 [M+H]⁺. ¹H NMR (500 MHz, CD₃OD) δ 7.68 (d, J=1.6 Hz, 1H), 7.05 (d, J=1.8 Hz, 1H), 4.01 (s, 3H).

Step B: (R)-4-(3-(5-(3-Hydroxy-1-methyl-2-oxopyrrolidin-3-yl)isoxazol-3-yl)phenyl)-6-methoxypicolinamide. 4-Chloro-6-methoxypicolinamide (74.0 mg, 0.40 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 168 mg, 0.44 mmol), bis(triphenylphosphine)palladium(ii) chloride (45 mg, 0.07 mmol) and K₂CO₃ (1.2 mL, 2.5 mmol, 2 M in H₂O) in 1,4-dioxane/EtOH (1.4 mL/1.0 mL) was degassed for 10 min with nitrogen. Then the mixture was heated at 170° C. for 10 min and then allowed to cool to room temperature. The mixture was partitioned with EtOAc/H₂O (2 mL/2 mL) and extracted with EtOAc (3×2 mL). The organic extracts were combined and concentrated. The residue was purified by silica gel chromatography (10% MeOH in DCM) to yield (R)-4-(3-(5-(3-hydroxy-1-methyl-2-oxopyrrolidin-3-yl)isoxazol-3-yl)phenyl)-6-methoxypicolinamide (30 mg, 18%) as a solid. MS (ESI): Mass calcd. for C₂₁H₂₀N₄O₅, 408.1; m/z found, 409.1 [M+H]⁺. ¹H NMR (500 MHz, CD₃OD) 8.20 (d, J=2.0 Hz, 1H), 8.05 (d, J=1.5 Hz, 1H), 8.00-7.90 (m, 1H), 7.90-7.79 (m, 1H), 7.64 (t, J=7.8 Hz, 1H), 7.30 (t, J=1.4 Hz, 1H), 6.99 (s, 1H), 4.05 (s, 3H), 3.61-3.54 (m, 2H), 2.96 (s, 3H), 2.79-2.70 (m, 1H), 2.46-2.34 (m, 1H).

### Example 48: (R)-3-Hydroxy-3-(3-(3-(4-methoxy-1-methyl-1H-pyrazolo[3,4-b]pyridin-6-yl)phenyl)isoxazol-5-yl)-1-methylpyrrolidin-2-one

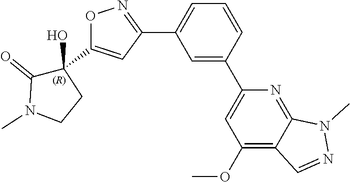

(R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 45 mg, 0.12 mmol) in freshly distilled DMF (1 mL) was evacuated and argon was bubbled through the solution. 6-Chloro-4-methoxy-1-methyl-1H-pyrazolo[3,4-b]pyridine (27 mg, 0.13 mmol), butyldi(1-adamantanyl)phosphine (4.6 mg, 0.012 mmol), and methanesulfonato-2′-methylamino-1,1′-biphenyl-2-yl-palladacycles generation 4 (RuPhos Pd G4, (CAS #1599466-85-9), 5.1 mg, 0.0060 mmol) were added and the mixture was heated at 80° C. for 12 h. The mixture was then cooled to room temperature and a precipitate formed. The precipitate was filtered off and discarded and the filtrate concentrated. The product was purified by HPLC using an HPLC column such as, a SUNFIRE® C18 100×20 mm 5 m to provide Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, as a solid (15 mg, 29%). MS (ESI): Mass calcd. for C₂₂H₂₁N₅O₄, 419.6; m/z found, 420.5 [M+H]⁺. ¹H NMR (500 MHz, CD₃CN) δ 8.07 (s, 1H), 7.80 (d, J=7.9 Hz, 1H), 7.58 (s, 1H), 7.43 (d, J=7.7 Hz, 1H), 7.12 (t, J=7.8 Hz, 1H), 6.81 (s, 1H), 6.60 (s, 1H), 6.19 (s, 1H), 3.62 (s, 3H), 3.54 (s, 3H), 2.99-2.84 (m, 2H), 2.29 (s, 3H), 2.08-2.02 (m, 1H), 1.78-1.75 (m, 1H).

### Example 49: (R)-3-(3-(3-(1H-Pyrrolo[2,3-b]pyridin-4-yl)phenyl)isoxazol-5-yl)-3-hydroxy-1-methylpyrrolidin-2-one

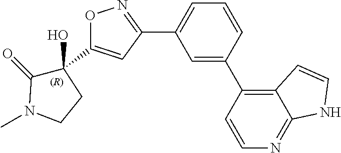

(R)-3-(3-(3-(1H-Pyrrolo[2,3-b]pyridin-4-yl)phenyl)isoxazol-5-yl)-3-hydroxy-1-methylpyrrolidin-2-one (22.7 mg) was prepared in a manner analogous to Example 48 using 4-bromo-1H-pyrrolo[2,3-b]pyridine instead on 6-chloro-4-methoxy-1-methyl-1H-pyrazolo[3,4-b]pyridine. MS (ESI): Mass calcd. for C₂₁H₁₈N₄O₃, 374.1; m/z found, 375.2 [M+H]⁺. ¹H NMR (500 MHz, CD₃CN) δ 9.95 (s, 1H), 8.36 (d, J=4.9 Hz, 1H), 8.19 (s, 1H), 7.96 (d, J=7.8 Hz, 1H), 7.91 (d, J=7.7 Hz, 1H), 7.68 (t, J=7.7 Hz, 1H), 7.49 (d, J=3.6 Hz, 1H), 7.29 (d, J=4.9 Hz, 1H), 6.90 (s, 1H), 6.70 (d, J=3.6 Hz, 1H), 4.75 (s, 1H), 3.51 (t, J=6.6 Hz, 2H), 2.91 (s, 3H), 2.69 (d, J=5.7 Hz, 1H), 2.38 (d, J=7.5 Hz, 1H).

### Example 50: (R)-3-Amino-6-(3-(5-(3-hydroxy-1-methyl-2-oxopyrrolidin-3-yl)isoxazol-3-yl)phenyl)-4-(trifluoromethyl)picolinamide

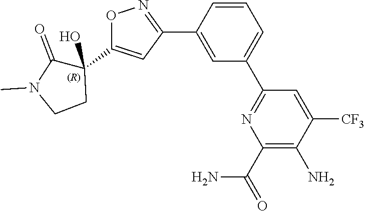

Step A: 3-Amino-6-chloro-4-(trifluoromethyl)picolinonitrile. (1,10-Phenanthroline)(trifluoromethyl)copper(I) (1.62 g, 5.18 mmol) was added to a mixture of 3-amino-4-bromo-6-chloropicolinonitrile (1.0 g, 4.3 mmol) in DMF (20 mL). This mixture was sparged with N₂ for 5 minutes and then heated at 100° C. for 16 h. After this time, the mixture was cooled to room temperature, poured into H₂O (20 mL), and extracted with ethyl acetate (40 mL×3). The combined organic solvent extracts were washed with brine, dried over anhydrous Na₂SO₄, filtered, and concentrated under reduced pressure. The resulting residue was purified by FCC (eluent: ethyl acetate:petroleum ether=0:1 to 1:3) to afford 3-amino-6-chloro-4-(trifluoromethyl)picolinonitrile (150 mg, 15% yield) as a yellow solid. MS (ESI): Mass calcd. for C₇H₃ClF₃N₃, 221.0; m/z found, 222.1 [M+H]⁺.

Step B: 3-Amino-6-chloro-4-(trifluoromethyl)picolinamide. Hydrogen peroxide (0.27 mL, 30 wt. % in H₂O, 2.73 mmol) was added to a mixture of 3-amino-6-chloro-4-(trifluoromethyl)picolinonitrile (150 mg, 0.68 mmol), K₂CO₃ (37.5 mg, 0.27 mmol), and DMF (3 mL). This mixture was heated at 30° C. for 48 h. After this time, the mixture was cooled to room temperature, diluted with saturated aqueous Na₂S₂SO₃ solution (5 mL) and extracted with ethyl acetate (20 mL×3). The combined organic solvent extracts were washed with brine, dried over anhydrous Na₂SO₄, filtered, and concentrated under reduced pressure to afford 3-amino-6-chloro-4-(trifluoromethyl)picolinamide (160 mg, 91% yield) as a yellow solid. MS (ESI): Mass calcd. for C₇H₅ClF₃N₃O, 239.0; m/z found, 240.0 [M+H]⁺. ¹H NMR (400 MHz, DMSO-d₆) δ 8.00 (br s, 1H), 7.75-7.64 (m, 2H), 7.49 (br s, 2H).

Step C: (R)-3-Amino-6-(3-(5-(3-hydroxy-1-methyl-2-oxopyrrolidin-3-yl)isoxazol-3-yl)phenyl)-4-(trifluoromethyl)picolinamide. 3-Amino-6-chloro-4-(trifluoromethyl)picolinamide (150 mg, 0.63 mmol) was added to a mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 263 mg, 0.680 mmol), K₃PO₄ (398 mg, 1.88 mmol), and 1,4-dioxane/H₂O (v/v=4/1, 8 mL). The resultant mixture was sparged with N₂ for 5 minutes and then treated with Pd(dtbpf)Cl₂ (38 mg, 0.060 mmol). This mixture was then sparged with N₂ for another 5 minutes and subjected to microwave irradiation at 80° C. for 1 h. After this time, the mixture was cooled to room temperature and filtered. The filtrate was concentrated under reduced pressure to afford the product, which was purified by preparative HPLC using an HPLC column, such as a Phenomenex Gemini-NX 150×30 mm×5 m column (eluent: 34% to 62% CH₃CN in H₂O (with 0.05% NH₃H₂O)). The product was then suspended in water (5 mL), the mixture was frozen at −78° C., and then lyophilized to afford (R)-3-amino-6-(3-(5-(3-hydroxy-1-methyl-2-oxopyrrolidin-3-yl)isoxazol-3-yl)phenyl)-4-(trifluoromethyl)picolinamide (98 mg, 34% yield) as a colorless solid. MS (ESI): Mass calcd. for C₂₁H₁₈F₃N₅O₄, 461.1; m/z found, 462.0 [M+H]⁺. ¹H NMR (400 MHz, DMSO-d₆) δ 8.49 (s, 1H), 8.51-8.47 (m, 1H), 8.47-8.42 (m, 1H), 8.39-8.32 (m, 1H), 8.25 (s, 1H), 7.93-7.87 (m, 1H), 7.80 (br s, 1H), 7.62-7.55 (m, 2H), 7.28 (s, 1H), 6.74 (s, 1H), 3.55-3.41 (m, 2H), 2.85 (s, 3H), 2.63-2.54 (m, 1H), 2.37-2.23 (m, 1H).

### Example 51: (R)-3-Hydroxy-3-(3-(3-(imidazo[1,5-a]pyridin-6-yl)phenyl)isoxazol-5-yl)-1-methylpyrrolidin-2-one

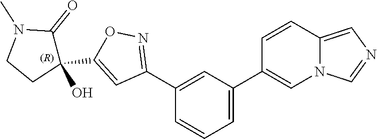

(R)-3-Hydroxy-3-(3-(3-(imidazo[1,5-a]pyridin-6-yl)phenyl)isoxazol-5-yl)-1-methylpyrrolidin-2-one was synthesized in a manner analogous to the synthesis of Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromoimidazo[1,5-a]pyridine in place of 6-chloro-4-methoxy-1-methyl-1H-pyrazolo[3,4-b]pyridine, to give the title compound as a solid (10.8 mg). Purification: RP-HPLC using column: SunFire C18 100×19 mm, 5 m). MS (ESI): Mass calcd. for C₂₁H₁₈N₄O₃ 374.14, m/z found, 375.2 [M+H]⁺. ¹H NMR (600 MHz, DMSO-d₆) δ 8.82 (d, J=1.4 Hz, 1H), 8.38 (s, 1H), 8.15 (d, J=1.8 Hz, 1H), 7.90-7.84 (m, 1H), 7.86-7.78 (m, 1H), 7.65 (d, J=9.5 Hz, 1H), 7.60 (t, J=7.8 Hz, 1H), 7.38 (s, 1H), 7.26-7.20 (m, 1H), 7.14 (s, 1H), 6.70 (s, 1H), 3.52-3.45 (m, 1H), 3.45-3.35 (m, 1H), 2.82 (s, 3H), 2.57-2.53 (m, 1H), 2.30-2.23 (m, 1H).

### Example 52: (R)-3-Hydroxy-1-methyl-3-(3-(3-(pyrrolo[2,1-f][1,2,4]triazin-4-yl)phenyl)isoxazol-5-yl)pyrrolidin-2-one

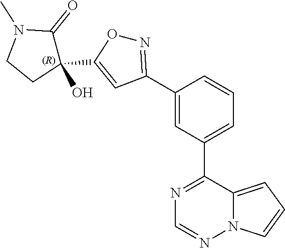

(R)-3-Hydroxy-1-methyl-3-(3-(3-(pyrrolo[2,1-f][1,2,4]triazin-4-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-chloropyrrolo[2,1-f][1,2,4]triazine in place of 6-chloro-4-methoxy-1-methyl-1H-pyrazolo[3,4-b]pyridine, to give the title compound as a solid (15.1 mg). Purification: RP-HPLC SunFire 100×19 mm, 5 m. MS (ESI): Mass calcd. for C₂₀H₁₇N₅O₃ 290.2, m/z found, 291.3 [M+H]⁺. ¹H NMR (500 MHz, Acetonitrile-d₃) δ 8.56 (d, J=11.5 Hz, 2H), 8.24 (d, J=7.8 Hz, 1H), 8.10 (d, J=7.7 Hz, 1H), 8.00 (d, J=2.7 Hz, 1H), 7.74 (t, J=7.8 Hz, 1H), 7.19 (d, J=4.7 Hz, 1H), 7.12-7.08 (m, 1H), 6.91 (s, 1H), 4.55 (s, 1H), 3.55-3.48 (m, 2H), 2.91 (s, 3H), 2.69 (d, J=5.7 Hz, 1H), 2.37 (d, J=7.6 Hz, 1H).

### Example 53: (R)-3-(3-(3-(2-Aminopyridin-4-yl)phenyl)isoxazol-5-yl)-3-hydroxy-1-methylpyrrolidin-2-one

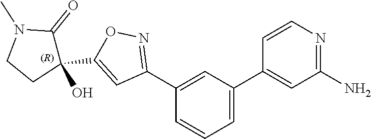

(R)-3-(3-(3-(2-Aminopyridin-4-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-bromopyridin-2-amine in place of 6-chloro-4-methoxy-1-methyl-1H-pyrazolo[3,4-b]pyridine, to give the title compound as a solid (14.4 mg). Purification: RP HPLC; column SunFire C18 100×19 mm, 5 m. MS (ESI): Mass calcd. for C₁₉H₁₈N₄O₃ 350.14, m/z found, 351.2 [M+H]⁺. ¹H NMR (600 MHz, DMSO-d₆) δ 8.07 (d, J=2.0 Hz, 1H), 7.97 (d, J=5.3 Hz, 1H), 7.90 (d, J=7.8 Hz, 1H), 7.74 (d, J=8.2 Hz, 1H), 7.60 (t, J=7.7 Hz, 2H), 7.10 (s, 1H), 6.88-6.82 (m, 1H), 6.75 (d, J=1.6 Hz, 1H), 6.68 (s, 1H), 5.98 (s, 2H), 3.52-3.34 (m, 2H), 2.81 (s, 3H), 2.30-2.20 (m, 1H).

### Example 54: (R)—N-(4-(3-(5-(3-Hydroxy-1-methyl-2-oxopyrrolidin-3-yl)isoxazol-3-yl)phenyl)pyridin-2-yl)acetamide

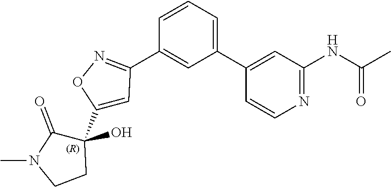

(R)—N-(4-(3-(5-(3-Hydroxy-1-methyl-2-oxopyrrolidin-3-yl)isoxazol-3-yl)phenyl)pyridin-2-yl)acetamide was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using N-(4-chloropyridin-2-yl)acetamide in place of 6-chloro-4-methoxy-1-methyl-1H-pyrazolo[3,4-b]pyridine, to give the title compound as a solid (13.7 mg). Purification: RP HPLC; column SunFire C18 100×19 mm, 5 m. MS (ESI): Mass calcd. for C₂₁H₂₀N₄O₄ 392.2, m/z found, 393.2 [M+H]⁺. ¹H NMR (500 MHz, Acetonitrile-d₃) δ 8.84 (s, 1H), 8.48 (s, 1H), 8.36 (d, J=5.2 Hz, 1H), 8.13 (d, J=2.0 Hz, 1H), 7.95 (d, J=7.8 Hz, 1H), 7.91-7.75 (m, 1H), 7.64 (t, J=7.8 Hz, 1H), 7.58-7.46 (m, 1H), 7.48-7.40 (m, 1H), 4.66 (s, 1H), 3.50 (d, J=5.4 Hz, 2H), 2.90 (d, J=5.6 Hz, 3H), 2.67 (d, J=5.6 Hz, 1H), 2.37 (d, J=7.9 Hz, 1H), 2.18 (s, 3H).

### Example 55: (R)-3-(3-(3-(1-Ethyl-1H-pyrazolo[3,4-b]pyridin-5-yl)phenyl)isoxazol-5-yl)-3-hydroxy-1-methylpyrrolidin-2-one

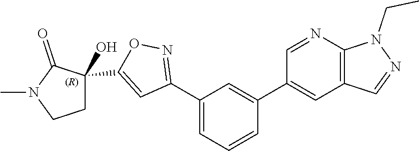

(R)-3-(3-(3-(1-Ethyl-1H-pyrazolo[3,4-b]pyridin-5-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromo-1-ethyl-1H-pyrazolo[3,4-b]pyridine in place of 6-chloro-4-methoxy-1-methyl-1H-pyrazolo[3,4-b]pyridine, to give the title compound as a solid (22 mg). Purification: RP HPLC; column SunFire C18 100×19 mm, 5 m. MS (ESI): Mass calcd. for C₂₂H₂₁₅O₃ 403.2, m/z found 404.0 [M+H]⁺. ¹H NMR (600 MHz, DMSO-d₆) δ 8.94 (d, J=2.2 Hz, 1H), 8.59 (d, J=2.2 Hz, 1H), 8.21 (d, J=3.9 Hz, 2H), 7.94-7.79 (m, 2H), 7.62 (t, J=7.7 Hz, 1H), 7.17 (s, 1H), 6.69 (s, 1H), 4.52 (q, J=7.2 Hz, 2H), 3.43 (dddd, J=32.0, 9.8, 7.7, 5.1 Hz, 2H), 3.26 (s, 1H), 2.82 (s, 3H), 2.26 (ddd, J=13.6, 8.1, 5.7 Hz, 1H), 1.44 (t, J=7.3 Hz, 3H).

### Example 56: (R)-2-(3-(5-(3-hydroxy-1-methyl-2-oxopyrrolidin-3-yl)isoxazol-3-yl)phenyl)-6,7-dihydrofuro[3,2-c]pyridin-4(5H)-one

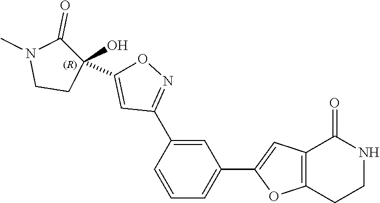

(R)-2-(3-(5-(3-hydroxy-1-methyl-2-oxopyrrolidin-3-yl)isoxazol-3-yl)phenyl)-6,7-dihydrofuro[3,2-c]pyridin-4(5H)-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 2-bromo-4H,5H,6H,7H-furo[3,2-c]pyridin-4-one in place of 6-chloro-4-methoxy-1-methyl-1H-pyrazolo[3,4-b]pyridine, to give the title compound as a solid (8.0 mg). Purification: RP HPLC; column: SunFire C18 100×18 mm, 5 m). MS (ESI): Mass calcd. for C₂₁H₁₉N₃O₅ 393.1, m/z found, 394.2 [M+H]⁺. ¹H NMR (600 MHz, DMSO-d₆) δ 8.20 (d, J=1.7 Hz, 1H), 7.83 (d, J=1.7 Hz, 1H), 7.82-7.74 (m, 1H), 7.54 (t, J=7.8 Hz, 1H), 7.40 (s, 1H), 7.33 (s, 1H), 7.13 (s, 1H), 6.69 (s, 1H), 3.47 (dt, J=7.4, 3.6 Hz, 2H), 3.46-3.42 (m, 1H), 3.40 (dd, J=7.6, 5.7 Hz, 1H), 2.98 (t, J=7.1 Hz, 2H), 2.81 (s, 3H), 2.54-2.52 (m, 1H), 2.25 (ddd, J=13.6, 8.1, 5.8 Hz, 1H).

### Example 57: (R)-3-Hydroxy-1-methyl-3-(3-(3-(1-methylimidazo[1,5-a]pyridin-6-yl)phenyl)isoxazol-5-yl)pyrrolidin-2-one

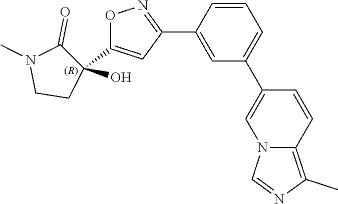

(R)-3-Hydroxy-1-methyl-3-(3-(3-(1-methylimidazo[1,5-a]pyridin-6-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-1-methylimidazo[1,5-a]pyridine in place of 6-chloro-4-methoxy-1-methyl-1H-pyrazolo[3,4-b]pyridine, to give the title compound as a solid (15.6 mg). Purification: RP HPLC; column: SunFire C18 100×19 mm, 5 m. MS (ESI): Mass calcd. for C₂₂H₂₀N₄O₃ 388.15, m/z found, 389.2 [M+H]⁺. ¹H NMR (400 MHz, DMSO-d₆) δ 9.12 (s, 1H), 8.55 (s, 1H), 8.34 (s, 1H), 8.06 (d, J=7.8 Hz, 1H), 7.97 (d, J=8.0 Hz, 1H), 7.72 (t, J=7.7 Hz, 1H), 7.09 (s, 1H), 6.76 (s, 1H), 4.20 (s, 3H), 3.44 (s, 2H), 2.84 (s, 3H), 2.27 (s, 2H).

### Example 58: (R)-3-hydroxy-1-methyl-3-(3-(3-(pyrazolo[1,5-a]pyridin-5-yl)phenyl)isoxazol-5-yl)pyrrolidin-2-one

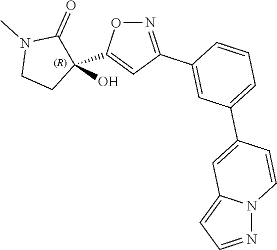

(R)-3-Hydroxy-1-methyl-3-(3-(3-(pyrazolo[1,5-a]pyridin-5-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromopyrazolo[1,5-a]pyridine in place of 6-chloro-4-methoxy-1-methyl-1H-pyrazolo[3,4-b]pyridine, to give the title compound as a solid (20.4 mg). Purification: RP HPLC; column: SunFire C18 100×18 mm, 5 m. MS (ESI): Mass calcd. for C₂₁H₁₈N₄O₃ 374.1, m/z found, 375.4 [M+H]⁺. ¹H NMR (600 MHz, DMSO-d₆) δ 8.75 (d, J=7.3 Hz, 1H), 8.24 (d, J=1.8 Hz, 1H), 8.14 (d, J=2.0 Hz, 1H), 8.02 (d, J=2.3 Hz, 1H), 7.95-7.81 (m, 2H), 7.62 (t, J=7.8 Hz, 1H), 7.33 (dd, J=7.3, 2.1 Hz, 1H), 7.19 (s, 1H), 6.70 (s, 1H), 6.66 (d, J=2.3 Hz, 1H), 3.46 (ddd, J=9.9, 8.0, 4.7 Hz, 1H), 3.43-3.33 (m, 1H), 2.82 (s, 3H), 2.60-2.55 (m, 1H), 2.26 (ddd, J=13.5, 8.0, 5.7 Hz, 1H).

### Example 59: (R)-4-(3-(5-(3-hydroxy-1-methyl-2-oxopyrrolidin-3-yl)isoxazol-3-yl)phenyl)isoindolin-1-one

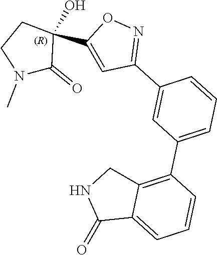

(R)-4-(3-(5-(3-hydroxy-1-methyl-2-oxopyrrolidin-3-yl)isoxazol-3-yl)phenyl)isoindolin-1-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-bromo-2,3-dihydro-1H-isoindol-1-one in place of 6-chloro-4-methoxy-1-methyl-1H-pyrazolo[3,4-b]pyridine, to give the title compound as a solid (12.1 mg). Purification: RP HPLC; column SunFire 100×19 mm, 5 m. MS (ESI): Mass calcd. for C₂₂H₁₉N₃O₄ 389.14, m/z found, 390.4 [M+H]⁺. ¹H NMR (500 MHz, Acetonitrile-d₃) δ 8.01 (s, 1H), 7.93 (d, J=7.6 Hz, 1H), 7.80 (d, J=7.5 Hz, 1H), 7.73 (d, J=7.5 Hz, 1H), 7.71-7.52 (m, 3H), 6.88 (s, 2H), 4.51 (d, J=8.9 Hz, 3H), 3.50 (dd, J=7.6, 5.6 Hz, 2H), 2.90 (s, 3H), 2.67 (dt, J=13.5, 5.7 Hz, 1H), 2.36 (dt, J=14.2, 7.5 Hz, 1H).

### Example 60: (R)-4-(3-(5-(3-Hydroxy-1-methyl-2-oxopyrrolidin-3-yl)isoxazol-3-yl)phenyl)isoindolin-1-one

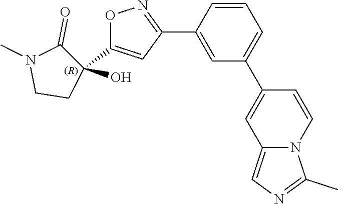

(R)-3-hydroxy-1-methyl-3-(3-(3-(3-methylimidazo[1,5-a]pyridin-7-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 7-bromo-3-methylimidazo[1,5-a]pyridine in place of 6-chloro-4-methoxy-1-methyl-1H-pyrazolo[3,4-b]pyridine, to give the title compound as a solid (20.2 mg). Purification: RP HPLC; column: SUNFIRE C18 100×20 mm, 5 m. MS (ESI): Mass calcd. for C₂₂H₂₀N₄O₃ 388.15, m/z found, 389.0 [M+H]⁺. ¹H NMR (600 MHz, DMSO-d₆) δ 8.18 (d, J=1.8 Hz, 1H), 8.15 (d, J=7.5 Hz, 1H), 7.98 (s, 1H), 7.92-7.77 (m, 2H), 7.57 (t, J=7.8 Hz, 1H), 7.33 (s, 1H), 7.18 (s, 1H), 7.13 (dd, J=7.4, 1.8 Hz, 1H), 6.70 (s, 1H), 3.52-3.44 (m, 1H), 3.44-3.37 (m, 1H), 3.14 (s, 1H), 2.82 (s, 3H), 2.60 (s, 3H), 2.26 (ddd, J=13.6, 8.1, 5.8 Hz, 1H).

### Example 61: (R)-3-Hydroxy-3-(3-(3-(imidazo[1,2-a]pyrazin-3-yl)phenyl)isoxazol-5-yl)-1-methylpyrrolidin-2-one

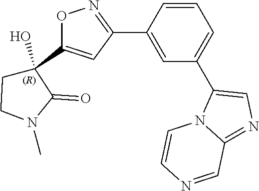

(R)-3-Hydroxy-3-(3-(3-(imidazo[1,2-a]pyrazin-3-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 3-chloroimidazo[1,2-a]pyrazine in place of 6-chloro-4-methoxy-1-methyl-1H-pyrazolo[3,4-b]pyridine, to give the title compound as a solid (17.5 mg). Purification: RP HPLC; column SunFire 100×19 mm, 5 m. MS (ESI): Mass calcd. for C₂₀H₁₇N₅O₃ 375.13, m/z found, 376.0 [M+H]⁺. ¹H NMR (500 MHz, DMSO-d₆) δ 9.15 (s, 1H), 8.66 (d, J=4.8 Hz, 1H), 8.19 (s, 1H), 8.16 (s, 1H), 7.96 (dd, J=14.1, 6.2 Hz, 2H), 7.86 (d, J=7.7 Hz, 1H), 7.71 (t, J=7.8 Hz, 1H), 7.15 (s, 1H), 6.72 (s, 1H), 3.47 (dt, J=8.3, 4.2 Hz, 1H), 3.42 (td, J=14.2, 12.7, 7.2 Hz, 1H), 2.82 (s, 3H), 2.26 (ddd, J=13.6, 8.0, 5.6 Hz, 1H), 2.06 (s, 1H).

### Example 62: (R)-3-Hydroxy-1-methyl-3-(3-(3-(1-methyl-1H-benzo[d]imidazol-6-yl)phenyl)isoxazol-5-yl)pyrrolidin-2-one

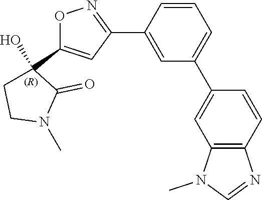

(R)-3-Hydroxy-1-methyl-3-(3-(3-(1-methyl-1H-benzo[d]imidazol-6-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-1-methyl-1H-1,3-benzodiazole in place of 6-chloro-4-methoxy-1-methyl-1H-pyrazolo[3,4-b]pyridine, to give the title compound as a solid (12.1 mg). Purification: RP HPLC; column SunFire 100×19 mm, 5 m. MS (ESI): Mass calcd. for C₂₂H₂₀N₄O₃ 388.2, m/z found, 389.2 [M+H]⁺. ¹H NMR (500 MHz, DMSO-d₆) δ 8.22 (s, 1H), 8.21-8.11 (m, 1H), 7.96 (d, J=1.7 Hz, 1H), 7.85 (dd, J=11.8, 7.8 Hz, 2H), 7.72 (d, J=8.4 Hz, 1H), 7.60 (ddd, J=7.8, 4.4, 2.7 Hz, 2H), 7.14 (s, 1H), 6.73 (s, 1H), 3.90 (s, 3H), 3.47 (dt, J=9.9, 4.1 Hz, 1H), 3.45-3.37 (m, 1H), 3.15 (d, J=4.5 Hz, 1H), 2.83 (s, 3H), 2.27 (ddd, J=13.6, 8.1, 5.7 Hz, 1H).

### Example 63: (R)-3-Hydroxy-1-methyl-3-(3-(3-(2-methylbenzo[d]oxazol-5-yl)phenyl)isoxazol-5-yl)pyrrolidin-2-one

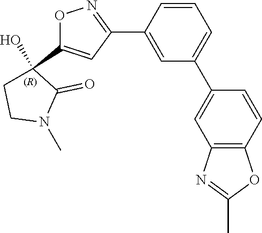

(R)-3-Hydroxy-1-methyl-3-(3-(3-(2-methylbenzo[d]oxazol-5-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-chloro-2-methyl-1,3-benzoxazole in place of 6-chloro-4-methoxy-1-methyl-1H-pyrazolo[3,4-b]pyridine, to give the title compound as a solid (19.0 mg). Purification: RP HPLC; column SunFire C18 100×19 mm, 5 m. MS (ESI): Mass calcd. for C₂₂H₁₉N₃O₄ 389.1, m/z found, 390.2 [M+H]⁺. ¹H NMR (600 MHz, DMSO-d₆) δ 8.15 (d, J=1.8 Hz, 1H), 8.03 (d, J=1.6 Hz, 1H), 7.85 (d, J=7.7 Hz, 1H), 7.82 (d, J=8.0 Hz, 1H), 7.77-7.68 (m, 2H), 7.58 (t, J=7.8 Hz, 1H), 7.17 (s, 1H), 6.68 (s, 1H), 3.45 (ddd, J=9.9, 8.1, 4.7 Hz, 1H), 3.41 (dd, J=7.6, 5.9 Hz, 1H), 2.81 (s, 3H), 2.62 (s, 3H), 2.54 (d, J=5.1 Hz, 1H), 2.25 (ddd, J=13.7, 8.1, 5.9 Hz, 1H).

### Example 64: (R)-3-Hydroxy-1-methyl-3-(3-(3-(pyrrolo[1,2-b]pyridazin-5-yl)phenyl)isoxazol-5-yl)pyrrolidin-2-one

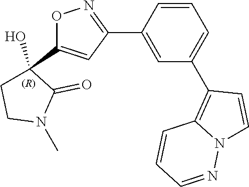

(R)-3-Hydroxy-1-methyl-3-(3-(3-(pyrrolo[1,2-b]pyridazin-5-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromopyrrolo[1,2-b]pyridazine in place of 6-chloro-4-methoxy-1-methyl-1H-pyrazolo[3,4-b]pyridine, to give the title compound as a solid (12.3 mg). Purification: RP HPLC; column: SUNFIRE C18 100×20 mm, 5 m. MS (ESI): Mass calcd. for C₂₆H₂₆N₆O 438.2, m/z found, 439.2 [M+H]⁺. ¹H NMR (600 MHz, DMSO-d₆) δ 14.12 (s, 1H), 8.54-8.45 (m, 1H), 8.38 (s, 1H), 8.22 (s, 1H), 8.06 (s, 1H), 7.66 (dd, J=11.6, 7.5 Hz, 3H), 7.59 (dd, J=9.0, 3.1 Hz, 1H), 7.45 (q, J=8.0 Hz, 3H), 7.40-7.30 (m, 3H), 3.35 (t, J=5.1 Hz, 4H), 2.51 (s, 4H), 2.23 (s, 3H).

### Example 65: (R)-3-Hydroxy-1-methyl-3-(3-(3-(2-methylimidazo[1,2-b]pyridazin-6-yl)phenyl)isoxazol-5-yl)pyrrolidin-2-one

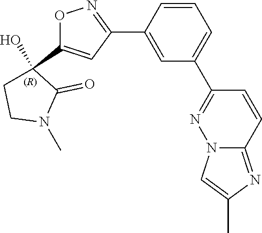

(R)-3-Hydroxy-1-methyl-3-(3-(3-(2-methylimidazo[1,2-b]pyridazin-6-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-2-methylimidazo[1,2-b]pyridazine hydrobromide in place of 6-chloro-4-methoxy-1-methyl-1H-pyrazolo[3,4-b]pyridine, to give the title compound as a solid (31.7 mg). Purification: RP HPLC; column SunFire C18 100×19 mm, 5 m. MS (ESI): Mass calcd. for C₂₁H₁₉N₅O₃ 389.2, m/z found, 390.4 [M+H]⁺. ¹H NMR (500 MHz, Acetonitrile-d₃) δ 8.46 (d, J=2.0 Hz, 1H), 8.13 (d, J=8.0 Hz, 1H), 7.98 (dd, J=18.5, 8.6 Hz, 2H), 7.92 (s, 1H), 7.77-7.58 (m, 2H), 6.93 (s, 1H), 4.49 (s, 1H), 3.51 (dd, J=7.6, 5.6 Hz, 2H), 2.91 (s, 3H), 2.69 (dt, J=13.7, 5.5 Hz, 1H), 2.48 (s, 3H), 2.37 (dd, J=13.7, 7.2 Hz, 1H).

### Example 66: (R)-3-(3-(3-(Furo[3,2-b]pyridin-5-yl)phenyl)isoxazol-5-yl)-3-hydroxy-1-methylpyrrolidin-2-one

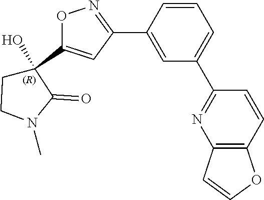

(R)-3-(3-(3-(Furo[3,2-b]pyridin-5-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromofuro[3,2-b]pyridine in place of 6-chloro-4-methoxy-1-methyl-1H-pyrazolo[3,4-b]pyridine, to give the title compound as a solid (12.3 mg). Purification: RP HPLC; column: SunFire C18 100×19 mm, 5 m. MS (ESI): Mass calcd. for C₂₁H₁₇N₃O₄ 375.1, m/z found, 376.2 [M+H]⁺. ¹H NMR (500 MHz, Acetonitrile-d₃) δ 8.55 (d, J=2.0 Hz, 1H), 8.21 (d, J=7.9 Hz, 1H), 8.09 (d, J=2.4 Hz, 1H), 7.99 (d, J=8.7 Hz, 1H), 7.96-7.78 (m, 2H), 7.64 (t, J=7.8 Hz, 1H), 7.09 (d, J=2.4 Hz, 1H), 6.92 (s, 1H), 4.54 (s, 1H), 3.51 (dd, J=7.6, 5.6 Hz, 2H), 2.91 (s, 3H), 2.69 (dt, J=13.5, 5.6 Hz, 1H), 2.38 (dt, J=13.4, 7.5 Hz, 1H).

### Example 67: (R)-3-Hydroxy-3-(3-(3-(imidazo[1,5-a]pyridin-7-yl)phenyl)isoxazol-5-yl)-1-methylpyrrolidin-2-one

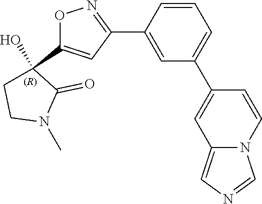

(R)-3-hydroxy-3-(3-(3-(imidazo[1,5-a]pyridin-7-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 7-bromoimidazo[1,5-a]pyridine in place of 6-chloro-4-methoxy-1-methyl-1H-pyrazolo[3,4-b]pyridine, to give the title compound as a solid (23.6 mg). Purification: RP HPLC; column SunFire C18 100×19 mm, 5 m. MS (ESI): Mass calcd. for C₂₁H₁₈N₄O₃ 374.1, m/z found, 375.4 [M+H]⁺. ¹H NMR (500 MHz, Acetonitrile-d) δ 8.33-8.20 (m, 1H), 8.15 (d, J=1.9 Hz, 1H), 7.88 (d, J=6.5 Hz, 1H), 7.82 (d, J=7.8 Hz, 1H), 7.61 (t, J=7.8 Hz, 1H), 7.45 (s, 1H), 7.04 (dd, J=7.5, 1.9 Hz, 1H), 6.92 (s, 1H), 4.53 (s, 1H), 3.50 (dd, J=7.6, 5.6 Hz, 2H), 2.92 (s, 3H), 2.82-2.60 (m, 2H), 2.37 (dt, J=13.4, 7.5 Hz, 2H).

### Example 68: (R)-3-Hydroxy-1-methyl-3-(3-(3-(pyrazolo[1,5-a]pyrimidin-5-yl)phenyl)isoxazol-5-yl)pyrrolidin-2-one

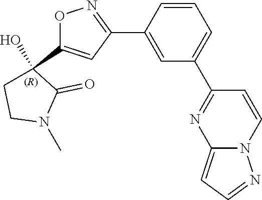

(R)-3-Hydroxy-1-methyl-3-(3-(3-(pyrazolo[1,5-a]pyrimidin-5-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromopyrazolo[1,5-a]pyrimidine in place of 6-chloro-4-methoxy-1-methyl-1H-pyrazolo[3,4-b]pyridine, to give the title compound as a solid (18.5 mg). Purification: RP HPLC; column: SUNFIRE C18 100×20 mm, 5 m. MS (ESI): Mass calcd. for C₂₀H₁₇N₅O₃ 375.1, m/z found, 376.4 [M+H]⁺. H NMR (500 MHz, Acetonitrile-d₃) δ 8.88 (d, J=7.4 Hz, 1H), 8.60 (d, J=2.0 Hz, 1H), 8.28 (d, J=7.9 Hz, 1H), 8.17 (d, J=2.4 Hz, 1H), 8.00 (d, J=7.7 Hz, 1H), 7.68 (t, J=7.8 Hz, 1H), 7.55 (d, J=7.4 Hz, 1H), 6.94 (s, 1H), 6.72 (d, J=2.3 Hz, 1H), 4.64 (s, 1H), 3.51 (dd, J=7.6, 5.6 Hz, 2H), 2.91 (s, 3H), 2.69 (dt, J=13.6, 5.7 Hz, 1H), 2.39 (dt, J=14.1, 7.5 Hz, 1H).

### Example 69: (R)-3-(3-(3-(Benzo[d]oxazol-6-yl)phenyl)isoxazol-5-yl)-3-hydroxy-1-methylpyrrolidin-2-one

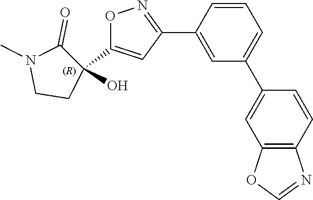

(R)-3-(3-(3-(Benzo[d]oxazol-6-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-1,3-benzoxazole in place of 6-chloro-4-methoxy-1-methyl-1H-pyrazolo[3,4-b]pyridine, to give the title compound as a solid (18.8 mg). Purification: RP HPLC; column SunFire 100×19 mm, 5 m. MS (ESI): Mass calcd. for C₂₁H₁₇N₃O₄ 375.1, m/z found, 376.2 [M+H]⁺. ¹H NMR (500 MHz, DMSO-d₆) δ 8.79 (s, 1H), 8.21 (d, J=8.2 Hz, 2H), 7.89 (d, J=7.9 Hz, 2H), 7.81 (d, J=8.3 Hz, 1H), 7.61 (t, J=7.7 Hz, 1H), 7.19 (s, 1H), 6.70 (s, 1H), 3.46 (td, J=9.3, 5.0 Hz, 1H), 3.42 (q, J=4.9, 3.0 Hz, 1H), 2.83 (s, 3H), 2.56 (d, J=6.9 Hz, 1H), 2.52 (s, 1H), 2.26 (dt, J=13.8, 6.9 Hz, 1H).

### Example 70: (R)-3-Hydroxy-3-(3-(3-(imidazo[1,2-a]pyrazin-6-yl)phenyl)isoxazol-5-yl)-1-methylpyrrolidin-2-one

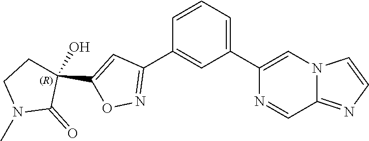

(R)-3-Hydroxy-3-(3-(3-(imidazo[1,2-a]pyrazin-6-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-chloroimidazo[1,2-a]pyrazine in place of 6-chloro-4-methoxy-1-methyl-1H-pyrazolo[3,4-b]pyridine, to give the title compound as a solid (15.1 mg). MS (ESI): Mass calcd. for C₂₀H₁₇N₅O₃ 375.1, m/z found, 376.0 [M+H]⁺. ¹H NMR (600 MHz, Acetonitrile-d₃) δ 9.09 (d, J=1.5 Hz, 1H), 8.93 (d, J=1.6 Hz, 1H), 8.47 (t, J=1.8 Hz, 1H), 8.15 (d, J=7.7 Hz, 1H), 7.96 (s, 1H), 7.90 (d, J=7.7 Hz, 1H), 7.80 (s, 1H), 7.64 (t, J=7.8 Hz, 1H), 6.91 (s, 1H), 4.47 (s, 1H), 3.50 (dd, J=7.5, 5.7 Hz, 2H), 2.90 (s, 3H), 2.69 (dt, J=13.5, 5.6 Hz, 1H), 2.37 (dt, J=14.1, 7.6 Hz, 1H).

### Example 71: (R)-3-Hydroxy-3-(3-(3-(imidazo[1,2-a]pyridin-6-yl)phenyl)isoxazol-5-yl)-1-methylpyrrolidin-2-one

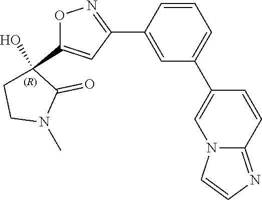

(R)-3-Hydroxy-3-(3-(3-(imidazo[1,2-a]pyridin-6-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromoimidazo[1,2-a]pyridine in place of 6-chloro-4-methoxy-1-methyl-1H-pyrazolo[3,4-b]pyridine, to give the title compound as a solid (12.0 mg). Purification: RP HPLC; column SunFire C18 100×19 mm, 5 m. MS (ESI): Mass calcd. for C₂₁H₁₈N₄O₃ 374.1, m/z found, 375.2 [M+H]⁺. ¹H NMR (500 MHz, Acetonitrile-d₃) δ 8.70 (s, 1H), 8.12 (d, J=1.9 Hz, 1H), 7.90 (d, J=7.7 Hz, 1H), 7.83 (s, 1H), 7.79 (d, J=7.6 Hz, 1H), 7.70-7.53 (m, 3H), 6.92 (s, 1H), 4.62 (s, 1H), 3.50 (dd, J=7.6, 5.6 Hz, 2H), 2.69 (dt, J=13.5, 5.7 Hz, 1H), 2.52 (s, 3H), 2.38 (dt, J=13.4, 7.5 Hz, 1H).

### Example 72: (R)-3-Hydroxy-1-methyl-3-(3-(3-(thieno[2,3-d]pyrimidin-4-yl)phenyl)isoxazol-5-yl)pyrrolidin-2-one

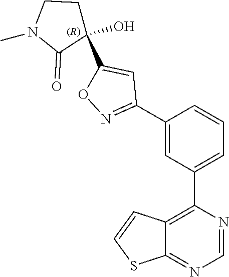

(R)-3-Hydroxy-1-methyl-3-(3-(3-(thieno[2,3-d]pyrimidin-4-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-chlorothieno[2,3-d]pyrimidine in place of 6-chloro-4-methoxy-1-methyl-1H-pyrazolo[3,4-b]pyridine, to give the title compound as a solid (18.0 mg). Purification: RP HPLC; column SunFire C18 100×19 mm, 5 m. MS (ESI): Mass calcd. for C₂₀H₁₆N₄O₃S 392.1, m/z found, 393.2 [M+H]⁺. ¹H NMR (500 MHz, Acetonitrile-d₃) δ 9.15 (s, 1H), 8.45 (s, 1H), 8.10 (dd, J=19.9, 7.7 Hz, 2H), 7.83 (d, J=6.1 Hz, 1H), 7.78-7.51 (m, 2H), 6.91 (s, 1H), 4.50 (s, 1H), 3.64-3.43 (m, 2H), 3.04 (s, 3H), 2.69 (dt, J=12.0, 5.8 Hz, 1H), 2.37 (dt, J=14.2, 7.6 Hz, 1H).

### Example 73: (R)-3-(3-(3-(5-Ethyl-7H-pyrrolo[2,3-d]pyrimidin-4-yl)phenyl)isoxazol-5-yl)-3-hydroxy-1-methylpyrrolidin-2-one

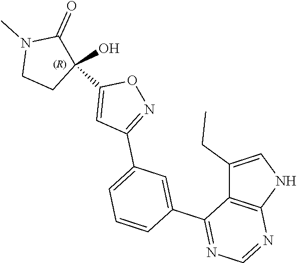

(R)-3-(3-(3-(5-Ethyl-7H-pyrrolo[2,3-d]pyrimidin-4-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-chloro-5-ethyl-7H-pyrrolo[2,3-d]pyrimidine in place of 6-chloro-4-methoxy-1-methyl-1H-pyrazolo[3,4-b]pyridine, to give the title compound as a solid (4.0 mg). Purification: RP HPLC; column SunFireC18 100×19 mm, 5 m. MS (ESI): Mass calcd. for C₂₂H₂₁N₅O₃ 403.2, m/z found, 404.2 [M+H]⁺. ¹H NMR (500 MHz, Acetonitrile-d₃) δ 9.15 (s, 1H), 8.45 (s, 1H), 8.10 (dd, J=19.9, 7.7 Hz, 2H), 7.83 (d, J=6.1 Hz, 1H), 7.78-7.51 (m, 2H), 6.91 (s, 1H), 4.50 (s, 1H), 3.64-3.43 (m, 2H), 3.04 (s, 3H), 2.69 (dt, J=12.0, 5.8 Hz, 1H), 2.37 (dt, J=14.2, 7.6 Hz, 1H).

### Example 74: (R)-3-(3-(3-(3-Amino-[1,2,4]triazolo[4,3-a]pyridin-6-yl)phenyl)isoxazol-5-yl)-3-hydroxy-1-methylpyrrolidin-2-one

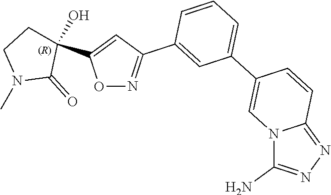

(R)-3-(3-(3-(3-Amino-[1,2,4]triazolo[4,3-a]pyridin-6-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-[1,2,4]triazolo[4,3-a]pyridin-3-amine in place of 6-chloro-4-methoxy-1-methyl-1H-pyrazolo[3,4-b]pyridine, to give the title compound as a solid (31.3 mg). Purification: RP HPLC; column: SunFire C18 100×18 mm, 5 m). MS (ESI): Mass calcd. for C₂₀H₁₈N₆O₃ 390.1 m/z found, 391.4 [M+H]⁺. ¹H NMR (600 MHz, DMSO-d₆) δ 8.50 (d, J=1.5 Hz, 1H), 8.14 (t, J=1.8 Hz, 1H), 7.89 (s, 1H), 7.85-7.74 (m, 1H), 7.62 (t, J=7.8 Hz, 1H), 7.53 (d, J=1.3 Hz, 2H), 7.12 (s, 1H), 6.46 (s, 2H), 3.53-3.35 (m, 2H), 2.82 (s, 3H), 2.59-2.52 (m, 2H), 2.25 (ddd, J=13.6, 8.1, 5.7 Hz, 1H).

### Example 75: (R)-3-(3-(3-(2H-pyrazolo[3,4-c]pyridin-5-yl)phenyl)isoxazol-5-yl)-3-hydroxy-1-methylpyrrolidin-2-one

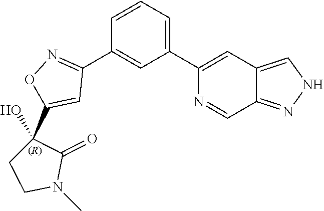

(R)-3-(3-(3-(2H-pyrazolo[3,4-c]pyridin-5-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromo-1H-pyrazolo[3,4-c]pyridine hydrochloride in place of 6-chloro-4-methoxy-1-methyl-1H-pyrazolo[3,4-b]pyridine, to give the title compound as a solid (2.0 mg). MS (ESI): Mass calcd. for C₂₀H₁₇N₅O₃ 375.1, m/z found, 376.2 [M+H]⁺. ¹H NMR (500 MHz, DMSO-d₆) δ 9.15 (s, 1H), 8.59 (s, 1H), 8.45 (s, 1H), 8.27 (s, 1H), 8.24 (d, J=7.9 Hz, 1H), 7.86 (d, J=7.6 Hz, 1H), 7.60 (t, J=7.8 Hz, 1H), 7.11 (s, 1H), 6.64 (d, J=64.6 Hz, 1H), 3.56-3.37 (m, 2H), 2.83 (s, 3H), 2.64-2.52 (m, 1H), 2.27 (ddd, J=13.6, 8.0, 5.8 Hz, 1H), 1.09 (s, 1H).

### Example 76: (R)-3-Hydroxy-3-(3-(3-(4-methoxy-1-methyl-1H-indazol-6-yl)phenyl)isoxazol-5-yl)-1-methylpyrrolidin-2-one

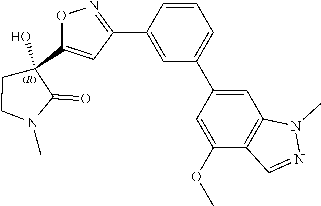

Step A: 6-Bromo-4-methoxy-1-methyl-1H-indazole. To a solution of 6-bromo-4-methoxy-1H-indazole (100 mg, 0.44 mmol) dissolved in DMF/THF (1:1 mixture of 5 mL) was added NaH (53.0 mg, 1.32 mmol, 60% dispersion in mineral oil). After 5 minutes methyl iodide (83 μL, 1.32 mmol) was added and the reaction mixture was heated at 46° C. for 18 hours. The reaction mixture was quenched with NH₄Cl (aq), and the aqueous layer was extracted with EtOAc (3×). The combined organic extracts were washed with brine, dried with MgSO₄, filtered, concentrated and purified with HPLC (Column TRIART 20×100 mm, 5 μm; H₂O/AcCN+NH₃; gradient 40-60%) to provide the title compound (28 mg, 27%). MS (ESI): Mass calcd. for C₉H₉BrN₂O 240.0, m/z found, 241.0 [M+H]⁺.

Step B: (R)-3-Hydroxy-3-(3-(3-(4-methoxy-1-methyl-1H-indazol-6-yl)phenyl)isoxazol-5-yl)-1-methylpyrrolidin-2-one. (R)-3-Hydroxy-3-(3-(3-(4-methoxy-1-methyl-1H-indazol-6-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-4-methoxy-1-methyl-1H-indazole in place of 6-chloro-4-methoxy-1-methyl-1H-pyrazolo[3,4-b]pyridine, to give the title compound as a solid (2.4 mg). MS (ESI): Mass calcd. for C₂₃H₂₂N₄O₄ 418.5, m/z found, 419.4 [M+H]⁺. ¹H NMR (600 MHz, Acetonitrile-d₃) δ 8.18 (t, J=1.8 Hz, 1H), 7.97 (d, J=0.9 Hz, 1H), 7.90-7.84 (m, 2H), 7.60 (t, J=7.7 Hz, 1H), 7.40 (d, J=1.0 Hz, 1H), 6.94 (s, 1H), 6.86 (d, J=1.0 Hz, 1H), 4.61 (s, 1H), 4.06 (d, J=10.3 Hz, 6H), 3.52-3.46 (m, 2H), 2.90 (s, 3H), 2.71-2.64 (m, 1H), 2.41-2.32 (m, 1H).

### Example 77: (R)-3-(3-(3-(1H-Indol-4-yl)phenyl)isoxazol-5-yl)-3-hydroxy-1-methylpyrrolidin-2-one

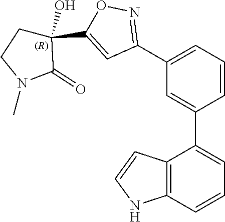

(R)-3-(3-(3-(1H-indol-4-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 4-bromoindole in place of 6-chloro-4-methoxy-1-methyl-1H-pyrazolo[3,4-b]pyridine, to give the title compound as a solid (3.0 mg). MS (ESI): Mass calcd. for C₂₂H₁₉N₃O₃ 373.4, m/z found, 374.2 [M+H]⁺. ¹H NMR (400 MHz, DMSO-d₆) δ 11.32 (s, 1H), 8.13 (s, 1H), 7.86 (s, 1H), 7.79 (s, 1H), 7.64 (s, 1H), 7.44 (s, 2H), 7.20 (d, J=12.7 Hz, 2H), 7.08 (s, 1H), 6.72 (s, 1H), 6.56 (s, 1H), 3.45 (s, 2H), 2.84 (s, 3H), 2.36-2.25 (m, 2H).

### Example 78: (R)-3-Hydroxy-1-methyl-3-(3-(3-(1-methyl-1H-pyrazolo[3,4-b]pyridin-6-yl)phenyl)isoxazol-5-yl)pyrrolidin-2-one

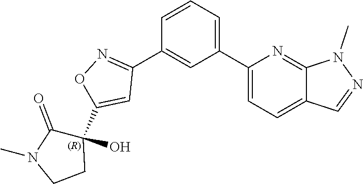

(R)-3-Hydroxy-1-methyl-3-(3-(3-(1-methyl-1H-pyrazolo[3,4-b]pyridin-6-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-chloro-1-methyl-1H-pyrazolo[3,4-B]pyridine in place of 6-chloro-4-methoxy-1-methyl-1H-pyrazolo[3,4-b]pyridine, to give the title compound as a solid (3.2 mg). MS (ESI): Mass calcd. for C₂₁H₁₉N₅O₃ 389.4, m/z found, 390.4 [M+H]⁺. ¹H NMR (500 MHz, DMSO-d₆) δ 8.64 (t, J=1.8 Hz, 1H), 8.37-8.31 (m, 2H), 8.16 (d, J=1.5 Hz, 1H), 8.00-7.91 (m, 2H), 7.71-7.63 (m, 1H), 7.16 (d, J=1.6 Hz, 1H), 6.72 (s, 1H), 4.13 (d, J=1.5 Hz, 3H), 3.51-3.38 (m, 2H), 2.84 (s, 1H), 2.83 (s, 2H), 2.62-2.50 (m, 1H), 2.32-2.23 (m, 1H).

### Example 79: (R)-3-Hydroxy-1-methyl-3-(3-(3-(2-methylimidazo[1,2-a]pyridin-6-yl)phenyl)isoxazol-5-yl)pyrrolidin-2-one

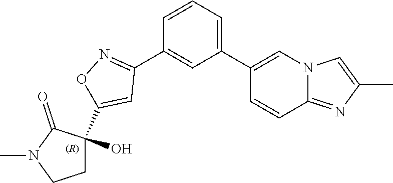

(R)-3-Hydroxy-1-methyl-3-(3-(3-(2-methylimidazo[1,2-a]pyridin-6-yl)phenyl)isoxazol-5-yl)pyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-bromo-2-methylimidazo[1,2-a]pyridine in place of 6-chloro-4-methoxy-1-methyl-1H-pyrazolo[3,4-b]pyridine, to give the title compound as a solid (5.3 mg). MS (ESI): Mass calcd. for C₂₂H₂₀N₄O₃ 388.4, m/z found, 389.2 [M+H]⁺. ¹H NMR (500 MHz, Acetonitrile-d₃) δ 8.61 (s, 1H), 8.10 (d, J=2.1 Hz, 1H), 7.88 (d, J=7.7 Hz, 1H), 7.77 (d, J=7.8 Hz, 1H), 7.65-7.50 (m, 4H), 6.91 (s, 1H), 3.54-3.47 (m, 2H), 2.91 (s, 3H), 2.73-2.64 (m, 1H), 2.44-2.33 (m, 4H), 1.95-1.90 (m, 1H).

### Example 80: (R)-7-(3-(5-(3-Hydroxy-1-methyl-2-oxopyrrolidin-3-yl)isoxazol-3-yl)phenyl)-1-methyl-3,4-dihydroquinoxalin-2(1H)-one

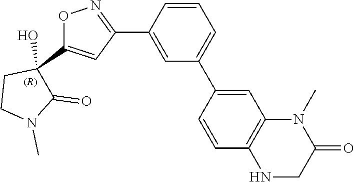

(R)-7-(3-(5-(3-Hydroxy-1-methyl-2-oxopyrrolidin-3-yl)isoxazol-3-yl)phenyl)-1-methyl-3,4-dihydroquinoxalin-2(1H)-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 7-bromo-1-methyl-1,2,3,4-tetrahydroquinoxalin-2-one in place of 6-chloro-4-methoxy-1-methyl-1H-pyrazolo[3,4-b]pyridine, to give the title compound as a solid (6.1 mg). MS (ESI): Mass calcd. for C₂₃H₂₂N₄O₄ 418.5, m/z found, 419.2 [M+H]⁺. ¹H NMR (600 MHz, Acetonitrile-d₃) δ 8.03 (d, J=2.0 Hz, 1H), 7.81-7.74 (m, 1H), 7.74-7.69 (m, 1H), 7.53 (t, J=7.7 Hz, 1H), 7.28 (d, J=1.9 Hz, 1H), 7.27-7.23 (m, 1H), 6.90 (s, 1H), 6.83 (d, J=8.1 Hz, 1H), 4.84 (s, 1H), 3.87 (d, J=1.8 Hz, 2H), 3.71 (s, 1H), 3.53-3.41 (m, 2H), 3.38 (d, J=3.6 Hz, 3H), 2.89 (d, J=3.3 Hz, 3H), 2.74-2.59 (m, 1H), 2.41-2.31 (m, 1H).

### Example 81: (R)-3-Hydroxy-3-(3-(3-(6-methoxy-1,3-dihydroisobenzofuran-5-yl)phenyl)isoxazol-5-yl)-1-methylpyrrolidin-2-one

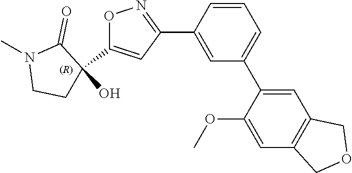

(R)-3-Hydroxy-3-(3-(3-(6-methoxy-1,3-dihydroisobenzofuran-5-yl)phenyl)isoxazol-5-yl)-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromo-6-methoxy-1,3-dihydro-2-benzofuran in place of 6-chloro-4-methoxy-1-methyl-1H-pyrazolo[3,4-b]pyridine, to give the title compound as a solid (10.2 mg). MS (ESI): Mass calcd. for C₂₃H₂₂N₂O₅ 406.4, m/z found, 407.2 [M+H]⁺. ¹H NMR (500 MHz, Acetonitrile-d₃) δ 7.96 (d, J=1.9 Hz, 1H), 7.82 (d, J=7.9 Hz, 1H), 7.63 (d, J=7.8 Hz, 1H), 7.54 (t, J=7.7 Hz, 1H), 7.28 (s, 1H), 7.06 (s, 1H), 6.84 (s, 1H), 5.13-4.98 (m, 4H), 4.51 (s, 1H), 3.81 (s, 3H), 3.53-3.41 (m, 2H), 2.90 (s, 3H), 2.72-2.60 (m, 1H), 2.41-2.30 (m, 1H).

### Example 82: (R)-5-(3-(5-(3-Hydroxy-1-methyl-2-oxopyrrolidin-3-yl)isoxazol-3-yl)phenyl)isoquinolin-1 (2H)-one

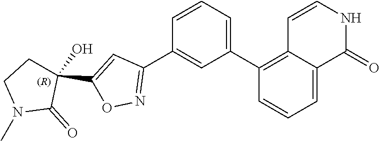

(R)-5-(3-(5-(3-Hydroxy-1-methyl-2-oxopyrrolidin-3-yl)isoxazol-3-yl)phenyl)isoquinolin-1 (2H)-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 5-bromo-1,2-dihydroisoquinolin-1-one in place of 6-chloro-4-methoxy-1-methyl-1H-pyrazolo[3,4-b]pyridine, to give the title compound as a solid (4.0 mg). MS (ESI): Mass calcd. for C₂₃H₁₉N₃O₄ 401.4, m/z found, 402.2 [M+H]⁺. ¹H NMR (600 MHz, DMSO-d₆) δ 11.35 (s, 1H), 8.25 (d, J=8.0 Hz, 1H), 7.94 (d, J=7.8 Hz, 1H), 7.89 (d, J=1.8 Hz, 1H), 7.70-7.66 (m, 1H), 7.63 (t, J=7.7 Hz, 1H), 7.58-7.51 (m, 2H), 7.14 (d, J=7.4 Hz, 1H), 7.07 (s, 1H), 6.32 (d, J=7.4 Hz, 1H), 3.46-3.41 (m, 1H), 3.41-3.35 (m, 1H), 3.14 (s, 1H), 2.80 (s, 4H), 2.62-2.57 (m, 1H).

### Example 83: (R)-3-(3-(3-(1H-pyrazolo[4,3-c]pyridin-6-yl)phenyl)isoxazol-5-yl)-3-hydroxy-1-methylpyrrolidin-2-one

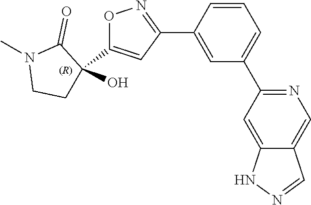

(R)-3-(3-(3-(1H-pyrazolo[4,3-c]pyridin-6-yl)phenyl)isoxazol-5-yl)-3-hydroxy-1-methylpyrrolidin-2-one was synthesized in a manner analogous to Example 48, (R)-3-hydroxy-3-(3-(3-(4-methoxy-1-methyl-1H-pyrazolo[3,4-b]pyridin-6-yl)phenyl)isoxazol-5-yl)-1-methylpyrrolidin-2-one, using 6-chloro-1H-pyrazolo[4,3-c]pyridine in place of 6-chloro-4-methoxy-1-methyl-1H-pyrazolo[3,4-b]pyridine, to give the title compound as a solid (1.7 mg). MS (ESI): Mass calcd. for C₂₀H₁₇N₅O₃ 375.4 m/z found, 376.2 [M+H]⁺. ¹H NMR (500 MHz, DMSO-d₆) δ 13.54 (s, 1H), 9.21 (s, 1H), 8.63 (s, 1H), 8.33 (s, 1H), 8.28 (d, J=7.8 Hz, 1H), 8.14 (s, 1H), 7.91 (d, J=7.7 Hz, 1H), 7.62 (t, J=7.8 Hz, 1H), 7.12 (s, 1H), 6.65 (d, J=55.7 Hz, 1H), 3.49-3.39 (m, 2H), 2.83 (s, 3H), 2.65-2.53 (m, 1H), 2.32-2.23 (m, 1H).

### Example 84: (R)-6-(3-(5-(3-Hydroxy-1-methyl-2-oxopyrrolidin-3-yl)isoxazol-3-yl)phenyl)-3-methyl-1H-pyrazolo[3,4-b]pyridine-4-carboxamide

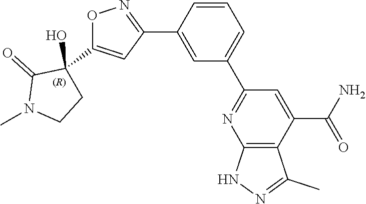

(R)-6-(3-(5-(3-Hydroxy-1-methyl-2-oxopyrrolidin-3-yl)isoxazol-3-yl)phenyl)-3-methyl-1H-pyrazolo[3,4-b]pyridine-4-carboxamide (27 mg) was prepared in a manner analogous to Example 47 using ethyl 6-chloro-3-methyl-1H-pyrazolo[3,4-b]pyridine-4-carboxylate instead of methyl 4-chloro-6-methoxypicolinate in Step A. MS (ESI): Mass calcd. for C₂₂H₂₀N₆O₄, 432.2; m/z found, 433.2 [M+H]⁺. ¹H NMR (600 MHz, CD₃OD) δ 8.70-8.59 (m, 1H), 8.30 (d, J=7.5 Hz, 1H), 7.98 (d, J=6.2 Hz, 1H), 7.89 (s, 1H), 7.69 (t, J=7.8 Hz, 1H), 7.03 (s, 1H), 3.66-3.56 (m, 2H), 3.00 (s, 3H), 2.83-2.71 (m, 1H), 2.64 (s, 3H), 2.49-2.40 (m, 1H).

### Example 85: (R)-4-Cyclopropyl-6-(3-(5-(3-hydroxy-1-methyl-2-oxopyrrolidin-3-yl)isoxazol-3-yl)phenyl)pyrimidine-2-carboxamide

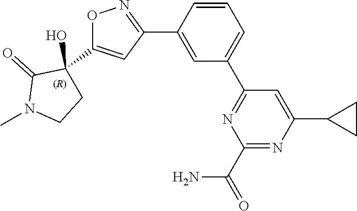

(R)-4-Cyclopropyl-6-(3-(5-(3-hydroxy-1-methyl-2-oxopyrrolidin-3-yl)isoxazol-3-yl)phenyl)pyrimidine-2-carboxamide (57 mg) was prepared in a manner analogous to Example 47 using methyl 4-chloro-6-cyclopropylpyrimidine-2-carboxylate instead of methyl 4-chloro-6-methoxypicolinate in Step A. MS (ESI): Mass calcd. for C₂₂H₂₁N₅O₄, 419.2; m/z found, 420.2 [M+H]⁺. ¹H NMR (500 MHz, CD₃OD) δ 8.79 (t, J=1.8 Hz, 1H), 8.49-8.35 (m, 1H), 8.12-8.02 (m, 2H), 7.67 (t, J=7.8 Hz, 1H), 7.11 (s, 1H), 3.63-3.52 (m, 2H), 2.97 (s, 3H), 2.82-2.69 (m, 1H), 2.47-2.36 (m, 1H), 2.36-2.26 (m, 1H), 1.41-1.29 (m, 2H), 1.25-1.14 (m, 2H).

### Example 86: (R)-3-(3-(3-(2-Aminoquinazolin-8-yl)phenyl)isoxazol-5-yl)-3-hydroxy-1-methylpyrrolidin-2-one

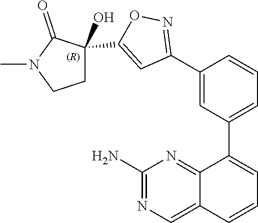

(R)-3-(3-(3-(2-Aminoquinazolin-8-yl)phenyl)isoxazol-5-yl)-3-hydroxy-1-methylpyrrolidin-2-one (88 mg) was prepared in a manner analogous to Example 47 Step B using 8-bromoquinazoline-2-amine instead of 4-chloro-6-methoxypicolinamide. MS (ESI): Mass calcd. for C₂₂H₁₉N₅O₃, 401.1; m/z found, 402.1 [M+H]⁺. ¹H NMR (500 MHz, CD₃OD) δ 8.01-7.94 (m, 1H), 7.89 (t, J=1.8 Hz, 1H), 7.71-7.63 (m, 1H), 7.58-7.51 (m, 1H), 7.49 (dd, J=7.5, 1.6 Hz, 1H), 7.42 (dd, J=7.6, 1.6 Hz, 1H), 7.36 (t, J=7.6 Hz, 1H), 7.01-6.91 (m, 1H), 5.84 (s, 1H), 3.66-3.52 (m, 2H), 2.95 (s, 3H), 2.79-2.68 (m, 1H), 2.44-2.35 (m, 1H).

### Example 87: (R)-6-(3-(5-(3-Hydroxy-1-methyl-2-oxopyrrolidin-3-yl)isoxazol-3-yl)phenyl)-3-methoxypicolinamide

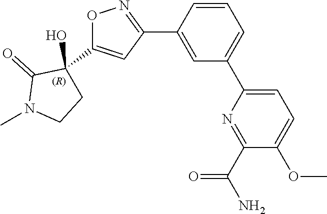

(R)-6-(3-(5-(3-Hydroxy-1-methyl-2-oxopyrrolidin-3-yl)isoxazol-3-yl)phenyl)-3-methoxypicolinamide (125 mg) was prepared in a manner analogous to Example 47 using methyl 6-chloro-3-methoxypyridine-2-carboxylate instead of methyl 4-chloro-6-methoxypicolinate in Step A. MS (ESI): Mass calcd. for C₂₁H₂₀N₄O₅, 408.1; m/z found, 409.1 [M+H]⁺. ¹H NMR (500 MHz, CD₃OD) δ 8.51 (t, J=1.7 Hz, 1H), 8.19-8.10 (m, 1H), 8.06 (d, J=8.8 Hz, 1H), 7.90-7.81 (m, 1H), 7.69 (d, J=8.9 Hz, 1H), 7.61-7.51 (m, 1H), 7.01 (s, 1H), 3.97 (s, 3H), 3.64-3.50 (m, 2H), 2.96 (s, 3H), 2.79-2.68 (m, 1H), 2.46-2.34 (m, 1H).

### Example 88: (R)-3-Amino-4-cyclopropyl-6-(3-(5-(3-hydroxy-1-methyl-2-oxopyrrolidin-3-yl)isoxazol-3-yl)phenyl)picolinamide

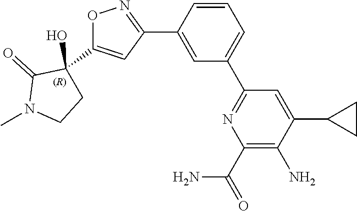

Step A: 3-Amino-6-chloro-4-cyclopropylpicolinamide. A vial was charged with toluene (10 mL) and H₂O (1 mL). The solution was sparged under nitrogen for 15 minutes before the addition of CATACXIUM PD G4 (CAS #2230788-67-5, 237 mg, 0.319 mmol), which formed a light brown mixture. To this mixture, Cs₂CO₃ (2.08 g, 6.38 mmol) was added, which formed a dark brown solution. The brown solution was then stirred while being continuously sparged with nitrogen. Potassium cyclopropyltrifluoroborate (472 mg, 2.19 mmol) was added followed by 3-amino-4,6-dichloropicolinonitrile (401 mg, 2.13 mmol). The vial was then sealed and stirred for 24 hours in the microwave at 90° C. After this time, the solution was filtered over diatomaceous earth and washed with acetone. The resulting filtered solution was evaporated to dryness and purified by FCC (EtOAc/CH₂Cl₂) providing two products: 3-amino-6-chloro-4-cyclopropylpicolinonitrile (180 mg, 43.7%), MS (ESI): Mass calcd. for C₉H₈ClO₃, 193.0; m/z found, 194.1 [M+H]+, and 3-amino-6-chloro-4-cyclopropylpicolinamide (78 mg, 43%), MS (ESI): Mass calcd. for C₉H₁₀ClN₃O, 211.1; m/z found, 212.1 [M+H]⁺.

Step B: (R)-3-Amino-4-cyclopropyl-6-(3-(5-(3-hydroxy-1-methyl-2-oxopyrrolidin-3-yl)isoxazol-3-yl)phenyl)picolinamide. (R)-3-Amino-4-cyclopropyl-6-(3-(5-(3-hydroxy-1-methyl-2-oxopyrrolidin-3-yl)isoxazol-3-yl)phenyl)picolinamide (68 mg, 43%) was prepared in a manner analogous to Example 47 Step B using 3-amino-6-chloro-4-cyclopropylpicolinamide instead of 4-chloro-6-methoxypicolinamide. MS (ESI): Mass calcd. for C₂₃H₂₃N₅O₄, 433.2; m/z found, 434.2 [M+H]⁺. ¹H NMR (500 MHz, CD₃OD) δ 8.42 (t, J=1.8 Hz, 1H), 8.12-8.00 (m, 1H), 7.80-7.72 (m, 1H), 7.59 (d, J=0.8 Hz, 1H), 7.53 (t, J=7.8 Hz, 1H), 6.99 (s, 1H), 3.65-3.49 (m, 2H), 2.97 (s, 3H), 2.81-2.66 (m, 1H), 2.47-2.34 (m, 1H), 1.86-1.70 (m, 1H), 1.15-1.02 (m, 2H), 0.82-0.70 (m, 2H).

### Example 89: (R)-3-Amino-5-fluoro-6-(3-(5-(3-hydroxy-1-methyl-2-oxopyrrolidin-3-yl)isoxazol-3-yl)phenyl)picolinamide

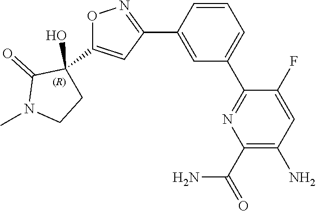

Step A: 3-Amino-6-bromo-5-fluoropicolinamide. To a colorless solution of 3-amino-6-bromo-5-fluoropicolinic acid (247 mg, 105 mmol) in THF (10 mL) at 0° C. was added ClCO₂Et (160 mg, 1.47 mmol) and Et₃N (0.219 mL, 1.58 mmol) The mixture was stirred at this temperature for 30 minutes. An aqueous solution of NH₄Cl (1.5 mL, 1.0 M) was added, then H₂O (5 mL) was added. The resulting mixture was extracted with EtOAc (30 mL×2). The combined organic layers were dried over anhydrous MgSO₄, filtered and concentrated to provide the title compound (210 mg, 85%). MS (ESI): Mass calcd. for C₆H₅BrFN₃O, 233.0; m/z found, 234.0 [M+H]⁺.

Step B: (R)-3-Amino-5-fluoro-6-(3-(5-(3-hydroxy-1-methyl-2-oxopyrrolidin-3-yl)isoxazol-3-yl)phenyl)picolinamide. (R)-3-Amino-5-fluoro-6-(3-(5-(3-hydroxy-1-methyl-2-oxopyrrolidin-3-yl)isoxazol-3-yl)phenyl)picolinamide (50 mg, 23%) was prepared in a manner analogous to Example 47 Step B using 3-amino-6-bromo-5-fluoropicolinamide instead of 4-chloro-6-methoxypicolinamide. MS (ESI): Mass calcd. for C₂₀H₁₈FN₅O₄, 411.1; m/z found, 412.1 [M+H]⁺. ¹H NMR (500 MHz, CD₃OD) δ 8.32 (q, J=1.6 Hz, 1H), 8.04-7.92 (m, 1H), 7.83-7.73 (m, 1H), 7.53 (t, J=7.8 Hz, 1H), 7.01-6.88 (m, 2H), 3.63-3.48 (m, 2H), 2.96 (s, 3H), 2.80-2.67 (m, 1H), 2.47-2.33 (m, 1H).

### Example 90: (R)-1-Ethyl-6-(3-(5-(3-hydroxy-1-methyl-2-oxopyrrolidin-3-yl)isoxazol-3-yl)phenyl)-1H-imidazo[4,5-c]pyridine-4-carboxamide

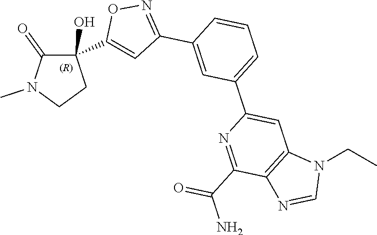

Step A: 6-Chloro-1-ethyl-1H-imidazo[4,5-c]pyridine-4-carbonitrile. A mixture of 6-chloro-1H-imidazo[4,5-c]pyridine-4-carbonitrile (178 mg, 0.997 mmol), ethyl iodide (164 mg, 1.05 mmol), and K₂CO₃ (275 mg, 1.99 mmol) in DMF (1 mL) was stirred for 8 h at RT. Water (20 mL) was added. The solid was filtered and dried to provide the title compound (120 mg, 58%). MS (ESI): Mass calcd. for C₉H₇ClN₄, 206.0; m/z found, 207.1 [M+H]⁺.

Step B: (R)-1-Ethyl-6-(3-(5-(3-hydroxy-1-methyl-2-oxopyrrolidin-3-yl)isoxazol-3-yl)phenyl)-1H-imidazo[4,5-c]pyridine-4-carbonitrile. (R)-1-Ethyl-6-(3-(5-(3-hydroxy-1-methyl-2-oxopyrrolidin-3-yl)isoxazol-3-yl)phenyl)-1H-imidazo[4,5-c]pyridine-4-carbonitrile (50 mg, 24%) was prepared in a manner analogous to Example 47 Step B using 6-chloro-1-ethyl-1H-imidazo[4,5-c]pyridine-4-carbonitrile instead of 4-chloro-6-methoxypicolinamide. MS (ESI): Mass calcd. for C₂₃H₂₀N₆O₃, 428.1; m/z found, 429.1 [M+H]⁺.

Step C: (R)-1-Ethyl-6-(3-(5-(3-hydroxy-1-methyl-2-oxopyrrolidin-3-yl)isoxazol-3-yl)phenyl)-1H-imidazo[4,5-c]pyridine-4-carboxamide. To a mixture of (R)-1-ethyl-6-(3-(5-(3-hydroxy-1-methyl-2-oxopyrrolidin-3-yl)isoxazol-3-yl)phenyl)-1H-imidazo[4,5-c]pyridine-4-carbonitrile (30 mg, 0.07 mmol) in MeOH (2 mL), NaOMe (0.04 mL, 30% in MeOH) was added. The mixture was heated at 130° C. for 16 h. Then it was cooled to RT and concentrated. The residue was purified with HPLC (H₂O/CH₃CN) to provide the title compound (15 mg, 48%). MS (ESI): Mass calcd. for C₂₃H₂₂N₆O₄, 446.2; m/z found, 447.1 [M+H]⁺. ¹H NMR (500 MHz, CD₃OD) δ 9.44 (s, 1H), 8.68 (d, J=13.0 Hz, 2H), 8.46-8.32 (m, 1H), 7.93 (d, J=7.6 Hz, 1H), 7.66 (t, J=7.8 Hz, 1H), 7.08 (s, 1H), 4.63 (q, J=7.3 Hz, 2H), 3.70-3.52 (m, 2H), 2.98 (d, J=0.9 Hz, 3H), 2.85-2.69 (m, 1H), 2.54-2.37 (m, 1H), 1.68 (t, J=7.3 Hz, 3H).

### Example 91: (R)-6-(3-(5-(3-Hydroxy-1-methyl-2-oxopyrrolidin-3-yl)isoxazol-3-yl)phenyl)-4-(trifluoromethyl)picolinamide

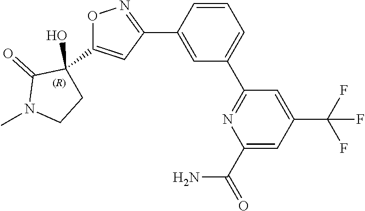

(R)-6-(3-(5-(3-Hydroxy-1-methyl-2-oxopyrrolidin-3-yl)isoxazol-3-yl)phenyl)-4-(trifluoromethyl)picolinamide (90 mg) was prepared in a manner analogous to Example 89 using 6-chloro-4-(trifluoromethyl)picolinic acid instead of 3-amino-6-bromo-5-fluoropicolinic acid in Step A. MS (ESI): Mass calcd. for C₂₁H₁₇F₃N₄O₄, 446.1; m/z found, 447.1 [M+H]⁺. ¹H NMR (500 MHz, CD₃OD) δ 8.68 (t, J=1.8 Hz, 1H), 8.40 (dd, J=1.6, 0.8 Hz, 1H), 8.37-8.24 (m, 2H), 8.00-7.91 (m, 1H), 7.65 (t, J=7.8 Hz, 1H), 7.07 (s, 1H), 3.67-3.51 (m, 2H), 2.98 (s, 3H), 2.83-2.70 (m, 1H), 2.49-2.33 (m, 1H).

### Example 92: (R)-2-(3-(5-(3-Hydroxy-1-methyl-2-oxopyrrolidin-3-yl)isoxazol-3-yl)phenyl) imidazo[1,2-a]pyridine-8-carboxamide

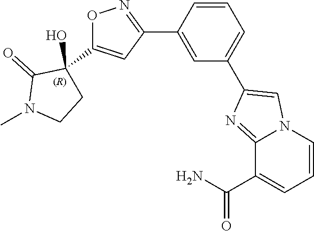

(R)-2-(3-(5-(3-Hydroxy-1-methyl-2-oxopyrrolidin-3-yl)isoxazol-3-yl)phenyl)imidazo[1,2-a]pyridine-8-carboxamide (130 mg) was prepared in a manner analogous to Example 47 using methyl 2-bromoimidazo[1,2-a]pyridine-8-carboxylate instead of methyl 4-chloro-6-methoxypicolinate in Step A. MS (ESI): Mass calcd. for C₂₂H₁₉N₅O₄, 417.1; m/z found, 418.1 [M+H]⁺. ¹H NMR (500 MHz, CD₃OD) δ 8.59 (dd, J=6.7, 1.3 Hz, 1H), 8.44 (t, J=1.6 Hz, 1H), 8.37 (s, 1H), 8.15-8.06 (m, 2H), 7.82-7.73 (m, 1H), 7.61-7.50 (m, 1H), 7.03 (t, J=7.0 Hz, 1H), 6.96 (s, 1H), 3.65-3.49 (m, 2H), 2.98 (s, 3H), 2.81-2.70 (m, 1H), 2.51-2.33 (m, 1H).

### Example 93: (R)-1-(3-(5-(3-Hydroxy-1-methyl-2-oxopyrrolidin-3-yl)isoxazol-3-yl)phenyl)-5,6,7,8-tetrahydroimidazo[1,5-a]pyridine-3-carboxamide

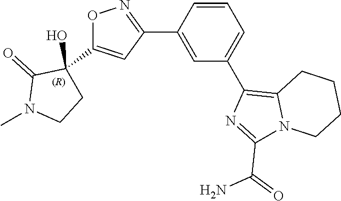

(R)-1-(3-(5-(3-Hydroxy-1-methyl-2-oxopyrrolidin-3-yl)isoxazol-3-yl)phenyl)-5,6,7,8-tetrahydroimidazo[1,5-a]pyridine-3-carboxamide (70 mg) was prepared in a manner analogous to Example 89 using 1-bromo-5,6,7,8-tetrahydroimidazo[1,5-a]pyridine-3-carboxylic acid instead of 3-amino-6-bromo-5-fluoropicolinic acid in Step A. MS (ESI): Mass calcd. for C₂₂H₂₃N₅O₄, 421.2; m/z found, 422.2 [M+H]⁺. ¹H NMR (500 MHz, CD₃OD) δ 8.11 (d, J=1.8 Hz, 1H), 7.90-7.79 (m, 1H), 7.79-7.70 (m, 1H), 7.58 (t, J=7.8 Hz, 1H), 6.93 (s, 1H), 4.50 (t, J=6.2 Hz, 2H), 3.65-3.49 (m, 2H), 3.04 (t, J=6.4 Hz, 2H), 2.96 (s, 3H), 2.80-2.66 (m, 1H), 2.45-2.35 (m, 1H), 2.10-1.98 (m, 2H), 1.98-1.85 (m, 2H).

### Example 94: (R)-4-(3-(5-(3-Hydroxy-1-methyl-2-oxopyrrolidin-3-yl)isoxazol-3-yl)phenyl)-1H-pyrrolo[2,3-b]pyridine-6-carboxamide

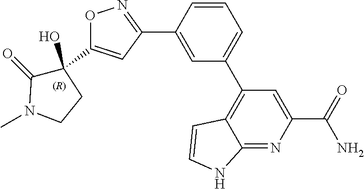

(R)-4-(3-(5-(3-Hydroxy-1-methyl-2-oxopyrrolidin-3-yl)isoxazol-3-yl)phenyl)-1H-pyrrolo[2,3-b]pyridine-6-carboxamide (42 mg) was prepared in a manner analogous to Example 47 using methyl 4-chloro-1H-pyrrolo[2,3-b]pyridine-6-carboxylate instead of methyl 4-chloro-6-methoxypicolinate in Step A. MS (ESI): Mass calcd. for C₂₂H₁₉N₅O₄, 417.1; m/z found, 418.2 [M+H]⁺. ¹H NMR (500 MHz, CD₃OD) δ 8.43-8.19 (m, 1H), 8.18-7.83 (m, 3H), 7.80-7.50 (m, 2H), 7.07-6.92 (m, 1H), 6.87-6.58 (m, 1H), 3.64-3.50 (m, 2H), 2.96 (s, 3H), 2.81-2.70 (m, 1H), 2.47-2.31 (m, 1H).

### Example 95: (R)-1-(3-(5-(3-Hydroxy-1-methyl-2-oxopyrrolidin-3-yl)isoxazol-3-yl)phenyl)-4,5,6,7-tetrahydro-1H-indazole-3-carboxylic acid

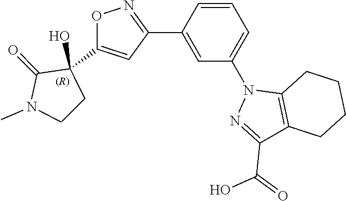

Step A: Methyl (R)-1-(3-(5-(3-hydroxy-1-methyl-2-oxopyrrolidin-3-yl)isoxazol-3-yl)phenyl)-4,5,6,7-tetrahydro-1H-indazole-3-carboxylate. A mixture of methyl 4,5,6,7-tetrahydro-1H-indazole-3-carboxylate (225 mg, 1.25 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 670 mg, 1.74 mmol), Cu(OAc)₂ (68 mg, 0.38 mmol), and pyridine (0.312 mL, 3.88 mmol) in DMF (2.5 mL) was heated at 92° C. for 3 h, and cooled to RT. H₂O (50 mL) was added and the mixture was extracted with EtOAc (3×30 mL). The organic extracts were combined and concentrated. The residue was purified with FCC (5% MeOH/CH₂Cl₂) and provided the title compound (50 mg, 9%). MS (ESI): Mass calcd. for C₂₃H₂₄N₄O₅, 436.2; m/z found, 437.2 [M+H]⁺.

Step B: (R)-1-(3-(5-(3-Hydroxy-1-methyl-2-oxopyrrolidin-3-yl)isoxazol-3-yl)phenyl)-4,5,6,7-tetrahydro-1H-indazole-3-carboxylic acid. A mixture of methyl (R)-1-(3-(5-(3-hydroxy-1-methyl-2-oxopyrrolidin-3-yl)isoxazol-3-yl)phenyl)-4,5,6,7-tetrahydro-1H-indazole-3-carboxylate (50 mg, 0.12 mmol) in NH₃ (10 mL, 2 M in MeOH) was heated at 80° C. for 16 h. Aqueous NH₄OH (5 N, 5 mL) was added and the mixture was heated at 150° C. for 3 h. Then, the reaction mixture was cooled to RT and concentrated. The residue was purified with reverse HPLC (CH₃CN/H₂O) to provide the title compound (21 mg, 43%). MS (ESI): Mass calcd. for C₂₂H₂₂N₄O₅, 422.2; m/z found, 423.2 [M+H]⁺. ¹H NMR (500 MHz, CD₃OD) δ 7.98-7.82 (m, 2H), 7.57 (t, J=7.8 Hz, 1H), 7.52-7.42 (m, 1H), 6.91 (s, 1H), 3.60-3.50 (m, 2H), 2.95 (s, 3H), 2.84 (t, J=6.1 Hz, 2H), 2.76-2.65 (m, 3H), 2.46-2.27 (m, 1H), 1.92-1.77 (m, 4H).

### Example 96: (R)-8-Amino-6-(3-(5-(3-hydroxy-1-methyl-2-oxopyrrolidin-3-yl)isoxazol-3-yl)phenyl)pyrido[3,2-d]pyrimidin-4(3H)-one

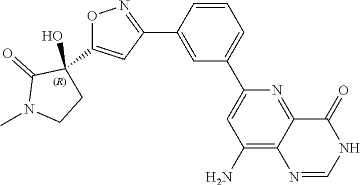

Step A: (R)-6-(3-(5-(3-Hydroxy-1-methyl-2-oxopyrrolidin-3-yl)isoxazol-3-yl)phenyl)-1-((2-(trimethylsilyl)ethoxy)methyl)-1H-imidazo[4,5-c]pyridine-4-carbonitrile. (R)-6-(3-(5-(3-Hydroxy-1-methyl-2-oxopyrrolidin-3-yl)isoxazol-3-yl)phenyl)-1-((2-(trimethylsilyl)ethoxy)methyl)-1H-imidazo[4,5-c]pyridine-4-carbonitrile (130 mg) was prepared in a manner analogous to Example 47 (Step A and Step B) using 2-(trimethylsilyl)ethoxymethyl chloride instead of ethyl iodide. MS (ESI): Mass calcd. for C₂₇H₃₀N₆O₄Si, 530.6; m/z found, 531.2 [M+H]⁺. ¹H NMR (500 MHz, CD₃OD) δ 8.73-8.64 (m, 1H), 8.62-8.49 (m, 2H), 8.32-8.09 (m, 1H), 7.99-7.90 (m, 1H), 7.70-7.60 (m, 1H), 7.12-7.00 (m, 1H), 5.95-5.70 (m, 2H), 3.77-3.54 (m, 4H), 3.09-2.95 (m, 3H), 2.91-2.76 (m, 1H), 2.57-2.37 (m, 1H), 1.04-0.89 (m, 2H), 0.04-−0.14 (m, 9H).

Step B: (R)-8-Amino-6-(3-(5-(3-hydroxy-1-methyl-2-oxopyrrolidin-3-yl)isoxazol-3-yl)phenyl)pyrido[3,2-d]pyrimidin-4(3H)-one. To a solution of (R)-6-(3-(5-(3-hydroxy-1-methyl-2-oxopyrrolidin-3-yl)isoxazol-3-yl)phenyl)-1-((2-(trimethylsilyl)ethoxy)methyl)-1H-imidazo[4,5-c]pyridine-4-carbonitrile (60 mg, 0.1 mmol) in MeOH (2 mL), NaOMe (0.062 mL, 0.34 mmol, 30% in MeOH) and TBAF (0.11 mL, 0.11 mmol, 1 M in THF) were added. The mixture was heated at 130° C. for 16 h, then cooled to RT and concentrated. The residue was partitioned between with EtOAc/H₂O (15/7 mL). The organic layer was separated and concentrated. The HPLC purification (CH₃CN/H₂O) provided two products: the title compound (18 mg, 38%) and (R)-6-(3-(5-(3-hydroxy-1-methyl-2-oxopyrrolidin-3-yl)isoxazol-3-yl)phenyl)-1H-imidazo[4,5-c]pyridine-4-carboxamide (Example 97) (7 mg, 18%). MS (ESI): Mass calcd. for C₂₁H₁₈N₆O₄, 418.1; m/z found, 419.1 [M+H]⁺. ¹H NMR (600 MHz, CD₃OD) δ 8.55 (s, 1H), 8.18-8.06 (m, 1H), 8.00 (s, 1H), 7.90 (d, J=7.7 Hz, 1H), 7.56 (t, J=7.8 Hz, 1H), 7.40 (s, 1H), 7.07 (s, 1H), 3.64-3.52 (m, 2H), 2.97 (s, 3H), 2.78-2.68 (m, 1H), 2.47-2.34 (m, 1H).

### Example 97: (R)-6-(3-(5-(3-Hydroxy-1-methyl-2-oxopyrrolidin-3-yl)isoxazol-3-yl)phenyl)-1H-imidazo[4,5-c]pyridine-4-carboxamide

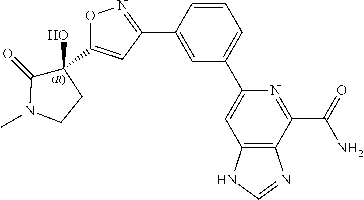

(R)-6-(3-(5-(3-Hydroxy-1-methyl-2-oxopyrrolidin-3-yl)isoxazol-3-yl)phenyl)-1H-imidazo[4,5-c]pyridine-4-carboxamide (7 mg) was prepared as described in Example 96. MS (ESI): Mass calcd. for C₂₁H₁₈N₆O₄, 418.1; m/z found, 419.1 [M+H]⁺. ¹H NMR (500 MHz, CD₃OD) δ 8.73-8.59 (m, 1H), 8.53-8.21 (m, 3H), 7.95-7.81 (m, 1H), 7.65-7.50 (m, 1H), 7.09-6.98 (m, 1H), 4.56 (s, 1H), 3.68-3.51 (m, 2H), 2.98 (s, 3H), 2.82-2.68 (m, 1H), 2.50-2.31 (m, 1H).

### Example 98: (R)-2-(3-(5-(3-Hydroxy-1-methyl-2-oxopyrrolidin-3-yl)isoxazol-3-yl)phenyl)-6-(trifluoromethyl)pyrimidine-4-carboxamide

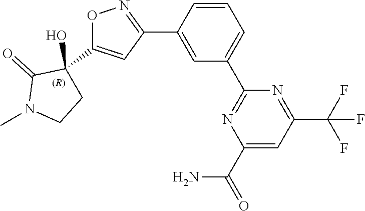

Step A: (R)-2-(3-(5-(3-Hydroxy-1-methyl-2-oxopyrrolidin-3-yl)isoxazol-3-yl)phenyl)-6-(trifluoromethyl)pyrimidine-4-carboxylic acid. (R)-2-(3-(5-(3-Hydroxy-1-methyl-2-oxopyrrolidin-3-yl)isoxazol-3-yl)phenyl)-6-(trifluoromethyl)pyrimidine-4-carboxylic acid (136 mg, 55%) was prepared in a manner analogous to Example 47 Step B using ethyl 2-chloro-6-(trifluoromethyl)pyrimidine-4-carboxylate instead of 4-chloro-6-methoxypicolinamide. MS (ESI): Mass calcd. for C₂₀H₁₅F₃N₄O₅, 448.1; m/z found, 449.1 [M+H]⁺. ¹H NMR (500 MHz, CD₃OD) δ 9.14 (s, 1H), 8.73 (d, J=7.8 Hz, 1H), 8.17 (s, 1H), 8.08 (d, J=7.7 Hz, 1H), 7.66 (t, J=7.4 Hz, 1H), 7.12 (s, 1H), 3.58 (dd, J=7.4, 5.8 Hz, 2H), 2.97 (s, 3H), 2.80-2.70 (m, 1H), 2.49-2.37 (m, 1H).

Step B: (R)-2-(3-(5-(3-Hydroxy-1-methyl-2-oxopyrrolidin-3-yl)isoxazol-3-yl)phenyl)-6-(trifluoromethyl)pyrimidine-4-carboxamide. (R)-2-(3-(5-(3-Hydroxy-1-methyl-2-oxopyrrolidin-3-yl)isoxazol-3-yl)phenyl)-6-(trifluoromethyl)pyrimidine-4-carboxamide (40 mg, 67%) was prepared in a manner analogous to Example 89 Step A using (R)-2-(3-(5-(3-hydroxy-1-methyl-2-oxopyrrolidin-3-yl)isoxazol-3-yl)phenyl)-6-(trifluoromethyl)pyrimidine-4-carboxylic acid instead of 3-amino-6-bromo-5-fluoropicolinic acid. MS (ESI): Mass calcd. for C₂₀H₁₆F₃N₅O₄, 447.1; m/z found, 448.1 [M+H]⁺. ¹H NMR (500 MHz, CD₃OD) δ 9.00 (t, J=1.9 Hz, 1H), 8.71-8.58 (m, 1H), 8.27 (s, 1H), 8.07-7.95 (m, 1H), 7.65 (t, J=7.8 Hz, 1H), 7.04 (s, 1H), 3.70-3.50 (m, 2H), 2.98 (s, 3H), 2.83-2.71 (m, 1H), 2.51-2.35 (m, 1H).

### Example 99: (R)-1-(3-(5-(3-Hydroxy-1-methyl-2-oxopyrrolidin-3-yl)isoxazol-3-yl)phenyl)-5-(1-methyl-1H-pyrazol-3-yl)-1H-indazole-3-carboxamide

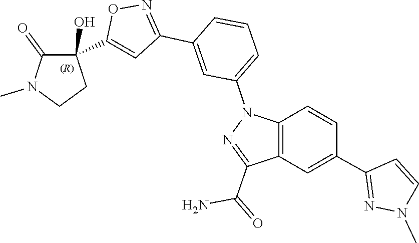

Step A: Ethyl (R)-5-bromo-1-(3-(5-(3-hydroxy-1-methyl-2-oxopyrrolidin-3-yl)isoxazol-3-yl)phenyl)-1H-indazole-3-carboxylate. Ethyl (R)-5-bromo-1-(3-(5-(3-hydroxy-1-methyl-2-oxopyrrolidin-3-yl)isoxazol-3-yl)phenyl)-1H-indazole-3-carboxylate (100 mg, 19%) was prepared in a manner analogous to Example 95 Step A using ethyl 5-bromo-1H-indazole-3-carboxylate instead of methyl 4,5,6,7-tetrahydro-1H-indazole-3-carboxylate. MS (ESI): Mass calcd. for C₂₄H₂₁BrN₄O₅, 524.1; m/z found, 525.0 [M+H]⁺.

Step B: Ethyl (R)-1-(3-(5-(3-hydroxy-1-methyl-2-oxopyrrolidin-3-yl)isoxazol-3-yl)phenyl)-5-(1-methyl-1H-pyrazol-3-yl)-1H-indazole-3-carboxylate. Ethyl (R)-1-(3-(5-(3-hydroxy-1-methyl-2-oxopyrrolidin-3-yl)isoxazol-3-yl)phenyl)-5-(1-methyl-1H-pyrazol-3-yl)-1H-indazole-3-carboxylate (50 mg, 50%) was prepared in a manner analogous to Example 47 Step B using ethyl (R)-5-bromo-1-(3-(5-(3-hydroxy-1-methyl-2-oxopyrrolidin-3-yl)isoxazol-3-yl)phenyl)-1H-indazole-3-carboxylate and 1-methyl-3-(4,4,5,5-tetramethyl-1,3,2-dioxaborolan-2-yl)-1H-pyrazole instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₈H₂₆N₆O₅, 526.1; m/z found, 527.1 [M+H]⁺.

Step C: (R)-1-(3-(5-(3-Hydroxy-1-methyl-2-oxopyrrolidin-3-yl)isoxazol-3-yl)phenyl)-5-(1-methyl-1H-pyrazol-3-yl)-1H-indazole-3-carboxamide. (R)-1-(3-(5-(3-Hydroxy-1-methyl-2-oxopyrrolidin-3-yl)isoxazol-3-yl)phenyl)-5-(1-methyl-1H-pyrazol-3-yl)-1H-indazole-3-carboxamide (18 mg, 36%) was prepared in a manner analogous to Example 47 Step A using ethyl (R)-1-(3-(5-(3-hydroxy-1-methyl-2-oxopyrrolidin-3-yl)isoxazol-3-yl)phenyl)-5-(1-methyl-1H-pyrazol-3-yl)-1H-indazole-3-carboxylate instead of methyl 4-chloro-6-methoxypicolinate. MS (ESI): Mass calcd. for C₂₆H₂₃N₇O₄, 497.2; m/z found, 498.3 [M+H]⁺. ¹H NMR (500 MHz, CD₃OD) δ 8.72 (dd, J=1.6, 0.8 Hz, 1H), 8.32 (t, J=1.9 Hz, 1H), 8.04 (dd, J=8.9, 1.7 Hz, 1H), 8.02-7.93 (m, 2H), 7.91 (dd, J=8.9, 0.8 Hz, 1H), 7.76 (t, J=7.9 Hz, 1H), 7.64 (d, J=2.3 Hz, 1H), 7.02 (s, 1H), 6.72 (d, J=2.3 Hz, 1H), 3.96 (s, 3H), 3.62-3.55 (m, 2H), 2.97 (s, 3H), 2.81-2.72 (m, 1H), 2.48-2.37 (m, 1H).

### Example 100: (R)-4-(3,3-Difluoroazetidin-1-yl)-6-(3-(5-(3-hydroxy-1-methyl-2-oxopyrrolidin-3-yl)isoxazol-3-yl)phenyl)picolinamide

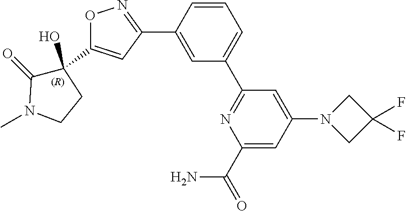

Step A: Methyl 6-chloro-4-(3,3-difluoroazetidin-1-yl)picolinate. To 3,3-difluoroazetidine hydrochloride (259 mg, 2.00 mmol) and methyl 4,6-dichloropicolinate (412 mg, 2 mmol) in DMA (1.5 mL), Et₃N (1.39 mL, 10 mmol) was added. The mixture was heated at 80° C. for 16 h. Solids were formed, filtered, collected and dried to provide the title compound (410 mg, 78%). MS (ESI): Mass calcd. for C₁₀H₉ClF₂N₂O₂, 262.1; m/z found, 263.1 [M+H]⁺.

Step B: (R)-4-(3,3-Difluoroazetidin-1-yl)-6-(3-(5-(3-hydroxy-1-methyl-2-oxopyrrolidin-3-yl)isoxazol-3-yl)phenyl)picolinamide. (R)-4-(3,3-Difluoroazetidin-1-yl)-6-(3-(5-(3-hydroxy-1-methyl-2-oxopyrrolidin-3-yl)isoxazol-3-yl)phenyl)picolinamide (65 mg) was prepared in a manner analogous to Example 47 using methyl 6-chloro-4-(3,3-difluoroazetidin-1-yl)picolinate instead of methyl 4-chloro-6-methoxypicolinate. MS (ESI): Mass calcd. for C₂₃H₂₁F₂N₅O₄, 469.2; m/z found, 470.1 [M+H]⁺. ¹H NMR (600 MHz, CD₃OD) δ 8.33 (d, J=1.9 Hz, 1H), 8.13-8.02 (m, 1H), 7.98 (d, J=7.7 Hz, 1H), 7.70 (t, J=7.8 Hz, 1H), 7.34 (d, J=2.3 Hz, 1H), 7.12 (d, J=2.4 Hz, 1H), 7.01 (s, 1H), 4.74 (t, J=11.6 Hz, 4H), 3.66-3.50 (m, 2H), 2.79-2.69 (m, 1H), 2.47-2.35 (m, 1H).

### Example 101: (R)-5-Fluoro-1-(3-(5-(3-hydroxy-1-methyl-2-oxopyrrolidin-3-yl)isoxazol-3-yl)phenyl)-1H-indazole-3-carboxamide

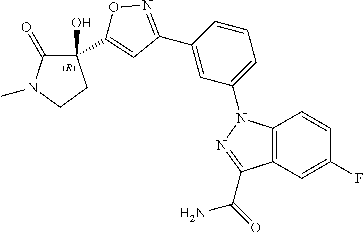

(R)-5-Fluoro-1-(3-(5-(3-hydroxy-1-methyl-2-oxopyrrolidin-3-yl)isoxazol-3-yl)phenyl)-1H-indazole-3-carboxamide (50 mg) was prepared in a manner analogous to Example 95 using ethyl 5-fluoro-1H-indazole-3-carboxylate instead of methyl 4,5,6,7-tetrahydro-1H-indazole-3-carboxylate. MS (ESI): Mass calcd. for C₂₂H₁₈FN₅O₄, 435.1; m/z found, 436.3 [M+H]⁺. ¹H NMR (500 MHz, CD₃OD) δ 8.24 (t, J=1.9 Hz, 1H), 7.98-7.82 (m, 4H), 7.71 (t, J=7.9 Hz, 1H), 7.39-7.29 (m, 1H), 7.00 (s, 1H), 3.62-3.52 (m, 2H), 2.96 (s, 3H), 2.82-2.69 (m, 1H), 2.47-2.35 (m, 1H).

### Example 102: (R)-1-(3-(5-(3-Hydroxy-1-methyl-2-oxopyrrolidin-3-yl)isoxazol-3-yl)phenyl)-4,5,6,7-tetrahydro-1H-indazole-3-carboxamide

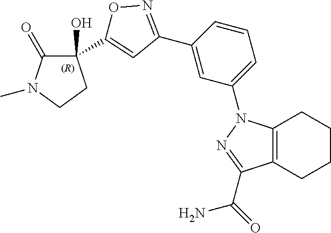

(R)-1-(3-(5-(3-Hydroxy-1-methyl-2-oxopyrrolidin-3-yl)isoxazol-3-yl)phenyl)-4,5,6,7-tetrahydro-1H-indazole-3-carboxamide (62 mg) was prepared in a manner analogous to Example 89 Step A using (R)-1-(3-(5-(3-hydroxy-1-methyl-2-oxopyrrolidin-3-yl)isoxazol-3-yl)phenyl)-4,5,6,7-tetrahydro-1H-indazole-3-carboxylic acid (Example 95) instead of 3-amino-6-bromo-5-fluoropicolinic acid. MS (ESI): Mass calcd. for C₂₂H₂₃N₅O₄, 421.2; m/z found, 422.3 [M+H]⁺. ¹H NMR (500 MHz, CD₃OD) δ 8.07 (t, J=1.8 Hz, 1H), 7.94-7.87 (m, 1H), 7.75-7.59 (m, 2H), 6.97 (s, 1H), 3.63-3.51 (m, 2H), 2.96 (s, 3H), 2.87-2.67 (m, 5H), 2.45-2.38 (m, 1H), 1.88-1.78 (m, 4H).

### Example 103: (R)-1-(3-(5-(3-Hydroxy-1-methyl-2-oxopyrrolidin-3-yl)isoxazol-3-yl)phenyl)-5-methoxy-1H-indazole-3-carboxamide

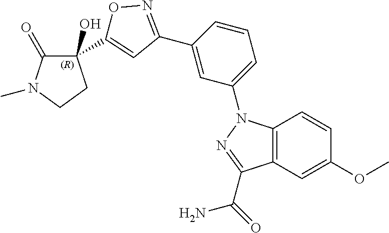

(R)-1-(3-(5-(3-Hydroxy-1-methyl-2-oxopyrrolidin-3-yl)isoxazol-3-yl)phenyl)-5-methoxy-1H-indazole-3-carboxamide (110 mg) was prepared in a manner analogous to Example 89 Step A using (R)-1-(3-(5-(3-hydroxy-1-methyl-2-oxopyrrolidin-3-yl)isoxazol-3-yl)phenyl)-5-methoxy-1H-indazole-3-carboxylic acid (prepared in a manner analogous to Example 95 using ethyl 5-methoxy-1H-indazole-3-carboxylate instead of methyl 4,5,6,7-tetrahydro-1H-indazole-3-carboxylate) instead of 3-amino-6-bromo-5-fluoropicolinic acid. MS (ESI): Mass calcd. for C₂₃H₂₁N₅O₅, 447.1; m/z found, 448.3 [M+H]⁺. ¹H NMR (500 MHz, CD₃OD) δ 8.19 (t, J=1.9 Hz, 1H), 7.90-7.82 (m, 2H), 7.73-7.61 (m, 3H), 7.12 (dd, J=9.2, 2.5 Hz, 1H), 6.98 (s, 1H), 3.87 (s, 3H), 3.63-3.54 (m, 2H), 2.96 (s, 3H), 2.79-2.67 (m, 1H), 2.47-2.32 (m, 1H).

### Example 104: (R)-6-(3-(5-(3-Hydroxy-1-methyl-2-oxopyrrolidin-3-yl)isoxazol-3-yl)phenyl)-3-(methylamino)picolinamide

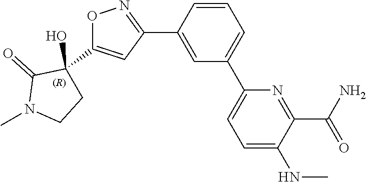

Step A: 6-chloro-3-(methylamino)picolinamide. Methylamine (3 mL, 2 N in THF) and 6-chloro-3-fluoropyridine-2-carboxylic acid (175 mg, 1 mmol) in THF (1.5 mL) were mixed at RT. The mixture was heated at 110° C. for 3 h. After it cooled to RT, aqueous HCl solution (1 N) was added to adjust the pH to pH 3. Solids were formed, filtered off and dried to provide the title compound (90 mg, 48%). MS (ESI): Mass calcd. for C₇H₈ClN₃O, 185.0; m/z found, 186.3 [M+H]⁺.

Step B: (R)-6-(3-(5-(3-Hydroxy-1-methyl-2-oxopyrrolidin-3-yl)isoxazol-3-yl)phenyl)-3-(methylamino)picolinamide. (R)-6-(3-(5-(3-Hydroxy-1-methyl-2-oxopyrrolidin-3-yl)isoxazol-3-yl)phenyl)-3-(methylamino)picolinamide (7 mg) was prepared in a manner analogous to Example 89 (step B) using 6-chloro-3-(methylamino)picolinamide instead of (R)-3-Amino-5-fluoro-6-(3-(5-(3-hydroxy-1-methyl-2-oxopyrrolidin-3-yl)isoxazol-3-yl)phenyl)picolinamide. MS (ESI): Mass calcd. for C₂₁H₂₁N₅O₄, 407.2; m/z found, 408.3 [M+H]⁺. ¹H NMR (500 MHz, CD₃OD) δ 8.53-8.42 (m, 1H), 8.16-8.06 (m, 1H), 7.97 (d, J=8.9 Hz, 1H), 7.81-7.74 (m, 1H), 7.61-7.50 (m, 1H), 7.29 (d, J=8.9 Hz, 1H), 6.99 (s, 1H), 3.67-3.52 (m, 2H), 3.02-2.90 (m, 6H), 2.82-2.71 (m, 1H), 2.48-2.36 (m, 1H).

### Example 105: (R)-2-(3-(5-(3-Hydroxy-1-methyl-2-oxopyrrolidin-3-yl)isoxazol-3-yl)phenyl)-5-(oxetan-3-ylamino)pyrimidine-4-carboxamide

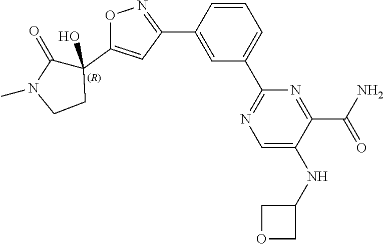

Step A: Ethyl 2-chloro-5-(oxetan-3-ylamino)pyrimidine-4-carboxylate. 3-Oxetanamine (129 mg, 1.77 mmol), ethyl 2-chloro-5-fluoropyrimidine-4-carboxylate (361 mg, 177 mmol) and Et₃N (1.23 mL, 8.83 mmol) in THF (10 mL) were mixed at RT. The mixture was heated at 150° C. for 16 h. The reaction mixture was then cooled to RT and concentrated to provide the title compound (395 mg, 90.0%). MS (ESI): Mass calcd. for C₁₀H₁₂ClN₃O₃, 257.1; m/z found, 258.3 [M+H]⁺.

Step B: (R)-2-(3-(5-(3-Hydroxy-1-methyl-2-oxopyrrolidin-3-yl)isoxazol-3-yl)phenyl)-5-(oxetan-3-ylamino)pyrimidine-4-carboxamide. (R)-2-(3-(5-(3-Hydroxy-1-methyl-2-oxopyrrolidin-3-yl)isoxazol-3-yl)phenyl)-5-(oxetan-3-ylamino)pyrimidine-4-carboxamide (50 mg) was prepared in a manner analogous to Example 47 using ethyl 2-chloro-5-(oxetan-3-ylamino)pyrimidine-4-carboxylate instead of methyl 4-chloro-6-methoxypicolinate. MS (ESI): Mass calcd. for C₂₂H₂₂N₆O₅, 450.2; m/z found, 451.1 [M+H]⁺. ¹H NMR (500 MHz, CD₃OD) δ 8.82 (t, J=1.8 Hz, 1H), 8.51-8.41 (m, 1H), 8.34 (s, 1H), 7.93-7.79 (m, 1H), 7.58 (t, J=7.8 Hz, 1H), 7.00 (s, 1H), 5.07 (t, J=6.7 Hz, 2H), 4.99-4.90 (m, 1H), 4.63 (t, J=6.7 Hz, 2H), 3.66-3.54 (m, 2H), 2.97 (s, 3H), 2.80-2.69 (m, 1H), 2.48-2.35 (m, 1H).

### Example 106: (R)-6-(3-(5-(3-Hydroxy-1-methyl-2-oxopyrrolidin-3-yl)isoxazol-3-yl)phenyl)-3-((oxetan-3-ylmethyl)amino)picolinamide

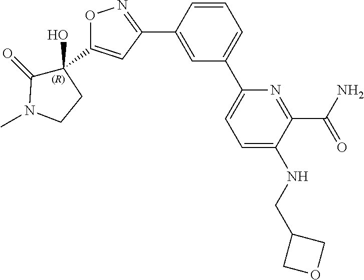

(R)-6-(3-(5-(3-Hydroxy-1-methyl-2-oxopyrrolidin-3-yl)isoxazol-3-yl)phenyl)-3-((oxetan-3-ylmethyl)amino)picolinamide (28 mg) was prepared in a manner analogous to Example 104 using oxetan-3-ylmethanamine instead of methylamine. MS (ESI): Mass calcd. for C₂₄H₂₅N₅O₅, 463.2; m/z found, 464.1 [M+H]⁺. ¹H NMR (500 MHz, CD₃OD) δ 8.53-8.36 (m, 2H), 8.15-8.04 (m, 1H), 7.99-7.86 (m, 1H), 7.81-7.73 (m, 1H), 7.61-7.47 (m, 1H), 7.35 (d, J=8.9 Hz, 1H), 6.99 (d, J=2.1 Hz, 1H), 4.90-4.84 (m, 2H), 4.56-4.41 (m, 2H), 3.64-3.51 (m, 4H), 3.39-3.33 (m, 1H), 2.96 (s, 3H), 2.79-2.64 (m, 1H), 2.46-2.32 (m, 1H).

### Example 107: (R)-2-(3-(5-(3-Hydroxy-1-methyl-2-oxopyrrolidin-3-yl)isoxazol-3-yl)phenyl)-5-((oxetan-3-ylmethyl)amino)pyrimidine-4-carboxamide

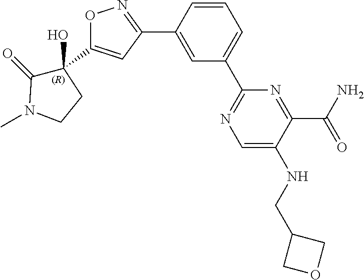

(R)-2-(3-(5-(3-Hydroxy-1-methyl-2-oxopyrrolidin-3-yl)isoxazol-3-yl)phenyl)-5-((oxetan-3-ylmethyl)amino)pyrimidine-4-carboxamide (10 mg) was prepared in a manner analogous to Example 105 using oxetan-3-ylmethanamine instead of 3-oxetanamine. MS (ESI): Mass calcd. for C₂₃H₂₄N₆O₅, 464.2; m/z found, 465.2 [M+H]⁺. ¹H NMR (500 MHz, CD₃OD) δ 8.81 (t, J=1.7 Hz, 1H), 8.64 (s, 1H), 8.53-8.44 (m, 1H), 7.90-7.81 (m, 1H), 7.58 (t, J=7.8 Hz, 1H), 7.00 (s, 1H), 4.92-4.88 (m, 2H), 4.58-4.49 (m, 3H), 3.78-3.66 (m, 2H), 3.63-3.54 (m, 2H), 3.43-3.36 (m, 1H), 2.97 (s, 3H), 2.83-2.70 (m, 1H), 2.46-2.34 (m, 1H).

### Example 108: (R)-3-Amino-4-cyclopropyl-6-(3-(3-(3-hydroxy-1-methyl-2-oxopyrrolidin-3-yl)isoxazol-5-yl)phenyl)picolinamide

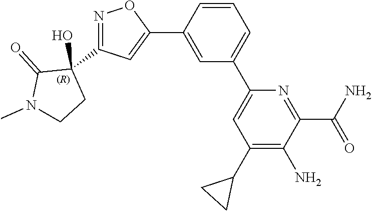

(R)-3-Amino-4-cyclopropyl-6-(3-(3-(3-hydroxy-1-methyl-2-oxopyrrolidin-3-yl)isoxazol-5-yl)phenyl)picolinamide (60 mg) was prepared in a manner analogous to Example 88 using (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₃H₂₃N₅O₄, 433.2; m/z found, 434.1 [M+H]⁺. ¹H NMR (500 MHz, CD₃OD) δ 8.39 (d, J=2.1 Hz, 1H), 8.09 (d, J=8.1 Hz, 1H), 7.78 (d, J=7.4 Hz, 1H), 7.64 (d, J=1.1 Hz, 1H), 7.57 (t, J=7.5 Hz, 1H), 7.05 (s, 1H), 4.55 (s, 1H), 3.60-3.48 (m, 2H), 2.97-2.82 (m, 4H), 2.44-2.14 (m, 1H), 1.86-1.76 (m, 1H), 1.13-1.04 (m, 2H), 0.81-0.71 (m, 2H).

### Example 109: (R)-1-(3-(5-(3-Hydroxy-1-methyl-2-oxopyrrolidin-3-yl)isoxazol-3-yl)phenyl)imidazo[1,5-a]pyrazine-3-carboxamide

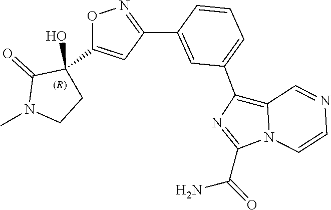

Step A: Ethyl (R)-1-(3-(5-(3-hydroxy-1-methyl-2-oxopyrrolidin-3-yl)isoxazol-3-yl)phenyl)imidazo[1,5-a]pyrazine-3-carboxylate. Ethyl (R)-1-(3-(5-(3-hydroxy-1-methyl-2-oxopyrrolidin-3-yl)isoxazol-3-yl)phenyl)imidazo[1,5-a]pyrazine-3-carboxylate (262 mg, 80%) was prepared in a manner analogous to Example 47 Step B using ethyl 1-bromoimidazo[1,5-a]pyrazine-3-carboxylate instead of 4-chloro-6-methoxypicolinamide. MS (ESI): Mass calcd. for C₂₃H₂₁N₅O₅, 447.2; m/z found, 448.2 [M+H]⁺.

Step B: (R)-1-(3-(5-(3-Hydroxy-1-methyl-2-oxopyrrolidin-3-yl)isoxazol-3-yl)phenyl)imidazo[1,5-a]pyrazine-3-carboxamide. (R)-1-(3-(5-(3-Hydroxy-1-methyl-2-oxopyrrolidin-3-yl)isoxazol-3-yl)phenyl)imidazo[1,5-a]pyrazine-3-carboxamide (87 mg, 36%) was prepared in a manner analogous to Example 47 Step A using ethyl (R)-1-(3-(5-(3-hydroxy-1-methyl-2-oxopyrrolidin-3-yl)isoxazol-3-yl)phenyl)imidazo[1,5-a]pyrazine-3-carboxylate instead of methyl 4-chloro-6-methoxypicolinate. MS (ESI): Mass calcd. for C₂₁H₁₈N₆O₄, 418.1; m/z found, 419.2 [M+H]⁺. ¹H NMR (500 MHz, CD₃OD) δ 9.55-9.41 (m, 1H), 9.26 (dd, J=5.1, 1.6 Hz, 1H), 8.47 (t, J=1.8 Hz, 1H), 8.18-8.06 (m, 1H), 7.96-7.85 (m, 1H), 7.82 (d, J=5.0 Hz, 1H), 7.65 (t, J=7.8 Hz, 1H), 7.00 (s, 1H), 3.67-3.53 (m, 2H), 2.97 (s, 3H), 2.83-2.71 (m, 1H), 2.47-2.34 (m, 1H).

### Example 110: (R)-3-Amino-6-(3-(5-(3-hydroxy-1-methyl-2-oxopyrrolidin-3-yl)isoxazol-3-yl)phenyl)pyrazine-2-carboxamide

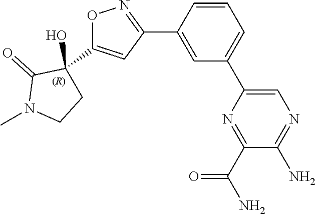

(R)-3-Amino-6-(3-(5-(3-hydroxy-1-methyl-2-oxopyrrolidin-3-yl)isoxazol-3-yl)phenyl)pyrazine-2-carboxamide (16 mg) was prepared in a manner analogous to Example 109 using methyl 3-amino-6-bromopyrazine-2-carboxylate instead of ethyl 1-bromoimidazo[1,5-a]pyrazine-3-carboxylate. MS (ESI): Mass calcd. for C₁₉H₁₈N₆O₄, 394.1; m/z found, 395.2 [M+H]⁺. ¹H NMR (500 MHz, CD₃OD) δ 8.79 (s, 1H), 8.50 (s, 1H), 8.13 (d, J=7.9 Hz, 1H), 7.84 (d, J=7.7 Hz, 1H), 7.59 (t, J=7.8 Hz, 1H), 7.02 (s, 1H), 4.56 (s, 2H), 3.69-3.47 (m, 2H), 2.97 (s, 3H), 2.87-2.70 (m, 1H), 2.41 (s, 1H).

### Example 111: (R)-5-(Cyclopropylamino)-2-(3-(5-(3-hydroxy-1-methyl-2-oxopyrrolidin-3-yl)isoxazol-3-yl)phenyl)pyrimidine-4-carboxamide

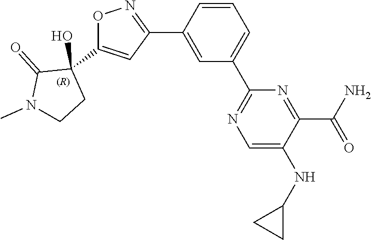

(R)-5-(Cyclopropylamino)-2-(3-(5-(3-hydroxy-1-methyl-2-oxopyrrolidin-3-yl)isoxazol-3-yl)phenyl)pyrimidine-4-carboxamide (26 mg) was prepared in a manner analogous to Example 105 using cyclopropanamine instead of 3-oxetanamine. MS (ESI): Mass calcd. for C₂₂H₂₂N₆O₄, 434.2; m/z found, 435.2 [M+H]⁺. ¹H NMR (500 MHz, CD₃OD) δ 8.92 (d, J=0.6 Hz, 1H), 8.82 (t, J=1.8 Hz, 1H), 8.54-8.44 (m, 1H), 7.91-7.81 (m, 1H), 7.58 (t, J=7.8 Hz, 1H), 7.01 (s, 1H), 3.64-3.50 (m, 2H), 2.97 (s, 3H), 2.80-2.69 (m, 1H), 2.69-2.60 (m, 1H), 2.48-2.35 (m, 1H), 1.02-0.85 (m, 2H), 0.68-0.54 (m, 2H).

### Example 112: (R)-5-Cyclopropyl-1-(3-(5-(3-hydroxy-1-methyl-2-oxopyrrolidin-3-yl)isoxazol-3-yl)phenyl)-1H-indazole-3-carboxamide

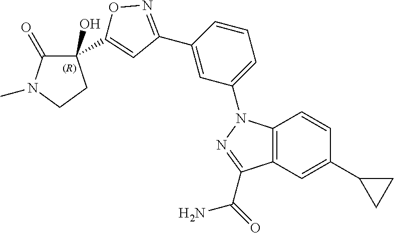

Step A: Ethyl (R)-5-cyclopropyl-1-(3-(5-(3-hydroxy-1-methyl-2-oxopyrrolidin-3-yl)isoxazol-3-yl)phenyl)-1H-indazole-3-carboxylate. Ethyl (R)-5-cyclopropyl-1-(3-(5-(3-hydroxy-1-methyl-2-oxopyrrolidin-3-yl)isoxazol-3-yl)phenyl)-1H-indazole-3-carboxylate (63 mg, 70%) was prepared in a manner analogous to Example 88 Step A using ethyl (R)-5-bromo-1-(3-(5-(3-hydroxy-1-methyl-2-oxopyrrolidin-3-yl)isoxazol-3-yl)phenyl)-1H-indazole-3-carboxylate (Example 99 Step A) instead of 3-amino-4,6-dichloropicolinonitrile. MS (ESI): Mass calcd. for C₂₇H₂₆N₄O₅, 486.2; m/z found, 487.2 [M+H]⁺.

Step B: (R)-5-Cyclopropyl-1-(3-(5-(3-hydroxy-1-methyl-2-oxopyrrolidin-3-yl)isoxazol-3-yl)phenyl)-1H-indazole-3-carboxamide. (R)-5-Cyclopropyl-1-(3-(5-(3-hydroxy-1-methyl-2-oxopyrrolidin-3-yl)isoxazol-3-yl)phenyl)-1H-indazole-3-carboxamide (19 mg, 33%) was prepared in a manner analogous to Example 47 Step A using ethyl (R)-5-cyclopropyl-1-(3-(5-(3-hydroxy-1-methyl-2-oxopyrrolidin-3-yl)isoxazol-3-yl)phenyl)-1H-indazole-3-carboxylate instead of methyl 4-chloro-6-methoxypicolinate. MS (ESI): Mass calcd. for C₂₅H₂₃N₅O₄, 457.2; m/z found, 458.2 [M+H]⁺. ¹H NMR (500 MHz, CD₃OD) δ 8.34-8.18 (m, 1H), 8.11-7.84 (m, 3H), 7.84-7.64 (m, 2H), 7.39-7.23 (m, 1H), 7.09-6.94 (m, 1H), 3.73-3.49 (m, 2H), 2.96 (s, 3H), 2.84-2.63 (m, 1H), 2.53-2.34 (m, 1H), 2.18-1.97 (m, 1H), 1.11-0.95 (m, 2H), 0.90-0.72 (m, 2H).

### Example 113: (R)-3-(3-(5-(4-Amino-8-methoxypyrido[3,2-d]pyrimidin-6-yl)-2,4-difluorophenyl)isoxazol-5-yl)-3-hydroxy-1-methylpyrrolidin-2-one

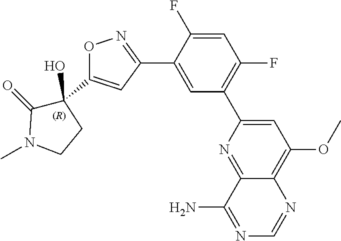

Step A: (R)-3-(3-(2,4-Difluoro-5-(4,4,5,5-tetramethyl-1,3,2-dioxaborolan-2-yl)phenyl)isoxazol-5-yl)-3-hydroxy-1-methylpyrrolidin-2-one. (R)-3-(3-(2,4-Difluoro-5-(4,4,5,5-tetramethyl-1,3,2-dioxaborolan-2-yl)phenyl)isoxazol-5-yl)-3-hydroxy-1-methylpyrrolidin-2-one (1215 mg) was prepared in a manner analogous to Intermediate 3 and Intermediate 4 using 5-bromo-2,4-difluorobenzaldehyde instead of 3-bromobenzaldehyde. MS (ESI): Mass calcd. for C₂₀H₂₃BF₂N₂O₅, 420.2; m/z found, 421.2 [M+H]⁺.

Step B: (R)-3-(3-(5-(4-Amino-8-methoxypyrido[3,2-d]pyrimidin-6-yl)-2,4-difluorophenyl)isoxazol-5-yl)-3-hydroxy-1-methylpyrrolidin-2-one. (R)-3-(3-(5-(4-Amino-8-methoxypyrido[3,2-d]pyrimidin-6-yl)-2,4-difluorophenyl)isoxazol-5-yl)-3-hydroxy-1-methylpyrrolidin-2-one (12 mg, 10%) was prepared in a manner analogous to Example 47 Step A using 6-chloro-8-methoxypyrido[3,2-d]pyrimidin-4-amine (Example 119 Step B) and (R)-3-(3-(2,4-difluoro-5-(4,4,5,5-tetramethyl-1,3,2-dioxaborolan-2-yl)phenyl)isoxazol-5-yl)-3-hydroxy-1-methylpyrrolidin-2-one instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one, respectively. MS (ESI): Mass calcd. for C₂₂H₁₈F₂N₆O₄, 468.1; m/z found, 469.1 [M+H]⁺. ¹H NMR (400 MHz, CD₃OD) δ 8.75 (t, J=8.5 Hz, 1H), 8.39 (s, 1H), 7.77-7.66 (m, 1H), 7.37 (t, J=10.9 Hz, 1H), 6.96 (d, J=3.2 Hz, 1H), 4.14 (s, 4H), 3.64-3.52 (m, 3H), 2.97 (s, 4H), 2.80-2.68 (m, 1H), 2.49-2.35 (m, 1H).

### Example 114: (R)-4-(3-(5-(3-Hydroxy-1-methyl-2-oxopyrrolidin-3-yl)isoxazol-3-yl)phenyl)-1-methyl-1H-imidazole-2-carboxamide

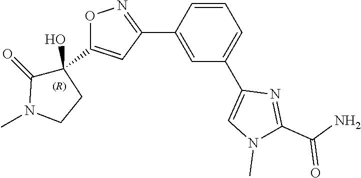

(R)-4-(3-(5-(3-Hydroxy-1-methyl-2-oxopyrrolidin-3-yl)isoxazol-3-yl)phenyl)-1-methyl-1H-imidazole-2-carboxamide (15 mg) was prepared in a manner analogous to Example 47: (R)-4-(3-(5-(3-Hydroxy-1-methyl-2-oxopyrrolidin-3-yl)isoxazol-3-yl)phenyl)-6-methoxypicolinamide. using methyl 4-bromo-1-methyl-1H-imidazole-2-carboxylate instead of methyl 4-chloro-6-methoxypicolinate. MS (ESI): Mass calcd. for C₁₉H₁₉N₅O₄, 381.1; m/z found, 382.3 [M+H]⁺. ¹H NMR (500 MHz, CD₃OD) δ 8.26 (t, J=1.7 Hz, 1H), 7.94-7.84 (m, 1H), 7.78-7.70 (m, 1H), 7.67 (s, 1H), 7.48 (t, J=7.7 Hz, 1H), 6.93 (s, 1H), 4.05 (s, 3H), 3.62-3.51 (m, 2H), 2.96 (s, 3H), 2.79-2.69 (m, 1H), 2.44-2.36 (m, 1H).

### Example 115: (3R,5S)-3-(3-(5-(4-Amino-8-methoxypyrido[3,2-d]pyrimidin-6-yl)-2,4-difluorophenyl)isoxazol-5-yl)-3-hydroxy-1-methyl-5-(trifluoromethyl)pyrrolidin-2-one

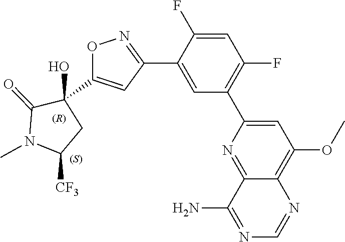

Step A: (3R,5S)-3-(3-(2,4-Difluoro-5-(4,4,5,5-tetramethyl-1,3,2-dioxaborolan-2-yl)phenyl)isoxazol-5-yl)-3-hydroxy-1-methyl-5-(trifluoromethyl)pyrrolidin-2-one (750 mg) was prepared in a manner analogous to Intermediate 3 and 4 using 5-bromo-2,4-difluorobenzaldehyde and (3R,5S)-3-ethynyl-3-hydroxy-1-methyl-5-(trifluoromethyl)pyrrolidin-2-one (Intermediate 45) instead of 3-bromobenzaldehyde and (R)-3-ethynyl-3-hydroxy-1-methylpyrrolidin-2-one. MS (ESI): Mass calcd. for C₂₁H₂₂BF₅N₂O₅, 488.1; m/z found, 489.0 [M+H]⁺.

Step B: (3R,5S)-3-(3-(5-(4-Amino-8-methoxypyrido[3,2-d]pyrimidin-6-yl)-2,4-difluorophenyl)isoxazol-5-yl)-3-hydroxy-1-methyl-5-(trifluoromethyl)pyrrolidin-2-one. (3R,5S)-3-(3-(5-(4-Amino-8-methoxypyrido[3,2-d]pyrimidin-6-yl)-2,4-difluorophenyl)isoxazol-5-yl)-3-hydroxy-1-methyl-5-(trifluoromethyl)pyrrolidin-2-one (57 mg, 14%) was prepared in a manner analogous to Example 47Example 47: (R)-4-(3-(5-(3-Hydroxy-1-methyl-2-oxopyrrolidin-3-yl)isoxazol-3-yl)phenyl)-6-methoxypicolinamide. Step B using 6-chloro-8-methoxypyrido[3,2-d]pyrimidin-4-amine (Example 119 Step B) and (3R,5S)-3-(3-(2,4-difluoro-5-(4,4,5,5-tetramethyl-1,3,2-dioxaborolan-2-yl)phenyl)isoxazol-5-yl)-3-hydroxy-1-methyl-5-(trifluoromethyl)pyrrolidin-2-one instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one, respectively. MS (ESI): Mass calcd. for C₂₃H₁₇F₅N₆O₄, 536.1; m/z found, 537.2 [M+H]⁺. ¹H NMR (500 MHz, CD₃OD) δ 8.80 (t, J=8.4 Hz, 1H), 8.64 (s, 1H), 7.96 (s, 1H), 7.42 (t, J=10.9 Hz, 1H), 7.02 (d, J=3.1 Hz, 1H), 4.54-4.45 (m, 1H), 4.26 (s, 3H), 3.14-3.02 (m, 4H), 2.46 (dd, J=14.7, 4.7 Hz, 1H).

### Example 116: (3R,5S)-3-(3-(3-(4-amino-8-methoxypyrido[3,2-d]pyrimidin-6-yl)-4-fluorophenyl)isoxazol-5-yl)-3-hydroxy-1-methyl-5-(trifluoromethyl)pyrrolidin-2-one

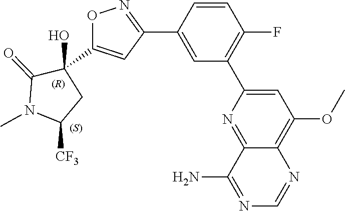

Step A: (3R,5S)-3-(3-(4-Fluoro-3-(4,4,5,5-tetramethyl-1,3,2-dioxaborolan-2-yl)phenyl)isoxazol-5-yl)-3-hydroxy-1-methyl-5-(trifluoromethyl)pyrrolidin-2-one. (3R,5S)-3-(3-(4-Fluoro-3-(4,4,5,5-tetramethyl-1,3,2-dioxaborolan-2-yl)phenyl)isoxazol-5-yl)-3-hydroxy-1-methyl-5-(trifluoromethyl)pyrrolidin-2-one (750 mg) was prepared in a manner analogous to Intermediate 3 and 4 using 3-bromo-4-fluorobenzaldehyde and (3R,5S)-3-ethynyl-3-hydroxy-1-methyl-5-(trifluoromethyl)pyrrolidin-2-one (Intermediate 45) instead of 3-bromobenzaldehyde and (R)-3-ethynyl-3-hydroxy-1-methylpyrrolidin-2-one. MS (ESI): Mass calcd. for C₂₁H₂₃BF₄N₂O₅, 470.8; m/z found, 471.3 [M+H]⁺.

Step B: (3R,5S)-3-(3-(3-(4-Amino-8-methoxypyrido[3,2-d]pyrimidin-6-yl)-4-fluorophenyl)isoxazol-5-yl)-3-hydroxy-1-methyl-5-(trifluoromethyl)pyrrolidin-2-one. (3R,5S)-3-(3-(3-(4-Amino-8-methoxypyrido[3,2-d]pyrimidin-6-yl)-4-fluorophenyl)isoxazol-5-yl)-3-hydroxy-1-methyl-5-(trifluoromethyl)pyrrolidin-2-one (65 mg, 18%) was prepared in a manner analogous to Example 47, Step B using 6-chloro-8-methoxypyrido[3,2-d]pyrimidin-4-amine (Example 119 Step B) and (3R,5S)-3-(3-(4-fluoro-3-(4,4,5,5-tetramethyl-1,3,2-dioxaborolan-2-yl)phenyl)isoxazol-5-yl)-3-hydroxy-1-methyl-5-(trifluoromethyl)pyrrolidin-2-one instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₃H₁₈F₄N₆O₄, 518.1; m/z found, 519.2 [M+H]⁺. ¹H NMR (500 MHz, CD₃OD) δ 8.74 (dd, J=7.4, 2.3 Hz, 1H), 8.65 (s, 1H), 8.12-8.04 (m, 1H), 8.01 (s, 1H), 7.47 (dd, J=11.3, 8.6 Hz, 1H), 7.11 (s, 1H), 4.54-4.43 (m, 1H), 4.27 (s, 3H), 3.14-3.02 (m, 4H), 2.44 (dd, J=14.6, 5.0 Hz, 1H).

### Example 117: (R)-4-Cyclopropyl-6-(3-(5-(3-hydroxy-1-methyl-2-oxopyrrolidin-3-yl)isoxazol-3-yl)phenyl)picolinamide

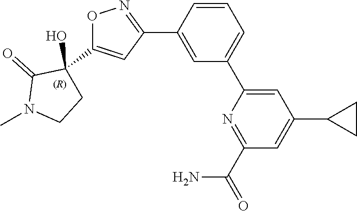

(R)-4-Cyclopropyl-6-(3-(5-(3-hydroxy-1-methyl-2-oxopyrrolidin-3-yl)isoxazol-3-yl)phenyl)picolinamide (90 mg) was prepared in a manner analogous to Example 88 using 4-bromo-6-chloropicolinonitrile instead of 3-amino-4,6-dichloropicolinonitrile in Step A. MS (ESI): Mass calcd. For C₂₃H₂₂N₄O₄, 418.2; m/z found, 419.1 [M+H]⁺. ¹H NMR (500 MHz, CD₃OD) δ 8.60 (t, J=1.8 Hz, 1H), 8.28-8.16 (m, 1H), 7.95-7.86 (m, 1H), 7.84 (d, J=1.6 Hz, 1H), 7.73 (d, J=1.5 Hz, 1H), 7.61 (t, J=7.8 Hz, 1H), 7.04 (s, 1H), 3.65-3.51 (m, 2H), 2.97 (s, 3H), 2.82-2.69 (m, 1H), 2.46-2.35 (m, 1H), 2.17-2.06 (m, 1H), 1.30-1.17 (m, 2H), 1.05-0.92 (m, 2H).

### Example 118: (R)-3-(3-(3-(4-Amino-8-methoxypyrido[3,2-d]pyrimidin-6-yl)phenyl)isoxazol-5-yl)-3-hydroxy-1-methylpyrrolidin-2-one

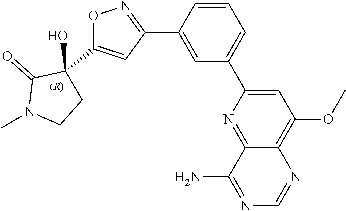

(R)-3-(3-(3-(4-Amino-8-methoxypyrido[3,2-d]pyrimidin-6-yl)phenyl)isoxazol-5-yl)-3-hydroxy-1-methylpyrrolidin-2-one (180 mg, 53%) was prepared in a manner analogous to Example 47, Step B using 6-chloro-8-methoxypyrido[3,2-d]pyrimidin-4-amine (Example 119 Step B) instead of 4-chloro-6-methoxypicolinamide. MS (ESI): Mass calcd. for C₂₂H₂₀N₆O₄, 432.2; m/z found, 433.3 [M+H]⁺. ¹H NMR (500 MHz, CD₃OD) δ 8.81 (s, 1H), 8.41 (d, J=14.0 Hz, 2H), 8.02-7.96 (m, 1H), 7.86 (d, J=1.2 Hz, 1H), 7.69 (t, J=7.8 Hz, 1H), 7.12 (s, 1H), 4.23 (s, 3H), 3.70-3.51 (m, 2H), 3.00 (s, 3H), 2.86-2.73 (m, 1H), 2.54-2.27 (m, 1H).

### Example 119: (R)-3-(3-(3-(4-Amino-8-methoxypyrido[3,2-d]pyrimidin-6-yl)-4-fluorophenyl)isoxazol-5-yl)-4,4-difluoro-3-hydroxy-1-methylpyrrolidin-2-one

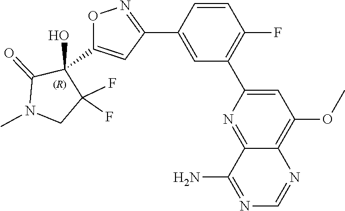

Step A: (R)-(5-(5-(4,4-Difluoro-3-hydroxy-1-methyl-2-oxopyrrolidin-3-yl)isoxazol-3-yl)-2-fluorophenyl)boronic acid. (R)-(5-(5-(4,4-Difluoro-3-hydroxy-1-methyl-2-oxopyrrolidin-3-yl)isoxazol-3-yl)-2-fluorophenyl)boronic acid (78 mg) was prepared in a manner analogous to Intermediate 3 and 4 using 3-bromo-4-fluorobenzaldehyde and (R)-3-ethynyl-4,4-difluoro-3-hydroxy-1-methylpyrrolidin-2-one (Intermediate 44) instead of 3-bromobenzaldehyde and (R)-3-ethynyl-3-hydroxy-1-methylpyrrolidin-2-one. MS (ESI): Mass calcd. for C₁₄H₁₂BF₃N₂O₅, 356.0; m/z found, 356.9 [M+H]⁺.

Step B: 6-Chloro-8-methoxypyrido[3,2-d]pyrimidin-4-amine. To 6,8-dichloropyrido[3,2-d]pyrimidin-4-amine (620 mg, 2.88 mmol) in DMA (2 mL), NaH (60% dispersion in mineral oil) (150 mg, 3.75 mmol) was added, followed by MeOH (0.234 mL, 5.76 mmol). The mixture was heated at 60° C. for 3 h. Water (30 mL) was added and the solids were formed. After filtration, the solid was collected and dried to provide the title compound (490 mg, 81%). MS (ESI): Mass calcd. for C₈H₇ClN₄O, 210.0; m/z found, 211.1 [M+H]⁺.

Step C: (R)-3-(3-(3-(4-Amino-8-methoxypyrido[3,2-d]pyrimidin-6-yl)-4-fluorophenyl)isoxazol-5-yl)-4,4-difluoro-3-hydroxy-1-methylpyrrolidin-2-one. (R)-3-(3-(3-(4-Amino-8-methoxypyrido[3,2-d]pyrimidin-6-yl)-4-fluorophenyl)isoxazol-5-yl)-4,4-difluoro-3-hydroxy-1-methylpyrrolidin-2-one (16 mg, 8.8%) was prepared in a manner analogous to Example 47 Step B using 6-chloro-8-methoxypyrido[3,2-d]pyrimidin-4-amine and (R)-(5-(5-(4,4-difluoro-3-hydroxy-1-methyl-2-oxopyrrolidin-3-yl)isoxazol-3-yl)-2-fluorophenyl)boronic acid instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₂H₁₇F₃N₆O₄, 486.1; m/z found, 487.2 [M+H]⁺. ¹H NMR (500 MHz, CD₃OD) δ 8.60 (dd, J=7.4, 2.4 Hz, 1H), 8.53 (s, 1H), 7.97-7.91 (m, 1H), 7.87 (s, 1H), 7.33 (dd, J=11.3, 8.6 Hz, 1H), 7.10 (s, 1H), 4.15 (s, 3H), 4.01-3.82 (m, 2H), 2.96 (s, 3H).

### Example 120: (R)-3-(3-(3-(4-Amino-8-methoxypyrido[3,2-d]pyrimidin-6-yl)-4-fluorophenyl)isoxazol-5-yl)-3-hydroxy-1-methylpyrrolidin-2-one

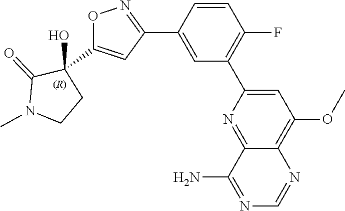

Step A: (R)-3-(3-(4-Fluoro-3-(4,4,5,5-tetramethyl-1,3,2-dioxaborolan-2-yl)phenyl)isoxazol-5-yl)-3-hydroxy-1-methylpyrrolidin-2-one. (R)-3-(3-(4-Fluoro-3-(4,4,5,5-tetramethyl-1,3,2-dioxaborolan-2-yl)phenyl)isoxazol-5-yl)-3-hydroxy-1-methylpyrrolidin-2-one (470 mg) was prepared in a manner analogous to Intermediate 3 and 4 using 3-bromo-4-fluorobenzaldehyde instead of 3-bromobenzaldehyde. MS (ESI): Mass calcd. for C₂₀H₂₄BFN₂O₅, 402.2; m/z found, 403.2 [M+H]⁺.

Step B: (R)-3-(3-(3-(4-Amino-8-methoxypyrido[3,2-d]pyrimidin-6-yl)-4-fluorophenyl)isoxazol-5-yl)-3-hydroxy-1-methylpyrrolidin-2-one. (R)-3-(3-(3-(4-Amino-8-methoxypyrido[3,2-d]pyrimidin-6-yl)-4-fluorophenyl)isoxazol-5-yl)-3-hydroxy-1-methylpyrrolidin-2-one (26 mg, 18%) was prepared in a manner analogous to Example 47 Step A using 6-chloro-8-methoxypyrido[3,2-d]pyrimidin-4-amine (Example 119 Step B) and (R)-3-(3-(4-fluoro-3-(4,4,5,5-tetramethyl-1,3,2-dioxaborolan-2-yl)phenyl)isoxazol-5-yl)-3-hydroxy-1-methylpyrrolidin-2-one instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₂H₁₉FN₆O₄, 450.1; m/z found, 451.3 [M+H]⁺. ¹H NMR (500 MHz, CD₃OD) δ 8.69 (dd, J=7.4, 2.4 Hz, 1H), 8.42 (s, 1H), 8.08-7.95 (m, 1H), 7.77 (s, 1H), 7.45 (dd, J=11.2, 8.5 Hz, 1H), 7.06 (s, 1H), 4.17 (s, 3H), 3.65-3.56 (m, 2H), 2.99 (s, 3H), 2.82-2.71 (m, 1H), 2.50-2.34 (m, 1H).

### Example 121: (R)-2-(2-Fluoro-5-(5-(3-hydroxy-1-methyl-2-oxopyrrolidin-3-yl)isoxazol-3-yl)phenyl)-5-(oxetan-3-ylamino)pyrimidine-4-carboxamide

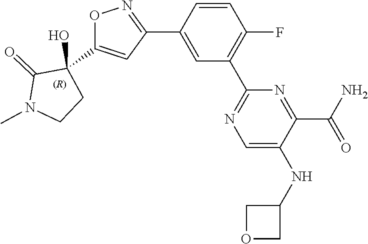

Step A: (R)-3-(3-(4-Fluoro-3-(4,4,5,5-tetramethyl-1,3,2-dioxaborolan-2-yl)phenyl)isoxazol-5-yl)-3-hydroxy-1-methylpyrrolidin-2-one (470 mg) was prepared in a manner analogous to Intermediate 3 and 4 using 3-bromo-4-fluorobenzaldehyde instead of 3-bromobenzaldehyde. MS (ESI): Mass calcd. for C₂₀H₂₄BFN₂O₅, 402.2; m/z found, 403.2 [M+H]⁺.

Step B: (R)-2-(2-Fluoro-5-(5-(3-hydroxy-1-methyl-2-oxopyrrolidin-3-yl)isoxazol-3-yl)phenyl)-5-(oxetan-3-ylamino)pyrimidine-4-carboxamide. (R)-2-(2-Fluoro-5-(5-(3-hydroxy-1-methyl-2-oxopyrrolidin-3-yl)isoxazol-3-yl)phenyl)-5-(oxetan-3-ylamino)pyrimidine-4-carboxamide (7 mg, 3%) was prepared in a manner analogous to Example 105 using (R)-3-(3-(4-fluoro-3-(4,4,5,5-tetramethyl-1,3,2-dioxaborolan-2-yl)phenyl)isoxazol-5-yl)-3-hydroxy-1-methylpyrrolidin-2-one (Example 120, Step A) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₂H₂₁FN₆O₅, 468.2; m/z found, 469.1 [M+H]⁺. ¹H NMR (500 MHz, Chloroform-d) δ 8.44 (d, J=6.2 Hz, 1H), 8.25 (dd, J=7.4, 2.4 Hz, 1H), 8.10 (s, 1H), 8.03 (d, J=4.4 Hz, 1H), 7.67-7.62 (m, 1H), 7.07 (dd, J=10.9, 8.6 Hz, 1H), 6.62 (s, 1H), 5.78 (d, J=4.0 Hz, 1H), 4.96 (t, J=6.7 Hz, 2H), 4.80-4.69 (m, 1H), 4.61 (t, J=6.1 Hz, 2H), 3.62-3.50 (m, 1H), 3.47-3.37 (m, 1H), 2.92 (s, 3H), 2.72-2.59 (m, 1H), 2.46-2.31 (m, 1H).

### Example 122: (R)-1-Cyclopropyl-4-(3-(5-(3-hydroxy-1-methyl-2-oxopyrrolidin-3-yl)isoxazol-3-yl)phenyl)-5-methyl-1H-imidazole-2-carboxamide

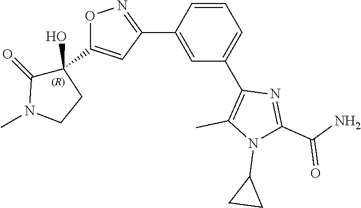

Step A: Ethyl 4-bromo-1-cyclopropyl-5-methyl-1H-imidazole-2-carboxylate. Ethyl 4-bromo-1-cyclopropyl-5-methyl-1H-imidazole-2-carboxylate (35 mg, 13%) was prepared in a manner analogous to Example 95 Step A using ethyl 4-bromo-5-methyl-1H-imidazole-2-carboxylate and cyclopropylboronic acid instead of methyl 4,5,6,7-tetrahydro-1H-indazole-3-carboxylate and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₁₀H₁₃BrN₂O₂, 272.0; m/z found, 273.1 [M+H]⁺.

Step B: (R)-1-Cyclopropyl-4-(3-(5-(3-hydroxy-1-methyl-2-oxopyrrolidin-3-yl)isoxazol-3-yl)phenyl)-5-methyl-1H-imidazole-2-carboxamide. (R)-1-Cyclopropyl-4-(3-(5-(3-hydroxy-1-methyl-2-oxopyrrolidin-3-yl)isoxazol-3-yl)phenyl)-5-methyl-1H-imidazole-2-carboxamide (7 mg) was prepared in a manner analogous to Example 109 using ethyl 4-bromo-1-cyclopropyl-5-methyl-1H-imidazole-2-carboxylate instead of ethyl 1-bromoimidazo[1,5-a]pyrazine-3-carboxylate. MS (ESI): Mass calcd. for C₂₂H₂₃N₅O₄, 421.2; m/z found, 422.2 [M+H]⁺. ¹H NMR (500 MHz, CD₃OD) δ 8.18-8.04 (m, 1H), 7.97-7.87 (m, 1H), 7.79-7.71 (m, 1H), 7.66 (t, J=7.8 Hz, 1H), 6.96 (s, 1H), 3.68-3.48 (m, 3H), 2.98 (s, 3H), 2.83-2.70 (m, 1H), 2.61 (s, 3H), 2.48-2.38 (m, 1H), 1.39-1.30 (m, 2H), 1.14-1.06 (m, 2H).

### Example 123: (R)-3-(3-(3-(8-amino-4-methylpyrimido[5,4-d]pyrimidin-2-yl)-4,5-difluorophenyl)isoxazol-5-yl)-4,4-difluoro-3-hydroxy-1-methylpyrrolidin-2-one

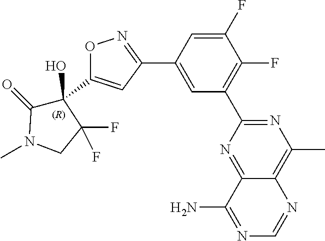

Step A: (R)-(5-(5-(4,4-Difluoro-3-hydroxy-1-methyl-2-oxopyrrolidin-3-yl)isoxazol-3-yl)-2,3-difluorophenyl)boronic acid. (R)-(5-(5-(4,4-Difluoro-3-hydroxy-1-methyl-2-oxopyrrolidin-3-yl)isoxazol-3-yl)-2,3-difluorophenyl)boronic acid (610 mg) was prepared in a manner analogous to Intermediate 3 and 4 using 3-bromo-4,5-difluorobenzaldehyde and (R)-3-ethynyl-4,4-difluoro-3-hydroxy-1-methylpyrrolidin-2-one (Intermediate 44) instead of 3-bromobenzaldehyde and (R)-3-ethynyl-3-hydroxy-1-methylpyrrolidin-2-one. MS (ESI): Mass calcd. for C₁₄H₁₁BF₄N₂O₅, 374.1; m/z found, 375.1 [M+H]⁺.

Step B: (R)-3-(3-(3-(8-Amino-4-methylpyrimido[5,4-d]pyrimidin-2-yl)-4,5-difluorophenyl)isoxazol-5-yl)-4,4-difluoro-3-hydroxy-1-methylpyrrolidin-2-one. (R)-3-(3-(3-(8-Amino-4-methylpyrimido[5,4-d]pyrimidin-2-yl)-4,5-difluorophenyl)isoxazol-5-yl)-4,4-difluoro-3-hydroxy-1-methylpyrrolidin-2-one (16 mg, 8.8%) was prepared in a manner analogous to Example 47 Step A using 6-chloro-8-methylpyrimido[5,4-d]pyrimidin-4-amine and (R)-(5-(5-(4,4-difluoro-3-hydroxy-1-methyl-2-oxopyrrolidin-3-yl)isoxazol-3-yl)-2,3-difluorophenyl)boronic acid instead of 4-chloro-6-methoxypicolinamide and (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₁H₁₅F₄N₇O₃, 489.1; m/z found, 490.3 [M+H]⁺. ¹H NMR (500 MHz, CD₃OD) δ 8.61-8.51 (m, 2H), 8.04-7.94 (m, 1H), 7.22 (s, 1H), 4.13-3.90 (m, 2H), 3.07 (s, 3H), 2.99 (s, 3H).

### Example 124: (R)-3-(3-(5-(3-Hydroxy-1-methyl-2-oxopyrrolidin-3-yl)isoxazol-3-yl)phenyl)-5,6,7,8-tetrahydroisoquinoline-1-carboxamide

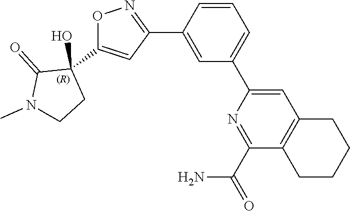

Step A: 3-Chloro-5,6,7,8-tetrahydroisoquinoline 2-oxide. The mixture of 3-chloro-5,6,7,8-tetrahydroisoquinoline (503 mg, 3.00 mmol) with 3-chloroperoxybenzoic acid (1.03 g, 6.00 mmol) in dichloromethane (20 mL) was stirred at room temperature for 4 h. The mixture was then washed saturated aqueous NaHCO₃ solution (20 mL). The organic layer was separated and concentrated. The residue was purified by FCC (DCM/MeOH) providing the title compound (360 mg, 65.3%). MS (ESI): Mass calcd. for C₉H₁₀ClNO, 183.0; m/z found, 184.2 [M+H]⁺.

Step B: 3-Chloro-5,6,7,8-tetrahydroisoquinoline-1-carbonitrile. Trimethylsilyl cyanide (389 mg, 3.92 mmol) was added to a stirred solution of 3-chloro-5,6,7,8-tetrahydroisoquinoline 2-oxide (360 mg, 1.96 mmol) and Et₃N (0.817 mL, 5.88 mmol) in CH₃CN (25 mL) at room temperature. The mixture was then heated at 110° C. for 12 h, cooled down to room temperature, and the solvent was evaporated. DCM (25 mL) and saturated aqueous NaHCO₃ solution (15 mL) were added. The organic layers were separated and concentrated. The residue was washed with ether and filtered, then evaporated to give the product (201 mg, 52.2%). MS (ESI): Mass calcd. for C₁₀H₁₉ClN₂, 192.1; m/z found, 193.1 [M+H]⁺.

Step C: (R)-3-(3-(5-(3-Hydroxy-1-methyl-2-oxopyrrolidin-3-yl)isoxazol-3-yl)phenyl)-5,6,7,8-tetrahydroisoquinoline-1-carboxamide. (R)-3-(3-(5-(3-Hydroxy-1-methyl-2-oxopyrrolidin-3-yl)isoxazol-3-yl)phenyl)-5,6,7,8-tetrahydroisoquinoline-1-carboxamide (150 mg) was prepared in a manner analogous to Example 90 (Step B and Step C) using 3-chloro-5,6,7,8-tetrahydroisoquinoline-1-carbonitrile instead of 6-chloro-1-ethyl-1H-imidazo[4,5-c]pyridine-4-carbonitrile. MS (ESI): Mass calcd. for C₂₄H₂₄N₄O₄, 432.2; m/z found, 433.2 [M+H]⁺. ¹H NMR (500 MHz, CD₃OD) δ 8.39 (t, J=1.8 Hz, 1H), 8.08-7.98 (m, 1H), 7.89-7.83 (m, 1H), 7.77 (s, 1H), 7.61-7.42 (m, 2H), 6.92 (s, 1H), 3.59-3.43 (m, 2H), 3.08-2.99 (m, 2H), 2.88 (s, 5H), 2.73-2.62 (m, 1H), 2.37-2.27 (m, 1H), 1.87-1.69 (m, 4H).

### Example 125: (R)-2-(3-(5-(3-Hydroxy-1-methyl-2-oxopyrrolidin-3-yl)isoxazol-3-yl)phenyl)-5,6,7,8-tetrahydroquinazoline-4-carboxamide

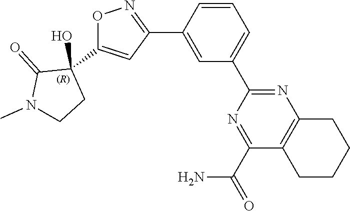

Step A: (R)-3-(5-(3-Hydroxy-1-methyl-2-oxopyrrolidin-3-yl)isoxazol-3-yl)benzonitrile. To a solution of 3-cyano-N-hydroxybenzimidoyl chloride (541 mg, 3.00 mmol) in DCM (20 mL) was added (R)-3-ethynyl-3-hydroxy-1-methylpyrrolidin-2-one (Intermediate 1, 417 mg, 3.00 mmol) followed by TEA (1.25 mL, 9.00 mmol). The reaction mixture was stirred at 25° C. for 3.5 h and concentrated. The residue was diluted with H₂O (100 mL). Then the solids were formed, filtered and dried providing the title compound (610 mg, 72%). MS (ESI): Mass calcd. for C₁₅H₁₃N₃O₃, 283.3; m/z found, 284.3 [M+H]⁺.

Step B: (R)-3-(5-(3-Hydroxy-1-methyl-2-oxopyrrolidin-3-yl)isoxazol-3-yl)benzimidamide. LiHMDS (1.75 mL, 1.75 mmol, 1 N-solution in n-hexanes) was added to (R)-3-(5-(3-hydroxy-1-methyl-2-oxopyrrolidin-3-yl)isoxazol-3-yl)benzonitrile (141 mg, 0.500 mmol) in THF (10 mL). The reaction was stirred for 2 h at rt. The mixture was concentrated to dryness to provide the title compound which was used as it is for the next step. MS (ESI): Mass calcd. for C₁₅H₁₆N₄O₃, 300.1; m/z found, 301.2 [M+H]⁺.

Step C: Ethyl (R)-2-(3-(5-(3-hydroxy-1-methyl-2-oxopyrrolidin-3-yl)isoxazol-3-yl)phenyl)-5,6,7,8-tetrahydroquinazoline-4-carboxylate. The mixture of (R)-3-(5-(3-hydroxy-1-methyl-2-oxopyrrolidin-3-yl)isoxazol-3-yl)benzimidamide (150 mg, 0.50 mmol), ethyl 2-oxo-2-(2-oxocyclohexyl)acetate (99 mg, 0.50 mmol) and Et₃N (0.209 mL, 1.50 mmol) in CH₃CN (10 mL) was heated at 130° C. for 16 h. The solids were filtered out and the filtrate was concentrated. The residue was re-dissolved into DMSO (2 mL) and purified with HPLC (CH₃CN/H₂O) to provide the title compound (19 mg, 8.2%). MS (ESI): Mass calcd. for C₂₅H₂₆N₄O₅, 462.2; m/z found, 463.3 [M+H]⁺.

Step D: (R)-2-(3-(5-(3-Hydroxy-1-methyl-2-oxopyrrolidin-3-yl)isoxazol-3-yl)phenyl)-5,6,7,8-tetrahydroquinazoline-4-carboxamide. (R)-2-(3-(5-(3-Hydroxy-1-methyl-2-oxopyrrolidin-3-yl)isoxazol-3-yl)phenyl)-5,6,7,8-tetrahydroquinazoline-4-carboxamide (4.0 mg, 22%) was prepared in a manner analogous to Example 47 Step A using ethyl (R)-2-(3-(5-(3-hydroxy-1-methyl-2-oxopyrrolidin-3-yl)isoxazol-3-yl)phenyl)-5,6,7,8-tetrahydroquinazoline-4-carboxylate instead of methyl 4-chloro-6-methoxypicolinate. MS (ESI): Mass calcd. for C₂₃H₂₃N₅O₄, 433.2; m/z found, 434.2 [M+H]⁺. ¹H NMR (400 MHz, Chloroform-d) δ 8.82 (t, J=1.7 Hz, 1H), 8.58-8.40 (m, 1H), 7.97-7.85 (m, 1H), 7.58 (t, J=7.8 Hz, 1H), 6.84 (s, 1H), 3.73-3.58 (m, 1H), 3.58-3.48 (m, 1H), 3.33 (t, J=6.3 Hz, 2H), 3.12-2.99 (m, 5H), 2.87-2.74 (m, 1H), 2.57-2.43 (m, 1H), 1.98-1.85 (m, 4H).

### Example 126: 5-Amino-2-(3-(5-((R)-3-hydroxy-1-methyl-2-oxopyrrolidin-3-yl)isoxazol-3-yl)phenyl)-N—((S)-tetrahydrofuran-3-yl)pyrimidine-4-carboxamide

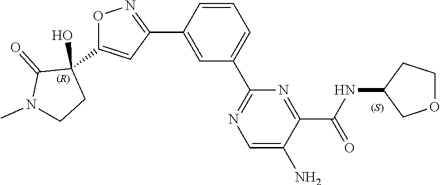

5-Amino-2-(3-(5-((R)-3-hydroxy-1-methyl-2-oxopyrrolidin-3-yl)isoxazol-3-yl)phenyl)-N—((S)-tetrahydrofuran-3-yl)pyrimidine-4-carboxamide (19 mg) was prepared as one of the two products in a manner analogous to Example 105 using (S)-tetrahydrofuran-3-amine instead of 3-oxetanamine. The other product is described as Example 127. MS (ESI): Mass calcd. for C₂₃H₂₄N₆O₅, 464.2; m/z found, 465.3 [M+H]⁺. ¹H NMR (600 MHz, CD₃OD) δ 8.79 (s, 1H), 8.53 (s, 1H), 8.44 (d, J=7.9 Hz, 1H), 7.93-7.81 (m, 1H), 7.58 (t, J=7.8 Hz, 1H), 6.99 (s, 1H), 4.66-4.61 (m, 2H), 4.12-4.04 (m, 1H), 4.04-3.94 (m, 1H), 3.92-3.85 (m, 1H), 3.85-3.77 (m, 1H), 3.62-3.52 (m, 2H), 2.97 (s, 3H), 2.81-2.65 (m, 1H), 2.49-2.35 (m, 2H), 2.19-2.08 (m, 1H).

### Example 127: 2-(3-(5-((R)-3-hydroxy-1-methyl-2-oxopyrrolidin-3-yl)isoxazol-3-yl)phenyl)-5-(((S)-tetrahydrofuran-3-yl)amino)pyrimidine-4-carboxamide

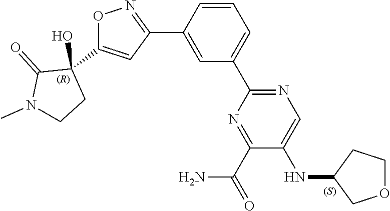

2-(3-(5-((R)-3-Hydroxy-1-methyl-2-oxopyrrolidin-3-yl)isoxazol-3-yl)phenyl)-5-(((S)-tetrahydrofuran-3-yl)amino)pyrimidine-4-carboxamide (62 mg) was prepared as one of the two products in a manner analogous to Example 105 using (S)-tetrahydrofuran-3-amine instead of 3-oxetanamine. The other product is described as Example 126. MS (ESI): Mass calcd. for C₂₃H₂₄N₆O₅, 464.2; m/z found, 465.2 [M+H]⁺. ¹H NMR (500 MHz, CD₃OD) δ 8.82 (t, J=1.8 Hz, 1H), 8.62 (s, 1H), 8.50-8.42 (m, 1H), 7.91-7.83 (m, 1H), 7.58 (t, J=7.8 Hz, 1H), 7.01 (s, 1H), 4.45-4.36 (m, 1H), 4.13-3.95 (m, 2H), 3.95-3.88 (m, 1H), 3.76 (dd, J=9.2, 3.0 Hz, 1H), 3.62-3.54 (m, 2H), 2.97 (s, 3H), 2.82-2.72 (m, 1H), 2.48-2.36 (m, 2H), 2.03-1.90 (m, 1H).

### Example 128: 5-Amino-2-(3-(5-((R)-3-hydroxy-1-methyl-2-oxopyrrolidin-3-yl)isoxazol-3-yl)phenyl)-N—((R)-tetrahydrofuran-3-yl)pyrimidine-4-carboxamide

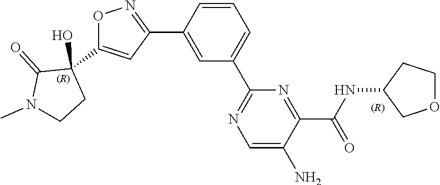

5-Amino-2-(3-(5-((R)-3-hydroxy-1-methyl-2-oxopyrrolidin-3-yl)isoxazol-3-yl)phenyl)-N—((R)-tetrahydrofuran-3-yl)pyrimidine-4-carboxamide (15 mg) was prepared as one of the two products in a manner analogous to Example 105 using (R)-tetrahydrofuran-3-amine instead of 3-oxetanamine. The other product is described as Example 129. MS (ESI): Mass calcd. for C₂₃H₂₄N₆O₅, 464.2; m/z found, 465.3 [M+H]⁺. ¹H NMR (600 MHz, CD₃OD) d 8.79 (s, 1H), 8.53 (s, 1H), 8.44 (d, J=7.9 Hz, 1H), 7.93-7.81 (m, 1H), 7.58 (t, J=7.8 Hz, 1H), 6.99 (s, 1H), 4.66-4.61 (m, 2H), 4.12-4.04 (m, 1H), 4.04-3.94 (m, 1H), 3.92-3.85 (m, 1H), 3.85-3.77 (m, 1H), 3.62-3.52 (m, 2H), 2.97 (s, 3H), 2.81-2.65 (m, 1H), 2.49-2.35 (m, 2H), 2.19-2.08 (m, 1H).

### Example 129: 2-(3-(5-((R)-3-hydroxy-1-methyl-2-oxopyrrolidin-3-yl)isoxazol-3-yl)phenyl)-5-(((R)-tetrahydrofuran-3-yl)amino)pyrimidine-4-carboxamide

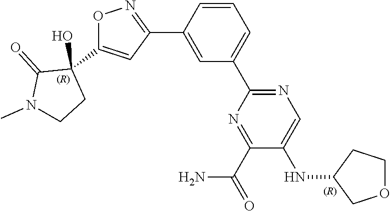

2-(3-(5-((R)-3-Hydroxy-1-methyl-2-oxopyrrolidin-3-yl)isoxazol-3-yl)phenyl)-5-(((R)-tetrahydrofuran-3-yl)amino)pyrimidine-4-carboxamide (105 mg) was prepared in a manner analogous to Example 105 using (R)-tetrahydrofuran-3-amine instead of 3-oxetanamine. The other product is described as Example 128. MS (ESI): Mass calcd. for C₂₃H₂₄N₆O₅, 464.2; m/z found, 465.2 [M+H]⁺. ¹H NMR (500 MHz, CD₃OD) δ 8.82 (t, J=1.8 Hz, 1H), 8.62 (s, 1H), 8.52-8.44 (m, 1H), 7.91-7.84 (m, 1H), 7.58 (t, J=7.8 Hz, 1H), 7.01 (s, 1H), 4.44-4.36 (m, 1H), 4.09-3.97 (m, 2H), 3.95-3.87 (m, 1H), 3.76 (dd, J=9.2, 3.0 Hz, 1H), 3.63-3.53 (m, 2H), 2.97 (s, 3H), 2.81-2.74 (m, 1H), 2.48-2.36 (m, 2H), 2.03-1.85 (m, 1H).

### Example 130: (R)-2-(3-(5-(3-Hydroxy-1-methyl-2-oxopyrrolidin-3-yl)isoxazol-3-yl)phenyl)-5-((1-methyl-1H-pyrazol-3-yl)amino)pyrimidine-4-carboxamide

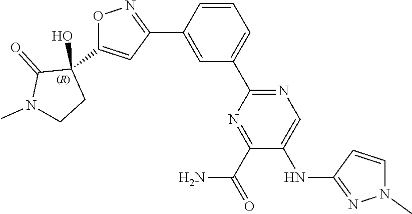

Step A: Ethyl 2-chloro-5-((1-methyl-H-pyrazol-3-yl)amino)pyrimidine-4-carboxylate. Ethyl 2-chloro-5-((1-methyl-1H-pyrazol-3-yl)amino)pyrimidine-4-carboxylate (1.2 g, 98%) was prepared in a manner analogous to Example 105 Step A using 1-methyl-1H-pyrazol-3-ylamine instead of 3-oxetanamine. MS (ESI): Mass calcd. for C₁₁H₁₂ClN₅O₂, 281.1; m/z found, 282.2 [M+H]⁺.

Step B: (R)-2-(3-(5-(3-Hydroxy-1-methyl-2-oxopyrrolidin-3-yl)isoxazol-3-yl)phenyl)-5-(oxetan-3-ylamino)pyrimidine-4-carboxamide. (R)-2-(3-(5-(3-Hydroxy-1-methyl-2-oxopyrrolidin-3-yl)isoxazol-3-yl)phenyl)-5-(oxetan-3-ylamino)pyrimidine-4-carboxamide (25 mg) was prepared in a manner analogous to Example 109 using ethyl 2-chloro-5-((1-methyl-1H-pyrazol-3-yl)amino)pyrimidine-4-carboxylate instead of ethyl 1-bromoimidazo[1,5-a]pyrazine-3-carboxylate. MS (ESI): Mass calcd. for C₂₃H₂₂N₈O₄, 474.2; m/z found, 475.4 [M+H]⁺. ¹H NMR (600 MHz, CD₃OD) δ 9.67 (s, 1H), 8.87 (s, 1H), 8.52 (d, J=7.9 Hz, 1H), 7.90 (d, J=7.6 Hz, 1H), 7.60 (t, J=7.7 Hz, 1H), 7.52 (d, J=2.3 Hz, 1H), 7.02 (s, 1H), 6.05 (d, J=2.3 Hz, 1H), 3.87 (s, 3H), 3.65-3.51 (m, 2H), 2.97 (s, 3H), 2.83-2.65 (m, 1H), 2.46-2.36 (m, 1H).

### Example 131: 6-(3-(5-((R)-3-Hydroxy-1-methyl-2-oxopyrrolidin-3-yl)isoxazol-3-yl)phenyl)-4-(((S)-tetrahydrofuran-3-yl)amino)picolinamide

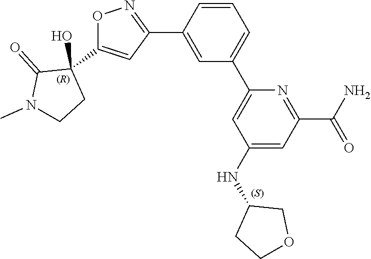

Step A: (S)-6-Chloro-4-((tetrahydrofuran-3-yl)amino)picolinonitrile. (S)-6-Chloro-4-((tetrahydrofuran-3-yl)amino)picolinonitrile (450 mg, 100%) was prepared in a manner analogous to Example 105 Step A using (S)-tetrahydrofuran-3-amine and 4-bromo-6-chloropicolinonitrile instead of 3-oxetanamine and ethyl 2-chloro-5-fluoropyrimidine-4-carboxylate. MS (ESI): Mass calcd. for C₁₀H₁₀ClN₃O₂, 223.1; m/z found, 224.2 [M+H]⁺.

Step B: 6-(3-(5-((R)-3-Hydroxy-1-methyl-2-oxopyrrolidin-3-yl)isoxazol-3-yl)phenyl)-4-(((S)-tetrahydrofuran-3-yl)amino)picolinamide. 6-(3-(5-((R)-3-Hydroxy-1-methyl-2-oxopyrrolidin-3-yl)isoxazol-3-yl)phenyl)-4-(((S)-tetrahydrofuran-3-yl)amino)picolinamide (26 mg) was prepared in a manner analogous to Example 90 (Step B and Step C) using (S)-6-chloro-4-((tetrahydrofuran-3-yl)amino)picolinonitrile instead of 6-chloro-1-ethyl-1H-imidazo[4,5-c]pyridine-4-carbonitrile. MS (ESI): Mass calcd. for C₂₄H₂₅N₅O₅, 463.2; m/z found, 464.3 [M+H]⁺. ¹H NMR (600 MHz, CD₃OD) δ 8.17-8.08 (m, 1H), 7.90 (d, J=9.9 Hz, 1H), 7.79 (d, J=12.5 Hz, 1H), 7.68-7.55 (m, 2H), 7.02-6.91 (m, 2H), 4.68-4.60 (m, 1H), 4.11-4.04 (m, 1H), 4.04-3.92 (m, 1H), 3.92-3.85 (m, 1H), 3.74-3.67 (m, 1H), 3.62-3.54 (m, 2H), 2.96 (s, 3H), 2.78-2.69 (m, 1H), 2.47-2.32 (m, 2H), 1.99-1.90 (m, 1H).

### Example 132: (R)-5-Amino-6-cyclopropyl-2-(3-(5-(3-hydroxy-1-methyl-2-oxopyrrolidin-3-yl)isoxazol-3-yl)phenyl)pyrimidine-4-carboxamide

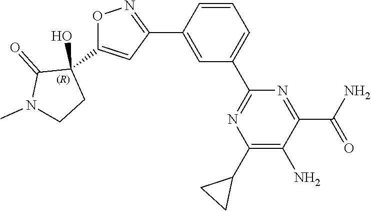

Step A: Methyl 5-amino-2-chloro-6-cyclopropylpyrimidine-4-carboxylate. Methyl 5-amino-2-chloro-6-cyclopropylpyrimidine-4-carboxylate (87 mg, 38%) was prepared in a manner analogous to Example 88 using methyl 5-amino-2,6-dichloropyrimidine-4-carboxylate instead of 3-amino-4,6-dichloropicolinonitrile. MS (ESI): Mass calcd. for C₉H₁₀N₃O₂, 227.0; m/z found, 228.1 [M+H]⁺.

Step B: (R)-5-Amino-6-cyclopropyl-2-(3-(5-(3-hydroxy-1-methyl-2-oxopyrrolidin-3-yl)isoxazol-3-yl)phenyl)pyrimidine-4-carboxamide. (R)-5-Amino-6-cyclopropyl-2-(3-(5-(3-hydroxy-1-methyl-2-oxopyrrolidin-3-yl)isoxazol-3-yl)phenyl)pyrimidine-4-carboxamide (30 mg) was prepared in a manner analogous to Example 109 using methyl 5-amino-2-chloro-6-cyclopropylpyrimidine-4-carboxylate instead of ethyl 1-bromoimidazo[1,5-a]pyrazine-3-carboxylate. MS (ESI): Mass calcd. for C₂₂H₂₂N₆O₄, 434.2; m/z found, 435.2 [M+H]⁺. ¹H NMR (600 MHz, CD₃OD) δ 8.78 (t, J=1.8 Hz, 1H), 8.43 (d, J=7.9 Hz, 1H), 7.91-7.78 (m, 1H), 7.54 (t, J=7.8 Hz, 1H), 6.98 (s, 1H), 3.66-3.52 (m, 2H), 2.97 (s, 3H), 2.83-2.71 (m, 1H), 2.48-2.38 (m, 1H), 2.35-2.26 (m, 1H), 1.34-1.22 (m, 2H), 1.22-1.08 (m, 2H).

### Example 133: (R)-2-(3-(5-(3-Hydroxy-1-methyl-2-oxopyrrolidin-3-yl)isoxazol-3-yl)phenyl)-6-((1-methyl-1H-pyrazol-3-yl)amino)-5-(trifluoromethyl)pyrimidine-4-carboxamide

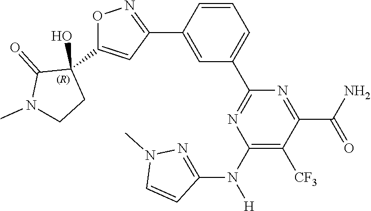

(R)-2-(3-(5-(3-Hydroxy-1-methyl-2-oxopyrrolidin-3-yl)isoxazol-3-yl)phenyl)-6-((1-methyl-1H-pyrazol-3-yl)amino)-5-(trifluoromethyl)pyrimidine-4-carboxamide (40 mg) was prepared in a manner analogous to Example 122 using methyl 2,6-dichloro-5-(trifluoromethyl)-4-pyrimidinecarboxylate instead of ethyl 2-chloro-5-fluoropyrimidine-4-carboxylate. MS (ESI): Mass calcd. for C₂₄H₂₁F₃N₈O₄, 542.2; m/z found, 543.1 [M+H]⁺. ¹H NMR (600 MHz, CD₃OD) δ 8.85 (s, 1H), 8.49 (d, J=8.0 Hz, 1H), 8.02 (d, J=7.6 Hz, 1H), 7.70 (d, J=2.5 Hz, 1H), 7.62 (t, J=7.8 Hz, 1H), 6.91 (d, J=7.6 Hz, 1H), 6.72 (d, J=2.4 Hz, 1H), 3.91 (s, 3H), 3.64-3.53 (m, 2H), 2.97 (s, 3H), 2.78-2.71 (m, 1H), 2.45-2.38 (s, 1H).

### Example 134: (R)-2-(3-(5-(3-Hydroxy-1-methyl-2-oxopyrrolidin-3-yl)isoxazol-3-yl)phenyl)-5H-pyrrolo[3,2-d]pyrimidine-4-carboxamide

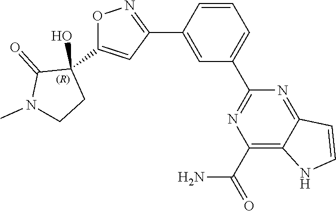

Step A: Methyl 2-chloro-5-tosyl-5H-pyrrolo[3,2-d]pyrimidine-4-carboxylate. To a mixture of methyl 2-chloro-5H-pyrrolo[3,2-d]pyrimidine-4-carboxylate (211 mg, 0.997 mmol), p-toluenesulfonyl chloride (215 mg, 1.12 mmol), and DMAP (12 mg, 0.010 mmol) in DCM (10 mL) was added Et₃N (0.21 mL, 1.5 mmol). The mixture was heated at 50° C. for 16 h. Then it was cooled down and diluted with saturated aqueous NaHCO₃ solution (5 mL). The organic layer was separated and concentrated providing the desired product which was used as it is for the next step (301 mg, 82.1%). MS (ESI): Mass calcd. For C₁₅H₁₂ClN₃O₄S, 365.0; m/z found, 366.1 [M+H]⁺.

Step B: (R)-2-(3-(5-(3-Hydroxy-1-methyl-2-oxopyrrolidin-3-yl)isoxazol-3-yl)phenyl)-5-tosyl-5H-pyrrolo[3,2-d]pyrimidine-4-carboxylate. (R)-2-(3-(5-(3-Hydroxy-1-methyl-2-oxopyrrolidin-3-yl)isoxazol-3-yl)phenyl)-5-tosyl-5H-pyrrolo[3,2-d]pyrimidine-4-carboxylate (210 mg, 44%) was prepared in a manner analogous to Example 47 Step B using methyl 2-chloro-5-tosyl-5H-pyrrolo[3,2-d]pyrimidine-4-carboxylate instead of 4-chloro-6-methoxypicolinamide. MS (ESI): Mass calcd. for C₂₉H₂₅N₅O₇S, 418.1; m/z found, 419.3 [M+H]⁺.

Step C: (R)-2-(3-(5-(3-hydroxy-1-methyl-2-oxopyrrolidin-3-yl)isoxazol-3-yl)phenyl)-5H-pyrrolo[3,2-d]pyrimidine-4-carboxamide. A mixture of methyl (R)-2-(3-(5-(3-hydroxy-1-methyl-2-oxopyrrolidin-3-yl)isoxazol-3-yl)phenyl)-5-tosyl-5H-pyrrolo[3,2-d]pyrimidine-4-carboxylate (210 mg, 0.36 mmol) in NH₃ (1.79 mL, 7 M in MeOH) was heated at 120° C. for 16 h. The reaction mixture was concentrated and the residue was purified with reverse phase HPLC to provide the desired product (22 mg, 15%). MS (ESI): Mass calcd. for C₂₁H₁₈N₆O₄, 418.1; m/z found, 419.3 [M+H]⁺. ¹H NMR (400 MHz, CD₃OD) δ 9.03 (t, J=1.7 Hz, 1H), 8.75-8.62 (m, 1H), 8.04-7.92 (m, 2H), 7.67 (t, J=7.8 Hz, 1H), 7.08 (s, 1H), 6.81 (d, J=3.2 Hz, 1H), 3.69-3.56 (m, 2H), 3.00 (s, 3H), 2.89-2.73 (m, 1H), 2.54-2.36 (m, 1H).

### Example 135: (R)-5-Amino-2-(3-(5-(3-Hydroxy-1-methyl-2-oxopyrrolidin-3-yl)isoxazol-3-yl)phenyl)pyrimidine-4-carboxamide

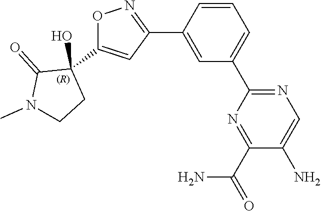

(R)-5-Amino-2-(3-(5-(3-Hydroxy-1-methyl-2-oxopyrrolidin-3-yl)isoxazol-3-yl)phenyl)pyrimidine-4-carboxamide (30 mg) was prepared in a manner analogous to Example 109 using ethyl 5-amino-2-chloropyrimidine-4-carboxylate instead of ethyl 1-bromoimidazo[1,5-a]pyrazine-3-carboxylate. MS (ESI): Mass calcd. for C₁₉H₁₈N₆O₄, 394.1; m/z found, 395.3 [M+H]⁺. ¹H NMR (500 MHz, CD₃OD) δ 8.86-8.68 (m, 1H), 8.53 (s, 1H), 8.50-8.37 (m, 1H), 7.91-7.82 (m, 1H), 7.65-7.52 (m, 1H), 7.02 (s, 1H), 3.66-3.54 (m, 2H), 2.99 (s, 3H), 2.84-2.70 (m, 1H), 2.53-2.34 (m, 1H).

### Example 136: (R)-3-(3-(3-(5-Amino-4,6-dimethoxypyrimidin-2-yl)phenyl)isoxazol-5-yl)-3-hydroxy-1-methylpyrrolidin-2-one

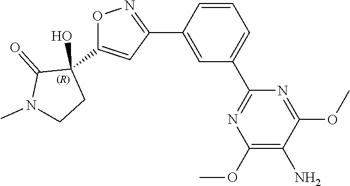

(R)-3-(3-(3-(5-Amino-4,6-dimethoxypyrimidin-2-yl)phenyl)isoxazol-5-yl)-3-hydroxy-1-methylpyrrolidin-2-one (42 mg) was prepared in a manner analogous to Example 47 using 2-chloro-4,6-dimethoxy-5-pyrimidinamine instead of methyl 4-chloro-6-methoxypicolinate in Step A. MS (ESI): Mass calcd. for C₂₀H₂₁N₅O₅, 411.2; m/z found, 412.1 [M+H]⁺. ¹H NMR (600 MHz, CD₃OD) δ 8.81 (t, J=1.8 Hz, 1H), 8.54-8.40 (m, 1H), 7.96-7.83 (m, 1H), 7.57 (t, J=7.7 Hz, 1H), 6.94 (s, 1H), 4.17 (s, 6H), 3.65-3.56 (m, 2H), 2.99 (s, 3H), 2.84-2.69 (m, 1H), 2.48-2.35 (m, 1H).

### Example 137: (R)-2-(3-(5-(3-Hydroxy-1-methyl-2-oxopyrrolidin-3-yl)isoxazol-3-yl)phenyl)-5-((1-methyl-1H-pyrazol-4-yl)amino)pyrimidine-4-carboxamide

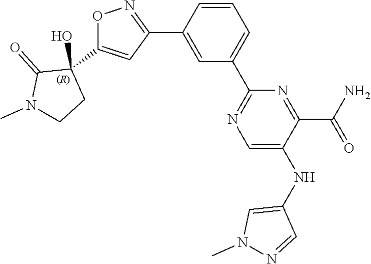

(R)-2-(3-(5-(3-Hydroxy-1-methyl-2-oxopyrrolidin-3-yl)isoxazol-3-yl)phenyl)-5-((1-methyl-1H-pyrazol-4-yl)amino)pyrimidine-4-carboxamide (98 mg) was prepared in a manner analogous to Example 130 using 1-methyl-1H-pyrazol-4-amine instead of 1-methyl-1H-pyrazol-3-ylamine. MS (ESI): Mass calcd. for C₂₃H₂₂N₈O₄, 474.2; m/z found, 475.2 [M+H]⁺. ¹H NMR (600 MHz, CD₃OD) δ 8.85 (t, J=1.6 Hz, 1H), 8.66 (s, 1H), 8.53-8.47 (m, 1H), 7.94-7.88 (m, 1H), 7.79 (d, J=0.8 Hz, 1H), 7.61 (t, J=7.8 Hz, 1H), 7.57 (d, J=0.9 Hz, 1H), 7.03 (s, 1H), 3.95 (s, 3H), 3.66-3.55 (m, 2H), 2.99 (s, 3H), 2.83-2.73 (m, 1H), 2.50-2.38 (m, 1H).

### Example 138: (R)-5-(3-(5-(3-Hydroxy-1-methyl-2-oxopyrrolidin-3-yl)isoxazol-3-yl)phenyl)-2-methoxypyrimidine-4-carboxamide

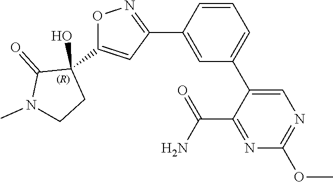

Step A: Ethyl (R)-2-chloro-5-(3-(5-(3-hydroxy-1-methyl-2-oxopyrrolidin-3-yl)isoxazol-3-yl)phenyl)pyrimidine-4-carboxylate. The mixture of ethyl 5-bromo-2-chloropyrimidine-4-carboxylate (265 mg, 0.998 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 383 mg, 0.998 mmol), potassium phosphate (648 mmol, 2.99 mmol) and 1,1′-bis(diphenylphosphino)ferrocene-palladium(II)dichloride dichloromethane complex (46 mg, 0.056 mmol) in THF (5 mL)/H₂O (1 mL) was heated at 100° C. for 2 h. Then it was cooled, and the organic layer was separated and concentrated providing the title compound (300 mg, 67.8%). MS (ESI): Mass calcd. for C₂₁H₁₉N₄O₅, 442.1; m/z found, 443.1 [M+H]⁺.

Step B: (R)-5-(3-(5-(3-Hydroxy-1-methyl-2-oxopyrrolidin-3-yl)isoxazol-3-yl)phenyl)-2-methoxypyrimidine-4-carboxamide. Ethyl (R)-2-chloro-5-(3-(5-(3-hydroxy-1-methyl-2-oxopyrrolidin-3-yl)isoxazol-3-yl)phenyl)pyrimidine-4-carboxylate (200 mg, 0.45 mmol) in NH₃ (7 M in MeOH) (2.25 mL, 15.8 mmol) was heated at 70° C. for 16 h. Then it was cooled to RT during which time a precipitate formed. After filtration, the solids were collected, re-dissolved in DMSO and purified with HPLC reverse phased (CH₃CN/H₂O) providing the title compound (163 mg, 88.1%). MS (ESI): Mass calcd. for C₂₀H₁₉N₅O₅, 409.1; m/z found, 410.1 [M+H]⁺. ¹H NMR (500 MHz, CD₃OD) δ 8.73 (s, 1H), 8.01-7.83 (m, 2H), 7.63-7.52 (m, 2H), 6.92 (s, 1H), 4.13 (s, 3H), 3.67-3.49 (m, 2H), 2.98 (s, 3H), 2.83-2.69 (m, 1H), 2.51-2.38 (m, 1H).

### Example 139: (R)-5-(3-(5-(3-Hydroxy-1-methyl-2-oxopyrrolidin-3-yl)isoxazol-3-yl)phenyl)-1H-pyrrolo[2,3-c]pyridine-7-carboxamide

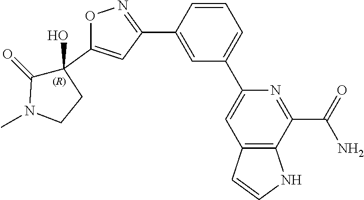

Step A: 5-Chloro-7-(1-ethoxyvinyl)-1H-pyrrolo[2,3-c]pyridine. The mixture of 7-bromo-5-chloro-1H-pyrrolo[2,3-c]pyridine (2.31 g, 9.99 mmol), tributyl(1-ethoxyvinyl)tin (4.87 g, 13.5 mmol) and bis(triphenylphosphine)palladium(ii) chloride (715 mg, 0.998 mmol) in DMF (5 mL) was heated at 70° C. for 3 h. It was cooled down and an aqueous solution of KF (2 mL, 10 M) was added. The mixture was shaken well. Then H₂O (100 mL) was added. Semi-solids were formed. The aqueous layer was decanted. The semi-solids were re-dissolved into THF (100 mL). The in-soluble material was filtered off. The organic layer was concentrated to provide the title compound (2.1 g, 94%). MS (ESI): Mass calcd. for C₁₁H₁₁ClN₂O, 223.1; m/z found, 224.1 [M+H]⁺.

Step B: Ethyl 5-chloro-1H-pyrrolo[2,3-c]pyridine-7-carboxylate. A solution of NaIO₄ (2.47 g, 11.6 mmol) in H₂O (100 mL) was added to a solution of 5-chloro-7-(1-ethoxyvinyl)-1H-pyrrolo[2,3-c]pyridine (647 mg, 2.89 mmol) in dioxane (100 mL). To this mixture was added a solution of KMnO₄ (182 mg, 1.16 mmol) in H₂O (10 mL), and the resulting mixture was stirred for 16 h at 50° C. The solids were filtered out. The resulting solution was extracted with DCM (4×100 mL), and the combined organic layers were dried over sodium sulfate, filtered and concentrated under vacuum to afford the title compound (449 mg, 69.2%). MS (ESI): Mass calcd. for C₁₀H₁₉ClN₂O₂, 224.0; m/z found, 225.1 [M+H]⁺.

Step C: (R)-5-(3-(5-(3-Hydroxy-1-methyl-2-oxopyrrolidin-3-yl)isoxazol-3-yl)phenyl)-1H-pyrrolo[2,3-c]pyridine-7-carboxamide. (R)-5-(3-(5-(3-Hydroxy-1-methyl-2-oxopyrrolidin-3-yl)isoxazol-3-yl)phenyl)-1H-pyrrolo[2,3-c]pyridine-7-carboxamide (41 mg) was prepared in a manner analogous to Example 134 using ethyl 5-chloro-1H-pyrrolo[2,3-c]pyridine-7-carboxylate instead of methyl 2-chloro-5H-pyrrolo[3,2-d]pyrimidine-4-carboxylate. MS (ESI): Mass calcd. for C₂₂H₁₉N₅O₄, 417.1; m/z found, 418.1 [M+H]⁺. ¹H NMR (500 MHz, CD₃OD) δ 8.74-8.60 (m, 1H), 8.37 (s, 1H), 8.31-8.24 (m, 1H), 7.90-7.82 (m, 1H), 7.69-7.60 (m, 2H), 7.05 (s, 1H), 6.73 (d, J=3.1 Hz, 1H), 3.66-3.55 (m, 2H), 3.00 (s, 3H), 2.84-2.74 (m, 1H), 2.49-2.40 (m, 1H).

### Example 140: (R)-2-(5-(5-(4,4-Difluoro-3-hydroxy-1-methyl-2-oxopyrrolidin-3-yl)isoxazol-3-yl)-2-fluorophenyl)-5H-pyrrolo[3,2-d]pyrimidine-4-carboxamide

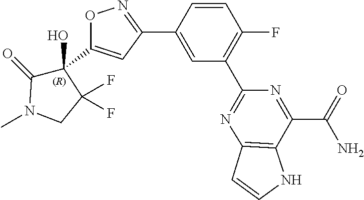

Step A: (R)-(5-(5-(4,4-Difluoro-3-hydroxy-1-methyl-2-oxopyrrolidin-3-yl)isoxazol-3-yl)-2-fluorophenyl)boronic acid. (R)-(5-(5-(4,4-Difluoro-3-hydroxy-1-methyl-2-oxopyrrolidin-3-yl)isoxazol-3-yl)-2-fluorophenyl)boronic acid (500 mg) was prepared in a manner analogous to Intermediate 3 and 4 using 3-bromo-4-fluorobenzaldehyde and (R)-3-ethynyl-4,4-difluoro-3-hydroxy-1-methylpyrrolidin-2-one (Intermediate 44) instead of 3-bromobenzaldehyde and (R)-3-ethynyl-3-hydroxy-1-methylpyrrolidin-2-one. MS (ESI): Mass calcd. for C₁₄H₁₂BF₃N₂O₅, 356.0; m/z found, 356.9 [M+H]⁺.

Step B: (R)-2-(5-(5-(4,4-Difluoro-3-hydroxy-1-methyl-2-oxopyrrolidin-3-yl)isoxazol-3-yl)-2-fluorophenyl)-5H-pyrrolo[3,2-d]pyrimidine-4-carboxamide. (R)-2-(5-(5-(4,4-Difluoro-3-hydroxy-1-methyl-2-oxopyrrolidin-3-yl)isoxazol-3-yl)-2-fluorophenyl)-5H-pyrrolo[3,2-d]pyrimidine-4-carboxamide (13 mg) was prepared in a manner analogous to Example 134 using (R)-(5-(5-(4,4-difluoro-3-hydroxy-1-methyl-2-oxopyrrolidin-3-yl)isoxazol-3-yl)-2-fluorophenyl)boronic acid (Example 119, Step A) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one. MS (ESI): Mass calcd. for C₂₁H₁₅F₃N₆O₄, 472.1; m/z found, 473.1 [M+H]⁺. ¹H NMR (500 MHz, CD₃OD) δ 8.62 (dd, J=7.2, 2.3 Hz, 1H), 8.08-7.93 (m, 2H), 7.48-7.39 (m, 1H), 7.17 (s, 1H), 6.83 (d, J=3.2 Hz, 1H), 4.17-3.90 (m, 2H), 3.07 (s, 3H).

### Example 141: (R)-5-((1H-Pyrazol-4-yl)amino)-2-(3-(5-(3-hydroxy-1-methyl-2-oxopyrrolidin-3-yl)isoxazol-3-yl)phenyl)pyrimidine-4-carboxamide

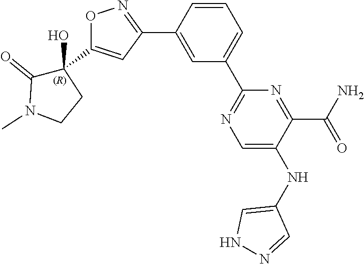

(R)-5-((1H-Pyrazol-4-yl)amino)-2-(3-(5-(3-hydroxy-1-methyl-2-oxopyrrolidin-3-yl)isoxazol-3-yl)phenyl)pyrimidine-4-carboxamide (20 mg) was prepared in a manner analogous to Example 130 using 1H-pyrazol-4-amine instead of 1-methyl-1H-pyrazol-3-ylamine. MS (ESI): Mass calcd. for C₂₂H₂₀N₈O₄, 460.2; m/z found, 461.1 [M+H]⁺. ¹H NMR (500 MHz, CD₃OD) δ 8.91-8.77 (m, 1H), 8.64 (s, 1H), 8.54-8.45 (m, 1H), 7.94-7.87 (m, 1H), 7.76 (s, 1H), 7.64-7.57 (m, 1H), 7.03 (s, 1H), 3.67-3.55 (m, 2H), 2.99 (s, 3H), 2.83-2.73 (m, 1H), 2.51-2.34 (m, 1H).

### Example 142: (R)-5-((3,5-Dimethyl-1H-pyrazol-4-yl)amino)-2-(3-(5-(3-hydroxy-1-methyl-2-oxopyrrolidin-3-yl)isoxazol-3-yl)phenyl)pyrimidine-4-carboxamide

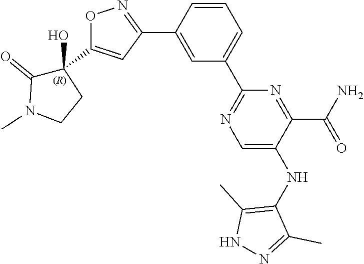

((R)-5-((3,5-Dimethyl-1H-pyrazol-4-yl)amino)-2-(3-(5-(3-hydroxy-1-methyl-2-oxopyrrolidin-3-yl)isoxazol-3-yl)phenyl)pyrimidine-4-carboxamide (1.5 mg) was prepared in a manner analogous to Example 130 using 3,5-dimethyl-1H-pyrazol-4-amine instead of 1-methyl-1H-pyrazol-3-ylamine. MS (ESI): Mass calcd. for C₂₄H₂₄N₈O₄, 488.2; m/z found, 489.6 [M+H]⁺. ¹H NMR (600 MHz, CD₃OD) δ 8.86 (t, J=1.7 Hz, 1H), 8.56-8.44 (m, 1H), 8.20 (s, 1H), 7.93-7.86 (m, 1H), 7.61 (t, J=7.8 Hz, 1H), 7.03 (s, 1H), 3.68-3.51 (m, 2H), 2.99 (s, 3H), 2.83-2.70 (m, 1H), 2.50-2.40 (m, 1H), 2.18 (s, 6H).

### Example 143: (R)-5-(3-(5-(3-hydroxy-1-methyl-2-oxopyrrolidin-3-yl)isoxazol-3-yl)phenyl)-1H-pyrazolo[3,4-c]pyridine-7-carboxamide

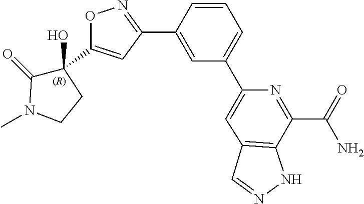

Step A: Ethyl 5-chloro-1H-pyrazolo[3,4-c]pyridine-7-carboxylate. Ethyl 5-chloro-1H-pyrazolo[3,4-c]pyridine-7-carboxylate (510 mg) was prepared in a manner analogous to Example 139 (Step A and B) using 5,7-dichloro-1H-pyrazolo[3,4-c]pyridine instead of 7-bromo-5-chloro-1H-pyrrolo[2,3-c]pyridine MS (ESI): Mass calcd. for C₉HClN₃O₂, 225.1; m/z found, 226.1 [M+H]⁺.

Step B: (R)-3-Hydroxy-1-methyl-3-(3-(3-(trimethylstannyl)phenyl)isoxazol-5-yl)pyrrolidin-2-one. The mixture of (R)-3-(3-(3-bromophenyl)isoxazol-5-yl)-3-hydroxy-1-methylpyrrolidin-2-one (2.02 g, 6.00 mmol), hexamethylditin (1.96 g, 6.00 mmol) and tetrakis(triphenylphosphine)palladium(0) (693 mg, 0.6 mmol) in 1,4-dioxane (12 mL) was heated at 110° C. for 16 h. It was cooled to rt, filtered and purified with HPLC reverse phase (CH₃CN/H₂O) to provide the title compound (1.1 g, 44%). MS (ESI): Mass calcd. for C₁₇H₂₂N₂O₃Sn, 422.0; m/z found, 423.0 [M+H]⁺. ¹H NMR (500 MHz, CD₃OD) δ 7.79-7.69 (m, 1H), 7.59-7.45 (m, 1H), 7.46-7.34 (m, 1H), 7.29-7.18 (m, 1H), 6.68 (s, 1H), 3.45-3.30 (m, 2H), 2.77 (s, 3H), 2.60-2.48 (m, 1H), 2.28-2.12 (m, 1H), 0.13 (s, 9H).

Step C: Ethyl (R)-5-(3-(5-(3-hydroxy-1-methyl-2-oxopyrrolidin-3-yl)isoxazol-3-yl)phenyl)-1H-pyrazolo[3,4-c]pyridine-7-carboxylate. A mixture of (R)-3-hydroxy-1-methyl-3-(3-(3-(trimethylstannyl)phenyl)isoxazol-5-yl)pyrrolidin-2-one (210 mg, 0.50 mmol), ethyl 5-chloro-1H-pyrazolo[3,4-c]pyridine-7-carboxylate (113 mg, 0.499 mmol) and bis(triphenylphosphine)palladium(II) chloride (36 mg, 0.050 mmol) in DMF (0.5 mmol) was heated at 150° C. for 1 h. After the reaction mixture cooled to RT it was filtered and purified with HPLC reverse phase (CH₃CN/H₂O) to provide the title compound (89 mg, 39%). MS (ESI): Mass calcd. for C₂₃H₂₁N₅O₅, 447.2; m/z found, 448.1 [M+H]⁺.

Step D: (R)-5-(3-(5-(3-Hydroxy-1-methyl-2-oxopyrrolidin-3-yl)isoxazol-3-yl)phenyl)-1H-pyrazolo[3,4-c]pyridine-7-carboxamide. A mixture of ethyl (R)-5-(3-(5-(3-hydroxy-1-methyl-2-oxopyrrolidin-3-yl)isoxazol-3-yl)phenyl)-1H-pyrazolo[3,4-c]pyridine-7-carboxylate (89 mg, 0.20 mmol) in NH₃ (7 M in MeOH) (1.0 mL, 7 mmol) was heated at 100° C. for 16 h and then cooled to RT. The mixture was concentrated and purified with reverse phase HPLC (CH₃CN/H₂O) which provided the title compound (7 mg, 8%). MS (ESI): Mass calcd. for C₂₁H₁₈N₆O₄, 418.1; m/z found, 419.4 [M+H]⁺. ¹H NMR (600 MHz, CD₃OD) δ 8.72 (t, J=1.8 Hz, 1H), 8.58 (s, 1H), 8.38-8.27 (m, 2H), 7.95-7.87 (m, 1H), 7.65 (t, J=7.8 Hz, 1H), 7.07 (s, 1H), 3.68-3.55 (m, 2H), 3.00 (s, 3H), 2.83-2.74 (m, 1H), 2.50-2.39 (m, 1H).

### Example 144: (R)-2-(3-(5-(3-Hydroxy-1-methyl-2-oxopyrrolidin-3-yl)isoxazol-3-yl)phenyl)-5-((4-methoxy-1-methyl-1H-pyrazol-3-yl)amino)pyrimidine-4-carboxamide

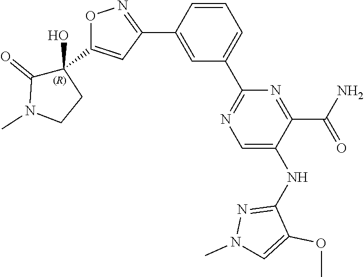

(R)-2-(3-(5-(3-Hydroxy-1-methyl-2-oxopyrrolidin-3-yl)isoxazol-3-yl)phenyl)-5-((4-methoxy-1-methyl-1H-pyrazol-3-yl)amino)pyrimidine-4-carboxamide (11 mg) was prepared in a manner analogous to Example 130 using 4-methoxy-1-methyl-1H-pyrazol-3-amine instead of 1-methyl-1H-pyrazol-3-ylamine. MS (ESI): Mass calcd. for C₂₄H₂₄N₈O₅, 504.2; m/z found, 505.4 [M+H]⁺. ¹H NMR (500 MHz, CD₃OD) δ 9.57 (s, 1H), 8.89 (t, J=1.8 Hz, 1H), 8.60-8.49 (m, 1H), 7.97-7.86 (m, 1H), 7.63 (t, J=7.8 Hz, 1H), 7.40 (s, 1H), 7.05 (s, 1H), 3.84-3.82 (m, 6H), 3.66-3.56 (m, 2H), 3.00 (s, 3H), 2.84-2.74 (m, 1H), 2.50-2.40 (m, 1H).

### Example 145: (R)-2-(3-(5-(3-Hydroxy-1-methyl-2-oxopyrrolidin-3-yl)isoxazol-3-yl)phenyl)-5-((1-(2,2,2-trifluoroethyl)-1H-pyrazol-4-yl)amino)pyrimidine-4-carboxamide

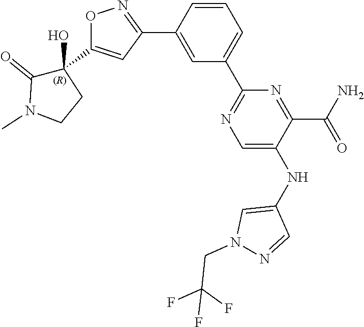

(R)-2-(3-(5-(3-Hydroxy-1-methyl-2-oxopyrrolidin-3-yl)isoxazol-3-yl)phenyl)-5-((1-(2,2,2-trifluoroethyl)-1H-pyrazol-4-yl)amino)pyrimidine-4-carboxamide (5 mg) was prepared in a manner analogous to Example 130 using 1-(2,2,2-trifluoroethyl)-1H-pyrazol-4-amine instead of 1-methyl-1H-pyrazol-3-ylamine. MS (ESI): Mass calcd. for C₂₄H₂₁F₃N₈O₄, 542.2; m/z found, 543.6 [M+H]⁺. ¹H NMR (500 MHz, CD₃OD) δ 8.90-8.80 (m, 1H), 8.67 (s, 1H), 8.54-8.47 (m, 1H), 7.98 (d, J=0.8 Hz, 1H), 7.94-7.87 (m, 1H), 7.74-7.67 (m, 1H), 7.64-7.59 (m, 1H), 7.03 (s, 1H), 4.99 (q, J=8.7 Hz, 2H), 3.65-3.55 (m, 2H), 2.99 (s, 3H), 2.82-2.72 (m, 1H), 2.47-2.40 (m, 1H).

### Example 146: (R)-2-(3-(5-(3-hydroxy-1-methyl-2-oxopyrrolidin-3-yl)isoxazol-3-yl)phenyl)pyrimidine-4-carboxamide

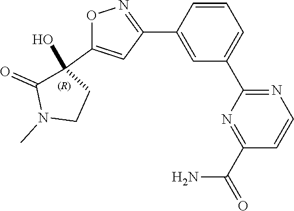

A solution of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 122 mg, 0.32 mmol), 2-chloropyrimidine-4-carboxamide (60 mg, 0.38 mmol), potassium phosphate (202 mg, 0.95 mmol) and bis(tri-tert-butylphosphine)palladium(0) (16 mg, 0.032 mmol) in DMF (2 mL) and water (2 mL) was heated to 90° C. under and N₂ atmosphere for 12 h. The reaction mixture was cooled to rt, filtered and purified by preparative HPLC (Xtimate C18 10 μm, 21.2×250 mm, mobile phase (10 mM NH₄HCO₃ in water+0.025% NH₃H₂O/ACN, Gradient 33% to 48% ACN over 7 min, 15 min run). Detection, UV at 25° C.=214/254 nM) to afford (R)-2-(3-(5-(3-hydroxy-1-methyl-2-oxopyrrolidin-3-yl)isoxazol-3-yl)phenyl)pyrimidine-4-carboxamide (25 mg, 21% yield) as a brown solid. MS (ESI): Mass calcd. for C₁₉H₁₇N₅O₄, 379.37; m/z found, 380.0 [M+H]⁺. ¹H NMR (400 MHz, DMSO-d₆) δ 9.18 (d, J=4.8 Hz, 1H), 9.01 (s, 1H), 8.77 (d, J=8.0 Hz, 1H), 8.76 (s, 1H), 8.07 (t, J=8.0 Hz, 1H), 8.06 (s, 1H), 7.96 (d, J=4.8 Hz, 1H), 7.70 (t, J=8.0 Hz, 1H), 7.21 (s, 1H), 6.75 (s, 1H), 3.40-3.52 (m, 2H), 2.85 (s, 3H), 2.55-2.63 (m, 1H), 2.25-2.33 (m, 1H).

### Example 147: (R)-6-(3-(5-(3-Hydroxy-1-methyl-2-oxopyrrolidin-3-yl)isoxazol-3-yl)phenyl)pyrazine-2-carboxamide

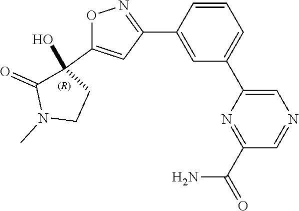

The title compound was prepared in a manner analogous to Example 146 using 6-chloropyrazine-2-carboxamide in place of 2-chloropyrimidine-4-carboxamide to afford (R)-6-(3-(5-(3-hydroxy-1-methyl-2-oxopyrrolidin-3-yl)isoxazol-3-yl)phenyl)pyrazine-2-carboxamide (12 mg, 12% yield) as a colorless solid. MS (ESI): Mass calcd. for C₁₉H₁₇N₅O₄, 379.37; m/z found, 380 [M+H]⁺. ¹H NMR (400 MHz, DMSO-d₆) δ 9.63 (s, 1H), 9.17 (s, 1H), 8.77 (s, 1H), 8.63 (s, 1H), 8.55 (d, J=8.0 Hz, 1H), 8.07 (d, J=8.0 Hz, 1H), 7.98 (s, 1H), 7.71 (t, J=8.0 Hz, 1H), 7.33 (s, 1H), 6.79 (s, 1H), 3.40-3.52 (m, 2H), 2.86 (s, 3H), 2.52-2.62 (m, 1H), 2.23-2.33 (m, 1H).

### Example 148: (R)-6-(3-(5-(3-Hydroxy-1-methyl-2-oxopyrrolidin-3-yl)isoxazol-3-yl)phenyl)picolinamide

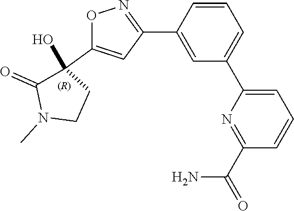

The title compound was prepared in a manner analogous to Example 146 using 6-chloropicolinamide in place of 2-chloropyrimidine-4-carboxamide to afford (R)-6-(3-(5-(3-hydroxy-1-methyl-2-oxopyrrolidin-3-yl)isoxazol-3-yl)phenyl)picolinamide (19 mg, 15% yield) as a colorless solid. MS (ESI): Mass calcd. for C₂₀H₁₈N₄O₄, 378.12; m/z found, 379 [M+H]⁺. ¹H NMR (400 MHz, DMSO-d₆) δ 8.55 (s, 1H), 8.43-8.48 (m, 3H), 8.33 (d, J=8.0 Hz, 1H), 8.10 (t, J=8.0 Hz, 1H), 7.99-8.04 (m, 2H), 7.76 (s, 1H), 7.66 (t, J=8.0 Hz, 1H), 7.28 (s, 1H), 6.75 (s, 1H), 3.43-3.50 (m, 2H), 2.85 (s, 2H), 2.55-2.59 (m, 1H), 2.27-2.32 (m, 1H).

### Example 149: (S)-6-(3-(5-(3-Hydroxy-1-methyl-2-oxopyrrolidin-3-yl)isoxazol-3-yl)phenyl)picolinamide

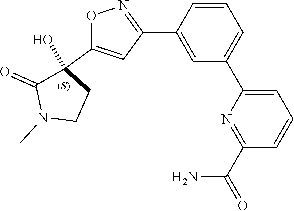

The title compound was prepared in a manner analogous to Example 146 using (S)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 27) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 6-chloropicolinamide in place of 2-chloropyrimidine-4-carboxamide to afford (S)-6-(3-(5-(3-hydroxy-1-methyl-2-oxopyrrolidin-3-yl)isoxazol-3-yl)phenyl)picolinamide (37 mg, 16% yield) as a colorless solid. MS (ESI): Mass calcd. for C₂₀H₁₈N₄O₄, 378.12; m/z found, 379.1 [M+H]⁺. ¹H NMR (400 MHz, DMSO-d₆) δ 8.55 (s, 1H), 8.43-8.48 (m, 2H), 8.33 (dd, J=0.8, 8.0 Hz, 1H), 8.10 (t, J=8.0 Hz, 1H), 7.99-8.04 (m, 2H), 7.76 (s, 1H), 7.66 (t, J=8.0 Hz, 1H), 7.28 (s, 1H), 6.75 (s, 1H), 3.41-3.52 (m, 2H), 2.85 (s, 3H), 2.54-2.61 (m, 1H), 2.25-2.32 (m, 1H).

### Example 150: (R)-6-(3-(5-(3-Hydroxy-1-methyl-2-oxopyrrolidin-3-yl)isoxazol-3-yl)-4-methylphenyl)picolinamide

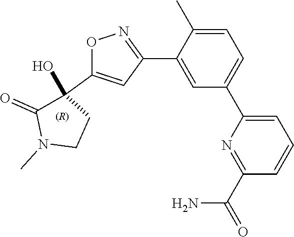

The title compound was prepared in a manner analogous to Example 146 using (R)-3-hydroxy-1-methyl-3-(3-(2-methyl-5-(4,4,5,5-tetramethyl-1,3,2-dioxaborolan-2-yl)phenyl)isoxazol-5-yl)pyrrolidin-2-one (Intermediate 29) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 6-chloropicolinamide in place of 2-chloropyrimidine-4-carboxamide to afford (R)-6-(3-(5-(3-hydroxy-1-methyl-2-oxopyrrolidin-3-yl)isoxazol-3-yl)-4-methylphenyl)picolinamide (25 mg, 15% yield) as a colorless solid. MS (ESI): Mass calcd. for C₂₁H₂₀N₄O₄, 392.41; m/z found, 393.0. [M+H]⁺. ¹H NMR (400 MHz, DMSO-d₆) δ 8.41 (s, 1H), 8.33-8.40 (m, 2H), 8.27 (d, J=8.0 Hz, 1H), 8.06 (t, J=8.0 Hz, 1H), 7.98 (d, J=8.0 Hz, 1H), 7.75 (s, 1H), 7.51 (d, J=8.8 Hz, 1H), 7.03 (s, 1H), 6.75 (s, 1H), 3.38-3.45 (m, 2H), 2.85 (s, 3H), 2.52-2.66 (m, 1H), 2.22-2.35 (m, 1H).

### Example 151: (R)-6-(5-(5-(3-Hydroxy-1-methyl-2-oxopyrrolidin-3-yl)isoxazol-3-yl)-2-methylphenyl)picolinamide

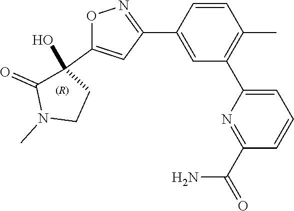

The title compound was prepared in a manner analogous to Example 146 using (R)-3-hydroxy-1-methyl-3-(3-(4-methyl-3-(4,4,5,5-tetramethyl-1,3,2-dioxaborolan-2-yl)phenyl)isoxazol-5-yl)pyrrolidin-2-one (Intermediate 30) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 6-chloropicolinamide in place of 2-chloropyrimidine-4-carboxamide to afford (R)-6-(5-(5-(3-hydroxy-1-methyl-2-oxopyrrolidin-3-yl)isoxazol-3-yl)-2-methylphenyl)picolinamide (31 mg, 19% yield) as a colorless solid. MS (ESI): Mass calcd. for C₂₁H₂₀N₄O₄, 392.41; m/z found, 393.0. [M+H]⁺. ¹H NMR (400 MHz, DMSO-d₆) δ 8.00-8.20 (m, 3H), 7.95 (s, 1H), 7.80-7.94 (m, 2H), 7.70 (s, 1H), 7.49 (d, J=6.0 Hz, 1H), 7.10 (s, 1H), 6.71 (s, 1H), 3.40-3.60 (m, 2H), 2.83 (s, 3H), 2.50-2.60 (m, 1H), 2.42 (s, 3H), 2.20-2.40 (m, 1H).

### Example 152: (R)-6-(3-(5-(3-Hydroxy-1-methyl-2-oxopyrrolidin-3-yl)isoxazol-3-yl)-5-methylphenyl)picolinamide

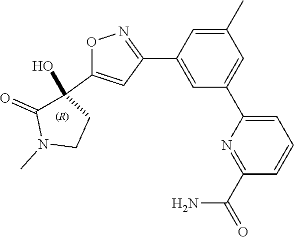

The title compound was prepared in a manner analogous to Example 146 using (R)-3-hydroxy-1-methyl-3-(3-(3-methyl-5-(4,4,5,5-tetramethyl-1,3,2-dioxaborolan-2-yl)phenyl)isoxazol-5-yl)pyrrolidin-2-one (Intermediate 28) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 6-chloropicolinamide in place of 2-chloropyrimidine-4-carboxamide to afford (R)-6-(3-(5-(3-hydroxy-1-methyl-2-oxopyrrolidin-3-yl)isoxazol-3-yl)-5-methylphenyl)picolinamide (73 mg, 25% yield) as a colorless solid. MS (ESI): Mass calcd. for C₂₁H₂₀N₄O₄, 392.41; m/z found, 393.1 [M+H]⁺. ¹H NMR (400 MHz, DMSO-d₆) δ 8.44 (s, 2H), 8.30-8.32 (m, 2H), 8.08 (t, J=7.6 Hz, 1H), 8.02 (d, J=7.6 Hz, 1H), 7.82 (s, 1H), 7.78 (s, 1H), 7.25 (s, 1H), 6.74 (s, 1H), 3.40-3.51 (m, 2H), 2.85 (s, 3H), 2.54-2.60 (m, 1H), 2.50 (s, 3H), 2.25-2.32 (m, 1H).

### Example 153: (R)-6-(3-(5-(3-Hydroxy-1-methyl-2-oxopyrrolidin-3-yl)isoxazol-3-yl)-5-methoxyphenyl)picolinamide

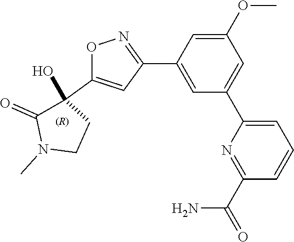

The title compound was prepared in a manner analogous to Example 146 using (R)-3-hydroxy-3-(3-(3-methoxy-5-(4,4,5,5-tetramethyl-1,3,2-dioxaborolan-2-yl)phenyl)isoxazol-5-yl)-1-methylpyrrolidin-2-one (Intermediate 31) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 6-chloropicolinamide in place of 2-chloropyrimidine-4-carboxamide to afford (R)-6-(3-(5-(3-hydroxy-1-methyl-2-oxopyrrolidin-3-yl)isoxazol-3-yl)-5-methoxyphenyl)picolinamide (26 mg, 17% yield) as a colorless solid. MS (ESI): Mass calcd. for C₂₁H₂₀N₄O₅, 408.41; m/z found, 409.1 [M+H]⁺. ¹H NMR (400 MHz, DMSO-d₆) δ 8.44 (s, 1H), 8.32 (d, J=7.2 Hz, 1H), 8.23 (s, 1H), 8.10 (t, J=7.6 Hz, 1H), 8.03 (d, J=7.2 Hz, 1H), 7.99 (s, 1H), 7.75 (s, 1H), 7.52 (s, 1H), 7.29 (s, 1H), 6.74 (s, 1H), 3.95 (s, 3H), 3.41-3.51 (m, 2H), 2.85 (s, 3H), 2.54-2.61 (m, 1H), 2.26-2.32 (m, 1H).

### Example 154: (R)-6-(3-(5-(3-Hydroxy-1-methyl-2-oxopyrrolidin-3-yl)isoxazol-3-yl)phenyl)-3-methylpicolinamide

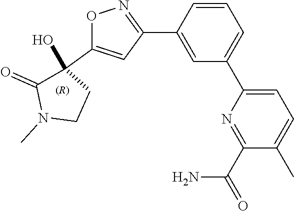

The title compound was prepared in a manner analogous prepared in a manner analogous to Example 146 using 6-chloro-3-methylpicolinamide in place of 2-chloropyrimidine-4-carboxamide to afford (R)-6-(3-(5-(3-hydroxy-1-methyl-2-oxopyrrolidin-3-yl)isoxazol-3-yl)phenyl)-3-methylpicolinamide (25 mg, 14% yield) as a colorless solid. MS (ESI): Mass calcd. for C₂₁H₂₀N₄O₄, 392.41; m/z found, 393.2 [M+H]⁺. ¹H NMR (400 MHz, DMSO-d₆) δ 8.63 (s, 1H), 8.47 (d, J=7.6 Hz, 1H), 8.41 (s, 1H), 8.19 (s, 1H), 7.99 (d, J=7.6 Hz, 1H), 7.88 (s, 1H), 7.73 (s, 1H), 7.65 (t, J=8.0 Hz, 1H), 7.28 (s, 1H), 3.42-3.50 (m, 2H), 2.85 (s, 3H), 2.54-2.60 (m, 1H), 2.52 (s, 3H), 2.27-2.33 (m, 1H).

### Example 155: (R)-6-(3-(5-(3-Hydroxy-1-methyl-2-oxopyrrolidin-3-yl)isoxazol-3-yl)phenyl)-4-methylpicolinamide

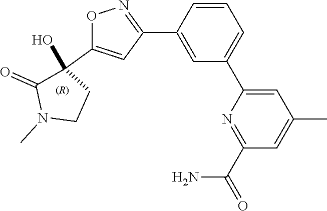

The title compound was prepared in a manner analogous to Example 146 using 6-chloro-4-methylpicolinamide in place of 2-chloropyrimidine-4-carboxamide to afford (R)-6-(3-(5-(3-hydroxy-1-methyl-2-oxopyrrolidin-3-yl)isoxazol-3-yl)phenyl)-4-methylpicolinamide (25 mg, 14% yield) as a colorless solid. MS (ESI): Mass calcd. for C₂₁H₂₀N₄O₄, 392.41; m/z found, 393.2 [M+H]⁺. ¹H NMR (400 MHz, DMSO-d₆) δ 8.63 (s, 1H), 8.47 (d, J=7.6 Hz, 1H), 8.41 (s, 1H), 8.19 (s, 1H), 7.99 (d, J=7.6 Hz, 1H), 7.88 (s, 1H), 7.73 (s, 1H), 7.65 (t, J=8.0 Hz, 1H), 7.28 (s, 1H), 3.42-3.50 (m, 2H), 2.85 (s, 3H), 2.54-2.60 (m, 1H), 2.52 (s, 3H), 2.27-2.33 (m, 1H).

### Example 156: (R)-6-(3-(5-(3-hydroxy-1-methyl-2-oxopyrrolidin-3-yl)isoxazol-3-yl)phenyl)-5-methylpicolinamide

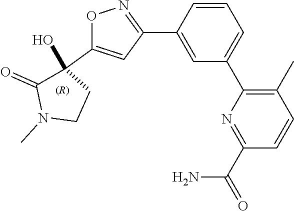

The title compound was prepared in a manner analogous to Example 146 using 6-bromo-5-methylpicolinamide in place of 2-chloropyrimidine-4-carboxamide to afford (R)-6-(3-(5-(3-hydroxy-1-methyl-2-oxopyrrolidin-3-yl)isoxazol-3-yl)phenyl)-5-methylpicolinamide (21 mg, 14% yield) as a colorless solid. MS (ESI): Mass calcd. for C₂₁H₂₀N₄O₄, 392.41; m/z found, 393 [M+H]⁺. ¹H NMR (400 MHz, DMSO-d₆) δ 8.10 (s, 1H), 8.04 (s, 1H), 7.92-8.00 (m, 3H), 7.98 (d, J=7.6 Hz, 1H), 7.60-7.68 (m, 2H), 7.13 (s, 1H), 6.73 (s, 1H), 3.40-3.52 (m, 2H), 2.84 (s, 3H), 2.50-2.62 (m, 1H), 2.43 (s, 3H), 2.23-2.30 (m, 1H).

### Example 157: (R)-3′-(5-(3-Hydroxy-1-methyl-2-oxopyrrolidin-3-yl)isoxazol-3-yl)-[1,1′-biphenyl]-3-carboxamide

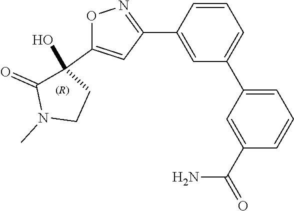

The title compound was prepared in a manner analogous to Example 146 using 3-bromobenzamide in place of 2-chloropyrimidine-4-carboxamide to afford (R)-3′-(5-(3-hydroxy-1-methyl-2-oxopyrrolidin-3-yl)isoxazol-3-yl)-[1,1′-biphenyl]-3-carboxamide (9 mg, 4% yield) as a brown solid. ¹H NMR (400 MHz, DMSO-d₆) δ 8.16-8.25 (m, 3H), 7.80-8.00 (m, 4H), 7.60-7.70 (m, 2H), 7.50 (s, 1H), 7.18 (s, 1H), 6.75 (s, 1H), 3.40-3.52 (m, 2H), 2.85 (s, 3H), 2.50-2.62 (m, 1H), 2.20-2.40 (m, 1H).

### Example 158: (R)-7-Fluoro-2-(3-(5-(3-hydroxy-1-methyl-2-oxopyrrolidin-3-yl)isoxazol-3-yl)phenyl)quinazoline-4-carboxamide

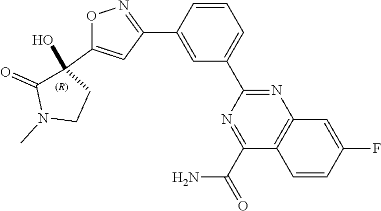

The title compound was prepared in a manner analogous to Example 146 using 2-chloro-7-fluoroquinazoline-4-carboxamide (Intermediate 38) in place of 2-chloropyrimidine-4-carboxamide to afford (R)-7-fluoro-2-(3-(5-(3-hydroxy-1-methyl-2-oxopyrrolidin-3-yl)isoxazol-3-yl)phenyl)quinazoline-4-carboxamide (5 mg, 4% yield) as a colorless solid. MS (ESI): Mass calcd. for C₂₃H₁₈FN₅O₄, 447.42; m/z found, 448 [M+H]⁺. ¹H NMR (400 MHz, DMSO-d₆) δ 9.08 (s, 1H), 9.00 (t, J=2.8 Hz, 1H), 8.70-8.90 (m, 2H), 8.23 (br. s, 1H), 8.11 (d, J=7.6 Hz, 1H), 7.90-8.05 (m, 1H), 7.70-7.80 (m, 2H), 7.18 (s, 1H), 6.78 (s, 1H), 3.40-3.50 (m, 2H), 2.86 (s, 3H), 2.50-2.65 (m, 1H), 2.20-2.40 (m, 1H).

### Example 159: (R)-3-(3-(5-(4-Amino-2-methylpyrido[3,2-d]pyrimidin-6-yl)furan-2-yl)isoxazol-5-yl)-3-hydroxy-1-methylpyrrolidin-2-one

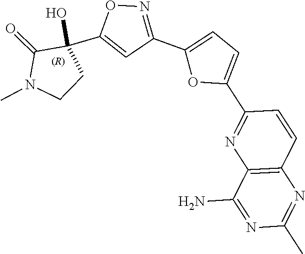

The title compound was prepared in a manner analogous to Example 146 using 6-chloro-2-methylpyrido[3,2-d]pyrimidin-4-amine (Intermediate 16) in place of 2-chloropyrimidine-4-carboxamide and (R)-3-hydroxy-1-methyl-3-(3-(5-(4,4,5,5-tetramethyl-1,3,2-dioxaborolan-2-yl)furan-2-yl)isoxazol-5-yl)pyrrolidin-2-one (Intermediate 32) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one to afford (R)-3-(3-(5-(4-amino-2-methylpyrido[3,2-d]pyrimidin-6-yl)furan-2-yl)isoxazol-5-yl)-3-hydroxy-1-methylpyrrolidin-2-one (55 mg, 22% yield) as a colorless solid. MS (ESI): Mass calcd. for C₂₀H₁₈N₆O₄, 406.40; m/z found, 407.2 [M+H]⁺. ¹H NMR (400 MHz, DMSO-d₆) δ 8.22 (d, J=8.8 Hz, 1H), 8.06 (d, J=8.8 Hz, 1H), 7.97 (br. s, 1H), 7.91 (br. s, 1H), 7.67 (d, J=3.6 Hz, 1H), 7.39 (d, J=3.6 Hz, 1H), 7.05 (s, 1H), 6.79 (s, 1H), 3.39-3.52 (m, 2H), 2.85 (s, 3H), 2.53-2.60 (m, 1H), 2.46 (s, 3H), 2.25-2.32 (m, 1H).

### Example 160: (R)-7-Fluoro-2-(3-(3-(3-hydroxy-1-methyl-2-oxopyrrolidin-3-yl)isoxazol-5-yl)phenyl)quinazoline-4-carboxamide

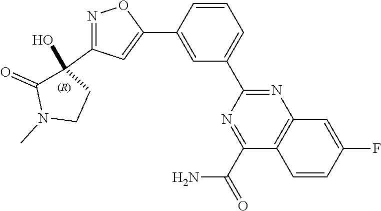

Step A: Ethyl (R)-7-fluoro-2-(3-(3-(3-hydroxy-1-methyl-2-oxopyrrolidin-3-yl)isoxazol-5-yl)phenyl)quinazoline-4-carboxylate. The title compound was prepared in a manner analogous to Example 146 using ethyl 2-chloro-7-fluoroquinazoline-4-carboxylate in place of 2-chloropyrimidine-4-carboxamide and (R)-3-hydroxy-1-methyl-3-(5-(3-(4,4,5,5-tetramethyl-1,3,2-dioxaborolan-2-yl)phenyl)isoxazol-3-yl)pyrrolidin-2-one (Intermediate 5) in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one to afford ethyl (R)-7-fluoro-2-(3-(3-(3-hydroxy-1-methyl-2-oxopyrrolidin-3-yl)isoxazol-5-yl)phenyl)quinazoline-4-carboxamide (80 mg, 36% yield) as a yellow solid. MS (ESI): Mass calcd. for C₂₅H₂₁FN₄O₅, 476.15; m/z found, 477 [M+H]⁺.

Step B: (R)-7-Fluoro-2-(3-(3-(3-hydroxy-1-methyl-2-oxopyrrolidin-3-yl)isoxazol-5-yl)phenyl)quinazoline-4-carboxamide. A solution of ethyl (R)-7-fluoro-2-(3-(3-(3-hydroxy-1-methyl-2-oxopyrrolidin-3-yl)isoxazol-5-yl)phenyl)quinazoline-4-carboxylate (68 mg, 0.14 mmol) in NH₃ in MeOH (2 mL, 7 M, 14 mmol) was heated to 50° C. for 4 h. The reaction mixture was concentrated and purified by prep TLC (EtOAc) followed by reverse phase HPLC using a BOSTON pHlex ODS 21.1×250 mm 10 μm column and mobile phase A: water+0.1% formic acid and B: ACN with a gradient 40-50% B over 8 min at 30 mL/min to afford (R)-7-fluoro-2-(3-(3-(3-hydroxy-1-methyl-2-oxopyrrolidin-3-yl)isoxazol-5-yl)phenyl)quinazoline-4-carboxamide (2.2 mg, 3% yield) as a colorless solid. MS (ESI): Mass calcd. for C₂₃H₁₈FN₅O₄, 447.42; m/z found, 448.2 [M+H]⁺. ¹H NMR (400 MHz, DMSO-d₆) δ 9.00-9.10 (m, 2H), 8.75-8.85 (m, 2H), 8.22 (s, 1H), 8.10 (d, J=7.6 Hz, 1H), 7.99 (dd, J=2.4 and 6.0 Hz, 1H), 7.60-7.80 (m, 2H), 7.27 (s, 1H), 6.57 (s, 1H), 3.30-3.50 (m, 2H), 2.82 (s, 3H), 2.63-2.75 (m, 1H), 2.20-2.32 (m, 1H).

### Example 161: (R,S)-2-(3-(3-(3-Hydroxy-1-methyl-2-oxopyrrolidin-3-yl)isoxazol-5-yl)phenyl)pyrimidine-4-carboxamide

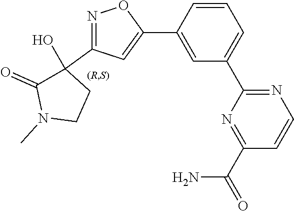

The title compound was prepared in a manner analogous to Example 160 using methyl 2-chloropyrimidine-4-carboxylate in place of ethyl 2-chloro-7-fluoroquinazoline-4-carboxylate and (R,S)-3-hydroxy-1-methyl-3-(5-(3-(4,4,5,5-tetramethyl-1,3,2-dioxaborolan-2-yl)phenyl)isoxazol-3-yl)pyrrolidin-2-one in place of (R)-3-hydroxy-1-methyl-3-(5-(3-(4,4,5,5-tetramethyl-1,3,2-dioxaborolan-2-yl)phenyl)isoxazol-3-yl)pyrrolidin-2-one in Step A to afford (R,S)-2-(3-(3-(3-hydroxy-1-methyl-2-oxopyrrolidin-3-yl)isoxazol-5-yl)phenyl)pyrimidine-4-carboxamide (5 mg, 26%) as a yellow solid. MS (ESI): Mass calcd. for C₁₉H₁₇N₅O₄, 379.37; m/z found, 380 [M+H]+¹H NMR (400 MHz, DMSO-d₆) δ 9.18 (d, J=4.8 Hz, 1H), 9.03 (s, 1H), 8.82 (s, 1H), 8.75 (d, J=8.0 Hz, 1H), 8.08 (s, 1H), 8.06 (s, 1H), 7.98 (d, J=4.8 Hz, 1H), 7.73 (t, J=8.0 Hz, 1H), 7.31 (s, 1H), 6.54 (s, 1H), 3.39-3.48 (m, 2H), 2.82 (s, 3H), 2.65-2.72 (m, 1H), 2.22-2.29 (m, 1H).

### Example 162: (R)-4-(3-(5-(3-Hydroxy-1-methyl-2-oxopyrrolidin-3-yl)isoxazol-3-yl)phenyl)pyrimidine-2-carboxamide

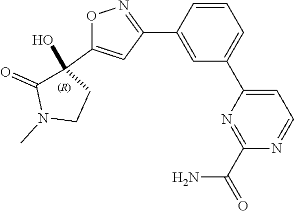

Step A: (R)-3-(3-(3-(2-Chloropyrimidin-4-yl)phenyl)isoxazol-5-yl)-3-hydroxy-1-methylpyrrolidin-2-one. A mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 380 mg, 0.30 mmol), 2,4-dichloropyrimidine (66 mg, 0.45 mmol), potassium phosphate (189 mg, 0.89 mmol) and tetrakis(triphenylphosphine)palladium(0) (17 mg, 0.015 mmol) in DMF (4 mL) and water (1 mL) was heated to 90° C. under and N₂ atmosphere for 16 h. The reaction mixture was cooled to rt, filtered and purified by preparative HPLC (BOSTON PHlex C18 10 m, 21.2×250 mm, mobile phase (47% 0.1% formic acid in water and ACN over 8 min, 14 min run, detection, UV at 25° C.=214/254 nM) to afford (R)-3-(3-(3-(2-chloropyrimidin-4-yl)phenyl)isoxazol-5-yl)-3-hydroxy-1-methylpyrrolidin-2-one (60 mg, 54% yield) as a colorless solid. MS (ESI): Mass calcd. for C₁₈H₁₅ClN₄O₃, 370.08; m/z found, 371 [M+H]⁺.

Step B: Methyl (R)-4-(3-(5-(3-hydroxy-1-methyl-2-oxopyrrolidin-3-yl)isoxazol-3-yl)phenyl)pyrimidine-2-carboxylate. A mixture of (R)-3-(3-(3-(2-chloropyrimidin-4-yl)phenyl)isoxazol-5-yl)-3-hydroxy-1-methylpyrrolidin-2-one (15 mg, 0.041 mmol), [1,1′-bis(diphenylphosphino)ferrocene]dichloropalladium(II), complex with dichloromethane (1.7 mg, 0.002 mmol) was heated to 60° C. under an atmosphere of CO(g) for 16 h. The mixture was filtered and concentrated to dryness to afford methyl (R)-4-(3-(5-(3-hydroxy-1-methyl-2-oxopyrrolidin-3-yl)isoxazol-3-yl)phenyl)pyrimidine-2-carboxylate (15 mg, 94% yield) as a yellow solid. MS (ESI): Mass calcd. for C₂₀H₁₈N₄O₅, 394.12; m/z found, 395 [M+H]⁺.

Step C: (R)-4-(3-(5-(3-Hydroxy-1-methyl-2-oxopyrrolidin-3-yl)isoxazol-3-yl)phenyl)pyrimidine-2-carboxamide. The title compound was prepared in a manner analogous to Example 166 Step B using methyl (R)-4-(3-(5-(3-hydroxy-1-methyl-2-oxopyrrolidin-3-yl)isoxazol-3-yl)phenyl)pyrimidine-2-carboxylate in place of (R)-ethyl 1-(3-(5-(3-hydroxy-1-methyl-2-oxopyrrolidin-3-yl)isoxazol-3-yl)phenyl)-1H-indazole-3-carboxylate to afford (R)-4-(3-(5-(3-hydroxy-1-methyl-2-oxopyrrolidin-3-yl)isoxazol-3-yl)phenyl)pyrimidine-2-carboxamide (2.5 mg, 17% yield) as a colorless solid. MS (ESI): Mass calcd. for C₁₉H₁₇N₅O₄, 379.13; m/z found, 380 [M+H]⁺. ¹H NMR (400 MHz, DMSO-d₆) δ 9.05 (d, J=5.2 Hz, 1H), 8.76 (s, 1H), 8.53 (d, J=9.2 Hz, 1H), 8.46 (s, 1H), 8.39 (d, J=5.2 Hz, 1H), 8.11 (d, J=8.0 Hz, 1H), 7.91 (s, 1H), 7.74 (t, J=8.0 Hz, 1H), 7.26 (s, 1H), 6.78 (s, 1H), 3.40-3.52 (m, 2H), 2.86 (s, 3H), 2.52-2.62 (m, 1H), 2.24-2.35 (m, 1H).

### Example 163: (R)-3-(3-(3-(5H-Pyrrolo[2,3-b]pyrazin-7-yl)phenyl)isoxazol-5-yl)-3-hydroxy-1-methylpyrrolidin-2-one

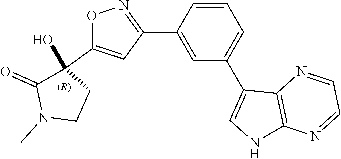

Step A: (R)-3-Hydroxy-1-methyl-3-(3-(3-(5-tosyl-5H-pyrrolo[2,3-b]pyrazin-7-yl)phenyl)isoxazol-5-yl)pyrrolidin-2-one. The title compound was prepared in a manner analogous to Example 146 using 7-(4,4,5,5-tetramethyl-1,3,2-dioxaborolan-2-yl)-5-tosyl-5H-pyrrolo[2,3-b]pyrazine in place of 2-chloropyrimidine-4-carboxamide and (R)-3-(3-(3-bromophenyl)isoxazol-5-yl)-3-hydroxy-1-methylpyrrolidin-2-one in place of (R)-3-hydroxy-1-methyl-3-(3-(3-(4,4,5,5-tetramethyl-1,3,2-dioxaborolan-2-yl)phenyl)isoxazol-5-yl)pyrrolidin-2-one to afford (R)-3-hydroxy-1-methyl-3-(3-(3-(5-tosyl-5H-pyrrolo[2,3-b]pyrazin-7-yl)phenyl)isoxazol-5-yl)pyrrolidin-2-one (160 mg, 51% yield) as a colorless solid. MS (ESI): Mass calcd. for C₂₇H₂₃N₅O₅S, 529.14; m/z found, 530.2 [M+H]⁺.

Step B: ((R)-3-(3-(3-(5H-Pyrrolo[2,3-b]pyrazin-7-yl)phenyl)isoxazol-5-yl)-3-hydroxy-1-methylpyrrolidin-2-one. A solution of (R)-3-hydroxy-1-methyl-3-(3-(3-(5-tosyl-5H-pyrrolo[2,3-b]pyrazin-7-yl)phenyl)isoxazol-5-yl)pyrrolidin-2-one (180 mg, 0.34 mmol), and aq. NaOH (2 mL, 1 M, 2 mmol) in THF (8 mL) and MeOH (2 mL) was stirred at 15° C. for 5 h and then diluted with EtOAc (30 mL) and washed with brine (2×15 mL). The organic layer was separated, dried over Na₂SO₄, filtered and concentrated. The residue was purified by reverse phase HPLC on a Xtimate C18 10 μm, 21.2×250 mm column using 10 mM NH₄HCO₃ water+0.025% NH₃ and ACN; 5% B for 3 min then 30-35% B over 10 min to afford ((R)-3-(3-(3-(5H-pyrrolo[2,3-b]pyrazin-7-yl)phenyl)isoxazol-5-yl)-3-hydroxy-1-methylpyrrolidin-2-one (37 mg, 29% yield) as a pale yellow solid. MS (ESI): Mass calcd. for C₂₀H₁₇N₅O₃, 375.39; m/z found, 376.2 [M+H]⁺. ¹H NMR (400 MHz, DMSO-d₆) δ 2.30 (s, 1H), 8.70 (s, 1H), 8.61 (s, 1H), 8.55 (d, J=2.8 Hz, 1H), 8.42 (d, J=8.0 Hz, 1H), 8.34 (d, J=2.8 Hz, 1H), 7.73 (d, J=8.0 Hz, 1H), 7.57 (t, J=8.0 Hz, 1H), 7.07 (s, 1H), 6.75 (s, 1H), 3.41-3.52 (m, 2H), 2.85 (s, 3H), 2.55-2.62 (m, 1H), 2.25-2.32 (m, 1H).

### Example 164: (R)-6-(5-(5-(3-Hydroxy-1-methyl-2-oxopyrrolidin-3-yl)isoxazol-3-yl)furan-2-yl)picolinamide

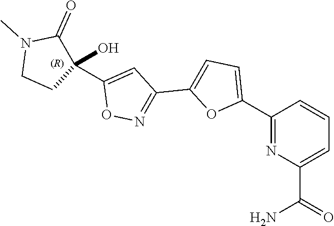

Step A: (R)-Methyl 6-(5-(5-(3-hydroxy-1-methyl-2-oxopyrrolidin-3-yl)isoxazol-3-yl)furan-2-yl)picolinate. A mixture of (R)-3-(3-(5-(6-chloropyridin-2-yl)furan-2-yl)isoxazol-5-yl)-3-hydroxy-1-methylpyrrolidin-2-one (160 mg, 0.45 mmol, Intermediate 33), 1,1′-bis(diphenylphosphino) ferrocene-palladium(II)dichloride dichloromethane complex (18 mg, 0.022 mmol) and triethylamine (90 mg, 0.89 mmol) in MeOH (30 mL) was heated at 60° C. under CO gas for 48 h. The reaction mixture was filtered and the filtrate was concentrated to dryness. The residue was purified by prep-TLC (silica, UV-254, EtOAc) to afford (R)-methyl 6-(5-(5-(3-hydroxy-1-methyl-2-oxopyrrolidin-3-yl)isoxazol-3-yl)furan-2-yl)picolinate (110 mg, 64%) as a yellow oil. MS (ESI): Mass calcd. for C₁₉H₁₇N₃O₆, 383.355; m/z found, 384.1 [M+H]⁺.

Step B: (R)-6-(5-(5-(3-Hydroxy-1-methyl-2-oxopyrrolidin-3-yl)isoxazol-3-yl)furan-2-yl)picolinamide. A mixture of (R)-methyl 6-(5-(5-(3-hydroxy-1-methyl-2-oxopyrrolidin-3-yl)isoxazol-3-yl)furan-2-yl)picolinate (110 mg, 0.29 mmol) and 7 N NH₃ in ethanol (8 mL) was heated at 75° C. for 16 h in a sealed tube. The reaction mixture was concentrated under reduced pressure and the residue was purified by reverse phase HPLC using Xtimate C18 10 μm, 21.2×250 mm column using a 20% to 45% gradient of ACN/10 mM NH₄HCO₃ and 0.025% NH₃H₂O in H₂O; 15 min gradient to afford (R)-6-(5-(5-(3-hydroxy-1-methyl-2-oxopyrrolidin-3-yl)isoxazol-3-yl)furan-2-yl)picolinamide (44 mg, 41%) as a white solid. MS (ESI): Mass calcd. for C₁₈H₁₆N₄O₅, 368.343; m/z found, 369.1 [M+H]⁺. ¹H NMR (400 MHz, DMSO-d₆) δ 8.36 (s, 1H), 8.10 (t, J=7.6 Hz, 1H), 8.02 (d, J=7.8 Hz, 1H), 7.97 (d, J=7.8 Hz, 1H), 7.78 (s, 1H), 7.72 (d, J=3.6 Hz, 1H), 7.38 (d, J=3.6 Hz, 1H), 7.02 (s, 1H), 6.78 (s, 1H), 3.39-3.52 (m, 2H), 2.85 (s, 3H), 2.53-2.60 (m, 1H), 2.25-2.32 (m, 1H).

### Example 165: (R,S)-6-(5-(5-(3-Hydroxy-1-methyl-2-oxopyrrolidin-3-yl)isoxazol-3-yl)furan-2-yl)picolinamide

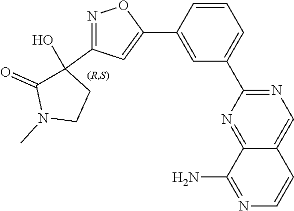

Step A: 3-(5-(3-(8-Chloropyrido[3,4-d]pyrimidin-2-yl)phenyl)isoxazol-3-yl)-3-hydroxy-1-methylpyrrolidin-2-one. To a suspension of 8-chloro-2-(methylthio)pyrido[3,4-d]pyrimidine (60 mg, 0.28 mmol), (3-(3-(3-hydroxy-1-methyl-2-oxopyrrolidin-3-yl)isoxazol-5-yl)phenyl)boronic acid (103 mg, 0.34 mmol, Intermediate 36), and copperthiophenecarboxylate (108 mg, 0.57 mmol) in THF (8 mL) was added bis(dibenzylideneacetone)palladium(0) (26 mg, 0.028 mmol), followed by tri(2-furyl)phosphine (7 mg, 0.028 mmol) under argon. The reaction mixture was heated in a microwave at 80° C. for 4 h. The reaction mixture was diluted with EtOAc (20 mL) and filtered. The filtrate was concentrated and purified by prep-TLC (DCM:MeOH, 20:1) to afford 3-(5-(3-(8-chloropyrido[3,4-d]pyrimidin-2-yl)phenyl)isoxazol-3-yl)-3-hydroxy-1-methylpyrrolidin-2-one (70 mg, 59% yield) as a yellow solid. MS (ESI): Mass calcd. for C₂₁H₁₆ClN₅O₃, 421.09; m/z found, 422.1 [M+H]⁺.

Step B: (R,S)-6-(5-(5-(3-Hydroxy-1-methyl-2-oxopyrrolidin-3-yl)isoxazol-3-yl)furan-2-yl)picolinamide. A solution of 3-(5-(3-(8-chloropyrido[3,4-d]pyrimidin-2-yl)phenyl)isoxazol-3-yl)-3-hydroxy-1-methylpyrrolidin-2-one (60 mg, 0.14 mmol) in dioxane (2 mL) was treated with NH₃ in water (6 mL, 12 M, 72 mmol) and then heated at 70° C. for 16 h. The reaction mixture was cooled to rt, concentrated to dryness and purified by reverse phase HPLC BOSTON pHlex ODS 21.1×250 mm 10 μm using mobile phase A: water+0.1% Formic acid and B: ACN with a gradient 40-50% B over 8 min at 30 mL/min to afford (R,S)-6-(5-(5-(3-hydroxy-1-methyl-2-oxopyrrolidin-3-yl)isoxazol-3-yl)furan-2-yl)picolinamide (5.6 mg, 10% yield). MS (ESI): Mass calcd. for C₂₁H₁₈N₆O₃, 402.41; m/z found, 403.1 [M+H]⁺. ¹H NMR (400 MHz, DMSO-d₆) δ 9.57 (s, 1H), 9.09 (s, 1H), 8.81 (d, J=8.0 Hz, 1H), 8.04 (d, J=8.0 Hz, 1H), 8.03 (d, J=5.6 Hz, 1H), 7.73 (t, J=8.0 Hz, 1H), 7.56 (s, 2H), 7.33 (s, 1H), 7.05 (d, J=5.6 Hz, 1H), 6.54 (s, 1H), 3.40-3.49 (m, 2H), 2.81 (s, 3H), 2.65-2.72 (m, 1H), 2.22-2.29 (m, 1H).

### Example 166: (R)-1-(3-(5-(3-hydroxy-1-methyl-2-oxopyrrolidin-3-yl)isoxazol-3-yl)phenyl)-1H-indazole-3-carboxamide

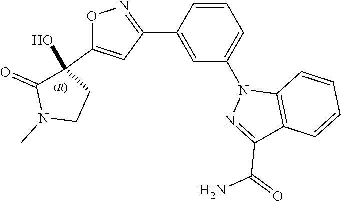

Step A: (R)-Ethyl 1-(3-(5-(3-hydroxy-1-methyl-2-oxopyrrolidin-3-yl)isoxazol-3-yl)phenyl)-1H-indazole-3-carboxylate. A mixture of ethyl 1H-indazole-3-carboxylate (103 mg, 0.55 mmol), (R)-3-(5-(3-hydroxy-1-methyl-2-oxopyrrolidin-3-yl)isoxazol-3-yl)phenylboronic acid (110 mg, 0.36 mmol), Cu(OAc)₂ (66.139 mg, 0.364 mmol) and molecular sieves (500 mg) in chloroform (8 mL) and pyridine (144 mg, 1.82 mmol) was heated at 40° C. for 72 h under 02. The mixture was filtered and concentrated under reduced pressure. The residue was purified by prep-TLC (DCM/EtOAc=3/1) to afford (R)-ethyl 1-(3-(5-(3-hydroxy-1-methyl-2-oxopyrrolidin-3-yl)isoxazol-3-yl)phenyl)-1H-indazole-3-carboxylate (55 mg, 34%) as a white solid. MS (ESI): Mass calcd. for C₂₄H₂₂N₄O₅, 446.45; m/z found, 447.1 [M+H]⁺.

Step B: (R)-1-(3-(5-(3-Hydroxy-1-methyl-2-oxopyrrolidin-3-yl)isoxazol-3-yl)phenyl)-1H-indazole-3-carboxamide. To a sealed tube containing (R)-ethyl 1-(3-(5-(3-hydroxy-1-methyl-2-oxopyrrolidin-3-yl)isoxazol-3-yl)phenyl)-1H-indazole-3-carboxylate (55 mg, 0.12 mmol) was added ammonia (7 N in methanol) (8 mL). The mixture was heated at 75° C. for 16 h and concentrated to dryness. The residue was purified by reverse phase HPLC on a Xtimate C18 10 μm, 21.2×250 mm column using a 20% to 45% gradient of ACN/10 mM NH₄HCO₃ and 0.025% NH₃H₂O in H₂O; 15 min gradient to afford (R)-1-(3-(5-(3-hydroxy-1-methyl-2-oxopyrrolidin-3-yl)isoxazol-3-yl)phenyl)-1H-indazole-3-carboxamide (15.2 mg, 30%) as a white solid. MS (ESI): Mass calcd. for C₂₂H₁₉N₅O₄, 417.42; m/z found, 418.1 [M+H]⁺. ¹H NMR (400 MHz, DMSO-d₆) δ 8.31-8.33 (m, 2H), 7.99-8.05 (m, 3H), 7.93 (d, J=8.0 Hz, 1H), 7.78 (t, J=8.0 Hz, 1H), 7.56-7.60 (m, 2H), 7.41 (t, J=8.0 Hz, 1H), 7.18 (s, 1H), 6.76 (s, 1H), 3.43-3.49 (m, 2H), 2.84 (s, 3H), 2.54-2.61 (m, 1H), 2.25-2.32 (m, 1H).

### Example 167: (R,S)-1-(3-(3-(3-Hydroxy-1-methyl-2-oxopyrrolidin-3-yl)isoxazol-5-yl)phenyl)-1H-indazole-3-carboxamide

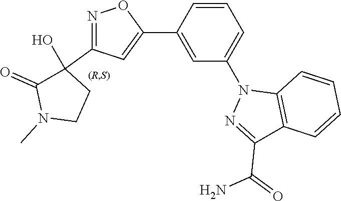

The title compound was prepared in a manner analogous to Example 166 using (R,S)-(3-(3-(3-hydroxy-1-methyl-2-oxopyrrolidin-3-yl)isoxazol-5-yl)phenyl)boronic acid (Intermediate 36) in place of (R)-3-(5-(3-hydroxy-1-methyl-2-oxopyrrolidin-3-yl)isoxazol-3-yl)phenylboronic acid in Step A to afford (R,S)-1-(3-(3-(3-hydroxy-1-methyl-2-oxopyrrolidin-3-yl)isoxazol-5-yl)phenyl)-1H-indazole-3-carboxamide. MS (ESI): Mass calcd. for C₂₂H₁₉N₅O₄, 417.42; m/z found, 418.2 [M+H]⁺. ¹H NMR (400 MHz, DMSO-d₆) δ 8.31-8.35 (m, 2H), 7.95-8.08 (m, 4H), 7.80 (t, J=7.6 Hz, 1H), 7.57-7.61 (m, 2H), 7.42 (t, J=7.6 Hz, 1H), 7.28 (s, 1H), 6.53 (s, 1H), 3.37-3.49 (m, 2H), 2.81 (s, 3H), 2.64-2.70 (m, 1H), 2.22-2.29 (m, 1H).

### Example 168: (R)-4′-(5-(3-Hydroxy-1-methyl-2-oxopyrrolidin-3-yl)isoxazol-3-yl)-[2,2′-bipyridine]-6-carboxamide

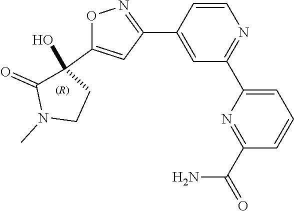

A solution of 6-(tributylstannyl)picolinamide (100 mg, 0.24 mmol, Intermediate 40), (R)-3-(3-(2-bromopyridin-4-yl)isoxazol-5-yl)-3-hydroxy-1-methylpyrrolidin-2-one (Intermediate 7, 82 mg, 0.24 mmol) and tetrakis(triphenylphosphine)palladium(0) (14 mg, 0.012 mmol) in DMF (2 mL) was heated to 125° C. for 16 h under N₂. The mixture was filtered, concentrated, and the residue was purified by reverse phase HPLC on a Xtimate C18 10 μm, 21.2×250 mm column using 10 mM NH₄HCO₃ water+0.025% NH₃ and ACN; 5% B for 3 min then 30-35% B over 10 min to afford (R)-4′-(5-(3-hydroxy-1-methyl-2-oxopyrrolidin-3-yl)isoxazol-3-yl)-[2,2′-bipyridine]-6-carboxamide (16 mg, 18% yield) as a colorless solid. ¹H NMR (400 MHz, DMSO-d₆) δ 9.08 (s, 1H), 8.50 (d, J=4.8 Hz, 1H), 8.71 (s, 1H), 8.20 (d, J=6.0 Hz, 1H), 8.10-8.20 (m, 2H), 7.98 (d, J=8.8 Hz, 1H), 7.83 (s, 1H), 7.49 (s, 1H), 6.84 (s, 1H), 3.40-3.50 (m, 2H), 2.86 (s, 3H), 2.60-2.65 (m, 1H), 2.20-2.40 (m, 1H).

### Example 169: (R)-5-Amino-4′-(5-(3-hydroxy-1-methyl-2-oxopyrrolidin-3-yl)isoxazol-3-yl)-[2,2′-bipyridine]-6-carboxamide

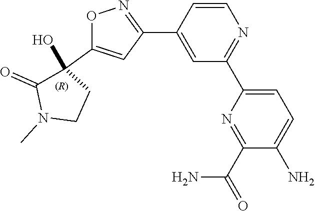

The title compound was prepared analogous to Example 168 using 3-amino-6-(tributylstannyl)picolinamide (Intermediate 41) in place of 6-(tributylstannyl)picolinamide to afford (R)-5-amino-4′-(5-(3-hydroxy-1-methyl-2-oxopyrrolidin-3-yl)isoxazol-3-yl)-[2,2′-bipyridine]-6-carboxamide (6 mg, 7% yield) as a colorless solid. MS (ESI): Mass calcd. for C₁₉H₁₈N₆O₄, 394.39; m/z found, 395 [M+H]⁺. ¹H NMR (400 MHz, DMSO-d₆) δ 8.80 (s, 1H), 8.71 (d, J=4.8 Hz, 1H), 8.46 (s, 1H), 8.31 (d, J=8.4 Hz, 1H), 7.78 (dd, J=1.6, 4.8 Hz, 1H), 7.50 (s, 1H), 7.47 (s, 1H), 7.16-7.35 (m, 3H), 6.80 (s, 1H), 3.40-3.53 (m, 2H), 2.85 (s, 3H), 2.50-2.60 (m, 1H), 2.25-2.35 (m, 1H).

### Example 170: (R)-3-(3-(2-(4-Amino-2-methylpyrido[3,2-d]pyrimidin-6-yl)pyridin-4-yl)isoxazol-5-yl)-3-hydroxy-1-methylpyrrolidin-2-one

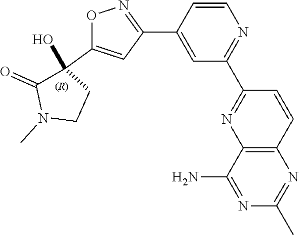

The title compound was prepared analogous to Example 168 using 6-chloro-2-methylpyrido[3,2-d]pyrimidin-4-amine (Intermediate 16) in place of 6-(tributylstannyl)picolinamide and (R)-3-hydroxy-1-methyl-3-(3-(2-(tributylstannyl)pyridin-4-yl)isoxazol-5-yl)pyrrolidin-2-one in place of (R)-3-(3-(2-bromopyridin-4-yl)isoxazol-5-yl)-3-hydroxy-1-methylpyrrolidin-2-one to afford (R)-3-(3-(2-(4-amino-2-methylpyrido[3,2-d]pyrimidin-6-yl)pyridin-4-yl)isoxazol-5-yl)-3-hydroxy-1-methylpyrrolidin-2-one (5 mg, 2% yield) as a colorless solid. MS (ESI): Mass calcd. for C₂₁H₁₉N₇O₃, 417.43; m/z found, 418 [M+H]⁺. ¹H NMR (400 MHz, DMSO-d₆) δ 9.17 (s, 1H), 8.85 (d, J=5.2 Hz, 1H), 8.79 (d, J=9.2 Hz, 1H), 8.43 (s, 1H), 8.11 (d, J=9.2 Hz, 1H), 8.05 (s, 1H), 7.96 (d, J=5.2 Hz, 1H), 7.48 (s, 1H), 6.84 (s, 1H), 3.44-3.53 (m, 2H), 2.86 (s, 3H), 2.56-2.61 (m, 1H), 2.48 (s, 3H), 2.30-2.35 (m, 1H).

### Example 171: (5R,8S)-2-(3-(5-((R)-3-Hydroxy-1-methyl-2-oxopyrrolidin-3-yl)isoxazol-3-yl)phenyl)-6,7,8,9-tetrahydro-5H-5,8-epoxycyclohepta[d]pyrimidine-4-carboxamide

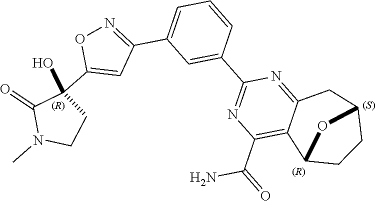

Step A: (5R,8S)-2-(3-((E)-(Hydroxyimino)methyl)phenyl)-6,7,8,9-tetrahydro-5H-5,8-epoxycyclohepta[d]pyrimidine-4-carboxamide. To a solution of (5R,8S)-2-(3-formylphenyl)-6,7,8,9-tetrahydro-5H-5,8-epoxycyclohepta[d]pyrimidine-4-carboxamide (200 mg, 0.647 mmol, Intermediate 37) in EtOH (8 mL) was added NH₂OH (50% in H₂O) (25.6 mg, 0.776 mmol). The mixture was stirred at 30° C. for 4 h. The mixture was concentrated to afford the title compound (202 mg, 96% yield). MS (ESI): Mass calcd. for C₁₇H₁₆N₄O₃, 324.3; m/z found, 324.8 [M+H]⁺.

Step B: (5R,8S)-2-(3-(5-((R)-3-Hydroxy-1-methyl-2-oxopyrrolidin-3-yl)isoxazol-3-yl)phenyl)-6,7,8,9-tetrahydro-5H-5,8-epoxycyclohepta[d]pyrimidine-4-carboxamide. N-Chlorosuccinimide (82.3 mg, 0.617 mmol) was added portion wise to a solution of (5R,8S)-2-(3-((E)-(hydroxyimino)methyl)phenyl)-6,7,8,9-tetrahydro-5H-5,8-epoxycyclohepta[d]pyrimidine-4-carboxamide (100 mg, 0.308 mmol) in dry dichloromethane (4 mL) and the reaction mixture was heated at 30° C. for 16 h. Then (R)-3-ethynyl-3-hydroxy-1-methylpyrrolidin-2-one (Intermediate 1, 42.9 mg, 0.308 mmol) and TEA (93.422 mg, 0.925 mmol) was added. The mixture was heated at 40° C. for 3 h. The mixture was concentrated and purified by reverse phase HPLC on a Xtimate 10 m 150 A, 21.2×250 mm column using a 5 to 40% gradient of MeCN/10 mM NH₄HCO₃ and 0.025% NH₃H₂O in H₂O; 18 min gradient to afford the title compound as a white solid (11.5 mg, 8.1%). MS (ESI): Mass calcd. for C₂₄H₂₃N₅O₅, 461.47; m/z found, 461.8 [M+H]⁺. ¹H NMR (400 MHz, DMSO-d₆) δ 8.90 (s, 1H), 8.66 (d, J=8.0 Hz, 1H), 8.62 (s, 1H), 8.04 (d, J=8.0 Hz, 1H), 7.96 (s, 1H), 7.68 (t, J=8.0 Hz, 1H), 7.16 (s, 1H), 6.76 (s, 1H), 5.90 (d, J=6.0 Hz, 1H), 4.80 (t, J=6.0 Hz, 1H), 3.33-3.52 (m, 2H), 2.80-2.90 (m, 1H), 2.85 (s, 3H), 2.55-2.70 (m, 1H), 2.07-2.32 (m, 4H), 1.78 (br. s, 1H), 1.24 (br. s, 1H).

### Example 172: (5R,8S)-2-(3-(5-((S)-3-Hydroxy-1-methyl-2-oxopyrrolidin-3-yl)isoxazol-3-yl)phenyl)-6,7,8,9-tetrahydro-5H-5,8-epoxycyclohepta[d]pyrimidine-4-carboxamide

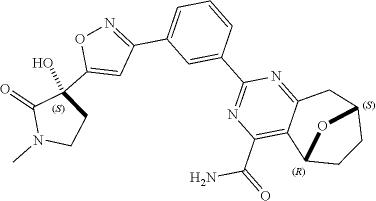

The title compound was prepared in a manner analogous to Example 171 Step B using (S)-3-ethynyl-3-hydroxy-1-methylpyrrolidin-2-one in place of (R)-3-ethynyl-3-hydroxy-1-methylpyrrolidin-2-one to afford (5R,8S)-2-(3-(5-((S)-3-hydroxy-1-methyl-2-oxopyrrolidin-3-yl)isoxazol-3-yl)phenyl)-6,7,8,9-tetrahydro-5H-5,8-epoxycyclohepta[d]pyrimidine-4-carboxamide (20 mg, 6% yield) as a colorless solid. MS (ESI): Mass calcd. for C₂₄H₂₃N₅O₅, 461.47; m/z found, 462.1 [M+H]⁺. ¹H NMR (400 MHz, DMSO-d₆) δ 8.90 (s, 1H), 8.67 (d, J=8.0 Hz, 1H), 8.63 (s, 1H), 8.05 (d, J=6.4 Hz, 1H), 7.97 (s, 1H), 7.67 (t, J=8.0 Hz, 1H), 7.16 (d, J=2.0 Hz, 1H), 6.75 (d, J=2.0 Hz, 1H), 5.89 (d, J=2.4 Hz, 1H), 4.81 (t, J=6.4 Hz, 1H), 3.38-3.51 (m, 3H), 2.82-2.86 (m, 4H), 2.55-2.62 (m, 1H), 2.26-2.32 (m, 1H), 2.16-2.25 (m, 2H), 2.05-2.10 (m, 1H), 1.76-1.78 (m, 1H).

### Example 173: (5R,8S)-2-(3-(5-((R)-3-Hydroxy-1-methyl-2-oxopyrrolidin-3-yl)-1H-pyrazol-3-yl)phenyl)-6,7,8,9-tetrahydro-5H-5,8-epoxycyclohepta[d]pyrimidine-4-carboxamide

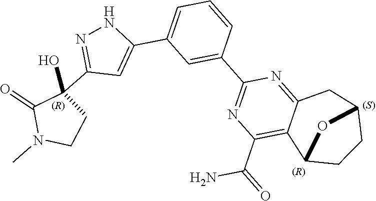

To a solution of p-toluenesulfonyl hydrazide (18 mg, 0.097 mmol) in ACN (3 mL) was added (5R,8S)-2-(3-formylphenyl)-6,7,8,9-tetrahydro-5H-5,8-epoxycyclohepta[d]pyrimidine-4-carboxamide (30 mg, 0.097 mmol, Example 174 Step A) and the reaction mixture was stirred at rt for 3 h. An aqueous solution of NaOH (5 N, 0.02 mL) was then added. The mixture was stirred for 20 min and (R)-3-ethynyl-3-hydroxy-1-methylpyrrolidin-2-one (Intermediate 1, 40.5 mg, 0.291 mmol) was added and heating was continued at 50° C. for 16 h. The crude reaction mixture was concentrated and purified by reverse phase HPLC on an Xtimate 10 m 150 A, 21.2×250 mm column using 30% t of ACN/10 mM NH₄HCO₃ and 0.025% NH₃H₂O in H₂O; 10 min gradient to afford (5R,8S)-2-(3-(5-((R)-3-hydroxy-1-methyl-2-oxopyrrolidin-3-yl)-1H-pyrazol-3-yl)phenyl)-6,7,8,9-tetrahydro-5H-5,8-epoxycyclohepta[d]pyrimidine-4-carboxamide (2 mg, 4.4%) as a yellow solid. MS (ESI): Mass calcd. For C₂₄H₂₄N₆O₄, 460.485; m/z found, 460.7 [M+H]⁺. ¹H NMR (400 MHz, CDCl₃) δ 8.69 (s, 1H), 8.18 (s, 2H), 7.52 (s, 1H), 7.34-7.48 (m, 2H), 6.61 (s, 1H), 6.29 (s, 1H), 6.15 (s, 1H), 4.84 (s, 1H), 3.38-3.52 (m, 3H), 2.89 (s, 3H), 2.62-2.78 (m, 2H), 2.55 (s, 1H), 2.20-2.42 (m, 2H), 2.12-2.20 (m, 1H), 1.60-1.85 (m, 2H)

### Example 174: (5R,8S)-2-(3-(5-((R)-3-Hydroxy-1-methyl-2-oxopyrrolidin-3-yl)-1H-pyrazol-3-yl)phenyl)-6,7,8,9-tetrahydro-5H-5,8-epoxycyclohepta[d]pyrimidine-4-carboxamide

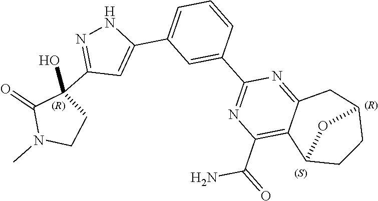

Step A: (5R,8S)-2-(3-Formylphenyl)-6,7,8,9-tetrahydro-5H-5,8-epoxycyclohepta[d]pyrimidine-4-carboxamide. To a solution of (5R,8S)-2-(3-vinylphenyl)-6,7,8,9-tetrahydro-5H-5,8-epoxycyclohepta[d]pyrimidine-4-carboxamide (260 mg, 0.85 mmol, Intermediate 37 Step A) in t-BuOH (6 mL), THF (4 mL) and H₂O (3 mL) was added N-methylmorpholine N-oxide (119 mg, 1.02 mmol) followed by OsO₄ (11 mg, 0.042 mmol). The reaction mixture was stirred at rt for 7 h and then NaHCO₃ (852 mg, 10.151 mmol), NaIO₄ (579 mg, 2.71 mmol) and H₂O (2 mL) were subsequently added. The mixture was stirred for 45 min and then poured into sat. aq. Na₂SO₃. The aqueous phase was extracted with ether, dried (MgSO₄), filtered and concentrated. The residue was purified by prep-TLC (DCM/MeOH=40/1, silica, UV=254 nm) to afford (5R,8S)-2-(3-formylphenyl)-6,7,8,9-tetrahydro-5H-5,8-epoxycyclohepta[d]pyrimidine-4-carboxamide (200 mg, 76.4%) as a white solid. MS (ESI): Mass calcd. for C₁₇H₁₅N₃O₃, 309.3; m/z found, 310.2 [M+H]⁺.

Step B: (5R,8S)-2-(3-(5-((R)-3-hydroxy-1-methyl-2-oxopyrrolidin-3-yl)-1H-pyrazol-3-yl)phenyl)-6,7,8,9-tetrahydro-5H-5,8-epoxycyclohepta[d]pyrimidine-4-carboxamide. To a solution of p-toluenesulfonyl hydrazide (24 mg, 0.13 mmol) in ACN (3 mL), was added (5R,8S)-2-(3-formylphenyl)-6,7,8,9-tetrahydro-5H-5,8-epoxycyclohepta[d]pyrimidine-4-carboxamide (40 mg, 0.13 mmol, Intermediate 37) and the mixture was stirred at rt for 3 h. A solution of aqueous NaOH (5 N, 0.12 mmol) was added. The mixture was stirred for 20 min and (R)-3-ethynyl-3-hydroxy-1-methylpyrrolidin-2-one (Intermediate 1, 90 mg, 0.65 mmol) was added, and the stirring was continued at 50° C. for 16 h. The mixture was concentrated and purified by reverse phase HPLC on a Xtimate 10 m 150 Å, 21.2×250 mm column using a 30 to 60% gradient of ACN/10 mM NH₄HCO₃ and 0.025% NH₃H₂O in H₂O; 10 min gradient to afford (5R,8S)-2-(3-(5-((S)-3-hydroxy-1-methyl-2-oxopyrrolidin-3-yl)-1H-pyrazol-3-yl)phenyl)-6,7,8,9-tetrahydro-5H-5,8-epoxycyclohepta[d]pyrimidine-4-carboxamide (7.2 mg, 11.7%) as an off-white solid. MS (ESI): Mass calcd. For C₂₄H₂₄N₆O₄, 460.485; m/z found, 460.9 [M+H]⁺. ¹H NMR (400 MHz, CDCl₃) δ 8.67 (s, 1H), 8.15 (s, 2H), 7.49 (s, 1H), 7.29-7.40 (m, 1H), 6.61 (s, 1H), 6.32 (s, 1H), 6.11 (s, 1H), 4.85 (s, 1H), 3.30-3.52 (m, 3H), 2.89 (s, 3H), 2.62-2.80 (m, 2H), 2.50-2.60 (m, 1H), 2.30-2.50 (m, 2H), 2.20-2.30 (m, 1H), 1.60-1.80 (m, 2H).

### Example 175: (R)-3-(5-(3-(7-Aminothiazolo[5,4-d]pyrimidin-2-yl)-4-methylphenyl)isoxazol-3-yl)-3-hydroxy-1-methylpyrrolidin-2-one

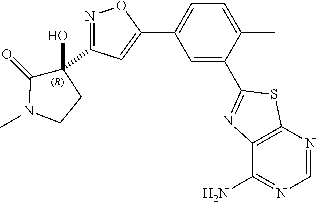

Step A: N-(4,6-Dichloropyrimidin-5-yl)-5-iodo-2-methylbenzamide. A solution of 4,6-dichloropyrimidin-5-amine (1.2 g, 7.49 mmol) in NMP was treated with 5-iodo-2-methylbenzoyl chloride (2.1 g, 7.49 mmol) at 0° C. The reaction mixture was then heated to 70° C. for 16 h, poured into water (50 mL), and the precipitated material was isolated by filtration to afford N-(4,6-dichloropyrimidin-5-yl)-5-iodo-2-methylbenzamide (2.6 g, 85% yield) as a colorless solid. MS (ESI): Mass calcd. For Cl₂H₈Cl₂₁N₃O, 406.91; m/z found, 408 [M+H]⁺.

Step B: 2-(5-Iodo-2-methylphenyl)thiazolo[5,4-d]pyrimidine-7-thiol. A solution of N-(4,6-dichloropyrimidin-5-yl)-5-iodo-2-methylbenzamide (2.20 g, 5.39 mmol) and Lawesson's Reagent (2.18 g, 5.39 mmol) in toluene (60 mL) was heated to reflux for 16 h. The product was isolated by filtration and the solid was washed with toluene (20 mL) to afford 2-(5-iodo-2-methylphenyl)thiazolo[5,4-d]pyrimidine-7-thiol (1.5 g, 72% yield) as a yellow solid. MS (ESI): Mass calcd. For C₁₂H₈₁N₃S₂, 384.92; m/z found, 386 [M+H]⁺.

Step C: 2-(5-Iodo-2-methylphenyl)-7-(methylthio)thiazolo[5,4-d]pyrimidine. A solution of 2-(5-iodo-2-methylphenyl)thiazolo[5,4-d]pyrimidine-7-thiol (1.4 g, 3.63 mmol) and DIEA (1.25 mL, 0.75 g/mL, 7.27 mmol) was treated with methyl iodide (0.68 mL, 2.28 g/mL, 10.90 mmol). The reaction mixture was stirred for 16 h at rt, poured into water (300 mL) and the precipitate was collected by filtration and washed with water (30 mL), followed by MeOH (10 mL) to afford 2-(5-Iodo-2-methylphenyl)-7-(methylthio)thiazolo[5,4-d]pyrimidine (1.0 g, 69% yield) as a yellow solid. MS (ESI): Mass calcd. For C₁₃H₁₀IN₃S₂, 399.27; m/z found, 400 [M+H]⁺.

Step D: Methyl 4-methyl-3-(7-(methylthio)thiazolo[5,4-d]pyrimidin-2-yl)benzoate. A mixture of 2-(5-iodo-2-methylphenyl)-7-(methylthio)thiazolo[5,4-d]pyrimidine (620 mg, 1.55 mmol), Pd(dppf)Cl₂CH₂Cl₂ (63 mg, 0.078 mmol), and TEA (0.43 mL, 0.73 g/mL, 3.11 mmol) in MeOH (40 mL) was heated to 60° C. for 48 h under CO_{(g)}. The mixture was concentrated to dryness and purified by FCC (DCM:MeOH, 20:1) to afford methyl 4-methyl-3-(7-(methylthio)thiazolo[5,4-d]pyrimidin-2-yl)benzoate (350 mg, 68% yield) as a colorless solid. MS (ESI): Mass calcd. For C₁₅H₁₃N₃O₂S₂, 331.04; m/z found, 332 [M+H]⁺.

Step E: 4-Methyl-3-(7-(methylthio)thiazolo[5,4-d]pyrimidin-2-yl)benzoic acid. A solution of methyl 4-methyl-3-(7-(methylthio)thiazolo[5,4-d]pyrimidin-2-yl)benzoate (150 mg, 0.45 mmol) and sodium hydroxide (36 mg, 0.91 mmol) in MeOH (5 mL) and water (2.5 mL) was stirred at rt for 1 h. The reaction mixture was partially concentrated, diluted with water (2 mL), and treated with 1 M HCl_{(aq)} until a precipitate was formed. The solid was filtered and washed with water to afford 4-methyl-3-(7-(methylthio)thiazolo[5,4-d]pyrimidin-2-yl)benzoic acid (140 mg, 71% yield) as a colorless solid. MS (ESI): Mass calcd. For C₁₄H₁₁N₃O₂S₂, 317.03; m/z found, 318.0 [M+H]⁺.

Step F: 4-Methyl-3-(7-(methylthio)thiazolo[5,4-d]pyrimidin-2-yl)benzoyl chloride. A solution of 4-methyl-3-(7-(methylthio)thiazolo[5,4-d]pyrimidin-2-yl)benzoic acid (140 mg, 0.44 mmol) in thionyl chloride (3 mL, 1.64 g/mL, 41.36 mmol) was heated to 80° C. for 2 h. The reaction mixture was concentrated to dryness to afford 4-methyl-3-(7-(methylthio)thiazolo[5,4-d]pyrimidin-2-yl)benzoyl chloride as a yellow oil. MS (ESI): Mass calcd. For C₁₄H₁₀ClN₃OS₂, 334.00; m/z found, 332.0 [M−Cl+OH]⁺.

Step G: (R)-3-Hydroxy-1-methyl-3-(3-(4-methyl-3-(7-(methylthio)thiazolo[5,4-d]pyrimidin-2-yl)phenyl)-3-oxoprop-1-yn-1-yl)pyrrolidin-2-one. To a solution of (R)-3-ethynyl-3-hydroxy-1-methylpyrrolidin-2-one (Intermediate 1, 46 mg, 0.33 mmol) at −20° C., was added bis(triphenylphosphine)palladium(II) dichloride (9.2 mg, 0.13 mmol) followed by copper(I)iodide (3.1 mg, 0.016 mmol) and TEA. After 5 min, a solution of 4-methyl-3-(7-(methylthio)thiazolo[5,4-d]pyrimidin-2-yl)benzoyl chloride (110 mg, 0.33 mmol) in THF (5 mL) was added dropwise and the mixture was allowed to warm to 0° C. and stirred for 2 h. The reaction mixture was diluted with water (20 mL) and extracted with EtOAc (3×20 mL). The combined organic layers were washed with brine (30 mL), dried over Na₂SO₄, filtered, concentrated and the residue was purified by FCC (DCM:MeOH, 100:1) to afford (R)-3-hydroxy-1-methyl-3-(3-(4-methyl-3-(7-(methylthio)thiazolo[5,4-d]pyrimidin-2-yl)phenyl)-3-oxoprop-1-yn-1-yl)pyrrolidin-2-one (500 mg, 81% yield) as a yellow solid. MS (ESI): Mass calcd. For C₂₁H₁₈N₄O₃S₂, 438.08; m/z found, 439.0 [M+H]⁺.

Step H. (R)-3-Hydroxy-1-methyl-3-(5-(4-methyl-3-(7-(methylthio)thiazolo[5,4-d]pyrimidin-2-yl)phenyl)isoxazol-3-yl)pyrrolidin-2-one. A solution of (R)-3-hydroxy-1-methyl-3-(3-(4-methyl-3-(7-(methylthio)thiazolo[5,4-d]pyrimidin-2-yl)phenyl)-3-oxoprop-1-yn-1-yl)pyrrolidin-2-one (50 mg, 0.11 mmol) and sodium azide (37 mg, 0.57 mmol) in DCM (2 mL) was cooled to 0° C. and treated with AcOH (0.033 mL, 0.57 mmol) followed by TEA (0.003 mL, 0.02 mmol). The reaction mixture was allowed to warm to rt, stirred for 16 h and then treated with sat. aq. NaHCO₃ (5 mL) for 10 min. The organic layer was separated and the aqueous layer was further extracted with DCM (2×10 mL). The combined organic layers were dried (Na₂SO₄), filtered and concentrated using a stream of N₂ and purified by FCC (DCM:MeOH, 50:1) to afford (R)-3-hydroxy-1-methyl-3-(5-(4-methyl-3-(7-(methylthio)thiazolo[5,4-d]pyrimidin-2-yl)phenyl)isoxazol-3-yl)pyrrolidin-2-one (30 mg, 73% yield) as a yellow oil. MS (ESI): Mass calcd. For C₂₁H₁₉N₅O₃S₂, 453.09; m/z found, 454.0 [M+H]⁺.

Step I. (R)-3-(5-(3-(7-aminothiazolo[5,4-d]pyrimidin-2-yl)-4-methylphenyl)isoxazol-3-yl)-3-hydroxy-1-methylpyrrolidin-2-one. A solution of (R)-3-hydroxy-1-methyl-3-(5-(4-methyl-3-(7-(methylthio)thiazolo[5,4-d]pyrimidin-2-yl)phenyl)isoxazol-3-yl)pyrrolidin-2-one (30 mg, 0.066 mmol) in NH₃ in MeOH (5 mL, 7 M, 35 mmol) was heated to 75° C. for 48 h in a sealed tube. The reaction mixture was cooled to rt, concentrated and purified by reverse phase HPLC Xtimate C18 2×250 mm 10 μm using mobile phase A: 10 mM NH₄CO₃ in water+0.1% 0.025% NH₄OH and B: ACN with a gradient 5% B for 3 min then 5-40% B over 7 min at 30 mL/min to afford (R)-3-(5-(3-(7-aminothiazolo[5,4-d]pyrimidin-2-yl)-4-methylphenyl)isoxazol-3-yl)-3-hydroxy-1-methylpyrrolidin-2-one (3.3 mg, 11% yield) as a colorless solid. MS (ESI): Mass calcd. for C₂₃H₁₈FN₅O₄, 447.42; m/z found, 448.2 [M+H]⁺. ¹H NMR (400 MHz, DMSO-d₆) δ 8.36 (s, 1H), 8.26 (d, J=2 Hz, 1H), 7.96 (dd, J=1.6, 8.0 Hz, 1H), 7.83 (s, 2H), 7.61 (d, J=8 Hz, 1H), 7.19 (s, 1H), 6.51 (s, 1H), 3.38-3.45 (m, 2H), 2.81 (s, 3H), 2.69 (s, 3H), 2.63-2.67 (m, 1H), 2.23-2.27 (m, 1H).

### Example 176: (R,S)-3-(5-(3-((7-Aminothiazolo[5,4-d]pyrimidin-2-yl)amino)phenyl)isoxazol-3-yl)-3-hydroxy-1-methylpyrrolidin-2-one

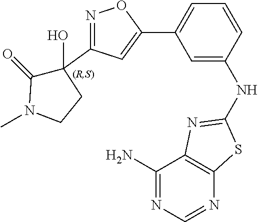

Step A: (R,S)-3-Hydroxy-3-(5-(3-isothiocyanatophenyl)isoxazol-3-yl)-1-methylpyrrolidin-2-one. To a solution of (R,S)-3-(5-(3-aminophenyl)isoxazol-3-yl)-3-hydroxy-1-methylpyrrolidin-2-one (60 mg, 0.22 mmol, Intermediate 35) in THF (10 mL) at 0° C., was added ice (100 mg), followed by thiophosgene (0.034 mL, 0.44 mmol). The reaction mixture was stirred at 0° C. for 2 h, transferred to a separatory funnel and washed with water (2×15 mL) followed by sat. aq. NaHCO₃ (15 mL). The organic layer was dried (Na₂SO₄), filtered and concentrated at 20° C. to afford (R,S)-3-hydroxy-3-(5-(3-isothiocyanatophenyl)isoxazol-3-yl)-1-methylpyrrolidin-2-one (65 mg, 94% yield) as a yellow oil. MS (ESI): Mass calcd. for C₁₅H₁₃N₃O₃S, 315.07; m/z found, 316.1 [M+H]⁺.

Step B: (R,S)-3-(5-(3-((7-Chlorothiazolo[5,4-d]pyrimidin-2-yl)amino)phenyl)isoxazol-3-yl)-3-hydroxy-1-methylpyrrolidin-2-one. To a solution of (R,S)-3-hydroxy-3-(5-(3-isothiocyanatophenyl)isoxazol-3-yl)-1-methylpyrrolidin-2-one (65 mg, 0.21 mmol) in ACN (6 mL) was added 4,6-dichloropyrimidin-5-amine (100 mg, 0.62 mmol), followed by Cs₂CO₃ (269 mg, 0.82 mmol). The reaction mixture was heated to 50° C. for 2 h, cooled to rt and the solid was removed by filtration. The filter cake was washed with ACN (5 mL) and the filtrate was concentrated and purified by prep-TLC (DCM:MeOH, 20:1) to afford (R,S)-3-(5-(3-((7-chlorothiazolo[5,4-d]pyrimidin-2-yl)amino)phenyl)isoxazol-3-yl)-3-hydroxy-1-methylpyrrolidin-2-one (30 mg, 33% yield) as a yellow oil. MS (ESI): Mass calcd. for C₁₉H₁₅ClN₆O₃S, 442.06; m/z found, 442.9 [M+H]⁺.

Step C. (R,S)-3-(5-(3-((7-Aminothiazolo[5,4-d]pyrimidin-2-yl)amino)phenyl)isoxazol-3-yl)-3-hydroxy-1-methylpyrrolidin-2-one. A solution of (R,S)-3-(5-(3-((7-chlorothiazolo[5,4-d]pyrimidin-2-yl)amino)phenyl)isoxazol-3-yl)-3-hydroxy-1-methylpyrrolidin-2-one (30 mg, 0.067 mmol) in NH₃ in EtOH (5 mL, 7 M, 35 mmol) was heated to 75° C. for 16 h in a sealed tube. The reaction mixture was cooled to rt, concentrated and purified by reverse phase HPLC Xtimate C18 2×250 mm 10 μm using mobile phase A: 10 mM NH₄CO₃ in water+0.1% (0.025% NH₄OH) and B: ACN with a gradient 5% B for 3 min then 5-40% B over 7 min at 30 mL/min to afford (R,S)-3-(5-(3-((7-aminothiazolo[5,4-d]pyrimidin-2-yl)amino)phenyl)isoxazol-3-yl)-3-hydroxy-1-methylpyrrolidin-2-one (5.1 mg, 18% yield) as a colorless solid. MS (ESI): Mass calcd. for C₁₉H₁₇N₇O₃S, 423.46; m/z found, 424.2 [M+H]⁺. ¹H NMR (400 MHz, DMSO-d₆) δ 10.78 (s, 1H), 8.40 (d, J=6.8 Hz, 1H), 8.14 (s, 1H), 7.87 (s, 1H), 7.48-7.52 (m, 2H), 7.18 (br. s, 2H), 7.06 (s, 1H), 6.51 (s, 1H), 3.36-3.48 (m, 2H), 2.81 (s, 3H), 2.64-2.70 (m, 1H), 2.20-2.28 (m, 1H).

### Example 177: (R)-3-Amino-4-cyclopropyl-6-(3-(5-(3-hydroxy-1-methyl-2-oxopyrrolidin-3-yl-4,4,5,5-d₄)isoxazol-3-yl)phenyl)picolinamide

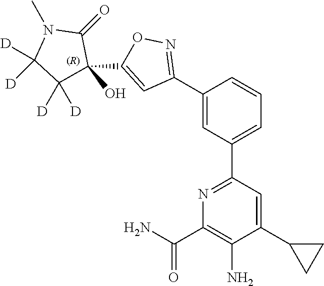

(R)-3-Amino-4-cyclopropyl-6-(3-(5-(3-hydroxy-1-methyl-2-oxopyrrolidin-3-yl-4,4,5,5-d₄)isoxazol-3-yl)phenyl)picolinamide (27 mg) was prepared in a manner analogous to Example 23, using (R)-3-hydroxy-1-methyl-3-(3-(3-(4,4,5,5-tetramethyl-1,3,2-dioxaborolan-2-yl)phenyl)isoxazol-5-yl)pyrrolidin-2-one-4,4,5,5-d₄ (Intermediate 47) and 3-amino-6-chloro-4-cyclopropylpicolinamide (Example 88 Step A) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 1-bromoimidazo[1,5-a]pyridine-3-carboxamide. MS (ESI): Mass calcd. for C₂₃H₂₃N₅O₄ 437.49 m/z found 438.30 [M+H]⁺. ¹H NMR (400 MHz, CD₃OD) δ 8.44 (t, J=1.8 Hz, 1H), 8.08 (dt, J=8.1, 1.3 Hz, 1H), 7.77 (dt, J=7.7, 1.3 Hz, 1H), 7.63 (d, J=0.9 Hz, 1H), 7.54 (t, J=7.8 Hz, 1H), 7.00 (s, 1H), 2.96 (s, 3H), 1.88-1.71 (m, 1H), 1.18-1.00 (m, 2H), 0.81-0.67 (m, 2H).

### Example 178: 3-Amino-4-cyclopropyl-6-(3-(5-((3R,5S)-3-hydroxy-1-methyl-2-oxo-5-(trifluoromethyl)pyrrolidin-3-yl)isoxazol-3-yl)phenyl)picolinamide

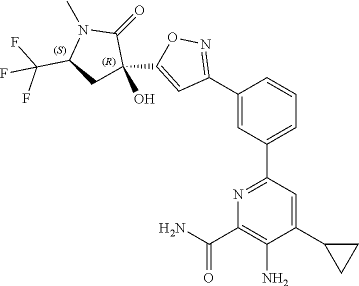

3-Amino-4-cyclopropyl-6-(3-(5-((3R,5S)-3-hydroxy-1-methyl-2-oxo-5-(trifluoromethyl)pyrrolidin-3-yl)isoxazol-3-yl)phenyl)picolinamide (105 mg) was prepared in a manner analogous to Example 23, using (3R,5S)-3-hydroxy-1-methyl-3-(3-(3-(4,4,5,5-tetramethyl-1,3,2-dioxaborolan-2-yl)phenyl)isoxazol-5-yl)-5-(trifluoromethyl)pyrrolidin-2-one (Intermediate 48) and 3-amino-6-chloro-4-cyclopropylpicolinamide (Example 88 Step A) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 1-bromoimidazo[1,5-a]pyridine-3-carboxamide. MS (ESI): Mass calcd for C₂₄H₂₂F₃N₅O₄ 501.46 m/z found 502.20 [M+H]⁺. ¹H NMR (400 MHz, Chloroform-d) δ 8.15 (t, J=1.8 Hz, 1H), 8.07 (br s, 1H), 7.93-7.81 (m, 1H), 7.58-7.62 (m, 1H), 7.48-7.36 (m, 2H), 6.78 (s, 1H), 6.43 (br s, 2H), 5.73 (d, J=4.5 Hz, 1H), 4.73 (br s, 1H), 4.38-4.17 (m, 1H), 3.10 (s, 3H), 3.02 (dd, J=14.1, 7.8 Hz, 1H), 2.51 (dd, J=14.2, 6.8 Hz, 1H), 1.76-1.58 (m, 1H), 1.16-0.97 (m, 2H), 0.81-0.59 (m, 2H).

### Example 179: (R)-3-Amino-4-cyclopropyl-6-(3-(5-(3-hydroxy-1-(methyl-d₃)-2-oxopyrrolidin-3-yl)isoxazol-3-yl)phenyl)picolinamide

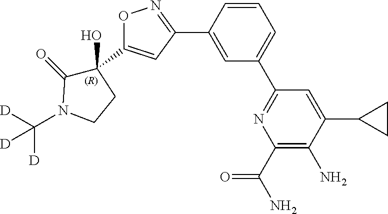

(R)-3-Amino-4-cyclopropyl-6-(3-(5-(3-hydroxy-1-(methyl-d₃)-2-oxopyrrolidin-3-yl)isoxazol-3-yl)phenyl)picolinamide (131 mg) was prepared in a manner analogous to Example 23, using (R)-3-hydroxy-1-(methyl-d₃)-3-(3-(3-(4,4,5,5-tetramethyl-1,3,2-dioxaborolan-2-yl)phenyl)isoxazol-5-yl)pyrrolidin-2-one (Intermediate 50) and 3-amino-6-chloro-4-cyclopropylpicolinamide (Example 88 Step A) instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 1-bromoimidazo[1,5-a]pyridine-3-carboxamide. MS (ESI): Mass calcd. for C₂₃H₂₃N₅O₄ 436.49 m/z found 437.30 [M+H]⁺. ¹H NMR (400 MHz, CD₃OD) δ 8.44 (br s, 1H), 8.13-8.01 (m, 1H), 7.74-7.78 (m, 1H), 7.62 (br s, 1H), 7.54 (t, J=7.8 Hz, 1H), 7.00 (s, 1H), 3.64-3.49 (m, 2H), 2.72-2.78 (m, 1H), 2.37-2.44 (m, 1H), 1.89-1.69 (m, 1H), 1.11-1.04 (m, 2H), 0.86-0.69 (m, 2H).

### Example 180: (R)-1-(3-(5-(3-Hydroxy-1-methyl-2-oxopyrrolidin-3-yl-4,4,5,5-d₄)isoxazol-3-yl)phenyl)-5,6,7,8-tetrahydroimidazo[1,5-a]pyridine-3-carboxamide

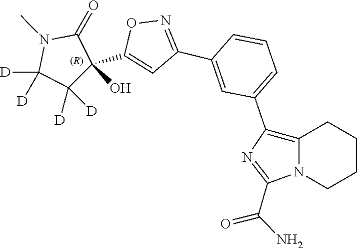

(R)-1-(3-(5-(3-Hydroxy-1-methyl-2-oxopyrrolidin-3-yl-4,4,5,5-d₄)isoxazol-3-yl)phenyl)-5,6,7,8-tetrahydroimidazo[1,5-a]pyridine-3-carboxamide (21 mg) was prepared in a manner analogous to Example 89 using (R)-3-hydroxy-1-methyl-3-(3-(3-(4,4,5,5-tetramethyl-1,3,2-dioxaborolan-2-yl)phenyl)isoxazol-5-yl)pyrrolidin-2-one-4,4,5,5-d₄ (Intermediate 47) and 1-bromo-5,6,7,8-tetrahydroimidazo[1,5-a]pyridine-3-carboxylic acid instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 3-amino-6-bromo-5-fluoropicolinic acid. MS (ESI): Mass calcd. for C₂₃H₂₃N₅O₄ 436.49 m/z found 437.30 [M+H]⁺. ¹H NMR (400 MHz, Chloroform-d) δ 8.04 (t, J=1.7 Hz, 1H), 7.68-7.71 (m, 1H), 7.64-7.56 (m, 1H), 7.47-7.30 (m, 2H), 6.75 (s, 1H), 5.78 (s, 1H), 4.96 (s, 1H), 4.44-4.51 (m, 2H), 3.07-2.85 (m, 6H), 2.04-1.90 (m, 2H), 1.90-1.76 (m, 2H).

### Example 181: (R)-1-(3-(5-(3-Hydroxy-1-(methyl-d₃)-2-oxopyrrolidin-3-yl)isoxazol-3-yl)phenyl)-5,6,7,8-tetrahydroimidazo[1,5-a]pyridine-3-carboxamide

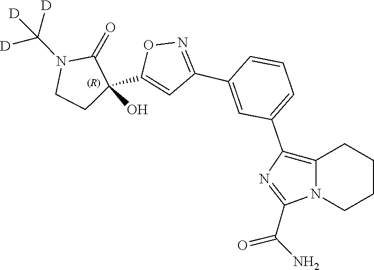

(R)-1-(3-(5-(3-Hydroxy-1-(methyl-d₃)-2-oxopyrrolidin-3-yl)isoxazol-3-yl)phenyl)-5,6,7,8-tetrahydroimidazo[1,5-a]pyridine-3-carboxamide (21 mg) was prepared in a manner analogous to Example 89, using (R)-3-hydroxy-1-(methyl-d₃)-3-(3-(3-(4,4,5,5-tetramethyl-1,3,2-dioxaborolan-2-yl)phenyl)isoxazol-5-yl)pyrrolidin-2-one (Intermediate 50) and 1-bromo-5,6,7,8-tetrahydroimidazo[1,5-a]pyridine-3-carboxylic acid instead of (R)-3-hydroxy-1-methyl-3-(3-(3-(4,4,5,5-tetramethyl-1,3,2-dioxaborolan-2-yl)phenyl)isoxazol-5-yl)pyrrolidin-2-one and 3-amino-6-bromo-5-fluoropicolinic acid. MS (ESI): Mass calcd. for C₂₂H₂₃N₅O₄ 424.47 m/z found 425.30 [M+H]⁺. ¹H NMR (500 MHz, Chloroform-d) δ 8.08 (br s, 1H), 7.73 (dt, J=7.8, 1.4 Hz, 1H), 7.63 (dt, J=7.9, 1.3 Hz, 1H), 7.43 (t, J=7.8 Hz, 1H), 7.35-7.28 (m, 1H), 6.75 (s, 1H), 5.56 (br s, 1H), 4.58-4.46 (m, 2H), 4.39 (br s, 1H), 3.60-3.65 (m, 1H), 3.44-3.51 (m, 1H), 3.07-2.91 (m, 4H), 2.78-2.82 (m, 1H), 2.42-2.50 (m, 1H), 2.05-1.94 (m, 2H), 1.84-1.90 (m, 2H).

### Example 182: (R)-6-(3-(5-(3-Hydroxy-1-methyl-2-oxopyrrolidin-3-yl)isoxazol-3-yl)phenyl)-4-(1-methyl-1H-pyrazol-5-yl)picolinamide

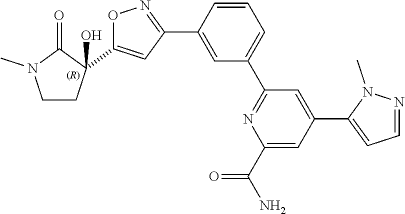

(R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 162 mg, 0.42 mmol), 6-chloro-4-(1-methyl-1H-pyrazol-5-yl)picolinamide (Intermediate 52, 100 mg, 0.42 mmol), Pd(dtbpf)Cl₂ (41 mg, 0.063 mmol), K₃PO₄ (366 mg, 1.69 mmol), 1,4-dioxane (2.1 mL), and H₂O (0.5 mL) were added to a 5 mL vial, which was subsequently evacuated and refilled with Ar (×3), and stirred for 3 h at 85° C. The resulting residue was purified by preparative HPLC (XBridge OBD C18 5 μm, 50×100 mm column using a 0 to 95% gradient of ACN/20 mM NH₄OH in H₂O over 16 min. Detection, UV at 25° C.=220-254 nM) to yield a yellow solid (24 mg, 12%). MS (ESI): Mass calcd. for C₂₄H₂₂N₆O₄ 458.2 m/z, found 459.2 [M+H]⁺. ¹H NMR (600 MHz, CD₃OD) δ 8.67 (t, J=1.8 Hz, 1H), 8.31 (ddd, J=7.9, 1.8, 1.1 Hz, 1H), 8.20 (dd, J=23.1, 1.5 Hz, 2H), 7.93 (dt, J=7.7, 1.3 Hz, 1H), 7.64 (t, J=7.8 Hz, 1H), 7.60 (d, J=2.0 Hz, 1H), 7.06 (s, 1H), 6.71 (d, J=2.0 Hz, 1H), 4.04 (s, 3H), 3.66-3.54 (m, 2H), 2.99 (s, 3H), 2.76 (ddd, J=13.6, 7.2, 4.7 Hz, 1H), 2.43 (ddd, J=13.6, 8.1, 6.4 Hz, 1H).

### Example 183: (R)-6-(3-(5-(3-Hydroxy-1-methyl-2-oxopyrrolidin-3-yl)isoxazol-3-yl)phenyl)-4-(isothiazol-5-yl)picolinamide

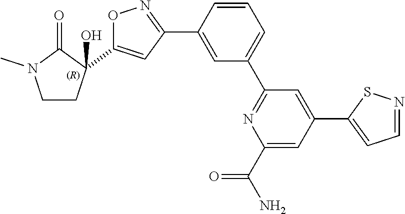

The title compound (60 mg, 59%) was prepared with analogous conditions to those described in Example 182 using 6-chloro-4-(isothiazol-5-yl)picolinamide in place of 6-chloro-4-(1-methyl-1H-pyrazol-5-yl)picolinamide. MS (ESI): Mass calcd. for C₂₃H₁₉N₅O₄S 461.1 m/z, found 462.3 [M+H]⁺. ¹H NMR (600 MHz, CD₃OD) δ 8.75 (t, J=1.8 Hz, 1H), 8.65 (d, J=1.8 Hz, 1H), 8.45 (d, J=1.5 Hz, 1H), 8.40 (ddd, J=7.9, 1.9, 1.0 Hz, 1H), 8.36 (d, J=1.6 Hz, 1H), 8.04 (d, J=1.8 Hz, 1H), 8.00 (dt, J=7.8, 1.2 Hz, 1H), 7.70 (t, J=7.8 Hz, 1H), 7.11 (s, 1H), 3.67-3.57 (m, 2H), 3.00 (s, 3H), 2.84-2.73 (m, 1H), 2.44 (ddd, J=13.6, 8.0, 6.5 Hz, 1H).

### Example 184: (R)-6-(3-(5-(3-Hydroxy-1-methyl-2-oxopyrrolidin-3-yl)isoxazol-3-yl)phenyl)-4-(thiazol-5-yl)picolinamide

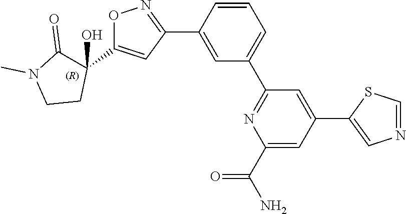

The title compound (60 mg, 59%) was prepared with analogous conditions to those described in Example 182 using 6-chloro-4-(isothiazol-5-yl)picolinamide in place of 6-chloro-4-(1-methyl-1H-pyrazol-5-yl)picolinamide. MS (ESI): Mass calcd. for C₂₃H₁₉N₅O₄S 461.1 m/z, found 462.3 [M+H]⁺. ¹H NMR (600 MHz, CD₃OD) δ 8.75 (t, J=1.8 Hz, 1H), 8.65 (d, J=1.8 Hz, 1H), 8.45 (d, J=1.5 Hz, 1H), 8.40 (ddd, J=7.9, 1.9, 1.0 Hz, 1H), 8.36 (d, J=1.6 Hz, 1H), 8.04 (d, J=1.8 Hz, 1H), 8.00 (dt, J=7.8, 1.2 Hz, 1H), 7.70 (t, J=7.8 Hz, 1H), 7.11 (s, 1H), 3.67-3.57 (m, 2H), 3.00 (s, 3H), 2.84-2.73 (m, 1H), 2.44 (ddd, J=13.6, 8.0, 6.5 Hz, 1H).

### Example 185: (R)-4-(1-Cyclopropyl-1H-pyrazol-5-yl)-6-(3-(5-(3-hydroxy-1-methyl-2-oxopyrrolidin-3-yl)isoxazol-3-yl)phenyl)picolinamide

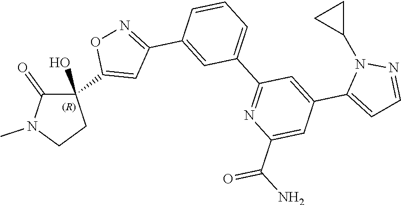

To a mixture of (R)-4-chloro-6-(3-(5-(3-hydroxy-1-methyl-2-oxopyrrolidin-3-yl)isoxazol-3-yl)phenyl)picolinamide (Intermediate 57, 50 mg, 0.12 mmol) and 1-cyclopropyl-5-(4,4,5,5-tetramethyl-1,3,2-dioxaborolan-2-yl)-1H-pyrazole (51 mg, 0.12 mmol) in 1,4-dioxane (1.2 mL) and water (0.24 mL) were added K₃PO₄ (79 mg, 0.36 mmol) and Pd(dppf)Cl₂ (13 mg, 0.021 mmol) and the resulting mixture was degassed and purged with N₂ 3 times, and then the mixture was heated at 100° C. for 2 h under a N₂ atmosphere. The reaction mixture was filtered through a syringe filter. The resulting solution was purified by preparative HPLC (Waters XBridge BEH C18, 5 m, 30×150 mm using a 10 to 45% gradient of ACN/20 mM NH₄OH in H₂O over 20 min. Detection, UV at 25° C.=220-254 nM) to yield an orange residue (31 mg, 53%). MS (ESI): Mass calcd. for C₂₆H₂₄N₆O₄ 484.2 m/z, found 485.2 [M+H]⁺. ¹H NMR (400 MHz, CD₃OD) δ 8.76 (t, J=1.7 Hz, 1H), 8.44 (dd, J=9.5, 1.5 Hz, 2H), 8.38 (ddd, J=7.9, 1.9, 1.1 Hz, 1H), 7.99 (ddd, J=7.8, 1.8, 1.1 Hz, 1H), 7.70 (t, J=7.8 Hz, 1H), 7.59 (d, J=2.0 Hz, 1H), 7.09 (s, 1H), 6.79 (d, J=2.0 Hz, 1H), 3.97-3.89 (m, 1H), 3.66-3.53 (m, 2H), 2.99 (s, 3H), 2.78 (ddd, J=13.6, 6.8, 5.1 Hz, 1H), 2.43 (ddd, J=13.5, 7.9, 6.7 Hz, 1H), 1.18-1.04 (m, 4H).

### Example 186: (R)-6-(3-(5-(3-Hydroxy-1-methyl-2-oxopyrrolidin-3-yl)isoxazol-3-yl)phenyl)-4-(2-methyloxazol-5-yl)picolinamide

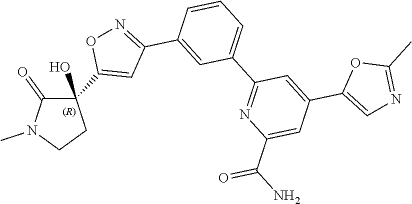

The title compound as its TFA salt was prepared with analogous conditions to those described in Example 185 using 2-methyl-5-(4,4,5,5-tetramethyl-1,3,2-dioxaborolan-2-yl)oxazole in place of 1-cyclopropyl-5-(4,4,5,5-tetramethyl-1,3,2-dioxaborolan-2-yl)-1H-pyrazole. The resulting solution was purified by preparative HPLC (Waters XSelect CSH C18, 5 m, 30×150 mm using a 10 to 45% gradient of ACN/0.16% TFA in H₂O/0.16% TFA over 15 min. Detection, UV at 25° C.=220-254 nM) to yield an orange residue as a TFA salt (25 mg, 35%). MS (ESI): Mass calcd. for C₂₄H₂₁N₅O₅ 459.2 m/z, found 460.2 [M+H]⁺. ¹H NMR (400 MHz, CD₃OD) δ 8.71 (t, J=1.6 Hz, 1H), 8.39-8.32 (m, 3H), 7.99-7.94 (m, 1H), 7.88 (s, 1H), 7.68 (t, J=7.8 Hz, 1H), 7.09 (s, 1H), 3.64-3.57 (m, 2H), 3.00 (s, 3H), 2.79 (ddd, J=13.6, 6.8, 5.2 Hz, 1H), 2.63 (s, 3H), 2.44 (ddd, J=13.5, 7.8, 6.7 Hz, 1H).

### Example 187: (R)-4-(1-Ethyl-3-methyl-1H-pyrazol-4-yl)-6-(3-(5-(3-hydroxy-1-methyl-2-oxopyrrolidin-3-yl)isoxazol-3-yl)phenyl)picolinamide

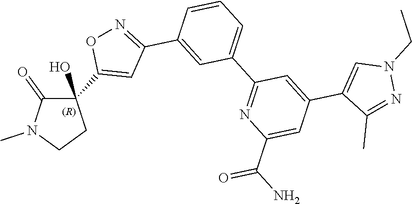

The title compound as its TFA salt was prepared with analogous conditions to those described in Example 185 using 1-ethyl-3-methyl-4-(4,4,5,5-tetramethyl-1,3,2-dioxaborolan-2-yl)-1H-pyrazole in place of 1-cyclopropyl-5-(4,4,5,5-tetramethyl-1,3,2-dioxaborolan-2-yl)-1H-pyrazole. The resulting solution was purified by preparative HPLC (Waters XSelect CSH C18, 5 m, 30×150 mm using a 10 to 45% gradient of ACN/0.16% TFA in H₂O/0.16% TFA over 15 min. Detection, UV at 25° C.=220-254 nM) to yield an orange residue as a TFA salt (49 mg, 67%). MS (ESI): Mass calcd. for C₂₆H₂₆N₆O₄ 486.2 m/z, found 487.2 [M+H]⁺. ¹H NMR (400 MHz, CD₃OD) δ 8.68 (t, J=1.7 Hz, 1H), 8.31 (ddd, J=7.9, 1.9, 1.1 Hz, 1H), 8.24 (s, 1H), 8.19 (dd, J=11.1, 1.6 Hz, 2H), 7.95 (ddd, J=7.8, 1.8, 1.1 Hz, 1H), 7.67 (t, J=7.8 Hz, 1H), 7.07 (s, 1H), 4.22 (q, J=7.3 Hz, 2H), 3.64-3.58 (m, 2H), 3.00 (s, 3H), 2.78 (ddd, J=13.5, 6.8, 5.1 Hz, 1H), 2.55 (s, 3H), 2.44 (ddd, J=13.5, 7.8, 6.7 Hz, 1H), 1.53 (t, J=7.3 Hz, 3H).

### Example 188: (R)-4-(1-Ethyl-3-(trifluoromethyl)-1H-pyrazol-5-yl)-6-(3-(5-(3-hydroxy-1-methyl-2-oxopyrrolidin-3-yl)isoxazol-3-yl)phenyl)picolinamide

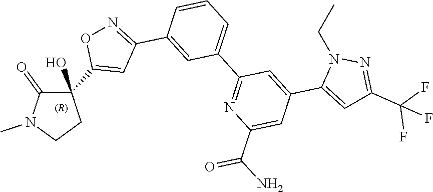

The title compound was prepared with analogous conditions to those described in Example 185 using (1-ethyl-3-(trifluoromethyl)-1H-pyrazol-5-yl)boronic acid in place of 1-cyclopropyl-5-(4,4,5,5-tetramethyl-1,3,2-dioxaborolan-2-yl)-1H-pyrazole. The resulting solution was purified by preparative HPLC (Waters XBridge BEH C18, 5 m, 30×150 mm using a 30 to 65% gradient of ACN/20 mM NH₄OH in H₂O over 15 min. Detection, UV at 25° C.=220-254 nM) to yield an orange residue (46 mg, 70%). MS (ESI): Mass calcd. for C₂₆H₂₃F₃N₆O₄ 540.2 m/z, found 541.3 [M+H]⁺. ¹H NMR (400 MHz, CD₃OD) δ 8.73 (t, J=1.7 Hz, 1H), 8.36 (ddd, J=8.0, 1.9, 1.1 Hz, 1H), 8.29 (d, J=1.5 Hz, 1H), 8.20 (d, J=1.5 Hz, 1H), 7.97 (dt, J=7.8, 1.3 Hz, 1H), 7.67 (t, J=7.8 Hz, 1H), 7.08 (s, 1H), 7.02 (s, 1H), 4.39 (q, J=7.2 Hz, 2H), 3.63-3.56 (m, 2H), 2.99 (s, 3H), 2.77 (ddd, J=13.5, 6.9, 5.0 Hz, 1H), 2.43 (ddd, J=13.5, 7.9, 6.5 Hz, 1H), 1.50 (t, J=7.2 Hz, 3H).

### Example 189: (R)-6-(3-(5-(3-Hydroxy-1-methyl-2-oxopyrrolidin-3-yl)isoxazol-3-yl)phenyl)-4-(1-(2-methoxyethyl)-1H-pyrazol-4-yl)picolinamide

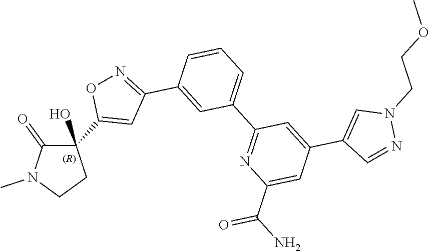

The title compound as its TFA salt was prepared with analogous conditions to those described in Example 185 using 1-(2-methoxyethyl)-4-(4,4,5,5-tetramethyl-1,3,2-dioxaborolan-2-yl)-1H-pyrazole in place of 1-cyclopropyl-5-(4,4,5,5-tetramethyl-1,3,2-dioxaborolan-2-yl)-1H-pyrazole. The resulting solution was purified by preparative HPLC (Waters XSelect CSH C18, 5 m, 30×150 mm using a 10 to 45% gradient of ACN/0.16% TFA in H₂O/0.16% TFA over 15 min. Detection, UV at 25° C.=220-254 nM) to yield an orange residue as a TFA salt (50 mg, 67%). MS (ESI): Mass calcd. for C₂₆H₂₆N₆O₅ 502.2 m/z, found 503.3 [M+H]⁺. ¹H NMR (400 MHz, CD₃OD) δ 8.68 (t, J=1.7 Hz, 1H), 8.31 (ddd, J=7.9, 1.9, 1.1 Hz, 1H), 8.24 (s, 1H), 8.19 (dd, J=11.1, 1.6 Hz, 2H), 7.95 (ddd, J=7.8, 1.8, 1.1 Hz, 1H), 7.67 (t, J=7.8 Hz, 1H), 7.07 (s, 1H), 4.22 (q, J=7.3 Hz, 2H), 3.64-3.58 (m, 2H), 3.00 (s, 3H), 2.78 (ddd, J=13.5, 6.8, 5.1 Hz, 1H), 2.55 (s, 3H), 2.44 (ddd, J=13.5, 7.8, 6.7 Hz, 1H), 1.53 (t, J=7.3 Hz, 3H).

### Example 190: (R)-4-(3-Cyclopropyl-1H-pyrazol-4-yl)-6-(3-(5-(3-hydroxy-1-methyl-2-oxopyrrolidin-3-yl)isoxazol-3-yl)phenyl)picolinamide

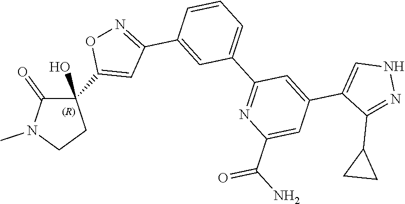

The title compound was prepared with analogous conditions to those described in Example 185 using 3-cyclopropyl-4-(4,4,5,5-tetramethyl-1,3,2-dioxaborolan-2-yl)-1H-pyrazole in place of 1-cyclopropyl-5-(4,4,5,5-tetramethyl-1,3,2-dioxaborolan-2-yl)-1H-pyrazole. The resulting solution was purified by preparative HPLC (Waters XBridge BEH C18, 5 m, 30×150 mm using a 10 to 45% gradient of ACN/20 mM NH₄OH in H₂O over 15 min. Detection, UV at 25° C.=220-254 nM) to yield an orange residue (9 mg, 15%). MS (ESI): Mass calcd. for C₂₆H₂₄N₆O₄ 484.2 m/z, found 485.4 [M+H]⁺. ¹H NMR (400 MHz, CD₃OD) δ 8.71 (t, J=1.7 Hz, 1H), 8.45 (d, J=1.6 Hz, 1H), 8.40 (d, J=1.6 Hz, 1H), 8.33 (ddd, J=7.9, 1.9, 1.1 Hz, 1H), 7.95 (ddd, J=7.7, 1.8, 1.1 Hz, 1H), 7.67 (t, J=7.8 Hz, 1H), 7.07 (s, 1H), 3.67-3.56 (m, 2H), 3.00 (s, 3H), 2.78 (ddd, J=13.5, 6.7, 5.2 Hz, 1H), 2.44 (ddd, J=13.5, 7.8, 6.7 Hz, 1H).

### Example 191: (R)-6-(3-(5-(3-Hydroxy-1-methyl-2-oxopyrrolidin-3-yl)isoxazol-3-yl)phenyl)-4-(2-methoxythiazol-5-yl)picolinamide

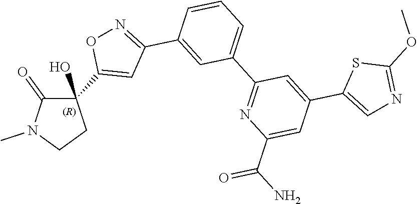

The title compound was prepared with analogous conditions described in Example 185 using 2-methoxy-5-(4,4,5,5-tetramethyl-1,3,2-dioxaborolan-2-yl)thiazole in place of 1-cyclopropyl-5-(4,4,5,5-tetramethyl-1,3,2-dioxaborolan-2-yl)-1H-pyrazole. The resulting solution was purified by preparative HPLC (Waters XBridge BEH C18, 5 m, 30×150 mm using a 10 to 45% gradient of ACN/20 mM NH₄OH in H₂O over 15 min. Detection, UV at 25° C.=220-254 nM) to yield a yellow residue (6 mg, 10%). MS (ESI): Mass calcd. for C₂₄H₂₁N₅O₅S 491.1 m/z, found 492.3 [M+H]⁺. ¹H NMR (400 MHz, CD₃OD) δ 8.76-8.68 (m, 1H), 8.32-8.25 (m, 1H), 8.20-8.14 (m, 1H), 8.04-7.95 (m, 2H), 7.74-7.65 (m, 1H), 7.13-7.05 (m, 1H), 4.19 (s, 2H), 3.66-3.56 (m, 3H), 3.00 (s, 3H), 2.81-2.73 (m, 2H), 2.48-2.37 (m, 2H).

### Example 192: (R)-1-(3-(5-(3-Hydroxy-1-methyl-2-oxopyrrolidin-3-yl)isoxazol-3-yl)phenyl)-4-methyl-1H-pyrazole-3-carboxamide

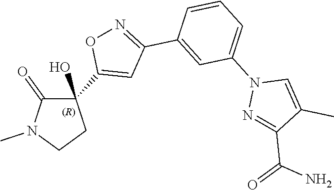

Step A: Ethyl (R)-1-(3-(5-(3-hydroxy-1-methyl-2-oxopyrrolidin-3-yl)isoxazol-3-yl)phenyl)-4-methyl-1H-pyrazole-3-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 174 mg, 0.45 mmol), ethyl 4-methyl-1H-pyrazole-3-carboxylate (50 mg, 0.32 mmol), Cu(OAc)₂ (17 mg, 0.099 mmol), pyridine (0.08 mL, 1.01 mmol), and DMF (0.65 mL) were added to a 5 mL vial, which was subsequently evacuated and refilled with Ar (×3), and stirred for 12 h at 120° C. The resulting mixture was diluted with water and extracted with ethyl acetate. The resulting organic solution was then dried over MgSO₄, filtered, and evaporated to dryness to afford a green residue (133 mg) which was used without further purification. MS (ESI): Mass calcd. for C₂₁H₂₂N₄O₅ 410.2 m/z, found 411.2 [M+H]⁺.

Step B: (R)-1-(3-(5-(3-Hydroxy-1-methyl-2-oxopyrrolidin-3-yl)isoxazol-3-yl)phenyl)-4-methyl-1H-pyrazole-3-carboxamide. A vial charged with ethyl (R)-1-(3-(5-(3-hydroxy-1-methyl-2-oxopyrrolidin-3-yl)isoxazol-3-yl)phenyl)-4-methyl-1H-pyrazole-3-carboxylate (100 mg, 0.25 mmol) in 0.72 mL of methanolic ammonia (7 M) was heated at 110° C. for 24 hours. The resulting solution was filtered through a syringe filter and evaporated to dryness. The resulting residue was purified by preparative HPLC (Waters XBridge BEH C18, 5 m, 30×150 mm using a 10 to 50% gradient of ACN/20 mM NH₄OH in H₂O over 15 min. Detection, UV at 25° C.=220-254 nM) to yield an orange residue (5 mg, 5%). MS (ESI): Mass calcd. for C₁₉H₁₉N₅O₄ 381.1 m/z, found 382.2 [M+H]⁺. ¹H NMR (500 MHz, CD₃OD) δ 8.33 (t, J=1.9 Hz, 1H), 8.19 (q, J=0.9 Hz, 1H), 7.94 (ddd, J=8.2, 2.3, 1.0 Hz, 1H), 7.82 (ddd, J=7.8, 1.6, 1.0 Hz, 1H), 7.61 (t, J=7.9 Hz, 1H), 7.00 (s, 1H), 3.61-3.54 (m, 2H), 2.96 (s, 3H), 2.74 (ddd, J=13.5, 7.0, 4.8 Hz, 1H), 2.40 (ddd, J=13.6, 8.1, 6.6 Hz, 1H), 2.37 (d, J=0.9 Hz, 3H).

### Example 193: (R)-1-(3-(5-(3-Hydroxy-1-methyl-2-oxopyrrolidin-3-yl)isoxazol-3-yl)phenyl)-1H-pyrazole-3-carboxamide

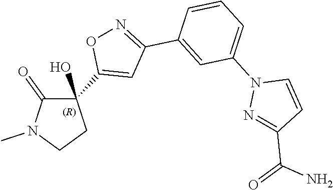

Step A: Methyl (R)-1-(3-(5-(3-hydroxy-1-methyl-2-oxopyrrolidin-3-yl)isoxazol-3-yl)phenyl)-1H-pyrazole-3-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 212 mg, 0.55 mmol), methyl 1H-pyrazole-3-carboxylate (50 mg, 0.40 mmol), Cu(OAc)₂ (22 mg, 0.12 mmol), pyridine (0.1 mL, 1.23 mmol), and DMF (0.79 mL) were added to a vial, which was subsequently evacuated and refilled with Ar (×3), and heated for 12 h at 120° C. The resulting mixture was diluted with water and extracted with ethyl acetate. The resulting organic solution was then dried over MgSO₄, filtered, and evaporated to dryness to afford a green residue (151 mg) which was used without further purification. MS (ESI): Mass calcd. for C₁₉H₁₈N₄O₅ 382.1 m/z, found 383.1 [M+H]⁺.

Step B: (R)-1-(3-(5-(3-hydroxy-1-methyl-2-oxopyrrolidin-3-yl)isoxazol-3-yl)phenyl)-1H-pyrazole-3-carboxamide. Methyl (R)-1-(3-(5-(3-hydroxy-1-methyl-2-oxopyrrolidin-3-yl)isoxazol-3-yl)phenyl)-1H-pyrazole-3-carboxylate is dissolved in 0.75 mL of methanolic ammonia (7 M) and was heated at 110° C. for 24 hours. The resulting solution was filtered through a syringe filter and evaporated to dryness. The resulting residue was purified by preparative HPLC (Waters XBridge BEH C18, 5 μm, 30×150 mm using a 10 to 50% gradient of ACN/20 mM NH₄OH in H₂O over 15 min. Detection, UV at 25° C.=220-254 nM) to yield an orange residue (5 mg, 5%). MS (ESI): Mass calcd. for C₁₈H₁₇N₅O₄ 367.1 m/z, found 368.1 [M+H]⁺. ¹H NMR (400 MHz, CD₃OD) δ 8.44 (d, J=2.6 Hz, 1H), 8.40 (t, J=1.9 Hz, 1H), 8.01 (ddd, J=8.2, 2.3, 1.0 Hz, 1H), 7.88 (ddd, J=7.8, 1.6, 1.0 Hz, 1H), 7.66 (t, J=8.0 Hz, 1H), 7.03 (s, 1H), 7.00 (d, J=2.6 Hz, 1H), 3.63-3.57 (m, 2H), 2.99 (s, 3H), 2.76 (ddd, J=13.5, 6.9, 5.0 Hz, 1H), 2.43 (ddd, J=13.4, 7.9, 6.6 Hz, 1H).

### Example 194: (R)-1-(6-(5-(3-Hydroxy-1-methyl-2-oxopyrrolidin-3-yl)isoxazol-3-yl)pyridin-2-yl)-1H-pyrazole-4-carboxamide

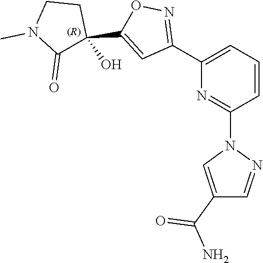

A mixture of (R)-3-(3-(6-bromopyridin-2-yl)isoxazol-5-yl)-3-hydroxy-1-methylpyrrolidin-2-one (50 mg, 0.148 mmol, Intermediate 43), 1H-pyrazole-4-carboxamide (24.6 mg, 0.222 mmol), Cs₂CO₃ (144 mg, 0.444 mmol), and DMF (1 mL, 12.915 mmol) was heated to 100° C. for 24 h. The reaction mixture was diluted with water and extracted with ethyl acetate and then 9:1 v/v DCM:IPA. The organic extracts were combined, dried with MgSO₄, filtered, and concentrated under reduced pressure.

The resulting residue was purified by reverse phase HPLC using an HPLC column, such as a Waters XBridge BEH C18, 5 μm, 30×150 mm column using an 8% to 35% gradient of CH₃CN/H₂O (pH 10 ammonium hydroxide) to afford (R)-1-(6-(5-(3-hydroxy-1-methyl-2-oxopyrrolidin-3-yl)isoxazol-3-yl)pyridin-2-yl)-1H-pyrazole-4-carboxamide (11.2 mg, 20.6%) as a white powder. MS (ESI): Mass calcd. for C₁₇H₁₆N₆O₄, 368.1; m/z found, 369.3 [M+H]⁺. ¹H NMR (400 MHz, DMSO-d₆) δ 9.28 (d, J=0.8 Hz, 1H), 8.23-8.15 (m, 2H), 8.06 (dd, J=8.2, 1.0 Hz, 1H), 8.01 (dd, J=7.6, 0.9 Hz, 1H), 7.91 (s, 1H), 7.28 (s, 1H), 7.19 (s, 1H), 6.79 (s, 1H), 3.56-3.40 (m, 2H), 2.86 (s, 3H), 2.67-2.54 (m, 1H), 2.37-2.22 (m, 1H).

### Example 195: (R)-3-Amino-1-(6-(5-(3-Hydroxy-1-methyl-2-oxopyrrolidin-3-yl)isoxazol-3-yl)pyridin-2-yl)-1H-pyrazole-4-carboxamide

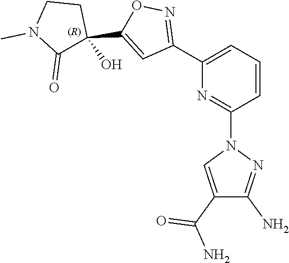

A mixture of (R)-3-(3-(6-bromopyridin-2-yl)isoxazol-5-yl)-3-hydroxy-1-methylpyrrolidin-2-one (50.0 mg, 0.148 mmol, Intermediate 43), 3-amino-1H-pyrazole-4-carboxamide (24.6 mg, 0.195 mmol), Cs₂CO₃ (144 mg, 0.444 mmol) and DMF (1 mL, 12.9 mmol) was heated at 100° C. for 24 h. The reaction mixture was diluted with water and extracted with ethyl acetate and then 9:1 v/v DCM:IPA. The organic extracts were combined, dried with MgSO₄, filtered, and concentrated under reduced pressure. The resulting residue was purified by reverse phase HPLC using an HPLC column, such as a Waters XBridge BEH C18, 5 μm, 30×150 mm column using an 8% to 35% gradient of CH₃CN/H₂O (pH 10 ammonium hydroxide) to afford (R)-3-amino-1-(6-(5-(3-hydroxy-1-methyl-2-oxopyrrolidin-3-yl)isoxazol-3-yl)pyridin-2-yl)-1H-pyrazole-4-carboxamide (7.3 mg, 13%) as a white powder. MS (ESI): Mass calcd. for C₁₇H₁₇N₇O₄, 383.1; m/z found, 384.3 [M+H]⁺. ¹H NMR (400 MHz, DMSO-d₆) δ 9.16 (s, 1H), 8.08 (t, J=7.9 Hz, 1H), 7.85 (dd, J=7.7, 0.9 Hz, 1H), 7.83-7.68 (m, 2H), 7.09 (s, 2H), 6.79 (s, 1H), 5.91 (s, 2H), 3.59-3.38 (m, 2H), 2.86 (s, 3H), 2.63-2.54 (m, 1H), 2.36-2.22 (m, 1H).

### Example 196: (R)-5-(6-(5-(3-Hydroxy-1-methyl-2-oxopyrrolidin-3-yl)isoxazol-3-yl)pyridin-2-yl)isoquinolin-1(2H)-one

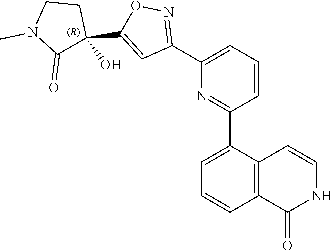

A mixture of (R)-3-(3-(6-bromopyridin-2-yl)isoxazol-5-yl)-3-hydroxy-1-methylpyrrolidin-2-one (50 mg, 0.148 mmol, Intermediate 43), 5-(4,4,5,5-tetramethyl-1,3,2-dioxaborolan-2-yl)isoquinolin-1(2H)-one (60 mg, 0.22 mmol), 1,4-dioxane (0.5 mL), K₂CO₃ (0.37 mL, 1 M in water, 0.37 mmol) and [1,1′-bis(diphenylphosphino)ferrocene]dichloropalladium(II) (10.9 mg, 0.0148 mmol) was heated at 100° C. for 2 h. The reaction mixture was diluted with water and extracted with ethyl acetate. The organic extract was dried with MgSO₄, filtered, and concentrated under reduced pressure. The resulting residue was purified by reverse phase HPLC using an HPLC column such as a Waters XSelect CSH C18, 5 μm, 30×150 mm column using a 10% to 45% gradient of MeCN/H₂O (0.16% TFA) to afford (R)-5-(6-(5-(3-hydroxy-1-methyl-2-oxopyrrolidin-3-yl)isoxazol-3-yl)pyridin-2-yl)isoquinolin-1(2H)-one (50.8 mg, 85.4%) as a yellow powder. MS (ESI): Mass calcd. for C₂₂H₁₈N₄O₄, 402.1; m/z found, 403.1 [M+H]⁺. ¹H NMR (400 MHz, DMSO-d₆) δ 11.45-11.28 (m, 1H), 8.42-8.31 (m, 1H), 8.13 (t, J=7.7 Hz, 1H), 8.10-8.05 (m, 1H), 7.89 (dd, J=7.4, 1.4 Hz, 1H), 7.77 (dd, J=7.6, 1.1 Hz, 1H), 7.62 (t, J=7.7 Hz, 1H), 7.23-7.14 (m, 1H), 6.96 (s, 1H), 6.68 (d, J=7.4 Hz, 1H), 3.51-3.38 (m, 2H), 2.83 (s, 3H), 2.64-2.54 (m, 1H), 2.31-2.21 (m, 1H).

### Example 197: (R)-8-(3-(5-(3-Hydroxy-1-methyl-2-oxopyrrolidin-3-yl)isoxazol-3-yl)phenyl)quinazolin-4(3H)-one

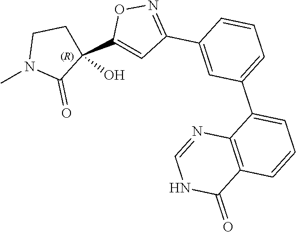

A mixture of Intermediate 4: (R)-3-Hydroxy-1-methyl-3-(3-(3-(4,4,5,5-tetramethyl-1,3,2-dioxaborolan-2-yl)phenyl)isoxazol-5-yl)pyrrolidin-2-one. (Intermediate 4, 57 mg, 0.148 mmol), 8-bromoquinazolin-4(3H)-one (33 mg, 0.15 mmol), [1,1′-bis(diphenylphosphino)ferrocene]dichloropalladium(II) (10.9 mg, 0.0148 mmol), 1,4-dioxane (0.57 mL), and K₂CO₃ (0.371 mL, 1 M in water, 0.371 mmol) was heated to 100° C. for 2 h. The reaction mixture was diluted with water and extracted with ethyl acetate. The organic extract was dried with MgSO₄, filtered and concentrated. The resulting residue was purified by reverse phase HPLC using an HPLC column, such as a Waters XBridge BEH C18, 5 μm, 30×150 mm column using a 10% to 40% gradient of CH₃CN/H₂O (pH 10 ammonium hydroxide) to afford (R)-8-(3-(5-(3-hydroxy-1-methyl-2-oxopyrrolidin-3-yl)isoxazol-3-yl)phenyl)quinazolin-4(3H)-one (16.8 mg, 28.1%) as a yellow powder. MS (ESI): Mass calcd. for C₂₂H₁₈N₄O₄, 402.1; m/z found, 403.4 [M+H]⁺. ¹H NMR (400 MHz, DMSO-d₆) δ 8.20 (dd, J=7.9, 1.6 Hz, 1H), 8.12 (s, 1H), 8.09-8.03 (m, 1H), 7.96-7.84 (m, 2H), 7.77-7.67 (m, 1H), 7.67-7.54 (m, 2H), 7.05 (s, 1H), 3.52-3.39 (m, 2H), 2.84 (s, 3H), 2.61-2.52 (m, 1H), 2.34-2.21 (m, 1H).

### Example 198: (R)-6′-(5-(3-Hydroxy-1-methyl-2-oxopyrrolidin-3-yl)isoxazol-3-yl)-[2,2′-bipyridine]-4-carboxamide

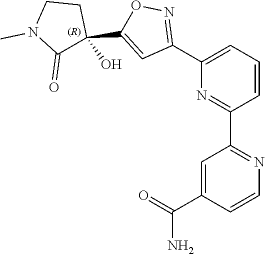

A mixture of (R)-3-(3-(6-bromopyridin-2-yl)isoxazol-5-yl)-3-hydroxy-1-methylpyrrolidin-2-one (100 mg, 0.296 mmol, Intermediate 43), 2-chloroisonicotinamide (46 mg, 0.30 mmol), tetrakis(triphenylphosphine)palladium(0) (34 mg, 0.030 mmol), hexamethylditin (97 mg, 0.30 mmol) and DMF (1 mL) was heated to 160° C. in a microwave reactor for 1 h. The reaction mixture was diluted with NaOH solution (1 M in water) and extracted with ethyl acetate. The organic extract was dried with MgSO₄, filtered, and concentrated under reduced pressure. The resulting residue was purified by reverse phase HPLC using an HPLC column, such as a Waters XBridge BEH C18, 5 μm, 30×150 mm column using an 8% to 35% gradient of CH₃CN/H₂O (pH 10 ammonium hydroxide) to afford (R)-6′-(5-(3-hydroxy-1-methyl-2-oxopyrrolidin-3-yl)isoxazol-3-yl)-[2,2′-bipyridine]-4-carboxamide (12.7 mg, 11.3%) as a white powder. MS (ESI): Mass calcd. for C₁₉H₁₇N₅O₄, 379.1; m/z found, 380.0 [M+H]⁺. ¹H NMR (400 MHz, DMSO-d₆) δ 8.91-8.82 (m, 2H), 8.53 (dd, J=7.6, 1.3 Hz, 1H), 8.47 (s, 1H), 8.19-8.02 (m, 2H), 7.93-7.85 (m, 1H), 7.83 (s, 1H), 7.24 (s, 1H), 6.83 (s, 1H), 3.56-3.43 (m, 2H), 2.86 (s, 3H), 2.66-2.57 (m, 1H), 2.36-2.25 (m, 1H).

### Example 199 and Example 200: (R)-3-(3-((R)-1-(4-Amino-8-methylpyrido[3,2-d]pyrimidin-6-yl)piperidin-3-yl)isoxazol-5-yl)-3-hydroxy-1-methylpyrrolidin-2-one and (R)-3-(3-((S)-1-(4-Amino-8-methylpyrido[3,2-d]pyrimidin-6-yl)piperidin-3-yl)isoxazol-5-yl)-3-hydroxy-1-methylpyrrolidin-2-one

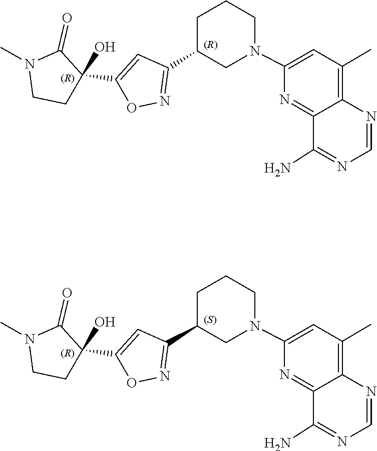

Step A: (3R)-3-Hydroxy-1-methyl-3-(3-(piperidin-3-yl)isoxazol-5-yl)pyrrolidin-2-one (2000 mg, Intermediate 63), 6-chloro-8-methylpyrido[3,2-d]pyrimidin-4-amine (Example 26 Step C, 293 mg, 1.51 mmol), TEA (525 μL, 3.77 mmol), and DMA (10 mL) were added to a 20 mL microwave tube. The resultant mixture was heated at 140° C. via microwave irradiation for 3 h. After this time, the reaction vessel was removed from the microwave heating device and allowed to gradually cool to room temperature. The mixture was concentrated to dryness in vacuo. The residue was subjected to silica gel chromatography (0-10% MeOH/DCM), and then purified by HPLC (Phenomenex Gemini-NX C18 column, 3 μm, 75×30 mm, 14-44% (v/v) CH₃CN/H₂O with 0.05% NH₃H₂O+10 mM NH₄HCO₃) to afford, after lyophilization, (R)-3-(3-((R,S)-1-(4-amino-8-methylpyrido[3,2-d]pyrimidin-6-yl)piperidin-3-yl)isoxazol-5-yl)-3-hydroxy-1-methylpyrrolidin-2-one as a white solid (70 mg, 9%). MS (ESI): Mass calcd. for C₂₁H₂₅N₇O₃ 423.20, found 424.2 [M+H]⁺.

Step B: The (R) and (S) diastereomers of the racemic mixture of (R)-3-(3-((R,S)-1-(4-amino-8-methylpyrido[3,2-d]pyrimidin-6-yl)piperidin-3-yl)isoxazol-5-yl)-3-hydroxy-1-methylpyrrolidin-2-one (70 mg, 0.165 mmol) were separated by SFC (AD column, 10 μm, 250×30 mm; 45% IPA with 0.1% NH₃H₂O/supercritical CO₂) to give two products. The first eluting product was designated as diastereomer 1 of (R)-3-(3-(1-(4-amino-8-methylpyrido[3,2-d]pyrimidin-6-yl)piperidin-3-yl)isoxazol-5-yl)-3-hydroxy-1-methylpyrrolidin-2-one (Example 199), and was obtained as a white solid (16.4 mg, 23% yield, RT of SFC=8.349 min). MS (ESI): Mass calcd. for C₂₁H₂₅N₇O₃ 423.20 m/z, found 424.1 [M+H]⁺. ¹H NMR (400 MHz, DMSO-d₆) δ 8.18 (s, 1H), 7.43 (s, 1H), 7.37-7.18 (m, 2H), 6.63 (s, 1H), 6.58 (s, 1H), 4.66-4.56 (m, 1H), 4.52-4.43 (m, 1H), 3.37-3.33 (m, 2H), 3.20-3.14 (m, 1H), 3.10-3.01 (m, 1H), 2.97-2.88 (m, 1H), 2.81 (s, 3H), 2.49-2.42 (m, 4H), 2.26-2.17 (m, 1H), 2.10-2.00 (m, 1H), 1.83-1.69 (m, 2H), 1.65-1.55 (m, 1H). The second eluting product was designated as diastereomer 2 of (R)-3-(3-(1-(4-amino-8-methylpyrido[3,2-d]pyrimidin-6-yl)piperidin-3-yl)isoxazol-5-yl)-3-hydroxy-1-methylpyrrolidin-2-one (Example 200), and was obtained as a white solid (11.5 mg, 15.5% yield, RT of SFC=9.872 min). MS (ESI): Mass calcd. for C₂₁H₂₅N₇O₃ 423.20 m/z, found 424.1 [M+H]⁺. ¹H NMR (400 MHz, DMSO-d₆) δ 8.18 (s, 1H), 7.42 (s, 1H), 7.38-7.17 (m, 2H), 6.63 (s, 1H), 6.57 (s, 1H), 4.65-4.55 (m, 1H), 4.52-4.41 (m, 1H), 3.37-3.33 (m, 2H), 3.20-3.15 (m, 1H), 3.10-3.01 (m, 1H), 2.98-2.89 (m, 1H), 2.80 (s, 3H), 2.49-2.43 (m, 4H), 2.26-2.16 (m, 1H), 2.11-2.01 (m, 1H), 1.83-1.72 (m, 2H), 1.66-1.54 (m, 1H).

### Example 201: (3R)-3-(3-(1-(4-Aminopyrido[3,2-d]pyrimidin-6-yl)piperidin-3-yl)isoxazol-5-yl)-3-hydroxy-1-methylpyrrolidin-2-one

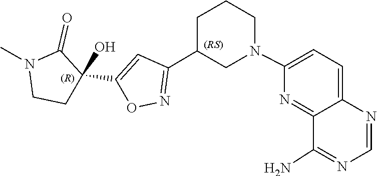

(3R)-3-Hydroxy-1-methyl-3-(3-(piperidin-3-yl)isoxazol-5-yl)pyrrolidin-2-one (Intermediate 63, 1.90 g, 20 wt. %, 1.43 mmol), 6-chloropyrido[3,2-d]pyrimidin-4-amine (259 mg, 1.43 mmol, Intermediate 59), TEA (500 μL, 3.59 mmol), and DMA (10 mL) were added into a 20 mL microwave tube. The resultant mixture was stirred while heating at 140° C. via microwave irradiation for 3 h. After this time, the reaction vessel was removed from the microwave heating device and allowed to gradually cool to room temperature. The mixture was concentrated to dryness in vacuo. The residue was subjected to silica gel chromatography (0-10% MeOH/DCM), and then purified by HPLC (Xtimate C18 column, 5 μm, 150×40 mm, 17-44% (v/v) CH₃CN/H₂O with 0.05% NH₃H₂O) to afford, after lyophilization, (3R)-3-(3-(1-(4-aminopyrido[3,2-d]pyrimidin-6-yl)piperidin-3-yl)isoxazol-5-yl)-3-hydroxy-1-methylpyrrolidin-2-one as a white solid (150 mg, 26%). MS (ESI): Mass calcd. for C₂₀H₂₃N₇O₃ 409.19 m/z found 410.1 [M+1]⁺.

### Example 202 and Example 203: (R)-3-(3-((R)-1-(4-aminopyrido[3,2-d]pyrimidin-6-yl)piperidin-3-yl)isoxazol-5-yl)-3-hydroxy-1-methylpyrrolidin-2-one and (R)-3-(3-((S)-1-(4-Aminopyrido[3,2-d]pyrimidin-6-yl)piperidin-3-yl)isoxazol-5-yl)-3-hydroxy-1-methylpyrrolidin-2-one

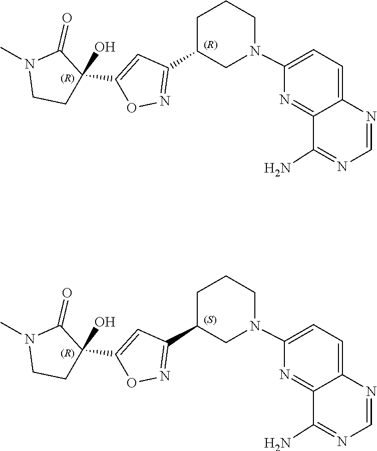

Step A: tert-Butyl (tert-butoxycarbonyl)(6-((R,S)-3-(5-((R)-3-hydroxy-1-methyl-2-oxopyrrolidin-3-yl)isoxazol-3-yl)piperidin-1-yl)pyrido[3,2-d]pyrimidin-4-yl)carbamate. Boc₂O (139 mg, 0.637 mmol) was added to a solution consisting of (3R)-3-(3-(1-(4-aminopyrido[3,2-d]pyrimidin-6-yl)piperidin-3-yl)isoxazol-5-yl)-3-hydroxy-1-methylpyrrolidin-2-one (Example 201, 130 mg, 0.318 mmol), DMAP (116 mg, 0.950 mmol), and DCM (20 mL). The resultant mixture was stirred at room temperature for 16 h. After this time, the reaction was quenched with water (25 mL) and extracted with DCM (3×25 mL). The organic layers were combined, dried over anhydrous Na₂SO₄, filtered, and concentrated to dryness in vacuo to give tert-butyl (tert-butoxycarbonyl)(6-((R,S)-3-(5-((R)-3-hydroxy-1-methyl-2-oxopyrrolidin-3-yl)isoxazol-3-yl)piperidin-1-yl)pyrido[3,2-d]pyrimidin-4-yl)carbamate (180 mg) as a white solid. MS (ESI): Mass calcd. for C₃₀H₃₉N₇O₇ 609.29 m/z found 610.2 [M+1]⁺.

tert-Butyl (tert-butoxycarbonyl)(6-((R)-3-(5-((R)-3-hydroxy-1-methyl-2-oxopyrrolidin-3-yl)isoxazol-3-yl)piperidin-1-yl)pyrido[3,2-d]pyrimidin-4-yl)carbamate and tert-butyl (tert-butoxycarbonyl)(6-((S)-3-(5-((R)-3-hydroxy-1-methyl-2-oxopyrrolidin-3-yl)isoxazol-3-yl)piperidin-1-yl)pyrido[3,2-d]pyrimidin-4-yl)carbamate. The (R) and (S) diastereomers of the racemic mixture of tert-butyl (tert-butoxycarbonyl)(6-((R,S)-3-(5-((R)-3-hydroxy-1-methyl-2-oxopyrrolidin-3-yl)isoxazol-3-yl)piperidin-1-yl)pyrido[3,2-d]pyrimidin-4-yl)carbamate (180 mg, 0.295 mmol) were separated by SFC (IG column, 10 μm, 250 mm×30 mm; 50% (v/v) EtOH (containing 0.1% of 25% aq. NH₃)/CO₂) to give two products. The first eluting product was designated as diastereomer 1 of tert-butyl (tert-butoxycarbonyl)(6-(3-(5-((R)-3-hydroxy-1-methyl-2-oxopyrrolidin-3-yl)isoxazol-3-yl)piperidin-1-yl)pyrido[3,2-d]pyrimidin-4-yl)carbamate and was obtained as a white solid (45 mg, 25%). MS (ESI): Mass calcd. for C₃₀H₃₉N₇O₇ 609.29 m/z found 610.2 [M+1]⁺. The second eluting product was designated as diastereomer 2 of tert-butyl (tert-butoxycarbonyl)(6-(3-(5-((R)-3-hydroxy-1-methyl-2-oxopyrrolidin-3-yl)isoxazol-3-yl)piperidin-1-yl)pyrido[3,2-d]pyrimidin-4-yl)carbamate.

Step C: (R)-3-(3-((R)-1-(4-aminopyrido[3,2-d]pyrimidin-6-yl)piperidin-3-yl)isoxazol-5-yl)-3-hydroxy-1-methylpyrrolidin-2-one and (R)-3-(3-((S)-1-(4-aminopyrido[3,2-d]pyrimidin-6-yl)piperidin-3-yl)isoxazol-5-yl)-3-hydroxy-1-methylpyrrolidin-2-one. TMSCl (84 μL, 0.66 mmol) was added dropwise to a 0° C. mixture consisting of diastereomer 1 of tert-butyl (tert-butoxycarbonyl)(6-(3-(5-((R)-3-hydroxy-1-methyl-2-oxopyrrolidin-3-yl)isoxazol-3-yl)piperidin-1-yl)pyrido[3,2-d]pyrimidin-4-yl)carbamate (40 mg, 0.066 mmol), NaI (98.3 mg, 0.656 mmol), and CH₃CN (5 mL). The resultant mixture was stirred at room temperature for 16 h, quenched with NH₄OH (0.5 mL, 28 wt. % in H₂O), and concentrated to dryness in vacuo. The residue was subjected to HPLC (Boston Prime C18 column, 5 μm, 150×30 mm; 20-50% (v/v) CH₃CN/H₂O with 0.05% NH₃H₂O+10 mM NH₄HCO₃) to give, after lyophilization, diastereomer 1 of (R)-3-(3-(1-(4-aminopyrido[3,2-d]pyrimidin-6-yl)piperidin-3-yl)isoxazol-5-yl)-3-hydroxy-1-methylpyrrolidin-2-one (Example 202) as a white solid (10.2 mg, 38%). MS (ESI): Mass calcd. for C₂₀H₂₃N₇O₃ 409.19 m/z found 410.2 [M+1]⁺. ¹H NMR (400 MHz, DMSO-d₆) δ 8.16 (s, 1H), 7.77 (d, J=9.3 Hz, 1H), 7.53 (d, J=9.3 Hz, 1H), 7.49-7.22 (m, 2H), 6.78-6.40 (m, 2H), 4.68-4.58 (m, 1H), 4.49-4.41 (m, 1H), 3.47-3.41 (m, 2H), 3.24-3.19 (m, 1H), 3.14-3.06 (m, 1H), 2.99-2.91 (m, 1H), 2.81 (s, 3H), 2.46-2.41 (m, 1H), 2.25-2.16 (m, 1H), 2.08-2.01 (m, 1H), 1.83-1.74 (m, 2H), 1.67-1.56 (m, 1H).

Diastereomer 2 of (R)-3-(3-(1-(4-aminopyrido[3,2-d]pyrimidin-6-yl)piperidin-3-yl)isoxazol-5-yl)-3-hydroxy-1-methylpyrrolidin-2-one (Example 203; 10.5 mg, 34%) was made analogously to Example 202, using diastereomer 2 of tert-butyl (tert-butoxycarbonyl)(6-(3-(5-((R)-3-hydroxy-1-methyl-2-oxopyrrolidin-3-yl)isoxazol-3-yl)piperidin-1-yl)pyrido[3,2-d]pyrimidin-4-yl)carbamate in place of diastereomer 1 of tert-butyl (tert-butoxycarbonyl)(6-(3-(5-((R)-3-hydroxy-1-methyl-2-oxopyrrolidin-3-yl)isoxazol-3-yl)piperidin-1-yl)pyrido[3,2-d]pyrimidin-4-yl)carbamate. MS (ESI): Mass calcd. for C₂₀H₂₃N₇O₃ 409.19 m/z found 410.1 [M+1]⁺. ¹H NMR (400 MHz, DMSO-d₆) δ 8.16 (s, 1H), 7.77 (d, J=9.4 Hz, 1H), 7.53 (d, J=9.4 Hz, 1H), 7.47-7.19 (m, 2H), 6.65-6.54 (m, 2H), 4.67-4.57 (m, 1H), 4.51-4.41 (m, 1H), 3.47-3.39 (m, 2H), 3.25-3.19 (m, 1H), 3.16-3.07 (m, 1H), 2.99-2.91 (m, 1H), 2.80 (s, 3H), 2.47-2.42 (m, 1H), 2.25-2.16 (m, 1H), 2.08-2.01 (m, 1H), 1.83-1.71 (m, 2H), 1.66-1.55 (m, 1H).

### Example 204: (R)-2-(3-(5-(3-Hydroxy-1-methyl-2-oxopyrrolidin-3-yl)isoxazol-3-yl)phenyl)thiazole-4-carboxamide

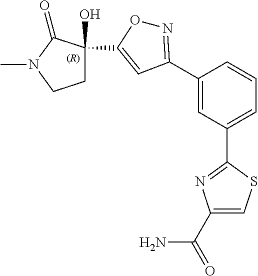

A mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 140 mg, 0.36 mmol), 2-bromothiazole-4-carboxamide (80 mg, 0.39 mmol), K₃PO₄ (149 mg, 1.09 mmol), 1,4-dioxane (5 mL), and water (0.5 mL) was evacuated and refilled with N₂ 3 times, treated with Pd(dtbpf)Cl₂ (24 mg, 0.036 mmol), and heated at 100° C. for 2 h under N₂. The reaction vessel was removed from the oil bath and allowed to gradually cool to rt. The mixture was filtered through a pad of diatomaceous earth and the pad was washed with EtOAc (15 mL). The filtrate was concentrated in vacuo. The residue was subjected to HPLC (Boston Green ODS column, 5 μm, 150×30 mm, 30-60% (v/v) CH₃CN/H₂O with 0.225% FA) to give, after lyophilization, (R)-2-(3-(5-(3-hydroxy-1-methyl-2-oxopyrrolidin-3-yl)isoxazol-3-yl)phenyl)thiazole-4-carboxamide as a white solid (33.3 mg, 24%). MS (ESI): Mass calcd. for C₁₈H₁₆N₄O₄S 384.09 m/z, found 385.0 [M+H]⁺. ¹H NMR (400 MHz, DMSO-d₆) δ 8.52 (s, 1H), 8.35 (s, 1H), 8.16 (d, J=8.0 Hz, 1H), 8.10-8.01 (m, 2H), 7.77-7.66 (m, 2H), 7.17 (s, 1H), 6.79 (s, 1H), 3.53-3.47 (m, 2H), 2.85 (s, 3H), 2.63-2.55 (m, 1H), 2.34-2.25 (m, 1H). ¹H NMR (400 MHz, CD₃OD) δ 8.55 (s, 1H), 8.29 (s, 1H), 8.15 (d, J=8.0 Hz, 1H), 8.00 (d, J=8.0 Hz, 1H), 7.70-7.61 (m, 1H), 7.04 (s, 1H), 3.64-3.56 (m, 2H), 2.99 (s, 3H), 2.81-2.72 (m, 1H), 2.48-2.38 (m, 1H).

### Example 205: (R)-4-(3-(5-(3-Hydroxy-1-methyl-2-oxopyrrolidin-3-yl)isoxazol-3-yl)phenyl)thiazole-2-carboxamide

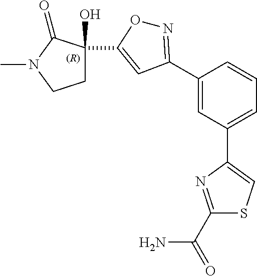

Step A: 4-Bromothiazole-2-carbonyl chloride. To a mixture consisting of 4-bromothiazole-2-carboxylic acid (500 mg, 2.4 mmol) and SOCl₂ (5 mL) was added anhydrous DMF (50 mg, 0.68 mmol) in one portion. The resultant mixture was heated at 80° C. for 2 h to afford a clear light yellow solution. The reaction vessel was removed from the oil bath and allowed to gradually cool to RT. The reaction mixture was concentrated in vacuo to afford 4-bromothiazole-2-carbonyl chloride as a light yellow gum (600 mg), which was used for the next step directly.

Step B: 4-Bromothiazole-2-carboxamide. To a solution consisting of 4-bromothiazole-2-carbonyl chloride (600 mg, 2.65 mmol), and anhydrous CH₂Cl₂ (20 mL) was added NH₃ (0.95 mL, 6.6 mmol, 7 M in MeOH) in portions at 0° C. over 5 min, and stirred for 1 h with gradual warming to room temperature. After this time, the solution was concentrated to dryness in vacuo. The residue was subjected to silica gel chromatography (0-100% EtOAc/petroleum ether) to afford 4-bromothiazole-2-carboxamide (180 mg, 33%) as a yellow solid. MS (ESI): Mass calcd. for C₄H₃BrN₂OS 205.91 m/z, found 206.9 [M+H]⁺.

Step C: (R)-4-(3-(5-(3-Hydroxy-1-methyl-2-oxopyrrolidin-3-yl)isoxazol-3-yl)phenyl)thiazole-2-carboxamide. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 140 mg, 0.36 mmol), 4-bromothiazole-2-carboxamide (82 mg, 0.40 mmol), K₃PO₄ (149 mg, 1.09 mmol), 1,4-dioxane (5 mL), and water (0.5 mL) was evacuated and refilled with N₂ for 3 times, treated with Pd(dtbpf)Cl₂ (24 mg, 0.036 mmol), and heated at 100° C. for 20 h under N₂. The reaction vessel was removed from the oil bath and allowed to gradually cool to rt. The reaction mixture was filtered through a pad of diatomaceous earth and the pad washed with EtOAc (15 mL). The filtrate was concentrated to dryness in vacuo. The residue was subjected to HPLC (Boston Green ODS column, 5 μm, 150×30 mm; 25-55% CH₃CN/H₂O with 0.225% FA) to give, after lyophilization (R)-4-(3-(5-(3-hydroxy-1-methyl-2-oxopyrrolidin-3-yl)isoxazol-3-yl)phenyl)thiazole-2-carboxamide as a white solid (65.9 mg, 47%). MS (ESI): Mass calcd. for C₁₈H₁₆N₄O₄S 384.09 m/z, found 385.1 [M+H]⁺. ¹H NMR (400 MHz, DMSO-d₆) δ 8.57 (s, 1H), 8.54 (s, 1H), 8.38 (s, 1H), 8.20 (d, J=8.0 Hz, 1H), 7.97 (s, 1H), 7.90 (d, J=8.0 Hz, 1H), 7.66-7.59 (m, 1H), 7.13 (s, 1H), 6.79 (s, 1H), 3.58-3.50 (m, 2H), 2.85 (s, 3H), 2.64-2.54 (m, 1H), 2.34-2.24 (m, 1H). ¹H NMR (400 MHz, CD₃OD) δ 8.54 (s, 1H), 8.26 (s, 1H), 8.15 (d, J=8.0 Hz, 1H), 7.88 (d, J=8.0 Hz, 1H), 7.64-7.56 (m, 1H), 7.03 (s, 1H), 3.64-3.56 (m, 2H), 2.99 (s, 3H), 2.82-2.71 (m, 1H), 2.48-2.38 (m, 1H).

### Example 206: (R)-5-Amino-2-(3-(5-(3-hydroxy-1-methyl-2-oxopyrrolidin-3-yl)isoxazol-3-yl)phenyl)thiazole-4-carboxamide

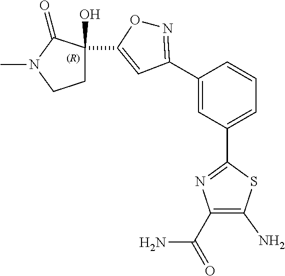

Step A: 5-Amino-2-iodothiazole-4-carboxamide. To a solution consisting of 5-aminothiazole-4-carboxamide (1.00 g, 6.99 mmol), and DMF (15 mL) was added NIS (2.36 g, 10.5 mmol) in one portion at room temperature. After addition, the solution was stirred for 2 h at room temperature. The solution was diluted with EtOAc (150 mL) and washed with water (20 mL×3) and brine (20 mL×3), dried over anhydrous Na₂SO₄, filtered, and concentrated in vacuo. The residue was subjected to silica gel chromatography (0-50% EtOAc/petroleum ether) to afford 5-amino-2-iodothiazole-4-carboxamide as a pale yellow solid (700 mg, 37%). MS (ESI): Mass calcd. for C₄H₄₁N₃OS 268.91 m/z, found 270.0 [M+H]⁺.

Step B: (R)-5-Amino-2-(3-(5-(3-hydroxy-1-methyl-2-oxopyrrolidin-3-yl)isoxazol-3-yl)phenyl)thiazole-4-carboxamide. A mixture of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 400 mg, 1.04 mmol), 5-amino-2-iodothiazole-4-carboxamide (560 mg, 2.08 mmol), Pd(dtbpf)Cl₂ (68 mg, 0.104 mmol), and 1,4-dioxane (15 mL) was sparged with N₂ for 5 min, treated with KF (2.6 mL, 2.6 mmol, 1 M in H₂O), and heated at 70° C. for 20 h under N₂ atmosphere. The reaction vessel was removed from the oil bath and allowed to gradually cool to rt. The mixture was filtered through a pad of diatomaceous earth, and the pad washed with EtOAc (25 mL). The filtrate was concentrated in vacuo. The residue was subjected to HPLC with a Boston Green ODS column, 5 μm, 150×30 mm; 20-50% CH₃CN/H₂O with 0.225% FA) to give, after lyophilization, (R)-5-amino-2-(3-(5-(3-hydroxy-1-methyl-2-oxopyrrolidin-3-yl)isoxazol-3-yl)phenyl)thiazole-4-carboxamide as an off-white solid (91.8 mg, 22%). MS (ESI): Mass calcd. for C₁₈H₁₇N₅O₄S 399.10 m/z, found 400.1 [M+H]⁺. ¹H NMR (400 MHz, DMSO-d₆) δ 8.27 (s, 1H), 7.87 (d, J=8.0 Hz, 2H), 7.63-7.54 (m, 1H), 7.44 (s, 3H), 7.14 (br s, 1H), 7.12 (s, 1H), 6.75 (br s, 1H), 3.53-3.43 (m, 2H), 2.85 (s, 3H), 2.62-2.54 (m, 1H), 2.35-2.23 (m, 1H). ¹H NMR (400 MHz, CD₃OD) δ 8.29 (s, 1H), 7.91-7.80 (m, 2H), 7.59-7.51 (m, 1H), 6.99 (s, 1H), 3.63-3.56 (m, 2H), 2.99 (s, 3H), 2.80-2.72 (m, 1H), 2.47-2.37 (m, 1H).

### Example 207: (R)-5-(3-(5-(3-Hydroxy-1-methyl-2-oxopyrrolidin-3-yl)isoxazol-3-yl)phenyl)furan-2-carboxamide

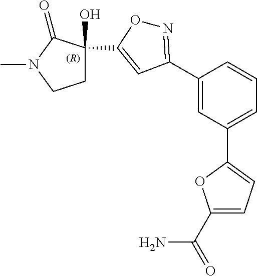

A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 140 mg, 0.36 mmol), 5-bromo-2-furamide (75 mg, 0.40 mmol), K₃PO₄ (149 mg, 1.09 mmol), 1,4-dioxane (5 mL), and water (0.5 mL) was sparged with N₂ for 5 min, treated with Pd(dtbpf)Cl₂ (24 mg, 0.036 mmol), and heated at 100° C. for 20 h. The reaction vessel was removed from the oil bath and allowed to gradually cool to rt. The reaction mixture was then filtered through a pad of diatomaceous earth, and the pad washed with EtOAc (15 mL). The filtrate was concentrated to dryness in vacuo. The residue was subjected to HPLC (Boston Green ODS column 5 μm, 150×30 mm; 25-55% (v/v) CH₃CN₂O with 0.225% FA) to give, after lyophilization, (R)-5-(3-(5-(3-hydroxy-1-methyl-2-oxopyrrolidin-3-yl)isoxazol-3-yl)phenyl)furan-2-carboxamide as a white solid (61.6 mg, 46%). MS (ESI): Mass calcd. for C₁₉H₁₇N₃O₅ 367.12 m/z, found 368.0 [M+H]⁺. ¹H NMR (400 MHz, DMSO-d₆) δ 8.36 (s, 1H), 8.09 (s, 1H), 8.04 (d, J=7.6 Hz, 1H), 7.88 (d, J=7.6 Hz, 1H), 7.67-7.58 (m, 1H), 7.54 (s, 1H), 7.26 (d, J=3.6 Hz, 1H), 7.21 (d, J=3.6 Hz, 1H), 7.15 (s, 1H), 6.79 (s, 1H), 3.54-3.48 (m, 1H), 2.85 (s, 3H), 2.63-2.54 (m, 1H), 2.37-2.24 (m, 1H). ¹H NMR (400 MHz, CD₃OD) δ 8.36 (s, 1H), 7.98 (d, J=8.0 Hz, 1H), 7.86 (d, J=8.0 Hz, 1H), 7.63-7.54 (m, 1H), 7.26 (d, J=3.6 Hz, 1H), 7.06 (d, J=3.6 Hz, 1H), 7.04 (s, 1H), 3.65-3.55 (m, 2H), 2.99 (s, 3H), 2.83-2.71 (m, 1H), 2.48-2.37 (m, 1H).

### Example 208: (R)-5-Cyclopropyl-2-(3-(5-(3-hydroxy-1-methyl-2-oxopyrrolidin-3-yl)isoxazol-3-yl)phenyl)thiazole-4-carboxamide

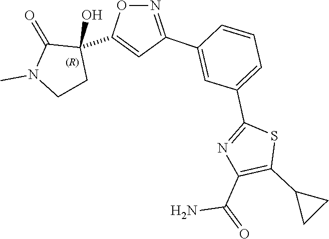

Step A: Ethyl 5-cyclopropylthiazole-4-carboxylate. Ethyl 5-bromothiazole-4-carboxylate (2.00 g, 8.47 mmol), cyclopropylboronic acid (1.60 g, 18.6 mmol), K₃PO₄ (5.40 g, 25.4 mmol), and Pd(dppf)Cl₂ (620 mg, 0.847 mmol) were added to a mixture of toluene (60 mL) and H₂O (3 mL). The resultant mixture was sparged with N₂ for 5 times and heated at 110° C. for 5 h. The reaction vessel was removed from the oil bath and allowed to gradually cool to RT. The mixture was filtered, and the filtrate concentrated to dryness in vacuo. The residue was subjected to silica gel chromatography (0-100% EtOAc/petroleum ether) to afford ethyl 5-cyclopropylthiazole-4-carboxylate as a yellow oil (1.44 g, 82.5%). MS (ESI): Mass calcd. for C₉H₁₁NO₂S 197.05 m/z, found 197.9 [M+1]⁺.

Step B: Ethyl 2-bromo-5-cyclopropylthiazole-4-carboxylate. NBS (1.95 g, 11.0 mmol) was added to a solution consisting of ethyl 5-cyclopropylthiazole-4-carboxylate (1.44 g, 7.30 mmol) in ACN (20 mL). The resulting mixture was heated at 70° C. for 18 h. The reaction vessel was removed from the oil bath and allowed to gradually cool to rt. The mixture was then quenched with sat. aqueous NaHSO₃ (40 mL) and extracted with EtOAc (40 mL×2). The combined organic extracts were dried over anhydrous Na₂SO₄, filtered, and concentrated to afford the product, which was subjected to silica gel chromatography (0-50% EtOAc/petroleum ether) to give ethyl 2-bromo-5-cyclopropylthiazole-4-carboxylate as a yellow oil (1.00 g, 40.6%). MS (ESI): Mass calcd. for C₉H₁₀BrNO₂S 274.96 m/z, found 275.6 [M+1]⁺.

Step C: 2-Bromo-5-cyclopropylthiazole-4-carboxylic acid. Lithium hydroxide monohydrate (760 mg, 18.1 mmol) was added to a solution of ethyl 2-bromo-5-cyclopropylthiazole-4-carboxylate (1.0 g, 3.62 mmol) in THF (23 mL), water (4.6 mL), and methanol (2.3 mL). The resulting mixture was stirred for 2 h at rt. After this time, the reaction mixture was acidified to pH=2 with 1.0 M aqueous HCl and extracted with EtOAc (30 mL×2). The combined organic extracts were washed with brine, dried over anhydrous Na₂SO₄, filtered, and concentrated to dryness in vacuo to give 2-bromo-5-cyclopropylthiazole-4-carboxylic acid (560 mg, 60.9%) as a pale-yellow solid. The product was used without further purification. MS (ESI): Mass calcd. for C₇H₆BrNO₂S 246.93 m/z, found 247.6 [M+1]⁺.

Step D: 2-Bromo-5-cyclopropylthiazole-4-carboxamide. To a mixture consisting of 2-bromo-5-cyclopropylthiazole-4-carboxylic acid (560 mg, 2.26 mmol) and Et₃N (0.94 mL, 6.77 mmol) in DMF (10 mL) was added HATU (1.29 g, 3.39 mmol) and NH₄Cl (1.21 g, 22.6 mmol). The resulting mixture was stirred at room temperature for 12 h. After this time, the mixture was diluted with EtOAc (30 mL) and washed with H₂O (10×3 mL). The combined organic extracts were dried over anhydrous Na₂SO₄, filtered, and concentrated to dryness in vacuo. The residue was subjected to silica gel chromatography (0-100% EtOAc/petroleum ether) to afford 2-bromo-5-cyclopropylthiazole-4-carboxamide (410 mg, 71.4%) as a white solid. MS (ESI): Mass calcd. for C₇H₇BrN₂OS 245.95 m/z, found 247.9 [M+1]⁺.

Step E: (R)-5-Cyclopropyl-2-(3-(5-(3-hydroxy-1-methyl-2-oxopyrrolidin-3-yl)isoxazol-3-yl)phenyl)thiazole-4-carboxamide. A mixture consisting of 2-bromo-5-cyclopropylthiazole-4-carboxamide (178 mg, 0.721 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 230 mg, 0.599 mmol), K₃PO₄ (318 mg, 1.50 mmol), 1,4-dioxane (4 mL), and H₂O (1 mL) was sparged with N₂ for 5 min, treated with Pd(dppf)Cl₂ (43.8 mg, 0.06 mmol), and heated at 100° C. for 3 h. After this time, the reaction vessel was removed from the oil bath and allowed to gradually cool to RT. The mixture was concentrated to dryness in vacuo. The residue was subjected to HPLC (Phenomenex Gemini-NX C18 column, 3 m, 75×30 mm; 29-59% (v/v) CH₃CN/H₂O with 0.05% NH₃H₂O+10 mM NH₄HCO₃) to give, after lyophilization, the title compound as a white solid (145.8 mg, 56%). MS (ESI): Mass calcd. for C₂₁H₂₀N₄O₄S 424.12 m/z, found 425.1 [M+H]⁺. ¹H NMR (400 MHz, DMSO-d₆) δ 8.41-8.37 (m, 1H), 8.03 (d, J=7.6 Hz, 1H), 7.98-7.92 (m, 2H), 7.63-7.56 (m, 2H), 7.14 (s, 1H), 6.73 (s, 1H), 3.50-3.35 (m, 3H), 2.81 (s, 3H), 2.58-2.50 (m, 1H), 2.31-2.20 (m, 1H), 1.29-1.21 (m, 2H), 0.76-0.69 (m, 2H).

### Example 209: (R)-2-(3-(5-(3-Hydroxy-1-methyl-2-oxopyrrolidin-3-yl)isoxazol-3-yl)phenyl)-5-(methylamino)thiazole-4-carboxamide

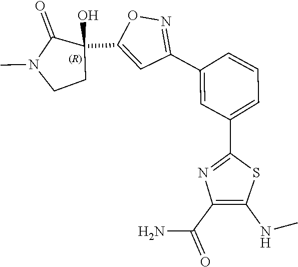

Step A: Ethyl 5-(methylamino)thiazole-4-carboxylate. 1,8-Diazabicyclo[5.4.0]undec-7-ene (10 mL, 68 mmol) was added to a mixture consisting of ethyl 5-bromothiazole-4-carboxylate (4.0 g, 17 mmol), methanamine hydrochloride (1.3 g, 19 mmol), and MeCN (30 mL). The resulting mixture was heated at 80° C. for 1 h via microwave irradiation. The reaction vessel was removed from the microwave heating device and allowed to gradually cool to rt. The mixture was concentrated to dryness in vacuo, and the residue subjected to HPLC (Xtimate C18 column, 5 μm, 150×40 mm, 11-41% MeCN/water with 0.05% NH₃H₂O) to give, after lyophilization, ethyl 5-(methylamino)thiazole-4-carboxylate as a yellow solid (560 mg, 15%). MS (ESI): Mass calcd. for C₇H₁₀N₂O₂S 186.05 m/z, found 187.1 [M+H]⁺.

Step B: 5-(Methylamino)thiazole-4-carboxylic acid. Sodium hydroxide (8.7 mL, 17 mmol, 2 M in H₂O) was added to a mixture consisting of ethyl 5-(methylamino)thiazole-4-carboxylate (540 mg, 2.90 mmol) and EtOH (5 mL). The resulting mixture was stirred for 16 h at room temperature. After this time, the mixture was acidified to pH=2 with 1 N aqueous HCl (10 mL) and extracted with EtOAc (20 mL×2). The combined organic extracts were washed with brine, dried over anhydrous Na₂SO₄, filtered, and concentrated in vacuo to afford 5-(methylamino)thiazole-4-carboxylic acid as a colorless oil (318 mg, 58%). MS (ESI): Mass calcd. for C₅H₆N₂O₂S 158.01 m/z, found 158.6 [M+H]⁺.

Step C: 5-(Methylamino)thiazole-4-carboxamide. HATU (0.92 g, 2.41 mmol) was added to a mixture consisting of 5-(methylamino)thiazole-4-carboxylic acid (318 mg, 2.01 mmol), TEA (0.84 mL, 6.0 mmol, 0.725 g/mL), and DMF (6 mL). The resultant mixture was stirred for 30 min at room temperature, treated with NH₄Cl (1.08 g, 20.1 mmol), and stirred for 16 h at room temperature. After this time, the mixture was poured into water (20 mL) and extracted with EtOAc (20 mL×3). The combined organic extracts were washed with brine (20 mL), dried over anhydrous Na₂SO₄, filtered, and concentrated to dryness in vacuo. The residue was subjected to silica gel chromatography (0-100% EtOAc/petroleum ether) to give 5-(methylamino)thiazole-4-carboxamide as a yellow solid (220 mg, 38%). MS (ESI): Mass calcd. for C₅H₇N₃OS 157.03 m/z, found 157.7 [M+H]⁺.

Step D: 2-Bromo-5-(Methylamino)thiazole-4-carboxamide. 1-Bromopyrrolidine-2,5-dione (125 mg, 0.700 mmol) was added to a solution consisting of 5-(methylamino)thiazole-4-carboxamide (100 mg, 0.636 mmol) in MeCN (2 mL). The resultant mixture was stirred at 25° C. for 1 h. After this time, the mixture was poured into water (10 mL) and extracted with EtOAc (10 mL×3). The combined organic extracts were washed with brine (10 mL), dried over anhydrous Na₂SO₄, filtered, and concentrated to dryness in vacuo. The residue was subjected to silica gel chromatography (0-100% EtOAc/petroleum ether) to afford 2-bromo-5-(methylamino)thiazole-4-carboxamide as a gray solid (80 mg, 53%). MS (ESI): Mass calcd. for C₅H₆BrN₃OS 234.94 m/z, found 235.9 [M+1]⁺.

Step E: (R)-2-(3-(5-(3-Hydroxy-1-methyl-2-oxopyrrolidin-3-yl)isoxazol-3-yl)phenyl)-5-(methylamino)thiazole-4-carboxamide. 1,1′-Bis(di-tert-butylphosphino)ferrocene palladium dichloride (22 mg, 0.034 mmol) was added to a mixture consisting of 2-bromo-5-(methylamino)thiazole-4-carboxamide (80 mg, 0.34 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 104 mg, 0.271 mmol), K₃PO₄ (216 mg, 1.02 mmol) and 1,4-dioxane/H₂O (2 mL, v/v=4:1) under a N₂ atmosphere. The resultant mixture was sparged with N₂ for 5 min, and then heated for 1 hour at 80° C. via microwave irradiation. The reaction vessel was removed from the microwave heating device and allowed to gradually cool to rt. The mixture was poured into water (10 mL) and extracted with EtOAc (10 mL×3). The combined organic extracts were washed with brine (10 mL), dried over anhydrous Na₂SO₄, filtered, and concentrated to dryness in vacuo. The residue was subjected to HPLC (Welch Xtimate C18 column, 5 m, 150×30 mm; 30-60% MeCN/water with 0.05% NH₃H₂O+10 mM NH₄HCO₃), and then subjected to SFC (DAICEL CHIRALCEL® OJ column, 10 μm, 250 mm×30 mm; 40% EtOH (containing 0.1% of NH₃H₂O)/supercritical CO₂), to give, after lyophilization, (R)-2-(3-(5-(3-hydroxy-1-methyl-2-oxopyrrolidin-3-yl)isoxazol-3-yl)phenyl)-5-(methylamino)thiazole-4-carboxamide as a colorless solid (10.4 mg, 7%). MS (ESI): Mass calcd. for C₁₉H₁₉N₅O₄S 413.12 m/z, found 414.2 [M+1]⁺. ¹H NMR (400 MHz, DMSO-d₆) δ 8.31-8.26 (m, 1H), 7.93-7.89 (m, 1H), 7.89-7.85 (m, 2H), 7.61-7.54 (m, 1H), 7.43 (s, 1H), 7.20-7.11 (m, 2H), 6.75 (s, 1H), 3.51-3.41 (m, 2H), 2.99 (d, J=4.9 Hz, 3H), 2.84 (s, 3H), 2.61-2.55 (m, 1H), 2.31-2.24 (m, 1H).

### Example 210: (R)-2-(3-(5-(3-Hydroxy-1-methyl-2-oxopyrrolidin-3-yl)isoxazol-3-yl)phenyl)-5-(oxetan-3-ylamino)thiazole-4-carboxamide

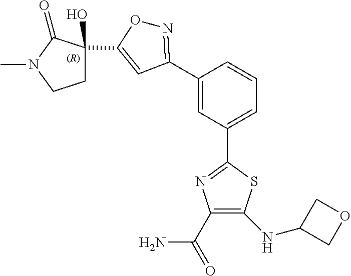

Step A: 5-Bromothiazole-4-carboxamide. Ethyl 5-bromothiazole-4-carboxylate (2.0 g, 8.5 mmol) and ammonia hydrate (20 mL) were added to a 100 mL round-bottomed flask, and the resulting mixture stirred for 3 h at 40° C. After this time, the reaction mixture was filtered through a pad of diatomaceous earth, and the filtrate concentrated to dryness in vacuo to give 5-bromothiazole-4-carboxamide as a yellow solid (1.4 g). MS (ESI): Mass calcd. for C₄H₃BrN₂OS 205.91 m/z, found 206.7 [M+H]⁺.

Step B: 5-(Oxetan-3-ylamino)thiazole-4-carboxamide. 5-Bromothiazole-4-carboxamide (2.0 g, 9.7 mmol), oxetan-3-amine (2.0 mL, 29 mmol), Cs₂CO₃ (6.3 g, 19 mmol), and 1,4-dioxane (60 mL) were added to a 250 mL three port flask. The resulting mixture was sparged with N₂ for 5 min, charged with RuPhos-Pd-G3 (80 mg, 0.968 mmol), and heated for 16 h at 80° C. After this time, the reaction vessel was removed from the oil bath and allowed to gradually cool to room temperature. The reaction was then diluted with water (40 mL), extracted with ethyl acetate (70 mL×3), and the combined extracts were treated with brine (50 mL), dried over anhydrous Na₂SO₄, filtered, and concentrated to dryness in vacuo to give a yellow solid. The yellow solid was then subjected to HPLC purification (Boston Uni C18 column, 5 μm, 150×40 mm; 10-40% ACN/water with 0.225% FA) to give, after lyophilization, 5-(oxetan-3-ylamino)thiazole-4-carboxamide as a yellow solid (350 mg, 5%). MS (ESI): Mass calcd. for C₇H₉N₃O₂S 199.04 m/z, found 199.9 [M+1]⁺.

Step C: 2-Bromo-5-(oxetan-3-ylamino)thiazole-4-carboxamide. 5-(Oxetan-3-ylamino)thiazole-4-carboxamide (240 mg, 1.21 mmol) and ACN (10 mL) were added to a 50 mL round-bottomed flask, and charged with NBS (120 mg, 0.674 mmol) at 0° C. The resulting mixture was stirred for 15 min at 0° C., and then diluted with water (8 mL) and extracted with ethyl acetate (15 mL×3). The combined organic extracts were washed with brine (20 mL), dried over anhydrous Na₂SO₄, filtered, and concentrated to dryness in vacuo to give a white solid. The white solid was then subjected to HPLC (Boston Uni C18 column, 5 μm, 150×40 mm; 8-38% ACN/water with 0.225% FA) to give, after lyophilization, 2-bromo-5-(oxetan-3-ylamino)thiazole-4-carboxamide as a white solid (70 mg, 21%). MS (ESI): Mass calcd. for C₇H₈BrN₃O₂S 276.95 m/z, found 277.8 [M+1]⁺. ¹H NMR (400 MHz, DMSO-d₆) δ 8.41-8.33 (m, 1H), 7.37-7.21 (m, 2H), 4.81-4.73 (m, 2H), 4.58-4.52 (m, 2H), 4.50-4.40 (m, 1H).

Step D: (R)-2-(3-(5-(3-Hydroxy-1-methyl-2-oxopyrrolidin-3-yl)isoxazol-3-yl)phenyl)-5-(oxetan-3-ylamino)thiazole-4-carboxamide. 2-Bromo-5-(oxetan-3-ylamino)thiazole-4-carboxamide (60 mg, 0.058 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 34 mg, 0.088 mmol), K₃PO₄ (36 mg, 0.17 mmol), and 1,4-dioxane/H₂O (1.8 mL, v/v=4:1) were added to a 5 mL tube, which was subsequently evacuated and refilled with N₂ (×3), and then charged with Pd(dtbpf)Cl₂ (6 mg, 0.009 mmol). The resulting mixture was heated for 1 h at 80° C. The reaction vessel was removed from the oil bath and allowed to gradually cool to rt. After this time, the mixture was diluted with water (3 mL) and extracted with ethyl acetate (5 mL×3). The combined organic extracts were washed with brine (4 mL), dried over anhydrous Na₂SO₄, filtered, and concentrated to dryness in vacuo to give a white solid. The white solid was then subjected to HPLC (Welch Xtimate C18 column, 5 μm, 150×30 mm; 27-57% MeCN/H₂O with 0.05% NH₃H₂O+10 mM NH₄HCO₃)) to give, after lyophilization, the title compound as a colorless solid (3.6 mg, 13%). MS (ESI): Mass calcd. for C₂₁H₂₁N₅O₅S 455.13 m/z, found 456.2 [M+1]⁺. ¹H NMR (400 MHz, DMSO-d₆): 8.40-8.34 (m, 1H), 8.31-8.22 (m, 1H), 7.94-7.86 (m, 2H), 7.62-7.53 (m, 2H), 7.36-7.29 (m, 1H), 7.12 (s, 1H), 6.78-6.68 (m, 1H), 4.91-4.78 (m, 2H), 4.66-4.50 (m, 3H), 3.54-3.41 (m, 2H), 2.84 (s, 3H), 2.62-2.56 (m, 1H), 2.33-2.22 (m, 1H).

### Example 211: (R)-2-(3-(5-(3-Hydroxy-1-methyl-2-oxopyrrolidin-3-yl)isoxazol-3-yl)phenyl)-5-((1-methyl-1H-pyrazol-4-yl)amino)thiazole-4-carboxamide

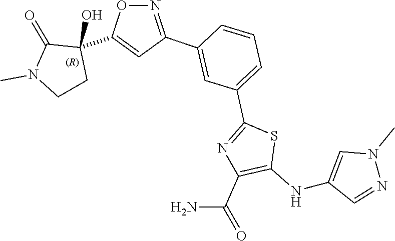

Step A: Ethyl 5-((1-methyl-1H-pyrazol-4-yl)amino)thiazole-4-carboxylate. 1,8-diazabicyclo[5.4.0]undec-7-ene (5.1 mL, 34 mmol) was added to a mixture consisting of ethyl 5-bromothiazole-4-carboxylate (2.0 g, 8.5 mmol), 1-methyl-1H-pyrazol-4-amine (905 mg, 9.31 mmol), and MeCN (30 mL). The resulting mixture was heated at 80° C. via microwave irradiation. The reaction vessel was removed from microwave heating device and allowed to gradually cool to rt. The mixture was concentrated to dryness in vacuo, and the residue subjected to silica gel chromatography (0-30% EtOAc/petroleum ether) to afford ethyl 5-((1-methyl-1H-pyrazol-4-yl)amino)thiazole-4-carboxylate as a colorless solid (1.05 g, 47%).

Step B: Ethyl 2-bromo-5-((1-methyl-1H-pyrazol-4-yl)amino)thiazole-4-carboxylate. 1-Bromopyrrolidine-2,5-dione (705 mg, 3.96 mmol) was added to a solution consisting of ethyl 5-((1-methyl-1H-pyrazol-4-yl)amino)thiazole-4-carboxylate (1.0 g, 4.0 mmol) in MeCN (10 mL). The resulting mixture was heated at 70° C. for 16 h. The reaction vessel was removed from the oil bath and allowed to gradually cool to rt. The mixture was poured into water (30 mL) and extracted with EtOAc (30 mL×3). The combined organic extracts were washed with brine, dried over anhydrous Na₂SO₄, filtered, and concentrated to dryness in vacuo, and the residue subjected to silica gel chromatography (0-50% EtOAc/petroleum ether) to afford ethyl 2-bromo-5-((1-methyl-1H-pyrazol-4-yl)amino)thiazole-4-carboxylate as a yellow solid (872 mg, 63%). MS (ESI): Mass calcd. for C₁₀H₁₁BrN₄O₂S 329.98 m/z, found 330.7 [M+H]⁺.

Step C: (R)-Ethyl 2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxylate. 1,1′-Bis(di-tert-butylphosphino)ferrocene palladium dichloride (79 mg, 0.12 mmol) was added to a mixture consisting of ethyl 2-bromo-5-((1-methyl-1H-pyrazol-4-yl)amino)thiazole-4-carboxylate (400 mg, 1.21 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 464 mg, 1.21 mmol), K₃PO₄ (769 mg, 3.62 mmol) and 1,4-dioxane/H₂O (8 mL, v/v=4:1) under a N₂ atmosphere. The resulting mixture was heated for 1 hour at 80° C. via microwave irradiation. The reaction vessel was removed from the microwave heating device and allowed to gradually cool to rt. The mixture was poured into water (10 mL) and extracted with EtOAc (10 mL×3). The combined organic extracts were washed with brine (10 mL), dried over anhydrous Na₂SO₄, filtered, and concentrated to dryness in vacuo, and the residue subjected to silica gel chromatography (0-100% EtOAc/petroleum ether) to afford (R)-ethyl 2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxylate as a yellow solid (136 mg, 18%). MS (ESI): Mass calcd. for C₂₄H₂₄N₆O₅S 508.15 m/z, found 509.1 [M+H]⁺.

Step D: (R)—N-(2,4-Dimethoxybenzyl)-2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxamide. Trimethylaluminum (0.2 mL, 2 mmol) was added to a mixture consisting of (R)-ethyl 2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxylate (100 mg, 0.197 mmol), (2,4-dimethoxyphenyl)methanamine (89 μL, 0.59 mmol) and toluene (1 mL). The resulting mixture was heated for 2 hours at 70° C. After this time, the reaction vessel was removed from the oil bath and allowed to gradually cool to rt. The mixture was quenched with MeOH (2 mL) and concentrated to dryness in vacuo. The residue was subjected to silica gel chromatography (0-100% EtOAc/petroleum ether) to afford (R)—N-(2,4-dimethoxybenzyl)-2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxamide as a yellow solid (93 mg, 45%). MS (ESI): Mass calcd. for C₃₁H₃₁N₇O₆S 629.21 m/z, found 630.1 [M+1]⁺.

Step E: (R)-2-(3-(5-(3-Hydroxy-1-methyl-2-oxopyrrolidin-3-yl)isoxazol-3-yl)phenyl)-5-((1-methyl-1H-pyrazol-4-yl)amino)thiazole-4-carboxamide. 2,2,2-Trifluoroacetic acid (189 μL, 32.54 mmol) was added to a mixture consisting of (R)—N-(2,4-dimethoxybenzyl)-2-(3-(5-(3-hydroxy-1-methyl-2-oxopyrrolidin-3-yl)isoxazol-3-yl)phenyl)-5-((1-methyl-1H-pyrazol-4-yl)amino)thiazole-4-carboxamide (80 mg, 0.13 mmol) in DCM (1 mL) and the resulting mixture stirred for 16 h at room temperature. After this time, the mixture was concentrated to dryness in vacuo. The residue was subjected to HPLC (Phenomenex Gemini-NX C18 column, 3 m, 75×30 mm; 25-55% MeCN/water with 0.05% NH₃H₂O+10 mM NH₄HCO₃) to give, after lyophilization, the title compound as a yellow solid (10.1 mg, 16%). MS (ESI): Mass calcd. for C₂₂H₂₁N₇O₄S 479.14 m/z, found 480.2 [M+1]⁺. ¹H NMR (400 MHz, DMSO-d₆) δ 9.61 (s, 1H), 8.29 (s, 1H), 7.97-7.93 (m, 2H), 7.89 (d, J=7.9 Hz, 1H), 7.65 (s, 1H), 7.62-7.56 (m, 2H), 7.41 (s, 1H), 7.13 (s, 1H), 6.74 (s, 1H), 3.85 (s, 3H), 3.51-3.45 (m, 2H), 2.84 (s, 3H), 2.61-2.57 (m, 1H), 2.32-2.23 (m, 1H).

### Example 212: (R)-2-(3-(5-(3-hydroxy-1-methyl-2-oxopyrrolidin-3-yl)isoxazol-3-yl)phenyl)thiazole-5-carboxamide

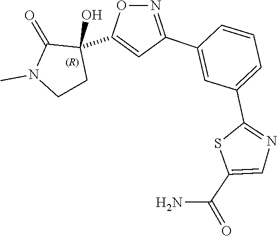

1,1′-Bis(di-tert-butylphosphino)ferrocene palladium dichloride (31 mg, 0.048 mmol) was added to a mixture consisting of 2-bromothiazole-5-carboxamide (100 mg, 0.483 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 186 mg, 0.483 mmol), K₃PO₄ (308 mg, 1.45 mmol), and 1,4-dioxane/H₂O=4:1 (2 mL) under a N₂ atmosphere. The resulting mixture was stirred for 1 hour at 80° C. via microwave irradiation. The reaction vessel was removed from the microwave heating device and allowed to gradually cool to rt. The mixture was concentrated to dryness in vacuo, and the residue subjected to HPLC (Welch Xtimate C18 column, 5 μm, 150×30 mm; 17-47% MeCN/water with 0.05% NH₃H₂O+10 mM NH₄HCO₃) to give, after lyophilization, the title compound (98 mg, 53%) as a colorless solid. MS (ESI): Mass calcd. for C₁₈H₁₆N₄O₄S 384.09 m/z, found 385.0 [M+1]⁺. ¹H NMR (400 MHz, DMSO-d₆) δ 8.49 (s, 1H), 8.46-8.42 (m, 1H), 8.26 (s, 1H), 8.11 (d, J=7.7 Hz, 1H), 8.04 (d, J=7.9 Hz, 1H), 7.77-7.63 (m, 2H), 7.18 (s, 1H), 6.76 (s, 1H), 3.50-3.42 (m, 2H), 2.85 (s, 3H), 2.63-2.54 (m, 1H), 2.33-2.23 (m, 1H).

### Example 213: (R)-2-(3-(5-(3-Hydroxy-1-methyl-2-oxopyrrolidin-3-yl)isoxazol-3-yl)phenyl)-5-((2-methoxypyridin-3-yl)amino)thiazole-4-carboxamide

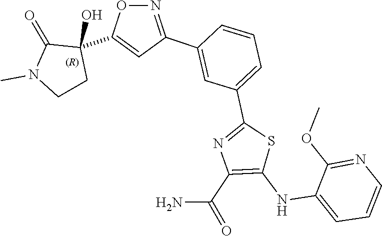

Step A: Ethyl 5-((2-methoxypyridin-3-yl)amino)thiazole-4-carboxylate. A mixture consisting of ethyl 5-chlorothiazole-4-carboxylate (1.00 g, 5.22 mmol), 2-methoxypyridin-3-amine (1.26 g, 0.180 mmol), CsF (1.59 g, 10.4 mmol), and DMF (10 mL) was heated at 150° C. for 4 h via microwave irradiation. The reaction vessel was removed from the microwave heating device and allowed to gradually cool to rt. The mixture was filtered, and the filter cake washed with EtOAc (25 mL). The filtrate was concentrated in vacuo to give a brown gum. The brown gum was subjected to silica gel chromatography (0-50% EtOAc/petroleum ether) to afford ethyl 5-((2-methoxypyridin-3-yl)amino)thiazole-4-carboxylate as an off-yellow solid (1.10 g, 74.3%). MS (ESI): Mass calcd. for C₁₂H₁₃N₃O₃S 279.07 m/z, found 279.7 [M+H]⁺.

Step B: Ethyl 2-bromo-5-((2-methoxypyridin-3-yl)amino)thiazole-4-carboxylate. Ethyl 5-((2-methoxypyridin-3-yl)amino)thiazole-4-carboxylate (1.00 g, 3.58 mmol) and DMF (10.0 mL) were added to a 40 mL vial and the resulting mixture cooled to 0° C. (ice/water), treated with NBS (796 mg, 4.48 mmol), and stirred at 0° C. for 2 h. After this time, the mixture was concentrated to dryness in vacuo, and the residue was subjected to silica gel chromatography (0-30% EtOAc/petroleum ether) to afford ethyl 2-bromo-5-((2-methoxypyridin-3-yl)amino)thiazole-4-carboxylate as an off-white solid (650 mg, 46%). MS (ESI): Mass calcd. for C₁₂H₁₂BrN₃O₃S 355.98 m/z, found 356.1 [M+H]⁺.

Step C: (R)-Ethyl 2-(3-(5-(3-hydroxy-1-methyl-2-oxopyrrolidin-3-yl)isoxazol-3-yl)phenyl)-5-((2-methoxypyridin-3-yl)amino)thiazole-4-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 107 mg, 0.279 mmol), Pd(dtbpf)Cl₂ (18.2 mg, 0.028 mmol), K₃PO₄ (0.558 mL, 0.558 mmol, 1 N in H₂O), and 1,4-dioxane (1.50 mL) were added to a 8 mL vial, which was subsequently evacuated and refilled with Ar (×3). The resulting mixture was treated with a solution of ethyl 2-bromo-5-((2-methoxypyridin-3-yl)amino)thiazole-4-carboxylate (100 mg, 0.279 mmol) in 1,4-dioxane (1.50 mL) dropwise over 1.5 h at 85° C., and then the resulting mixture was heated at 85° C. for 1 h. After this time, the reaction vessel was removed from the heating device and allowed to gradually cool to rt. The mixture was filtered, and the filtrate concentrated in vacuo to give a yellow oil. The oil was subjected to silica gel chromatography (0-15% MeOH/CH₂Cl₂) to afford (R)-ethyl 2-(3-(5-(3-hydroxy-1-methyl-2-oxopyrrolidin-3-yl)isoxazol-3-yl)phenyl)-5-((2-methoxypyridin-3-yl)amino)thiazole-4-carboxylate (240 mg, 80%) as a yellow oil. LCMS (ESI): Mass calcd. for C₂₆H₂₅N₅O₆S 535.15 m/z, found 535.9 [M+H]⁺.

Step D: (R)-2-(3-(5-(3-Hydroxy-1-methyl-2-oxopyrrolidin-3-yl)isoxazol-3-yl)phenyl)-5-((2-methoxypyridin-3-yl)amino)thiazole-4-carboxamide. (R)-Ethyl 2-(3-(5-(3-hydroxy-1-methyl-2-oxopyrrolidin-3-yl)isoxazol-3-yl)phenyl)-5-((2-methoxypyridin-3-yl)amino)thiazole-4-carboxylate (145 mg, 0.271 mmol) and 7 M NH₃ in MeOH (15 mL) was added to a 40 mL vial. The resulting mixture was heated at 75° C. for 16 h. The reaction vessel was removed from heat and allowed to gradually cool to rt. The mixture was filtered, and the filtrate concentrated in vacuo to give a yellow oil. The yellow oil was then subjected to silica gel chromatography (0-15% MeOH/CH₂Cl₂), and then subjected to HPLC (Welch Xtimate C18 column, 5 μm, 150×30 mm; 37-67% MeCN/water with 0.05% NH₃H₂O+10 mM NH₄HCO₃) to give, after lyophilization, (R)-2-(3-(5-(3-hydroxy-1-methyl-2-oxopyrrolidin-3-yl)isoxazol-3-yl)phenyl)-5-((2-methoxypyridin-3-yl)amino)thiazole-4-carboxamide as a yellow solid (31.5 mg, 16%). MS (ESI): Mass calcd. for C₂₄H₂₂N₆O₅S 506.14 m/z, found 507.2 [M+H]⁺. ¹H NMR (400 MHz, DMSO-d₆) δ 11.03 (s, 1H), 8.40 (s, 1H), 8.08 (d, J=7.6 Hz, 1H), 7.98-7.88 (m, 2H), 7.86-7.81 (m, 1H), 7.77 (d, J=7.6 Hz, 1H), 7.67-7.59 (m, 2H), 7.16 (s, 1H), 7.13-7.07 (m, 1H), 6.75 (s, 1H), 4.01 (s, 3H), 3.53-3.40 (m, 2H), 2.85 (s, 3H), 2.62-2.54 (m, 1H), 2.35-2.25 (m, 1H). ¹H NMR (400 MHz, MeOH-d₄) δ 8.38 (s, 1H), 8.00 (d, J=7.2 Hz, 1H), 7.87 (d, J=7.2 Hz, 1H), 7.78 (d, J=4.4 Hz, 1H), 7.71 (d, J=7.6 Hz, 1H), 7.63-7.51 (m, 1H), 7.15-6.94 (m, 2H), 4.08 (s, 3H), 3.69-3.53 (m, 2H), 3.00 (s, 3H), 2.84-2.67 (m, 1H), 2.53-2.35 (m, 1H).

### Example 214: (R)-2-Bromo-5-(3-(5-(3-hydroxy-1-methyl-2-oxopyrrolidin-3-yl)isoxazol-3-yl)phenyl)thiazole-4-carboxamide

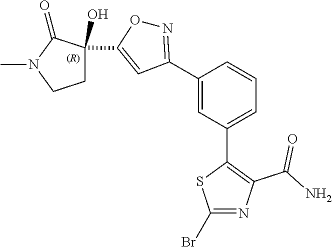

Step A: 2,5-Dibromothiazole-4-carboxamide. Methyl 2,5-dibromothiazole-4-carboxylate (2.0 g, 6.6 mmol) and NH₃ (40 mL, 7 M in MeOH) were added to a 250 mL three port flask, and the resulting mixture stirred for 1.5 h at 50° C. After this time, the reaction vessel was removed from the oil bath and allowed to gradually cool to room temperature. The reaction mixture was filtered, and the filtrate concentrated to dryness in vacuo to give 2,5-dibromothiazole-4-carboxamide as a white solid (800 mg). MS (ESI): Mass calcd. for C₄H₂Br₂N₂OS 283.83 m/z found 284.6 [M+H]⁺.

Step B: (R)-5-Bromo-2-(3-(5-(3-hydroxy-1-methyl-2-oxopyrrolidin-3-yl)isoxazol-3-yl)phenyl)thiazole-4-carboxamide. 2,5-Dibromothiazole-4-carboxamide (714 mg, 2.50 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 500 mg, 1.30 mmol), K₂CO₃ (1.0 g, 7.74 mmol), and 1,4-dioxane/H₂O (4:1) (20 mL) were added to a 100 mL three port flask, which was subsequently evacuated and refilled with N₂ (×3), and charged with Pd(dppf)Cl₂ (186 mg, 0.254 mmol). The resulting mixture was stirred for 3 h at 50° C. The reaction mixture was cooled to room temperature, diluted with water (10 mL), and extracted with ethyl acetate (25 mL×3). The combined extracts were washed with brine (20 mL), dried over anhydrous Na₂SO₄, filtered, and concentrated to dryness in vacuo to give a white solid, which was subjected to HPLC (Xtimate C18 column, 5 μm, 150×40 mm; 25-55% MeCN/H₂O with 0.05% NH₃H₂O) to give (R)-5-bromo-2-(3-(5-(3-hydroxy-1-methyl-2-oxopyrrolidin-3-yl)isoxazol-3-yl)phenyl)thiazole-4-carboxamide as a white solid (10 mg, 7%). MS (ESI): Mass calcd. for C₁₈H₁₅BrN₄O₄S 462.00 m/z found 462.7 [M+H]⁺. ¹H NMR (400 MHz, DMSO-d₆) δ 8.47 (s, 1H), 8.17-8.09 (m, 2H), 8.08-8.02 (m, 1H), 7.77 (br s, 1H), 7.72-7.63 (m, 1H), 7.19 (s, 1H), 6.76 (s, 1H), 3.51-3.41 (m, 2H), 2.85 (s, 3H), 2.62-2.54 (m, 1H), 2.33-2.22 (m, 1H).

Step C: (R)-2-Bromo-5-(3-(5-(3-hydroxy-1-methyl-2-oxopyrrolidin-3-yl)isoxazol-3-yl)phenyl)thiazole-4-carboxamide. (R)-5-Bromo-2-(3-(5-(3-hydroxy-1-methyl-2-oxopyrrolidin-3-yl)isoxazol-3-yl)phenyl)thiazole-4-carboxamide (20 mg, 0.043 mmol) was subjected to SFC (DAICEL CHIRALCEL® OD-H column, 5 μm, 250×30 mm; 45-55% (v/v) EtOH (containing 0.1% NH₃H₂O)/CO₂)) to give two products. First eluting: title compound as a colorless solid (4 mg, 20%). LC-MS (ESI): RT=1.38 min, MS (ESI): Mass calcd. for C₁₈H₁₅BrN₄O₄S 462.00 m/z found 463.0 [M+H]⁺. ¹H NMR (400 MHz, DMSO-d₆) δ 8.05-8.02 (m, 1H), 7.96-7.91 (m, 1H), 7.90-7.87 (m, 1H), 7.69-7.63 (m, 2H), 7.61-7.54 (m, 1H), 7.04 (s, 1H), 6.75 (s, 1H), 3.49-3.43 (m, 2H), 2.84 (s, 3H), 2.59-2.55 (m, 1H), 2.34-2.21 (m, 1H).

### Example 215: (R)-5-Bromo-2-(3-(5-(3-hydroxy-1-methyl-2-oxopyrrolidin-3-yl)isoxazol-3-yl)phenyl)thiazole-4-carboxamide

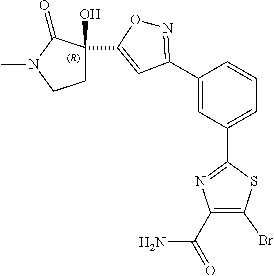

(R)-5-Bromo-2-(3-(5-(3-hydroxy-1-methyl-2-oxopyrrolidin-3-yl)isoxazol-3-yl)phenyl)thiazole-4-carboxamide was isolated during the purification of (R)-2-bromo-5-(3-(5-(3-hydroxy-1-methyl-2-oxopyrrolidin-3-yl)isoxazol-3-yl)phenyl)thiazole-4-carboxamide (Example 214), eluting out as the second compound, yielding the title compound as a colorless solid (10 mg, 49%). MS (ESI): Mass calcd. for C₁₈H₁₅BrN₄O₄S 462.00 m/z found 463.1 [M+H]⁺. ¹H NMR (400 MHz, DMSO-d₆) δ 8.49-8.45 (m, 1H), 8.17-8.10 (m, 2H), 8.08-8.03 (m, 1H), 7.77 (s, 1H), 7.71-7.66 (m, 1H), 7.19 (s, 1H), 6.77 (s, 1H), 3.50-3.42 (m, 2H), 2.85 (s, 3H), 2.60-2.54 (m, 1H), 2.35-2.24 (m, 1H).

### Example 216: (R)-5-((1-Acetylazetidin-3-yl)amino)-2-(3-(5-(3-hydroxy-1-methyl-2-oxopyrrolidin-3-yl)isoxazol-3-yl)phenyl)thiazole-4-carboxamide

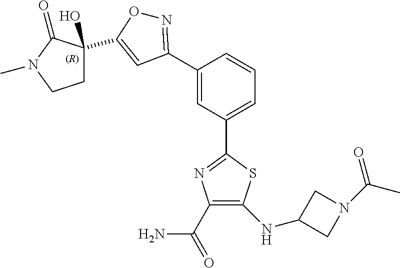

Step A: 5-Chlorothiazole-4-carboxamide. A mixture consisting of ethyl 5-chlorothiazole-4-carboxylate (1650 mg, 8.6 mmol), and NH₄OH (10 mL, 7 M in MeOH) was stirred for 4 h at 35° C. After this time, the mixture was filtered. The filter cake was washed with H₂O (50 mL) and dried in vacuo to give 5-chlorothiazole-4-carboxamide as a light yellow solid (1.0 g, 71%). ¹H NMR (400 MHz, DMSO-d₆) δ 9.04 (s, 1H), 7.81 (br s, 1H), 7.67 (br s, 1H).

Step B: 5-((1-Acetylazetidin-3-yl)amino)thiazole-4-carboxamide. 5-Chlorothiazole-4-carboxamide (500 mg, 3.1 mmol), 1-(3-aminoazetidin-1-yl)ethanone (695 mg, 4.6 mmol), Cs₂CO₃ (2.5 g, 7.7 mmol), and MeCN (10 mL) were added to a 20 mL vial. The resulting mixture was heated at 80° C. for 20 h. After this time, the reaction vessel was removed from the oil bath and allowed to gradually cool to rt. The mixture was combined with other 3 batches, filtered, and the filtrate concentrated to dryness in vacuo to give a brown gum, which was subjected to silica gel chromatography (0-10% MeOH/CH₂Cl₂) to afford 5-((1-acetylazetidin-3-yl)amino)thiazole-4-carboxamide (500 mg, 17%) as a light brown solid. MS (ESI): Mass calcd. for C₉H₁₂N₄O₂S 240.07 m/z, found 240.6 [M+H]⁺.

Step C: 5-((1-Acetylazetidin-3-yl)amino)-2-iodothiazole-4-carboxamide. A mixture consisting of 5-((1-acetylazetidin-3-yl)amino)thiazole-4-carboxamide (250 mg, 1.04 mmol) and ACN (10 mL) was treated with NIS (272 mg, 1.04 mmol) in one portion. The resulting mixture was stirred at room temperature for 30 min. After this time, the mixture was concentrated in vacuo to give an orange solid, which was subjected to silica gel chromatography (0-10% MeOH/CH₂Cl₂) to afford 5-((1-acetylazetidin-3-yl)amino)-2-iodothiazole-4-carboxamide (150 mg, 39%) as a light brown solid.

Step D: (R)-5-((1-Acetylazetidin-3-yl)amino)-2-(3-(5-(3-hydroxy-1-methyl-2-oxopyrrolidin-3-yl)isoxazol-3-yl)phenyl)thiazole-4-carboxamide. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 126 mg, 0.330 mmol), Pd(dtbpf)Cl₂ (36 mg, 0.055 mmol), K₃PO₄ (1.1 mL, 1.1 mmol, 1 M in H₂O), 5-((1-acetylazetidin-3-yl)amino)-2-iodothiazole-4-carboxamide (200 mg, 0.068 mmol, in 5 mL of 1,4-dioxane), and 1,4-dioxane (5 mL) were added to a 20 mL vial, which was subsequently evacuated and refilled with N₂ for 3 times. The resulting mixture was stirred for 2 h at 80° C. After this time, the reaction vessel was removed from the oil bath and allowed to gradually cool to rt. The mixture was filtered through a pad of diatomaceous earth, and the pad washed with EtOAc (25 mL). The filtrate was concentrated in vacuo to give a brown gum, which was subjected to HPLC (Boston Green ODS column, 5 μm, 150×30 mm; 20-50% (v/v) CH₃CN/H₂O with 0.225% FA) to give, after lyophilization, (R)-5-((1-acetylazetidin-3-yl)amino)-2-(3-(5-(3-hydroxy-1-methyl-2-oxopyrrolidin-3-yl)isoxazol-3-yl)phenyl)thiazole-4-carboxamide as an off-white solid (18.7 mg, 7%). MS (ESI): Mass calcd. for C₂₃H₂₄N₆O₅S 496.15 m/z, found 497.2 [M+H]⁺. ¹H NMR (400 MHz, DMSO-d₆) δ 8.35-8.23 (m, 2H), 7.93 (d, J=8.0 Hz, 1H), 7.89 (d, J=8.0 Hz, 1H), 7.65-7.54 (m, 2H), 7.32 (br s, 1H), 7.12 (s, 1H), 6.77 (s, 1H), 4.46 (d, J=7.6 Hz, 1H), 4.28-4.19 (m, 2H), 4.19-4.12 (m, 1H), 3.88-3.79 (m, 1H), 3.54-3.52 (m, 2H), 2.84 (s, 3H), 2.59 (br d, J=2.4 Hz, 1H), 2.32-2.23 (m, 1H), 1.78 (s, 3H). ¹H NMR (400 MHz, CD₃OD) δ 8.38-8.29 (m, 1H), 7.96-7.90 (m, 1H), 7.89-7.84 (m, 1H), 7.60-7.53 (m, 1H), 7.00 (s, 1H), 4.67-4.60 (m, 1H), 4.45-4.32 (m, 2H), 4.24 (dd, J=4.4, 8.8 Hz, 1H), 3.98 (dd, J=4.4, 9.6 Hz, 1H), 3.65-3.55 (m, 2H), 2.99 (s, 3H), 2.80-2.71 (m, 1H), 2.47-2.38 (m, 1H), 1.93 (s, 3H).

### Example 217: (R)-2-(3-(5-(3-Hydroxy-1-methyl-2-oxopyrrolidin-3-yl)isoxazol-3-yl)phenyl)-5-methylthiazole-4-carboxamide

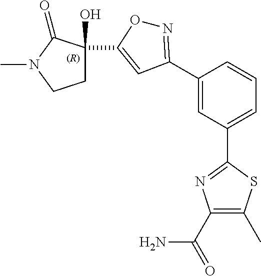

Step A: Ethyl 2-bromo-5-methylthiazole-4-carboxylate. NBS (3.12 g, 17.5 mmol) was added to a solution consisting of ethyl 5-methylthiazole-4-carboxylate (2.00 g, 11.7 mmol) in ACN (20 mL). The reaction was heated at 70° C. for 18 h. After this time, the reaction vessel was removed from the oil bath and allowed to gradually cool to room temperature. The reaction was quenched with sat. aqueous NaHSO₃ (30 mL) and extracted with EtOAc (30 mL×2). The combined organic extracts were washed with brine, dried over anhydrous Na₂SO₄, filtered, and concentrated to dryness in vacuo. The residue was subjected to silica gel chromatography (0-100% EtOAc/petroleum ether), and then subjected to HPLC (Xtimate C18 column, 5 μm, 150×40 mm; 31-61% CH₃CN/H₂O with 0.05% NH₃H₂O) to give, after lyophilization, ethyl 2-bromo-5-methylthiazole-4-carboxylate as a white solid (600 mg, 20.5%). MS (ESI): Mass calcd. for C₇H₈BrNO₂S 250.11 m/z, found 251.8 [M+1]⁺. ¹H NMR (400 MHz, CDCl₃) δ 4.45-4.34 (m, 2H), 2.71 (d, J=3.1 Hz, 3H), 1.45-1.34 (m, 3H).

Step B: (R)-Ethyl 2-(3-(5-(3-hydroxy-1-methyl-2-oxopyrrolidin-3-yl)isoxazol-3-yl)phenyl)-5-methylthiazole-4-carboxylate. Ethyl 2-bromo-5-methylthiazole-4-carboxylate (220 mg, 0.880 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 280 mg, 0.729 mmol), K₃PO₄ (390 mg, 1.84 mmol), Pd(dppf)Cl₂ (55 mg, 0.075 mmol), 1,4-dioxane (4 mL), and H₂O (1 mL) were added to a 8 mL vial. The resultant mixture was evacuated and refilled with N₂ for 3 times, and then heated at 100° C. for 12 h. The reaction vessel was removed from the oil bath and allowed to gradually cool to rt. The resultant mixture was diluted with H₂O (15 mL) and extracted with dichloromethane (15 mL×3). The combined organic extracts were washed with H₂O (10 mL×2), dried over anhydrous Na₂SO₄, filtered, and concentrated to dryness in vacuo. The residue was subjected to silica gel chromatography (0-100% EtOAc/petroleum ether) to give (R)-ethyl 2-(3-(5-(3-hydroxy-1-methyl-2-oxopyrrolidin-3-yl)isoxazol-3-yl)phenyl)-5-methylthiazole-4-carboxylate as a yellow solid (280 mg, 70.4%). MS (ESI): Mass calcd. for C₂₁H₂₁N₃O₅S 427.12 m/z, found 428.0 [M+1]⁺.

Step C: (R)-2-(3-(5-(3-Hydroxy-1-methyl-2-oxopyrrolidin-3-yl)isoxazol-3-yl)phenyl)-5-methylthiazole-4-carboxamide. (R)-Ethyl 2-(3-(5-(3-hydroxy-1-methyl-2-oxopyrrolidin-3-yl)isoxazol-3-yl)phenyl)-5-methylthiazole-4-carboxylate (260 mg, 0.608 mmol) was added into a solution of 7 M NH₃ in MeOH (26 mL, 182 mmol). The resultant mixture was stirred for 72 h at 50° C. After this time, the mixture was concentrated in vacuo. The residue was subjected to HPLC (Phenomenex Gemini-NX C18 column, 3 m, 75×30 mm; 24-54% (v/v) CH₃CN/H₂O with 0.04% NH₃H₂O+10 mM NH₄HCO₃) to give, after lyophilization, (R)-2-(3-(5-(3-hydroxy-1-methyl-2-oxopyrrolidin-3-yl)isoxazol-3-yl)phenyl)-5-methylthiazole-4-carboxamide as a white solid (106.8 mg, 43.9%). MS (ESI): Mass calcd. for C₁₉H₁₈N₄O₄S 398.10 m/z found 399.2 [M+H]⁺. ¹H NMR (400 MHz, DMSO-d₆) δ 8.44-8.48 (m, 1H) 8.05-8.10 (m, 1H) 7.96-8.02 (m, 2H) 7.63-7.69 (m, 1H) 7.58 (br s, 1H) 7.17 (s, 1H) 6.75 (s, 1H) 3.39-3.54 (m, 1H) 3.39-3.54 (m, 1H) 2.85 (s, 3H) 2.80 (s, 3H) 2.55-2.62 (m, 1H) 2.23-2.35 (m, 1H).

### Example 218: (R)-5-Ethyl-2-(3-(5-(3-hydroxy-1-methyl-2-oxopyrrolidin-3-yl)isoxazol-3-yl)phenyl)thiazole-4-carboxamide

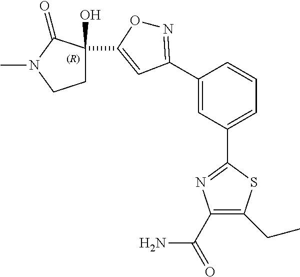

Step A: (R)-Methyl 5-ethyl-2-(3-(5-(3-hydroxy-1-methyl-2-oxopyrrolidin-3-yl)isoxazol-3-yl)phenyl)thiazole-4-carboxylate. 1,1′-Bis(di-tert-butylphosphino)ferrocene palladium dichloride (26 mg, 0.040 mmol) was added to a mixture consisting of methyl 2-bromo-5-ethylthiazole-4-carboxylate (100 mg, 0.400 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 154 mg, 0.400 mmol), K₃PO₄ (255 mg, 1.20 mmol), and 1,4-dioxane/H₂O=4:1 (2 mL) under N₂ atmosphere. The resulting mixture was stirred for 1 hour at 80° C. via microwave irradiation. The reaction vessel was removed from the microwave heating device and allowed to gradually cool to rt. The mixture was concentrated to dryness in vacuo, and the residue subjected to silica gel chromatography (0-100% EtOAc/petroleum ether) to afford (R)-methyl 5-ethyl-2-(3-(5-(3-hydroxy-1-methyl-2-oxopyrrolidin-3-yl)isoxazol-3-yl)phenyl)thiazole-4-carboxylate (150 mg, 85%) as a yellow oil. MS (ESI): Mass calcd. for C₂₁H₂₁N₃O₅S 427.12 m/z, found 428.1 [M+H]⁺.

Step B: (R)-5-Ethyl-2-(3-(5-(3-hydroxy-1-methyl-2-oxopyrrolidin-3-yl)isoxazol-3-yl)phenyl)thiazole-4-carboxamide. NH₃ in MeOH (4.0 mL, 28 mmol) was added to a solution of (R)-methyl 5-ethyl-2-(3-(5-(3-hydroxy-1-methyl-2-oxopyrrolidin-3-yl)isoxazol-3-yl)phenyl)thiazole-4-carboxylate (150 mg, 0.351 mmol) in MeOH (2 mL). The resulting mixture was stirred for 40 h at 50° C. After this time, the mixture was concentrated to dryness in vacuo, and the residue subjected to HPLC (Phenomenex Gemini-NX C18 column, 3 m, 75×30 mm; 30-60% MeCN/water with 0.04% NH₃H₂O+10 mM NH₄HCO₃) to give, after lyophilization, the title compound as a colorless solid (62 mg, 43%). MS (ESI): Mass calcd. for C₂₀H₂₀N₄O₄S 412.46 m/z, found 413.2 [M+1]⁺. ¹H NMR (400 MHz, DMSO-d₆) δ 8.49-8.44 (m, 1H), 8.10 (d, J=7.9 Hz, 1H), 8.04-7.95 (m, 2H), 7.69-7.62 (m, 1H), 7.58 (s, 1H), 7.17 (s, 1H), 6.75 (s, 1H), 3.53-3.41 (m, 2H), 3.34-3.31 (m, 2H), 2.85 (s, 3H), 2.61-2.54 (m, 1H), 2.32-2.24 (m, 1H), 1.32-1.27 (m, 3H).

### Example 219: (R)-2-(3-(5-(3-Hydroxy-1-methyl-2-oxopyrrolidin-3-yl)isoxazol-3-yl)phenyl)-5-isopropylthiazole-4-carboxamide

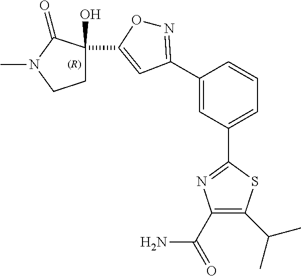

Step A: (R)-Methyl 2-(3-(5-(3-hydroxy-1-methyl-2-oxopyrrolidin-3-yl)isoxazol-3-yl)phenyl)-5-isopropylthiazole-4-carboxylate. Methyl 2-bromo-5-isopropylthiazole-4-carboxylate (100 mg, 0.379 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 189 mg, 0.472 mmol), K₃PO₄ (241 mg, 1.14 mmol), and 1,4-dioxane/H₂O (4:1) (2 mL) were added to a 25 mL three port flask, which was subsequently evacuated and refilled with N₂ (×3), and charged with Pd(dtbpf)Cl₂ (25 mg, 0.038 mmol). The resulting mixture was stirred for 1 h at 90° C. The reaction vessel was removed from the oil bath and allowed to gradually cool to rt. The reaction was then diluted with water (3 mL), extracted with ethyl acetate (5 mL×3), and the combined extracts were washed with brine (4 mL), dried over anhydrous Na₂SO₄, filtered, and concentrated to dryness in vacuo to give a yellow solid. The yellow solid was subjected to silica gel chromatography (0-100% ethyl acetate/petroleum ether) to give (R)-methyl 2-(3-(5-(3-hydroxy-1-methyl-2-oxopyrrolidin-3-yl)isoxazol-3-yl)phenyl)-5-isopropylthiazole-4-carboxylate as a white solid (150 mg, 76%). MS (ESI): Mass calcd. for C₂₂H₂₃N₃O₅S 441.14 m/z, found 442.1 [M+1]⁺.

Step B: (R)-2-(3-(5-(3-Hydroxy-1-methyl-2-oxopyrrolidin-3-yl)isoxazol-3-yl)phenyl)-5-isopropylthiazole-4-carboxamide. (R)-Methyl 2-(3-(5-(3-hydroxy-1-methyl-2-oxopyrrolidin-3-yl)isoxazol-3-yl)phenyl)-5-isopropylthiazole-4-carboxylate (135 mg, 0.306 mmol) and NH₃ (5.4 mL, 7 M in MeOH) were added to a 25 mL round-bottomed flask. And the resulting mixture stirred for 16 h at 50° C. After this time, the mixture was concentrated to dryness in vacuo to give a yellow solid. The solid was then subjected to HPLC (Welch Xtimate C18 column, 5 μm, 150×30 mm 40-70% MeCN/H₂O with 0.05% NH₃H₂O+10 mM NH₄HCO₃) to give (R)-2-(3-(5-(3-hydroxy-1-methyl-2-oxopyrrolidin-3-yl)isoxazol-3-yl)phenyl)-5-isopropylthiazole-4-carboxamide as a colorless solid (42 mg, 32%). MS (ESI): Mass calcd. for C₂₁H₂₂N₄O₄S 426.14 m/z found 427.2 [M+H]⁺. ¹H NMR (400 MHz, DMSO-d₆) δ 8.48-8.45 (m, 1H), 8.14-8.10 (m, 1H), 8.02-7.98 (m, 2H), 7.68-7.58 (m, 2H), 7.19 (s, 1H), 6.76 (s, 1H), 4.39-4.29 (m, 1H), 3.53-3.41 (m, 2H), 2.85 (s, 3H), 2.63-2.54 (m, 1H), 2.37-2.23 (m, 1H), 1.35-1.29 (m, 6H).

### Example 220: (R)-2-(3-(5-(3-Hydroxy-1-methyl-2-oxopyrrolidin-3-yl)isoxazol-3-yl)phenyl)-5-isobutylthiazole-4-carboxamide

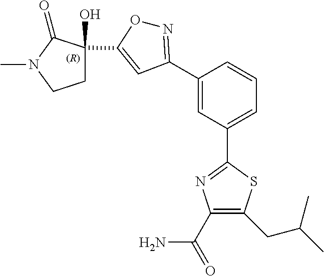

Step A: Methyl 2-bromo-5-isobutylthiazole-4-carboxylate. t-BuONO (1.69 mL, 14.1 mmol) was added to a solution consisting of methyl 2-amino-5-isobutylthiazole-4-carboxylate (1.00 g, 4.67 mmol) and CuBr₂ (2.06 g, 9.24 mmol) in CH₃CN (25 mL). The resulting mixture was heated at 50° C. for 2 h. After this time, the suspension was filtered and the filtrate concentrated to dryness in vacuo. The residue was subjected to silica gel chromatography (eluent: PE:EA:=1:0 to 3:1) to afford methyl 2-bromo-5-isobutylthiazole-4-carboxylate as a red oil (1.15 g, 86.0%). MS (ESI): Mass calcd. for C₉H₁₂BrNO₂S 276.98 m/z, found 277.6 [M+1]⁺.

Step B: (R)-Methyl 2-(3-(5-(3-hydroxy-1-methyl-2-oxopyrrolidin-3-yl)isoxazol-3-yl)phenyl)-5-isobutylthiazole-4-carboxylate. Methyl 2-bromo-5-isobutylthiazole-4-carboxylate (260 mg, 0.935 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 300 mg, 0.781 mmol), K₃PO₄ (418 mg, 1.97 mmol), Pd(dppf)Cl₂ (59 mg, 0.081 mmol), 1,4-dioxane (4 mL), and H₂O (1 mL) were added to a 40 mL vial. The resultant mixture was purged with N₂ for 5 min and then stirred for 12 h at 100° C. The reaction vessel was removed from the oil bath and allowed to gradually cool to rt. The mixture was diluted with H₂O (15 mL) and extracted with dichloromethane (15 mL×3). The combined organic extracts were washed with brine, dried over anhydrous Na₂SO₄, filtered, and concentrated to dryness in vacuo. The residue was subjected to silica gel chromatography (0-100% EtOAc/petroleum ether) to afford (R)-methyl 2-(3-(5-(3-hydroxy-1-methyl-2-oxopyrrolidin-3-yl)isoxazol-3-yl)phenyl)-5-isobutylthiazole-4-carboxylate as a yellow solid (220 mg, 52% yield). MS (ESI): Mass calcd. for C₂₃H₂₅N₃O₅S 455.15 m/z, found 456.1 [M+1]⁺.

Step C: (R)-2-(3-(5-(3-Hydroxy-1-methyl-2-oxopyrrolidin-3-yl)isoxazol-3-yl)phenyl)-5-isobutylthiazole-4-carboxamide. (R)-Methyl 2-(3-(5-(3-hydroxy-1-methyl-2-oxopyrrolidin-3-yl)isoxazol-3-yl)phenyl)-5-isobutylthiazole-4-carboxylate (220 mg, 0.483 mmol) was added into a solution of 7 M NH₃ in MeOH (21 mL, 147 mmol). The resultant mixture was stirred for 70 h at 50° C. After this time, the mixture was concentrated in vacuo. The residue was subjected to HPLC (Phenomenex Gemini-NX C18 column, 3 m, 75×30 mm; 36-66% (v/v) CH₃CN/H₂O with 0.05% NH₃H₂O+10 mM NH₄HCO₃) to give, after lyophilization, (R)-2-(3-(5-(3-hydroxy-1-methyl-2-oxopyrrolidin-3-yl)isoxazol-3-yl)phenyl)-5-isobutylthiazole-4-carboxamide as a white solid (93 mg, 42%). MS (ESI): Mass calcd. for C₂₂H₂₄N₄O₄S 440.15 m/z found 441.2 [M+H]⁺. ¹H NMR (400 MHz, DMSO-d₆) δ 8.51-8.44 (m, 1H), 8.14-8.07 (m, 1H), 8.04-7.96 (m, 2H), 7.69-7.61 (m, 1H), 7.57 (br s, 1H), 7.18 (s, 1H), 6.75 (s, 1H), 3.53-3.40 (m, 2H), 3.24 (d, J=7.2 Hz, 2H), 2.85 (s, 3H), 2.62-2.53 (m, 1H), 2.35-2.22 (m, 1H), 2.01-1.86 (m, 1H), 0.95 (d, J=6.8 Hz, 6H).

### Example 221: (R)-5-Cyclobutyl-2-(3-(5-(3-hydroxy-1-methyl-2-oxopyrrolidin-3-yl)isoxazol-3-yl)phenyl)thiazole-4-carboxamide

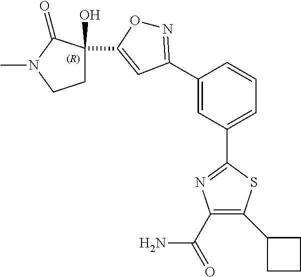

Step A: Methyl 2-amino-5-cyclobutylthiazole-4-carboxylate. A solution of NaOMe (2.79 g, 51.6 mmol) in MeOH (40 mL) was added to a mixture consisting of methyl 2,2-dichloroacetate (5.33 mL, 47.7 mmol) and cyclobutanecarbaldehyde (3.90 g, 46.4 mmol) in THF (40 mL) dropwise at 0° C. The resultant mixture was stirred at 0° C. for 1 hour. After this time, the reaction mixture was quenched with H₂O (120 mL) and extracted with ethyl acetate (80 mL×2). The combined organic extracts were washed with brine, dried over anhydrous Na₂SO₄, filtered, and concentrated to dryness in vacuo to afford a colorless oil. Thiourea (3.51 g, 46.1 mmol) and the colorless oil were added to MeOH (40 mL) and the resulting mixture was refluxed for 16 h. After this time, the mixture was concentrated to dryness in vacuo, and the residue subjected to silica gel chromatography (0-100% EtOAc/petroleum ether) to afford methyl 2-amino-5-cyclobutylthiazole-4-carboxylate as a yellow solid (400 mg, 4%). MS (ESI): Mass calcd. for C₉H₁₂N₂O₂S 212.06 m/z, found 213.0 [M+1]⁺.

Step B: Methyl 2-bromo-5-cyclobutylthiazole-4-carboxylate. t-BuONO (0.85 mL, 7.12 mmol) was added to a solution consisting of methyl 2-amino-5-cyclobutylthiazole-4-carboxylate (500 mg, 2.36 mmol), CuBr₂ (1.04 g, 4.66 mmol), and CH₃CN (10 mL). The resultant mixture was stirred for 2 h at 50° C. After this time, the suspension was filtered and the filtrate concentrated to dryness in vacuo. The residue was subjected to silica gel chromatography (0-25% EtOAc/petroleum ether) to afford methyl 2-bromo-5-cyclobutylthiazole-4-carboxylate (440 mg, 67.6%) as a yellow oil. MS (ESI): Mass calcd. for C₉H₁₀BrNO₂S 274.96 m/z, found 275.7 [M+1]⁺.

Step C: (R)-Methyl 5-cyclobutyl-2-(3-(5-(3-hydroxy-1-methyl-2-oxopyrrolidin-3-yl)isoxazol-3-yl)phenyl)thiazole-4-carboxylate. Methyl 2-bromo-5-cyclobutylthiazole-4-carboxylate (260 mg, 0.942 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 300 mg, 0.781 mmol), K₃PO₄ (418 mg, 1.97 mmol), Pd(dppf)Cl₂ (59 mg, 0.081 mmol), 1,4-dioxane (4 mL), and H₂O (1 mL) were added to a 10 mL flask, and the resulting mixture purged with N₂ for 5 min and then stirred for 12 h at 100° C. The reaction vessel was removed from the oil bath and allowed to gradually cool to RT. The reaction mixture was diluted with H₂O (15 mL) and extracted with dichloromethane (15 mL×3). The combined organic extracts were washed with brine (10 mL×2), dried over anhydrous Na₂SO₄, filtered, and concentrated to dryness in vacuo. The residue was subjected to silica gel chromatography (0-100% EtOAc/petroleum ether) to afford (R)-methyl 5-cyclobutyl-2-(3-(5-(3-hydroxy-1-methyl-2-oxopyrrolidin-3-yl)isoxazol-3-yl)phenyl)thiazole-4-carboxylate as a yellow oil (260 mg, 59.1% yield). MS (ESI): Mass calcd. for C₂₃H₂₃N₃O₅S 453.14 m/z, found 454.0 [M+1]⁺.

Step D: (R)-5-Cyclobutyl-2-(3-(5-(3-hydroxy-1-methyl-2-oxopyrrolidin-3-yl)isoxazol-3-yl)phenyl)thiazole-4-carboxamide. (R)-Methyl 5-cyclobutyl-2-(3-(5-(3-hydroxy-1-methyl-2-oxopyrrolidin-3-yl)isoxazol-3-yl)phenyl)thiazole-4-carboxylate (260 mg, 0.573 mmol) was added to a solution of 7 M NH₃ in MeOH (25 mL, 175 mmol), and the resulting mixture was stirred for 70 h at 50° C. After this time, the mixture was concentrated in vacuo. The residue was subjected to HPLC (Phenomenex Gemini-NX C18 column, 3 m, 75×30 mm (35-65% CH₃CN/H₂O with 0.05% NH₃H₂O+10 mM NH₄HCO₃)) to give, after lyophilization, (R)-5-cyclobutyl-2-(3-(5-(3-hydroxy-1-methyl-2-oxopyrrolidin-3-yl)isoxazol-3-yl)phenyl)thiazole-4-carboxamide as a white solid (103.3 mg, 40.8%). MS (ESI): Mass calcd. for C₂₂H₂₂N₄O₄S 438.14 m/z, found 439.2 [M+H]⁺. ¹H NMR (400 MHz, DMSO-d₆) δ 8.50-8.45 (m, 1H), 8.15-8.09 (m, 1H), 8.04-7.98 (m, 1H), 7.94 (br s, 1H), 7.70-7.61 (m, 1H), 7.57 (br s, 1H), 7.19 (s, 1H), 6.75 (s, 1H), 4.74-4.61 (m, 1H), 3.54-3.39 (m, 2H), 2.85 (s, 3H), 2.63-2.52 (m, 3H), 2.35-2.24 (m, 1H), 2.14-1.95 (m, 3H), 1.94-1.83 (m, 1H).

### Example 222: (R)-5-(Difluoromethyl)-2-(3-(5-(3-hydroxy-1-methyl-2-oxopyrrolidin-3-yl)isoxazol-3-yl)phenyl)thiazole-4-carboxamide

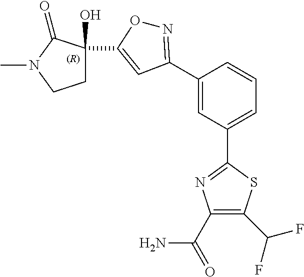

Step A: Methyl 2-bromo-5-(dibromomethyl)thiazole-4-carboxylate. To a mixture consisting of methyl 2-bromo-5-methylthiazole-4-carboxylate (500 mg, 2.12 mmol), N-bromosuccinimide (1.36 g, 6.35 mmol), and carbon tetrachloride (20 mL) in a 40 mL vial was added benzoyl peroxide (103 mg, 0.42 mmol) in one portion. The resulting mixture was stirred for 3 h at 80° C. After this time, the reaction vessel was removed from the oil bath and allowed to gradually cool to rt. The orange mixture was combined with 3 other batches of reaction mixture and concentrated in vacuo to give an orange solid, which was subjected to silica gel chromatography (0-10% EtOAc/petroleum ether) to afford methyl 2-bromo-5-(dibromomethyl)thiazole-4-carboxylate (3.0 g, 90%) as a pale yellow oil. MS (ESI): Mass calcd. for C₆H₄Br₃NO₂S 390.75 m/z, found 391.8 [M+H]⁺.

Step B Methyl 2-bromo-5-formylthiazole-4-carboxylate. Methyl 2-bromo-5-(dibromomethyl)thiazole-4-carboxylate (500 mg, 1.27 mmol), silver nitrate (524 mg, 5.08 mmol), ethanol (10 mL), and water (1 mL) were added to a 40 mL vial. The resulting mixture was stirred for 3 h at 80° C. After this time, the reaction vessel was removed from the heating device and allowed to gradually cool to rt. The mixture was combined with the other 3 batches, filtered through a pad of diatomaceous earth and the pad washed with EtOAc (50 mL). The filtrate was diluted with EtOAc (100 mL) and washed with water (20 mL×3) and brine (20 mL×3), dried over anhydrous Na₂SO₄, and filtered. The filtrate was concentrated in vacuo to give a white solid, which was subjected to silica gel chromatography (0-10% EtOAc/petroleum ether) to give methyl 2-bromo-5-formylthiazole-4-carboxylate (1.0 g, 79%) as a white solid. ¹H NMR (400 MHz, CDCl₃) δ 10.61 (s, 1H), 4.07 (s, 3H).

Step C: Methyl 2-bromo-5-(difluoromethyl)thiazole-4-carboxylate. Methyl 2-bromo-5-formylthiazole-4-carboxylate (750 mg, 3.00 mmol) and CH₂Cl₂ (30 mL) were added to a 100 mL three-necked round-bottomed flask. The resulting mixture was cooled with an ice/water bath, treated with DAST (1.94 mL, 12.0 mmol, in 2.5 mL of anhydrous CH₂Cl₂) dropwise over a course of 10 min, and stirred for 2 h at 0° C. After this time, the reaction was quenched with saturated aqueous NaHCO₃ (25 mL) at 0° C. and extracted with CH₂Cl₂ (25 mL×3). The combined organic extracts were dried over anhydrous Na₂SO₄, filtered, and the filtrate concentrated in vacuo to give a pale yellow gum, which was subjected to silica gel chromatography (0-10% EtOAc/petroleum ether) to give methyl 2-bromo-5-(difluoromethyl)thiazole-4-carboxylate as a colorless oil (600 mg, 74%).

Step D: 2-Bromo-5-(difluoromethyl)thiazole-4-carboxamide. Methyl 2-bromo-5-(difluoromethyl)thiazole-4-carboxylate (500 mg, 1.84 mmol), and MeOH (5 mL) were added to a 40 mL vial. NH₃ (2.6 mL, 18 mmol, 7 M in MeOH) was added in one portion. The vial was capped and stirred for 4 h at room temperature. The mixture was concentrated in vacuo to give a white solid, which was subjected to silica gel chromatography (0-50% EtOAc/petroleum ether) to afford 2-bromo-5-(difluoromethyl)thiazole-4-carboxamide as a white solid (380 mg, 80%).

Step E: (R)-5-(Difluoromethyl)-2-(3-(5-(3-hydroxy-1-methyl-2-oxopyrrolidin-3-yl)isoxazol-3-yl)phenyl)thiazole-4-carboxamide. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 314 mg, 0.817 mmol), 2-bromo-5-(difluoromethyl)thiazole-4-carboxamide (300 mg, 1.17 mmol), NaHCO₃ (196 mg, 2.33 mmol), Pd(dtbpf)Cl₂ (76 mg, 0.12 mmol), H₂O (1 mL), and 1,4-dioxane (10 mL) were added to a 40 mL vial. The vial was subsequently evacuated and refilled with N₂ 3 times. The vial was capped and heated at 80° C. for 2 h. After this time, the reaction vessel was removed from the heating device and allowed to gradually cool to rt. The mixture was filtered through a pad of diatomaceous earth, and the pad washed with MeOH (10 mL). The filtrate was concentrated to dryness in vacuo to give a brown gum. The gum was subjected to HPLC (Boston Green ODS column, 5 μm, 150×30 mm; 25-55% CH₃CN/H₂O with 0.225% FA), and then subjected to SFC (DAICEL CHIRALCEL® AS column, 10 μm, 250×30 mm; 70% EtOH (containing 0.1% NH₃H₂O)/supercritical CO₂) to give, after lyophilization, (R)-5-(difluoromethyl)-2-(3-(5-(3-hydroxy-1-methyl-2-oxopyrrolidin-3-yl)isoxazol-3-yl)phenyl)thiazole-4-carboxamide as a white solid (65.3 mg, 13%). MS (ESI): Mass calcd. for C₁₉H₁₆F₂N₄O₄S 434.09 m/z, found 435.1 [M+H]⁺. ¹H NMR (400 MHz, DMSO-d₆) δ 8.59 (s, 1H), 8.37 (s, 1H), 8.23 (d, J=8.0 Hz, 1H), 8.13-7.77 (m, 3H), 7.75-7.66 (m, 1H), 7.22 (s, 1H), 6.78 (s, 1H), 3.54-3.41 (m, 2H), 2.85 (s, 3H), 2.62-2.54 (m, 1H), 2.34-2.24 (m, 1H). ¹H NMR (400 MHz, CD₃OD) δ 8.54 (s, 1H), 8.14 (d, J=8.0 Hz, 1H), 8.05-7.71 (m, 2H), 7.68-7.61 (m, 1H), 7.04 (s, 1H), 3.66-3.55 (m, 2H), 2.99 (s, 3H), 2.83-2.70 (m, 1H), 2.48-2.38 (m, 1H).

### Example 223: (R)-5-((Cyclopentylamino)methyl)-2-(3-(5-(3-hydroxy-1-methyl-2-oxopyrrolidin-3-yl)isoxazol-3-yl)phenyl)thiazole-4-carboxamide

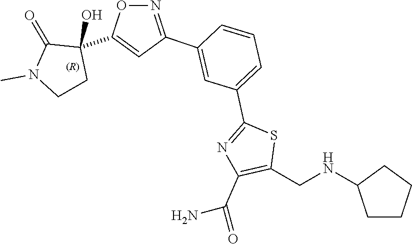

Step A: Methyl 2-bromo-5-(bromomethyl)thiazole-4-carboxylate. Methyl 2-bromo-5-methylthiazole-4-carboxylate (100 mg, 0.424 mmol), N-bromosuccinimide (75.4 mg, 0.424 mmol), benzoyl peroxide (20.5 mg, 0.085 mmol), and carbon tetrachloride (4 mL) were added to an 8 mL vial. The resulting mixture was stirred for 3 h at 80° C. The reaction vessel was removed from the heating device and allowed to gradually cool to rt. The reaction mixture was concentrated in vacuo to give an orange solid, which was then subjected to silica gel chromatography (0-10% EtOAc/petroleum ether) to afford methyl 2-bromo-5-(bromomethyl)thiazole-4-carboxylate as a pale yellow oil (70 mg, 52%). ¹H NMR (400 MHz, CDCl₃) δ 5.12-5.00 (m, 2H), 4.03-3.96 (m, 3H).

Step B: Methyl 2-bromo-5-((cyclopentylamino)methyl)thiazole-4-carboxylate. Methyl 2-bromo-5-(bromomethyl)thiazole-4-carboxylate (200 mg, 0.635 mmol), cyclopentanamine (54.1 mg, 0.635 mmol), DIPEA (0.221 mL, 1.27 mmol), and CH₂Cl₂ (3 mL) were added to a 8 mL vial, and the resulting mixture stirred for 16 h at rt. After this time, the mixture was concentrated in vacuo to give a orange solid, which was then subjected to silica gel chromatography (0-20% EtOAc/petroleum ether) to afford methyl 2-bromo-5-((cyclopentylamino)methyl)thiazole-4-carboxylate as a yellow oil (100 mg, 49%). ¹H NMR (400 MHz, DMSO-d₆) δ 4.10 (s, 2H), 3.81 (s, 3H), 3.13-3.06 (m, 1H), 2.93 (s, 1H), 1.77-1.67 (m, 2H), 1.66-1.58 (m, 2H), 1.51-1.43 (m, 2H), 1.36-1.25 (m, 2H).

Step C: (R)-Methyl 5-((cyclopentylamino)methyl)-2-(3-(5-(3-hydroxy-1-methyl-2-oxopyrrolidin-3-yl)isoxazol-3-yl)phenyl)thiazole-4-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 102 mg, 0.265 mmol), methyl 2-bromo-5-((cyclopentylamino)methyl)thiazole-4-carboxylate (84.7 mg, 0.265 mmol), Pd(dtbpf)Cl₂ (17.3 mg, 0.027 mmol), K₃PO₄ (113 mg, 0.531 mmol), 1,4-dioxane (3.00 mL), and H₂O (0.3 mL) were added to an 8 mL vial, which was subsequently evacuated and refilled with Ar (×3), and then stirred for 3 h at 85° C. The reaction vessel was removed from the heating device and allowed to gradually cool to room temperature. The reaction mixture was then concentrated in vacuo to give a brown oil, which was subjected to silica gel chromatography (0-20% MeOH/CH₂Cl₂) to afford (R)-methyl 5-((cyclopentylamino)methyl)-2-(3-(5-(3-hydroxy-1-methyl-2-oxopyrrolidin-3-yl)isoxazol-3-yl)phenyl)thiazole-4-carboxylate as a dark yellow oil (200 mg, 98%). MS (ESI): Mass calcd. for C₂₅H₂₈N₄O₅S 496.18 m/z, found 497.3 [M+H]⁺.

Step D: (R)-5-((Cyclopentylamino)methyl)-2-(3-(5-(3-hydroxy-1-methyl-2-oxopyrrolidin-3-yl)isoxazol-3-yl)phenyl)thiazole-4-carboxamide. (R)-Methyl 5-((cyclopentylamino)methyl)-2-(3-(5-(3-hydroxy-1-methyl-2-oxopyrrolidin-3-yl)isoxazol-3-yl)phenyl)thiazole-4-carboxylate (190 mg, 0.383 mmol) and NH₃ (15 mL, 0.105 mol, 7 M in MeOH) were added to a 40 mL vial and the resulting mixture heated to 50° C. and stirred for 16 h. The reaction vessel was removed from the oil bath and allowed to gradually cool to rt. The mixture was concentrated to dryness in vacuo, and the residue subjected to HPLC (Welch Xtimate C18 column, 5 μm, 150×30 mm; 30-60% MeCN/water with 0.05% NH₃H₂O+10 mM NH₄HCO₃) to give, after lyophilization, (R)-5-((cyclopentylamino)methyl)-2-(3-(5-(3-hydroxy-1-methyl-2-oxopyrrolidin-3-yl)isoxazol-3-yl)phenyl)thiazole-4-carboxamide as yellow solid (52.3 mg, 25%). MS (ESI): Mass calcd. for C₂₄H₂₇N₅O₄S 481.18 m/z, found 482.3 [M+H]⁺. ¹H NMR (400 MHz, DMSO-d₆) δ 8.46 (s, 1H), 8.11 (d, J=7.6 Hz, 1H), 8.02-7.94 (m, 2H), 7.68-7.61 (m, 1H), 7.58 (s, 1H), 7.17 (s, 1H), 6.75 (s, 1H), 4.24 (s, 2H), 3.52-3.46 (m, 2H), 3.18-3.05 (m, 1H), 2.85 (s, 3H), 2.63-2.53 (m, 2H), 2.35-2.21 (m, 1H), 1.80-1.69 (m, 2H), 1.68-1.59 (m, 2H), 1.53-1.42 (m, 2H), 1.41-1.29 (m, 2H). ¹H NMR (400 MHz, MeOH-d₄) δ 8.50 (s, 1H), 8.10 (d, J=7.6 Hz, 1H), 7.97 (d, J=7.6 Hz, 1H), 7.66-7.60 (m, 1H), 7.03 (s, 1H), 4.32 (s, 2H), 3.65-3.55 (m, 2H), 3.23-3.14 (m, 1H), 2.99 (s, 3H), 2.82-2.72 (m, 1H), 2.48-2.38 (m, 1H), 1.98-1.86 (m, 2H), 1.82-1.70 (m, 2H), 1.66-1.54 (m, 2H), 1.51-1.40 (m, 2H).

### Example 224: (R)-3-Hydroxy-3-(3-(3-(4-methoxy-1H-pyrazolo[3,4-b]pyridin-6-yl)phenyl)isoxazol-5-yl)-1-methylpyrrolidin-2-one

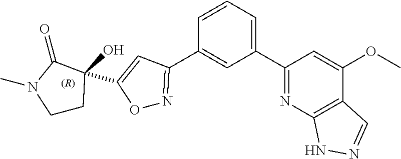

Step A: Ethyl 5-amino-1-(4-methoxybenzyl)-1H-pyrazole-4-carboxylate. (4-Methoxybenzyl) hydrazine (4.9 g, 33 mmol) was added to a solution consisting of (E)-ethyl 2-cyano-3-ethoxyacrylate (5.0 g, 30 mmol) and anhydrous EtOH (70 mL). The resulting mixture was heated at 90° C. for 16 h. The reaction vessel was removed from the oil bath and allowed to gradually cool to rt. The reaction mixture was filtered through a pad of diatomaceous earth. The filtrate was diluted with CH₂Cl₂ (30 mL), washed with H₂O (30 mL) and brine (20 mL), dried over anhydrous Na₂SO₄, filtered, and concentrated to dryness in vacuo. The residue was subjected to silica-gel chromatography (0-30% EtOAc/petroleum ether) to afford ethyl 5-amino-1-(4-methoxybenzyl)-1H-pyrazole-4-carboxylate (1.8 g, 22%) as a white solid. MS (ESI): Mass calcd. for C₁₄H₁₇N₃O₃ 275.12, m/z found 276.2 [M+H]⁺.

Step B: Ethyl 1-(4-methoxybenzyl)-4,6-dioxo-4,5,6,7-tetrahydro-1H-pyrazolo[3,4-b]pyridine-5-carboxylate. Diethyl malonate (0.873 g, 5.45 mmol) was dissolved in anhydrous EtOH (8 mL), then cooled to 0° C. (ice water bath). Then, a 20% solution of NaOEt (1.85 g, 5.45 mmol) in EtOH was added dropwise (within 10 min) at 0° C. The cooling bath was removed and the reaction was stirred at room temperature for 15 min, treated with ethyl 5-amino-1-(4-methoxybenzyl)-1H-pyrazole-4-carboxylate (300 mg, 1.09 mmol) in portions (within 2 min), and heated at 90° C. under reflux condensation for 3 days. The reaction was cooled to room temperature and concentrated to dryness in vacuo. The residue was combined with 7 other batches of this reaction, suspended in CH₂Cl₂ (200 mL) and extracted with H₂O (100 mL×3). The combined aqueous phases were concentrated to dryness in vacuo to afford ethyl 1-(4-methoxybenzyl)-4,6-dioxo-4,5,6,7-tetrahydro-1H-pyrazolo[3,4-b]pyridine-5-carboxylate (10 g) as a yellow solid. MS (ESI): Mass calcd. for C₁₇H₁₇N₃O₅ 343.1 m/z, found 344.1 [M+H]⁺.

Step C: 1-(4-Methoxybenzyl)-1H-pyrazolo[3,4-b]pyridine-4,6-diol. Sodium hydroxide (7.5 g, 188 mmol) was slowly added to H₂O (42 mL) within 10 minutes. After the previous mixture had cooled to room temperature, the sodium hydroxide mixture and ethyl 1-(4-methoxybenzyl)-4,6-dioxo-4,5,6,7-tetrahydro-1H-pyrazolo[3,4-b]pyridine-5-carboxylate (5 g, 15 mmol) were added to a 250 mL three-neck round-bottomed flask equipped with a condenser, and a thermometer. The resulting mixture was heated at 100° C. for 16 h. The reaction vessel was removed from the oil bath and allowed to gradually cool to rt. The reaction mixture was then neutralized to pH 6-7 with 2 M aqueous HCl and filtered. The filter cake was dried in vacuo to afford 1-(4-methoxybenzyl)-1H-pyrazolo[3,4-b]pyridine-4,6-diol (1 g, 25%) as a pink solid. MS (ESI): Mass calcd. for C₁₄H₁₃N₃O₃ 271.09, m/z found 272.1 [M+H]⁺.

Step D: 4,6-Dichloro-1-(4-methoxybenzyl)-1H-pyrazolo[3,4-b]pyridine. A mixture consisting of 1-(4-methoxybenzyl)-1H-pyrazolo[3,4-b]pyridine-4,6-diol (1.00 g, 3.69 mmol) and phenylphosphonic dichloride (5.0 mL) was heated at 150° C. for 3 h. The reaction vessel was removed from the oil bath and allowed to gradually cool to rt. The reaction mixture was then poured into ice water (200 mL), neutralized with 28% NH₄OH to pH 7-8, and extracted with DCM (40 mL×3). The combined extracts were washed with brine (20 mL), dried with anhydrous Na₂SO₄, filtered, and concentrated in vacuo to afford a residue, which was subjected to silica gel chromatography (0-20% EtOAc/petroleum ether) to afford 4,6-dichloro-1-(4-methoxybenzyl)-1H-pyrazolo[3,4-b]pyridine (348 mg, 30%) as a white solid. MS (ESI): Mass calcd. for C₁₄H₁₁Cl₂N₃O 307.02 m/z, found 308.0 [M+H]⁺.

Step E: 6-Chloro-4-methoxy-1-(4-methoxybenzyl)-1H-pyrazolo[3,4-b]pyridine. To a solution consisting of 4,6-dichloro-1-(4-methoxybenzyl)-1H-pyrazolo[3,4-b]pyridine (348 mg, 1.13 mmol) in anhydrous THF (5.0 mL) was added NaOMe (0.90 mL, 0.90 mmol, 1.0 M in THF) dropwise within 2 min. The resultant mixture was stirred for 2 h at 60° C. The reaction vessel was removed from the oil bath and allowed to gradually cool to rt. The reaction mixture was then purified by silica gel chromatography (0-10% EtOAc/petroleum ether) to afford 6-chloro-4-methoxy-1-(4-methoxybenzyl)-1H-pyrazolo[3,4-b]pyridine as a white solid (215 mg, 62%). ¹H NMR (400 MHz, CDCl₃) δ 7.99 (s, 1H), 7.32 (d, J=8.6 Hz, 2H), 6.84 (d, J=8.6 Hz, 2H), 6.53 (s, 1H), 5.55 (s, 2H), 4.02 (s, 3H), 3.77 (s, 3H).

Step F: (R)-3-Hydroxy-3-(3-(3-(4-methoxy-1-(4-methoxybenzyl)-1H-pyrazolo[3,4-b]pyridin-6-yl)phenyl)isoxazol-5-yl)-1-methylpyrrolidin-2-one. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 190 mg, 0.026 mmol), 6-chloro-4-methoxy-1-(4-methoxybenzyl)-1H-pyrazolo[3,4-b]pyridine (150 mg, 0.49 mmol), K₃PO₄ (336 mg, 1.48 mmol), 1,4-dioxane (8 mL), and water (0.8 mL) were added to a 20 mL microwave tube, which was subsequently evacuated and refilled with argon (×3). The mixture was treated with Pd(dtbpf)Cl₂ (32 mg, 0.049 mmol), and heated at 100° C. for 2 h under N₂. The reaction vessel was removed from oil bath and allowed to gradually cool to rt. The mixture was then filtered through a pad of diatomaceous earth, and the pad washed with EtOAc (15 mL). The filtrate was concentrated to dryness in vacuo. The residue was subjected to silica gel chromatography (0-100% EtOAc/petroleum ether) to afford (R)-3-hydroxy-3-(3-(3-(4-methoxy-1-(4-methoxybenzyl)-1H-pyrazolo[3,4-b]pyridin-6-yl)phenyl)isoxazol-5-yl)-1-methylpyrrolidin-2-one as a pale yellow gum (200 mg, 77%). MS (ESI): Mass calcd. for C₂₉H₂₇N₅O₅ 525.20, m/z found 526.2 [M+H]⁺.

Step G: (R)-3-Hydroxy-3-(3-(3-(4-methoxy-1H-pyrazolo[3,4-b]pyridin-6-yl)phenyl)isoxazol-5-yl)-1-methylpyrrolidin-2-one. A solution consisting of (R)-3-hydroxy-3-(3-(3-(4-methoxy-1-(4-methoxybenzyl)-1H-pyrazolo[3,4-b]pyridin-6-yl)phenyl)isoxazol-5-yl)-1-methylpyrrolidin-2-one (200 mg, 0.38 mmol) and TFA (10 mL) was stirred at room temperature for 20 h. After this time, the mixture was concentrated in vacuo. The residue was subjected to HPLC with a Boston Green ODS 150×30 mm×5 m column (eluent: 25-55% (v/v) CH₃CN and H₂O with 0.225% FA) to give, after lyophillization, (R)-3-hydroxy-3-(3-(3-(4-methoxy-1H-pyrazolo[3,4-b]pyridin-6-yl)phenyl)isoxazol-5-yl)-1-methylpyrrolidin-2-one (73.7 mg, 48.0%) as a white solid. MS (ESI): Mass calcd. for C₂₁H₁₉N₅O₄ 405.4, m/z found 406.2 [M+H]⁺. ¹H NMR (400 MHz, DMSO-d₆) δ 13.66 (s, 1H), 8.64 (s, 1H), 8.31 (d, J=8.0 Hz, 1H), 8.15 (s, 1H), 7.98 (d, J=7.6 Hz, 1H), 7.72-7.63 (m, 1H), 7.34 (s, 1H), 7.12 (s, 1H), 6.77 (s, 1H), 4.17 (s, 3H), 3.53-3.45 (m, 2H), 2.86 (s, 3H), 2.63-2.55 (m, 1H), 2.35-2.25 (m, 1H). ¹H NMR (400 MHz, CD₃OD) δ 8.60-8.54 (m, 1H), 8.22 (d, J=8.4 Hz, 1H), 8.11 (s, 1H), 7.94 (d, J=8.0 Hz, 1H), 7.68-7.60 (m, 1H), 7.21 (s, 1H), 7.02 (s, 1H), 4.19 (s, 3H), 3.63-3.57 (m, 2H), 2.99 (s, 3H), 2.81-2.72 (m, 1H), 2.48-2.38 (m, 1H).

### Example 225: (R)-3-Hydroxy-1-methyl-3-(3-(3-(4-(trifluoromethyl)-1H-pyrazolo[3,4-b]pyridin-6-yl)phenyl)isoxazol-5-yl)pyrrolidin-2-one

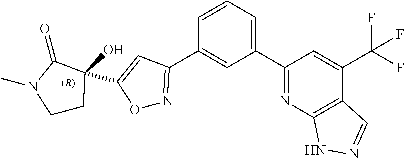

Step A: 4-Iodo-1H-pyrazolo[3,4-b]pyridine 7-oxide. A solution consisting of 4-iodo-1H-pyrazolo[3,4-b]pyridine (750 mg, 3.1 mmol) and CH₂Cl₂ (15 mL) was added m-CPBA (1.24 g, 6.1 mmol, 85% purity) in portions over 2 min. The mixture was stirred for 18 h at room temperature. After this time, the mixture was diluted with 25 mL of MTBE and filtered. The filter cake was washed with MTBE (15 mL) and concentrated to dryness in vacuo to afford 4-iodo-1H-pyrazolo[3,4-b]pyridine 7-oxide as a pale yellow solid (600 mg, 75%). MS (ESI): Mass calcd. for C₆H₄₁N₃O 261.02, m/z found 262.0 [M+H]⁺.

Step B: 6-Chloro-4-iodo-1H-pyrazolo[3,4-b]pyridine. To a mixture consisting of 4-iodo-1H-pyrazolo[3,4-b]pyridine 7-oxide (600 mg, 2.3 mmol) and MeCN (5 mL) was added POCl_{3 (}1.5 mL) in portions over 2 min. The resultant mixture was stirred for 2 h at room temperature. After this time, the mixture was neutralized with 1 N aqueous NaOH and filtered. The filter cake was washed with water and concentrated to dryness in vacuo to afford the title compound, 6-chloro-4-iodo-1H-pyrazolo[3,4-b]pyridine, as a pale yellow solid (600 mg, 93%). MS (ESI): Mass calcd. for C₆H₃C₁₁N₃ 278.90, m/z found 279.9 [M+H]⁺.

Step C: 6-Chloro-4-iodo-1-(tetrahydro-2H-pyran-2-yl)-1H-pyrazolo[3,4-b]pyridine. A mixture consisting of 6-chloro-4-iodo-1H-pyrazolo[3,4-b]pyridine (300 mg, 1.07 mmol), pyridinium p-toluenesulfonate (45 mg, 0.11 mmol), 3,4-dihydro-2H-pyran (0.29 mL, 3.2 mmol), and CH₂Cl₂ (5.0 mL) was refluxed for 18 h under a N₂ atmosphere. The reaction vessel was removed from the oil bath and allowed to gradually cool to RT. The reaction mixture was concentrated to dryness in vacuo. The residue was purified by silica gel chromatography (0-20% EtOAc/petroleum ether) to afford 6-chloro-4-iodo-1-(tetrahydro-2H-pyran-2-yl)-1H-pyrazolo[3,4-b]pyridine as a pale yellow solid (180 mg, 46%). MS (ESI): Mass calcd. for C₁₁H₁₁C₁₁N₃O 362.96, m/z found 364.0 [M+H]⁺.

Step D: 6-Chloro-1-(tetrahydro-2H-pyran-2-yl)-4-(trifluoromethyl)-1H-pyrazolo[3,4-b]pyridine. 6-Chloro-4-iodo-1-(tetrahydro-2H-pyran-2-yl)-1H-pyrazolo[3,4-b]pyridine (120 mg, 0.33 mmol), methyl 2,2-difluoro-2-(fluorosulfonyl)acetate (159 mg, 0.83 mmol), CuI (157 mg, 0.83 mmol), and DMF (2.5 mL) were added to an 8 mL vial.

The resulting mixture was purged with N₂ for 5 minutes, and then heated at 100° C. for 20 h. The reaction vessel was removed from oil bath and allowed to gradually cool to room temperature. The reaction mixture was then filtered through a pad of diatomaceous earth, and the pad washed with EtOAc (15 mL). The filtrate was concentrated in vacuo. The residue was purified by silica gel chromatography (0-20% EtOAc/petroleum ether) to afford 6-chloro-1-(tetrahydro-2H-pyran-2-yl)-4-(trifluoromethyl)-1H-pyrazolo[3,4-b]pyridine as a pale yellow solid (50 mg, 50%). MS (ESI): Mass calcd. for Cl₂H₁₁ClF₃N₃O 305.05, m/z found 222.1 [M-THP]⁺.

Step E: (R)-3-Hydroxy-1-methyl-3-(3-(3-(1-(tetrahydro-2H-pyran-2-yl)-4-(trifluoromethyl)-1H-pyrazolo[3,4-b]pyridin-6-yl)phenyl)isoxazol-5-yl)pyrrolidin-2-one. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 63 mg, 0.16 mmol), 6-chloro-1-(tetrahydro-2H-pyran-2-yl)-4-(trifluoromethyl)-1H-pyrazolo[3,4-b]pyridine (50 mg, 0.16 mmol), K₃PO₄ (67 mg, 0.49 mmol), 1,4-dioxane (5 mL), and water (0.5 mL) was sparged with N₂ for 5 minutes, treated with Pd(dtbpf)Cl₂ (16 mg, 0.025 mmol), and heated at 100° C. for 2 h under a N₂ atmosphere. The reaction vessel was removed from the oil bath and allowed to gradually cool to room temperature. The reaction mixture was filtered through a pad of diatomaceous earth and the pad washed with EtOAc (15 mL). The filtrate was concentrated in vacuo. The residue was purified by silica gel chromatography (0-100% EtOAc/petroleum ether) to afford (R)-3-hydroxy-1-methyl-3-(3-(3-(1-(tetrahydro-2H-pyran-2-yl)-4-(trifluoromethyl)-1H-pyrazolo[3,4-b]pyridin-6-yl)phenyl)isoxazol-5-yl)pyrrolidin-2-one (65 mg, 77%) as a pale yellow solid. MS (ESI): Mass calcd. for C₂₆H₂₄F₃N₅O₄ 527.17, m/z found 528.2 [M+H]⁺.

Step F: (R)-3-Hydroxy-1-methyl-3-(3-(3-(4-(trifluoromethyl)-1H-pyrazolo[3,4-b]pyridin-6-yl)phenyl)isoxazol-5-yl)pyrrolidin-2-one. To a solution consisting of (R)-3-hydroxy-1-methyl-3-(3-(3-(1-(tetrahydro-2H-pyran-2-yl)-4-(trifluoromethyl)-1H-pyrazolo[3,4-b]pyridin-6-yl)phenyl)isoxazol-5-yl)pyrrolidin-2-one (60 mg, 0.114 mmol) and MeOH (2.5 mL) was added HCl (2.5 mL, 2.0 mmol, 4 M in 1,4-dioxane) in one portion. The resultant mixture was stirred for 2 h at room temperature. After this time, the mixture was concentrated in vacuo. The residue was subjected to HPLC (Boston Green ODS column, 5 μm, 150×30 mm; 40-70% (v/v) CH₃CN/H₂O with 0.225% FA) to give, after lyophillization, (R)-3-hydroxy-1-methyl-3-(3-(3-(4-(trifluoromethyl)-1H-pyrazolo[3,4-b]pyridin-6-yl)phenyl)isoxazol-5-yl)pyrrolidin-2-one as a white solid (16.6 mg, 33%). MS (ESI): Mass calcd. for C₂₁H₁₆F₃N₅O₃ 443.4 m/z, found 444.1 [M+H]⁺. 1H NMR (400 MHz, DMSO-d₆) δ 14.38 (br s, 1H), 8.72 (s, 1H), 8.41 (d, J=8.0 Hz, 1H), 8.35 (d, J=1.2 Hz, 1H), 8.32 (s, 1H), 8.05 (d, J=8.0 Hz, 1H), 7.76-7.68 (m, 1H), 7.20 (s, 1H), 6.79 (s, 1H), 3.54-3.46 (m, 2H), 2.86 (s, 3H), 2.64-2.55 (m, 1H), 2.35-2.24 (m, 1H). ¹H NMR (400 MHz, CDCl₃) δ 12.17 (br s, 1H), 8.40 (s, 1H), 8.23 (d, J=1.2 Hz, 1H), 8.04 (d, J=8.0 Hz, 1H), 7.82-7.75 (m, 2H), 7.56-7.47 (m, 1H), 6.85 (s, 1H), 5.30 (br s, 1H), 3.71-3.62 (m, 1H), 3.59-3.49 (m, 1H), 3.03 (s, 3H), 2.85-2.76 (m, 1H), 2.57-2.46 (m, 1H).

### Example 226: (R)-3-(3-(3-(1H-Pyrazolo[3,4-d]pyrimidin-6-yl)phenyl)isoxazol-5-yl)-3-hydroxy-1-methylpyrrolidin-2-one

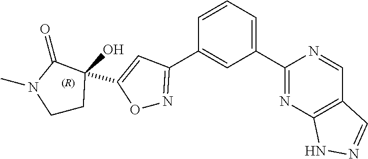

Step A: 6-Chloro-1-(tetrahydro-2H-pyran-2-yl)-1H-pyrazolo[3,4-d]pyrimidine. A mixture consisting of 6-chloro-1H-pyrazolo[3,4-d]pyrimidine (1.0 g, 6.5 mmol), pyridinium p-toluenesulfonate (271 mg, 0.65 mmol), 3,4-dihydro-2H-pyran (1.2 mL, 12.9 mmol), and CH₂Cl₂ (15.0 mL) was refluxed for 18 h under a N₂ atmosphere. The reaction vessel was removed from the oil bath and allowed to gradually cool to rt. The mixture was then concentrated to dryness in vacuo and the residue subjected to silica gel chromatography (0-40% EtOAc/petroleum ether) to give 6-chloro-1-(tetrahydro-2H-pyran-2-yl)-1H-pyrazolo[3,4-d]pyrimidine as a white solid (1.2 g, 78%). ¹H NMR (400 MHz, CDCl₃) δ 9.06 (s, 1H), 8.22 (s, 1H), 6.11-6.03 (m, 1H), 4.19-4.10 (m, 1H), 3.89-3.79 (m, 1H), 2.66-2.53 (m, 1H), 2.22-2.12 (m, 1H), 2.01-1.93 (m, 1H), 1.85-1.73 (m, 2H), 1.72-1.67 (m, 1H).

Step B: (R)-3-Hydroxy-1-methyl-3-(3-(3-(1-(tetrahydro-2H-pyran-2-yl)-1H-pyrazolo[3,4-d]pyrimidin-6-yl)phenyl)isoxazol-5-yl)pyrrolidin-2-one. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 90 mg, 0.23 mmol), 6-chloro-1-(tetrahydro-2H-pyran-2-yl)-1H-pyrazolo[3,4-d]pyrimidine (61.5 mg, 0.26 mmol), K₃PO₄ (159 mg, 0.70 mmol), 1,4-dioxane (5.0 mL), and water (0.5 mL) was sparged with N₂ for 5 minutes, treated with Pd(dtbpf)Cl₂ (31 mg, 0.047 mmol), and then heated at 100° C. for 18 h under N₂ atmosphere. The reaction vessel was removed from the oil bath and allowed to gradually cool to rt. The mixture was then filtered through a pad of diatomaceous earth, and the pad washed with EtOAc (10 mL). The filtrate was concentrated to dryness in vacuo and the residue subjected to silica gel chromatography (0-100% EtOAc/petroleum ether) to give (R)-3-hydroxy-1-methyl-3-(3-(3-(1-(tetrahydro-2H-pyran-2-yl)-1H-pyrazolo[3,4-d]pyrimidin-6-yl)phenyl)isoxazol-5-yl)pyrrolidin-2-one as a yellow solid (70 mg, 65%). MS (ESI): Mass calcd. for C₂₄H₂₄N₆O₄ 460.18, m/z found 461.2 [M+H]⁺.

Step C: (R)-3-(3-(3-(1H-Pyrazolo[3,4-d]pyrimidin-6-yl)phenyl)isoxazol-5-yl)-3-hydroxy-1-methylpyrrolidin-2-one. A solution consisting of (R)-3-hydroxy-1-methyl-3-(3-(3-(1-(tetrahydro-2H-pyran-2-yl)-1H-pyrazolo[3,4-d]pyrimidin-6-yl)phenyl)isoxazol-5-yl)pyrrolidin-2-one (70 mg, 0.15 mmol), and MeOH (2.5 mL) was added HCl/1,4-dioxane (2.5 mL, 10 mmol, 4.0 M in 1,4-dioxane) in one portion. The resultant mixture was stirred for 2 h at room temperature and then concentrated to dryness in vacuo. The residue was subjected to HPLC (Boston Green ODS column, 5 μm, 150×30 mm, 25-55% (v/v) CH₃CN/H₂O with 0.225% FA) to afford, after lyophilization, (R)-3-(3-(3-(1H-pyrazolo[3,4-d]pyrimidin-6-yl)phenyl)isoxazol-5-yl)-3-hydroxy-1-methylpyrrolidin-2-one as a white solid (20.1 mg, 35%). MS (ESI): Mass calcd. for C₁₉H₁₆N₆O₃ 376.12, m/z found 377.1 [M+H]⁺. ¹H NMR (400 MHz, CDCl₃) δ 11.64 (br s, 1H), 9.27 (s, 1H), 8.79 (s, 1H), 8.54 (d, J=8.0 Hz, 1H), 8.20 (s, 1H), 7.93 (d, J=7.6 Hz, 1H), 7.59-7.50 (m, 1H), 6.88 (s, 1H), 4.84 (br s, 1H), 3.70-3.61 (m, 1H), 3.58-3.49 (m, 1H), 3.04 (s, 3H), 2.88-2.76 (m, 1H), 2.58-2.46 (m, 1H). ¹H NMR (400 MHz, DMSO-d₆) δ 14.16 (br s, 1H), 9.48 (s, 1H), 9.04-8.95 (m, 1H), 8.61 (d, J=8.0 Hz, 1H), 8.40 (s, 1H), 8.05 (d, J=7.6 Hz, 1H), 7.78-7.67 (m, 1H), 7.07 (s, 1H), 6.75 (s, 1H), 3.54-3.42 (m, 2H), 2.86 (s, 3H), 2.65-2.56 (m, 1H), 2.34-2.24 (m, 1H).

### Example 227: (R)-3-Hydroxy-1-methyl-3-(3-(3-(1-methyl-1H-pyrazolo[3,4-d]pyrimidin-6-yl)phenyl)isoxazol-5-yl)pyrrolidin-2-one

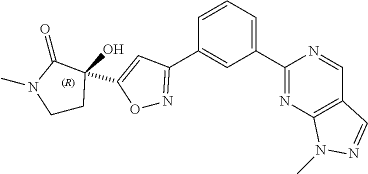

Step A: 6-Chloro-1-methyl-1H-pyrazolo[3,4-d]pyrimidine. A mixture consisting of 6-chloro-1H-pyrazolo[3,4-d]pyrimidine (500 mg, 3.2 mmol) and anhydrous DMF (8 mL) was subsequently evacuated and refilled with N₂ (×3), then treated with NaH (207 mg, 60 wt. %, 5.18 mmol) in five portions over 5 min at 0° C. The mixture was stirred for 30 min at 0° C., treated with Mel (0.25 mL in 2 mL of DMF) slowly over 2 min, then stirred for 1 h at 0° C. followed by stirring 1 h with gradual warming to room temperature. After this time, the reaction mixture was quenched with H₂O (20 mL) at 0° C. and then extracted with EtOAc (25 mL×3). The combined organic extracts were washed with water (20 mL×2) and brine (20 mL×2), dried over anhydrous Na₂SO₄, filtered, and concentrated to dryness in vacuo. The residue was subjected to silica gel chromatography (0-100% EtOAc/petroleum ether) to afford 6-chloro-1-methyl-1H-pyrazolo[3,4-d]pyrimidine as a pale yellow solid (450 mg, 83%). ¹H NMR (400 MHz, CDCl₃) δ 9.05 (s, 1H), 8.16 (s, 1H), 4.13 (s, 3H).

Step B: (R)-3-Hydroxy-1-methyl-3-(3-(3-(1-methyl-1H-pyrazolo[3,4-d]pyrimidin-6-yl)phenyl)isoxazol-5-yl)pyrrolidin-2-one. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 70 mg, 0.18 mmol), 6-chloro-1-methyl-1H-pyrazolo[3,4-d]pyrimidine (34 mg, 0.20 mmol), K₃PO₄ (124 mg, 0.55 mmol), 1,4-dioxane (5.0 mL), and water (0.5 mL) was subsequently evacuated and refilled with N₂ (×3), treated with Pd(dtbpf)Cl₂ (24 mg, 0.036 mmol), and heated at 100° C. for 18 h under a N₂ atmosphere. The reaction vessel was removed from the oil bath and allowed to gradually cool to rt. The mixture was then filtered through a pad of diatomaceous earth, and the pad washed with EtOAc (10 mL). The filtrate was concentrated in vacuo. The residue was subjected to HPLC (Boston Green ODS column, 5 μm, 150×30 mm; 30-60% (v/v) CH₃CN/H₂O with 0.225% FA) to afford, after lyophilization, (R)-3-hydroxy-1-methyl-3-(3-(3-(1-methyl-1H-pyrazolo[3,4-d]pyrimidin-6-yl)phenyl)isoxazol-5-yl)pyrrolidin-2-one (25.6 mg, 36%) as a white solid. MS (ESI): Mass calcd. for C₂₀H₁₈N₆O₃ 390.14, m/z found 391.1 [M+H]⁺. ¹H NMR (400 MHz, DMSO-d₆) δ 9.46 (s, 1H), 9.01 (s, 1H), 8.67 (d, J=8.0 Hz, 1H), 8.41 (s, 1H), 8.05 (d, J=8.0 Hz, 1H), 7.81-7.66 (m, 1H), 7.10 (s, 1H), 6.82 (s, 1H), 4.15 (s, 3H), 3.47-3.42 (m, 2H), 2.86 (s, 3H), 2.65-2.56 (m, 1H), 2.30 (s, 1H). ¹H NMR (400 MHz, CDCl₃) δ 9.21 (s, 1H), 8.92 (s, 1H), 8.62 (d, J=8.0 Hz, 1H), 8.12 (s, 1H), 7.92 (d, J=7.6 Hz, 1H), 7.61-7.51 (m, 1H), 6.86 (s, 1H), 4.30 (s, 1H), 4.19 (s, 3H), 3.72-3.62 (m, 1H), 3.55-3.44 (m, 1H), 3.01 (s, 3H), 2.85-2.76 (m, 1H), 2.55-2.43 (m, 1H).

### Example 228: (R)-3-(3-(3-(1H-Pyrazolo[4,3-b]pyridin-5-yl)phenyl)isoxazol-5-yl)-3-hydroxy-1-methylpyrrolidin-2-one

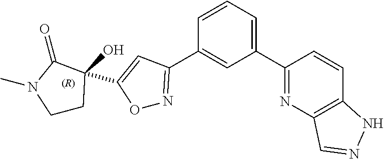

Step A: 5-Chloro-1-(tetrahydro-2H-pyran-2-yl)-1H-pyrazolo[4,3-b]pyridine. A mixture consisting of 5-chloro-1H-pyrazolo[4,3-b]pyridine (0.5 g, 3.3 mmol), pyridinium p-toluenesulfonate (136 mg, 0.33 mmol), 3,4-dihydro-2H-pyran (0.59 mL, 6.5 mmol), and CH₂Cl₂ (10.0 mL) was refluxed for 18 h under N₂ atmosphere. After this time, the reaction vessel was removed from the oil bath and allowed to gradually cool to rt. The mixture was then concentrated to dryness in vacuo and the residue subjected to silica gel chromatography (0-40% EtOAc/petroleum ether) to afford 5-chloro-1-(tetrahydro-2H-pyran-2-yl)-1H-pyrazolo[4,3-b]pyridine as a white solid (650 mg, 84%). MS (ESI): Mass calcd. for C₁₁H₁₂ClN₃O 237.06, m/z found 238.1 [M+H]⁺.

Step B: (R)-3-Hydroxy-1-methyl-3-(3-(3-(1-(tetrahydro-2H-pyran-2-yl)-1H-pyrazolo[4,3-b]pyridin-5-yl)phenyl)isoxazol-5-yl)pyrrolidin-2-one. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 190 mg, 0.026 mmol), 5-chloro-1-(tetrahydro-2H-pyran-2-yl)-1H-pyrazolo[4,3-b]pyridine (129 mg, 0.54 mmol), K₃PO₄ (336 mg, 1.48 mmol), 1,4-dioxane (5 mL), and water (0.5 mL) was subsequently evacuated and refilled with N₂ 3 times. Pd(dtbpf)Cl₂ (32 mg, 0.050 mmol) was added and then the reaction mixture was heated at 100° C. for 2 h under a N₂ atmosphere. After this time, the reaction vessel was removed from the oil bath and allowed to gradually cool to rt. The reaction mixture was filtered through a pad of diatomaceous earth, and the pad washed with CH₂Cl₂ (10 mL). The filtrate was concentrated to dryness in vacuo and the residue subjected to silica gel chromatography (0-100% EtOAc/petroleum ether) to afford (R)-3-hydroxy-1-methyl-3-(3-(3-(1-(tetrahydro-2H-pyran-2-yl)-1H-pyrazolo[4,3-b]pyridin-5-yl)phenyl)isoxazol-5-yl)pyrrolidin-2-one as a yellow solid (140 mg, 62%). MS (ESI): Mass calcd. for C₂₅H₂₅N₅O₄ 459.19, m/z found 460.2 [M+H]⁺.

Step C: (R)-3-(3-(3-(1H-Pyrazolo[4,3-b]pyridin-5-yl)phenyl)isoxazol-5-yl)-3-hydroxy-1-methylpyrrolidin-2-one. A solution consisting of (R)-3-hydroxy-1-methyl-3-(3-(3-(1-(tetrahydro-2H-pyran-2-yl)-1H-pyrazolo[4,3-b]pyridin-5-yl)phenyl)isoxazol-5-yl)pyrrolidin-2-one (140 mg, 0.31 mmol), and MeOH (5.0 mL) was added HCl/1,4-dioxane (5.0 mL, 4.0 M in 1,4-dioxane, 20 mmol) in one portion. The resultant mixture was stirred for 1 h at room temperature. After this time, the mixture was concentrated to dryness in vacuo and the residue subjected to HPLC (Boston Green ODS column, 5 m, 150×30 mm; 25-55% (v/v) CH₃CN/H₂O with 0.225% FA) to afford, after lyophilization, (R)-3-(3-(3-(1H-pyrazolo[4,3-b]pyridin-5-yl)phenyl)isoxazol-5-yl)-3-hydroxy-1-methylpyrrolidin-2-one as a white solid (49.4 mg, 43%). MS (ESI): Mass calcd. for C₂₀H₁₇N₅O₃ 375.13 m/z, found 376.2 [M+H]⁺. ¹H NMR (400 MHz, DMSO-d₆) δ 13.43 (br s, 1H), 8.62 (s, 1H), 8.41 (s, 1H), 8.28 (d, J=8.0 Hz, 1H), 8.19-8.08 (m, 2H), 7.95 (d, J=8.0 Hz, 1H), 7.70-7.62 (m, 1H), 7.16 (s, 1H), 6.77 (s, 1H), 3.54-3.47 (m, 2H), 2.86 (s, 3H), 2.64-2.56 (m, 1H), 2.35-2.23 (m, 1H). ¹H NMR (400 MHz, CD₃CN) δ 11.44 (br s, 1H), 8.60-8.51 (m, 1H), 8.29-8.23 (m, 1H), 8.23-8.16 (m, 1H), 8.08-8.03 (m, 1H), 7.98-7.93 (m, 1H), 7.92-7.86 (m, 1H), 7.66-7.58 (m, 1H), 6.90 (s, 1H), 4.65 (br s, 1H), 3.54-3.44 (m, 2H), 2.89 (s, 3H), 2.73-2.63 (m, 1H), 2.41-2.30 (m, 1H).

### Example 229: (R)-3-Hydroxy-1-methyl-3-(3-(3-(1-methyl-1H-pyrazolo[4,3-b]pyridin-5-yl)phenyl)isoxazol-5-yl)pyrrolidin-2-one

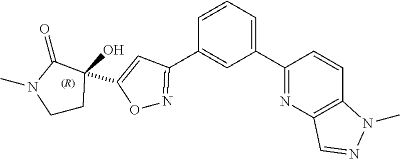

Step A: 5-Chloro-1-methyl-1H-pyrazolo[4,3-b]pyridine. A mixture consisting of 5-chloro-1H-pyrazolo[4,3-b]pyridine (1.0 g, 6.5 mmol) and anhydrous THF (22 mL) was subsequently evacuated and refilled with N₂ for 3 times, treated with NaH (418 mg, 10.4 mmol, 60 wt. %) portion wise at 0° C., and then stirred for 30 min at 0° C. The mixture was further treated with Mel (0.5 mL, 8 mmol, in 3 mL of THF) and stirred for 1 h at 0° C., and then stirred for 1 h with gradual warming to room temperature. After this time, the reaction was quenched with H₂O (20 mL) at 0° C. and extracted with EtOAc (25 mL×3). The combined organic extracts were washed with water (20 mL×2) and brine (20 mL×2), dried over anhydrous Na₂SO₄, filtered, and concentrated to dryness in vacuo. The residue was subjected to silica gel chromatography (0-30% EtOAc/petroleum ether) to afford 5-chloro-1-methyl-1H-pyrazolo[4,3-b]pyridine as a pale yellow solid (400 mg, 37%). ¹H NMR (400 MHz, CDCl₃) δ 8.13 (s, 1H), 7.73 (d, J=8.8 Hz, 1H), 7.30 (d, J=8.8 Hz, 1H), 4.11 (s, 3H).

Step B: (R)-3-Hydroxy-1-methyl-3-(3-(3-(1-methyl-1H-pyrazolo[4,3-b]pyridin-5-yl)phenyl)isoxazol-5-yl)pyrrolidin-2-one. A mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 180 mg, 0.47 mmol), 5-chloro-1-methyl-1H-pyrazolo[4,3-b]pyridine (86.4 mg, 0.52 mmol), K₃PO₄ (319 mg, 1.41 mmol), 1,4-dioxane (5.0 mL), and water (0.5 mL) was subsequently evacuated and refilled with N₂ for 3 times, treated with Pd(dtbpf)Cl₂ (31 mg, 0.047 mmol), and heated at 100° C. for 18 h under a N₂ atmosphere. After this time, the reaction vessel was removed from the oil bath and allowed to gradually cool to rt. The mixture was filtered through a pad of diatomaceous earth and the pad washed with CH₂Cl₂ (10 mL). The filtrate was concentrated in vacuo and the residue subjected to HPLC (Boston Green ODS column, 5 μm, 150×30 mm, 28-58% (v/v) CH₃CN/H₂O with 0.225% FA) to afford, after lyophilization, the title compound (57.3 mg, 31%) as a white solid. MS (ESI): Mass calcd. for C₂₁H₁₉N₅O₃ 389.14, m/z found 390.3 [M+H]⁺. ¹H NMR (400 MHz, CDCl₃) δ 8.41 (s, 1H), 8.28 (s, 1H), 8.13 (d, J=8.0 Hz, 1H), 7.87-7.78 (m, 3H), 7.60-7.53 (m, 1H), 6.81 (s, 1H), 4.13 (s, 3H), 4.04 (s, 1H), 3.71-3.61 (m, 1H), 3.53-3.45 (m, 1H), 3.01 (s, 3H), 2.85-2.75 (m, 1H), 2.54-2.43 (m, 1H). ¹H NMR (400 MHz, DMSO-d₆) δ 8.62 (s, 1H), 8.37 (s, 1H), 8.29 (d, J=8.4 Hz, 2H), 8.18 (d, J=9.2 Hz, 1H), 7.95 (d, J=8.0 Hz, 1H), 7.71-7.61 (m, 1H), 7.16 (s, 1H), 6.76 (s, 1H), 4.13 (s, 3H), 3.56-3.45 (m, 2H), 2.86 (s, 3H), 2.64-2.55 (m, 1H), 2.35-2.23 (m, 1H).

### Example 230: (R)-3-Hydroxy-3-(3-(3-(4-methoxy-1,3-dimethyl-1H-pyrazolo[3,4-b]pyridin-6-yl)phenyl)isoxazol-5-yl)-1-methylpyrrolidin-2-one

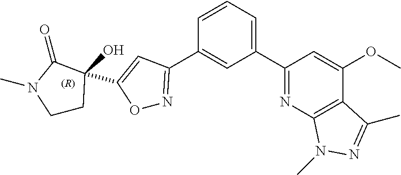

Step A: 1,3-Dimethyl-1H-pyrazolo[3,4-b]pyridine-4,6(5H,7H)-dione. A mixture consisting of diethyl malonate (4.00 mL, 26.3 mmol) and 1,3-dimethyl-1H-pyrazol-5-amine (2.00 g, 18.0 mmol) was heated at 180° C. for 5 h. After this time, the reaction vessel was removed from the oil bath and allowed to gradually cool to rt. The mixture was poured into water (20 mL) and filtered. The filter cake was washed with water (10 mL), and concentrated in vacuo to afford 1,3-dimethyl-1H-pyrazolo[3,4-b]pyridine-4,6(5H,7H)-dione as a yellow solid (4.96 g). ¹H NMR (400 MHz, DMSO-d₆) δ 11.50-10.99 (m, 2H), 5.35 (s, 1H), 3.67 (s, 3H), 2.29 (s, 3H).

Step B: 4,6-Dichloro-1,3-dimethyl-1H-pyrazolo[3,4-b]pyridine. 1,3-Dimethyl-1H-pyrazolo[3,4-b]pyridine-4,6(5H,7H)-dione (2.48 g, 13.8 mmol) was added into phenylphosphonic dichloride (15.0 mL, 106 mmol, 1.38 g/mL) and the resultant mixture was heated at 150° C. for 3 h. After this time, the reaction vessel was removed from the oil bath and allowed to gradually cool to rt. The mixture was poured into ice water, brought to pH 8-9 with NH₄OH (25 wt. % in H₂O), and extracted with EtOAc (60 mL×3). The combined organic extracts were washed with brine (80 mL), dried over anhydrous Na₂SO₄, filtered, and concentrated to dryness in vacuo. The residue was subjected to silica gel chromatography (0-50% EtOAc/petroleum ether) to afford 4,6-dichloro-1,3-dimethyl-1H-pyrazolo[3,4-b]pyridine as a white solid (2.0 g, 65%). MS (ESI): Mass calcd. for C₈H₇Cl₂N₃ 215.00 m/z, found 215.9 [M+H]⁺.

Step C: 6-Chloro-4-methoxy-1,3-dimethyl-1H-pyrazolo[3,4-b]pyridine. Sodium methoxide (800 mg, 14.8 mmol) was added into a solution consisting of 4,6-dichloro-1,3-dimethyl-1H-pyrazolo[3,4-b]pyridine (1.00 g, 4.63 mmol) in CH₃OH (15 mL). The resultant mixture was stirred for 16 h at room temperature. After this time, the mixture was concentrated to dryness in vacuo. The residue was subjected to silica gel chromatography (0-50% EtOAc/petroleum ether) to afford 6-chloro-4-methoxy-1,3-dimethyl-1H-pyrazolo[3,4-b]pyridine as a white solid (0.8 g, 82%). ¹H NMR (400 MHz, DMSO-d₆) δ 6.75 (s, 1H), 6.78-6.72 (m, 1H), 4.01 (s, 3H), 3.86 (s, 3H), 2.47 (s, 3H).

Step D: (R)-3-Hydroxy-3-(3-(3-(4-methoxy-1,3-dimethyl-1H-pyrazolo[3,4-b]pyridin-6-yl)phenyl)isoxazol-5-yl)-1-methylpyrrolidin-2-one. 6-Chloro-4-methoxy-1,3-dimethyl-1H-pyrazolo[3,4-b]pyridine (90 mg, 0.43 mmol) was added into a mixture consisting of (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 135 mg, 0.351 mmol) and K₃PO₄ (225 mg, 1.06 mmol) in 1,4-dioxane (8 mL) and H₂O (2 mL). The resultant mixture was subsequently evacuated and refilled with N₂ 3 times, treated with Pd(dtbpf)Cl₂ (45 mg, 0.069 mmol), and heated at 100° C. for 16 h. After this time, the reaction vessel was removed from the oil bath and allowed to gradually cool to rt. The mixture was diluted with H₂O (15 mL) and extracted with ethyl acetate (20 mL×3). The combined organic extracts were washed with brine, dried with anhydrous Na₂SO₄, filtered, and concentrated to dryness in vacuo. The residue was subjected to HPLC (Boston Prime C18 column, 5 μm, 150 mm×30 mm; 35-65% (v/v) CH₃CN/H₂O with 0.05% NH₃H₂O+10 mM NH₄HCO₃) to afford, after lyophilization, (R)-3-hydroxy-3-(3-(3-(4-methoxy-1,3-dimethyl-1H-pyrazolo[3,4-b]pyridin-6-yl)phenyl)isoxazol-5-yl)-1-methylpyrrolidin-2-one as a white solid (60.3 mg, 39%). MS (ESI): Mass calcd. for C₂₃H₂₃N₅O₄ 433.17 m/z found 434.1 [M+H]⁺. ¹H NMR (400 MHz, DMSO-d₆) δ 8.65-8.59 (m, 1H), 8.39-8.32 (m, 1H), 8.01-7.95 (m, 1H), 7.73-7.63 (m, 1H), 7.29 (s, 1H), 7.16 (s, 1H), 6.76 (s, 1H), 4.14 (s, 3H), 4.00 (s, 3H), 3.54-3.40 (m, 2H), 2.86 (s, 3H), 2.63-2.57 (m, 1H), 2.55 (s, 3H), 2.32-2.25 (m, 1H).

### Example 231: (R)-2-(3-(5-(3-Hydroxy-1-methyl-2-oxopyrrolidin-3-yl)isoxazol-3-yl)phenyl)-5-(pyrrolidin-1-ylmethyl)thiazole-4-carboxamide

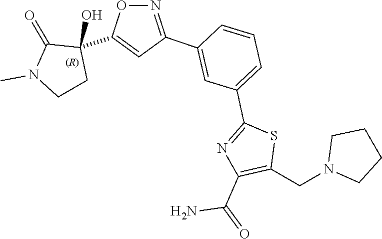

Step A: (R)-Methyl 5-formyl-2-(3-(5-(3-hydroxy-1-methyl-2-oxopyrrolidin-3-yl)isoxazol-3-yl)phenyl)thiazole-4-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 250 mg, 0.651 mmol), methyl 2-bromo-5-formylthiazole-4-carboxylate (325 mg, 1.30 mmol, Example 222 Step B), Pd(dtbpf)Cl₂ (85 mg, 0.13 mmol), K₃PO₄ (552 mg, 2.60 mmol), H₂O (0.5 mL), and 1,4-dioxane (5 mL) were added to a 40 mL vial. The vial was subsequently evacuated and refilled with N₂ 3 times. The vial was capped and heated at 80° C. for 2 h. The reaction vessel was removed from the heating device and allowed to gradually cool to rt. The mixture was filtered through a pad of diatomaceous earth. The filtrate was concentrated to dryness in vacuo to give a brown gum. The gum was subjected to silica gel chromatography (0-5% MeOH/CH₂Cl₂) to give (R)-methyl 5-formyl-2-(3-(5-(3-hydroxy-1-methyl-2-oxopyrrolidin-3-yl)isoxazol-3-yl)phenyl)thiazole-4-carboxylate as a light brown solid (200 mg, 36%). MS (ESI): Mass calcd. for C₂₀H₁₇N₃O₆S 427.08 m/z, found 428.1 [M+H]⁺.

Step B: (R)-Methyl 2-(3-(5-(3-hydroxy-1-methyl-2-oxopyrrolidin-3-yl)isoxazol-3-yl)phenyl)-5-(pyrrolidin-1-ylmethyl)thiazole-4-carboxylate. (R)-Methyl 5-formyl-2-(3-(5-(3-hydroxy-1-methyl-2-oxopyrrolidin-3-yl)isoxazol-3-yl)phenyl)thiazole-4-carboxylate (150 mg, 0.351 mmol), pyrrolidine (89 μL, 0.70 mmol), titanium(IV) isopropoxide (110 mg, 0.386 mmol), NaBH₃CN (55 mg, 0.88 mmol), and MeOH (8 mL) were added to a 40 mL vial, and the resulting mixture was stirred for 18 h at 60° C. The reaction vessel was removed from the heating device and allowed to gradually cool to rt. The mixture was filtered through a pad of diatomaceous earth, and the filtrate concentrated to dryness in vacuo to give a brown gum. The gum was subjected to silica gel chromatography (0-5% MeOH/CH₂Cl₂) to give (R)-methyl 2-(3-(5-(3-hydroxy-1-methyl-2-oxopyrrolidin-3-yl)isoxazol-3-yl)phenyl)-5-(pyrrolidin-1-ylmethyl)thiazole-4-carboxylate as a light brown solid (120 mg, 71%). MS (ESI): Mass calcd. for C₂₄H₂₆N₄O₅S 482.16 m/z, found 483.2 [M+H]⁺.

Step C: (R)-2-(3-(5-(3-Hydroxy-1-methyl-2-oxopyrrolidin-3-yl)isoxazol-3-yl)phenyl)-5-(pyrrolidin-1-ylmethyl)thiazole-4-carboxamide. (R)-Methyl 2-(3-(5-(3-hydroxy-1-methyl-2-oxopyrrolidin-3-yl)isoxazol-3-yl)phenyl)-5-(pyrrolidin-1-ylmethyl)thiazole-4-carboxylate (100 mg, 0.207 mmol), NH₃ (8 mL, 56 mmol, 7 M in MeOH), and MeOH (2.5 mL) were added to a 40 mL vial, and the resulting mixture stirred for 18 h at 75° C. The reaction vessel was removed from the heating device and allowed to gradually cool to rt. The mixture was concentrated in vacuo to give a brown gum, which was subjected to HPLC (Boston Green ODS column, 5 μm, 150×30 mm; 9-39% CH₃CN/H₂O with 0.225% FA), and then subjected to SFC (DAICEL CHIRALCEL® AS column, 10 μm, 250×30 mm; 65% EtOH (containing 0.1% NH₃H₂O)/supercritical CO₂) to give, after lyophilization, (R)-2-(3-(5-(3-hydroxy-1-methyl-2-oxopyrrolidin-3-yl)isoxazol-3-yl)phenyl)-5-(pyrrolidin-1-ylmethyl)thiazole-4-carboxamide as a white solid (43.6 mg, 66%). MS (ESI): Mass calcd. for C₂₃H₂₅N₅O₄S 467.16 m/z, found 468.2 [M+H]⁺. ¹H NMR (400 MHz, DMSO-d₆) δ 8.48 (s, 1H), 8.13 (d, J=7.2 Hz, 1H), 8.01 (d, J=7.6 Hz, 2H), 7.70-7.58 (m, 2H), 7.19 (s, 1H), 6.74 (s, 1H), 4.29 (br s, 2H), 3.54-3.44 (m, 2H), 2.85 (s, 3H), 2.75-2.61 (m, 4H), 2.59-2.54 (m, 1H), 2.35-2.24 (m, 1H), 1.81-1.71 (m, 4H). ¹H NMR (400 MHz, CD₃OD) δ 8.52-8.47 (m, 1H), 8.09 (d, J=8.0 Hz, 1H), 7.97 (d, J=8.0 Hz, 1H), 7.67-7.57 (m, 1H), 7.02 (s, 1H), 4.44 (s, 2H), 3.63-3.54 (m, 2H), 2.97 (s, 3H), 2.87-2.80 (m, 4H), 2.79-2.70 (m, 1H), 2.45-2.35 (m, 1H), 1.93-1.83 (m, 4H).

### Example 232: (R)-3-(3-(5-(3-Hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxamide

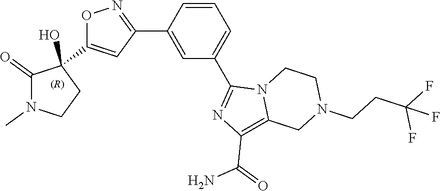

Step A: Ethyl 3-bromo-7-(3,3,3-trifluoropropyl)-5,6,7,8-tetrahydroimidazo[1,5-a]pyrazine-1-carboxylate. Ethyl 3-bromo-5,6,7,8-tetrahydroimidazo[1,5-a]pyrazine-1-carboxylate (290 mg, 0.934 mmol, Intermediate 64), 1,1,1-trifluoro-3-iodopropane (547 μL, 4.67 mmol), and DMF (3 mL) were added to a 20 mL microwave tube, and charged with DIEA (813 μL, 4.67 mmol). The resulting mixture was stirred for 2 h at 120° C. via microwave irradiation. The vessel was removed from the heating device and allowed to gradually cool to rt. The mixture was diluted with water (10 mL) and extracted with EtOAc (10 mL×2). The combined organic extracts were dried over anhydrous Na₂SO₄, filtered, and concentrated to dryness in vacuo to give a yellow oil. The oil was then subjected to silica gel chromatography (0-50% EtOAc/petroleum ether) to give ethyl 3-bromo-7-(3,3,3-trifluoropropyl)-5,6,7,8-tetrahydroimidazo[1,5-a]pyrazine-1-carboxylate as a yellow oil (300 mg, 86%). LCMS (ESI): Mass calcd. for Cl₂H₁₅BrF₃N₃O₂ 369.0 m/z, found 369.9 [M+H]⁺.

Step B: (R)-Ethyl 3-(3-(5-(3-hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylate. Ethyl 3-bromo-7-(3,3,3-trifluoropropyl)-5,6,7,8-tetrahydroimidazo[1,5-a]pyrazine-1-carboxylate (200 mg, 0.540 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 200 mg, 0.521 mmol), K₃PO₄ (344 mg, 1.62 mmol) and 1,4-dioxane/H₂O=4:1 (2 mL) were added to an oven-dried and N₂-purged 8 mL microwave tube. The mixture was sparged with N₂ for 5 min, treated with Pd(dtbpf)Cl₂ (35 mg, 0.054 mmol), and heated to 80° C. for 1 hour via microwave irradiation. After this time, the reaction vessel was removed from the heating device and allowed to gradually cool to rt. The reaction mixture was then diluted with H₂O (5 mL) and extracted with EtOAc (5 mL×3). The combined organic extracts were washed with brine (5 mL), dried over anhydrous Na₂SO₄, filtered, and concentrated to dryness in vacuo to give a brown oil.

The oil was then subjected to silica gel chromatography (0-100% EtOAc/petroleum ether) to give (R)-ethyl 3-(3-(5-(3-hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylate as an orange solid (170 mg, 57%). LCMS (ESI): Mass calcd. for C₂₆H₂₈F₃N₅O₅ 547.20 m/z, found 548.3 [M+H]⁺.

Step C: (R)-3-(3-(5-(3-Hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylic acid. (R)-ethyl 3-(3-(5-(3-hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylate (130 mg, 0.237 mmol), LiOH·H₂O (60 mg, 1.4 mmol), H₂O (1 mL) and anhydrous THF (2 mL) were added to an 8 mL vial. The resulting mixture was stirred for 16 h at 40° C. After this time the reaction mixture was acidified with 1.0 M aqueous HCl to pH=2 and extracted with DCM (20 mL×2). The combined organic extracts were washed with brine, dried over anhydrous Na₂SO₄, filtered, and concentrated to dryness in vacuo to give a yellow solid. The solid was then subjected to silica gel chromatography (0-100% EtOAc/petroleum ether followed by 0-10% MeOH/EtOAc) to give (R)-3-(3-(5-(3-hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylic acid as a yellow oil (70 mg, 50%). LCMS (ESI): Mass calcd. for C₂₄H₂₄F₃N₅O₅ 519.17 m/z, found 520.2 [M+1]⁺.

Step D: (R)-3-(3-(5-(3-Hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxamide. (R)-3-(3-(5-(3-Hydroxy-1-methyl-2-oxopyrrolidin-3-yl)isoxazol-3-yl)phenyl)-7-(3,3,3-trifluoropropyl)-5,6,7,8-tetrahydroimidazo[1,5-a]pyrazine-1-carboxylic acid (70 mg, 0.14 mmol) and DMF (1 mL) were added to an 8 mL vial, and charged with TEA (56 μL, 0.40 mmol), HATU (61 mg, 0.16 mmol) and NH₄Cl (72 mg, 1.3 mmol). The mixture was stirred at room temperature for 16 h. After this time, the mixture was poured into water (5 mL) and extracted with EtOAc (5 mL×3). The combined organic extracts were washed with brine (5 mL), dried over anhydrous Na₂SO₄, filtered, and concentrated to dryness under reduced pressure to afford a brown oil. The oil was then subjected to HPLC (Welch Xtimate C18 column, 5 μm, 150×30 mm; 25-55% (v/v) MeCN/water (0.05% NH₃H₂O+10 mM NH₄HCO₃)) to give a colorless solid, which was further purified by SFC (DAICEL CHIRALCEL® OD-H column, 5 μm, 250×30 mm; 40% (v/v) EtOH (0.1% NH₃H₂O)/CO₂)) to give the title compound as a colorless solid (15.1 mg, 50%). LCMS (ESI): Mass calcd. for C₂₄H₂₅F₃N₆O₄ 518.19 m/z, found 519.2 [M+1]⁺. ¹H NMR (400 MHz, DMSO-d₆) δ 8.22 (s, 1H), 7.96 (d, J=7.5 Hz, 1H), 7.88 (d, J=7.5 Hz, 1H), 7.68-7.59 (m, 1H), 7.33 (br s, 1H), 7.17-7.04 (m, 2H), 6.72 (s, 1H), 4.26-4.16 (m, 2H), 4.00 (s, 2H), 3.47-3.40 (m, 2H), 2.90-2.85 (m, 2H), 2.85-2.79 (m, 5H), 2.62-2.54 (m, 3H), 2.31-2.22 (m, 1H). ¹⁹F NMR (376 MHz, DMSO-d₆) δ −63.42 (s, 3F).

### Example 233: (R)-3-(3-(5-(3-Hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxamide

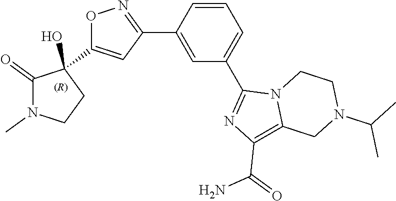

Step A: Ethyl 3-bromo-7-isopropyl-5,6,7,8-tetrahydroimidazo[1,5-a]pyrazine-1-carboxylate. Ethyl 3-bromo-5,6,7,8-tetrahydroimidazo[1,5-a]pyrazine-1-carboxylate (220 mg, 0.80 mmol, Intermediate 64), and MeOH (6 mL) were added to a 40 mL flask, and then propan-2-one (0.9 mL, 12.3 mmol) and AcOH (138 μL, 2.41 mmol) were added. The mixture was stirred at room temperature for 0.5 h, treated with NaBH₃CN (150 mg, 2.39 mmol), and heated at 45° C. for 3 h. After this time, the mixture was concentrated to dryness in vacuo to give a black solid. The black solid was subjected to silica gel chromatography (0-100% EtOAc/petroleum ether) to give ethyl 3-bromo-7-isopropyl-5,6,7,8-tetrahydroimidazo[1,5-a]pyrazine-1-carboxylate as a yellow solid (100 mg, 35%). LCMS (ESI): Mass calcd. for C₁₂H₁₈BrN₃O₂ 315.06 m/z, found 316.0 [M+1]⁺.

Step B: (R)-Ethyl 3-(3-(5-(3-hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 120 mg, 0.312 mmol), 1,4-dioxane (1.6 mL), and H₂O (0.4 mL) were added to a nitrogen-purged 8 mL vial, and then ethyl 3-bromo-7-isopropyl-5,6,7,8-tetrahydroimidazo[1,5-a]pyrazine-1-carboxylate (90 mg, 0.29 mmol), Pd(dtbpf)Cl₂ (28 mg, 0.043 mmol), and K₃PO₄ (186 mg, 0.876 mmol) were added. The resulting mixture was heated at 100° C. for 6 h. The reaction vessel was removed from the heating device and allowed to gradually cool to RT. The reaction mixture was diluted with H₂O (20 mL) and extracted with ethyl acetate (10 mL×3). The combined organic extracts were dried with anhydrous Na₂SO₄, filtered, and concentrated to dryness in vacuo to give a nearly black viscous oil. The oil was then subjected to silica gel chromatography (0-100% EtOAc/petroleum ether) to give (R)-ethyl 3-(3-(5-(3-hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylate as a red solid (130 mg, 85%). LCMS (ESI): Mass calcd. for C₂₆H₃₁N₅O₅ 493.23 m/z, found 494.3 [M+1]⁺.

Step C: (R)-3-(3-(5-(3-Hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylic acid. (R)-Ethyl 3-(3-(5-(3-hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylate (110 mg, 0.223 mmol), THF (4 mL), EtOH (0.4 mL), and H₂O (1 mL) were added to a nitrogen-purged 50 mL round-bottomed flask, and then LiOH·H₂O (100 mg, 2.38 mmol) was added. The mixture was stirred at rt for 16 h. The reaction mixture was acidified to pH=2 with 1.0 M aqueous HCl and extracted with DCM (20 mL×2). The combined organic extracts were washed with brine, dried over Na₂SO₄, filtered, and concentrated to dryness in vacuo to give (R)-3-(3-(5-(3-hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylic acid (90 mg, 85%) as a pale-yellow solid. The product was used to next step directly without further purification. LCMS (ESI): Mass calcd. for C₂₄H₂₇N₅O₅ 465.20 m/z, found 466.1 [M+1]⁺.

Step D: (R)-3-(3-(5-(3-Hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxamide. (R)-3-(3-(5-(3-Hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxylic acid (80 mg, 0.17 mmol) and DMF (2 mL) were added to a nitrogen-purged 8 mL vial, and then TEA (90 μL, 0.65 mmol), HATU (95 mg, 0.25 mmol), and NH₄Cl (100 mg, 1.87 mmol) were added. The resulting mixture was stirred at room temperature for 6 h. After this time, the reaction mixture was filtered to give a yellow solution. The solution was then subjected to HPLC ((Boston Prime C18 column, 5 μm, 150×30 mm; 20-50% ACN/water (0.05% NH₃H₂O+10 mM NH₄HCO₃)) to yield (R)-3-(3-(5-(3-hydroxy-1-methyl-2-oxopyrrolidin-3-yl)isoxazol-3-yl)phenyl)-7-isopropyl-5,6,7,8-tetrahydroimidazo[1,5-a]pyrazine-1-carboxamide as a white solid (10.8 mg, 13%). LCMS (ESI): Mass calcd. for C₂₄H₂₈N₆O₄ 464.22 m/z found 465.3 [M+1]⁺. ¹H NMR (400 MHz, DMSO-d₆) δ 8.31-8.24 (m, 1H), 8.05-7.99 (m, 1H), 7.96-7.90 (m, 1H), 7.73-7.66 (m, 1H), 7.37 (br s, 1H), 7.18 (s, 1H), 7.13 (br s, 1H), 6.79 (s, 1H), 4.32-4.19 (m, 2H), 4.05 (s, 2H), 3.57-3.51 (m, 2H), 3.06-2.95 (m, 1H), 2.93-2.85 (m, 5H), 2.66-2.62 (m, 1H), 2.38-2.27 (m, 1H), 1.14 (d, J=6.4 Hz, 6H).

### Example 234: (R)-7-Benzyl-3-(3-(5-(3-hydroxy-1-methyl-2-oxopyrrolidin-3-yl)isoxazol-3-yl)phenyl)-5,6,7,8-tetrahydroimidazo[1,5-a]pyrazine-1-carboxamide

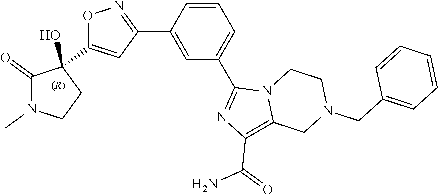

Step A: Ethyl 7-benzyl-3-bromo-5,6,7,8-tetrahydroimidazo[1,5-a]pyrazine-1-carboxylate. Ethyl 3-bromo-5,6,7,8-tetrahydroimidazo[1,5-a]pyrazine-1-carboxylate (480 mg, 1.75 mmol, Intermediate 64), (bromomethyl)benzene (0.43 mL, 3.6 mmol), TEA (0.19 mL, 1.4 mmol), and THF (24 mL) were added to a 100 mL round-bottomed flask, and the resulting mixture stirred for 1 h at room temperature. The reaction mixture was then subjected to silica gel chromatography (0-50% EtOAc/petroleum ether) to give ethyl 7-benzyl-3-bromo-5,6,7,8-tetrahydroimidazo[1,5-a]pyrazine-1-carboxylate as a colorless oil (210 mg, 30%). LCMS (ESI): Mass calcd. for C₁₆H₁₈BrN₃O₂ 363.06 m/z, found 363.8 [M+1]⁺.

Step B: (R)-Ethyl 7-benzyl-3-(3-(5-(3-hydroxy-1-methyl-2-oxopyrrolidin-3-yl)isoxazol-3-yl)phenyl)-5,6,7,8-tetrahydroimidazo[1,5-a]pyrazine-1-carboxylate. Ethyl 7-benzyl-3-bromo-5,6,7,8-tetrahydroimidazo[1,5-a]pyrazine-1-carboxylate (190 mg, 0.522 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 399 mg, 1.04 mmol), K₃PO₄ (333 mg, 1.57 mmol), and 1,4-dioxane/H₂O (v/v=4:1, 10 mL) were added to a 20 mL microwave tube, and charged with Pd(dtbpf)Cl₂ (38 mg, 0.058 mmol). The resulting mixture was sparged with N₂ for 5 min and stirred for 1 h at 90° C. via microwave irradiation. The reaction vessel was removed from the heating device and allowed to gradually cool to rt. The reaction mixture was diluted with water (15 mL), extracted with ethyl acetate (20 mL×3), and the combined extracts were treated with brine (20 mL), dried over anhydrous Na₂SO₄, filtered, and concentrated to dryness in vacuo to give a white solid. The white solid was then subjected to silica gel chromatography (0-100% EtOAc/petroleum ether) to give (R)-ethyl 7-benzyl-3-(3-(5-(3-hydroxy-1-methyl-2-oxopyrrolidin-3-yl)isoxazol-3-yl)phenyl)-5,6,7,8-tetrahydroimidazo[1,5-a]pyrazine-1-carboxylate as a colorless oil (300 mg, 83%). LCMS (ESI): Mass calcd. for C₃₀H₃₁N₅O₅ 541.23 m/z, found 542.1 [M+1]⁺.

Step C: (R)-7-Benzyl-3-(3-(5-(3-hydroxy-1-methyl-2-oxopyrrolidin-3-yl)isoxazol-3-yl)phenyl)-5,6,7,8-tetrahydroimidazo[1,5-a]pyrazine-1-carboxamide. (R)-Ethyl 7-benzyl-3-(3-(5-(3-hydroxy-1-methyl-2-oxopyrrolidin-3-yl)isoxazol-3-yl)phenyl)-5,6,7,8-tetrahydroimidazo[1,5-a]pyrazine-1-carboxylate (260 mg, 0.480 mmol), and NH₃ (20 mL, 7 M in MeOH) were added to a 50 mL round-bottomed flask, and the resulting mixture stirred for 48 h at 70° C. After this time, the mixture was cooled to r.t. and concentrated to dryness in vacuo to give a colorless oil. The colorless oil was subjected to HPLC (Welch Xtimate C18 column, 5 μm, 150×30 mm; 25-55% ACN/water (0.05% NH₃H₂O+10 mM NH₄HCO₃)) to give a colorless solid. The colorless solid was then subjected to SFC (DAICEL CHIRALPAK® AD column, 10 μm, 250×30 mm; 45% IPA (containing 0.1% of 25% aq. NH₃)/CO₂) to give the title compound as a colorless solid (20.4 mg, 41%). LCMS (ESI): Mass calcd. for C₂₈H₂₈N₆O₄ 512.22 m/z, found 513.3 [M+H]⁺. ¹H NMR (400 MHz, DMSO-d₆) δ 8.22 (s, 1H), 7.98-7.94 (m, 1H), 7.91-7.86 (m, 1H), 7.67-7.60 (m, 1H), 7.43-7.35 (m, 4H), 7.34-7.28 (m, 2H), 7.13 (s, 1H), 7.06 (br s, 1H), 6.73 (s, 1H), 4.29-4.21 (m, 2H), 3.96-3.91 (m, 2H), 3.79-3.75 (m, 2H), 3.53-3.41 (m, 2H), 2.89-2.76 (m, 5H), 2.61-2.54 (m, 1H), 2.33-2.23 (m, 1H).

### Example 235: (R)-5-((5-Cyano-2-methylpyridin-3-yl)amino)-2-(3-(5-(3-hydroxy-1-methyl-2-oxopyrrolidin-3-yl)isoxazol-3-yl)phenyl)thiazole-4-carboxamide

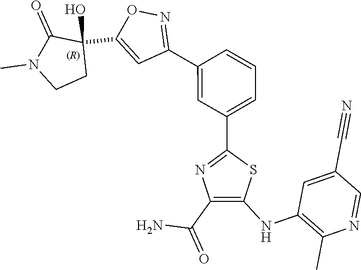

Step A: Ethyl 5-((5-cyano-2-methylpyridin-3-yl)amino)thiazole-4-carboxylate. 5-Chloro-6-methylnicotinonitrile (130 mg, 0.852 mmol), ethyl 5-aminothiazole-4-carboxylate (220 mg, 1.28 mmol), K₂CO₃ (353 mg, 2.56 mmol) and t-BuOH (3 mL) were added to a 5 mL microwave tube. The resultant mixture was sparged with N₂ for 5 min and then treated with BrettPhos-Pd-G3 (77 mg, 0.085 mmol) and BrettPhos (46 mg, 0.085 mmol). The resulting mixture was sparged with N₂ for another 5 min and then heated at 110° C. via microwave for 1 h. After this time, the reaction vessel was removed from the heating device and allowed to gradually cool to room temperature. The reaction mixture was concentrated to dryness in vacuo to give a brown solid, which was then subjected to silica gel chromatography (0-50% EtOAc/petroleum ether) to give ethyl 5-((5-cyano-2-methylpyridin-3-yl)amino)thiazole-4-carboxylate as a yellow solid (246 mg, 92%). LCMS (ESI): Mass calcd. for C₁₃H₁₂N₄O₂S 288.07 m/z, found 289.1 [M+H]⁺.

Step B: Ethyl 2-bromo-5-((5-cyano-2-methylpyridin-3-yl)amino)thiazole-4-carboxylate. Ethyl 5-((5-cyano-2-methylpyridin-3-yl)amino)thiazole-4-carboxylate (220 mg, 0.763 mmol), and MeCN (5 mL) were added to a 40 mL flask, which was charged with NBS (272 mg, 1.53 mmol). The mixture was stirred for 16 h at room temperature. The mixture was concentrated to dryness in vacuo to give a yellow solid, which was then subjected to silica gel chromatography (0-30% EtOAc/petroleum ether) to give ethyl 2-bromo-5-((5-cyano-2-methylpyridin-3-yl)amino)thiazole-4-carboxylate as a yellow solid (177 mg, 58%). LCMS (ESI): Mass calcd. for C₁₃H₁₁BrN₄O₂S 365.98 m/z, found 367.0 [M+1]⁺. ¹H NMR (400 MHz, DMSO-d₆) δ 9.49 (s, 1H), 8.74-8.67 (m, 1H), 8.25-8.20 (m, 1H), 4.29 (q, J=7.0 Hz, 2H), 2.57 (s, 3H), 1.27 (t, J=7.1 Hz, 3H).

Step C: (R)-Ethyl 5-((5-cyano-2-methylpyridin-3-yl)amino)-2-(3-(5-(3-hydroxy-1-methyl-2-oxopyrrolidin-3-yl)isoxazol-3-yl)phenyl)thiazole-4-carboxylate. Ethyl 2-bromo-5-((5-cyano-2-methylpyridin-3-yl)amino)thiazole-4-carboxylate (100 mg, 0.272 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 126 mg, 0.327 mmol), K₃PO₄ (173 mg, 0.817 mmol) and 1,4-dioxane/H₂O (4:1, 5 mL) were added to a 20 mL microwave tube. The resulting mixture was sparged with N₂ for 5 min and then treated with Pd(dtbpf)Cl₂ (18 mg, 0.027 mmol). The mixture was sparged with N₂ for another 5 min and then heated at 80° C. via microwave irradiation for 1 h. After this time, the reaction vessel was removed from the heating device and allowed to gradually cool to room temperature. The reaction mixture was concentrated to dryness in vacuo to give a brown solid, which was then subjected to silica gel chromatography (0-100% EtOAc/petroleum ether) to give (R)-ethyl 5-((5-cyano-2-methylpyridin-3-yl)amino)-2-(3-(5-(3-hydroxy-1-methyl-2-oxopyrrolidin-3-yl)isoxazol-3-yl)phenyl)thiazole-4-carboxylate as a yellow solid (60 mg, 32%). LCMS (ESI): Mass calcd. for C₂₇H₂₄N₆O₅S 544.15 m/z, found 545.1 [M+1]⁺.

Step D: (R)-5-((5-Cyano-2-methylpyridin-3-yl)amino)-2-(3-(5-(3-hydroxy-1-methyl-2-oxopyrrolidin-3-yl)isoxazol-3-yl)phenyl)thiazole-4-carboxamide. (R)-Ethyl 5-((5-cyano-2-methylpyridin-3-yl)amino)-2-(3-(5-(3-hydroxy-1-methyl-2-oxopyrrolidin-3-yl)isoxazol-3-yl)phenyl)thiazole-4-carboxylate (45 mg, 0.083 mmol) and NH₃ (5.0 mL, 35 mmol, 7 M in MeOH) were added to a 40 mL flask. The resultant mixture was stirred for 16 h at 50° C. After this time, the mixture was concentrated to dryness in vacuo to give a yellow solid, which was subjected to HPLC (Welch Xtimate C18 column, 5 μm, 150×30 mm; 33-63% (v/v) ACN/water (0.05% NH₃H₂O and 10 mM NH₄HCO₃)) to give, after lyophilization, the title compound as a colorless solid (5.3 mg, 12%). LCMS (ESI): Mass calcd. for C₂₅H₂₁N₇O₄S 515.14 m/z, found 516.2 [M+1]⁺. ¹H NMR (400 MHz, DMSO-d₆) δ 11.14 (br s, 1H), 8.60 (d, J=1.4 Hz, 1H), 8.47-8.42 (m, 1H), 8.15 (d, J=1.4 Hz, 1H), 8.13-8.10 (m, 1H), 8.09-8.05 (m, 1H), 8.00-7.94 (m, 1H), 7.84-7.78 (m, 1H), 7.68-7.61 (m, 1H), 7.18 (s, 1H), 6.78 (s, 1H), 3.49-3.46 (m, 2H), 2.85 (s, 3H), 2.64 (s, 3H), 2.58-2.57 (m, 1H), 2.31-2.25 (m, 1H).

### Example 236: (R)-2-(3-(5-(3-Hydroxy-1-methyl-2-oxopyrrolidin-3-yl)isoxazol-3-yl)phenyl)-5-((1-methyl-1H-pyrazol-3-yl)amino)thiazole-4-carboxamide

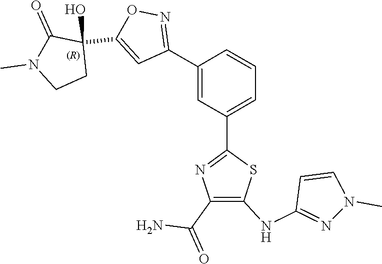

Step A: Ethyl 5-((1-methyl-1H-pyrazol-3-yl)amino)thiazole-4-carboxylate. MeCN (10 mL), DBU (5.1 mL, 34 mmol), ethyl 5-bromothiazole-4-carboxylate (2.0 g, 8.5 mmol), and 1-methyl-1H-pyrazol-3-amine (735 μL, 8.47 mmol) were added to a 20 mL microwave tube. The mixture was heated at 80° C. via microwave irradiation for 1 h. The reaction vessel was removed from the heating device and allowed to gradually cool to rt. The reaction mixture was concentrated to dryness in vacuo to give a brown oil. The oil was then subjected to silica gel chromatography (0-100% EtOAc/petroleum ether) to give ethyl 5-((1-methyl-1H-pyrazol-3-yl)amino)thiazole-4-carboxylate (390 mg, 18% yield) as a green solid. LCMS (ESI): Mass calcd. for C₁₀H₁₂N₄O₂S 252.07 m/z, found 253.1 [M+H]⁺.

Step B: Ethyl 5-((4-bromo-1-methyl-1H-pyrazol-3-yl)amino)thiazole-4-carboxylate. MeCN (15 mL), ethyl 5-((1-methyl-1H-pyrazol-3-yl)amino)thiazole-4-carboxylate (1.2 g, 4.8 mmol) and NBS (847 mg, 4.76 mmol) was added to a 40 mL flask. The resultant mixture was stirred at room temperature for 2 h. After this time, the mixture was treated with sat. aqueous Na₂S₂O₃ (20 mL), and then extracted with EtOAc (30 mL×2). The combined organic extracts were dried over anhydrous Na₂SO₄, filtered, and concentrated to dryness in vacuo to give a brown oil. The oil was then subjected to silica gel chromatography (0-100% EtOAc/petroleum ether) to give ethyl 5-((4-bromo-1-methyl-1H-pyrazol-3-yl)amino)thiazole-4-carboxylate as a brown solid (1.6 g, 83%). LCMS (ESI): Mass calcd. for C₁₀H₁₁BrN₄O₂S 329.98 m/z, found 332.7 [M+H]⁺.

Step C: Ethyl 5-((4-bromo-1-methyl-1H-pyrazol-3-yl)amino)-2-iodothiazole-4-carboxylate. Tetrahydrofuran (20 mL) and ethyl 5-((4-bromo-1-methyl-1H-pyrazol-3-yl)amino)thiazole-4-carboxylate (1.6 g, 4.8 mmol) were added to a nitrogen-purged 100 mL three-necked flask, which was subsequently cooled to −72° C. (dry ice/ethanol) and charged with LDA (2.6 mL, 5.2 mmol, 2.0 M in THF). The resultant mixture was stirred at −72° C. (dry ice/ethanol) for 30 min, treated with 12 (1.47 g, 5.80 mmol) in THF (5 mL) dropwise, and stirred at −72° C. (dry ice/ethanol) for about 20 min. After this time, the mixture was quenched with H₂O (30 mL) and extracted with EtOAc (30 mL×3). The combined organic extracts were dried over anhydrous Na₂SO₄, filtered, and concentrated to dryness in vacuo to give a brown oil, which was subjected to silica gel chromatography (0-100% EtOAc/petroleum ether) to give ethyl 5-((4-bromo-1-methyl-1H-pyrazol-3-yl)amino)-2-iodothiazole-4-carboxylate (1.4 g, 47%) as a pale purple solid. LCMS (ESI): Mass calcd. for C₁₀H₁₀BrIN₄O₂S 455.88 m/z, found 456.9 [M+H]⁺.

Step D: (R)-Ethyl 5-((4-bromo-1-methyl-1H-pyrazol-3-yl)amino)-2-(3-(5-(3-hydroxy-1-methyl-2-oxopyrrolidin-3-yl)isoxazol-3-yl)phenyl)thiazole-4-carboxylate. Ethyl 5-((4-bromo-1-methyl-1H-pyrazol-3-yl)amino)-2-iodothiazole-4-carboxylate (500 mg, 0.806 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 200 mg, 0.521 mmol), K₃PO₄ (513 mg, 2.42 mmol), and 1,4-dioxane/H₂O=4:1 (5 mL) were added to a N₂-purged 20 mL microwave tube, and charged with Pd(dtbpf)Cl₂ (53 mg, 0.081 mmol). The resultant mixture was sparged with N₂ for 5 min, and then heated at 80° C. for 1 hour via microwave irradiation. The reaction mixture was then diluted with H₂O (10 mL) and extracted with EtOAc (10 mL×3). The combined organic extracts were washed with brine (10 mL), dried over anhydrous Na₂SO₄, filtered, and concentrated to dryness in vacuo to give a brown oil. The oil was subjected to silica gel chromatography (0-100% EtOAc/petroleum ether) to give (R)-ethyl 5-((4-bromo-1-methyl-1H-pyrazol-3-yl)amino)-2-(3-(5-(3-hydroxy-1-methyl-2-oxopyrrolidin-3-yl)isoxazol-3-yl)phenyl)thiazole-4-carboxylate as a yellow oil (260 mg, 39%). LCMS (ESI): Mass calcd. for C₂₄H₂₃BrN₆O₅S 586.06 m/z, found 587.0 [M+1]⁺.

Step E: (R)-Ethyl 2-(3-(5-(3-hydroxy-1-methyl-2-oxopyrrolidin-3-yl)isoxazol-3-yl)phenyl)-5-((1-methyl-1H-pyrazol-3-yl)amino)thiazole-4-carboxylate. (R)-Ethyl 5-((4-bromo-1-methyl-1H-pyrazol-3-yl)amino)-2-(3-(5-(3-hydroxy-1-methyl-2-oxopyrrolidin-3-yl)isoxazol-3-yl)phenyl)thiazole-4-carboxylate (260 mg, 0.443 mmol) and THF (10 mL) were added into a 50 mL flask and charged with dry Pd/C (260 mg, 0.244 mmol, 10 wt. %). The reaction vessel was evacuated and refilled with H₂ for three times. The reaction mixture was stirred under H₂ (15 psi) at rt for 16 hours. After this time, the mixture was filtered through a pad of diatomaceous earth, and the pad washed with MeOH (5 mL×2). The filtrate was concentrated in vacuo to afford a colorless oil. The oil was subjected to SFC (DAICEL CHIRALCEL® OD, 10 μm, 250×30 mm; 55% (v/v) EtOH (0.1% NH₃H₂O)/CO₂)) to give (R)-ethyl 2-(3-(5-(3-hydroxy-1-methyl-2-oxopyrrolidin-3-yl)isoxazol-3-yl)phenyl)-5-((1-methyl-1H-pyrazol-3-yl)amino)thiazole-4-carboxylate as a colorless solid (110 mg, 47%). LCMS (ESI): Mass calcd. for C₂₄H₂₄N₆O₅S 508.15 m/z, found 509.2 [M+1]⁺.

Step F: (R)-2-(3-(5-(3-Hydroxy-1-methyl-2-oxopyrrolidin-3-yl)isoxazol-3-yl)phenyl)-5-((1-methyl-1H-pyrazol-3-yl)amino)thiazole-4-carboxamide. NH₃ in MeOH (5 mL, 7 mmol) and (R)-ethyl 2-(3-(5-(3-hydroxy-1-methyl-2-oxopyrrolidin-3-yl)isoxazol-3-yl)phenyl)-5-((1-methyl-1H-pyrazol-3-yl)amino)thiazole-4-carboxylate (110 mg, 0.216 mmol) were added to a 40 mL vial. The resultant mixture was heated to 50° C. and stirred for 3 days. After this time, the mixture was concentrated to dryness in vacuo to afford a colorless oil. The oil was then subjected to SFC (DAICEL CHIRALCEL® OD-H column, 5 m; 250 mm×30 mm; 40% (v/v) EtOH (0.1% NH₃H₂O)/CO₂) to give, after lyophillization, the title compound as a yellow solid (11.3 mg, 10%). LCMS (ESI): Mass calcd. for C₂₂H₂₁N₇O₄S 479.14 m/z, found 480.2 [M+1]⁺. ¹H NMR (400 MHz, DMSO-d₆) δ 10.69 (s, 1H), 8.41-8.36 (m, 1H), 8.05-7.99 (m, 1H), 7.93-7.87 (m, 1H), 7.80 (s, 1H), 7.66 (d, J=2.3 Hz, 1H), 7.64-7.58 (m, 1H), 7.54 (s, 1H), 7.14 (s, 1H), 6.77 (s, 1H), 6.12 (d, J=2.3 Hz, 1H), 3.83 (s, 3H), 3.50-3.45 (m, 2H), 2.85 (s, 3H), 2.61-2.58 (m, 1H), 2.32-2.24 (m, 1H).

### Example 237: (R)-5-(Aminomethyl)-2-(3-(5-(3-hydroxy-1-methyl-2-oxopyrrolidin-3-yl)isoxazol-3-yl)phenyl)thiazole-4-carboxamide

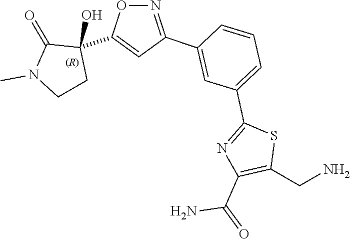

Step A: Methyl 2-bromo-5-((1,3-dioxoisoindolin-2-yl)methyl)thiazole-4-carboxylate. Potassium 1,3-dioxoisoindolin-2-ide (70 mg, 0.378 mmol), and methyl 2-bromo-5-(bromomethyl)thiazole-4-carboxylate (100 mg, 0.317 mmol) in DMF (2 mL) were added to a 8 mL vial. The resulting mixture was stirred at 25° C. for 10 h. After this time, the mixture was diluted with water (20 mL) and extracted with EtOAc (20 mL×3). The combined organic extracts were washed with brine (20 mL), dried over anhydrous Na₂SO₄, filtered, and concentrated to dryness in vacuo to give a yellow oil. The oil was subjected to silica gel chromatography (0-25% EtOAc/petroleum ether) to give methyl 2-bromo-5-((1,3-dioxoisoindolin-2-yl)methyl)thiazole-4-carboxylate as a white solid (80 mg, 61.1% yield). LCMS (ESI): Mass calcd. for C14H9BrN₂O₄S 379.95 m/z, found 380.8 [M+1]⁺.

Step B: (R)-Methyl 5-((1,3-dioxoisoindolin-2-yl)methyl)-2-(3-(5-(3-hydroxy-1-methyl-2-oxopyrrolidin-3-yl)isoxazol-3-yl)phenyl)thiazole-4-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 55 mg, 0.14 mmol), 1,4-dioxane (1.6 mL) and H₂O (0.4 mL) were added to a nitrogen-purged 8 mL flask, and charged with methyl 2-bromo-5-((1,3-dioxoisoindolin-2-yl)methyl)thiazole-4-carboxylate (66 mg, 0.17 mmol), Pd(dtbpf)Cl₂ (10 mg, 0.015 mmol), and K₃PO₄ (93 mg, 0.44 mmol). The mixture was sparged with N₂ for 5 min and then heated at 100° C. for 6 h. After this time, the reaction vessel was removed from the heating device and allowed to gradually cool to rt. The reaction mixture was then diluted with water (10 mL) and extracted with EtOAc (10 mL×3). The combined organic extracts were washed with brine (20 mL), dried over anhydrous Na₂SO₄, filtered, and concentrated to dryness in vacuo to give a yellow oil. The oil was subjected to silica gel chromatography (0-100% EtOAc/petroleum ether) to give (R)-methyl 5-((1,3-dioxoisoindolin-2-yl)methyl)-2-(3-(5-(3-hydroxy-1-methyl-2-oxopyrrolidin-3-yl)isoxazol-3-yl)phenyl)thiazole-4-carboxylate as a white solid (25 mg, 29% yield). LCMS (ESI): Mass calcd. for C₂₈H₂₂N₄O₇S 558.12 m/z, found 559.1 [M+1]⁺.

Step C: (R)-5-(Aminomethyl)-2-(3-(5-(3-hydroxy-1-methyl-2-oxopyrrolidin-3-yl)isoxazol-3-yl)phenyl)thiazole-4-carboxamide. (R)-Methyl 5-((1,3-dioxoisoindolin-2-yl)methyl)-2-(3-(5-(3-hydroxy-1-methyl-2-oxopyrrolidin-3-yl)isoxazol-3-yl)phenyl)thiazole-4-carboxylate (120 mg, 0.215 mmol) and NH₃ (12 mL, 84 mmol, 7 M in MeOH) were added to a 40 mL vial. The resultant mixture was heated at 50° C. for 16 h. After this time, the mixture was concentrated to dryness in vacuo to give a yellow oil. The oil was then subjected to HPLC ((Phenomenex C18 column, 3 m, 75×30 mm), 17-47% ACN/water (0.05% NH₃H₂O+10 mM NH₄HCO₃)), and then subjected to HPLC ((Welch Xtimate C18, 5 m, 150×30 mm) 18-48% ACN/water (0.05% NH₃H₂O+10 mM NH₄HCO₃)) to give the title compound as a white solid (10.71 mg, 12%). LCMS (ESI): Mass calcd. for C₁₉H₁₉N₅O₄S 413.12 m/z found 414.1 [M+H]⁺. ¹H NMR (400 MHz, DMSO-d₆) δ 8.47 (s, 1H), 8.10 (d, J=8.0 Hz, 1H), 7.99 (d, J=8.0 Hz, 1H), 7.93 (br s, 1H), 7.68-7.61 (m, 1H), 7.57 (br s, 1H), 7.17 (s, 1H), 6.74 (s, 1H), 4.25 (s, 2H), 3.53-3.44 (m, 2H), 2.85 (s, 3H), 2.62-2.56 (m, 1H), 2.35-2.23 (m, 1H).

### Example 238: (R)-2-(3-(5-(3-Hydroxy-1-methyl-2-oxopyrrolidin-3-yl)isoxazol-3-yl)phenyl)-5-(((1-methyl-1H-pyrazol-3-yl)amino)methyl)thiazole-4-carboxamide

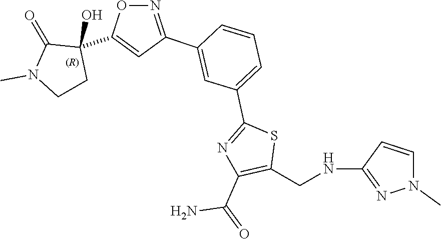

Step A: (R)-Methyl 5-formyl-2-(3-(5-(3-hydroxy-1-methyl-2-oxopyrrolidin-3-yl)isoxazol-3-yl)phenyl)thiazole-4-carboxylate. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 250 mg, 0.651 mmol), methyl 2-bromo-5-formylthiazole-4-carboxylate (325 mg, 1.30 mmol, Example 222 Step B), Pd(dtbpf)Cl₂ (85 mg, 0.13 mmol), K₃PO₄ (552 mg, 2.60 mmol), H₂O (0.5 mL), and 1,4-dioxane (5 mL) were added to a 40 mL vial. The resultant mixture was sparged with N₂ for 5 min, and then heated at 80° C. for 2 h. The reaction vessel was removed from the heating device and allowed to gradually cool to rt. The mixture was filtered through a pad of diatomaceous earth. The filtrate was concentrated to dryness in vacuo to give a brown gum. The gum was subjected to silica gel chromatography (0-5% MeOH/CH₂Cl₂) to give (R)-methyl 5-formyl-2-(3-(5-(3-hydroxy-1-methyl-2-oxopyrrolidin-3-yl)isoxazol-3-yl)phenyl)thiazole-4-carboxylate as a light brown solid (200 mg, 36%). LCMS (ESI): Mass calcd. for C₂₀H₁₇N₃O₆S 427.08 m/z, found 428.1 [M+H]⁺.

Step B: (R)-Methyl 2-(3-(5-(3-hydroxy-1-methyl-2-oxopyrrolidin-3-yl)isoxazol-3-yl)phenyl)-5-(((1-methyl-1H-pyrazol-3-yl)amino)methyl)thiazole-4-carboxylate. (R)-Methyl 5-formyl-2-(3-(5-(3-hydroxy-1-methyl-2-oxopyrrolidin-3-yl)isoxazol-3-yl)phenyl)thiazole-4-carboxylate (200 mg, 0.468 mmol), 1-methyl-1H-pyrazol-3-amine (125 mg, 0.936 mmol), titanium(IV) propan-2-olate (199 mg, 0.702 mmol), and MeOH (8 mL) were added to a 40 mL vial. The vial was capped, placed on a stir plate, and stirred for 2 h at room temperature. NaBH₃CN (73.5 mg, 1.17 mmol) was added in one portion, and the resulting mixture heated to 60° C. and stirred for 16 h. The reaction vessel was removed from the heating device and allowed to gradually cool to room temperature. The reaction mixture was used for the next step directly without workup and purification. LCMS (ESI): Mass calcd. for C₂₄H₂₄N₆O₅S 508.15 m/z, found 509.3 [M+H]⁺.

Step C: (R)-2-(3-(5-(3-Hydroxy-1-methyl-2-oxopyrrolidin-3-yl)isoxazol-3-yl)phenyl)-5-(((1-methyl-1H-pyrazol-3-yl)amino)methyl)thiazole-4-carboxamide. To (R)-methyl 2-(3-(5-(3-hydroxy-1-methyl-2-oxopyrrolidin-3-yl)isoxazol-3-yl)phenyl)-5-(((1-methyl-1H-pyrazol-3-yl)amino)methyl)thiazole-4-carboxylate (238 mg, 0.468 mmol) in MeOH (8 mL), NH₃ (8 mL, 56 mmol, 7 M in MeOH) was added and the resulting mixture stirred for 18 h at 75° C. The reaction vessel was removed from the heating device and allowed to gradually cool to rt. The mixture was concentrated in vacuo to give a brown gum, which was subjected to HPLC (Boston Prime C18 column, 5 μm, 150×30 mm; 25-55% CH₃CN/H₂O with 0.05% NH₃H₂O+10 mM NH₄HCO₃), and then subjected to SFC (DAICEL CHIRALCEL® AS column, 10 μm, 250×30 mm; 55% EtOH (containing 0.1% NH₃·H₂O)/supercritical CO₂) to give, after lyophilization, (R)-2-(3-(5-(3-hydroxy-1-methyl-2-oxopyrrolidin-3-yl)isoxazol-3-yl)phenyl)-5-(((1-methyl-1H-pyrazol-3-yl)amino)methyl)thiazole-4-carboxamide as a white solid (49.9 mg, 66%). LCMS (ESI): Mass calcd. for C₂₃H₂₃N₇O₄S 493.15 m/z, found 494.2 [M+H]⁺. ¹H NMR (400 MHz, DMSO-d₆) δ 8.44 (s, 1H), 8.07 (d, J=8.0 Hz, 1H), 8.03 (br s, 1H), 7.98 (d, J=8.0 Hz, 1H), 7.66 (br s, 1H), 7.65-7.59 (m, 1H), 7.34 (d, J=2.0 Hz, 1H), 7.17 (s, 1H), 6.76 (br s, 1H), 6.10-6.02 (m, 1H), 5.47 (d, J=2.0 Hz, 1H), 4.79 (d, J=6.4 Hz, 2H), 3.60 (s, 3H), 3.52-3.43 (m, 2H), 2.84 (s, 3H), 2.61-2.54 (m, 1H), 2.34-2.22 (m, 1H). ¹H NMR (400 MHz, CD₃OD) δ 8.45 (s, 1H), 8.04 (d, J=8.0 Hz, 1H), 7.94 (d, J=8.0 Hz, 1H), 7.64-7.55 (m, 1H), 7.29 (d, J=2.4 Hz, 1H), 7.01 (s, 1H), 5.61 (d, J=2.4 Hz, 1H), 4.91-4.90 (m, 2H), 3.69 (s, 3H), 3.63-3.56 (m, 2H), 2.99 (s, 3H), 2.80-2.70 (m, 1H), 2.47-2.37 (m, 1H).

### Example 239: (R)-5-((1,4-Dimethyl-1H-pyrazol-3-yl)amino)-2-(3-(5-(3-hydroxy-1-methyl-2-oxopyrrolidin-3-yl)isoxazol-3-yl)phenyl)thiazole-4-carboxamide

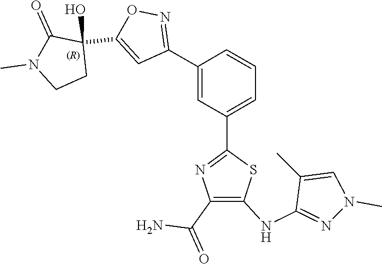

Step A: Ethyl 5-((1,4-dimethyl-1H-pyrazol-3-yl)amino)thiazole-4-carboxylate. Ethyl 5-bromo-1,3-thiazole-4-carboxylate (1.0 g, 4.2 mmol), 1,4-dimethylpyrazol-3-amine (471 mg, 4.24 mmol), Cs₂CO₃ (3.45 g, 10.6 mmol), XPhos (735 mg, 1.27 mmol), 1,4-dioxane (10 mL), and Pd(dba)₂ (244 mg, 0.424 mmol) were added to a 100 mL round-bottomed flask, which was evacuated and refilled with N₂ for three times. The resultant mixture was heated at 100° C. for 8 h. After this time, the reaction vessel was removed from the oil bath and allowed to gradually cool to rt. The reaction mixture was diluted with water (80 mL) and extracted with DCM (80 mL×2). The combined organic extracts were dried over anhydrous Na₂SO₄, filtered, and concentrated to dryness in vacuo. The residue was subjected to silica gel chromatography (1-3% EtOAc/petroleum ether) to give ethyl 5-((1,4-dimethyl-1H-pyrazol-3-yl)amino)thiazole-4-carboxylate as a yellow solid (721 mg, 64%). ¹H NMR (400 MHz, CDCl₃) δ 9.88 (br s, 1H), 8.06 (s, 1H), 7.08 (s, 1H), 4.46 (q, J=7.2 Hz, 2H), 3.81 (s, 3H), 2.07 (s, 3H), 1.47 (t, J=7.2 Hz, 3H).

Step B: Ethyl 2-bromo-5-((1,4-dimethyl-1H-pyrazol-3-yl)amino)thiazole-4-carboxylate. NBS (529 mg, 2.97 mmol) was added to a solution consisting of ethyl 5-((1,4-dimethyl-1H-pyrazol-3-yl)amino)thiazole-4-carboxylate (720 mg, 2.70 mmol) in MeCN (10 mL). The resultant mixture was stirred at rt for 1 h. After this time, the mixture was diluted with water (50 mL) and extracted with DCM (50 mL×2). The combined organic extracts were dried over anhydrous Na₂SO₄, filtered, and concentrated to dryness in vacuo. The residue was subjected to silica gel chromatography (1-3% EtOAc/petroleum ether) to afford ethyl 2-bromo-5-((1,4-dimethyl-1H-pyrazol-3-yl)amino)thiazole-4-carboxylate as a yellow solid (421 mg, 45%). ¹H NMR (400 MHz, chloroform-d) δ 9.97 (br s, 1H), 7.08 (s, 1H), 4.44 (q, J=7.2 Hz, 2H), 3.80 (s, 3H), 2.06 (s, 3H), 1.44 (t, J=7.1 Hz, 3H).

Step C: (R)-Ethyl 5-((1,4-dimethyl-1H-pyrazol-3-yl)amino)-2-(3-(5-(3-hydroxy-1-methyl-2-oxopyrrolidin-3-yl)isoxazol-3-yl)phenyl)thiazole-4-carboxylate. Ethyl 2-bromo-5-((1,4-dimethyl-1H-pyrazol-3-yl)amino)thiazole-4-carboxylate (150 mg, 0.434 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 4, 184 mg, 0.478 mmol), 1,4-dioxane (6 mL), H₂O (0.5 mL), Pd(PPh₃)₄ (50 mg, 0.044 mmol), and Na₂CO₃ (138 mg, 1.3 mmol) were added to a 10 mL microwave tube. The mixture was sparged with N₂ for 5 minutes and heated at 110° C. for 1 h via microwave irradiation. After this time, the reaction vessel was removed from the heating device and allowed to gradually cool to rt. The reaction mixture was diluted with water (30 mL) and extracted with DCM (30 mL×2). The combined organic extracts were dried over anhydrous Na₂SO₄, filtered, and concentrated. The residue was subjected to silica gel chromatography (1-5% MeOH/DCM) to afford the desired product as a red solid (253 mg, 67%). LCMS (ESI): Mass calcd. for C₂₅H₂₆N₆O₅S 522.17 m/z, found 523.2 [M+H]⁺.

Step D: (R)-5-((1,4-Dimethyl-1H-pyrazol-3-yl)amino)-2-(3-(5-(3-hydroxy-1-methyl-2-oxopyrrolidin-3-yl)isoxazol-3-yl)phenyl)thiazole-4-carboxamide. (R)-Ethyl 5-((1,4-dimethyl-1H-pyrazol-3-yl)amino)-2-(3-(5-(3-hydroxy-1-methyl-2-oxopyrrolidin-3-yl)isoxazol-3-yl)phenyl)thiazole-4-carboxylate (200 mg, 0.383 mmol) and NH₃ (6 mL, 7 M in MeOH, 42 mmol) were added to a 10 mL microwave tube. The resultant mixture was heated at 125° C. via microwave irradiation for 5 h. After this time, the reaction vessel was removed from the heating device and allowed to gradually cool to rt. The reaction mixture was concentrated in vacuo. The residue was subjected to HPLC (Boston Green ODS column, 150×30 mm, 5 μm, 35-95% MeCN/H₂O (0.075% TFA)) to give the title compound as a yellow solid (11.29 mg, 6%). LCMS (ESI): Mass calcd. for C₂₃H₂₃N₇O₄S 493.15 m/z, found 494.2 [M+H]⁺. ¹H NMR (400 MHz, DMSO-d₆) δ 10.80 (s, 1H), 8.38 (s, 1H), 8.04-7.99 (m, 1H), 7.93-7.88 (m, 1H), 7.80 (s, 1H), 7.64-7.58 (m, 1H), 7.55 (s, 1H), 7.49 (s, 1H), 7.14 (s, 1H), 6.76 (s, 1H), 3.78 (s, 3H), 3.53-3.40 (m, 2H), 2.85 (s, 3H), 2.62-2.55 (m, 1H), 2.35-2.26 (m, 1H), 1.99 (s, 3H). ¹H NMR (400 MHz, MeOH-d₄) δ 8.37 (s, 1H), 7.99-7.98 (m, 1H), 7.85-7.83 (m, 1H), 7.57-7.54 (m, 1H), 7.28 (s, 1H), 7.00 (s, 1H), 3.81 (s, 3H), 3.64-3.54 (m, 2H), 2.98 (s, 3H), 2.82-2.71 (m, 1H), 2.47-2.37 (m, 1H), 2.04 (s, 3H).

### Example 240: (R)-6-(2-Fluoro-5-(5-(3-hydroxy-1-methyl-2-oxopyrrolidin-3-yl)isoxazol-3-yl)phenyl)-4-(trifluoromethyl)picolinamide

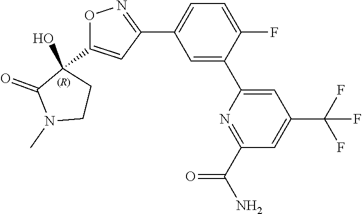

Step A: (E)-3-Bromo-4-fluorobenzaldehyde oxime. Na₂CO₃ (428 mg, 4.04 mmol) was added to a mixture consisting of 3-bromo-4-fluorobenzaldehyde (1 g, 4.9 mmol), hydroxylamine hydrochloride (550 mg, 7.92 mmol), EtOH (20 mL), and H₂O (10 mL), and the mixture stirred at room temperature for 18 h. After this time, the mixture was concentrated in vacuo to remove EtOH and the aqueous extracted with EtOAc (25 mL×3). The combined organic layer was washed with water (25 mL×3) and brine (25 mL×2), dried over anhydrous Na₂SO₄, filtered, and concentrated in vacuo to afford (E)-3-bromo-4-fluorobenzaldehyde oxime as a white solid (1 g, 93%). MS (ESI): Mass calcd. for C₇H₅BrFNO 216.95 m/z, found 218.0 [M+H]⁺.

Step B: (Z)-3-Bromo-4-fluoro-N-hydroxybenzimidoyl chloride. To a solution consisting of (E)-3-bromo-4-fluorobenzaldehyde oxime (1 g, 4.6 mmol) and CH₂Cl₂ (25 mL) was added NCS (643 mg, 4.82 mmol) in one portion. The resultant mixture was stirred at room temperature for 18 h. After this time, the solution was concentrated in vacuo, and the residue subjected to silica gel chromatography (0-30% EtOAc/petroleum ether) to afford (Z)-3-bromo-4-fluoro-N-hydroxybenzimidoyl chloride as a pale yellow solid (620 mg, 54%). MS (ESI): Mass calcd. for C₇H₄BrClFNO 250.91 m/z, found 251.9 [M+H]⁺.

Step C: (R)-3-(3-(3-Bromo-4-fluorophenyl)isoxazol-5-yl)-3-hydroxy-1-methylpyrrolidin-2-one. (Z)-3-Bromo-4-fluoro-N-hydroxybenzimidoyl chloride (567 mg, 2.25 mmol) was added to a mixture consisting of (R)-3-ethynyl-3-hydroxy-1-methylpyrrolidin-2-one (Intermediate 1, 250 mg, 1.8 mmol), NaHCO₃ (604 mg, 7.2 mmol), sodium ascorbate (107 mg, 0.54 mmol), Cu₂SO₄₅H₂O (45 mg, 0.18 mmol), THF (10 mL) and H₂O (10 mL). The resulting mixture was stirred at room temperature for 18 h. After this time, the mixture was diluted with water (10 mL) and extracted with EtOAc (25 mL×3). The combined organic extracts were washed with water (10 mL×2) and brine (10 mL×2), dried over anhydrous Na₂SO₄, filtered, and concentrated in vacuo.

The residue was subjected to silica gel chromatography (0-100% EtOAc/petroleum ether) to afford (R)-3-(3-(3-bromo-4-fluorophenyl)isoxazol-5-yl)-3-hydroxy-1-methylpyrrolidin-2-one as an off-white solid (550 mg, 86%). MS (ESI): Mass calcd. for C₁₄H₁₂BrFN₂O₃ 354.00 m/z, found 355.0 [M+H]⁺.

Step D: (R)-(2-Fluoro-5-(5-(3-hydroxy-1-methyl-2-oxopyrrolidin-3-yl)isoxazol-3-yl)phenyl)boronic acid. A mixture consisting of (R)-3-(3-(3-bromo-4-fluorophenyl)isoxazol-5-yl)-3-hydroxy-1-methylpyrrolidin-2-one (200 mg, 0.56 mmol), (BPin)₂ (286 mg, 1.13 mmol), KOAc (166 mg, 1.69 mmol), and anhydrous 1,4-dioxane (5 mL) was sparged with N₂ for 5 min, treated with X-Phos-Pd-G2 (44.3 mg, 0.056 mmol), and heated at 100° C. for 18 h under a N₂ atmosphere. After this time, the reaction vessel was removed from the oil bath and allowed to gradually cool to room temperature. The reaction mixture was used to next step directly without work-up and purification. MS (ESI): Mass calcd. for C₁₄H₁₄BFN₂O₅ 320.08 m/z, found 321.1 [M+H]⁺.

Step E: (R)-6-(2-Fluoro-5-(5-(3-hydroxy-1-methyl-2-oxopyrrolidin-3-yl)isoxazol-3-yl)phenyl)-4-(trifluoromethyl)picolinamide. A mixture consisting of (R)-(2-fluoro-5-(5-(3-hydroxy-1-methyl-2-oxopyrrolidin-3-yl)isoxazol-3-yl)phenyl)boronic acid (180 mg, 0.56 mmol, in 5 mL of 1,4-dioxane), 6-chloro-4-(trifluoromethyl)picolinamide (126 mg, 0.56 mmol), K₃PO₄ (230 mg, 1.69 mmol) and water (0.5 mL) was sparged with N₂ for 5 min, treated with Pd(dtbpf)Cl₂ (37 mg, 0.056 mmol), and heated at 100° C. for 20 h. The reaction vessel was removed from the oil bath and allowed to gradually cool to rt. The mixture was filtered through a pad of diatomaceous earth, and the pad washed with EtOAc (15 mL). The filtrate was concentrated in vacuo, and the residue subjected to HPLC (Boston Green ODS column, 5 μm, 150×30 mm; 38-68% (v/v) CH₃CN/H₂O with 0.225% FA), and then subjected to SFC (DAICEL CHIRALCEL® OD-H column, 5 μm, 250×30 mm; 35% EtOH (containing 0.1% NH₃H₂O)/supercritical CO₂) to give, after lyophilization, (R)-6-(2-fluoro-5-(5-(3-hydroxy-1-methyl-2-oxopyrrolidin-3-yl)isoxazol-3-yl)phenyl)-4-(trifluoromethyl)picolinamide as a white solid (46.6 mg, 20%). MS (ESI): Mass calcd. for C₂₁H₁₆F₄N₄O₄ 464.11 m/z, found 465.1 [M+H]⁺. ¹H NMR (400 MHz, DMSO-d₆) δ 8.64-8.57 (m, 1H), 8.54 (s, 1H), 8.36 (s, 1H), 8.28 (s, 1H), 8.14-8.08 (m, 1H), 8.03 (s, 1H), 7.61-7.54 (m, 1H), 7.23 (s, 1H), 6.77 (br s, 1H), 3.52-3.43 (m, 2H), 2.85 (s, 3H), 2.60-2.53 (m, 1H), 2.33-2.24 (m, 1H). ¹H NMR (400 MHz, CD₃OD) δ 8.70 (dd, J=2.4, 7.6 Hz, 1H), 8.39 (s, 1H), 8.35 (s, 1H), 8.09-8.02 (m, 1H), 7.52-7.43 (m, 1H), 7.07 (s, 1H), 3.64-3.56 (m, 2H), 2.99 (s, 3H), 2.81-2.72 (m, 1H), 2.48-2.37 (m, 1H).

### Example 241: (R)-5-Amino-4-cyclopropyl-4′-(5-(3-hydroxy-1-methyl-2-oxopyrrolidin-3-yl)isoxazol-3-yl)-[2,2′-bipyridine]-6-carboxamide

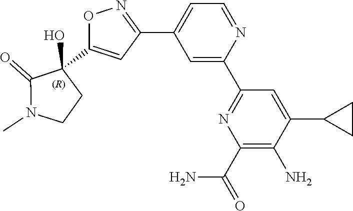

Step A: 3-Amino-6-chloro-4-cyclopropylpicolinonitrile. Cyclopropylboronic acid (2.80 g, 32.6 mmol) was added into a solution of 3-amino-4-bromo-6-chloropicolinonitrile (5.00 g, 21.5 mmol) and Cs₂CO₃ (21.0 g, 64.5 mmol) in 1,4-dioxane (100 mL) and H₂O (25 mL). The resultant mixture was sparged with N₂ for 5 min, treated with Pd(dppf)Cl₂ (2.35 g, 3.21 mmol), and stirred while heating at 100° C. for 16 hours. The reaction vessel was removed from the oil bath and allowed to gradually cool to rt. The reaction mixture was diluted with H₂O (100 mL) and extracted with ethyl acetate (120 mL×3). The combined organic extracts were washed with brine, dried with anhydrous Na₂SO₄, filtered, and concentrated to dryness in vacuo and the residue subjected to silica gel chromatography (0-100% EtOAc/petroleum ether) to afford 3-amino-6-chloro-4-cyclopropylpicolinonitrile (1.95 g, 44%) as a yellow solid. MS (ESI): Mass calcd. for C₉H₈ClN₃ 193.04 m/z, found 194.0 [M+H]⁺.

Step B: 3-Amino-6-chloro-4-cyclopropylpicolinamide. H₂O₂ (4.10 mL, 40.9 mmol) was added to a mixture consisting of 3-amino-6-chloro-4-cyclopropylpicolinonitrile (1.95 g, 10.1 mmol), K₂CO₃ (683 mg, 4.94 mmol) and DMF (20 mL). The resultant mixture was stirred at 30° C. for 24 h. After this time, the mixture was diluted with saturated aqueous Na₂SO₃ (30 mL) and extracted with ethyl acetate (25 mL×3). The combined organic extracts were washed with 4% LiCl solution (30 mL), dried over anhydrous Na₂SO₄, filtered, and concentrated to dryness in vacuo to give 3-amino-6-chloro-4-cyclopropylpicolinamide as a yellow solid (1.88 g, 80%). MS (ESI): Mass calcd. for C₉H₁₀ClN₃O 211.05 m/z, found 211.8 [M+H]⁺.

Step C: 3-Amino-4-cyclopropyl-6-(tributylstannyl)picolinamide. 1,1,1,2,2,2-Hexabutyldistannane (2.35 g, 4.05 mmol) was added into a mixture consisting of 3-amino-6-chloro-4-cyclopropylpicolinamide (500 mg, 2.36 mmol), LiCl (600 mg, 14.2 mmol) and 1,4-dioxane (30 mL). The resultant mixture was sparged with N₂ for 5 min, treated with Pd₂(dba)₃ (110 mg, 0.120 mmol) and tricyclohexylphosphine (70 mg, 0.25 mmol), and stirred while heating at 120° C. for 16 h. The reaction vessel was removed from the oil bath and allowed to gradually cool to rt. The mixture was quenched with sat. aqueous KF (30 mL) and extracted with ethyl acetate (50 mL×3). The combined organic extracts were washed with brine (40 mL), dried over anhydrous Na₂SO₄, filtered, concentrated to dryness in vacuo, and the residue subjected to HPLC (Xtimate C18 column, 5 μm, 150×40 mm, 95-99% (v/v) CH₃CN/H₂O with 0.05% NH₃H₂O) to give 3-amino-4-cyclopropyl-6-(tributylstannyl)picolinamide as a brown oil (315 mg, 27%). MS (ESI): Mass calcd. for C₂₁H₃₇N₃OSn 467.20 m/z, found 468.2 [M+H]⁺.

Step D: (R)-5-Amino-4-cyclopropyl-4′-(5-(3-hydroxy-1-methyl-2-oxopyrrolidin-3-yl)isoxazol-3-yl)-[2,2′-bipyridine]-6-carboxamide. 3-Amino-4-cyclopropyl-6-(tributylstannyl)picolinamide (240 mg, 0.515 mmol) was added into a solution consisting of (R)-3-(3-(2-chloropyridin-4-yl)isoxazol-5-yl)-3-hydroxy-1-methylpyrrolidin-2-one (Intermediate 65, 140 mg, 0.477 mmol), TEA (140 μL, 1.00 mmol) and toluene (15 mL). The resultant mixture was sparged with N₂ for 5 min, treated with Pd(PPh₃)₄ (55 mg, 0.048 mmol) and stirred while heating at 120° C. for 16 h. The reaction vessel was removed from the oil bath and allowed to gradually cool to rt. The reaction mixture was quenched with sat. aqueous KF (30 mL) and extracted with EtOAc (20 mL×3). The combined organic extracts were washed with brine, dried over anhydrous Na₂SO₄, filtered, and concentrated to dryness in vacuo. The residue was subjected to HPLC (Phenomenex Gemini-NX C18 column, 3 μm, 75×30 mm; 27-57% (v/v) CH₃CN/H₂O with 0.05% NH₃H₂O+10 mM NH₄HCO₃) to give (R)-5-amino-4-cyclopropyl-4′-(5-(3-hydroxy-1-methyl-2-oxopyrrolidin-3-yl)isoxazol-3-yl)-[2,2′-bipyridine]-6-carboxamide as a white solid (45.7 mg, 22%). MS (ESI): Mass calcd. for C₂₂H₂₂N₆O₄ 434.17 m/z found 434.4 [M+H]⁺. ¹H NMR (400 MHz, DMSO-d₆) δ 8.79 (s, 1H), 8.71 (d, J=5.1 Hz, 1H), 8.51 (s, 1H), 8.05 (s, 1H), 7.80-7.75 (m, 1H), 7.53 (s, 1H), 7.45 (s, 1H), 7.31 (br s, 2H), 6.79 (s, 1H), 3.51-3.42 (m, 2H), 2.85 (s, 3H), 2.61-2.55 (m, 1H), 2.31-2.25 (m, 1H), 1.92-1.83 (m, 1H), 1.08-1.03 (m, 2H), 0.70-0.66 (m, 2H).

### Example 242: (R)-3-Amino-4-cyclopropyl-6-(2-fluoro-3-(5-(3-hydroxy-1-methyl-2-oxopyrrolidin-3-yl)isoxazol-3-yl)phenyl)picolinamide

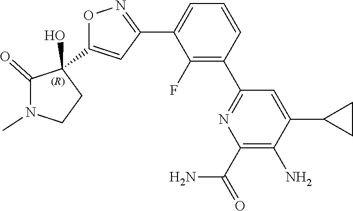

Step A: (E)-3-Bromo-2-fluorobenzaldehyde oxime. A mixture consisting of 3-bromo-2-fluorobenzaldehyde (5.00 g, 24.6 mmol), hydroxylamine hydrochloride (2.05 g, 29.6 mmol), NaOAc (2.43 g, 29.6 mmol) and EtOH (70 mL) was heated at 45° C. for 1 hour. After this time, the mixture was concentrated to dryness in vacuo, and the residue suspended in H₂O (100 mL) and extracted with EtOAc (100 mL×3). The combined organic layers were washed with brine (100 mL), dried over anhydrous Na₂SO₄, filtered, and concentrated to dryness in vacuo to afford (E)-3-bromo-2-fluorobenzaldehyde oxime as a white solid (5.4 g, 99%). MS (ESI): Mass calcd. for C₇H₅BrFNO 216.95 m/z, found 217.9 [M+H]⁺. ¹H NMR (400 MHz, DMSO-d₆) δ 11.77 (s, 1H), 8.23 (s, 1H), 7.76-7.70 (m, 2H), 7.19 (t, J=7.9 Hz, 1H).

Step B: (Z)-3-Bromo-2-fluoro-N-hydroxybenzimidoyl chloride. NCS (1.35 g, 10.1 mmol) was added to a solution consisting of (E)-3-bromo-2-fluorobenzaldehyde oxime (2.00 g, 9.17 mmol) in DMF (20 mL). The resultant mixture was stirred at room temperature for 2 h, diluted with H₂O (30 mL), and extracted with MTBE (30 mL×3). The combined organic extracts were washed with 4% aqueous LiCl solution (100 mL) and brine (30 mL), dried over anhydrous Na₂SO₄, filtered, and concentrated to dryness in vacuo to give (Z)-3-bromo-2-fluoro-N-hydroxybenzimidoyl chloride as a white solid (2.3 g, 72%). MS (ESI): Mass calcd. for C₇H₄BrClFNO 250.91 m/z, found 251.9 [M+H]⁺.

Step C: (R)-3-(3-(3-Bromo-2-fluorophenyl)isoxazol-5-yl)-3-hydroxy-1-methylpyrrolidin-2-one. (R)-3-Ethynyl-3-hydroxy-1-methylpyrrolidin-2-one (Intermediate 1, 441 mg, 3.17 mmol) was added to a mixture consisting of (Z)-3-bromo-2-fluoro-N-hydroxybenzimidoyl chloride (800 mg, 3.17 mmol), TEA (1.32 mL, 9.5 mmol), and DCM (7 mL). The resultant mixture was stirred for 16 h at room temperature, treated with H₂O (10 mL), and extracted with DCM (10 mL×3). The combined organic extracts were washed with brine (10 mL), dried over anhydrous Na₂SO₄, filtered, and concentrated to dryness in vacuo. The residue was subjected to silica gel chromatography (0-25% EtOAc/petroleum ether) to give (R)-3-(3-(3-bromo-2-fluorophenyl)isoxazol-5-yl)-3-hydroxy-1-methylpyrrolidin-2-one as a white solid (677 mg, 58%). MS (ESI): Mass calcd. for C₁₄H₁₂BrFN₂O₃ 354.00 m/z, found 355.0 [M+H]⁺. ¹H NMR (400 MHz, DMSO-d₆) δ 7.92-7.85 (m, 2H), 7.32 (t, J=7.7 Hz, 1H), 6.90 (d, J=3.1 Hz, 1H), 6.79 (s, 1H), 3.51-3.38 (m, 2H), 2.83 (s, 3H), 2.61-2.53 (m, 1H), 2.32-2.22 (m, 1H).

Step D: (R)-3-(3-(2-Fluoro-3-(4,4,5,5-tetramethyl-1,3,2-dioxaborolan-2-yl)phenyl)isoxazol-5-yl)-3-hydroxy-1-methylpyrrolidin-2-one. (R)-3-(3-(3-Bromo-2-fluorophenyl)isoxazol-5-yl)-3-hydroxy-1-methylpyrrolidin-2-one (390 mg, 1.10 mmol), 4,4,4′,4′,5,5,5′,5′-octamethyl-2,2′-bi(1,3,2-dioxaborolane) (418 mg, 1.65 mmol) and KOAc (323 mg, 3.29 mmol) were added to a 10 mL flask and the resulting mixture dissolved in 1,4-dioxane (5 mL). The resultant mixture was sparged with N₂ for 5 min, treated with Pd(dppf)Cl₂ (80 mg, 0.11 mmol), and heated at 100° C. for 3 hour. The reaction vessel was removed from the oil bath and allowed to gradually cool to rt. The reaction mixture was concentrated to dryness in vacuo, and the residue subjected to silica gel chromatography (0-100% EtOAc/petroleum ether) to give (R)-3-(3-(2-fluoro-3-(4,4,5,5-tetramethyl-1,3,2-dioxaborolan-2-yl)phenyl)isoxazol-5-yl)-3-hydroxy-1-methylpyrrolidin-2-one as a brown solid (190 mg, 35%). MS (ESI): Mass calcd. for C₂₀H₂₄BFN₂O₅ 402.18 m/z, found 403.2 [M+H]⁺.

Step E: (R)-3-Amino-4-cyclopropyl-6-(2-fluoro-3-(5-(3-hydroxy-1-methyl-2-oxopyrrolidin-3-yl)isoxazol-3-yl)phenyl)picolinamide. (R)-3-(3-(2-Fluoro-3-(4,4,5,5-tetramethyl-1,3,2-dioxaborolan-2-yl)phenyl)isoxazol-5-yl)-3-hydroxy-1-methylpyrrolidin-2-one (240 mg, 0.597 mmol), 3-amino-6-chloro-4-cyclopropylpicolinamide (126 mg, 0.597 mmol, Example 88 Step A), and K₃PO₄ (380 mg, 1.79 mmol) were added to a 5 mL microwave tube and the resulting mixture dissolved in 1,4-dioxane (4 mL) and H₂O (1 mL). The mixture was sparged with N₂ for 5 min, treated with Pd(dtbpf)Cl₂ (39 mg, 0.060 mmol), and heated at 90° C. via microwave irradiation for 1 h. The reaction vessel was removed from the microwave heating device and allowed to gradually cool to rt. The reaction mixture was concentrated to dryness in vacuo, and the residue subjected to HPLC (Boston Prime C18 column, 5 μm, 150×30 mm; 35-65% (v/v) ACN/H₂O with 0.05% NH₃H₂O and 10 mM NH₄HCO₃) to give the title compound as a colorless solid (80.1 mg, 30%). MS (ESI): Mass calcd. for C₂₃H₂₂FN₅O₄ 451.17 m/z, found 452.1 [M+1]⁺. ¹H NMR (400 MHz, DMSO-d₆) δ 8.22-8.15 (m, 1H), 8.14-8.07 (m, 1H), 7.86-7.78 (m, 1H), 7.52-7.44 (m, 2H), 7.43-7.36 (m, 1H), 7.16 (br s, 2H), 6.93 (d, J=3.1 Hz, 1H), 6.77 (s, 1H), 3.53-3.44 (m, 2H), 2.85 (s, 3H), 2.63-2.55 (m, 1H), 2.35-2.24 (m, 1H), 1.92-1.81 (m, 1H), 1.08-0.99 (m, 2H), 0.73-0.65 (m, 2H).

### Example 243: (R)-3-Amino-4-cyclopropyl-6-(3-fluoro-5-(5-(3-hydroxy-1-methyl-2-oxopyrrolidin-3-yl)isoxazol-3-yl)phenyl)picolinamide

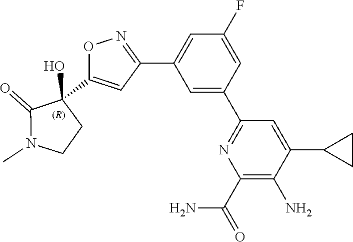

Step A: (E)-3-Bromo-5-fluorobenzaldehyde oxime. To a mixture consisting of 3-bromo-5-fluorobenzaldehyde (2.0 g, 9.9 mmol), NH₂OH·HCl (1.1 g, 16 mmol), EtOH (40 mL) and H₂O (20 mL) was added Na₂CO₃ (856 mg, 8.08 mmol) in one portion. The resulting mixture was stirred for 16 h at room temperature. After this time, the mixture was concentrated in vacuo to remove the volatiles, diluted with 10 mL of H₂O, and extracted with EtOAc (30 mL×2). The combined organic extracts were washed with water (30 mL×3) and brine (30 mL×2), dried over anhydrous Na₂SO₄, filtered, and concentrated to dryness in vacuo to give (E)-3-bromo-5-fluorobenzaldehyde oxime as a white solid (2.14 g, 99.6%). MS (ESI): Mass calcd. for C₇HsBrFNO 216.95 m/z, found 217.9 [M+H]⁺.

Step B: (Z)-3-Bromo-5-fluoro-N-hydroxybenzimidoyl chloride. To a solution of (E)-3-bromo-5-fluorobenzaldehyde oxime (400 mg, 1.84 mmol) in MeCN (4 mL) was added NCS (269 mg, 2.02 mmol) in one portion. The resulting mixture was stirred for 6.5 h at 0° C. After this time, the reaction mixture containing (Z)-3-bromo-5-fluoro-N-hydroxybenzimidoyl chloride was used for next step directly without purification.

Step C: (R)-3-(3-(3-Bromo-5-fluorophenyl)isoxazol-5-yl)-3-hydroxy-1-methylpyrrolidin-2-one. Sodium ascorbate (85 mg, 0.43 mmol) was added to a mixture consisting of (Z)-3-bromo-5-fluoro-N-hydroxybenzimidoyl chloride (453 mg, 1.80 mmol), (R)-3-ethynyl-3-hydroxy-1-methylpyrrolidin-2-one (Intermediate 1, 200 mg, 1.44 mmol), NaHCO₃ (483 mg, 5.75 mmol), Cu₂SO₄·5H₂O (36 mg, 0.14 mmol), MeCN (4 mL), and H₂O (2.5 mL). The resulting mixture was stirred for 18 h at room temperature. After this time, the mixture was diluted with H₂O (20 mL) and extracted with EtOAc (20 mL×2). The combined organic extracts were washed with brine (20 mL), dried over anhydrous Na₂SO₄, filtered, and concentrated to dryness in vacuo. The residue was purified by silica gel chromatography (0-60% EtOAc/petroleum ether) to afford (R)-3-(3-(3-bromo-5-fluorophenyl)isoxazol-5-yl)-3-hydroxy-1-methylpyrrolidin-2-one as a white solid (380 mg, 74%). ¹H NMR (400 MHz, CDCl₃) 7.65 (s, 1H), 7.41-7.36 (m, 1H), 7.33-7.29 (m, 1H), 6.64 (s, 1H), 4.45 (s, 1H), 3.71-3.59 (m, 1H), 3.57-3.47 (m, 1H), 3.01 (s, 3H), 2.76-2.72 (m, 1H), 2.52-2.42 (m, 1H).

Step D: (R)-3-(3-(3-Fluoro-5-(4,4,5,5-tetramethyl-1,3,2-dioxaborolan-2-yl)phenyl)isoxazol-5-yl)-3-hydroxy-1-methylpyrrolidin-2-one. A mixture consisting of (R)-3-(3-(3-bromo-5-fluorophenyl)isoxazol-5-yl)-3-hydroxy-1-methylpyrrolidin-2-one (300 mg, 0.85 mmol), (BPin)₂ (429 mg, 1.7 mmol), KOAc (207 mg, 2.11 mmol), and anhydrous 1,4-dioxane (10 mL) was sparged with N₂ for 5 min, treated with Pd(dppf)Cl₂·CH₂Cl₂ (69 mg, 0.084 mmol), and heated at 100° C. for 3 h under a N₂ atmosphere. After this time the reaction vessel was removed from the oil bath and allowed to gradually cool to rt. The reaction mixture containing (R)-3-(3-(3-fluoro-5-(4,4,5,5-tetramethyl-1,3,2-dioxaborolan-2-yl)phenyl)isoxazol-5-yl)-3-hydroxy-1-methylpyrrolidin-2-one (340 mg) was used for next step directly without workup and purification. MS (ESI): Mass calcd. for C₂₀H₂₄BFN₂O₅ 402.18 m/z, found 403.2 [M+H]⁺.

Step E: (R)-3-Amino-4-cyclopropyl-6-(3-fluoro-5-(5-(3-hydroxy-1-methyl-2-oxopyrrolidin-3-yl)isoxazol-3-yl)phenyl)picolinamide. A mixture consisting of (R)-3-(3-(3-fluoro-5-(4,4,5,5-tetramethyl-1,3,2-dioxaborolan-2-yl)phenyl)isoxazol-5-yl)-3-hydroxy-1-methylpyrrolidin-2-one (340 mg, 0.85 mmol, in 10 mL of 1,4-dioxane), 3-amino-6-chloro-4-cyclopropylpicolinamide (150 mg, 0.71 mmol, Example 88 Step A), K₃PO₄ (289 mg, 2.13 mmol) and water (1 mL) was sparged with N₂ for 5 min, treated with Pd(dtbpf)Cl₂ (46 mg, 0.071 mmol), and heated at 100° C. for 2 h under a N₂ atmosphere. After this time, the reaction vessel was removed from the oil bath and allowed to gradually cool to rt. The mixture was filtered through a pad of diatomaceous earth, and the pad washed with EtOH (15 mL). The filtrate was concentrated in vacuo. The residue was subjected to HPLC (Boston Green ODS column, 5 μm, 150×30 mm; 32-62% (v/v) CH₃CN/H₂O with 0.225% FA) to give, after lyophilization (R)-3-amino-4-cyclopropyl-6-(3-fluoro-5-(5-(3-hydroxy-1-methyl-2-oxopyrrolidin-3-yl)isoxazol-3-yl)phenyl)picolinamide (49.5 mg, 15%) as a white solid. MS (ESI): Mass calcd. for C₂₃H₂₂FN₅O₄ 451.17 m/z, found 452.1 [M+H]⁺. ¹H NMR (400 MHz, DMSO-d₆) δ 8.36 (d, J=1.6 Hz, 1H), 8.29 (s, 1H), 8.23 (d, J=10.4 Hz, 1H), 7.68 (s, 1H), 7.62 (d, J=9.2 Hz, 1H), 7.48 (s, 1H), 7.31 (s, 1H), 7.19 (br s, 2H), 6.77 (s, 1H), 3.54-3.40 (m, 2H), 2.85 (s, 3H), 2.62-2.54 (m, 1H), 2.33-2.24 (m, 1H), 1.90-1.79 (m, 1H), 1.05-0.98 (m, 2H), 0.87-0.80 (m, 2H).

### Example 244: (R)-3-Amino-4-cyclopropyl-6-(4-fluoro-3-(5-(3-hydroxy-1-methyl-2-oxopyrrolidin-3-yl)isoxazol-3-yl)phenyl)picolinamide

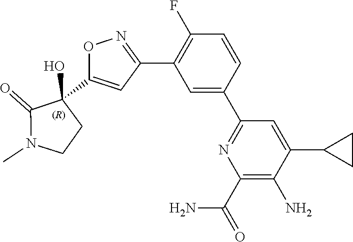

Step A: 5-Bromo-2-fluorobenzaldehyde oxime. NH₂OH·HCl (1.7 g, 25 mmol) was added to a mixture consisting of 5-bromo-2-fluorobenzaldehyde (5.0 g, 25 mmol), NaOAc (4.0 g, 49 mmol) and EtOH (60 mL). The resulting mixture was heated at 45° C. for 1 h. After this time, the mixture was poured into water (50 mL) and extracted with EtOAc (50 mL×3). The combined organic layers were washed with brine (50 mL), dried over anhydrous Na₂SO₄, filtered, and concentrated to dryness in vacuo to afford 5-bromo-2-fluorobenzaldehyde oxime (5.3 g, 98%) as a white solid. MS (ESI): Mass calcd. for C₇H₅BrFNO 216.95 m/z, found 217.7 [M+H]⁺.

Step B: 5-Bromo-2-fluoro-N-hydroxybenzimidoyl chloride. 1-Chloropyrrolidine-2,5-dione (3.4 g, 25 mmol) was added to a mixture consisting of 5-bromo-2-fluorobenzaldehyde oxime (5.0 g, 23 mmol) and DMF (50 mL). The resulting mixture was stirred at room temperature for 4 h. After this time, the mixture was diluted with H₂O (50 mL) and extracted with MTBE (50 mL×3). The combined organic extracts were washed with 4% aqueous LiCl solution (100 mL) and brine (50 mL), dried over anhydrous Na₂SO₄, filtered, and concentrated to dryness in vacuo to give 5-bromo-2-fluoro-N-hydroxybenzimidoyl chloride (6 g, 85%) as a white solid. MS (ESI): Mass calcd. for C₇H₄BrClFNO 250.91 m/z, found 251.9 [M+H]⁺.

Step C: (R)-3-(3-(5-Bromo-2-fluorophenyl)isoxazol-5-yl)-3-hydroxy-1-methylpyrrolidin-2-one. Triethylamine (0.77 mL, 5.5 mmol) was added to a mixture consisting of 5-bromo-2-fluoro-N-hydroxybenzimidoyl chloride (700 mg, 2.77 mmol) and (R)-3-ethynyl-3-hydroxy-1-methylpyrrolidin-2-one (Intermediate 1, 386 mg, 2.77 mmol) in DCM (10 mL). The resulting mixture was stirred for 16 h at room temperature. After this time, the mixture was diluted with H₂O (10 mL) and extracted with DCM (10 mL×3). The combined organic extracts were washed with brine (10 mL), dried over anhydrous Na₂SO₄, filtered, and concentrated to dryness in vacuo. The residue was subjected to silica gel chromatography (0-25% EtOAc/petroleum ether) to afford (R)-3-(3-(5-bromo-2-fluorophenyl)isoxazol-5-yl)-3-hydroxy-1-methylpyrrolidin-2-one (700 mg, 71%) as a white solid. MS (ESI): Mass calcd. for C₁₄H₁₂BrFN₂O₃ 354.00 m/z, found 355.0 [M+H]⁺. ¹H NMR (400 MHz, CHLOROFORM-d) δ 8.00-7.93 (m, 1H), 7.52-7.45 (m, 1H), 7.07-6.95 (m, 1H), 6.77 (d, J=3.5 Hz, 1H), 4.55 (br s, 1H), 3.68-3.58 (m, 1H), 3.52-3.43 (m, 1H), 2.98 (s, 3H), 2.79-2.68 (m, 1H), 2.50-2.40 (m, 1H).

Step D: (R)-3-(3-(2-Fluoro-5-(4,4,5,5-tetramethyl-1,3,2-dioxaborolan-2-yl)phenyl)isoxazol-5-yl)-3-hydroxy-1-methylpyrrolidin-2-one. [1,1′-Bis(diphenylphosphino)ferrocene]dichloropalladium(II) (84 mg, 0.12 mmol) was added to a mixture consisting of (R)-3-(3-(5-bromo-2-fluorophenyl)isoxazol-5-yl)-3-hydroxy-1-methylpyrrolidin-2-one (410 mg, 1.15 mmol), (BPin)₂ (586 mg, 2.31 mmol), KOAc (340 mg, 3.46 mmol) and 1,4-dioxane (5 mL). The resulting mixture was sparged with N₂ for 5 min, and then stirred for 3 hours at 90° C. The reaction vessel was removed from the oil bath and allowed to gradually cool to rt. The mixture was poured into water (10 mL) and extracted with EtOAc (10 mL×3). The combined organic layers were washed with brine (10 mL), dried over anhydrous Na₂SO₄, filtered, and concentrated to dryness under reduced pressure. The residue was subjected to silica gel chromatography (0-100% EtOAc/petroleum ether) to afford (R)-3-(3-(2-fluoro-5-(4,4,5,5-tetramethyl-1,3,2-dioxaborolan-2-yl)phenyl)isoxazol-5-yl)-3-hydroxy-1-methylpyrrolidin-2-one as a yellow solid (290 mg, 62%). MS (ESI): Mass calcd. for C₂₀H₂₄BFN₂O₅ 402.22 m/z, found 403.2 [M+1]⁺.

Step E: (R)-3-Amino-4-cyclopropyl-6-(4-fluoro-3-(5-(3-hydroxy-1-methyl-2-oxopyrrolidin-3-yl)isoxazol-3-yl)phenyl)picolinamide. 1,1′-Bis(di-tert-butylphosphino)ferrocene palladium dichloride (47 mg, 0.072 mmol) was added to a mixture consisting of (R)-3-(3-(2-fluoro-5-(4,4,5,5-tetramethyl-1,3,2-dioxaborolan-2-yl)phenyl)isoxazol-5-yl)-3-hydroxy-1-methylpyrrolidin-2-one (290 mg, 0.721 mmol), 3-amino-6-chloro-4-cyclopropylpicolinamide (153 mg, 0.721 mmol, Example 88 Step A) and K₃PO₄ (459 mg, 2.16 mmol) in 1,4-dioxane (4 mL) and H₂O (1 mL) under a N₂ atmosphere. The resulting mixture was sparged with N₂ for 5 min, and then stirred for 1 hour at 90° C. via microwave irradiation. The reaction vessel was removed from the microwave heating device and allowed to gradually cool to rt. The mixture was concentrated to dryness under reduced pressure. The residue was subjected to silica gel chromatography (0-100% EtOAc/petroleum ether), and then purified by HPLC (Welch Xtimate C18 column, 5 μm, 150×30 mm; 30-60% MeCN/water (0.05% NH₃H₂O+10 mM NH₄HCO₃)) to afford the title compound as a colorless solid (58 mg, 18%). MS (ESI): Mass calcd. for C₂₃H₂₂BFN₅O₄ 451.17 m/z, found 452.1 [M+1]⁺. ¹H NMR (400 MHz, DMSO-d₆) δ 8.44-8.39 (m, 1H), 8.37-8.31 (m, 1H), 8.25-8.18 (m, 1H), 7.58 (s, 1H), 7.49-7.39 (m, 2H), 7.09 (br s, 2H), 6.99-6.96 (m, 1H), 6.78 (s, 1H), 3.54-3.45 (m, 2H), 2.85 (s, 3H), 2.62-2.55 (m, 1H), 2.35-2.24 (m, 1H), 1.88-1.80 (m, 1H), 1.05-0.98 (m, 2H), 0.83-0.77 (m, 2H).

### Example 245: (R)-3-Amino-4-cyclopropyl-6-(2-fluoro-5-(5-(3-hydroxy-1-methyl-2-oxopyrrolidin-3-yl)isoxazol-3-yl)phenyl)picolinamide

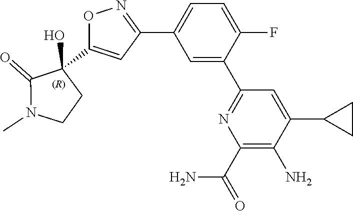

Step A: (R)-3-(3-(4-Fluoro-3-(4,4,5,5-tetramethyl-1,3,2-dioxaborolan-2-yl)phenyl)isoxazol-5-yl)-3-hydroxy-1-methylpyrrolidin-2-one. A mixture consisting of (R)-3-(3-(3-bromo-4-fluorophenyl)isoxazol-5-yl)-3-hydroxy-1-methylpyrrolidin-2-one (260 mg, 0.73 mmol, Example 240 Step C), (BPin)₂ (557 mg, 2.2 mmol), KOAc (216 mg, 2.2 mmol) and anhydrous 1,4-dioxane (5 mL) was sparged with N₂ for 5 min, treated with X-Phos-Pd-G2 (58 mg, 0.073 mmol), and stirred for 18 h at 100° C. under a N₂ atmosphere. The reaction vessel was removed from the oil bath and allowed to gradually cool to rt. The reaction mixture was used for the next step directly without work-up and purification. MS (ESI): Mass calcd. for C₂₀H₂₄BFN₂O₅ 402.17 m/z, found 403.0 [M+H]⁺.

Step B: (R)-3-Amino-4-cyclopropyl-6-(2-fluoro-5-(5-(3-hydroxy-1-methyl-2-oxopyrrolidin-3-yl)isoxazol-3-yl)phenyl)picolinamide. A mixture consisting of (R)-3-(3-(4-fluoro-3-(4,4,5,5-tetramethyl-1,3,2-dioxaborolan-2-yl)phenyl)isoxazol-5-yl)-3-hydroxy-1-methylpyrrolidin-2-one (Example 120 Step A, 234 mg, 0.58 mmol, in 5 mL of 1,4-dioxane), 3-amino-6-chloro-4-cyclopropylpicolinamide (103 mg, 0.49 mmol, Example 88 Step A), K₃PO₄ (198 mg, 1.45 mmol), and water (0.5 mL) was sparged with N₂ for 5 min, treated with Pd(dtbpf)Cl₂ (31.6 mg, 0.048 mmol), and heated at 100° C. for 4 h under N₂ atmosphere. The reaction vessel was removed from the oil bath and allowed to gradually cool to rt. The mixture was filtered through a pad of diatomaceous earth, and the pad washed with EtOAc (15 mL). The filtrate was concentrated in vacuo. The residue was subjected to HPLC (Boston Green ODS column, 5 μm, 150×30 mm; 32-62% (v/v) CH₃CN/H₂O with 0.225% FA) to give, after lyophilization, (R)-3-amino-4-cyclopropyl-6-(2-fluoro-5-(5-(3-hydroxy-1-methyl-2-oxopyrrolidin-3-yl)isoxazol-3-yl)phenyl)picolinamide (21 mg, 9.6%) as a white solid. MS (ESI): Mass calcd. for C₂₃H₂₂FN₅O₄ 451.17 m/z, found 452.2 [M+H]⁺. ¹H NMR (400 MHz, DMSO-d₆) δ 8.34 (dd, J=2.0, 7.6 Hz, 1H), 8.14 (d, J=2.4 Hz, 1H), 7.95-7.84 (m, 1H), 7.49 (br d, J=2.4 Hz, 1H), 7.46-7.37 (m, 2H), 7.27-7.05 (m, 3H), 6.72 (s, 1H), 3.51-3.43 (m, 2H), 2.84 (s, 3H), 2.59-2.54 (m, 1H), 2.31-2.22 (m, 1H), 1.90-1.81 (m, 1H), 1.06-0.98 (m, 2H), 0.70-0.63 (m, 2H). ¹H NMR (400 MHz, CD₃OD) 8.44 (dd, J=2.4, 7.6 Hz, 1H), 7.88-7.79 (m, 1H), 7.57 (d, J=2.0 Hz, 1H), 7.32 (dd, J=8.4, 11.2 Hz, 1H), 6.99 (s, 1H), 3.64-3.55 (m, 2H), 2.98 (s, 3H), 2.79-2.71 (m, 1H), 2.46-2.36 (m, 1H), 1.87-1.77 (m, 1H), 1.14-1.07 (m, 2H), 0.74-0.67 (m, 2H).

Certain compounds described herein were tested in biological assays. The results of the assays are presented in Table 3 which is entitled Results of Biological Assays. The results are presented as an average of values obtained.

### Biological Assay 1: Inhibition of Auto-Phosphorylation of Recombinant Human NF-Kappab-Inducing Kinase (NIK/MAP3K14) Activity (AlphaScreen®)

NIK/MAP3K14 auto-phosphorylation activity was measured using the AlphaScreen® ((αscreen) format (Perkin ElmeR). All compounds tested were dissolved in dimethyl sulfoxide (DMSO) and further dilutions were made in assay buffer. Final DMSO concentration was 0.7% (v/v) in assays. Assay buffer was 50 mM Tris pH 7.5 containing 1 mM EGTA (ethylene glycol tetraacetic acid), 1 mM DTT (dithiothreitol), 0.1 mM Na₃VO₄, 5 mM MgCl₂, 0.01% Tween® 20. Assays were carried out in 384 well Proxiplates (Perkin ElmeR). Incubations consisted of compound, 5 microM Adenosine-5′-triphosphate (ATP), and 1 nM NIK/MAP3K14. Incubations were initiated by addition of GST-tagged NIK/MAP3K14 enzyme, carried out for 2 h at 25° C. and terminated by addition of stop buffer containing anti-phospho-IKK Ser176/180 antibody. Protein A Acceptor and Glutathione-donor beads were added before reading using an EnVision® Multilabel Plate Reader (Perkin ElmeR). Signal obtained in the wells was normalized using high (full enzyme activity, 0.7% DMSO) and low controls (no enzyme activity, 0.7% DMSO, no ATP). IC₅₀'s were determined by fitting a sigmoidal curve to % inhibition of control versus Log₁₀ compound concentration.

Biological Assay 2: Effect of Compounds on p-IKKα Levels in L363 (NIK Translocated Multiple Myeloma) Cells

All compounds tested were dissolved and serially diluted in DMSO, 1:3 dilution for 11 points in an Echo compatible plate. 100% DMSO is added to columns 12 and 24 of the plate to serve as high and low signal controls. This compound plate was used to spot 20 nl of compound or DMSO into a Greiner 384 well TC plate (781080). Final DMSO concentration was 0.3% (v/v) in cell assays. Human L363 cells (ATCC) were cultured in RPMI 1640 medium supplemented with GlutaMax, non-essential amino acids, sodium pyruvate and 10% fetal bovine serum. Cells were routinely maintained at densities of 0.2×10⁶ cells per ml −2×10⁶ cells per ml at 37° C. in a humidified 5% CO₂ atmosphere incubator. Cells were passaged twice a week splitting back to obtain the low density. The day before the assay, cells were washed twice in HBSS, resuspended in DMEM+0.5% IgG and protease free BSA (Jackson Immuno Research Laboratories), +/−250 ng/ml recombinant human B-cell activating factor (BAFF/BLyS/TNFSF13B) and incubated overnight at 37° C. in a humidified 5% CO₂ atmosphere (bulk stimulation with or without BAFF). The next day, cell concentration was adjusted to 1×10⁷ cells/ml in DMEM+/−250 ng/ml BAFF+/−10 uM MG132 and plated at 10 ul/well into compound or DMSO spotted 384 well TC plates. Seeded cells were incubated at 37° C. in a humidified 5% CO₂ atmosphere for 6 h. After 6 h, plates were removed from the incubator and cell lysis was achieved by the addition of 2.5 μl 5× lysis buffer containing protease and phosphatase inhibitors, followed by shaking on a plate shaker at room temperature for 15 min. At the end of this incubation, lysed cells were sequentially treated and incubated with acceptor and donor bead mixes according to the manufacturer's protocol for a 1 plate/2-incubation suspension cell assay (AlphaLISA SureFire Ultra p-IKKα (Ser 176/180) Assay Kit (Perkin ElmeR). Plates were read using an EnVision® Multilabel Plate Reader (Perkin ElmeR). Within an experiment, a concentration response curve for each compound was run in duplicate. Signal obtained in the test wells was normalized using high signal (BAFF stimulated cells, DMSO, MG132) and low signal (unstimulated cells, DMSO) controls. To determine the IC₅₀, a sigmoidal curve was fitted to the plot of % inhibition versus Log₁₀ compound concentration.

Table 3 below provides IC₅₀ data for certain compounds described herein on NIK inhibition. In cases where the compound was tested more than once, the IC₅₀ value was calculated as a simple average of the measured values. The average was then used to categorize the compound within one of the following ranges.
A: IC₅₀<100 nM;
B: 100 nM≤IC₅₀≤500 nM;
C: IC₅₀>501 nM

TABLE 3
Results of Biological Assays

| Example | Assay 1 IC₅₀ | Assay 2 IC₅₀ |
|---|---|---|
| 1 | A | B |
| 2 | A | B |
| 3 | A | C |
| 4 | A | C |
| 5 | B | C |
| 6 | B | C |
| 7 | A | A |
| 8 | A | B |
| 9 | B | C |
| 10 | A | C |
| 11 | A | C |
| 12 | A | C |
| 13 | A | C |
| 14 | A | C |
| 15 | A | A |
| 16 | A | B |
| 17 | A | C |
| 18 | A | C |
| 19 | A | C |
| 20 | B | C |
| 21 | A | C |
| 22 | A | A |
| 23 | A | A |
| 24 | B | C |
| 25 | B | C |
| 26 | A | A |
| 27 | C | C |
| 28 | B | C |
| 29 | C | C |
| 30 | C | C |
| 31 | B | C |
| 32 | A | C |
| 33 | C | C |
| 34 | C | C |
| 35 | A | C |
| 36 | B | C |
| 37 | C | C |
| 38 | A | C |
| 39 | C | C |
| 40 | C | C |
| 41 | C | C |
| 42 | C | C |
| 43 | B | C |
| 44 | C | C |
| 45 | A | A |
| 46 | A | A |
| 47 | C | C |
| 48 | A | C |
| 49 | A | C |
| 50 | A | A |
| 51 | B | C |
| 52 | B | C |
| 53 | B | C |
| 54 | B | C |
| 55 | C | C |
| 56 | C | C |
| 57 | C | C |
| 58 | B | C |
| 59 | C | C |
| 60 | C | C |
| 61 | B | C |
| 62 | C | C |
| 63 | C | C |
| 64 | C | C |
| 65 | C | C |
| 66 | C | C |
| 67 | B | C |
| 68 | C | C |
| 69 | C | C |
| 70 | C | C |
| 71 | C | C |
| 72 | C | C |
| 73 | C | C |
| 74 | C | C |
| 75 | B | C |
| 76 | B | C |
| 77 | C | C |
| 78 | B | C |
| 79 | C | C |
| 80 | C | C |
| 81 | C | C |
| 82 | C | C |
| 83 | C | C |
| 84 | C | C |
| 85 | A | C |
| 86 | A | C |
| 87 | C | C |
| 88 | A | A |
| 89 | A | B |
| 90 | B | C |
| 91 | A | B |
| 92 | B | C |
| 93 | A | A |
| 94 | C | C |
| 95 | C | C |
| 96 | A | C |
| 97 | A | B |
| 98 | A | C |
| 99 | A | A |
| 100 | A | C |
| 101 | A | B |
| 102 | A | C |
| 103 | A | A |
| 104 | A | A |
| 105 | A | A |
| 106 | A | B |
| 107 | A | A |
| 108 | A | A |
| 109 | A | B |
| 110 | A | A |
| 111 | A | A |
| 112 | A | B |
| 113 | A | C |
| 114 | A | C |
| 115 | C | C |
| 116 | A | C |
| 117 | A | B |
| 118 | A | B |
| 119 | A | B |
| 120 | A | C |
| 121 | A | B |
| 122 | B | C |
| 123 | A | B |
| 124 | A | C |
| 125 | A | C |
| 126 | A | C |
| 127 | A | A |
| 128 | A | C |
| 129 | A | A |
| 130 | A | A |
| 131 | C | C |
| 132 | A | A |
| 133 | A | C |
| 134 | A | A |
| 135 | A | A |
| 136 | B | C |
| 137 | A | A |
| 138 | C | C |
| 139 | A | A |
| 140 | A | A |
| 141 | A | A |
| 142 | A | B |
| 143 | A | A |
| 144 | A | A |
| 145 | A | A |
| 146 | A | C |
| 147 | A | C |
| 148 | A | C |
| 149 | C | C |
| 150 | C | C |
| 151 | C | C |
| 152 | A | C |
| 153 | A | C |
| 154 | A | C |
| 155 | A | B |
| 156 | B | C |
| 157 | C | C |
| 158 | A | C |
| 159 | A | B |
| 160 | A | C |
| 161 | A | C |
| 162 | A | C |
| 163 | A | C |
| 164 | A | C |
| 165 | A | C |
| 166 | A | B |
| 167 | A | B |
| 168 | A | C |
| 169 | A | A |
| 170 | A | C |
| 171 | A | C |
| 172 | C | C |
| 173 | A | C |
| 174 | B | C |
| 175 | A | C |
| 176 | C | C |
| 177 | A | A |
| 178 | A | C |
| 179 | A | A |
| 180 | A | B |
| 181 | A | B |
| 182 | A | C |
| 183 | A | C |
| 184 | A | B |
| 185 | A | C |
| 186 | A | B |
| 187 | A | B |
| 188 | A | C |
| 189 | A | B |
| 190 | A | C |
| 191 | A | B |
| 192 | A | C |
| 193 | A | C |
| 194 | C | C |
| 195 | A | C |
| 196 | A | C |
| 197 | C | C |
| 198 | B | C |
| 199 | A | C |
| 200 | A | C |
| 201 | A | C |
| 202 | A | C |
| 203 | A | C |
| 204 | A | C |
| 205 | A | C |
| 206 | A | A |
| 207 | A | C |
| 208 | A | B |
| 209 | A | A |
| 210 | A | A |
| 211 | A | A |
| 212 | C | C |
| 213 | A | A |
| 214 | C | C |
| 215 | A | C |
| 216 | A | C |
| 217 | A | B |
| 218 | A | B |
| 219 | A | C |
| 220 | A | C |
| 221 | A | C |
| 222 | A | B |
| 223 | A | C |
| 224 | B | C |
| 225 | B | C |
| 226 | C | C |
| 227 | B | C |
| 228 | B | C |
| 229 | C | C |
| 230 | B | C |
| 231 | A | C |
| 232 | C | C |
| 233 | C | C |
| 234 | C | C |
| 235 | A | A |
| 236 | A | A |
| 237 | A | C |
| 238 | A | B |
| 239 | A | A |
| 240 | A | C |
| 241 | A | A |
| 242 | A | A |
| 243 | A | A |
| 244 | A | C |
| 245 | A | A |

1. A compound of Formula I: 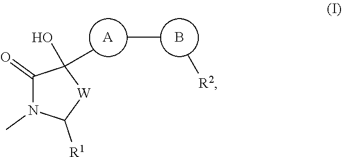
or a pharmaceutically acceptable salt thereof, wherein:
A is a 5-membered heteroaryl that is optionally substituted with one to three groups selected from —C₍₁₋₄₎alkyl and —C₍₁₋₄₎haloalkyl;
B is furanyl, piperidinyl, or a group having the following structure: 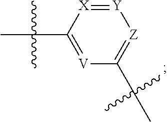
W is CH₂, CHF, or CF₂;
V is N, C—H, or C—R^{V};
X is N, C—H, or C—R^{X};
Y is N, C—H, or C—R^{Y};
Z is N, C—H, or C—R^{Z};
R^{V}, R^{X}, R^{Y}, and R^{Z} are each, independently, halo, —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein the —N(H)(5- to 10-membered heteroaryl), the —C₍₆₋₁₀₎aryl, the 5- to 12-membered bi- or tricyclic ring system containing one or more heteroatoms, and the 5- to 10-membered heteroaryl are each optionally substituted with one to five R³ groups;
each R³ is independently halo, —OH, —N(R^{N1})(R^{N2}), —CN, —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎cycloalkyl, —C₍₁₋₃₎alkyl-N(R^{N1})(R^{N3}), —C(O)OH, —C(O)N(R^{N1})(R^{N4}), —C₍₁₋₃₎alkyl(3- to 8-membered heterocyclyl), —C₍₁₋₃₎ alkyl(phenyl), 3- to 8-membered heterocyclyl, or 5- to 6-membered heteroaryl, wherein the —C₍₁₋₆₎alkyl, —C₍₁₋₆₎haloalkyl, —C₍₃₋₁₀₎cycloalkyl, —OC₍₁₋₆₎alkyl, —OC₍₁₋₆₎haloalkyl, —OC₍₃₋₁₀₎ cycloalkyl, —C₍₁₋₃₎alkyl(3- to 8-membered heterocyclyl), —C₍₁₋₃₎alkyl(phenyl), 3- to 8-membered heterocyclyl, and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎haloalkyl, and —C₍₁₋₄₎alkylOC₍₁₋₄₎alkyl;
R^{N1} is hydrogen or C₍₁₋₄₎alkyl;
R^{N2}, R^{N3}, and R^{N4} are each independently for each occurrence hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R³ groups.

2-4. (canceled)

5. The compound of claim 1, or a pharmaceutically acceptable salt thereof, wherein:
A is a 5-membered heteroaryl that is optionally substituted with one —C₍₁₋₄₎alkyl group;
B is: 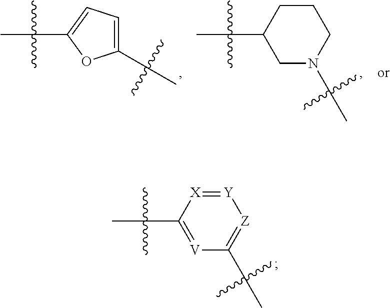
W is CH₂ or CF₂;
V is N, C—H, or C—F;
X is N, C—H, or C—R^{X};
Y is N, C—H, or C—R^{Y};
Z is N, C—H, or C—R^{Z};
R^{X}, R^{Y}, and R^{Z} are each, independently, fluorine, —CH₃, or —OCH₃;
R¹ is hydrogen or —CF₃;
R² is —N(H)(5- to 10-membered heteroaryl), —C₍₆₋₁₀₎aryl, a 5- to 12-membered bi- or tricyclic ring system containing one or more heteroatoms, or 5- to 10-membered heteroaryl, wherein: the —N(H)(5- to 10-membered heteroaryl) is optionally substituted with one —NH₂ group, the —C₍₆₋₁₀₎aryl is optionally substituted with one —C(O)NH₂ group, the 5- to 12-membered bi- or tricyclic ring system containing one or more heteroatoms is optionally substituted with one to five R^{3c} groups, and the 5- to 10-membered heteroaryl is optionally substituted with one to five R^{3d} groups;
each R^{3c} is independently for each occurrence —NH₂, —C₍₁₋₆₎alkyl, —C₍₁₋₆₎haloalkyl, —OC₍₁₋₆₎alkyl, —C(O)OH, —C(O)NH₂, or —C₍₁₋₃₎alkyl(phenyl);
each R^{3d} is independently for each occurrence halo, —N(R^{N1})(R^{N2}), —C₍₁₋₆₎alkyl, —C₍₁₋₆₎ haloalkyl, —C₍₃₋₁₀₎cycloalkyl, —OC₍₁₋₆₎alkyl, —OC₍₁₋₆₎haloalkyl, —C₍₁₋₃₎alkyl-N(R^{N1})(R^{N3}), —C(O)N(R^{N1})(R^{N4}), —C₍₁₋₃₎alkyl(3- to 8-membered heterocyclyl), 3- to 8-membered heterocyclyl, or 5- to 6-membered heteroaryl, wherein the 3- to 8-membered heterocyclyl and 5- to 6-membered heteroaryl are optionally further substituted with one to five groups selected from halo, —C₍₁₋₄₎alkyl, —C₍₁₋₄₎haloalkyl, —C₍₃₋₆₎cycloalkyl, —OC₍₁₋₄₎alkyl, —OC₍₁₋₄₎ haloalkyl, and —C₍₁₋₄₎alkylOC₍₁₋₄₎alkyl;
R^{N1} is hydrogen;
R^{N2} is hydrogen, —C₍₁₋₄₎alkyl, —C₍₃₋₆₎cycloalkyl, —C(O)C₍₁₋₄₎alkyl, —C₍₁₋₄₎alkyl(4- to 6-membered heterocyclyl), 4- to 6-membered heterocyclyl, 5- to 6-membered heteroaryl, wherein the 4- to 6-membered heterocyclyl and 5- to 6-membered heteroaryl are optionally further substituted with one to three groups selected from —CN, —C₍₁₋₄₎alkyl, —C₍₁₋₄₎haloalkyl, —OC₍₁₋₄₎alkyl, —OC₍₁₋₄₎haloalkyl, and —C(O)C₍₁₋₄₎alkyl;
R^{N3} is hydrogen, —C₍₃₋₆₎cycloalkyl, or 5- to 6-membered heteroaryl, wherein the 5- to 6-membered heteroaryl is optionally further substituted with one to three —C₍₁₋₄₎alkyl groups;
R^{N4} is hydrogen, —C₍₁₋₄₎alkyl, or 4- to 6-membered heterocyclyl;
provided that at least two of V, X, Y, and Z are C—H; and
provided that if R² is pyrimidinyl, then the pyrimidinyl is substituted with one group selected from —OC₍₁₋₆₎alkyl and —C(O)N(R^{N1})(R^{N4}) and is optionally further substituted with one to two R^{3d} groups.

6-9. (canceled)

10. The compound of claim 1, or a pharmaceutically acceptable salt thereof, wherein A is

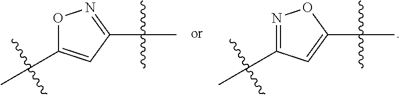 [SMILES: *c1cc(*)on1 ; *c1cc(*)on1]

11. The compound of claim 1, or a pharmaceutically acceptable salt thereof, wherein W is CH₂.

12. (canceled)

13. The compound of claim 1, or a pharmaceutically acceptable salt thereof, wherein B is

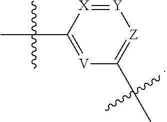

14-16. (canceled)

17. The compound of claim 1, or a pharmaceutically acceptable salt thereof, wherein B is

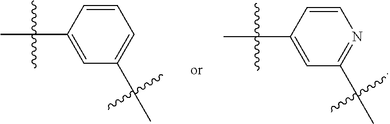 [SMILES: *c1cccc(*)c1 ; *c1ccnc(*)c1]

18. (canceled)

19. (canceled)

20. The compound of claim 1, or a pharmaceutically acceptable salt thereof, which is a compound of any one of Formulas Ie-1 to Ie-6:

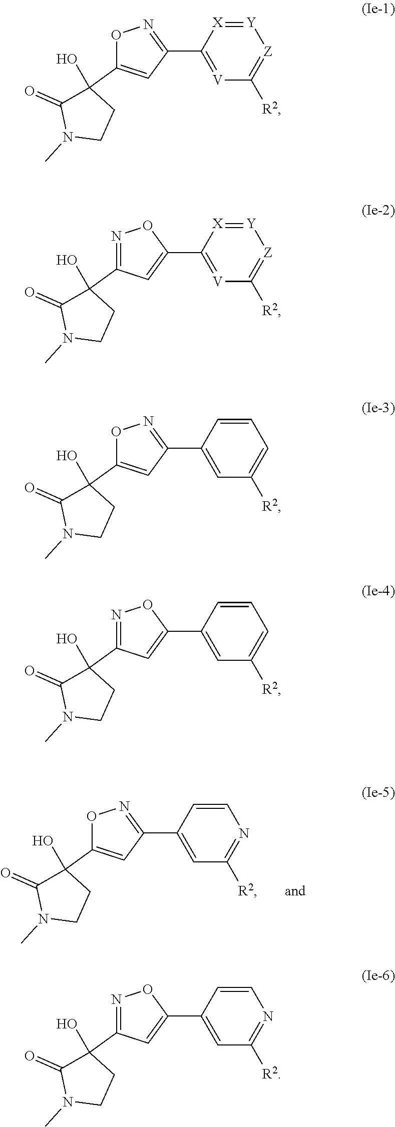

21. The compound of claim 1, or a pharmaceutically acceptable salt thereof, wherein R² is —N(H)(5- to 10-membered heteroaryl), which is optionally substituted with one to five R^{3a} groups.

22. (canceled)

23. The compound of claim 1, or a pharmaceutically acceptable salt thereof, wherein R^{3a} is —N(R^{N1})(R^{N2}).

24-28. (canceled)

29. The compound of claim 1, or a pharmaceutically acceptable salt thereof, wherein R² is

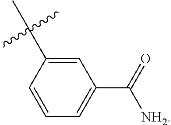 [SMILES: *c1cccc(C(N)=O)c1]

30-34. (canceled)

35. The compound of claim 1, or a pharmaceutically acceptable salt thereof, wherein R² is:

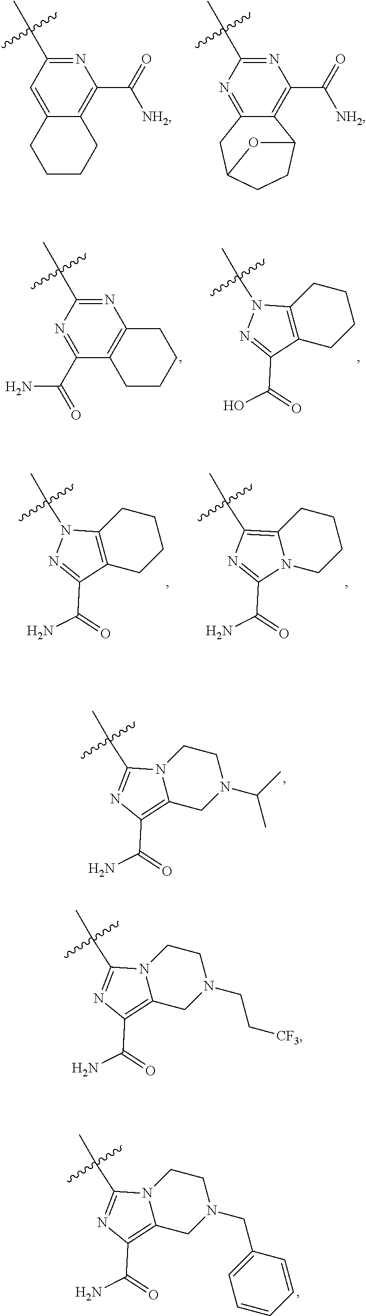

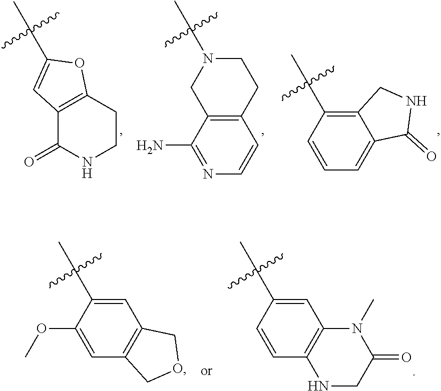

36-38. (canceled)

39. The compound of claim 1, or a pharmaceutically acceptable salt thereof, wherein R² is pyrazolyl, imidazolyl, furanyl, thiazolyl, pyridinyl, pyrimidinyl, pyrazinyl, indolyl, indazolyl, benzimidazolyl, pyrrolopyridinyl, pyrrolopyrimidinyl, pyrrolopyrazinyl, pyrrolopyridazinyl, pyrrolotriazinyl, pyrazolopyridinyl, pyrazolopyrimidinyl, imidazopyridinyl, imidazopyrazinyl, imidazopyridazinyl, triazolopyridinyl, quinazolinyl, isoquinolinonyl, quinazolinonyl, pyridopyrimidinyl, pyridopyrimidinonyl, pyrimidopyrimidinyl, benzoxazolyl, thiazolopyrimidinyl, furopyridinyl, or thienopyrimidinyl, each of which is optionally substituted with one to three R^{3d} groups.

40. The compound of claim 1, or a pharmaceutically acceptable salt thereof, wherein R² is thiazolyl, pyridinyl, pyrimidinyl, indazolyl, pyrazolopyridinyl, or imidazopyridinyl, each of which is optionally substituted with one to three R^{3d} groups.

41-46. (canceled)

47. The compound of claim 1, or a pharmaceutically acceptable salt thereof, wherein R² is

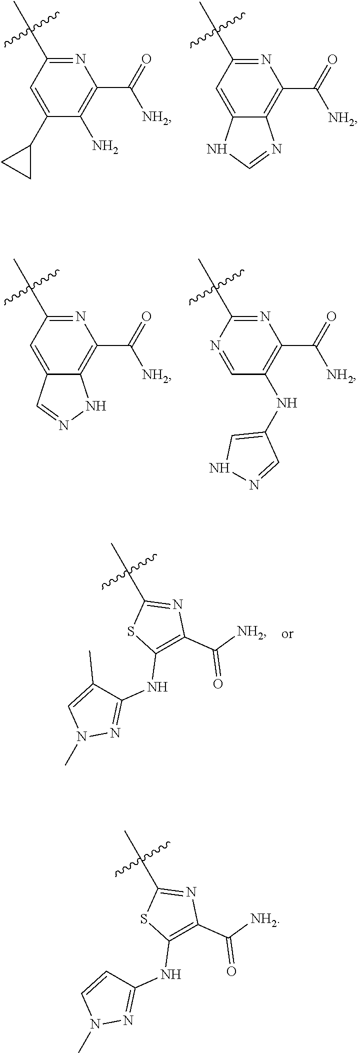 [SMILES: *c1cc(C2CC2)c(N)c(C(N)=O)n1 ; *c1cc2[nH]cnc2c(C(N)=O)n1 ; *c1cc2cn[nH]c2c(C(N)=O)n1 ; *c1ncc(Nc2cn[nH]c2)c(C(N)=O)n1 ; *c1nc(C(N)=O)c(Nc2nn(C)cc2C)s1 ; *c1nc(C(N)=O)c(Nc2ccn(C)n2)s1]

48-50. (canceled)

51. The compound of claim 1, or a pharmaceutically acceptable salt thereof, which is a compound of Formula If: 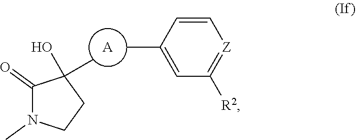
wherein:
A is a isoxazolyl;
Z is N or C—H;
R² is 5- to 10-membered heteroaryl, which is optionally substituted with one to three R^{3d} groups;
each R^{3d} is independently for each occurrence —N(R^{N1})(R^{N2}), —C₍₁₋₆₎alkyl, —C₍₁₋₆₎ haloalkyl, —C₍₃₋₁₀₎cycloalkyl, —C(O)N(R^{N1})(R^{N4}), or 5- to 6-membered heteroaryl, wherein the 5- to 6-membered heteroaryl is optionally further substituted with one to three —C₍₁₋₄₎alkyl groups;
R^{N1} is hydrogen;
R^{N2} is hydrogen, 4- to 6-membered heterocyclyl, or 5- to 6-membered heteroaryl, wherein the 5- to 6-membered heteroaryl is optionally further substituted with one to three —C₍₁₋₄₎alkyl groups; and
R^{N4} is hydrogen;
provided that if R² is pyrimidinyl, then the pyrimidinyl is substituted with one —C(O)N(R^{N1})(R^{N4}) group and is optionally further substituted with one to two R^{3d} groups.

52. (canceled)

53. The compound of claim 1, or a pharmaceutically acceptable salt thereof, having a structure selected from the group consisting of:

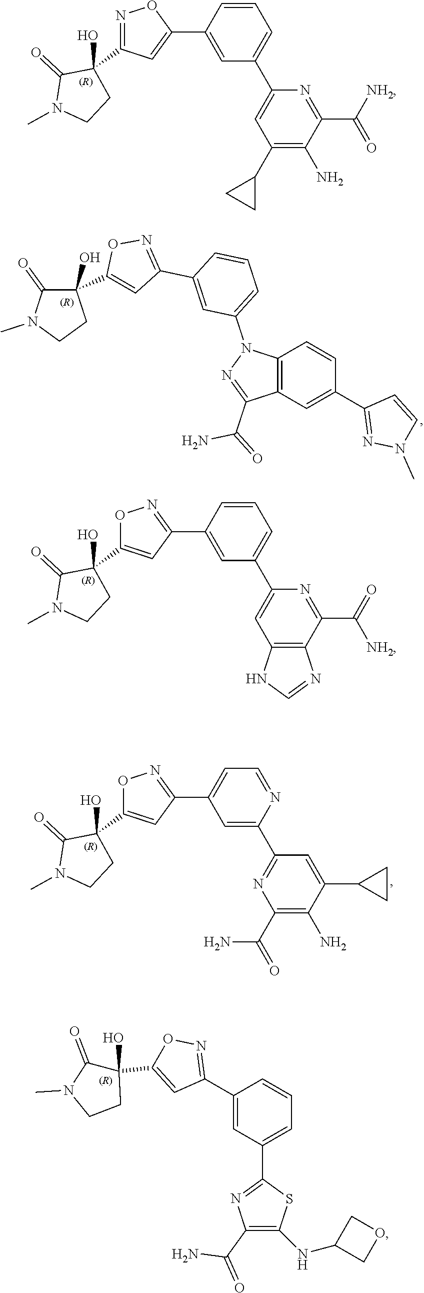

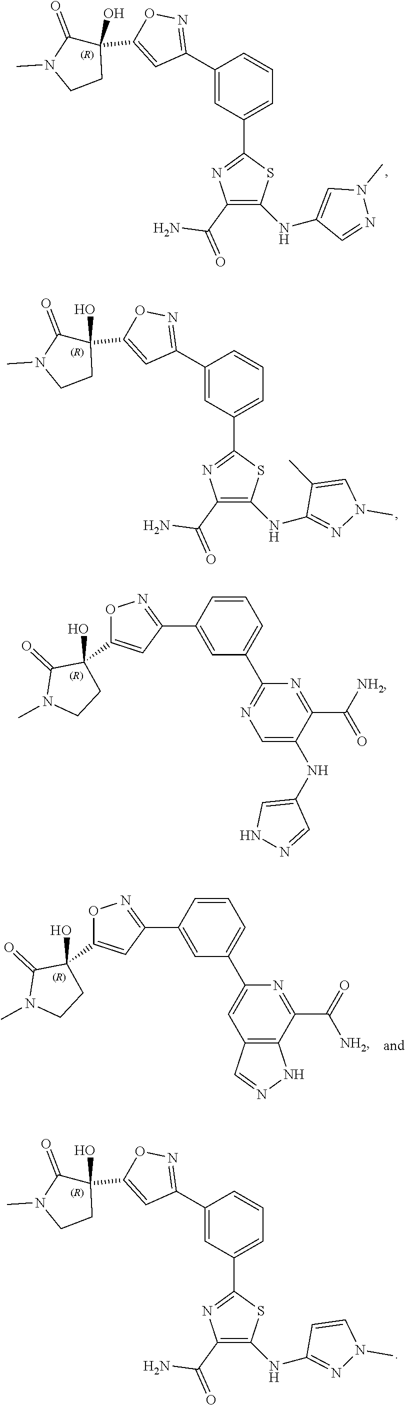

54-63. (canceled)

64. A pharmaceutical composition comprising a compound of claim 1, or a pharmaceutically acceptable salt thereof, and a pharmaceutically acceptable carrier.

65. A method of treating a disease, disorder, or medical condition mediated by NIK activity, comprising administering to a subject in need of such treatment an effective amount of (i) a compound of claim 1 or a pharmaceutically acceptable carrier thereof.

66. The method of claim 65, wherein the disease, disorder or medical condition mediated by NIK activity is selected from the group consisting of inflammatory disorders and autoimmune disorders.

67. (canceled)

68. (canceled)

69. The method of claim 1, wherein the disease, disorder or medical condition mediated by NIK activity is selected from the group consisting of systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.
